(19) United States
(12) Patent Application Publication
(10) Pub. No.: US 2024/0360455 A1
(43) Pub. Date: Oct. 31, 2024

(54) EXTRA-HEPATIC DELIVERY IRNA COMPOSITIONS AND METHODS OF USE THEREOF

(71) Applicant: Alnylam Pharmaceuticals, Inc., Cambridge, MA (US)

(72) Inventors: Jayaprakash K. Nair, Cambridge, MA (US); Justin Pierson, Wakefield, MA (US); Elena Castellanos-Rizaldos, Melrose, MA (US); Yesseinia Anglero-Rodriguez, Cambridge, MA (US); Karyn Schmidt, Melrose, MA (US); Lucas D. BonDurant, Broolline, MA (US); Kevin Dooley, Melrose, MA (US); Ivan Zlatev, Cambridge, MA (US); Vasant R. Jadhav, Sharon, MA (US); Martin A. Maier, Belmont, MA (US); Alexander V. Kel'in, Swampscott, MA (US); Masaaki Nakata, Cambridge, MA (US)

(21) Appl. No.: 18/631,104

(22) Filed: Apr. 10, 2024

Related U.S. Application Data
(63) Continuation of application No. PCT/US2022/046668, filed on Oct. 14, 2022.
(60) Provisional application No. 63/255,984, filed on Oct. 15, 2021.

(51) Int. Cl. C12N 15/113; A61K 45/06; A61P 9/10

(52) U.S. Cl. CPC C12N 15/1137; A61K 45/06; A61P 9/10; C12N 2310/11; C12N 2310/351; C12N 2320/32; C12Y 207/11017

(57) ABSTRACT

The present invention provides double stranded ribonucleic acid (dsRNA) agents for inhibiting expression of a target gene, comprising an antisense strand which is complementary to the target gene; a sense strand which is complementary to the antisense strand and forms a double stranded region with the antisense strand; and one or more C₂₂ hydrocarbon chains conjugated to one or more internal positions on at least one strand, compositions comprising such dsRNA agents, and methods of use thereof for treating a subject having a skeletal muscle disorder, a cardiac muscle disorder, or an adipose tissue disorder.

### RELATED APPLICATIONS

This application is a 35 § U.S.C. 111(a) continuation application which claims the benefit of priority to PCT/US2022/046668, filed on Oct. 14, 2022, which, in turn, claims the benefit of priority to U.S. Provisional Application No. 63/255,984, filed on Oct. 15, 2021. The entire contents of each of the foregoing applications are incorporated herein by reference.

### SEQUENCE LISTING

The instant application contains a Sequence Listing which has been submitted electronically in XML file format and is hereby incorporated by reference in its entirety. Said XML copy, created on Mar. 29, 2024, is named 121301_15302_SL.xml and is 37,892,374 bytes in size.

### BACKGROUND OF THE INVENTION

Efficient delivery of an RNAi agent to cells in vivo requires specific targeting and substantial protection from the extracellular environment, particularly serum proteins. RNAi-based therapeutics show promising clinical data for treatment of liver-associated disorders. However, RNAi delivery into extra-hepatic tissues remains an obstacle, limiting the use of RNAi-based therapies.

One of the limiting factors is the ability to deliver intact RNAi efficiently to extra-hepatic tissues, such as muscle tissues, e.g., skeletal muscle tissues and cardiac muscle tissues, and adipose tissue.

For example, when administered systemically, RNAi agents naturally accumulate in the liver limiting distribution to extra-hepatic tissues.

Similarly, particular difficulties have been associated when RNAi agents are administered locally, in that although the RNAi agents can achieve significant target gene reduction, there is limited distribution in muscle or adipose tissue and target gene reduction is only observed in a small portion of the tissue, minimizing the potential therapeutic use.

Previous work has used delivery reagents such as liposomes, cationic lipids, and nanoparticles forming complexes to aid the intracellular internalization of RNAi agents into extra-hepatic cells. However, only limited success in delivering RNAi agents to extra-hepatic tissues, like muscle tissue, after systemic administration has been reported. For example although cholesterol-conjugated RNAi agents are delivered to muscles after intravenous injection, a high dose (50 mg/kg) is required to achieve sustainable gene silencing. In addition, cholesterol conjugates are highly toxic at high concentrations, limiting their potential for clinical applications. With respect to adipose tissues, while viral carriers have shown promise for RNAi agent delivery to adipocytes, the delivery process is labor intensive and the high immunogenicity has limited the widespread application. (See, e.g., Biscans, et al. (2018) Nucl Acids Res 47(3):1082).

Thus, systemic delivery of oligonucleotides to extra-hepatic tissues, like muscle tissue and adipose tissue, remains a challenge and, accordingly, there is a continuing need for new and improved compositions and methods for delivering RNAi agents in vivo, without the use of tissue delivery reagents, to achieve and enhance the rapeutic potential of RNAi agents.

### SUMMARY OF THE INVENTION

The present invention is based, at least in part, on the surprising discovery that conjugating a C₂₂ lipophilic moiety to one or more internal positions on at least one strand of a dsRNA agent, e.g., position 6 on the sense strand, counting from the 5′-end, provides surprisingly efficient in vivo delivery to muscle and/or adipose tissue resulting in efficient entry and internalization of the dsRNA agent into muscle tissue, e.g., cardiac and skeletal tissue, and/or adipose tissue, and sparingly good inhibition of target gene expression in muscle tissue and/or adipose tissue, e.g., cardiac and skeletal tissue, and/or adipose tissue.

Accordingly, in one aspect, the present invention provides a dsRNA agent comprising an antisense strand which is complementary to a target gene; a sense strand which is complementary to the antisense strand and forms a double stranded region with the antisense strand; and one or more C₂₂ hydrocarbon chain conjugated to one or more internal positions on at least one strand, wherein the dsRNA agent is suitable for delivery to a muscle tissue, e.g., skeletal muscle tissue or cardiac muscle tissue, or an adipose tissue. In some embodiments, the one or more C₂₂ hydrocarbon chains conjugated to one or more internal positions on at least one strand are conjugated to the dsRNA agent via a linker or carrier.

In some embodiments, the lipophilicity of the one or more C₂₂ hydrocarbon chain, measured by octanol-water partition coefficient, log K_{ow}, exceeds 0. The lipophilic moiety may possess a log K_{ow} exceeding 1, exceeding 1.5, exceeding 2, exceeding 3, exceeding 4, exceeding 5, or exceeding 10.

In some embodiments, the hydrophobicity of the dsRNA agent, measured by the unbound fraction in the plasma protein binding assay of the dsRNA agent, exceeds 0.2. In one embodiment, the plasma protein binding assay determined is an electrophoretic mobility shift assay (EMSA) using human serum albumin protein. The hydrophobicity of the dsRNA agent, measured by fraction of unbound dsRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of dsRNA/

The C₂₂ hydrocarbon chain may be saturated or unsaturated.

The C₂₂ hydrocarbon chain may be linear or branched

In some embodiments, the internal positions include all positions except the three terminal positions from each end of the at least one strand.

In some embodiments, the internal positions exclude a cleavage site region of the sense strand.

In some embodiments, the internal positions exclude positions 9-12 or positions 11-13, counting from the 5′-end of the sense strand.

In some embodiments, the internal positions exclude a cleavage site region of the antisense strand.

In some embodiments, the internal positions exclude positions 12-14, counting from the 5′-end of the antisense strand.

In some embodiments, the one or more C₂₂ hydrocarbon chains are conjugated to one or more of the following internal positions: positions 4-8 and 13-18 on the sense strand, and positions 6-10 and 15-18 on the antisense strand, counting from the 5′ end of each strand.

In some embodiments, the one or more C₂₂ hydrocarbon chains are conjugated to one or more of the following internal positions: positions 5, 6, 7, 15, and 17 on the sense strand, and positions 15 and 17 on the antisense strand, counting from the 5′-end of each strand.

In some embodiments, the one or more C₂₂ hydrocarbon chains are conjugated to position 6 on the sense strand, counting from the 5′-end of the sense strand.

In some embodiments, the one or more C₂₂ hydrocarbon chains is an aliphatic, alicyclic, or polyalicyclic compound, e.g., the one or more C₂₂ hydrocarbon chains contains a functional group selected from the group consisting of hydroxyl, amine, carboxylic acid, sulfonate, phosphate, thiol, azide, and alkyne.

In some embodiments, the one or more C₂₂ hydrocarbon chains is a C22 acid, e.g. the C22 acid is selected from the group consisting of docosanoic acid, 6-octyltetradecanoic acid, 10-hexylhexadecanoic acid, all-cis-7,10,13,16,19-docosapentaenoic acid, all-cis-4,7,10,13,16,19-docosahexaenoic acid, all-cis-13,16-docosadienoic acid, all-cis-7,10,13,16-docosatetraenoic acid, all-cis-4,7,10,13,16-docosapentaenoic acid, and cis-13-docosenoic acid.

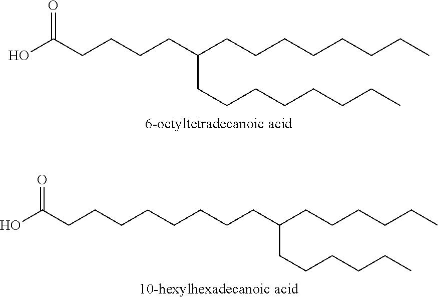 [SMILES: CCCCCCCCC(CCCCCCCC)CCCCC(=O)O ; CCCCCCC(CCCCCC)CCCCCCCCC(=O)O]

In some embodiments, the one or more C₂₂ hydrocarbon chains is a C22 alcohol, e.g., the C22 alcohol is selected from the group consisting of 1-docosanol, 6-octyltetradecan-1-ol, 10-hexylhexadecan-1-ol, cis-13-docosen-1-ol, docosan-9-ol, docosan-2-ol, docosan-10-ol, docosan-11-ol, and cis-4,7,10,13,16,19-docosahexanol.

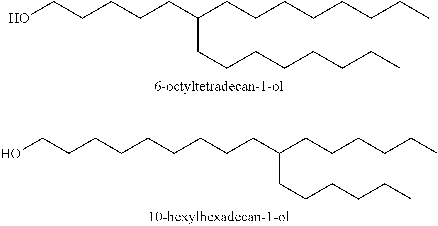 [SMILES: CCCCCCCCC(CCCCCO)CCCCCCCC ; CCCCCCC(CCCCCC)CCCCCCCCCO]

In some embodiments, the one or more C₂₂ hydrocarbon chains is a C22 amide, e.g., the C22 amide is selected from the group consisting of (E)-Docos-4-enamide, (E)-Docos-5-enamide, (Z)-Docos-9-enamide, (E)-Docos-11-enamide, 12-Docosenamide, (Z)-Docos-13-enamide, (Z)—N-Hydroxy-13-docoseneamide, (E)-Docos-14-enamide, 6-cis-Docosenamide, 14-Docosenamide Docos-11-enamide, (4E, 13E)-Docosa-4,13-dienamide, and (5E, 13E)-Docosa-5,13-dienamide.

The one or more C₂₂ hydrocarbon chains may be conjugated to the dsRNA agent via a direct attachment to the ribosugar of the dsRNA agent. Alternatively, the one or more C₂₂ hydrocarbon chains may be conjugated to the dsRNA agent via a linker or a carrier. In some embodiments, the one or more C₂₂ hydrocarbon chains may be conjugated to the dsRNA agent via internucleotide phosphate linkage.

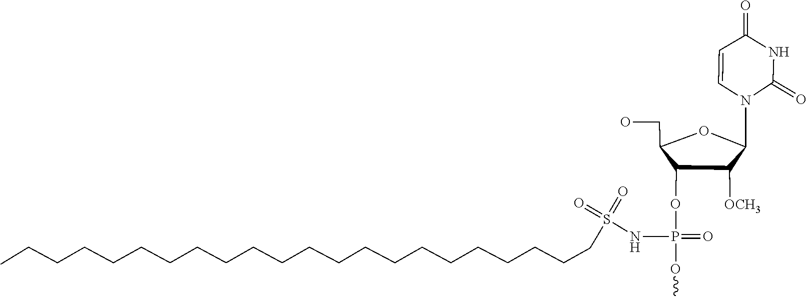

In certain embodiments, the one or more C₂₂ hydrocarbon chains is conjugated to the dsRNA agent via one or more linkers (tethers), e.g., a carrier that replaces one or more nucleotide(s) in the internal position(s).

In some embodiments, the one or more C₂₂ hydrocarbon chains is conjugated to the dsRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, at least one of the linkers (tethers) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., a phosphate group), or a peptidase cleavable linker (e.g., a peptide bond).

In other embodiments, at least one of the linkers (tethers) is a bio-clevable linker selected from the group consisting of DNA, RNA, disulfide, amide, peptide, functionalized monosaccharides or oligosaccharides of galactosamine, glucosamine, glucose, galactose, mannose, and combinations thereof.

In certain embodiments, the one or more C₂₂ hydrocarbon chains is conjugated to the dsRNA agent via a carrier that replaces one or more nucleotide(s). The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the dsRNA agent.

In some embodiments, the sense and antisense strands of the dsRNA agent are each 15 to 30 nucleotides in length.

In one embodiment, the sense and antisense strands of a dsRNA agent are each 19 to 25 nucleotides in length.

In one embodiment, the sense and antisense strands of the dsRNA agent are each 21 to 23 nucleotides in length.

In some embodiments, the dsRNA agent comprises a single-stranded overhang on at least one of the termini, e.g., 3′ and/or 5′ overhang(s) of 1-10 nucleotides in length, for instance, an overhang of 1, 2, 3, 4, 5, or 6 nucleotides. In some embodiments, both strands have at least one stretch of 1-5 (e.g., 1, 2, 3, 4, or 5) single-stranded nucleotides in the double stranded region. In one embodiment, the single-stranded overhang is 1, 2, or 3 nucleotides in length. In some embodiments, the dsRNA agent may also have a blunt end, located at the 5′-end of the antisense strand (or the 3′-end of the sense strand), or vice versa. In one embodiment, the dsRNA agent comprises a 3′ overhang at the 3′-end of the antisense strand, and optionally a blunt end at the 5′-end of the antisense strand. In one embodiment, the dsRNA agent has a 5′ overhang at the 5′-end of the sense strand, and optionally a blunt end at the 5′-end of the antisense strand. In one embodiment, the dsRNA agent has two blunt ends at both ends of the iRNA duplex.

In some embodiments, at least one end of the dsRNA agent is blunt-ended.

In one embodiment, the sense strand of the dsRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, the lipophilic moiety is conjugated to a nucleobase, sugar moiety, or internucleosidic phosphate linkage of the dsRNA agent.

In some embodiments, the dsRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the dsRNA agent does not contain a 5′-vinyl phosphonate (VP).

In some embodiments, the dsRNA agent further comprises at least one terminal, chiral phosphorus atom.

A site specific, chiral modification to the internucleotide linkage may occur at the 5′ end, 3′ end, or both the 5′ end and 3′ end of a strand. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a strand. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in PCT/US18/67103, entitled “Chirally-Modified Double-Stranded RNA Agents,” filed Dec. 21, 2018, which is incorporated herein by reference in its entirety.

In some embodiments, the dsRNA agent further comprises a terminal, chiral modification occurring at the first internucleotide linkage at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp configuration or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first, second, and third internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the third internucleotide linkages at the 3′ end of the antisense strand, having the linkage phosphorus atom in Rp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the first, and second internucleotide linkages at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In some embodiments, the dsRNA agent has at least two phosphorothioate internucleotide linkages at the first five nucleotides on the antisense strand (counting from the 5′ end).

In some embodiments, the antisense strand comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In some embodiments, the dsRNA agent further comprises a targeting ligand that targets a receptor which mediates delivery to a skeletal muscle, cardiac muscle, or adipose tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, LDL receptor ligand, trans-retinol, RGD peptide, LDL receptor ligand, CD63 ligand, CD36, and carbohydrate based ligand.

In some embodiments, the dsRNA agent further comprises a targeting ligand that targets a liver tissue. In some embodiments, the targeting ligand is a carbohydrate-based ligand. In one embodiment, the targeting ligand is a GalNAc conjugate.

In some embodiments, the dsRNA agent further comprises a dual targeting ligand that targets a liver tissue and a receptor which mediates delivery to a skeletal muscle, cardiac muscle, or adipose tissue.

All the above aspects and embodiments would be applicable to an oligonucleotide having one or more C₂₂ hydrocarbon chains conjugated to one or more internal positions on the oligonucleotide. In some embodiments, 100%, 95%, 90%, 85%, 80%, 75%, 70%, 65%, 60%, 55%, 50%, 45%, 40%, 35% or 30% of the oligonucleotide is modified. For example, when 50% of the oligonucleotide is modified, 50% of all nucleotides present in the oligonucleotide contain a modification as described herein.

In one embodiment, the oligonucleotide is a double-stranded dsRNA agent, and at least 50% of the nucleotides of the double-stranded dsRNA agent is independently modified with 2′-O-methyl, 2′-O-allyl, 2′-deoxy, or 2′-fluoro.

In one embodiment, the oligonucleotide is an antisense oligonucleotide, and at least 50% of the nucleotides of the antisense oligonucleotide are independently modified with LNA, CeNA, 2′-methoxyethyl, or 2′-deoxy.

In some embodiments, the dsRNA agent has less than 12, less than 10, less than 8, less than 6, less than 4, less than 2, or no 2′-F modifications on the sense strand. In some embodiments, the dsRNA agent has less than 12, less than 10, less than 8, less than 6, less than 4, less than 2, or no 2′-F modifications on the antisense strand.

In some embodiments, the dsRNA agent has one or more 2′-F modifications on any position of the sense strand or antisense strand.

In some embodiments, the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In one embodiment, the target gene is selected from the group consisting of adrenoceptor beta 1 (ADRB1); calcium voltage-gated channel subunit alpha1 C (CACNA1C); calcium voltage-gated channel subunit alpha1 G (CACNA1G) (T type calcium channel); angiotensin II receptor type 1(AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); or Phosphodiesterase 1 (PDE1).

In one embodiment, the antisense strand comprises at least 15, e.g., 15, 16, 17, 18, 19, 20, 21, 22, or 23, contiguous nucleotides differing by no more than 3, e.g., 3, 2, 1, or 0, nucleotides from any one of the antisense nucleotide sequences in any one of Tables 2-5, 7B, 7C, 9-16, 19-26, or 28-35.

In one embodiment, the dsRNA agent is any one of the agents in any one of Tables 2, 3, 4, 5, 7B, 7C, 9-16, 19-26, or 28-35.

In one embodiment, the target gene is selected from the group consisting of Delta 4-Desaturase, Sphingolipid 1 (DEGS1); leptin; folliculin (FLCN); Zinc Finger Protein 423 (ZFP423); Cyclin Dependent Kinase 6 (CDK6); Regulatory Associated Protein Of MTOR Complex 1 (RPTOR); Mechanistic Target Of Rapamycin Kinase, (mTOR); Forkhead Box P1 (FOXP1); Phosphodiesterase 3B (PDE3B); and Activin A Receptor Type 1C (ACVR1C).

In one embodiment, the target gene is selected from the group consisting of myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); and Double Homeobox 4 (DUX4).

The present invention also provides cells and pharmaceutical compositions comprising the dsRNA agents of the invention. In another aspect, the present invention provides a method of inhibiting expression of a target gene in a skeletal muscle cell, a cardiac muscle cell, or an adipocyte, or adipose tissue. The method includes contacting the cell with a dsRNA agent that inhibits expression of a target gene, wherein the dsRNA agent comprises an antisense strand which is complementary to the target gene; a sense strand which is complementary to the antisense strand and forms a double stranded region with the antisense strand; and one or more C₂₂ hydrocarbon chains conjugated to one or more internal positions on at least one strand, wherein the dsRNA agent is suitable for delivery to a muscle tissue, e.g., skeletal muscle tissue or cardiac muscle tissue, or an adipose tissue. In some embodiments, the one or more C₂₂ hydrocarbon chains conjugated to one or more internal positions on at least one strand are conjugated to the dsRNA agent via a linker or carrier.

In some embodiments, the lipophilicity of the one or more C₂₂ hydrocarbon chain, measured by octanol-water partition coefficient, log K_{ow}, exceeds 0. The lipophilic moiety may possess a log K_{ow} exceeding 1, exceeding 1.5, exceeding 2, exceeding 3, exceeding 4, exceeding 5, or exceeding 10.

In some embodiments, the hydrophobicity of the dsRNA agent, measured by the unbound fraction in the plasma protein binding assay of the dsRNA agent, exceeds 0.2. In one embodiment, the plasma protein binding assay determined is an electrophoretic mobility shift assay (EMSA) using human serum albumin protein. The hydrophobicity of the dsRNA agent, measured by fraction of unbound dsRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of dsRNA/

The C₂₂ hydrocarbon chain may be saturated or unsaturated.

The C₂₂ hydrocarbon chain may be linear or branched

In some embodiments, the internal positions include all positions except the three terminal positions from each end of the at least one strand.

In some embodiments, the internal positions exclude a cleavage site region of the sense strand.

In some embodiments, the internal positions exclude positions 9-12 or positions 11-13, counting from the 5′-end of the sense strand.

In some embodiments, the internal positions exclude a cleavage site region of the antisense strand.

In some embodiments, the internal positions exclude positions 12-14, counting from the 5′-end of the antisense strand.

In some embodiments, the one or more C₂₂ hydrocarbon chains are conjugated to one or more of the following internal positions: positions 4-8 and 13-18 on the sense strand, and positions 6-10 and 15-18 on the antisense strand, counting from the 5′ end of each strand.

In some embodiments, the one or more C₂₂ hydrocarbon chains are conjugated to one or more of the following internal positions: positions 5, 6, 7, 15, and 17 on the sense strand, and positions 15 and 17 on the antisense strand, counting from the 5′-end of each strand.

In some embodiments, the one or more C₂₂ hydrocarbon chains are conjugated to position 6 on the sense strand, counting from the 5′-end of the sense strand.

In some embodiments, the one or more C₂₂ hydrocarbon chains is an aliphatic, alicyclic, or polyalicyclic compound, e.g., the one or more C₂₂ hydrocarbon chains contains a functional group selected from the group consisting of hydroxyl, amine, carboxylic acid, sulfonate, phosphate, thiol, azide, and alkyne.

In some embodiments, the one or more C₂₂ hydrocarbon chains is a C22 acid, e.g. the C22 acid is selected from the group consisting of docosanoic acid, 6-octyltetradecanoic acid, 10-hexylhexadecanoic acid, all-cis-7,10,13,16,19-docosapentaenoic acid, all-cis-4,7,10,13,16,19-docosahexaenoic acid, all-cis-13,16-docosadienoic acid, all-cis-7,10,13,16-docosatetraenoic acid, all-cis-4,7,10,13,16-docosapentaenoic acid, and cis-13-docosenoic acid.

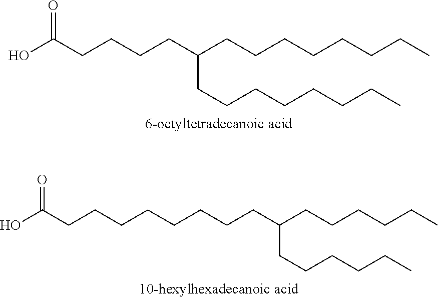 [SMILES: CCCCCCCCC(CCCCCCCC)CCCCC(=O)O ; CCCCCCC(CCCCCC)CCCCCCCCC(=O)O]

In some embodiments, the one or more C₂₂ hydrocarbon chains is a C22 alcohol, e.g., the C22 alcohol is selected from the group consisting of 1-docosanol, 6-octyltetradecan-1-ol, 10-hexylhexadecan-1-ol, cis-13-docosen-1-ol, docosan-9-ol, docosan-2-ol, docosan-10-ol, docosan-11-ol, and cis-4,7,10,13,16,19-docosahexanol.

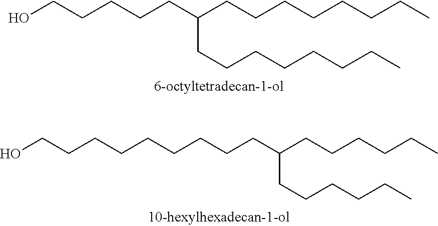 [SMILES: CCCCCCCCC(CCCCCO)CCCCCCCC ; CCCCCCC(CCCCCC)CCCCCCCCCO]

In some embodiments, the one or more C₂₂ hydrocarbon chains is a C22 amide, e.g., the C22 amide is selected from the group consisting of (E)-Docos-4-enamide, (E)-Docos-5-enamide, (Z)-Docos-9-enamide, (E)-Docos-11-enamide, 12-Docosenamide, (Z)-Docos-13-enamide, (Z)—N-Hydroxy-13-docoseneamide, (E)-Docos-14-enamide, 6-cis-Docosenamide, 14-Docosenamide Docos-11-enamide, (4E, 13E)-Docosa-4,13-dienamide, and (5E, 13E)-Docosa-5,13-dienamide.

The one or more C₂₂ hydrocarbon chains may be conjugated to the dsRNA agent via a direct attachment to the ribosugar of the dsRNA agent. Alternatively, the one or more C₂₂ hydrocarbon chains may be conjugated to the dsRNA agent via a linker or a carrier. In some embodiments, the one or more C₂₂ hydrocarbon chains may be conjugated to the dsRNA agent via internucleotide phosphate linkage.

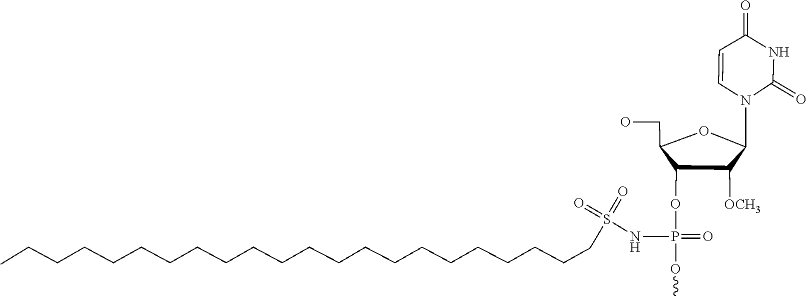

In certain embodiments, the one or more C₂₂ hydrocarbon chains is conjugated to the dsRNA agent via one or more linkers (tethers), e.g., a carrier that replaces one or more nucleotide(s) in the internal position(s).

In some embodiments, the one or more C₂₂ hydrocarbon chains is conjugated to the dsRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, at least one of the linkers (tethers) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., a phosphate group), or a peptidase cleavable linker (e.g., a peptide bond).

In other embodiments, at least one of the linkers (tethers) is a bio-clevable linker selected from the group consisting of DNA, RNA, disulfide, amide, peptide, e.g., protease cleavable peptide, functionalized monosaccharides or oligosaccharides of galactosamine, glucosamine, glucose, galactose, mannose, and combinations thereof.

In certain embodiments, the one or more C₂₂ hydrocarbon chains is conjugated to the dsRNA agent via a carrier that replaces one or more nucleotide(s). The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the dsRNA agent.

In some embodiments, the sense and antisense strands of the dsRNA agent are each 15 to 30 nucleotides in length.

In one embodiment, the sense and antisense strands of a dsRNA agent are each 19 to 25 nucleotides in length.

In one embodiment, the sense and antisense strands of the dsRNA agent are each 21 to 23 nucleotides in length.

In some embodiments, the dsRNA agent comprises a single-stranded overhang on at least one of the termini, e.g., 3′ and/or 5′ overhang(s) of 1-10 nucleotides in length, for instance, an overhang of 1, 2, 3, 4, 5, or 6 nucleotides. In some embodiments, both strands have at least one stretch of 1-5 (e.g., 1, 2, 3, 4, or 5) single-stranded nucleotides in the double stranded region. In one embodiment, the single-stranded overhang is 1, 2, or 3 nucleotides in length. In some embodiments, the dsRNA agent may also have a blunt end, located at the 5′-end of the antisense strand (or the 3′-end of the sense strand), or vice versa. In one embodiment, the dsRNA agent comprises a 3′ overhang at the 3′-end of the antisense strand, and optionally a blunt end at the 5′-end of the antisense strand. In one embodiment, the dsRNA agent has a 5′ overhang at the 5′-end of the sense strand, and optionally a blunt end at the 5′-end of the antisense strand. In one embodiment, the dsRNA agent has two blunt ends at both ends of the iRNA duplex.

In some embodiments, at least one end of the dsRNA agent is blunt-ended.

In one embodiment, the sense strand of the dsRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, the lipophilic moiety is conjugated to a nucleobase, sugar moiety, or internucleosidic phosphate linkage of the dsRNA agent.

In some embodiments, the dsRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the dsRNA agent does not contain a 5′-vinyl phosphonate (VP).

In some embodiments, the dsRNA agent further comprises at least one terminal, chiral phosphorus atom.

A site specific, chiral modification to the internucleotide linkage may occur at the 5′ end, 3′ end, or both the 5′ end and 3′ end of a strand. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a strand. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in PCT/US18/67103, entitled “Chirally-Modified Double-Stranded RNA Agents,” filed Dec. 21, 2018, which is incorporated herein by reference in its entirety.

In some embodiments, the dsRNA agent further comprises a terminal, chiral modification occurring at the first internucleotide linkage at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp configuration or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first, second, and third internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the third internucleotide linkages at the 3′ end of the antisense strand, having the linkage phosphorus atom in Rp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the first, and second internucleotide linkages at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In some embodiments, the dsRNA agent has at least two phosphorothioate internucleotide linkages at the first five nucleotides on the antisense strand (counting from the 5′ end).

In some embodiments, the antisense strand comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In some embodiments, the dsRNA agent further comprises a targeting ligand that targets a receptor which mediates delivery to a skeletal muscle, cardiac muscle, or adipose tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, LDL receptor ligand, trans-retinol, RGD peptide, LDL receptor ligand, CD63 ligand, CD36, and carbohydrate based ligand.

In some embodiments, the dsRNA agent further comprises a targeting ligand that targets a liver tissue. In some embodiments, the targeting ligand is a carbohydrate-based ligand. In one embodiment, the targeting ligand is a GalNAc conjugate.

In some embodiments, the dsRNA agent further comprises a dual targeting ligand that targets a liver tissue and a receptor which mediates delivery to a skeletal muscle, cardiac muscle, or adipose tissue.

All the above aspects and embodiments would be applicable to an oligonucleotide having one or more C₂₂ hydrocarbon chains conjugated to one or more internal positions on the oligonucleotide. In some embodiments, 100%, 95%, 90%, 85%, 80%, 75%, 70%, 65%, 60%, 55%, 50%, 45%, 40%, 35% or 30% of the oligonucleotide is modified. For example, when 50% of the oligonucleotide is modified, 50% of all nucleotides present in the oligonucleotide contain a modification as described herein.

In one embodiment, the oligonucleotide is a double-stranded dsRNA agent, and at least 50% of the nucleotides of the double-stranded dsRNA agent is independently modified with 2′-O-methyl, 2′-O-allyl, 2′-deoxy, or 2′-fluoro.

In one embodiment, the oligonucleotide is an antisense oligonucleotide, and at least 50% of the nucleotides of the antisense oligonucleotide are independently modified with LNA, CeNA, 2′-methoxyethyl, or 2′-deoxy.

In some embodiments, the dsRNA agent has less than 12, less than 10, less than 8, less than 6, less than 4, less than 2, or no 2′-F modifications on the sense strand. In some embodiments, the dsRNA agent has less than 12, less than 10, less than 8, less than 6, less than 4, less than 2, or no 2′-F modifications on the antisense strand.

In some embodiments, the dsRNA agent has one or more 2′-F modifications on any position of the sense strand or antisense strand.

In some embodiments, the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In one embodiment, the target gene is selected from the group consisting of adrenoceptor beta 1 (ADRB1); calcium voltage-gated channel subunit alpha1 C (CACNA1C); calcium voltage-gated channel subunit alpha1 G (CACNA1G) (T type calcium channel); angiotensin II receptor type 1(AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); or Phosphodiesterase 1 (PDE1).

In one embodiment, the antisense strand comprises at least 15, e.g., 15, 16, 17, 18, 19, 20, 21, 22, or 23, contiguous nucleotides differing by no more than 3, e.g., 3, 2, 1, or 0, nucleotides from any one of the antisense nucleotide sequences in any one of Tables 2-5, 7B, 7C, 9-16, 19-26, and 28-35.

In one embodiment, the dsRNA agent is any one of the agents in any one of Tables 2, 3, 4, 5, 7B, 7C, 9-16, 19-26, or 28-35.

In one embodiment, the target gene is selected from the group consisting of Delta 4-Desaturase, Sphingolipid 1 (DEGS1); leptin; folliculin (FLCN); Zinc Finger Protein 423 (ZFP423); Cyclin Dependent Kinase 6 (CDK6); Regulatory Associated Protein Of MTOR Complex 1 (RPTOR); Mechanistic Target Of Rapamycin Kinase, (mTOR); Forkhead Box P1 (FOXP1); Phosphodiesterase 3B (PDE3B); and Activin A Receptor Type 1C (ACVR1C).

In another embodiment, the target gene is selected from the group consisting of PPARG, ADIPOQ, CD36, LPL, ADAMTS9, RASD1, GYS2, CAT, DPYS, MLXIPL, VEGFA, HLA-DQA1, LIPA, CTSC, FCGR2A, GBE1, SH2B3, CTSK, CDKN2B, ELN, ARG1, HHEX, TCF7L2, CYP2A6, ALDH2, ACADS, GLYCTK, LDLR, HAL, ACER3, SLC7A7. PTPN22, CDKN1C, LEPR, SNAI2, PGM1, IGF2BP2, TTPA, ATP7B, ASPA, ADRB3, MAN2B1, RCAN1, PIGL, TBX1, LMNB1, FBP1, ETFA, LMNA, LAT2, PRKAG2, SELENBP1, TKT, PCSK1, PSAP, NDN, ACYl, SATB2, CYP21A2, POMC, CDC73, CTSH, CFTR, CTSA, G6PD, EXT1, EXT2, CPT1A, SEMA5A, WFS1, KIT, ACAT1, GGCX, FKBP6, PPARGC1B, DGCR6, HMGCS2, PEPD, WRN, LCAT, KLF13, SLC16A2, DHCR7, ITPR3, CLDN4, FZD9, SLC30A2, APOA5, HADHA, CDKAL1, PTPN2, LIPC, CD226, PON1, MCCC1, EIF2AK3, GYG1, BCL7B, AGL, VKORC1, BAZ1B, NAGS, ASL, STAR, ACP2, POLG, GAA, ALDH3A2, MANBA, ARSA, AGA, CYP27B1, CPS1, DLAT, DCXR, EIF4H, DYRKIA, GTF2I, LAMP2, CTH, EPO, FLAD1, AKT2, WAC, GLB1, RFC2, BACH2, D2HGDH, GHRL, TBL2, RRM2B, PRKACA, DLD, NEU1, ADSL, SLC22A5, ADCY10, INSR, HSD17B10, DGCR8, NPAP1, OXCT1, SDC3, HMGCL, PGAP1, MCCC2, LMF1, PIGM, UCP3, PAH, VPS33A, BCS1L, PDP1, AHCY, ALDH18A1, ENO3, MTTP, MAT1A, GNPTAB, PHGDH, BCAT2, CBS, HDAC4, LIG3, PSAT1, HGD, CTNND2, PDHB, PDHA1, NADK2, UPB1, PKLR, BCKDK, MEN1, GALT, LIMK1, SLC39A4, KCNJ11, PDHX, ACAD8, GSS, CHRNA7, SLC6A9, ERBB3, GLUD1, GSR, OAT, SLC6A8, CLIP2, STX1A, CARTPT, SLC25A15, DGCR2, LIPT2, NR5A1, DNM1L, PHEX, SLC30A9, B3GAT3, SLC34A3, SLC12A3, EPX, SARS2, CAPN10, ASNS, ALDOB, AGRP, MFF, GK, APOC2, CLDN3, HPRT1, PFKM, AMACR, SNRPN, HNF1B, L2HGDH, SORD, IDH2, TPMT, CYP2C19, TERT, MC4R, TMPRSS15, SLCO1B3, FGF23, PAX4, SLC30A8, MTNR1B, SI, SLCO1B1, and NROB2.

In one embodiment, the target gene is selected from the group consisting of myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); and Double Homeobox 4 (DUX4).

In one embodiment, the cell is within a subject.

In one embodiment, the subject is a human.

In another aspect, the present invention provides a method of treating a subject having a skeletal muscle disorder, a cardiac muscle disorder, or an adipose tissue disorder, comprising administering to the subject a therapeutically effective amount of the dsRNA agent of the invention or a pharmaceutical composition of the invention, thereby treating the subject.

In one embodiment, the cardiac muscle disorder is selected from the group consisting of obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); and hypertrophic cardiomyopathy (HCM).

In one embodiment, the skeletal muscle disorder is selected from the group consisting of Myostatin-related muscle hypertrophy, congenital myasthenic syndrome, and facioscapulohumeral muscular dystrophy (FSHD).

In one embodiment, the adipose tissue disorder is selected from the group consisting of a metabolic disorder, e.g. metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

The dsRNA agent may be administered to the subject intravenously, subcutaneously or intramuscularly.

In one embodiment, the dsRNA agent is administered to the subject intramuscularly.

In one embodiment, the dsRNA agent is administered to the subject subcutaneously.

In one embodiment, the methods of the invention further include administering to the subject an additional agent or a therapy suitable for treatment or prevention of a skeletal muscle disorder, cardiac muscle disorder, or an adipose tissue disorder.

In one aspect, the present invention provides an RNA-induced silencing complex (RISC) comprising an antisense strand of any of the dsRNA agents of the invention.

In another embodiment, the RNAi agent is a pharmaceutically acceptable salt thereof. “Pharmaceutically acceptable salts” of each of RNAi agents herein include, but are not limited to, a sodium salt, a calcium salt, a lithium salt, a potassium salt, an ammonium salt, a magnesium salt, an mixtures thereof. One skilled in the art will appreciate that the RNAi agent, when provided as a polycationic salt having one cation per free acid group of the optionally modified phosophodiester backbone and/or any other acidic modifications (e.g., 5′-terminal phosphonate groups). For example, an oligonucleotide of “n” nucleotides in length contains n-1 optionally modified phosophodiesters, so that an oligonucleotide of 21 nt in length may be provided as a salt having up to 20 cations (e.g, 20 sodium cations). Similarly, an RNAi agentshaving a sense strand of 21 nt in length and an antisense strand of 23 nt in length may be provided as a salt having up to 42 cations (e.g, 42 sodium cations). In the preceding example, where the RNAi agent also includes a 5′-terminal phosphate or a 5′-terminal vinylphosphonate group, the RNAi agent may be provided as a salt having up to 44 cations (e.g, 44 sodium cations).

In one aspect, the present invention provides a method of synthesizing a nucleoside monomer having the structure of Formula (I):

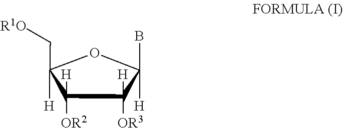

wherein:
B is a modified or unmodified nucleobase;
R¹ is a hydroxyl protecting group;
R² is H or phosphoramidite;
R³ is C₂₂H₄₅
   wherein the compound of formula (I) is free or substantially free of a compound of Formula (II)

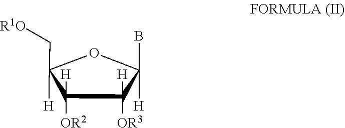

wherein:
B is a modified or unmodified nucleobase;
R¹ is a hydroxyl protecting group;
R² is C₂₂H₄₅
R³ is H

In one embodiment, the hydroxyl protecting group is selected from the group consisting of 4,4′-dimethoxytrityl (DMT), monomethoxytrityl (MMT), 9-fluorenlnethylcarbonate (Fnoc), o-nitrophenylcarbonyl, p-phenylazopheniylcarbonlk, phenlcarbony, p-chlorophenylcarbonyl, and 5′-(α-methyl-2-lnitropiperonyl)oxycartonyl (MeNPOC).

### BRIEF DESCRIPTION OF THE DRAWINGS

FIG. 1 is a graph depicting the effect of intravenous administration of a single 5 mg/kg or 20 mg/kg dose of the indicated dsRNA agents at Day 14 post-dose on SOD1 mRNA expression in liver, cardiac, and skeletal muscle tissues in mice.

FIG. 2A is a graph depicting the effect of intravenous administration of a single 1 mg/kg, 5 mg/kg, or 20 mg/kg dose of duplex AD-1615344 at Day 14 and Day 28 post-dose on MALAT1 mRNA expression in skeletal muscle tissue in mice.

FIG. 2B is a graph depicting the effect of intravenous administration of a single 1 mg/kg, 5 mg/kg, or 20 mg/kg dose of duplex AD-1615345 at Day 14 and Day 28 post-dose on MALAT1 mRNA expression in skeletal muscle tissue in mice.

FIG. 3A is a graph depicting the effect of route of administration of a single 5 mg/kg dose of AD-1427062, targeting the mouse SOD1 gene, on SOD1 mRNA expression in skeletal muscle tissue. IV, intravenous administration; IM(I), intramuscular administration of AD-1427062; IM(D); intramuscular administration of distal skeletal muscle with PBS control; SQ, subcutaneous administration; and IP, intraperitoneal administration.

FIG. 3B is a graph depicting the effect of route of administration of a single 5 mg/kg dose of AD-1427062, targeting the mouse SOD1 gene, on SOD1 mRNA expression in cardiac muscle tissue. IV, intravenous administration; IM(I), intramuscular administration; SQ, subcutaneous administration; and IP, intraperitoneal administration.

FIG. 3C is a graph depicting the effect of route of administration of a single 5 mg/kg dose of AD-1640773, targeting the mouse MSTN1 gene, on SOD1 mRNA expression in skeletal muscle tissue. IV, intravenous administration; IM(I), intramuscular administration of AD-1640773; IM(D); intramuscular administration of distal skeletal muscle with PBS control; and SQ, subcutaneous administration.

FIG. 3D is a graph depicting the effect of route of administration of a single 5 mg/kg dose of AD-1427062, targeting the mouse SOD1 gene, on SOD1 mRNA expression in adipose tissue. GAPDH mRNA expression was used as a control. IV, intravenous administration; IM(I), intramuscular administration; SQ, subcutaneous administration; and IP, intraperitoneal administration.

FIG. 3E is a graph depicting the effect of route of administration of a single 5 mg/kg dose of AD-1427062, targeting the mouse SOD1 gene, on SOD1 mRNA expression in adipose tissue. HPRT mRNA expression was used as a control. IV, intravenous administration; IM(I), intramuscular administration; SQ, subcutaneous administration; and IP, intraperitoneal administration.

FIG. 4A is a graph depicting SOD1 mRNA silencing in mouse gonadal adipose tissue at Days 14 and 28 post-dose of a single intravenously administered 0.5 mg/kg, 2 mg/kg or 5 mg/kg dose of a dsRNA agent targeting SOD1. GAPDH mRNA expression was used as a control.

FIG. 4B is a graph depicting SOD1 mRNA silencing in mouse subcutaneous adipose tissue at Days 14 and 28 post-dose of a single intravenously administered 0.5 mg/kg, 2 mg/kg or 5 mg/kg dose of a dsRNA agent targeting SOD1. GAPDH mRNA expression was used as a control.

FIG. 4C is a graph depicting SOD1 mRNA silencing in mouse brown intrascapular adipose tissue at Days 14 and 28 post-dose of a single intravenously administered 0.5 mg/kg, 2 mg/kg or 5 mg/kg dose of a dsRNA agent targeting SOD1. GAPDH mRNA expression was used as a control.

FIG. 5 is a graph summarizing SOD1 mRNA silencing in non-human primates' adipose tissue (brown adipose, white hind limb, white subcutaneous and white uterine) at 30 days post-dose of a single 3 mg/kg IV administered dose of a dsRNA agent targeting SOD1.

FIG. 6A is a graph depicting Leptin serum concentrations in lean female mice pre-dose (Day 0) and Days 8, 14, and 21 post-dose following subcutaneous administration of a single 5 mg/kg dose of AD-1888031 or AD-1888032. Each bar represents the mean serum concentration for each treatment group at the indicated timepoint +/− standard error (n=3) compared to PBS control.

FIG. 6B is a graph depicting the percent change in leptin concentration in lean female mice pre-dose (Day 0) and Days 8, 14, and 21 post-dose following subcutaneous administration of a single 5 mg/kg dose of AD-1888031 or AD-1888032. Each bar represents the mean percent change in serum concentration for each treatment group at the indicated timepoint +/− standard error (n=3) compared to PBS control.

FIG. 7A is a graph depicting Leptin serum concentrations in lean male mice pre-dose (Day 0) and Days 8, 14, and 21 post-dose following subcutaneous administration of a single 5 mg/kg dose of AD-1888031 or AD-1888032. Each bar represents the mean serum concentration for each treatment group at the indicated timepoint +/− standard error (n=3) compared to PBS control.

FIG. 7B is a graph the percent change in leptin concentration in lean male mice pre-dose (Day 0) and Days 8, 14, and 21 post-dose following subcutaneous administration of a single 5 mg/kg dose of AD-1888031 or AD-1888032. Each bar represents the mean percent change in serum concentration for each treatment group at the indicated timepoint +/− standard error (n=3) compared to PBS control.

FIG. 8A is a graph depicting Leptin serum concentrations in high-fat diet (HFD) fed male mice pre-dose (Day 0) and Days 8, 14, and 21 post-dose following subcutaneous administration of a single 5 mg/kg dose of AD-1888031 or AD-1888032. Each bar represents the mean serum concentration for each treatment group at the indicated timepoint +/− standard error (n=3) compared to PBS control.

FIG. 8B is a graph depicting the percent change in leptin concentration in high-fat diet (HFD) fed male mice pre-dose (Day 0) and Days 8, 14, and 21 post-dose following subcutaneous administration of a single 5 mg/kg dose of AD-1888031 or AD-1888032. Each bar represents the mean percent change in serum concentration for each treatment group at the indicated timepoint +/− standard error (n=3) compared to PBS control.

FIG. 9A is a graph depicting the average relative mouse myostatin mRNA expression after intravenous administration of single 1, 2.5, or 5 mg/kg dose of lipid conjugated Mstn dsRNA agent at Day 14 post-dose in quadriceps as determined by qPCR.

FIG. 9B is a graph depicting the average relative mouse myostatin mRNA expression after intravenous administration of single 1, 2.5, or 5 mg/kg dose of lipid conjugated Mstn dsRNA agent at Dat 14 and 46 post-dose in quadriceps as determined by qPCR.

FIG. 10 is a graph depicting the average relative mouse SOD1 mRNA expression after intravenous administration of single 2 mg/kg dose of a lipid conjugated SOD1 dsRNA agent (AD-1427062) at Day 14 post-dose in quadriceps (left and right), gastrocnemius, and diaphragm as determined by qPCR.

FIG. 11 is a graph depicting the average relative mouse SOD1 mRNA expression following intravenous or subcutaneous administration of single 2 mg/kg or 1 mg/kg×2 doses (administered 1 week apart) of AD-1812376 on Day 14 post-dose in quadriceps as determined by qPCR.

FIG. 12 is a graph depicting the average relative mouse SOD1 mRNA expression following single 2 mg/kg or 1 mg/kg×2 doses of AD-1812376) agent after 21 days in heart administered either intravenously subcutaneously as determined by qPCR.

FIG. 13A is a graph depicting the average relative myostatin mRNA expression following intravenous administration of a single 2 mg/kg or 5 mg/kg dose of a dsRNA agent targeting Myostatin and comprising one or more C₂₂ hydrocarbon chains conjugated to position 6 on the sense strand, counting from the 5′-end of the sense strand, at Day 56 post-dose in quadriceps as determined by qPCR and compared to PBS control in non-human primates.

FIG. 13B is a graph depicting the average relative myostatin mRNA expression following intravenous administration of a single 2 mg/kg or 5 mg/kg dose of a dsRNA agent targeting Myostatin and comprising one or more C₂₂ hydrocarbon chains conjugated to position 6 on the sense strand, counting from the 5′-end of the sense strand, at Day 56 post-dose in gastrocnemius as determined qPCR and compared to PBS control in non-human primates.

FIG. 13C is a graph depicting the average relative myostatin protein expression following intravenous administration of a single 2 mg/kg or 5 mg/kg dose of a dsRNA agent targeting Myostatin and comprising one or more C₂₂ hydrocarbon chains conjugated to position 6 on the sense strand, counting from the 5′-end of the sense strand, at Day 56 post-dose in quadriceps and gastrocnemius as determined by ELISA and compared to PBS control in non-human primates.

### DETAILED DESCRIPTION OF THE INVENTION

The inventors have unexpectedly discovered, inter alia, that conjugating a C₂₂ lipophilic moiety to one or more internal positions on at least one strand of a dsRNA agent provides surprisingly efficient in vivo delivery to muscle and/or adipose tissue resulting in efficient entry and internalization of the dsRNA agent into muscle tissue, e.g., cardiac and skeletal muscle tissue, and/or adipose tissue, and surpringly good inhibition of target gene expression in muscle tissue, e.g., cardiac and skeletal muscle tissue, and/or adipose tissue.

Accordingly, in one aspect, the present invention provides a dsRNA agent comprising an antisense strand which is complementary to the target gene; a sense strand which is complementary to the antisense strand and forms a double stranded region with the antisense strand; and one or more C₂₂ hydrocarbon chains, e.g., saturated or unsaturated, conjugated to one or more internal positions on at least one strand, wherein the dsRNA agent is suitable for delivery to a muscle tissue or an adipose tissue. In some embodiments, the one or more C₂₂ hydrocarbon chains conjugated to one or more internal positions on at least one strand are conjugated to the dsRNA agent via a linker or carrier.

The following detailed description discloses how to make and use compositions containing dsRNA agents comprising one or more C₂₂ hydrocarbon chains to inhibit the expression of a target gene as well as compositions, uses, and methods for treating subjects that would benefit from inhibition and/or reduction of the expression of the target gene.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least”, “no less than”, or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a sequence and its indicated site on a transcript or other sequence, the nucleotide sequence recited in the specification takes precedence.

As used herein, “target sequence” or “target nucleic acid” refers to a contiguous portion of the nucleotide sequence of an mRNA molecule formed during the transcription of a target gene, including mRNA that is a product of RNA processing of a primary transcription product. In one embodment, the target portion of the sequence will be at least long enough to serve as a substrate for RNAi-directed cleavage at or near that portion of the nucleotide sequence of an mRNA molecule formed during the transcription of a target gene. In one embodiment, the target sequence is within the protein coding region of the target gene. In another embodiment, the target sequence is within the 3′ UTR of the target gene. The target nucleic acid can be a cellular gene (or mRNA transcribed from the gene) whose expression is associated with a particular disorder or disease state.

The target sequence may be from about 9-36 nucleotides in length, e.g., about 15-30 nucleotides in length. For example, the target sequence can be about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In some embodiments, the target sequence is about 19 to about 30 nucleotides in length. In other embodiments, the target sequence is about 19 to about 25 nucleotides in length. In still other embodiments, the target sequence is about 19 to about 23 nucleotides in length. In some embodiments, the target sequence is about 21 to about 23 nucleotides in length. Ranges and lengths intermediate to the above recited ranges and lengths are also contemplated to be part of the invention.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, and uracil can be replaced by other moieties without substantially altering the base pairing properties of an oligonucleotide comprising a nucleotide bearing such replacement moiety. It is understood that when a cDNA sequence is provided, the corresponding mRNA or RNAi agent would include a U in place of a T. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention. Further, one of skill in the art that a T is a target gene sequence, or reverse complement thereof, would often be replaced by a U in an RNAi agent of the invention.

The terms “iRNA”, “RNAi agent,” “iRNA agent,” “RNA interference agent” as used interchangeably herein, refer to an agent that contains RNA as that term is defined herein, and which mediates the targeted cleavage of an RNA transcript via an RNA-induced silencing complex (RISC) pathway. RNA interference (RNAi) is a process that directs the sequence-specific degradation of mRNA. RNAi modulates, e.g., inhibits, the expression of a target gene in a cell, e.g., a cell within a subject, such as a mammalian subject.

In one embodiment, an RNAi agent of the disclosure includes a single stranded RNAi that interacts with a target RNA sequence, e.g., a target mRNA sequence, to direct the cleavage of the target RNA. Without wishing to be bound by theory it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15:485). Dicer, a ribonuclease-III-like enzyme, processes these dsRNA into 19-23 base pair short interfering RNAs with characteristic two base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the disclosure relates to a single stranded RNA (ssRNA) (the antisense strand of a siRNA duplex) generated within a cell and which promotes the formation of a RISC complex to effect silencing of the target gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, an “RNAi agent” for use in the compositions and methods of the disclosure is a double stranded RNA and is referred to herein as a “double stranded RNAi agent,” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., a target mRNA sequence. In some embodiments of the disclosure, a double stranded RNA (dsRNA) triggers the degradation of a target RNA, e.g., an mRNA, through a post-transcriptional gene-silencing mechanism referred to herein as RNA interference or RNAi.

In general, a dsRNA molecule can include ribonucleotides, but as described in detail herein, each or both strands can also include one or more non-ribonucleotides, e.g., a deoxyribonucleotide, a modified nucleotide. In addition, as used in this specification, an “RNAi agent” may include ribonucleotides with chemical modifications; an RNAi agent may include substantial modifications at multiple nucleotides.

As used herein, the term “modified nucleotide” refers to a nucleotide having, independently, a modified sugar moiety, a modified internucleotide linkage, or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a siRNA type molecule, are encompassed by “RNAi agent” for the purposes of this specification and claims.

In certain embodiments of the instant disclosure, inclusion of a deoxy-nucleotide—which is acknowledged as a naturally occurring form of nucleotide—if present within a RNAi agent can be considered to constitute a modified nucleotide.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at at least 4, at least 5, at least 6, at least 7, at least 8, at least 9, at least 10, at least 20, at least 23 or more unpaired nucleotides or nucleotides not directed to the target site of the dsRNA. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

In certain embodiment, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)n; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)4, (U)4, and (dT)4, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one embodiment, an RNAi agent of the invention is a dsRNA, each strand of which is 24-30 nucleotides in length, that interacts with a target RNA sequence, e.g., a target mRNA sequence, to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188).

In one embodiment, an RNAi agent of the invention is a dsRNA agent, each strand of which comprises 19-23 nucleotides that interacts with a target mRNA sequence to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). In one embodiment, an RNAi agent of the invention is a dsRNA of 24-30 nucleotides that interacts with a target mRNA sequence to direct the cleavage of the target RNA.

As used herein, the term “nucleotide overhang” refers to at least one unpaired nucleotide that protrudes from the duplex structure of a RNAi agent, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one embodiment of the dsRNA, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In one embodiment, the antisense strand of a dsRNA has a 1-10 nucleotide, e.g., β-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′ end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′ end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double stranded over its entire length.

The term “antisense strand” or “guide strand” refers to the strand of an iRNA, e.g., a dsRNA, which includes a region that is substantially complementary to a target sequence, e.g., a target mRNA sequence.

As used herein, the term “region of complementarity” refers to the region on the antisense strand that is substantially complementary to a sequence, for example a target sequence, e.g., a target nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- or 3′-terminus of the RNAi agent.

In some embodiments, a double stranded RNA agent of the invention includes a nucleotide mismatch in the antisense strand. In some embodiments, the antisense strand of the double stranded RNA agent of the invention includes no more than 4 mismatches with the target mRNA, e.g., the antisense strand includes 4, 3, 2, 1, or 0 mismatches with the target mRNA. In some embodiments, the antisense strand double stranded RNA agent of the invention includes no more than 4 mismatches with the sense strand, e.g., the antisense strand includes 4, 3, 2, 1, or 0 mismatches with the sense strand. In some embodiments, a double stranded RNA agent of the invention includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent as described herein can contain one or more mismatches to the target sequence. In one embodiment, a RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a target gene, generally does not contain any mismatch within the central 13 nucleotides. The methods described herein or methods known in the art can be used to determine whether an RNAi agent containing a mismatch to a target sequence is effective in inhibiting the expression of a target gene. Consideration of the efficacy of RNAi agents with mismatches in inhibiting expression of a target gene is important, especially if the particular region of complementarity in a target gene is known to vary.

The term “sense strand” or “passenger strand” as used herein, refers to the strand of a RNAi agent that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can be, for example, “stringent conditions”, where stringent conditions can include: 400 mM NaCl, 40 mM PIPES pH 6.4, 1 mM EDTA, 50° C. or 70 oC for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNAi agent, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression, in vitro or in vivo. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

The terms “complementary,” “fully complementary” and “substantially complementary” herein can be used with respect to the base matching between the sense strand and the antisense strand of a dsRNA, or between two oligonucleotides or polynucleotides, such as the antisense strand of a RNAi agent and a target sequence, as will be understood from the context of their use.

As used herein, a polynucleotide that is “substantially complementary to at least part of” a messenger RNA (mRNA) or target sequence refers to a polynucleotide that is substantially complementary to a contiguous portion of the mRNA of interest or target sequence (e.g., an mRNA encoding a target gene). For example, a polynucleotide is complementary to at least a part of a target RNA if the sequence is substantially complementary to a non-interrupted portion of an mRNA encoding a target gene.

Accordingly, in some embodiments, the antisense strand polynucleotides disclosed herein are fully complementary to the target gene sequence.

Exemplary target genes include, for example, adrenoceptor beta 1 (ADRB1); calcium voltage-gated channel subunit alpha1 C (CACNA1C); calcium voltage-gated channel subunit alpha1 G (CACNA1G) (T type calcium channel); angiotensin II receptor type 1(AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); Phosphodiesterase 1 (PDE1); myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); Double Homeobox 4 (DUX4); Delta 4-Desaturase, Sphingolipid 1 (DEGS1); leptin; folliculin (FLCN); Zinc Finger Protein 423 (ZFP423); Cyclin Dependent Kinase 6 (CDK6); Regulatory Associated Protein Of MTOR Complex 1 (RPTOR); Mechanistic Target Of Rapamycin Kinase, (mTOR); Forkhead Box P1 (FOXP1); Phosphodiesterase 3B (PDE3B); and Activin A Receptor Type 1C (ACVR1C).

As used herein, “adrenoceptor beta 1,” used interchangeably with the term “ADRB1,” refers to a member of the adrenergic receptor family. The adrenergic receptors are a prototypic family of guanine nucleotide binding regulatory protein-coupled receptors that mediate the physiological effects of the hormone epinephrine and the neurotransmitter norepinephrine. Beta-1 adrenoceptors are predominately located in the heart. Specific polymorphisms in this gene have been shown to affect the resting heart rate and can be involved in heart failure. ADRB1 is also known as ADRB1R, beta-1 adrenergic receptor, B1AR, BETA1AR, FNSS2, or RHR

An exemplary sequence of a human ADRB1 mRNA transcript can be found at, for example, GenBank Accession No. GI: 1653960731 (NM_000684.3; SEQ ID NO:1; reverse complement, SEQ ID NO: 5). The sequence of mouse ADRB1 mRNA can be found at, for example, GenBank Accession No. GI: 1693744501 (NM_007419.3; SEQ ID NO:2; reverse complement, SEQ ID NO: 6). The sequence of rat ADRB1 mRNA can be found at, for example, GenBank Accession No. GI: 6978458 (NM_012701.1; SEQ ID NO:3; reverse complement, SEQ ID NO: 7). The sequence of Macaca mulatta ADRB1 mRNA can be found at, for example, GenBank Accession No. GI: 577861029 (NM_001289866.1; SEQ ID NO: 4; reverse complement, SEQ ID NO: 8). The sequence of Macaca fascicularis ADRB1 mRNA can be found at, for example, GenBank Accession No. GI: 985482105 (NM_001319353.1; SEQ ID NO: 9; reverse complement, SEQ ID NO: 10).

Additional examples of ADRB1 mRNA sequences are readily available through publicly available databases, e.g., GenBank, UniProt, OMIM, UCSC Genome Browser, and the Macaca genome project web site.

Further information on ADRB1 can be found, for example, at www.ncbi.nlm.nih.gov/gene/!term=ADRB1.

The entire contents of each of the foregoing GenBank Accession numbers and the Gene database numbers are incorporated herein by reference as of the date of filing this application.

The term ADRB1, as used herein, also refers to variations of the ADRB1 gene including variants provided in the SNP database. Numerous sequence variations within the ADRB1 gene have been identified and may be found at, for example, NCBI dbSNP and UniProt (see, e.g., www.ncbi.nlm.nih.gov/snp/!term=ADRB1, the entire contents of which is incorporated herein by reference as of the date of filing this application.

In one embodiment, the target gene is calcium voltage-gated channel subunit alpha1 C (CACNA1C).

As used herein, “calcium voltage-gated channel subunit alpha1 C,” used interchangeably with the term “CACNA1C,” refers to an alpha-1 subunit of a voltage-dependent calcium channel. Calcium channels mediate the influx of calcium ions into the cell upon membrane polarization. The alpha-1 subunit consists of 24 transmembrane segments and forms the pore through which ions pass into the cell. The calcium channel consists of a complex of alpha-1, alpha-2/delta, beta, and gamma subunits in a 1:1:1:1 ratio. There are multiple isoforms of each of these proteins, either encoded by different genes or the result of alternative splicing of transcripts. The protein encoded by this gene binds to and is inhibited by dihydropyridine. CACNA1C is also known as calcium channel, voltage-dependent, L type, alpha 1C subunit; voltage-dependent L-type calcium channel subunit alpha-1C; voltage-gated L-type calcium channel Cav1.2 alpha 1 subunit, splice variant 10; calcium channel, L type, alpha-1 polypeptide, isoform 1, cardiac muscle; calcium channel, cardic dihydropyridine-sensitive, alpha-1 subunit; voltage-dependent L-type Ca2+ channel alpha 1 subunit; voltage-gated calcium channel subunit alpha CaV1.2; DHPR, alpha-1 subunit; CACH2, CACN2, CACNL1A1, CCHL1A1, CaV1.2, LQT8, TS, or TS. LQT8

An exemplary sequence of a human CACNA1C mRNA transcript can be found at, for example, GenBank Accession No. GI: 1890333913 (NM_199460.4; SEQ ID NO: 11; reverse complement, SEQ ID NO: 12). The sequence of mouse CACNA1C mRNA can be found at, for example, GenBank Accession No. GI: 594140631 (NM_009781.4; SEQ ID NO: 13; reverse complement, SEQ ID NO: 14). The sequence of rat CACNA1C mRNA can be found at, for example, GenBank Accession No. GI: 158186632 (NM_012517.2; SEQ ID NO:15; reverse complement, SEQ ID NO: 16). The sequence of Macaca mulatta CACNA1C mRNA can be found at, for example, GenBank Accession No. GI: 1622843324 (XM_028829106.1; SEQ ID NO: 17; reverse complement, SEQ ID NO: 18).

Additional examples of CACNA1C mRNA sequences are readily available through publicly available databases, e.g., GenBank, UniProt, OMIM, UCSC Genome Browser, and the Macaca genome project web site.

Further information on CACNA1C can be found, for example, at www.ncbi.nlm.nih.gov/gene/!term=CACNA1C.

The entire contents of each of the foregoing GenBank Accession numbers and the Gene database numbers are incorporated herein by reference as of the date of filing this application.

The term CACNA1C, as used herein, also refers to variations of the CACNA1C gene including variants provided in the SNP database. Numerous sequence variations within the CACNA1C gene have been identified and may be found at, for example, NCBI dbSNP and UniProt (see, e.g., www.ncbi.nlm.nih.gov/gene/!term=CACNA1C, the entire contents of which is incorporated herein by reference as of the date of filing this application.

As used herein, “calcium voltage-gated channel subunit alpha1 G,” used interchangeably with the term “CACNA1G,” refers to a T-type, low-voltage activated calcium channel. Voltage-sensitive calcium channels mediate the entry of calcium ions into excitable cells, and are also involved in a variety of calcium-dependent processes, including muscle contraction, hormone or neurotransmitter release, gene expression, cell motility, cell division, and cell death. The T-type channels generate currents that are both transient, owing to fast inactivation, and tiny, owing to small conductance. T-type channels are thought to be involved in pacemaker activity, low-threshold calcium spikes, neuronal oscillations and resonance, and rebound burst firing. CACNA1G is also known as calcium channel, voltage-dependent, T type, alpha 1G subunit; voltage-dependent T-type calcium channel subunit alpha-1G; voltage-gated calcium channel subunit alpha Cav3.1; NBR13; Cav3.1c; Ca(V)T.1; KIAA1123; SCA42ND; or SCA42.

An exemplary sequence of a human CACNA1G mRNA transcript can be found at, for example, GenBank Accession No. GI: 1519244109 (NM_018896.5; SEQ ID NO: 21; reverse complement, SEQ ID NO: 22). The sequence of mouse CACNA1G mRNA can be found at, for example, GenBank Accession No. GI: 295444826 (NM_009783.3; SEQ ID NO: 23; reverse complement, SEQ ID NO: 24). The sequence of rat CACNA1G mRNA can be found at, for example, GenBank Accession No. GI: 1995160279 (NM_001308302.2; SEQ ID NO: 25; reverse complement, SEQ ID NO: 26). The sequence of Macaca mulatta CACNA1G mRNA can be found at, for example, GenBank Accession No. GI: 1622879013 (XM_015119270.2; SEQ ID NO: 27; reverse complement, SEQ ID NO: 28). The sequence of Macaca fascicularis CACNA1G mRNA can be found at, for example, GenBank Accession No. GI: 982305044 (XM_005583707.2; SEQ ID NO: 29; reverse complement, SEQ ID NO: 30).

Additional exemplary examples of CACNA1G mRNA sequences are readily available through publicly available databases, e.g., GenBank, UniProt, OMIM, UCSC Genome Browser, and the Macaca genome project web site.

Further information on CACNA1G can be found, for example, at www.ncbi.nlm.nih.gov/gene/!term=CACNA1G.

The entire contents of each of the foregoing GenBank Accession numbers and the Gene database numbers are incorporated herein by reference as of the date of filing this application.

The term CACNA1G, as used herein, also refers to variations of the CACNA1G gene including variants provided in the SNP database. Numerous sequence variations within the CACNA1G gene have been identified and may be found at, for example, NCBI dbSNP and UniProt (see, e.g., www.ncbi.nlm.nih.gov/gene/!term=CACNA1G, the entire contents of which is incorporated herein by reference as of the date of filing this application.

As used herein, “angiotensin II receptor type 1,” used interchangeably with the term “AGTR1,” refers to a receptor for the vasoconstricting peptide angiotensin II. Angiotensin II is a potent vasopressor hormone and a primary regulator of aldosterone secretion. AGTR1 is activated by angiotensin II. The activated receptor in turn couples to G protein and, thus, activates phospholipase C and increases the cytosolic Ca2+ concentrations, which in turn triggers cellular responses such as stimulation of protein kinase C. AGTR1 plays an integral role in blood pressure control, and is implicated in the pathogenesis of hypertension. AGTR1 is also known as angiotensin receptor 1B, AT1, AT2R1, AGTR1A, AT2R1B, AGTR1B, HAT1R, AG2S, AT1B, AT2R1A, AT1AR, AT1BR, or AT1R.

An exemplary sequence of a human AGTR1 mRNA transcript can be found at, for example, GenBank Accession No. GI: 1820101583 (NM_000685.5; SEQ ID NO: 31; reverse complement, SEQ ID NO: 32). The sequence of mouse AGTR1 mRNA can be found at, for example, GenBank Accession No. GI: 158937294 (NM_177322.3; SEQ ID NO: 33; reverse complement, SEQ ID NO: 34). The sequence of rat AGTR1 mRNA can be found at, for example, GenBank Accession No. GI: 140969764 (NM_030985.4; SEQ ID NO: 35; reverse complement, SEQ ID NO: 36). The sequence of Macaca mulatta AGTR1 mRNA can be found at, for example, GenBank Accession No. GI: 1622904093 (XM_028843763.1; SEQ ID NO: 37; reverse complement, SEQ ID NO: 38). The sequence of Macaca fascicularis AGTR1 mRNA can be found at, for example, GenBank Accession No. GI: 544411901 (XM_005546040.1; SEQ ID NO: 39; reverse complement, SEQ ID NO: 40).

Additional examples of AGTR1 mRNA sequences are readily available through publicly available databases, e.g., GenBank, UniProt, OMIM, UCSC Genome Browser, and the Macaca genome project web site.

Further information on AGTR1 can be found, for example, at www.ncbi.nlm.nih.gov/gene/!term=AGTR1.

The entire contents of each of the foregoing GenBank Accession numbers and the Gene database numbers are incorporated herein by reference as of the date of filing this application.

The term AGTR1, as used herein, also refers to variations of the AGTR1 gene including variants provided in the SNP database. Numerous sequence variations within the AGTR1 gene have been identified and may be found at, for example, NCBI dbSNP and UniProt (see, e.g., www.ncbi.nlm.nih.gov/gene/!term=AGTR1, the entire contents of which is incorporated herein by reference as of the date of filing this application.

As used herein, “Sodium Voltage-Gated Channel Alpha Subunit 2,” used interchangeably with the term “SCN2A,” refers to a member of the voltage-gated sodium channel family. Voltage-gated sodium channels are transmembrane glycoprotein complexes composed of a large alpha subunit with four repeat domains, each of which is composed of six membrane-spanning segments, and one or more regulatory beta subunits. Voltage-gated sodium channels function in the generation and propagation of action potentials in neurons and muscle. Specifically, SCN2A permits the sodium influx from the extracellular space into the cytosol after depolarization of the nerve membrane. Allelic variants of SCN2A are associated with seizure disorders and autism spectrum disorders. SCN2A is also known as Nav1.2, HBSCII, SCN2A1, SCN2A2, HBSCI, EIEE11, BFIC3, BFIS3, BFNIS, DEE11, EA9, or HBA.

An exemplary sequence of a human SCN2A mRNA transcript can be found at, for example, GenBank Accession No. GI: 1697699196 (NM_021007.3; SEQ ID NO: 41; reverse complement, SEQ ID NO: 42). The sequence of mouse SCN2A mRNA can be found at, for example, GenBank Accession No. GI: 1114439824 (NM_001099298.3; SEQ ID NO: 43; reverse complement, SEQ ID NO: 44). The sequence of rat SCN2A mRNA can be found at, for example, GenBank Accession No. GI: 1937915892 (NM 012647.2; SEQ ID NO: 45; reverse complement, SEQ ID NO: 46). The sequence of Macaca mulatta SCN2A mRNA can be found at, for example, GenBank Accession No. GI: 1622850108 (XM_001100368.4; SEQ ID NO: 47; reverse complement, SEQ ID NO: 48). The sequence of Macaca fascicularis SCN2A mRNA can be found at, for example, GenBank Accession No. GI: 544475515 (XM_005573351.1; SEQ ID NO: 49; reverse complement, SEQ ID NO: 50).

Additional examples of SCN2A mRNA sequences are readily available through publicly available databases, e.g., GenBank, UniProt, OMIM, UCSC Genome Browser, and the Macaca genome project web site.

Further information on SCN2A can be found, for example, at www.ncbi.nlm.nih.gov/gene/?term=SCN2A.

The entire contents of each of the foregoing GenBank Accession numbers and the Gene database numbers are incorporated herein by reference as of the date of filing this application.

The term SCN2A, as used herein, also refers to variations of the SCN2A gene including variants provided in the SNP database. Numerous sequence variations within the SCN2A gene have been identified and may be found at, for example, NCBI dbSNP and UniProt (see, e.g., www.ncbi.nlm.nih.gov/gene/?term=SCN2A, the entire contents of which is incorporated herein by reference as of the date of filing this application.

As used herein, “Hyperpolarization Activated Cyclic Nucleotide Gated Potassium Channel 1,” used interchangeably with the term “HCN1,” refers to a member of the hyperpolarization-activated cyclic nucleotide-gated (HCN) channel family. These channels are primarily expressed in the heart and in the central and peripheral nervous systems. HCN channels mediate rhythmic electrical activity of cardiac pacemaker cells, and in neurons play important roles in setting resting membrane potentials, dendritic integration, neuronal pacemaking, and establishing action potential threshold. The HCN1 protein can homodimerize or heterodimerize with other pore-forming subunits to form a potassium channel. HCN1 is also known as potassium channel 1, BCNG-1, HAC-2, BCNG1, Potassium/Sodium Hyperpolarization-Activated Cyclic Nucleotide-Gated Channel 1; Brain Cyclic Nucleotide-Gated Channel 1; Hyperpolarization Activated Cyclic Nucleotide-Gated Potassium Channel 1; GEFSP10, EIEE24, or DEE24.

An exemplary sequence of a human HCN1 mRNA transcript can be found at, for example, GenBank Accession No. GI: 1519313076 (NM_021072.4; SEQ ID NO: 51; reverse complement, SEQ ID NO: 52). The sequence of mouse HCN1 mRNA can be found at, for example, GenBank Accession No. GI: 283837798 (NM_010408.3; SEQ ID NO: 53; reverse complement, SEQ ID NO: 54). The sequence of rat HCN1 mRNA can be found at, for example, GenBank Accession No. GI: 2000186052 (NM_053375.2; SEQ ID NO: 55; reverse complement, SEQ ID NO: 56). The sequence of Macaca mulatta HCN1 mRNA can be found at, for example, GenBank Accession No. GI: 1622944535 (XM_015140004.2; SEQ ID NO: 57; reverse complement, SEQ ID NO: 58). The sequence of Macaca fascicularis HCN1 mRNA can be found at, for example, GenBank Accession No. GI: 982252681 (XM_005556858.2; SEQ ID NO: 59; reverse complement, SEQ ID NO: 60).

Additional examples of HCN1 mRNA sequences are readily available through publicly available databases, e.g., GenBank, UniProt, OMIM, UCSC Genome Browser, and the Macaca genome project web site.

Further information on HCN1 can be found, for example, at www.ncbi.nlm.nih.gov/gene/?term=HCN1.

The entire contents of each of the foregoing GenBank Accession numbers and the Gene database numbers are incorporated herein by reference as of the date of filing this application.

The term HCN1, as used herein, also refers to variations of the HCN1 gene including variants provided in the SNP database. Numerous sequence variations within the HCN1 gene have been identified and may be found at, for example, NCBI dbSNP and UniProt (see, e.g., www.ncbi.nlm.nih.gov/gene/?term=HCN1, the entire contents of which is incorporated herein by reference as of the date of filing this application.

As used herein, “Hyperpolarization Activated Cyclic Nucleotide Gated Potassium Channel 4,” used interchangeably with the term “HCN4,” refers to a member of the hyperpolarization-activated cyclic nucleotide-gated (HCN) channel family. The HCN4 channel transports positively charged ions into heart muscle cells. This channel is located primarily in the sino-atrial (SA) node, which is an area of specialized cells in the heart that functions as a natural pacemaker. The HCN4 channel allows potassium and sodium ions to flow into cells of the SA node. This ion flow is often called the “pacemaker current” because it generates electrical impulses that start each heartbeat and is involved in maintaining a regular heart rhythm. HCN4 is also known as Potassium/Sodium Hyperpolarization-Activated Cyclic Nucleotide-Gated Channel 4, Hyperpolarization Activated Cyclic Nucleotide-Gated Potassium Channel 4, Hyperpolarization Activated Cyclic Nucleotide-Gated Cation Channel 4 or SSS2.

An exemplary sequence of a human HCN4 mRNA transcript can be found at, for example, GenBank Accession No. GI: 1519312820 (NM_005477.3; SEQ ID NO: 61; reverse complement, SEQ ID NO: 62). The sequence of mouse HCN4 mRNA can be found at, for example, GenBank Accession No. GI: 1686254400 (NM_001081192.3; SEQ ID NO: 63; reverse complement, SEQ ID NO: 64). The sequence of rat HCN4 mRNA can be found at, for example, GenBank Accession No. GI: 1937893976 (NM 021658.2; SEQ ID NO: 65; reverse complement, SEQ ID NO: 66). The sequence of Macaca mulatta HCN4 mRNA can be found at, for example, GenBank Accession No. GI: 1622953870 (XM_002804859.3; SEQ ID NO: 67; reverse complement, SEQ ID NO: 68). The sequence of Macaca fascicularis HCN4 mRNA can be found at, for example, GenBank Accession No. GI: 982258526 (XM_005559993.2; SEQ ID NO: 69; reverse complement, SEQ ID NO: 70).

Additional examples of HCN4 mRNA sequences are readily available through publicly available databases, e.g., GenBank, UniProt, OMIM, UCSC Genome Browser, and the Macaca genome project web site.

Further information on HCN4 can be found, for example, at www.ncbi.nlm.nih.gov/gene/?term=HCN4.

The entire contents of each of the foregoing GenBank Accession numbers and the Gene database numbers are incorporated herein by reference as of the date of filing this application.

The term HCN4, as used herein, also refers to variations of the HCN4 gene including variants provided in the SNP database. Numerous sequence variations within the HCN4 gene have been identified and may be found at, for example, NCBI dbSNP and UniProt (see, e.g., www.ncbi.nlm.nih.gov/gene/?term=HCN4, the entire contents of which is incorporated herein by reference as of the date of filing this application.

As used herein, “Hyperpolarization Activated Cyclic Nucleotide Gated Potassium Channel 3,” used interchangeably with the term “HCN3,” refers to a member of the hyperpolarization-activated cyclic nucleotide-gated (HCN) channel family. A study conducted in the mouse suggested that HCN3 channels might be involved in the regulation of the circadian system. HCN3 channels have also been reported to be present in the intergeniculate leaflet of the hypothalamus. HCN3 is also known as Potassium/Sodium Hyperpolarization-Activated Cyclic Nucleotide-Gated Channel 3, Hyperpolarization Activated Cyclic Nucleotide-Gated Potassium Channel 3, or KIAA1535.

An exemplary sequence of a human HCN3 mRNA transcript can be found at, for example, GenBank Accession No. GI: 1519312303 (NM_020897.3; SEQ ID NO: 71; reverse complement, SEQ ID NO: 72). The sequence of mouse HCN3 mRNA can be found at, for example, GenBank Accession No. GI: 6680190 (NM_008227.1; SEQ ID NO: 73; reverse complement, SEQ ID NO: 74). The sequence of rat HCN3 mRNA can be found at, for example, GenBank Accession No. GI: 16758501 (NM_053685.1; SEQ ID NO: 75; reverse complement, SEQ ID NO: 76). The sequence of Macaca mulatta HCN3 mRNA can be found at, for example, GenBank Accession No. GI: 1622829938 (XM_001115891.4; SEQ ID NO: 77; reverse complement, SEQ ID NO: 78). The sequence of Macaca fascicularis HCN3 mRNA can be found at, for example, GenBank Accession No. GI: 982225310 (XM_005541549.2; SEQ ID NO: 79; reverse complement, SEQ ID NO: 80).

Additional examples of HCN3 mRNA sequences are readily available through publicly available databases, e.g., GenBank, UniProt, OMIM, UCSC Genome Browser, and the Macaca genome project web site.

Further information on HCN3 can be found, for example, at www.ncbi.nlm.nih.gov/gene/?term=HCN3.

The entire contents of each of the foregoing GenBank Accession numbers and the Gene database numbers are incorporated herein by reference as of the date of filing this application.

The term HCN3, as used herein, also refers to variations of the HCN3 gene including variants provided in the SNP database. Numerous sequence variations within the HCN3 gene have been identified and may be found at, for example, NCBI dbSNP and UniProt (see, e.g., www.ncbi.nlm.nih.gov/gene/?term=HCN3, the entire contents of which is incorporated herein by reference as of the date of filing this application.

As used herein, “Potassium Voltage-Gated Channel Subfamily A Member 5,” used interchangeably with the term “KCNA5,” refers to a member of the voltage-gated potassium channel family. The Voltage-gated potassium channels mediate transmembrane potassium transport in excitable membranes. These channels form tetrameric potassium-selective channels through which potassium ions pass in accordance with their electrochemical gradient, and alternate between opened and closed conformations in response to the voltage difference across the membrane. KCNA5 contains six membrane-spanning domains with a shaker-type repeat in the fourth segment. It belongs to the delayed rectifier class, the function of which could restore the resting membrane potential of beta cells after depolarization and thereby contribute to the regulation of insulin secretion. KCNA5 is also known as HPCN1, HK2, Potassium Voltage-Gated Channel, Shaker-Related Subfamily, Member 5; Voltage-Gated Potassium Channel Subunit Kv1.5; Voltage-Gated Potassium Channel HK2; Kv1.5; Insulinoma And Islet Potassium Channel; Cardiac Potassium Channel; Potassium Channel 1; ATFB7, HCK1 or PCN1.

An exemplary sequence of a human KCNA5 mRNA transcript can be found at, for example, GenBank Accession No. GI: 1653961222 (NM_002234.4; SEQ ID NO: 81; reverse complement, SEQ ID NO: 82). The sequence of mouse KCNA5 mRNA can be found at, for example, GenBank Accession No. GI: 158937280 (NM_145983.2; SEQ ID NO: 83; reverse complement, SEQ ID NO: 84). The sequence of rat KCNA5 mRNA can be found at, for example, GenBank Accession No. GI: 6981117 (NM_012972.1; SEQ ID NO: 85; reverse complement, SEQ ID NO: 86). The sequence of Macaca mulatta KCNA5 mRNA can be found at, for example, GenBank Accession No. GI: 1622843572 (XM_001102294.4; SEQ ID NO: 87; reverse complement, SEQ ID NO: 88). The sequence of Macaca fascicularis KCNA5 mRNA can be found at, for example, GenBank Accession No. GI: 982279162 (XM_005569870.2; SEQ ID NO: 89; reverse complement, SEQ ID NO: 90).

Additional examples of KCNA5 mRNA sequences are readily available through publicly available databases, e.g., GenBank, UniProt, OMIM, UCSC Genome Browser, and the Macaca genome project web site.

Further information on KCNA5 can be found, for example, at www.ncbi.nlm.nih.gov/gene/?term=KCNA5.

The entire contents of each of the foregoing GenBank Accession numbers and the Gene database numbers are incorporated herein by reference as of the date of filing this application.

The term KCNA5, as used herein, also refers to variations of the KCNA5 gene including variants provided in the SNP database. Numerous sequence variations within the KCNA5 gene have been identified and may be found at, for example, NCBI dbSNP and UniProt (see, e.g., www.ncbi.nlm.nih.gov/gene/?term=KCNA5, the entire contents of which is incorporated herein by reference as of the date of filing this application.

As used herein, “Potassium Inwardly Rectifying Channel Subfamily J Member 3,” used interchangeably with the term “KCNJ3,” refers to an integral membrane protein and an inward-rectifier type potassium channel. The inward-rectifier type potassium channels have a greater tendency to allow potassium to flow into a cell rather than out of a cell. This asymmetry in potassium ion conductance plays a key role in the excitability of muscle cells and neurons. KCNJ3 is controlled by G-proteins and plays an important role in regulating heartbeat. It associates with three other G-protein-activated potassium channels to form a heteromultimeric pore-forming complex, which also couples to neurotransmitter receptors in the brain. These multimeric G-protein-gated inwardly-rectifying potassium (GIRK) channels have a wide range of physiological roles, including the regulation of heartbeat, reward mechanisms, learning and memory functions, blood platelet aggregation, insulin secretion, and lipid metabolism. KCNJ3 is also known as GIRK1, G Protein-Activated Inward Rectifier Potassium Channel 1, KGA; Potassium Channel, Inwardly Rectifying Subfamily J Member 3; Inward Rectifier K(+) Channel Kir3.1; or Potassium Inwardly-Rectifying Channel Subfamily J Member 3 Splice Variant 1e.

An exemplary sequence of a human KCNJ3 mRNA transcript can be found at, for example, GenBank Accession No. GI: 1519246021 (NM_002239.4; SEQ ID NO: 91; reverse complement, SEQ ID NO: 92). The sequence of mouse KCNJ3 mRNA can be found at, for example, GenBank Accession No. GI: 756398330 (NM_008426.2; SEQ ID NO: 93; reverse complement, SEQ ID NO: 94). The sequence of rat KCNJ3 mRNA can be found at, for example, GenBank Accession No. GI: 148747456 (NM_031610.3; SEQ ID NO: 95; reverse complement, SEQ ID NO: 96). The sequence of Macaca mulatta KCNJ3 mRNA can be found at, for example, GenBank Accession No. GI: 387849010 (NM_001261696.1; SEQ ID NO: 97; reverse complement, SEQ ID NO: 98). The sequence of Macaca fascicularis KCNJ3 mRNA can be found at, for example, GenBank Accession No. GI: 982285759 (XM_005573205.2; SEQ ID NO: 99; reverse complement, SEQ ID NO: 100).

Additional examples of KCNJ3 mRNA sequences are readily available through publicly available databases, e.g., GenBank, UniProt, OMIM, UCSC Genome Browser, and the Macaca genome project web site.

Further information on KCNJ3 can be found, for example, at www.ncbi.nlm.nih.gov/gene/?term=KCNJ3.

The entire contents of each of the foregoing GenBank Accession numbers and the Gene database numbers are incorporated herein by reference as of the date of filing this application.

The term KCNJ3, as used herein, also refers to variations of the KCNJ3 gene including variants provided in the SNP database. Numerous sequence variations within the KCNJ3 gene have been identified and may be found at, for example, NCBI dbSNP and UniProt (see, e.g., www.ncbi.nlm.nih.gov/gene/?term=KCNJ3, the entire contents of which is incorporated herein by reference as of the date of filing this application.

As used herein, “Potassium Inwardly Rectifying Channel Subfamily J Member 4,” used interchangeably with the term “KCNJ4,” refers to an integral membrane protein and inward-rectifier type potassium channel. The inward-rectifier type potassium channels have a greater tendency to allow potassium to flow into a cell rather than out of a cell. This asymmetry in potassium ion conductance plays a key role in the excitability of muscle cells and neurons. KCNJ4 can tetramerize to form functional inwardly rectifying channels, in which each monomer contains two transmembrane helix domains, an ion-selective P-loop, and cytoplasmic N- and C-terminal domains. The distribution of KCNJ4 is predominantly focused in both heart and brain, especially in the cardiac myocytes and forebrain region. KCNJ4 may play important roles in the regulation of resting membrane potential, cellular excitability and potassium homeostasis in the nervous system and various peripheral tissues. KCNJ4 is also known as HIRK2, HRK1, IRK3, HIR, Kir2.3, inward rectifier potassium channel 4; Inward Rectifier K(+) Channel Kir2.3; Potassium Voltage-Gated Channel Subfamily J Member 4; Hippocampal Inward Rectifier Potassium Channel; or Hippocampal Inward Rectifier.

An exemplary sequence of a human KCNJ4 mRNA transcript can be found at, for example, GenBank Accession No. GI: 1732746379 (NM_152868.3; SEQ ID NO: 101; reverse complement, SEQ ID NO: 102). The sequence of mouse KCNJ4 mRNA can be found at, for example, GenBank Accession No. GI: 1720383422 (XM_006520486.4; SEQ ID NO: 103; reverse complement, SEQ ID NO: 104). The sequence of rat KCNJ4 mRNA can be found at, for example, GenBank Accession No. GI: 1937901561 (NM 053870.3; SEQ ID NO: 105; reverse complement, SEQ ID NO: 106). The sequence of Macaca mulatta KCNJ4 mRNA can be found at, for example, GenBank Accession No. GI: 1622838042 (XM_015150354.2; SEQ ID NO: 107; reverse complement, SEQ ID NO: 108). The sequence of Macaca fascicularis KCNJ4 mRNA can be found at, for example, GenBank Accession No. GI: 544461851 (XM_005567299.1; SEQ ID NO: 109; reverse complement, SEQ ID NO: 110).

Additional examples of KCNJ4 mRNA sequences are readily available through publicly available databases, e.g., GenBank, UniProt, OMIM, UCSC Genome Browser, and the Macaca genome project web site.

Further information on KCNJ4 can be found, for example, at www.ncbi.nlm.nih.gov/gene/?term=KCNJ4.

The entire contents of each of the foregoing GenBank Accession numbers and the Gene database numbers are incorporated herein by reference as of the date of filing this application.

The term KCNJ4, as used herein, also refers to variations of the KCNJ4 gene including variants provided in the SNP database. Numerous sequence variations within the KCNJ4 gene have been identified and may be found at, for example, NCBI dbSNP and UniProt (see, e.g., www.ncbi.nlm.nih.gov/gene/?term=KCNJ4, the entire contents of which is incorporated herein by reference as of the date of filing this application. As used herein, “Phosphodiesterase 1,” used interchangeably with the term “PDE1,” refers to a member of the cyclic nucleotide phosphodiesterases families. Cyclic nucleotide phosphodiesterases (PDEs) are superfamily of enzymes that regulate the spatial and temporal relationship of second messenger signaling in the cellular system. Among the 11 different families of PDEs, phosphodiesterase 1 (PDE1) sub-family of enzymes hydrolyze both 3′,5′-cyclic adenosine monophosphate (cAMP) and 3′,5′-cyclic guanosine monophosphate (cGMP) in a mutually competitive manner. The catalytic activity of PDE1 is stimulated by their binding to Ca2+/calmodulin (CaM), resulting in the integration of Ca2+ and cyclic nucleotide-mediated signaling in various diseases. The PDE1 family includes three subtypes, PDE1A, PDE1B and PDE1C, which differ for their relative affinities for cAMP and cGMP. These isoforms are differentially expressed throughout the body, including the cardiovascular, central nervous system and other organs. Thus, PDE1 enzymes play a critical role in the pathophysiology of diseases through the fundamental regulation of cAMP and cGMP signaling. PDE1 is also known as Calcium/Calmodulin-Dependent 3′,5′-Cyclic Nucleotide Phosphodiesterase 1; Calcium/Calmodulin-Stimulated Cyclic Nucleotide Phosphodiesterase; CAM-PDE 1, HSPDE1, HCAM1, or EC 3.1.4.

An exemplary sequence of a human PDE1 mRNA transcript can be found at, for example, GenBank Accession No. GI: 2062580163 (NM_005019.7; SEQ ID NO: 111; reverse complement, SEQ ID NO: 112). The sequence of mouse PDE1 mRNA can be found at, for example, GenBank Accession No. GI: 227330628 (NM_001159582.1; SEQ ID NO: 113; reverse complement, SEQ ID NO: 114). The sequence of rat PDE1 mRNA can be found at, for example, GenBank Accession No. GI: 13540702 (NM_030871.1; SEQ ID NO: 115; reverse complement, SEQ ID NO: 116). The sequence of Macaca mulatta PDE1 mRNA can be found at, for example, GenBank Accession No. GI: 383872283 (NM_001257584.1; SEQ ID NO: 117; reverse complement, SEQ ID NO: 118). The sequence of Macaca fascicularis PDE1 mRNA can be found at, for example, GenBank Accession No. GI: 982286500 (XR_001483985.1; SEQ ID NO: 119; reverse complement, SEQ ID NO: 120).

Additional examples of PDE1 mRNA sequences are readily available through publicly available databases, e.g., GenBank, UniProt, OMIM, UCSC Genome Browser, and the Macaca genome project web site.

Further information on PDE1 can be found, for example, at www.ncbi.nlm.nih.gov/gene/?term=PDE1.

The entire contents of each of the foregoing GenBank Accession numbers and the Gene database numbers are incorporated herein by reference as of the date of filing this application.

The term PDE1, as used herein, also refers to variations of the PDE1 gene including variants provided in the SNP database. Numerous sequence variations within the PDE1 gene have been identified and may be found at, for example, NCBI dbSNP and UniProt (see, e.g., www.ncbi.nlm.nih.gov/gene/?term=PDE1, the entire contents of which is incorporated herein by reference as of the date of filing this application.

As used herein, “Delta 4-Desaturase, Sphingolipid 1,” used interchangeably with the term “DEGS1,” refers to a member of the membrane fatty acid desaturase family which is responsible for inserting double bonds into specific positions in fatty acids. DEGS1 is an enzyme that catalyzes the final step in the ceramide biosynthesis pathway. Ceramides have emerged as important regulators of tissue metabolism that play essential roles in cardiometabolic disease. They are potent biomarkers of diabetes and heart disease and are now being measured clinically as predictors of major adverse cardiac events. Moreover, studies in rodents reveal that inhibitors of ceramide synthesis prevent or reverse the pathogenic features of type 2 diabetes, nonalcoholic fatty liver disease, atherosclerosis, and cardiomyopathy. Therefore, inhibition of DEGS1 is considered as a potential therapeutic approach to lower ceramides and combat cardiometabolic disease.

DEGS1 is also known as MLD, DES-1, FADS7, Cell Migration-Inducing Gene 15 Protein, Sphingolipid Delta(4)-Desaturase DES1, Dihydroceramide Desaturase 1, Membrane Lipid Desaturase, Degenerative Spermatocyte Homolog 1, Lipid Desaturase, Membrane Fatty Acid (Lipid) Desaturase, Migration-Inducing Gene 15 Protein, Sphingolipid Delta 4 Desaturase, EC 1.14.19.17, HLD18, MIG15 and DEGS.

An exemplary sequence of a human DEGS1 mRNA transcript can be found at, for example, GenBank Accession No. GI: 1519243257 (NM_003676.4; SEQ ID NO:121; reverse complement, SEQ ID NO: 122). The sequence of mouse DEGS1 mRNA can be found at, for example, GenBank Accession No. GI: 1343071492 (NM_007853.5; SEQ ID NO: 123; reverse complement, SEQ ID NO: 124). The sequence of rat DEGS1 mRNA can be found at, for example, GenBank Accession No. GI: 162287183 (NM_053323.2; SEQ ID NO: 125; reverse complement, SEQ ID NO: 126). The sequence of Macaca fascicularis DEGS1 mRNA can be found at, for example, GenBank Accession No. GI: 982223631 (XM_005540946.2; SEQ ID NO: 127; reverse complement, SEQ ID NO: 128). The sequence of Macaca mulatta DEGS1 mRNA can be found at, for example, GenBank Accession No. GI: 388452769 (NM_001266006.1; SEQ ID NO: 129; reverse complement, SEQ ID NO: 130).

Additional examples of DEGS1 mRNA sequences are readily available through publicly available databases, e.g., GenBank, UniProt, OMIM, UCSC Genome Browser, and the Macaca genome project web site. Further information on DEGS1 can be found, for example, at www.ncbi.nlm.nih.gov/gene/?term=DEGS1.

The entire contents of each of the foregoing GenBank Accession numbers and the Gene database numbers are incorporated herein by reference as of the date of filing this application.

The term DEGS1, as used herein, also refers to variations of the DEGS1 gene including variants provided in the SNP database. Numerous sequence variations within the DEGS1 gene have been identified and may be found at, for example, NCBI dbSNP and UniProt (see, e.g., www.ncbi.nlm.nih.gov/gene/?term=DEGS1, the entire contents of which is incorporated herein by reference as of the date of filing this application.

As used herein, “leptin,” used interchangeably with the term “LEP,” refers to a protein that is secreted by white adipocytes into the circulation and plays a major role in the regulation of energy homeostasis. Circulating leptin binds to the leptin receptor in the brain, which activates downstream signaling pathways that inhibit feeding and promote energy expenditure. This protein also has several endocrine functions, and is involved in the regulation of immune and inflammatory responses, hematopoiesis, angiogenesis, reproduction, bone formation and wound healing. Mutations in this gene and its regulatory regions cause severe obesity and morbid obesity with hypogonadism in human patients. A mutation in this gene has also been linked to type 2 diabetes mellitus development. Leptin is also known as OBS, OB, obese, obesity factor, or LEPD.

An exemplary sequence of a human leptin mRNA transcript can be found at, for example, GenBank Accession No. GI: 1519312816 (NM_000230.3; SEQ ID NO:131; reverse complement, SEQ ID NO: 132). The sequence of mouse leptin mRNA can be found at, for example, GenBank Accession No. GI: 34328437 (NM_008493.3; SEQ ID NO:133; reverse complement, SEQ ID NO: 134). The sequence of rat leptin mRNA can be found at, for example, GenBank Accession No. GI: 291463266 (NM_013076.3; SEQ ID NO: 135; reverse complement, SEQ ID NO: 136). The sequence of Macaca fascicularis leptin mRNA can be found at, for example, GenBank Accession No. GI: 982241369 (XM_005550685.2; SEQ ID NO: 137; reverse complement, SEQ ID NO: 138). The sequence of Macaca mulatta leptin mRNA can be found at, for example, GenBank Accession No. GI: 112363108 (NM_001042755.1; SEQ ID NO: 139; reverse complement, SEQ ID NO: 140).

Additional examples of leptin mRNA sequences are readily available through publicly available databases, e.g., GenBank, UniProt, OMIM, UCSC Genome Browser, and the Macaca genome project web site. Further information on leptin can be found, for example, at www.ncbi.nlm.nih.gov/gene/?term=leptin.

The entire contents of each of the foregoing GenBank Accession numbers and the Gene database numbers are incorporated herein by reference as of the date of filing this application.

The term leptin, as used herein, also refers to variations of the leptin gene including variants provided in the SNP database. Numerous sequence variations within the leptin gene have been identified and may be found at, for example, NCBI dbSNP and UniProt (see, e.g., www.ncbi.nlm.nih.gov/gene/?term=leptin, the entire contents of which is incorporated herein by reference as of the date of filing this application.

As used herein, “folliculin,” used interchangeably with the term “FLCN,” refers to a protein that is related to Birt-Hogg-Dubé syndrome, primary spontaneous pneumothorax and some types of nonhereditary (sporadic) tumors. The folliculin protein is present in many of the body's tissues, including the brain, heart, placenta, testis, skin, lung, and kidney. Folliculin may be important for cells' uptake of foreign particles (endocytosis or phagocytosis). The protein may also play a role in the structural framework that helps to define the shape, size, and movement of a cell (the cytoskeleton) and in interactions between cells. FLCN is also known as BHD, DENND8B, BHD Skin Lesion Fibrofolliculoma Protein, Birt-Hogg-Dube Syndrome Protein, MGC17998, MGC23445 or FLCL.

An exemplary sequence of a human FLCN mRNA transcript can be found at, for example, GenBank Accession No. GI: 1519312711 (NM_144997.7; SEQ ID NO:141; reverse complement, SEQ ID NO: 142). The sequence of mouse FLCN mRNA can be found at, for example, GenBank Accession No. GI: 405778334 (NM_001271356.1; SEQ ID NO: 143; reverse complement, SEQ ID NO: 144). The sequence of rat FLCN mRNA can be found at, for example, GenBank Accession No. GI: 55742811 (NM_199390.2; SEQ ID NO: 145; reverse complement, SEQ ID NO: 146). The sequence of Macaca fascicularis FLCN mRNA can be found at, for example, GenBank Accession No. GI: 982303338 (XM_005583008.2; SEQ ID NO: 147; reverse complement, SEQ ID NO: 148). The sequence of Macaca mulatta FLCN mRNA can be found at, for example, GenBank Accession No. GI: 388490399 (NM_001266691.1; SEQ ID NO: 149; reverse complement, SEQ ID NO: 150).

Additional examples of FLCN mRNA sequences are readily available through publicly available databases, e.g., GenBank, UniProt, OMIM, UCSC Genome Browser, and the Macaca genome project web site. Further information on FLCN can be found, for example, at www.ncbi.nlm.nih.gov/gene/?term=FLCN.

The entire contents of each of the foregoing GenBank Accession numbers and the Gene database numbers are incorporated herein by reference as of the date of filing this application.

The term FLCN, as used herein, also refers to variations of the FLCN gene including variants provided in the SNP database. Numerous sequence variations within the FLCN gene have been identified and may be found at, for example, NCBI dbSNP and UniProt (see, e.g., www.ncbi.nlm.nih.gov/snp/?term=FLCN, the entire contents of which is incorporated herein by reference as of the date of filing this application.

As used herein, “Zinc Finger Protein 423,” used interchangeably with the term “ZFP423,” refers to a nuclear protein that belongs to the family of Kruppel-like C2H2 zinc finger proteins. It functions as a DNA-binding transcription factor by using distinct zinc fingers in different signaling pathways. Thus, it is thought that this gene may have multiple roles in signal transduction during development. Mutations in this gene are associated with nephronophthisis-14 and Joubert syndrome-19.

ZFP423 is also known as NPHP14, HOAZ, OAZ, KIAA0760, Zfp104, JBTS19, Ebfaz, Early B-Cell Factor Associated Zinc Finger Protein, Smad- And Olf-Interacting Zinc Finger Protein, Olf1/EBF-Associated Zinc Finger Protein, or Roaz.

An exemplary sequence of a human ZFP423 mRNA transcript can be found at, for example, GenBank Accession No. GI: 1889411210 (NM_015069.5; SEQ ID NO:151; reverse complement, SEQ ID NO: 152). The sequence of mouse ZFP423 mRNA can be found at, for example, GenBank Accession No. GI: 46359076 (NM_033327.2; SEQ ID NO:153; reverse complement, SEQ ID NO: 154). The sequence of rat ZFP423 mRNA can be found at, for example, GenBank Accession No. GI: 1997589018 (NM_001393718.1; SEQ ID NO:155; reverse complement, SEQ ID NO: 156). The sequence of Macaca fascicularis ZFP423 mRNA can be found at, for example, XM_005591872.2; (SEQ ID NO: 157; reverse complement, SEQ ID NO: 158). The sequence of Macaca mulatta ZFP423 mRNA can be found at, for example, XM_015126090.2; SEQ ID NO: 159; reverse complement, SEQ ID NO: 160).

Additional examples of ZFP423 mRNA sequences are readily available through publicly available databases, e.g., GenBank, UniProt, OMIM, UCSC Genome Browser, and the Macaca genome project web site. Further information on ZFP423 can be found, for example, at www.ncbi.nlm.nih.gov/gene/?term=ZFP423.

The entire contents of each of the foregoing GenBank Accession numbers and the Gene database numbers are incorporated herein by reference as of the date of filing this application.

The term ZFP423, as used herein, also refers to variations of the ZFP423 gene including variants provided in the SNP database. Numerous sequence variations within the ZFP423 gene have been identified and may be found at, for example, NCBI dbSNP and UniProt (see, e.g., www.ncbi.nlm.nih.gov/snp/?term=ZFP423, the entire contents of which is incorporated herein by reference as of the date of filing this application.

As used herein, “Cyclin Dependent Kinase 6,” used interchangeably with the term “CDK6,” refers to a member of the CMGC family of serine/threonine protein kinases. This kinase is a catalytic subunit of the protein kinase complex that is important for cell cycle G1 phase progression and G1/S transition. The activity of this kinase first appears in mid-G1 phase, which is controlled by the regulatory subunits including D-type cyclins and members of INK4 family of CDK inhibitors. This kinase, as well as CDK4, has been shown to phosphorylate, and thus regulate the activity of, tumor suppressor protein Rb. Altered expression of this gene has been observed in multiple human cancers. A mutation in this gene resulting in reduced cell proliferation, and impaired cell motility and polarity, and has been identified in patients with primary microcephaly. CDK6 is also known as PLSTIRE, Serine/Threonine-Protein Kinase PLSTIRE, Cell Division Protein Kinase 6, EC 2.7.11.22, MCPH12 or CDKN6.

An exemplary sequence of a human CDK6 mRNA transcript can be found at, for example, GenBank Accession No. GI: 1677500223 (NM_001259.8; SEQ ID NO: 161; reverse complement, SEQ ID NO: 162). The sequence of mouse CDK6 mRNA can be found at, for example, GenBank Accession No. GI: 922304379 (NM_009873.3; SEQ ID NO: 163; reverse complement, SEQ ID NO: 164). The sequence of rat CDK6 mRNA can be found at, for example, GenBank Accession No. GI: 1982560006 (NM_001191861.2; SEQ ID NO:165; reverse complement, SEQ ID NO: 166). The sequence of Macaca fascicularis CDK6 mRNA can be found at, for example, GenBank Accession No. GI: 982240553 (XM_015447745.1; SEQ ID NO: 167; reverse complement, SEQ ID NO: 168). The sequence of Macaca mulatta CDK6 mRNA can be found at, for example, GenBank Accession No. GI: 386782158 (NM_001261307.1; SEQ ID NO: 169; reverse complement, SEQ ID NO: 170).

Additional examples of CDK6 mRNA sequences are readily available through publicly available databases, e.g., GenBank, UniProt, OMIM, UCSC Genome Browser, and the Macaca genome project web site. Further information on CDK6 can be found, for example, at www.ncbi.nlm.nih.gov/gene/?term=CDK6.

The entire contents of each of the foregoing GenBank Accession numbers and the Gene database numbers are incorporated herein by reference as of the date of filing this application.

The term CDK6, as used herein, also refers to variations of the CDK6 gene including variants provided in the SNP database. Numerous sequence variations within the CDK6 gene have been identified and may be found at, for example, NCBI dbSNP and UniProt (see, e.g., www.ncbi.nlm.nih.gov/gene/?term=CDK6, the entire contents of which is incorporated herein by reference as of the date of filing this application.

As used herein, “Regulatory Associated Protein Of MTOR Complex 1,” used interchangeably with the term “RPTOR,” refers to a component of a signaling pathway that regulates cell growth in response to nutrient and insulin levels. The encoded protein forms a stoichiometric complex with the mTOR kinase, and also associates with eukaryotic initiation factor 4E-binding protein-1 and ribosomal protein S6 kinase. The protein positively regulates the downstream effector ribosomal protein S6 kinase, and negatively regulates the mTOR kinase. Mutations of RPTOR have been observed in cancers such as intestinal cancer, skin cancer, and stomach cancer. RPTOR is also known as Raptor, KIAA1303, KOG1, Mip1, Regulatory-Associated Protein Of MTOR, or P150 Target Of Rapamycin (TOR)-Scaffold Protein Containing WD-Repeats.

An exemplary sequence of a human RPTOR mRNA transcript can be found at, for example, GenBank Accession No. GI: 1519244773 (NM_020761.3; SEQ ID NO:171; reverse complement, SEQ ID NO: 172). The sequence of mouse RPTOR mRNA can be found at, for example, GenBank Accession No. GI: 807045913 (NM_028898.3; SEQ ID NO: 173; reverse complement, SEQ ID NO: 174). The sequence of rat RPTOR mRNA can be found at, for example, GenBank Accession No. GI: 260166602 (NM_001134499.2; SEQ ID NO: 175; reverse complement, SEQ ID NO: 176). The sequence of Macaca fascicularis RPTOR mRNA can be found at, for example, GenBank Accession No. GI: 982307196 (XM_005585210.2; SEQ ID NO: 177; reverse complement, SEQ ID NO: 178). The sequence of Macaca mulatta RPTOR mRNA can be found at, for example, GenBank Accession No. GI: 1622881944 (XM_015120520.2; SEQ ID NO: 179; reverse complement, SEQ ID NO: 180).

Additional examples of RPTOR mRNA sequences are readily available through publicly available databases, e.g., GenBank, UniProt, OMIM, UCSC Genome Browser, and the Macaca genome project web site. Further information on RPTOR can be found, for example, at www.ncbi.nlm.nih.gov/gene/?term=RPTOR.

The entire contents of each of the foregoing GenBank Accession numbers and the Gene database numbers are incorporated herein by reference as of the date of filing this application.

The term RPTOR, as used herein, also refers to variations of the RPTOR gene including variants provided in the SNP database. Numerous sequence variations within the RPTOR gene have been identified and may be found at, for example, NCBI dbSNP and UniProt (see, e.g., www.ncbi.nlm.nih.gov/snp/?term=RPTOR, the entire contents of which is incorporated herein by reference as of the date of filing this application.

As used herein, “Mechanistic Target Of Rapamycin Kinase,” used interchangeably with the term “mTOR,” refers to an atypical serine/threonine kinase of 289 kDa that belongs to the family of the phosphoinositide 3-kinase related kinase. These kinases mediate cellular responses to stresses such as DNA damage and nutrient deprivation. Specifically, mTOR is the intracellular kinase linking nutrient availability with metabolic control, and its deregulation is a hallmark of diabetes and cancer. The mTOR kinase is encoded by a single gene in mammals, but it exerts its main cellular functions by forming mTORC1 and mTORC2 through assembly with specific adaptor proteins. mTORC1 controls protein synthesis, cell growth and proliferation, and mTORC2 is a regulator of the actin cytoskeleton, and promotes cell survival and cell cycle progression. mTOR is also known as RAFT1, Rapamycin And FKBP12 Target 1, Mammalian Target Of Rapamycin, FRAP1, FRAP2, FRAP, FK506-Binding Protein 12-Rapamycin Complex-Associated Protein 1, Serine/Threonine-Protein Kinase MTOR, Rapamycin Associated Protein FRAP2, FLJ44809, DJ576K7.1, FK506 Binding Protein 12-Rapamycin Associated Protein 1, FKBP12-Rapamycin Complex-Associated Protein, Rapamycin Target Protein, EC 2.7.11.1, or SKS.

An exemplary sequence of a human mTOR mRNA transcript can be found at, for example, GenBank Accession No. GI: 1653961062 (NM_004958.4; SEQ ID NO:181; reverse complement, SEQ ID NO: 182). The sequence of mouse mTOR mRNA can be found at, for example, GenBank Accession No. GI: 227330585 (NM_020009.2; SEQ ID NO: 183; reverse complement, SEQ ID NO: 184). The sequence of rat mTOR mRNA can be found at, for example, GenBank Accession No. GI: 1935257123 (NM_019906.2; SEQ ID NO:185; reverse complement, SEQ ID NO: 186). The sequence of Macaca fascicularis mTOR mRNA can be found at, for example, GenBank Accession No. GI: 982230273 (XM_005544805.2; SEQ ID NO: 187; reverse complement, SEQ ID NO: 188). The sequence of Macaca mulatta mTOR mRNA can be found at, for example, GenBank Accession No. GI: 1622834993 (XM_015111100.2; SEQ ID NO: 189; reverse complement, SEQ ID NO: 190).

Additional examples of mTOR mRNA sequences are readily available through publicly available databases, e.g., GenBank, UniProt, OMIM, UCSC Genome Browser, and the Macaca genome project web site. Further information on mTOR can be found, for example, at www.ncbi.nlm.nih.gov/gene/?term=mTOR.

The entire contents of each of the foregoing GenBank Accession numbers and the Gene database numbers are incorporated herein by reference as of the date of filing this application.

The term mTOR, as used herein, also refers to variations of the mTOR gene including variants provided in the SNP database. Numerous sequence variations within the mTOR gene have been identified and may be found at, for example, NCBI dbSNP and UniProt (see, e.g., www.ncbi.nlm.nih.gov/smp/?term=mTOR the entire contents of which is incorporated herein by reference as of the date of filing this application.

As used herein, “Forkhead Box P1,” used interchangeably with the term “FOXP1,” refers to a member of the subfamily P of the forkhead box (FOX) transcription factor family. Forkhead box transcription factors play important roles in the regulation of tissue- and cell type-specific gene transcription during both development and adulthood. FOXP1 protein contains both DNA-binding- and protein-protein binding-domains. Previous studies have investigated the biological roles of the transcription factor FOXP1 in brown/beige adipocyte differentiation and thermogenesis. Adipose-specific deletion of FOXP1 leads to an increase of brown adipose activity and browning program of white adipose tissues. The FOXP1-deficient mice show an augmented energy expenditure and are protected from diet-induced obesity and insulin resistance. Consistently, overexpression of FOXP1 in adipocytes impairs adaptive thermogenesis and promotes diet-induced obesity. Thus, FOXP1 provides an important clue for its targeting and treatment of obesity. FOXP1 is also known as HSPC215, HFKH1B, 12CC4, QRF1, Fork Head-Related Protein Like B, Mac-1-Regulated Forkhead, Glutamine-Rich Factor 1, MFH or PAX5/FOXP1 Fusion Protein.

An exemplary sequence of a human FOXP1 mRNA transcript can be found at, for example, GenBank Accession No. GI: 1777535708 (NM_032682.6; SEQ ID NO:191; reverse complement, SEQ ID NO: 192). The sequence of mouse FOXP1 mRNA can be found at, for example, GenBank Accession No. GI: 309319789 (NM_053202.2; SEQ ID NO: 193; reverse complement, SEQ ID NO: 194). The sequence of rat FOXP1 mRNA can be found at, for example, GenBank Accession No. GI: 1937889958 (NM_001034131.2 SEQ ID NO:195; reverse complement, SEQ ID NO: 196). The sequence of Macaca fascicularis FOXP1 mRNA can be found at, for example, GenBank Accession No. GI: 982232930 (XM_005547604.2; SEQ ID NO: 197; reverse complement, SEQ ID NO: 198). The sequence of Macaca mulatta FOXP1 mRNA can be found at, for example, GenBank Accession No. GI: 388453320 (NM_001266321.1; SEQ ID NO: 199; reverse complement, SEQ ID NO: 200).

Additional examples of FOXP1 mRNA sequences are readily available through publicly available databases, e.g., GenBank, UniProt, OMIM, UCSC Genome Browser, and the Macaca genome project web site. Further information on FOXP1 can be found, for example, at www.ncbi.nlm.nih.gov/gene/?term=FOXP1.

The entire contents of each of the foregoing GenBank Accession numbers and the Gene database numbers are incorporated herein by reference as of the date of filing this application.

The term FOXP1, as used herein, also refers to variations of the FOXP1gene including variants provided in the SNP database. Numerous sequence variations within the FOXP1 gene have been identified and may be found at, for example, NCBI dbSNP and UniProt (see, e.g., www.ncbi.nlm.nih.gov/gene/?term=FOXP1, the entire contents of which is incorporated herein by reference as of the date of filing this application.

As used herein, “Phosphodiesterase 3B,” used interchangeably with the term “PDE3B,” refers to an isoform of the PDE3 family of cyclic nucleotide phosphodiesterases. Cyclic nucleotide phosphodiesterases regulate intracellular signalling by hydrolysing cAMP and/or cGMP. Enzymes in the PDE3 family of phosphodiesterases are dual-specificity enzymes with high affinities for both cAMP and cGMP but much higher turnover rates for cAMP. PDE3B is relatively abundant in tissues that maintain energy homoeostasis. In adipocytes, PDE3B (phosphodiesterase 3B) is an important regulatory effector in signaling pathways controlled by insulin and cAMP-increasing hormones. Previous results from PDE3B-transgenic mice indicate that PDE3B, plays an important role in modulation of energy metabolism. PDE3B is also known as HcGIP1, CGMP-Inhibited 3′,5′-Cyclic Phosphodiesterase B, Cyclic GMP-Inhibited Phosphodiesterase B, EC 3.1.4.17, CGI-PDE B, CGIP1, or Cyclic Nucleotide Phosphodiesterase.

An exemplary sequence of a human PDE3B mRNA transcript can be found at, for example, GenBank Accession No. GI: 1889438535 (NM_001363570.2; SEQ ID NO:201; reverse complement, SEQ ID NO: 202). The sequence of mouse PDE3B mRNA can be found at, for example, GenBank Accession No. GI: 112983647 (NM_011055.2; SEQ ID NO:203; reverse complement, SEQ ID NO: 204). The sequence of rat PDE3B mRNA can be found at, for example, GenBank Accession No. GI: 1939401976 (NM_017229.2; SEQ ID NO:205; reverse complement, SEQ ID NO: 206). The sequence of Macaca fascicularis PDE3B mRNA can be found at, for example, GenBank Accession No. GI: 982294968 (XM_005578550.2; SEQ ID NO: 207; reverse complement, SEQ ID NO: 208). The sequence of Macaca mulatta PDE3B mRNA can be found at, for example, GenBank Accession No. GI: 1622864110 (XM_015114810.2; SEQ ID NO: 209; reverse complement, SEQ ID NO: 210).

Additional examples of PDE3B mRNA sequences are readily available through publicly available databases, e.g., GenBank, UniProt, OMIM, UCSC Genome Browser, and the Macaca genome project web site. Further information on PDE3B can be found, for example, at www.ncbi.nlm.nih.gov/gene/?term=PDE3B.

The entire contents of each of the foregoing GenBank Accession numbers and the Gene database numbers are incorporated herein by reference as of the date of filing this application.

The term PDE3B, as used herein, also refers to variations of the PDE3B gene including variants provided in the SNP database. Numerous sequence variations within the PDE3B gene have been identified and may be found at, for example, NCBI dbSNP and UniProt (see, e.g., www.ncbi.nlm.nih.gov/snp/?term=PDE3B, the entire contents of which is incorporated herein by reference as of the date of filing this application.

As used herein, “Activin A Receptor Type 1C,” used interchangeably with the term “ACVR1C,” refers to a type I receptor for the TGFB family that mediates the activities of a diverse group of signaling molecules, including activin B, growth and differentiation factor 3 (GDF-3) and Nodal. Upon ligand binding, type I receptors phosphorylate cytoplasmic SMAD transcription factors, which then translocate to the nucleus and interact directly with DNA or in complex with other transcription factors. In rodents as well as humans, ALK7 expression is enriched in tissues that are important for the regulation of energy homeostasis, including adipose tissue, pancreatic islets, endocrine gut cells and the arcuate nucleus of the hypothalamus. In white adipose tissue, studies have shown that ALK7 signaling facilitates fat accumulation under conditions of nutrient overload, by repressing the expression of adrenergic receptors, thereby reducing catecholamine sensitivity. Accordingly, mutant mice lacking ALK7 globally, or only in adipocytes, are resistant to diet-induced obesity. Recent studies have identified polymorphic variants in the human Acvr1c gene which affect body fat distribution and protect from type II diabetes, indicating that ALK7 has very similar functions in humans as in rodents. ACVR1C is also known as ALK7, ACVRLK7, Activin Receptor-Like Kinase 7, EC 2.7.11.30, ACTR-IC, Activin Receptor Type IC, or EC 2.7.11.

An exemplary sequence of a human ACVR1C mRNA transcript can be found at, for example, GenBank Accession No. GI: 1519315475 (NM_145259.3; SEQ ID NO:211; reverse complement, SEQ ID NO: 212). The sequence of mouse ACVR1C mRNA can be found at, for example, GenBank Accession No. GI: 161333830 (NM_001111030.1; SEQ ID NO:213; reverse complement, SEQ ID NO: 214). The sequence of rat ACVR1C mRNA can be found at, for example, GenBank Accession No. GI: 1937875934 (NM_139090.2; SEQ ID NO:215; reverse complement, SEQ ID NO: 216). The sequence of Macaca fascicularis ACVR1C mRNA can be found at, for example, GenBank Accession No. GI: 982285785 (XM_005573224.2; SEQ ID NO: 217; reverse complement, SEQ ID NO: 218). The sequence of Macaca mulatta ACVR1C mRNA can be found at, for example, GenBank Accession No. GI: 388454445 (NM_001266690.1; SEQ ID NO: 219; reverse complement, SEQ ID NO: 220).

Additional examples of ACVR1C mRNA sequences are readily available through publicly available databases, e.g., GenBank, UniProt, OMIM, UCSC Genome Browser, and the Macaca genome project web site. Further information on ACVR1C can be found, for example, at www.ncbi.nlm.nih.gov/gene/?term=ACVR1C.

The entire contents of each of the foregoing GenBank Accession numbers and the Gene database numbers are incorporated herein by reference as of the date of filing this application.

The term ACVR1C, as used herein, also refers to variations of the ACVR1C gene including variants provided in the SNP database. Numerous sequence variations within the ACVR1C gene have been identified and may be found at, for example, NCBI dbSNP and UniProt (see, e.g., www.ncbi.nlm.nih.gov/gene/?term=ACVR1C, the entire contents of which is incorporated herein by reference as of the date of filing this application.

Specific exemplary target genes that mediate a skeletal muscle disorder include, but are not limited to, myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); and Double Homeobox 4 (DUX4).

As used herein, “myostatin,” used interchangeably with the term “MSTN,” refers to a secreted ligand of the TGF-beta (transforming growth factor-beta) superfamily of proteins. Ligands of this family bind various TGF-beta receptors leading to recruitment and activation of SMAD family transcription factors that regulate gene expression. The encoded preproprotein is proteolytically processed to generate each subunit of the disulfide-linked homodimer. This protein negatively regulates skeletal muscle cell proliferation and differentiation. Mutations in this gene are associated with increased skeletal muscle mass in humans and other mammals. Myostatin is also known as GDF8, Growth/Differentiation Factor 8, or MSLHP.

An exemplary sequence of a human myostatin mRNA transcript can be found at, for example, GenBank Accession No. GI: 1653961810 (NM_005259.3; SEQ ID NO:221; reverse complement, SEQ ID NO: 222). The sequence of mouse myostatin mRNA can be found at, for example, GenBank Accession No. GI: 922959927 (NM_010834.3; SEQ ID NO:223; reverse complement, SEQ ID NO: 224). The sequence of rat myostatin mRNA can be found at, for example, GenBank Accession No. GI: 9506906 (NM_019151.1; SEQ ID NO:225; reverse complement, SEQ ID NO: 226). The sequence of Macaca fascicularis myostatin mRNA can be found at, for example, GenBank Accession No. NM_001287623.1; SEQ ID NO: 227; reverse complement, SEQ ID NO: 228. The sequence of Macaca mulatta myostatin mRNA can be found at, for example, GenBank Accession No. GI: 121583757 (NM_001080119.1; SEQ ID NO: 229; reverse complement, SEQ ID NO: 230).

Additional examples of myostatin mRNA sequences are readily available through publicly available databases, e.g., GenBank, UniProt, OMIM, UCSC Genome Browser, and the Macaca genome project web site. Further information on myostatin can be found, for example, at www.ncbi.nlm.nih.gov/gene/?term=myostatin.

The entire contents of each of the foregoing GenBank Accession numbers and the Gene database numbers are incorporated herein by reference as of the date of filing this application.

The term myostatin, as used herein, also refers to variations of the myostatin gene including variants provided in the SNP database. Numerous sequence variations within the myostatin gene have been identified and may be found at, for example, NCBI dbSNP and UniProt (see, e.g., www.ncbi.nlm.nih.gov/gene/?term=myostatin, the entire contents of which is incorporated herein by reference as of the date of filing this application.

As used herein, “Cholinergic Receptor Nicotinic Alpha 1 Subunit,” used interchangeably with the term “CHRNA1,” refers to an alpha subunit of the muscle acetylcholine receptor (AChR). The muscle acetylcholine receptor consists of 5 subunits of 4 different types: 2 alpha subunits and 1 each of the beta, gamma, and delta subunits. This protein plays a role in acetlycholine binding/channel gating. After binding acetylcholine, the AChR responds by an extensive change in conformation that affects all subunits and leads to opening of an ion-conducting channel across the plasma membrane. CHRNA1 is associated with diseases associated such as Myasthenic Syndrome. CHRNA1 is also known as Cholinergic Receptor, Nicotinic, Alpha Polypeptide 1; Acetylcholine Receptor, Nicotinic, Alpha 1 (Muscle); ACHRA; CHRNA; Muscle Nicotinic Acetylcholine Receptor; CMS1A, CMS1B, CMS2A, FCCMS, SCCMS, or ACHRD.

An exemplary sequence of a human CHRNA1 mRNA transcript can be found at, for example, GenBank Accession No. GI: 1676317412 (NM_001039523.3; SEQ ID NO:231; reverse complement, SEQ ID NO: 232). The sequence of mouse CHRNA1 mRNA can be found at, for example, GenBank Accession No. GI: 425905338 (NM_007389.5; SEQ ID NO:233; reverse complement, SEQ ID NO: 234). The sequence of rat CHRNA1 mRNA can be found at, for example, GenBank Accession No. GI: 1937369362 (NM 024485.2; SEQ ID NO:235; reverse complement, SEQ ID NO: 236). The sequence of Macaca fascicularis CHRNA1 mRNA can be found at, for example, GenBank Accession No. GI: 982286285 (XM_015432377.1; SEQ ID NO: 237; reverse complement, SEQ ID NO: 238). The sequence of Macaca mulatta CHRNA1 mRNA can be found at, for example, GenBank Accession No. GI: 1622850381 (XM_001091711.4; SEQ ID NO: 239; reverse complement, SEQ ID NO: 240).

Additional examples of CHRNA1 mRNA sequences are readily available through publicly available databases, e.g., GenBank, UniProt, OMIM, UCSC Genome Browser, and the Macaca genome project web site. Further information on CHRNA1 can be found, for example, at www.ncbi.nlm.nih.gov/gene/?term=CHRNA1.

The entire contents of each of the foregoing GenBank Accession numbers and the Gene database numbers are incorporated herein by reference as of the date of filing this application.

The term CHRNA1, as used herein, also refers to variations of the CHRNA1 gene including variants provided in the SNP database. Numerous sequence variations within the CHRNA1 gene have been identified and may be found at, for example, NCBI dbSNP and UniProt (see, e.g., 1, the entire contents of which is incorporated herein by reference as of the date of filing this application.

As used herein, “Cholinergic Receptor Nicotinic Beta 1 Subunit,” used interchangeably with the term “CHRNB1,” refers to a beta subunit of the muscle acetylcholine receptor (AChR). The muscle acetylcholine receptor consists of 5 subunits of 4 different types: 2 alpha subunits and 1 each of the beta, gamma, and delta subunits. This protein plays a role in acetlycholine binding/channel gating. After binding acetylcholine, the AChR responds by an extensive change in conformation that affects all subunits and leads to opening of an ion-conducting channel across the plasma membrane. CHRNB1 is associated with diseases associated such as Myasthenic Syndrome. CHRNB1 is also known as Cholinergic Receptor, Nicotinic, Beta Polypeptide 1; Acetylcholine Receptor, Nicotinic, Beta 1 (Muscle); ACHRB; CHRNB; CMS1D, CMS2C, CMS2A, or SCCMS.

An exemplary sequence of a human CHRNB1 mRNA transcript can be found at, for example, GenBank Accession No. GI: 1519313560 (NM_000747.3; SEQ ID NO:241; reverse complement, SEQ ID NO: 242). The sequence of mouse CHRNB1 mRNA can be found at, for example, GenBank Accession No. GI: 160358781 (NM_009601.4; SEQ ID NO:243; reverse complement, SEQ ID NO: 244). The sequence of rat CHRNB1 mRNA can be found at, for example, GenBank Accession No. GI: 2048631755 (NM 001395118.1; SEQ ID NO:245; reverse complement, SEQ ID NO: 246). The sequence of Macaca fascicularis CHRNB1 mRNA can be found at, for example, GenBank Accession No. GI: 982302904 (XM_005582753.2; SEQ ID NO: 247; reverse complement, SEQ ID NO: 248). The sequence of Macaca mulatta CHRNB1 mRNA can be found at, for example, GenBank Accession No. GI: 1622877217 (XM_015118481.2; SEQ ID NO: 249; reverse complement, SEQ ID NO: 250).

Additional examples of CHRNB1 mRNA sequences are readily available through publicly available databases, e.g., GenBank, UniProt, OMIM, UCSC Genome Browser, and the Macaca genome project web site. Further information on CHRNB1 can be found, for example, at www.ncbi.nlm.nih.gov/gene/?term=CHRNB1.

The entire contents of each of the foregoing GenBank Accession numbers and the Gene database numbers are incorporated herein by reference as of the date of filing this application.

The term CHRNB1, as used herein, also refers to variations of the CHRNB1 gene including variants provided in the SNP database. Numerous sequence variations within the CHRNB1 gene have been identified and may be found at, for example, NCBI dbSNP and UniProt (see, e.g., www.ncbi.nlm.nih.gov/gene/?term=CHRNB1, the entire contents of which is incorporated herein by reference as of the date of filing this application.

As used herein, “Cholinergic Receptor Nicotinic Delta Subunit,” used interchangeably with the term “CHRND,” refers to a delta subunit of the muscle acetylcholine receptor (AChR). The muscle acetylcholine receptor consists of 5 subunits of 4 different types: 2 alpha subunits and 1 each of the beta, gamma, and delta subunits. After binding acetylcholine, the AChR responds by an extensive change in conformation that affects all subunits and leads to opening of an ion-conducting channel across the plasma membrane. CHRND is associated with diseases associated such as Myasthenic Syndrome. CHRND is also known as ACHRD, Cholinergic Receptor, Nicotinic, Delta Polypeptide; Acetylcholine Receptor, Nicotinic, Delta (Muscle); CMS2A; CMS3A, CMS3B, CMS3C, FCCMS, or SCCMS.

An exemplary sequence of a human CHRND mRNA transcript can be found at, for example, GenBank Accession No. GI: 1519243557 (NM_000751.3; SEQ ID NO:251; reverse complement, SEQ ID NO: 252). The sequence of mouse CHRND mRNA can be found at, for example, GenBank Accession No. GI: 426214082 (NM_021600.3; SEQ ID NO:253; reverse complement, SEQ ID NO: 254). The sequence of rat CHRND mRNA can be found at, for example, GenBank Accession No. GI: 9506486 (NM_019298.1; SEQ ID NO:255; reverse complement, SEQ ID NO: 256). The sequence of Macaca fascicularis CHRND mRNA can be found at, for example, GenBank Accession No. GI: 982288086 (XM_005574618.2; SEQ ID NO: 257; reverse complement, SEQ ID NO: 258). The sequence of Macaca mulatta CHRND mRNA can be found at, for example, GenBank Accession No. GI: 1622852529 (XM_028831231.1; SEQ ID NO: 259; reverse complement, SEQ ID NO: 260).

Additional examples of CHRND mRNA sequences are readily available through publicly available databases, e.g., GenBank, UniProt, OMIM, UCSC Genome Browser, and the Macaca genome project web site. Further information on CHRND can be found, for example, at www.ncbi.nlm.nih.gov/gene/?term=CHRND.

The entire contents of each of the foregoing GenBank Accession numbers and the Gene database numbers are incorporated herein by reference as of the date of filing this application.

The term CHRND, as used herein, also refers to variations of the CHRND gene including variants provided in the SNP database. Numerous sequence variations within the CHRND gene have been identified and may be found at, for example, NCBI dbSNP and UniProt (see, e.g., www.ncbi.nlm.nih.gov/gene/?term=CHRND, the entire contents of which is incorporated herein by reference as of the date of filing this application.

As used herein, “Cholinergic Receptor Nicotinic Epsilon Subunit,” used interchangeably with the term “CHRNE,” refers to a subunit of the acetylcholine receptor. Acetylcholine receptors at mature mammalian neuromuscular junctions are pentameric protein complexes composed of four subunits in the ratio of two alpha subunits to one beta, one epsilon, and one delta subunit. The acetylcholine receptor changes subunit composition shortly after birth when the epsilon subunit replaces the gamma subunit seen in embryonic receptors. Mutations in the epsilon subunit are associated with congenital myasthenic syndrome. CHRNE is also known as Cholinergic Receptor, Nicotinic, Epsilon; Acetylcholine Receptor, Nicotinic, Epsilon; ACHRE; CMS1D, CMS1E, CMS2A, CMS4A, CMS4B, CMS4C, FCCMS, or SCCMS.

An exemplary sequence of a human CHRNE mRNA transcript can be found at, for example, GenBank Accession No. GI: 1433531118 (NM_000080.4; SEQ ID NO: 261; reverse complement, SEQ ID NO: 262). The sequence of mouse CHRNE mRNA can be found at, for example, GenBank Accession No. GI: 6752949 (NM_009603.1; SEQ ID NO: 263; reverse complement, SEQ ID NO: 264). The sequence of rat CHRNE mRNA can be found at, for example, GenBank Accession No. GI: 8393128 (NM_017194.1; SEQ ID NO: 265; reverse complement, SEQ ID NO: 266). The sequence of Macaca fascicularis CHRNE mRNA can be found at, for example, GenBank Accession No. GI: 982302635 (XM_015437499.1; SEQ ID NO: 267; reverse complement, SEQ ID NO: 268). The sequence of Macaca mulatta CHRNE mRNA can be found at, for example, GenBank Accession No. GI: 1622876897 (XM_015118354.2; SEQ ID NO: 269; reverse complement, SEQ ID NO: 270).

Additional examples of CHRNE mRNA sequences are readily available through publicly available databases, e.g., GenBank, UniProt, OMIM, UCSC Genome Browser, and the Macaca genome project web site. Further information on CHRNE can be found, for example, at www.ncbi.nlm.nih.gov/gene/?term=CHRNE.

The entire contents of each of the foregoing GenBank Accession numbers and the Gene database numbers are incorporated herein by reference as of the date of filing this application.

The term CHRNE, as used herein, also refers to variations of the CHRNE gene including variants provided in the SNP database. Numerous sequence variations within the CHRNE gene have been identified and may be found at, for example, NCBI dbSNP and UniProt (see, e.g., www.ncbi.nlm.nih.gov/gene/?term=CHRNE, the entire contents of which is incorporated herein by reference as of the date of filing this application.

As used herein, “Cholinergic Receptor Nicotinic Gamma Subunit,” used interchangeably with the term “CHRNG,” refers to a subunit of the acetylcholine receptor. The mammalian muscle-type acetylcholine receptor is a transmembrane pentameric glycoprotein with two alpha subunits, one beta, one delta, and one epsilon (in adult skeletal muscle) or gamma (in fetal and denervated muscle) subunit. This gene, which encodes the gamma subunit, is expressed prior to the thirty-third week of gestation in humans. The gamma subunit of the acetylcholine receptor plays a role in neuromuscular organogenesis and ligand binding and disruption of gamma subunit expression prevents the correct localization of the receptor in cell membranes. Mutations in the subunit are associated with congenital myasthenic syndrome. CHRNG is also known as Cholinergic Receptor, Nicotinic, Gamma; Acetylcholine Receptor, Nicotinic, Gamma; or ACHRG.

An exemplary sequence of a human CHRNG mRNA transcript can be found at, for example, GenBank Accession No. GI: 1441481359 (NM_005199.5; SEQ ID NO: 271; reverse complement, SEQ ID NO: 272). The sequence of mouse CHRNG mRNA can be found at, for example, GenBank Accession No. GI: 119964695 (NM_009604.3; SEQ ID NO: 273; reverse complement, SEQ ID NO: 274). The sequence of rat CHRNG mRNA can be found at, for example, GenBank Accession No. GI: 9506488 (NM_019145.1; SEQ ID NO: 275; reverse complement, SEQ ID NO: 276). The sequence of Macaca fascicularis CHRNG mRNA can be found at, for example, GenBank Accession No. GI: 982288092 (XM_005574625.2; SEQ ID NO: 277; reverse complement, SEQ ID NO: 278). The sequence of Macaca mulatta CHRNG mRNA can be found at, for example, GenBank Accession No. GI: 1622852538 (XM_028831233.1; SEQ ID NO: 279; reverse complement, SEQ ID NO: 280).

Additional examples of CHRNG mRNA sequences are readily available through publicly available databases, e.g., GenBank, UniProt, OMIM, UCSC Genome Browser, and the Macaca genome project web site. Further information on CHRNG can be found, for example, at www.ncbi.nlm.nih.gov/gene/?term=CHRNG.

The entire contents of each of the foregoing GenBank Accession numbers and the Gene database numbers are incorporated herein by reference as of the date of filing this application.

The term CHRNG, as used herein, also refers to variations of the CHRNG gene including variants provided in the SNP database. Numerous sequence variations within the CHRNG gene have been identified and may be found at, for example, NCBI dbSNP and UniProt (see, e.g., www.ncbi.nlm.nih.gov/gene/?term=CHRNG, the entire contents of which is incorporated herein by reference as of the date of filing this application.

As used herein, “Collagen Type XIII Alpha 1 Chain,” used interchangeably with the term “COL13A1,” refers to a synaptic extracellular-matrix protein involved in the formation and maintenance of the neuromuscular synapse. COL13A1 encodes the collagen type XIII alpha1 chain (COL13A1), which is a single-pass type II transmembrane protein made of a short intracellular domain, a single transmembrane domain, and a triple-helical collagenous ectodomain. Studies have shown that patients with COL13A1 mutations underlie a myasthenic syndrome characterized by early onset muscle weakness with predominantly feeding and breathing difficulties often requiring ventilation and artificial feeding. COL13A1 is also known as COLXIIIA1, Collagen Alpha-1(XIII) Chain, or CMS19.

An exemplary sequence of a human COL13A1 mRNA transcript can be found at, for example, GenBank Accession No. GI: 1677498641 (NM_001130103.2; SEQ ID NO: 281; reverse complement, SEQ ID NO: 282). The sequence of mouse COL13A1 mRNA can be found at, for example, GenBank Accession No. GI: 755571593 (NM_007731.3; SEQ ID NO: 283; reverse complement, SEQ ID NO: 284). The sequence of rat COL13A1 mRNA can be found at, for example, GenBank Accession No. GI: 157821424 (NM_001109172.1; SEQ ID NO: 285; reverse complement, SEQ ID NO: 286). The sequence of Macaca fascicularis COL13A1 mRNA can be found at, for example, GenBank Accession No. GI: 982269148 (XM_015456252.1; SEQ ID NO: 287; reverse complement, SEQ ID NO: 288). The sequence of Macaca mulatta COL13A1 mRNA can be found at, for example, GenBank Accession No. GI: 1622966101 (XM_015147482.2; SEQ ID NO: 289; reverse complement, SEQ ID NO: 290).

Additional examples of COL13A1 mRNA sequences are readily available through publicly available databases, e.g., GenBank, UniProt, OMIM, UCSC Genome Browser, and the Macaca genome project web site. Further information on COL13A1 can be found, for example, at www.ncbi.nlm.nih.gov/gene/?term=COL13A1.

The entire contents of each of the foregoing GenBank Accession numbers and the Gene database numbers are incorporated herein by reference as of the date of filing this application.

The term COL13A1, as used herein, also refers to variations of the COL13A1 gene including variants provided in the SNP database. Numerous sequence variations within the COL13A1 gene have been identified and may be found at, for example, NCBI dbSNP and UniProt (see, e.g., www.ncbi.nlm.nih.gov/gene/?term=COL13A1 the entire contents of which is incorporated herein by reference as of the date of filing this application.

As used herein, “Docking Protein 7,” used interchangeably with the term “DOK7,” refers to a protein that is essential for neuromuscular synaptogenesis. The protein functions in aneural activation of muscle-specific receptor kinase, which is required for postsynaptic differentiation, and in the subsequent clustering of the acetylcholine receptor in myotubes. This protein can also induce autophosphorylation of muscle-specific receptor kinase. Mutations in this gene are a cause of congenital myasthenic syndrome. DOK7 is also known as C4orf25, Downstream Of Tyrosine Kinase 7, FLJ33718, FLJ39137, Chromosome 4 Open Reading Frame 25, CMS10, CMS1B, or FADS3.

An exemplary sequence of a human DOK7 mRNA transcript can be found at, for example, GenBank Accession No. GI: 1519242777 (NM_173660.5; SEQ ID NO: 291; reverse complement, SEQ ID NO: 292). The sequence of mouse DOK7 mRNA can be found at, for example, GenBank Accession No. GI: 1143077055 (NM_001348478.1; SEQ ID NO: 293; reverse complement, SEQ ID NO: 294). The sequence of rat DOK7 mRNA can be found at, for example, GenBank Accession No. GI: 194240570 (NM_001130062.1; SEQ ID NO: 295; reverse complement, SEQ ID NO: 296). The sequence of Macaca fascicularis DOK7 mRNA can be found at, for example, GenBank Accession No. GI: 982247946 (XM_015450057.1; SEQ ID NO: 297; reverse complement, SEQ ID NO: 298). The sequence of Macaca mulatta DOK7 mRNA can be found at, for example, GenBank Accession No. GI: 1622938489 (XM_015137905.2; SEQ ID NO: 299; reverse complement, SEQ ID NO: 300).

Additional examples of DOK7 mRNA sequences are readily available through publicly available databases, e.g., GenBank, UniProt, OMIM, UCSC Genome Browser, and the Macaca genome project web site. Further information on DOK7 can be found, for example, at www.ncbi.nlm.nih.gov/gene/?term=DOK7.

The entire contents of each of the foregoing GenBank Accession numbers and the Gene database numbers are incorporated herein by reference as of the date of filing this application.

The term DOK7, as used herein, also refers to variations of the DOK7 gene including variants provided in the SNP database. Numerous sequence variations within the DOK7 gene have been identified and may be found at, for example, NCBI dbSNP and UniProt (see, e.g., www.ncbi.nlm.nih.gov/gene/?term=DOK7, the entire contents of which is incorporated herein by reference as of the date of filing this application.

As used herein, “LDL Receptor Related Protein 4,” used interchangeably with the term “LRP4,” refers to a member of the low-density lipoprotein receptor-related protein family. LRP4 is a single-transmembrane protein that possesses a large extracellular domain with multiple LDLR repeats, EGF-like and β-propeller repeats; a transmembrane domain; and a short C-terminal region without an identifiable catalytic motif Mice lacking LRP4 die at birth and do not form the NMJ, indicating a critical role in neuromuscular junction (NMJ) formation. LPR4 mutation or malfunction is implicated in disorders including congenital myasthenic syndrome, myasthenia gravis, and diseases of bone or kidney. LRP4 is also known as MEGF7, LRP-4, SOST2, CLSS, Low-Density Lipoprotein Receptor-Related Protein 4, Multiple Epidermal Growth Factor-Like Domains 7, LRP10, KIAA0816, or CMS17.

An exemplary sequence of a human LRP4 mRNA transcript can be found at, for example, GenBank Accession No. GI: 1519312025 (NM_002334.4; SEQ ID NO: 301; reverse complement, SEQ ID NO: 302). The sequence of mouse LRP4 mRNA can be found at, for example, GenBank Accession No. GI: 224994222 (NM_172668.3; SEQ ID NO: 303; reverse complement, SEQ ID NO: 304). The sequence of rat LRP4 mRNA can be found at, for example, GenBank Accession No. GI: 329112575 (NM_031322.3; SEQ ID NO: 305; reverse complement, SEQ ID NO: 306). The sequence of Macaca fascicularis LRP4 mRNA can be found at, for example, GenBank Accession No. GI: 982294148 (XM_005578015.2; SEQ ID NO: 307; reverse complement, SEQ ID NO: 308). The sequence of Macaca mulatta LRP4 mRNA can be found at, for example, GenBank Accession No. GI: 1622863351 (XM_015114355.2; SEQ ID NO: 309; reverse complement, SEQ ID NO: 310).

Additional examples of LRP4 mRNA sequences are readily available through publicly available databases, e.g., GenBank, UniProt, OMIM, UCSC Genome Browser, and the Macaca genome project web site. Further information on LRP4 can be found, for example, at www.ncbi.nlm.nih.gov/gene/?term=LRP4.

The entire contents of each of the foregoing GenBank Accession numbers and the Gene database numbers are incorporated herein by reference as of the date of filing this application.

The term LRP4, as used herein, also refers to variations of the LRP4 gene including variants provided in the SNP database. Numerous sequence variations within the LRP4 gene have been identified and may be found at, for example, NCBI dbSNP and UniProt (see, e.g., www.ncbi.nlm.nih.gov/snp/?term=LRP4, the entire contents ofwhich is incorporated herein by reference as of the date of filing this application.

As used herein, “Muscle Associated Receptor Tyrosine Kinase,” used interchangeably with the term “MUSK,” refers to a muscle-specific tyrosine kinase receptor, which plays a central role in the formation and the maintenance of the neuromuscular junction (NMJ), the synapse between the motor neuron and the skeletal muscle. Recruitment of AGRIN by LRP4 to the MUSK signaling complex induces phosphorylation and activation of MUSK, the kinase of the complex. The activation of MUSK in myotubes regulates the formation of NMJs through the regulation of different processes including the specific expression of genes in subsynaptic nuclei, the reorganization of the actin cytoskeleton and the clustering of the acetylcholine receptors in the postsynaptic membrane. Mutations in this gene have been associated with congenital myasthenic syndrome. MUSK is also known as EC 2.7.10.1, FADS1, CMS9, FADS, Muscle, Skeletal Receptor Tyrosine-Protein Kinase, or Muscle-Specific Kinase Receptor.

An exemplary sequence of a human MUSK mRNA transcript can be found at, for example, GenBank Accession No. GI: 1609044119 (NM_005592.4; SEQ ID NO: 311; reverse complement, SEQ ID NO: 312). The sequence of mouse MUSK mRNA can be found at, for example, GenBank Accession No. GI: 260267047 (NM_001037127.2; SEQ ID NO: 313; reverse complement, SEQ ID NO: 314). The sequence of rat MUSK mRNA can be found at, for example, GenBank Accession No. GI: 1937920431 (NM 031061.2; SEQ ID NO: 315; reverse complement, SEQ ID NO: 316). The sequence of Macaca fascicularis MUSK mRNA can be found at, for example, GenBank Accession No. GI: 982300549 (XM_005581093.2; SEQ ID NO: 317; reverse complement, SEQ ID NO: 318). The sequence of Macaca mulatta MUSK mRNA can be found at, for example, GenBank Accession No. GI: 1622871800 (XM_015117113.2; SEQ ID NO: 319; reverse complement, SEQ ID NO: 320).

Additional examples of MUSK mRNA sequences are readily available through publicly available databases, e.g., GenBank, UniProt, OMIM, UCSC Genome Browser, and the Macaca genome project web site. Further information on MUSK can be found, for example, at www.ncbi.nlm.nih.gov/gene/?term=MUSK.

The entire contents of each of the foregoing GenBank Accession numbers and the Gene database numbers are incorporated herein by reference as of the date of filing this application.

The term MUSK, as used herein, also refers to variations of the MUSK gene including variants provided in the SNP database. Numerous sequence variations within the MUSK gene have been identified and may be found at, for example, NCBI dbSNP and UniProt (see, e.g., www.ncbi.nlm.nih.gov/gene/?term=MUSK, the entire contents of which is incorporated herein by reference as of the date of filing this application.

As used herein, “Receptor Associated Protein Of The Synapse,” used interchangeably with the term “RAPSN,” refers to a member of a family of proteins that are receptor associated proteins of the synapse. The encoded protein contains a conserved cAMP-dependent protein kinase phosphorylation site, and plays a critical role in clustering and anchoring nicotinic acetylcholine receptors at synaptic sites by linking the receptors to the underlying postsynaptic cytoskeleton, possibly by direct association with actin or spectrin. Mutations in this gene may play a role in postsynaptic congenital myasthenic syndromes. RAPSN is also known as RNF205, 43 KDa Receptor-Associated Protein Of The Synapse, RING Finger Protein 205, CMS1D, CMS1E, Acetylcholine Receptor-Associated 43 Kda Protein, RAPSYN, CMS11, CMS4C, FADS2, or FADS.

An exemplary sequence of a human RAPSN mRNA transcript can be found at, for example, GenBank Accession No. GI: 1519241818 (NM_005055.5; SEQ ID NO: 321; reverse complement, SEQ ID NO: 322). The sequence of mouse RAPSN mRNA can be found at, for example, GenBank Accession No. GI: 224967080 (NM_009023.3; SEQ ID NO: 323; reverse complement, SEQ ID NO: 324). The sequence of rat RAPSN mRNA can be found at, for example, GenBank Accession No. GI: 157819696 (NM_001108584.1; SEQ ID NO: 325; reverse complement, SEQ ID NO: 326). The sequence of Macaca fascicularis RAPSN mRNA can be found at, for example, GenBank Accession No. GI: 982294016 (XM_015434747.1; SEQ ID NO: 327; reverse complement, SEQ ID NO: 328). The sequence of Macaca mulatta RAPSN mRNA can be found at, for example, GenBank Accession No. GI: 1622863236 (XM_015114296.2; SEQ ID NO: 329; reverse complement, SEQ ID NO: 330).

Additional examples of RAPSN mRNA sequences are readily available through publicly available databases, e.g., GenBank, UniProt, OMIM, UCSC Genome Browser, and the Macaca genome project web site. Further information on RAPSN can be found, for example, at www.ncbi.nlm.nih.gov/gene/?term=RAPSN.

The entire contents of each of the foregoing GenBank Accession numbers and the Gene database numbers are incorporated herein by reference as of the date of filing this application.

The term RAPSN, as used herein, also refers to variations of the RAPSN gene including variants provided in the SNP database. Numerous sequence variations within the RAPSN gene have been identified and may be found at, for example, NCBI dbSNP and UniProt (see, e.g., www.ncbi.nlm.nih.gov/gene/?term=RAPSN, the entire contents of which is incorporated herein by reference as of the date of filing this application.

As used herein, “Sodium Voltage-Gated Channel Alpha Subunit 4,” used interchangeably with the term “SCN4A,” refers to a member of the voltage-gated sodium channel family. Voltage-gated sodium channels are transmembrane glycoprotein complexes composed of a large alpha subunit with 24 transmembrane domains and one or more regulatory beta subunits. They are responsible for the generation and propagation of action potentials in neurons and muscle. This gene encodes one member of the sodium channel alpha subunit gene family. It is expressed in skeletal muscle, and mutations in this gene have been linked to congenital myasthenic syndrome, and several myotonia and periodic paralysis disorders. SCN4A is also known as SkM1, Nav1.4, HYPP, Sodium Channel Protein Skeletal Muscle Subunit Alpha, Voltage-Gated Sodium Channel Subunit Alpha Nav1.4, HYKPP, Skeletal Muscle Voltage-Dependent Sodium Channel Type IV Alpha Subunit, CTC-264K15.6, Na(V)1.4, HOKPP2, CMS16, or NAC1A.

An exemplary sequence of a human SCN4A mRNA transcript can be found at, for example, GenBank Accession No. GI: 93587341 (NM_000334.4; SEQ ID NO: 331; reverse complement, SEQ ID NO: 332). The sequence of mouse SCN4A mRNA can be found at, for example, GenBank Accession No. GI: 134948031 (NM_133199.2; SEQ ID NO: 333; reverse complement, SEQ ID NO: 334). The sequence of rat SCN4A mRNA can be found at, for example, GenBank Accession No. GI: 1937369400 (NM_013178.2; SEQ ID NO: 335; reverse complement, SEQ ID NO: 336). The sequence of Macaca fascicularis SCN4A mRNA can be found at, for example, GenBank Accession No. GI: 982306407 (XM_015438708.1; SEQ ID NO: 337; reverse complement, SEQ ID NO: 338).

The sequence of Macaca mulatta SCN4A mRNA can be found at, for example, GenBank Accession No. GI: 1622880585 (XM_015120096.2; SEQ ID NO: 339; reverse complement, SEQ ID NO: 340).

Additional examples of SCN4A mRNA sequences are readily available through publicly available databases, e.g., GenBank, UniProt, OMIM, UCSC Genome Browser, and the Macaca genome project web site. Further information on SCN4A can be found, for example, at www.ncbi.nlm.nih.gov/gene/?term=SCN4A.

The entire contents of each of the foregoing GenBank Accession numbers and the Gene database numbers are incorporated herein by reference as of the date of filing this application.

The term SCN4A, as used herein, also refers to variations of the SCN4A gene including variants provided in the SNP database. Numerous sequence variations within the SCN4A gene have been identified and may be found at, for example, NCBI dbSNP and UniProt (see, e.g., www.ncbi.nlm.nih.gov/snp/?term=SCN4A, the entire contents of which is incorporated herein by reference as of the date of filing this application.

As used herein, “Double Homeobox 4,” used interchangeably with the term “DUX4,” refers to a transcriptional activator of many genes. DUX4 is normally expressed during early embryonic development, and is then effectively silenced in all tissues except the testis and thymus. DUX4 has been implicated as being involved in cell death, oxidative stress, muscle differentiation and growth, epigenetic regulation, and a number of other signaling pathways in skeletal muscle. Inappropriate expression of DUX4 in muscle cells is the cause of facioscapulohumeral muscular dystrophy (FSHD), which is characterized by muscle weakness and wasting (atrophy) that worsens slowly over time. DUX4 is also known as Double Homeobox Protein 10, Double Homeobox Protein 4, Double Homeobox Protein 4/10, DUX4L, and DUX10.

An exemplary sequence of a human DUX4 mRNA transcript can be found at, for example, GenBank Accession No. GI: 1774753171 (NM_001306068.3; SEQ ID NO: 341; reverse complement, SEQ ID NO: 342). The sequence of mouse DUX4 mRNA can be found at, for example, GenBank Accession No. GI: 126432555 (NM_001081954.1; SEQ ID NO: 343; reverse complement, SEQ ID NO: 344). The sequence of rat DUX4 mRNA can be found at, for example, GenBank Accession No. GI: 1958689769 (XM_008771031.3; SEQ ID NO: 345; reverse complement, SEQ ID NO: 346). The sequence of Macaca mulatta DUX4 mRNA can be found at, for example, GenBank Accession No. GI: 1622942424 (XM_028848991.1; SEQ ID NO: 347; reverse complement, SEQ ID NO: 348).

Additional examples of DUX4 mRNA sequences are readily available through publicly available databases, e.g., GenBank, UniProt, OMIM, UCSC Genome Browser, and the Macaca genome project web site. Further information on DUX4 can be found, for example, at www.ncbi.nlm.nih.gov/gene/?term=DUX4.

The entire contents of each of the foregoing GenBank Accession numbers and the Gene database numbers are incorporated herein by reference as of the date of filing this application.

The term DUX4, as used herein, also refers to variations of the DUX4 gene including variants provided in the SNP database. Numerous sequence variations within the DUX4 gene have been identified and may be found at, for example, NCBI dbSNP and UniProt (see, e.g., www.ncbi.nlm.nih.gov/gene/?term=DUX4, the entire contents of which is incorporated herein by reference as of the date of filing this application.

As used herein, “phospholamban,” used interchangeably with the term “PLN,” refers to a crucial regulator of cardiac contractility. PLN is a major substrate for the cAMP-dependent protein kinase in cardiac muscle. The encoded protein is an inhibitor of cardiac muscle sarcoplasmic reticulum Ca(2+)-ATPase in the unphosphorylated state, but inhibition is relieved upon phosphorylation of the protein. The subsequent activation of the Ca(2+) pump leads to enhanced muscle relaxation rates, thereby contributing to the inotropic response elicited in heart by beta-agonists. The encoded protein is a key regulator of cardiac diastolic function. Mutations in this gene are a cause of inherited human dilated cardiomyopathy with refractory congestive heart failure, and also familial hypertrophic cardiomyopathy. PLN is also known as CMD1P, PLB, Cardiac Phospholamban, or CMH.

An exemplary sequence of a human PLN mRNA transcript can be found at, for example, GenBank Accession No. GI: 1519242997 (NM_002667.5; SEQ ID NO: 349; reverse complement, SEQ ID NO: 350). The sequence of mouse PLN mRNA can be found at, for example, GenBank Accession No. GI: 213512815 (NM_001141927.1; SEQ ID NO: 351; reverse complement, SEQ ID NO: 352). The sequence of rat PLN mRNA can be found at, for example, GenBank Accession No. GI: 399124783 (NM_022707.2; SEQ ID NO: 353; reverse complement, SEQ ID NO: 354). The sequence of Macaca mulatta PLN mRNA can be found at, for example, GenBank Accession No. GI: 1863319929 (NM_001190894.2; SEQ ID NO: 355; reverse complement, SEQ ID NO: 356).

Additional examples of PLN mRNA sequences are readily available through publicly available databases, e.g., GenBank, UniProt, OMIM, UCSC Genome Browser, and the Macaca genome project web site. Further information on PLN can be found, for example, at www.ncbi.nlm.nih.gov/gene/?term=PLN.

The entire contents of each of the foregoing GenBank Accession numbers and the Gene database numbers are incorporated herein by reference as of the date of filing this application.

The term PLN, as used herein, also refers to variations of the PLN gene including variants provided in the SNP database. Numerous sequence variations within the PLN gene have been identified and may be found at, for example, NCBI dbSNP and UniProt (see, e.g., www.ncbi.nlm.nih.gov/snp/?term=PLN, the entire contents of which is incorporated herein by reference as of the date of filing this application.

As used herein, “calcium/calmodulin dependent protein kinase II delta,” used interchangeably with the term “CAMK2D,” refers to a member of the serine/threonine protein kinase family and the Ca(2+)/calmodulin-dependent protein kinase subfamily. CAMK2D is involved in the regulation of Ca(2+) homeostatis and excitation-contraction coupling in heart by targeting ion channels, transporters and accessory proteins involved in Ca(2+) influx into the myocyte, Ca(2+) release from the sarcoplasmic reticulum (SR), SR Ca(2+) uptake and Na(+) and K(+) channel transport. CAMK2D also targets transcription factors and signaling molecules to regulate heart function. In its activated form, CAMK2D is involved in the pathogenesis of dilated cardiomyopathy and heart failure. CAMK2D contributes to cardiac decompensation and heart failure by regulating SR Ca(2+) release via direct phosphorylation of RYR2 Ca(2+) channel. In the nucleus, CAMK2D phosphorylates the MEF2 repressor HDAC4, promoting its nuclear export and binding to 14-3-3 protein, and expression of MEF2 and genes involved in the hypertrophic program. CAMK2D is essential for left ventricular remodeling responses to myocardial infarction. In pathological myocardial remodeling, CAMK2D acts downstream of the beta adrenergic receptor signaling cascade to regulate key proteins involved in excitation-contraction coupling. CAMK2D regulates Ca(2+) influx to myocytes by binding and phosphorylating the L-type Ca(2+) channel subunit beta-2 CACNB2. In addition to Ca(2+) channels, CAMK2D can target and regulate the cardiac sarcolemmal Na(+) channel Nav1.5/SCN5A and the K+ channel Kv4.3/KCND3, which contribute to arrhythmogenesis in heart failure. CAMK2D phosphorylates phospholamban (PLN), an endogenous inhibitor of SERCA2A/ATP2A2, contributing to the enhancement of SR Ca(2+) uptake that may be important in frequency-dependent acceleration of relaxation and maintenance of contractile function during acidosis. CAMK2D may participate in the modulation of skeletal muscle function in response to exercise, by regulating SR Ca(2+) transport through phosphorylation of PLN and triadin, a ryanodine receptor-coupling factor. CAMK2D is also known as Calcium/Calmodulin-Dependent Protein Kinase Type II Delta Chain, CaM Kinase II Delta Subunit, CaM Kinase II Subunit Delta, CAMKD, EC 2.7.11.17, or EC 2.7.11.

An exemplary sequence of a human CAMK2D mRNA transcript can be found at, for example, GenBank Accession No. GI: 1519243899 (NM_001321571.2; SEQ ID NO: 357; reverse complement, SEQ ID NO: 358). The sequence of mouse CAMK2D mRNA can be found at, for example, GenBank Accession No. GI: 654824235 (NM_001025439.2; SEQ ID NO: 359; reverse complement, SEQ ID NO: 360). The sequence of rat CAMK2D mRNA can be found at, for example, GenBank Accession No. GI: 144922682 (NM_012519.2; SEQ ID NO: 361; reverse complement, SEQ ID NO: 362). The sequence of Macaca mulatta CAMK2D mRNA can be found at, for example, GenBank Accession No. GI: 1622941163 (XM_015139100.2; SEQ ID NO: 363; reverse complement, SEQ ID NO: 364).

Additional examples of CAMK2D mRNA sequences are readily available through publicly available databases, e.g., GenBank, UniProt, OMIM, UCSC Genome Browser, and the Macaca genome project web site. Further information on CAMK2D can be found, for example, at www.ncbi.nlm.nih.gov/gene/?term=CAMK2D.

The entire contents of each of the foregoing GenBank Accession numbers and the Gene database numbers are incorporated herein by reference as of the date of filing this application.

The term CAMK2D, as used herein, also refers to variations of the CAMK2D gene including variants provided in the SNP database. Numerous sequence variations within the CAMK2D gene have been identified and may be found at, for example, NCBI dbSNP and UniProt (see, e.g., www.ncbi.nlm.nih.gov/snp/?term=CAMK2D, the entire contents of which is incorporated herein by reference as of the date of filing this application.

In other embodiments, the antisense strand polynucleotides disclosed herein are substantially complementary to the target gene sequence and comprise a contiguous nucleotide sequence which is at least about 80% complementary over its entire length to the equivalent region of the nucleotide sequence of SEQ ID NOs: 1-4 for ADRB1, or a fragment of SEQ ID NOs: 1-4, such as about 85%, 86%, 87%, 88%, 89%, 90%, 91%, 92%, 93%, 94%, 95%, 96%, 97%, 98%, or about 99% complementary.

In other embodiments, the antisense polynucleotides disclosed herein are substantially complementary to the target ADRB1 sequence and comprise a contiguous nucleotide sequence which is at least about 80% complementary over its entire length to any one of the sense strand nucleotide sequences in any one of Tables 2-5, 7B, and 7C, and, or a fragment of any one of the sense strand nucleotide sequences in any one of Tables 2-5, 7B, and 7C, such as about 85%, 86%, 87%, 88%, 89%, 90%, 91%, 92%, 93%, 94%, 95%, 96%, 97%, 98%, or about 99% complementary.

In one embodiment, an RNAi agent of the disclosure includes a sense strand that is substantially complementary to an antisense polynucleotide which, in turn, is the same as a target ADRB1 sequence, and wherein the sense strand polynucleotide comprises a contiguous nucleotide sequence which is at least about 80% complementary over its entire length to the equivalent region of the nucleotide sequence of SEQ ID NOs: 5-8, or a fragment of any one of SEQ ID NOs: 5-8, such as about 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target ADRB1 sequence, and wherein the sense strand polynucleotide comprises a contiguous nucleotide sequence which is at least about 80% complementary over its entire length to any one of the antisense strand nucleotide sequences in any one of any one of Tables 2-5, 7B, and 7C, or a fragment of any one of the antisense strand nucleotide sequences in any one of Tables 2-5, 7B, and 7C, such as about 86%, 87%, 88%, 89%, 90%, 91%, 92%, 93%, 94%, 95%, 96%, 97%, 98%, or about 99% complementary.

In some embodiments, the antisense polynucleotides disclosed herein are substantially complementary to the target LEP sequence and comprise a contiguous nucleotide sequence which is at least about 80% complementary over its entire length to any one of the sense strand nucleotide sequences in any one of Tables 9-16, or a fragment of any one of the sense strand nucleotide sequences in any one of Tables 9-16, such as about 85%, 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target LEP sequence, and wherein the sense strand polynucleotide comprises a contiguous nucleotide sequence which is at least about 80% complementary over its entire length to any one of the antisense strand nucleotide sequences in any one of any one of Tables 9-16, or a fragment of any one of the antisense strand nucleotide sequences in any one of Tables 9-16, such as about 86%, 87%, 88%, 89%, 90%, 91%, 92%, 93%, 94%, 95%, 96%, 97%, 98%, or about 99% complementary.

In some embodiments, the antisense polynucleotides disclosed herein are substantially complementary to the target PLN sequence and comprise a contiguous nucleotide sequence which is at least about 80% complementary over its entire length to any one of the sense strand nucleotide sequences in any one of Tables 19-22, or a fragment of any one of the sense strand nucleotide sequences in any one of Tables 19-22, such as about 85%, 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target PLN sequence, and wherein the sense strand polynucleotide comprises a contiguous nucleotide sequence which is at least about 80% complementary over its entire length to any one of the antisense strand nucleotide sequences in any one of any one of Tables 19-22, or a fragment of any one of the antisense strand nucleotide sequences in any one of Tables 19-22, such as about 86%, 87%, 88%, 89%, 90%, 91%, 92%, 93%, 94%, 95%, 96%, 97%, 98%, or about 99% complementary.

In some embodiments, the antisense polynucleotides disclosed herein are substantially complementary to the target CAMK2D sequence and comprise a contiguous nucleotide sequence which is at least about 80% complementary over its entire length to any one of the sense strand nucleotide sequences in any one of Tables 23-26, or a fragment of any one of the sense strand nucleotide sequences in any one of Tables 23-26, such as about 85%, 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target CAMK2D sequence, and wherein the sense strand polynucleotide comprises a contiguous nucleotide sequence which is at least about 80% complementary over its entire length to any one of the antisense strand nucleotide sequences in any one of any one of Tables 23-26, or a fragment of any one of the antisense strand nucleotide sequences in any one of Tables 23-26, such as about 86%, 87%, 88%, 89%, 90%, 91%, 92%, 93%, 94%, 95%, 96%, 97%, 98%, or about 99% complementary.

In some embodiments, the double-stranded region of a double-stranded iRNA agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a double-stranded iRNA agent is equal to or at least 14, 15, 16, 17, 18, 19, 20, 21, 22, 23, 23, 24, 25, 26, 27, 28, 29, or 30 nucleotides in length.

In some embodiments, the sense strand of a double-stranded iRNA agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the double-stranded iRNA agent are each independently 15 to 30 nucleotides in length.

In one embodiment, the sense and antisense strands of the double-stranded iRNA agent are each independently 19 to 25 nucleotides in length.

In one embodiment, the sense and antisense strands of the double-stranded iRNA agent are each independently 21 to 23 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single stranded overhangs at the 3′-end.

In one aspect of the invention, an agent for use in the methods and compositions of the invention is a single-stranded antisense nucleic acid molecule that inhibits a target mRNA via an antisense inhibition mechanism. The single-stranded antisense RNA molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense RNA molecule may be about 15 to about 30 nucleotides in length and have a sequence that is complementary to a target sequence. For example, the single-stranded antisense RNA molecule may comprise a sequence that is at least about 15, 16, 17, 18, 19, 20, or more contiguous nucleotides from any one of the antisense sequences described herein.

In one embodiment, at least partial suppression of the expression of a target gene, is assessed by a reduction of the amount of target mRNA which can be isolated from or detected in a first cell or group of cells in which a target gene is transcribed and which has or have been treated such that the expression of a target gene is inhibited, as compared to a second cell or group of cells substantially identical to the first cell or group of cells but which has or have not been so treated (control cells). The degree of inhibition may be expressed in terms of:

$$\frac{( mRNA in control cells ) - ( mRNA in treated cells )}{( mRNA in control cells )} 100 \%$$

In one embodiment, inhibition of expression is determined by the dual luciferase method wherein the RNAi agent is present at 10 nM.

The phrase “contacting a cell with an RNAi agent,” such as a dsRNA, as used herein, includes contacting a cell by any possible means. Contacting a cell with an RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, or to the bloodstream or the subcutaneous space, such that the agent will subsequently reach the tissue where the cell to be contacted is located. In some embodiments, the RNAi agent may contain or be coupled to a ligand, e.g., one or more GalNAc derivatives as described below, that directs or otherwise stabilizes the RNAi agent at a site of interest, e.g., the liver. In other embodiments, the RNAi agent may contain or be coupled to one or more C₂₂ hydrocarbon chains and one or more GalNAc derivatives. In other embodiments, the RNAi agent contains or is coupled to one or more C₂₂ hydrocarbon chains and does not contain or is not coupled to one or more GalNAc derivatives. Combinations of in vitro and in vivo methods of contacting are also possible. For example, a cell may also be contacted in vitro with an RNAi agent and subsequently transplanted into a subject.

In one embodiment, contacting a cell with an RNAi agent includes “introducing” or “delivering the RNAi agent into the cell” by facilitating or effecting uptake or absorption into the cell. Absorption or uptake of a RNAi agent can occur through unaided diffusive or active cellular processes, or by auxiliary agents or devices. Introducing a RNAi agent into a cell may be in vitro or in vivo. For example, for in vivo introduction, a RNAi agent can be injected into a tissue site or administered systemically. In vitro introduction into a cell includes methods known in the art such as electroporation and lipofection. Further approaches are described herein below or are known in the art.

The term “lipid nanoparticle” or “LNP” is a vesicle comprising a lipid layer encapsulating a pharmaceutically active molecule, such as a nucleic acid molecule, e.g., a RNAi agent or a plasmid from which a RNAi agent is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), or a non-primate (such as a a cow, a pig, a horse, a goat, a rabbit, a sheep, a hamster, a guinea pig, a cat, a dog, a rat, or a mouse), or a bird that expresses the target gene, either endogenously or heterologously. In one embodiment, the subject is a human, such as a human being treated or assessed for a disease, disorder, or condition that would benefit from reduction in target gene expression; a human at risk for a disease, disorder, or condition that would benefit from reduction in target gene expression; a human having a disease, disorder, or condition that would benefit from reduction in target gene expression; or human being treated for a disease, disorder, or condition that would benefit from reduction in target gene expression as described herein. In some embodiments, the subject is a female human. In other embodiments, the subject is a male human. In one embodiment, the subject is an adult subject. In another embodiment, the subject is a pediatric subject.

As used herein, the terms “treating” or “treatment” refer to a beneficial or desired result including, but not limited to, alleviation or amelioration of one or more signs or symptoms associated with target gene expression or target gene protein production, e.g., a target gene-associated disease, e.g., a skeletal muscle disorder, a cardiac muscle disorder, or an adipose tissue disorder, or symptoms associated with unwanted target gene expression; diminishing the extent of unwanted target activation or stabilization; amelioration or palliation of unwanted target activation or stabilization. “Treatment” can also mean prolonging survival as compared to expected survival in the absence of treatment.

The term “lower” in the context of the level of a target gene in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of a target gene in a subject is a decrease to a level accepted as within the range of normal for an individual without such disorder. In certain embodiments, the expression of the target is normalized, i.e., decreased towards or to a level accepted as within the range of normal for an individual without such disorder, e.g., blood glucose level, blood uric acid level, blood lipid level, blood oxygen level, white blood cell count, kidney function, spleen function, liver function. For example, chronic hyperuricemia is defined as serum urate levels greater than 6.8 mg/dl (greater than 360 mmol/), the level above which the physiological saturation threshold is exceeded (Mandell, Cleve. Clin. Med. 75:S5-S8, 2008). As used here, “lower” in a subject can refer to lowering of gene expression or protein production in a cell in a subject does not require lowering of expression in all cells or tissues of a subject. For example, as used herein, lowering in a subject can include lowering of gene expression or protein production in a subject.

The term “lower” can also be used in association with normalizing a symptom of a disease or condition, i.e. decreasing the difference between a level in a subject suffering from a target gene-associated disease towards or to a level in a normal subject not suffering from a target gene-associated disease. As used herein, if a disease is associated with an elevated value for a symptom, “normal” is considered to be the upper limit of normal. If a disease is associated with a decreased value for a symptom, “normal” is considered to be the lower limit of normal.

As used herein, “prevention” or “preventing,” when used in reference to a disease, disorder, or condition thereof, that would benefit from a reduction in expression of a target gene or production of a target protein, refers to a reduction in the likelihood that a subject will develop a symptom associated with such a disease, disorder, or condition, e.g., a symptom of a target gene-associated disease. The failure to develop a disease, disorder, or condition, or the reduction in the development of a symptom associated with such a disease, disorder, or condition (e.g., by at least about 10% on a clinically accepted scale for that disease or disorder), or the exhibition of delayed symptoms delayed (e.g., by days, weeks, months or years) is considered effective prevention.

As used herein, the term “target gene-associated disease,” is a disease or disorder that would benefit from reduction in the expression or activity of the target gene. The term “target gene-associated disease,” is a disease or disorder that is caused by, or associated with expression or protein production of the target gene. The term “target gene-associated disease” includes a disease, disorder or condition that would benefit from a decrease in expression or protein activity of the target gene. Additional information regarding specific target genes and disease that would benefit from reduction in expression of the target gene are descried below.

In one embodiment, the target gene-associated disease is a cardiac muscle disease or disorder.

In one embodiment, the target gene-associated disease is a skeletal muscle disease or disorder.

In one embodiment, the target gene-associated disease is a adipose tissue disease or disorder.

Exemplary cardiac muscle disorders include obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); and hypertrophic cardiomyopathy (HCM).

“Heart failure” (“HF”) or “congestive heart failure” (“CHF”) is a chronic condition in which the heart doesn't pump blood as well as it should. Heart failure occurs when the heart's capacity to pump blood cannot keep up with the body's need. Heart failure can occur if the heart cannot pump (systolic) or fill (diastolic) adequately. As the heart weakens, blood begins to back up and force liquid through the capillary walls. The term “congestive” refers to the resulting buildup of fluid in the ankles and feet, arms, lungs, and/or other organs.

One type of heart failure is “heart failure with preserved left ventricular function” (“HF-pEF”) also known as “heart failure with preserved ejection fraction” (“HF-pEF”) is a condition in which the heart contracts and pumps normally, but the ventricles are thicker and stiffer than normal. Because of this, the ventricles can't relax properly and fill up all the way. Because there's less blood in the ventricles, less blood is pumped out to the rest of the body when the heart contracts.

The most common cause of congestive heart failure is coronary artery disease. Risk factors for coronary artery disease include high levels of cholesterol and/or triglyceride, high blood pressure, poor diet, a sedentary lifestyle, diabetes, smoking, being overweight or obese, and stress. In addition to coronary artery disease, several other conditions can damage the heart muscles, including inherited and genetic factors, some infections and autoimmune diseases and some treatments such as chemotherapy.

Symptoms of CHF include shortness of breath, fatigue, swollen legs, and rapid heartbeat.

Treatments can include eating less salt, limiting fluid intake, and taking prescription medications, e.g., vasodilators, diuretics, aldosterone inhibitors, ACE inhibitors or ARB drugs, digitalis glycosides, anticoagulants or antiplatelets, beta-blockers, and tranquilizers, and surgical procedures, include for example, bypass surgery, heart valve replacement, implantation of a pacemaker, e.g., biventricular pacing therapy or an implantable cardioverter defibrillator, ventricular assist devices (VAD therapy), and heart transplant.

“Hypertrophic cardiomyopathy” (“HCM”) refers to impaired heart function associated with abnormally thick heart muscle in the absence of other heart disease; e.g., valvular heart disease. “Hypertrophic obstructive cardiomyopathy” (“HOCM”) is a subtype of HCM, where the wall (septum) between the two bottom chambers of the heart thickens. The walls of the pumping chamber can also become stiff The thickened septum may cause a narrowing that can block or reduce the blood flow from the left ventricle to the aorta, which is a condition called “outflow tract obstruction.”

Both HCM and HOCM may be caused by heart muscle gene mutation, which may be inherited. As such, multiple family members may be affected by HCM and HOCM. Phenotypic expression of the gene mutation may be variable.

Both HCM and HOCM may be caused by heart muscle gene mutation, which may be inherited. As such, multiple family members may be affected by HCM and HOCM. Phenotypic expression of the gene mutation may be variable. In other words, even with the same gene mutation, the severity of heart function impairment may vary between affected patients.

Symptoms associated with HCM may vary in severity and character as well, including, fatigue, chest pain, dyspnea, abnormal heart rhythm, heart failure, syncope, and sudden cardiac death.

Treatments include pacemakers, defibrillators, alcohol septal ablation, surgical myectomy, advanced heart failure therapy, beta blockers, calcium channel blockers, and anti-arrhythmics.

“Familial hypertrophic cardiomyopathy” is an autosomal dominant disease characterized mainly by left ventricular hypertrophy. Thickening usually occurs in the interventricular septum. In some, thickening of the interventricular septum impedes the flow of oxygen-rich blood from the heart, which may lead to an abnormal heart sound during a heartbeat (heart murmur) and other signs and symptoms of the condition. Other affected individuals do not have physical obstruction of blood flow, but the pumping of blood is less efficient, which can also lead to symptoms of the condition. Cardiac hypertrophy often begins in adolescence or young adulthood, although it can develop at any time throughout life.

The symptoms of familial hypertrophic cardiomyopathy are variable, even within the same family. Many affected individuals have no symptoms. Other people with familial hypertrophic cardiomyopathy may experience chest pain; shortness of breath, especially with physical exertion; a sensation of fluttering or pounding in the chest (palpitations); lightheadedness; dizziness; and fainting.

While most people with familial hypertrophic cardiomyopathy are symptom-free or have only mild symptoms, this condition can have serious consequences. It can cause abnormal heart rhythms (arrhythmias) that may be life threatening. People with familial hypertrophic cardiomyopathy have an increased risk of sudden death, even if they have no other symptoms of the condition. A small number of affected individuals develop potentially fatal heart failure, which may require heart transplantation.

Mutations in one of several genes can cause familial hypertrophic cardiomyopathy; the most commonly involved genes are MYH7, MYBPC3, TNNT2, and TNNI3. Other genes, including some that have not been identified, may also be involved in this condition.

Treatments include, beta blockers, calcium channel blockers, heart rhythm drugs such as amiodarone (Pacerone) or disopyramide (Norpace), and blood thinners such as warfarin (Coumadin, Jantoven), dabigatran (Pradaxa), rivaroxaban (Xarelto) or apixaban (Eliquis). Surgeries or other procedures include apical myectomy, septal myectomy, septal ablation, and implantable cardioverter-defibrillator (ICD).

“Atrial fibrillation” (“AFIB”) is when the atria beat chaotically and irregularly—out of coordination with the ventricles. The result is a fast and irregular heart rhythm. The heart rate in atrial fibrillation may range from 100 to 175 beats a minute. The normal range for a heart rate is 60 to 100 beats a minute.

Episodes of atrial fibrillation may come and go, or may go away and may require treatment. Although atrial fibrillation itself usually isn't life-threatening, it is a serious medical condition that sometimes requires emergency treatment.

A major concern with atrial fibrillation is the potential to develop blood clots within the atria which may circulate to other organs and lead to blocked blood flow (ischemia).

Causes of AFIB include, abnormalities or damage to the heart's structure, high blood pressure, heart attack, coronary artery disease, abnormal heart valves, congenital heart defects, an overactive thyroid gland or other metabolic imbalance, exposure to stimulants, such as medications, caffeine, tobacco or alcohol, sick sinus syndrome—improper functioning of the heart's natural pacemaker, lung diseases, previous heart surgery, viral infections, stress due to surgery, pneumonia or other illnesses, and sleep apnea.

Symptoms include palpitations, which are sensations of a racing, uncomfortable, irregular heartbeat or a flip-flopping in the chest, weakness, reduced ability to exercise, fatigue, lightheadedness, dizziness, shortness of breath, and chest pain.

Treatments include, electrical cardioversion, anti-arrhythmics, digoxin, beta blockers, calcium channel blockers anticoagulants, catheter ablation, Maze procedure, atrioventricular (AV) node ablation, and left atrial appendage closure.

“Ventricular fibrillation” (“VFIB”) is a type of abnormal heart rhythm (arrhythmia). During ventricular fibrillation, disorganized heart signals cause the ventricles to twitch (quiver) uselessly. As a result, the heart doesn't pump blood to the rest of the body.

Ventricular fibrillation is an emergency that requires immediate medical attention. It's the most frequent cause of sudden cardiac death.

Collapse and loss of consciousness is the most common symptom of ventricular fibrillation. Other symptoms include chest pain, very fast heartbeat (tachycardia), dizziness, nausea, and shortness of breath.

Risk factors include previous episode of ventricular fibrillation, previous heart attack, a congenital heart defect, heart muscle disease (cardiomyopathy), injuries that cause damage to the heart muscle, such as being struck by lightning, drug misuse, especially with cocaine or methamphetamine, and severe imbalance of potassium or magnesium.

Treatments include, cardiopulmonary resuscitation (CPR), defibrillation, anti-arrhythmics, an implantable cardioverter-defibrillator (ICD), cardiac ablation, coronary angioplasty and stent placement, and coronary bypass surgery.

A “myocardial infarction” or “MI” occurs when the flow of blood to the heart is blocked. The blockage is most often a buildup of fat, cholesterol and other substances, which form a plaque in the arteries that feed the heart (coronary arteries).

Symptoms include pressure, tightness, pain, or a squeezing or aching sensation in the chest or arms that may spread to the neck, jaw or back, nausea, indigestion, heartburn or abdominal pain, shortness of breath, cold sweat, fatigue, lightheadedness or sudden dizziness

Heart attack risk factors include age (e.g., men age 45 or older and women age 55 or older are more likely to have a heart attack than are younger men and women, tobacco, high blood pressure. Over time, high blood pressure can damage arteries that lead to your heart. High blood pressure that occurs with other conditions, such as obesity, high cholesterol or diabetes, increases your risk even more, high cholesterol or triglyceride levels, obesity, diabetes, metabolic syndrome, family history of heart attacks, lack of physical activity, stress, illicit drug use, a history of preeclampsia, and an autoimmune condition.

Treatments include, aspirin, thrombolytics, antiplatelet agents, other blood-thinning medications, pain relievers, nitroglycerin, beta blockers, ACE inhibitors, statins, coronary angioplasty and stenting, and coronary artery bypass surgery.

“Supraventricular tachycardia” (“SVT”) is as an abnormally fast or erratic heartbeat that affects the heart's atria. During an episode of SVT, the heart beats about 150 to 220 times per minute, but it can occasionally beat faster or slower.

The main symptom of supraventricular tachycardia (SVT) is a very fast heartbeat (100 beats a minute or more) that may last for a few minutes to a few days. The fast heartbeat may come and go suddenly, with stretches of normal heart rates in between.

Signs and symptoms of supraventricular tachycardia may include very fast (rapid) heartbeat, a fluttering or pounding in the chest (palpitations), a pounding sensation in the neck, weakness or feeling very tired (fatigue), chest pain, shortness of breath, lightheadedness or dizziness, sweating, and fainting (syncope) or near fainting. Some with SVT have no signs or symptoms at all.

For some, a supraventricular tachycardia episode is related to an obvious trigger, such as exercise, stress or lack of sleep. Some people may not have a noticeable trigger. Things that may cause an SVT episode include age, coronary artery disease, previous heart surgery, heart disease, heart failure, other heart problems, such as Wolff-Parkinson-White syndrome, chronic lung disease, consuming too much caffeine, drinking too much alcohol, drug use, particularly stimulants such as cocaine and methamphetamines, pregnancy, smoking, thyroid disease, tobacco, sleep apnea, diabetes, and certain medications, including asthma medications and over-the-counter cold and allergy drugs.

Treatments include, carotid sinus massage, vagal maneuvers, cardioversion, beta blockers, anti-arrhythmics, calcium channel blocker, catheter ablation, and pacemaker.

“Hypertrophic cardiomyopathy” (“HCM”) is a disease in which the heart muscle becomes abnormally thick (hypertrophied). The thickened heart muscle can make it harder for the heart to pump blood.

“Angina” is a type of chest pain caused by reduced blood flow to the heart. Angina is a symptom of coronary artery disease.

Angina, also called angina pectoris, is often described as squeezing, pressure, heaviness, tightness or pain in your chest. Some with angina symptoms say angina feels like a vise squeezing their chest or a heavy weight lying on their chest. There may also be pain in the arms, neck, jaw, shoulder or back. Other symptoms that you may have with angina include dizziness, fatigue, nausea, shortness of breath, and sweating.

Risk factors include tobacco, diabetes, high blood pressure, high cholesterol or triglyceride levels, family history of heart disease, age (e.g., men older than 45 and women older than 55 have a greater risk than do younger adults), lack of exercise, obesity, and stress.

Treatments include, lifestyle changes, nitrates, aspirin, clot-preventing drugs, beta blockers, statins, calcium channel blockers, blood pressure-lowering medications, angiotensin-converting enzyme (ACE) inhibitors or angiotensin II receptor blockers (ARBs), ranolazine (Ranexa), angioplasty and stenting, coronary artery bypass surgery, and external counterpulsation (ECP).

Exemplary skeletal muscle disorders include Myostatin-related muscle hypertrophy, congenital myasthenic syndrome, and facioscapulohumeral muscular dystrophy (FSHD).

Myostatin-related muscle hypertrophy is a rare condition characterized by reduced body fat and increased muscle size. Affected individuals have up to twice the usual amount of muscle mass in their bodies. They also tend to have increased muscle strength. Myostatin-related muscle hypertrophy is caused by mutations in the MSTN gene. It follows an incomplete autosomal dominant pattern of inheritance.

Congenital myasthenic syndromes (CMS) are a heterogeneous group of early-onset genetic neuromuscular transmission disorders due to mutations in proteins involved in the organisation, maintenance, function, or modification of the motor endplate (endplate myopathies), e.g., CHRNA1, CHRNB1, CHRBD, CHRNE, CHRNG, COL13A1, DOX7, LRP4, MUSK, RAPSN, or SCN4A. CMS are clinically characterised by abnormal fatigability, or transient or permanent weakness of extra-ocular, facial, bulbar, truncal, respiratory, or limb muscles. Onset of endplate myopathy is intrauterine, congenital, in infancy, or childhood, and rarely in adolescence. Severity ranges from mild, phasic weakness, to disabling, permanent muscle weakness, respiratory insufficiency, and early death. All subtypes of CMS share the clinical features of fatigability and muscle weakness, but age of onset, presenting symptoms, and response to treatment vary depending on the molecular mechanism that results from the underlying genetic defect. The term CMS is misleading since not all CMS are congenital. See, Finsterer (2019) Orphanet JRare Dis. 14: 57 for a review.

Facioscapulohumeral muscular dystrophy (FSHD) type 1 is an autosomal dominant condition caused by mutations in DUX4. FSHD typically presents before age 20 years with weakness of the facial muscles and the stabilizers of the scapula or the dorsiflexors of the foot. There is extreme clinical variability. In some cases, Congenital facial weakness may be present. In FSHD, the muscle weakness is slowly progressive and approximately 20% of affected individuals eventually require a wheelchair. Life expectancy is not shortened. The incidence is approximately 4 individuals affected per 100,000 people.

Exemplary adipose tissue disorders include a metabolic disorder, e.g. metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

As used herein, a “metabolic disorder” refers to any disease or disorder that disrupts normal metabolism, the process of converting food to energy on a cellular level. Metabolic diseases affect the ability of the cell to perform critical biochemical reactions that involve the processing or transport of proteins (amino acids), carbohydrates (sugars and starches), or lipids (fatty acids). Non-limiting examples of metabolic diseases include disorders of carbohydrates, e.g., diabetes, type I diabetes, type II diabetes, galactosemia, hereditary fructose intolerance, fructose 1,6-diphosphatase deficiency, glycogen storage disorders, congenital disorders of glycosylation, insulin resistance, insulin insufficiency, hyperinsulinemia, impaired glucose tolerance (IGT), abnormal glycogen metabolism; disorders of amino acid metabolism, e.g., maple syrup urine disease (MSUD), or homocystinuria; disorder of organic acid metabolism, e.g., methylmalonic aciduria, 3-methylglutaconic aciduria-Barth syndrome, glutaric aciduria or 2-hydroxyglutaric aciduria—D and L forms; disorders of fatty acid beta-oxidation, e.g., medium-chain acyl-CoA dehydrogenase deficiency (MCAD), long-chain 3-hydroxyacyl-CoA dehydrogenase deficiency (LCHAD), very-long-chain acyl-CoA dehydrogenase deficiency (VLCAD); disorders of lipid metabolism, e.g., GM1 Gangliosidosis, Tay-Sachs Disease, Sandhoff Disease, Fabry Disease, Gaucher Disease, Niemann-Pick Disease, Krabbe Disease, Mucolipidoses, or Mucopolysaccharidoses; mitochondrial disorders, e.g., mitochondrial cardiomyopathies; Leigh disease; mitochondrial encephalopathy, lactic acidosis, and stroke-like episodes (MELAS); myoclonic epilepsy with ragged-red fibers (MERRF); neuropathy, ataxia, and retinitis pigmentosa (NARP); Barth syndrome; peroxisomal disorders, e.g., Zellweger Syndrome (cerebrohepatorenal syndrome), X-Linked Adrenoleukodystrophy, Refsum Disease, genetic obesity, Laron syndrome; Growth hormone insensitivity syndrome; Pituitary dwarfism II, adiponectin deficiency, CD36 deficiency; Platelet glycoprotein IV deficiency, Hyperlipoproteinemia, type I, Acatalasemia; Takahara disease, Glycogen storage disease type IV; Andersen disease, Acute alcohol sensitivity, Early childhood-onset progressive leukodystrophy, Secondary hyperammonemia, Glycogen storage disease of heart, 3-Methylcrotonylglycinuria; 3-Methylcrotonyl-CoA carboxylase deficiency, Leprechaunism; Donohue syndrome, Insulin-resistant diabetes mellitus with acanthosis nigricans; Type A insulin resistance, Pyruvate dehydrogenase complex deficiency, Pyruvate dehydrogenase E3-binding protein deficiency; and Lacticacidemia due to PDX1 deficiency.

In one embodiment, a metabolic disorder is metabolic syndrome. The term “metabolic syndrome, as used herein, is disorder that includes a clustering of components that reflect over nutrition, sedentary lifestyles, genetic factors, increasing age, and resultant excess adiposity. Metabolic syndrome includes the clustering of abdominal obesity, insulin resistance, dyslipidemia, and elevated blood pressure and is associated with other comorbidities including the prothrombotic state, proinflammatory state, nonalcoholic fatty liver disease, and reproductive disorders. The prevalence of the metabolic syndrome has increased to epidemic proportions not only in the United States and the remainder of the urbanized world but also in developing nations. Metabolic syndrome is associated with an approximate doubling of cardiovascular disease risk and a 5-fold increased risk for incident type 2 diabetes mellitus.

Abdominal adiposity (e.g., a large waist circumference (high waist-to-hip ratio)), high blood pressure, insulin resistance and dislipidemia are central to metabolic syndrome and its individual components (e.g., central obesity, fasting blood glucose (FBG)/pre-diabetes/diabetes, hypercholesterolemia, hypertriglyceridemia, and hypertension).

In one embodiment, a metabolic disorder is a disorder of carbohydrates. In one embodiment, the disorder of carbohydrates is diabetes.

As used herein, the term “diabetes” refers to a group of metabolic disorders characterized by high blood sugar (glucose) levels which result from defects in insulin secretion or action, or both. There are two most common types of diabetes, namely type 1 diabetes and type 2 diabetes, which both result from the body's inability to regulate insulin. Insulin is a hormone released by the pancreas in response to increased levels of blood sugar (glucose) in the blood.

The term “type I diabetes,” as used herein, refers to a chronic disease that occurs when the pancreas produces too little insulin to regulate blood sugar levels appropriately. Type I diabetes is also referred to as insulin-dependent diabetes mellitus, IDDM, and juvenile onset diabetes. People with type I diabetes (insulin-dependent diabetes) produce little or no insulin at all. Although about 6 percent of the United States population has some form of diabetes, only about 10 percent of all diabetics have type I disorder. Most people who have type I diabetes developed the disorder before age 30. Type 1 diabetes represents the result of a progressive autoimmune destruction of the pancreatic β-cells with subsequent insulin deficiency. More than 90 percent of the insulin-producing cells (beta cells) of the pancreas are permanently destroyed. The resulting insulin deficiency is severe, and to survive, a person with type I diabetes must regularly inject insulin.

In type II diabetes (also referred to as noninsulin-dependent diabetes mellitus, NDDM), the pancreas continues to manufacture insulin, sometimes even at higher than normal levels. However, the body develops resistance to its effects, resulting in a relative insulin deficiency. Type II diabetes may occur in children and adolescents but usually begins after age 30 and becomes progressively more common with age: about 15 percent of people over age 70 have type II diabetes. Obesity is a risk factor for type II diabetes, and 80 to 90 percent of the people with this disorder are obese.

In some embodiments, diabetes includes pre-diabetes. “Pre-diabetes” refers to one or more early diabetic conditions including impaired glucose utilization, abnormal or impaired fasting glucose levels, impaired glucose tolerance, impaired insulin sensitivity and insulin resistance. Prediabetes is a major risk factor for the development of type 2 diabetes mellitus, cardiovascular disease and mortality. Much focus has been given to developing therapeutic interventions that prevent the development of type 2 diabetes by effectively treating prediabetes.

Diabetes can be diagnosed by the administration of a glucose tolerance test. Clinically, diabetes is often divided into several basic categories. Primary examples of these categories include, autoimmune diabetes mellitus, non-insulin-dependent diabetes mellitus (type 1 NDDM), insulin-dependent diabetes mellitus (type 2 IDDM), non-autoimmune diabetes mellitus, non-insulin-dependent diabetes mellitus (type 2 NIDDM), and maturity-onset diabetes of the young (MODY). A further category, often referred to as secondary, refers to diabetes brought about by some identifiable condition which causes or allows a diabetic syndrome to develop. Examples of secondary categories include, diabetes caused by pancreatic disease, hormonal abnormalities, drug- or chemical-induced diabetes, diabetes caused by insulin receptor abnormalities, diabetes associated with genetic syndromes, and diabetes of other causes. (see e.g., Harrison's (1996) 14th ed., New York, McGraw-Hill).

In one embodiment, a metabolic disorder is a lipid metabolism disorder. As used herein, a “lipid metabolism disorder” or “disorder of lipid metabolism” refers to any disorder associated with or caused by a disturbance in lipid metabolism. This term also includes any disorder, disease or condition that can lead to hyperlipidemia, or condition characterized by abnormal elevation of levels of any or all lipids and/or lipoproteins in the blood. This term refers to an inherited disorder, such as familial hypertriglyceridemia, familial partial lipodystrophy type 1 (FPLD1), or an induced or acquired disorder, such as a disorder induced or acquired as a result of a disease, disorder or condition (e.g., renal failure), a diet, or intake of certain drugs (e.g., as a result of highly active antiretroviral therapy (HAART) used for treating, e.g., AIDS or HIV).

Additional examples of disorders of lipid metabolism include, but are not limited to, atherosclerosis, dyslipidemia, hypertriglyceridemia (including drug-induced hypertriglyceridemia, diuretic-induced hypertriglyceridemia, alcohol-induced hypertriglyceridemia, β-adrenergic blocking agent-induced hypertriglyceridemia, estrogen-induced hypertriglyceridemia, glucocorticoid-induced hypertriglyceridemia, retinoid-induced hypertriglyceridemia, cimetidine-induced hypertriglyceridemia, and familial hypertriglyceridemia), acute pancreatitis associated with hypertriglyceridemia, chylomicron syndrome, familial chylomicronemia, Apo-E deficiency or resistance, LPL deficiency or hypoactivity, hyperlipidemia (including familial combined hyperlipidemia), hypercholesterolemia, gout associated with hypercholesterolemia, xanthomatosis (subcutaneous cholesterol deposits), hyperlipidemia with heterogeneous LPL deficiency, hyperlipidemia with high LDL and heterogeneous LPL deficiency, fatty liver disease, or non-alcoholic stetohepatitis (NASH).

Cardiovascular diseases are also considered “metabolic disorders”, as defined herein. These diseases may include coronary artery disease (also called ischemic heart disease), hypertension, inflammation associated with coronary artery disease, restenosis, peripheral vascular diseases, and stroke.

Disorders related to body weight are also considered “metabolic disorders”, as defined herein. Such disorders may include obesity, hypo-metabolic states, hypothyroidism, uremia, and other conditions associated with weight gain (including rapid weight gain), weight loss, maintenance of weight loss, or risk of weight regain following weight loss.

Blood sugar disorders are further considered “metabolic disorders”, as defined herein. Such disorders may include diabetes, hypertension, and polycystic ovarian syndrome related to insulin resistance. Other exemplary disorders of metabolic disorders may also include renal transplantation, nephrotic syndrome, Cushing's syndrome, acromegaly, systemic lupus erythematosus, dysglobulinemia, lipodystrophy, glycogenosis type I, and Addison's disease.

In one embodiment, an adipose-tissue-associated disorder is primary hypertension. “Primary hypertension” is a result of environmental or genetic causes (e.g., a result of no obvious underlying medical cause).

In one embodiment, an adipose-tissue-associated disorder is secondary hypertension. “Secondary hypertension” has an identifiable underlying disorder which can be of multiple etiologies, including renal, vascular, and endocrine causes, e.g., renal parenchymal disease (e.g., polycystic kidneys, glomerular or interstitial disease), renal vascular disease (e.g., renal artery stenosis, fibromuscular dysplasia), endocrine disorders (e.g., adreno cortico steroid or mineralocorticoid excess, pheochromocytoma, hyperthyroidism or hypothyroidism, growth hormone excess, hyperparathyroidism), coarctation of the aorta, or oral contraceptive use.

In one embodiment, an adipose-tissue-associated disorder is resistant hypertension. “Resistant hypertension” is blood pressure that remains above goal (e.g., above 130 mm Hg systolic or above 90 diastolic) in spite of concurrent use of three antihypertensive agents of different classes, one of which is a thiazide diuretic. Subjects whose blood pressure is controlled with four or more medications are also considered to have resistant hypertension.

Additional diseases or conditions related to metabolic disorders that would be apparent to the skilled artisan and are within the scope of this disclosure.

“Therapeutically effective amount,” as used herein, is intended to include the amount of an RNAi agent that, when administered to a subject having a target gene-associated disease, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of a RNAi agent that, when administered to a subject having a target gene-associated disorder, e.g., gout or diabetes, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylacticaly effective amount” also includes an amount of a RNAi agent that produces some desired local or systemic effect at a reasonable benefit/risk ratio applicable to any treatment. A RNAi agent employed in the methods of the present disclosure may be administered in a sufficient amount to produce a reasonable benefit/risk ratio applicable to such treatment.

The phrase “pharmaceutically acceptable” is employed herein to refer to those compounds, materials (including salts), compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations. Pharmaceutically acceptable carriers for pulmonary delivery are known in the art and will vary depending on the desired location for deposition of the agent, e.g., upper or lower respiratory system, and the type of device to be used for delivery, e.g., sprayer, nebulizer, dry powder inhaler.

The term “sample,” as used herein, includes a collection of similar fluids, cells, or tissues isolated from a subject, as well as fluids, cells, or tissues present within a subject. Examples of biological fluids include blood, serum and serosal fluids, plasma, bronchial fluids, sputum, cerebrospinal fluid, ocular fluids, lymph, urine, saliva, sputum, and the like. Tissue samples may include samples from tissues, organs or localized regions. For example, samples may be derived from particular organs, parts of organs, or fluids or cells within those organs.

### II. RNAi Agents of the Invention

Described herein are RNAi agents comprising one or more C22 hydrocarbon chains, e.g., saturated or unsaturated, conjugated to one or more internal positions on at least one strand which inhibit the expression of a target gene in muscle tissue or an adipose tissue. In one embodiment, the RNAi agent includes double stranded ribonucleic acid (dsRNA) molecules for inhibiting the expression of a target gene in a cell, such as a cell within a subject, e.g., a mammal, such as a human, e.g., a subject having a target gene-associated disorder, e.g., a muscle tissue disease or an adipose tissue disease, or a subject at risk of a target gene-associated disease, e.g., a muscle tissue disease or an adipose tissue disease.

The dsRNA includes an antisense strand having a region of complementarity which is complementary to at least a part of a target RNA, e.g., an mRNA formed in the expression of a target gene. The region of complementarity is about 15-30 nucleotides or less in length. Upon contact with a cell expressing the target gene, the RNAi agent inhibits the expression of the target gene (e.g., a human gene, a primate gene, a non-primate gene) by at least 50% as assayed by, for example, a PCR or branched DNA (bDNA)-based method, or by a protein-based method, such as by immunofluorescence analysis, using, for example, western blotting, flowcytometric techniques, or histology based method such as immunohistochemistry or in situ hybridization. In certain embodiments, inhibition of expression is by at least 50% as assayed by the Dual-Glo lucifierase assay in Example 1 where the siRNA is at a 10 nM concentration.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. For example, the target sequence can be derived from the sequence of an mRNA formed during the expression of a target gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24,20-23, 20-22, 20-21, 21-25, 21-24, 21-23, 21-22, 22-25, 22-24, 22-23, 23-25, 23-24 or 24-25 base pairs in length, for example, 19-21 basepairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the dsRNA is 15 to 23 nucleotides in length, or 25 to 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 15 to 36 base pairs, e.g., 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs, for example, 19-21 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, a RNAi agent useful to target gene expression is not generated in the target cell by cleavage of a larger dsRNA.

A dsRNA as described herein can further include one or more single-stranded nucleotide overhangs e.g., 1, 2, 3, or 4 nucleotides. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA. In certain embodiments, longer, extended overhangs are possible.

A dsRNA can be synthesized by standard methods known in the art as further discussed below, e.g., by use of an automated DNA synthesizer, such as are commercially available from, for example, Biosearch, Applied Biosystems, Inc.

iRNA compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the invention can be prepared using solution-phase or solid-phase organic synthesis or both.

An iRNA can be produced, e.g., in bulk, by a variety of methods. Exemplary methods include: organic synthesis and RNA cleavage, e.g., in vitro cleavage.

An iRNA can be made by separately synthesizing a single stranded RNA molecule, or each respective strand of a double-stranded RNA molecule, after which the component strands can then be annealed.

A large machine, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete iRNA species. The complementary of the species to a target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

In one embodiment, dsRNA is cleaved in vitro into siRNAs, for example, using a Dicer or comparable RNAse III-based activity. For example, the dsRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9 and Hammond Science 2001 Aug. 10; 293(5532):1146-50.

dsRNA cleavage generally produces a plurality of siRNA species, each being a particular 21 to 23 nucleotide fragment of a source dsRNA molecule. For example, siRNAs that include sequences complementary to overlapping regions and adjacent regions of a source dsiRNA molecule may be present.

Regardless of the method of synthesis, the dsRNA preparation can be prepared in a solution (e.g., an aqueous or organic solution) that is appropriate for formulation. For example, the dsRNA preparation can be precipitated and redissolved in pure double-distilled water, and lyophilized. The dried dsRNA can then be resuspended in a solution appropriate for the intended formulation process.

In one aspect, a dsRNA of the disclosure includes at least two nucleotide sequences, a sense sequence and an antisense sequence.

In one embodiment, the dsRNA of the disclosure targets the ADRB1 gene. The sense strand sequence for ADRB1 may be selected from the group of sequences provided in any one of Tables 2-5, 7B, and 7C, and the corresponding nucleotide sequence of the antisense strand of the sense strand may be selected from the group of sequences of any one of Tables 2-5, 7B, and 7C. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand (passenger strand) in any one of Tables 2-5, 7B, and 7C, and the second oligonucleotide is described as the corresponding antisense strand (guide strand) of the sense strand in any one of Tables 2-5, 7B, and 7C for ADRB1.

In one embodiment, the dsRNA of the disclosure targets the Leptin (LEP) gene. The sense strand sequence for LEP may be selected from the group of sequences provided in any one of Tables 9-16, and the corresponding nucleotide sequence of the antisense strand of the sense strand may be selected from the group of sequences of any one of Tables 9-16. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand (passenger strand) in any one of Tables 9-16, and the second oligonucleotide is described as the corresponding antisense strand (guide strand) of the sense strand in any one of Tables 9-16 for LEP.

In one embodiment, the dsRNA of the disclosure targets the PLN gene. The sense strand sequence for PLN may be selected from the group of sequences provided in any one of Tables 19-22, and the corresponding nucleotide sequence of the antisense strand of the sense strand may be selected from the group of sequences of any one of Tables 19-22. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand (passenger strand) in any one of Tables 19-22, and the second oligonucleotide is described as the corresponding antisense strand (guide strand) of the sense strand in any one of Tables 19-22 for PLN.

In one embodiment, the dsRNA of the disclosure targets the CAMK2D gene. The sense strand sequence for CAMK2D may be selected from the group of sequences provided in any one of Tables 23-26, and the corresponding nucleotide sequence of the antisense strand of the sense strand may be selected from the group of sequences of any one of Tables 23-26. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand (passenger strand) in any one of Tables 23-26, and the second oligonucleotide is described as the corresponding antisense strand (guide strand) of the sense strand in any one of Tables 23-26 for CAMK2D.

In one embodiment, the substantially complementary sequences of the dsRNA are contained on separate oligonucleotides. In another embodiment, the substantially complementary sequences of the dsRNA are contained on a single oligonucleotide.

It will be understood that, although the sequences provided herein are described as modified or conjugated sequences, the RNA of the RNAi agent of the disclosure e.g., a dsRNA of the disclosure, may comprise any one of the sequences set forth in any one of Tables 2-5, 7B, 7C, 9-16, and 19-26 that is un-modified, un-conjugated, or modified or conjugated differently than described therein. One or more lipophilic ligands or one or more GalNAc ligands can be included in any of the positions of the RNAi agents provided in the instant application.

The skilled person is well aware that dsRNAs having a duplex structure of about 20 to 23 base pairs, e.g., 21, base pairs have been hailed as particularly effective in inducing RNA interference (Elbashir et al., (2001) EMBO J., 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of a target gene by not more than 10, 15, 20, 25, or 30% inhibition from a dsRNA comprising the full sequence using the in vitro assay with Cos 7 and a 10 nM concentration of the RNA agent and the PCR assay as provided in the examples herein, are contemplated to be within the scope of the present disclosure.

In addition, the RNAs described herein identify a site(s) in a target gene transcript that is susceptible to RISC-mediated cleavage. As such, the present disclosure further features RNAi agents that target within this site(s). As used herein, a RNAi agent is said to target within a particular site of an RNA transcript if the RNAi agent promotes cleavage of the transcript anywhere within that particular site. Such a RNAi agent will generally include at least about 15 contiguous nucleotides, such as at least 19 nucleotides, from one of the sequences provided herein coupled to additional nucleotide sequences taken from the region contiguous to the selected sequence in a target gene.

An RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a target gene generally does not contain any mismatch within the central 13 nucleotides. The methods described herein or methods known in the art can be used to determine whether an RNAi agent containing a mismatch to a target sequence is effective in inhibiting the expression of a target gene. Consideration of the efficacy of RNAi agents with mismatches in inhibiting expression of a target gene is important, especially if the particular region of complementarity in a target gene is known to mutate.

### III. C₂₂ Hydrocarbon Chains

As described herein, conjugating a C₂₂ hydrocarbon chain, e.g., saturated or unsaturated, to one or more internal position(s) of the dsRNA agent increases lipophilicity of the dsRNA agent and provides optimal hydrophobicity for the enhanced in vivo delivery of dsRNA to muscle tissue, e.g., skeletal muscle tissue or cardiac muscle tissue, or adipose tissue.

One way to characterize lipophilicity is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J Chem. Inf Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a C₂₂ hydrocarbon chain can increase or decrease the partition coefficient (e.g., log K_{ow}) value of the C₂₂ hydrocarbon chain.

Alternatively, the hydrophobicity of the dsRNA agent, conjugated to one or more C₂₂ hydrocarbon chains, can be measured by its protein binding characteristics. For instance, the unbound fraction in the plasma protein binding assay of the dsRNA agent can be determined to positively correlate to the relative hydrophobicity of the dsRNA agent, which can positively correlate to the silencing activity of the dsRNA agent.

In one embodiment, the plasma protein binding assay determined is an electrophoretic mobility shift assay (EMSA) using human serum albumin protein. The hydrophobicity of the dsRNA agent, measured by fraction of unbound dsRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

In certain embodiments, the one or more C₂₂ hydrocarbon chains is an aliphatic, alicyclic, or polyalicyclic compound is an aliphatic, cyclic such as alicyclic, or polycyclic such as polyalicyclic compound. The hydrocarbon chain may comprise various substituents and/or one or more heteroatoms, such as an oxygen or nitrogen atom.

The one or more C₂₂ hydrocarbon chains may be attached to the iRNA agent by any method known in the art, including via a functional grouping already present in the lipophilic moiety or introduced into the iRNA agent, such as a hydroxy group (e.g., —CO—CH₂—OH). The functional groups already present in the C₂₂ hydrocarbon chain or introduced into the dsRNA agent include, but are not limited to, hydroxyl, amine, carboxylic acid, sulfonate, phosphate, thiol, azide, and alkyne.

Conjugation of the dsRNA agent and the C₂₂ hydrocarbon chain may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the C₂₂ hydrocarbon chain is conjugated to the dsRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In one embodiment, the one or more C₂₂ hydrocarbon chains is a C₂₂ acid, e.g., the C₂₂ acid is selected from the group consisting of docosanoic acid, 6-octyltetradecanoic acid, 10-hexylhexadecanoic acid, all-cis-7,10,13,16,19-docosapentaenoic acid, all-cis-4,7,10,13,16,19-docosahexaenoic acid, all-cis-13,16-docosadienoic acid, all-cis-7,10,13,16-docosatetraenoic acid, all-cis-4,7,10,13,16-docosapentaenoic acid, and cis-13-docosenoic acid.

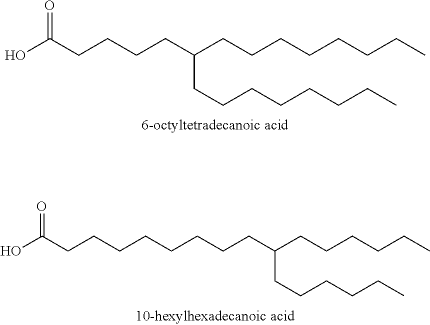 [SMILES: CCCCCCCCC(CCCCCCCC)CCCCC(=O)O ; CCCCCCC(CCCCCC)CCCCCCCCC(=O)O]

In one embodiment, the one or more C₂₂ hydrocarbon chains is a C₂₂ alcohol, e.g. the C₂₂ alcohol is selected from the group consisting of 1-docosanol, 6-octyltetradecan-1-ol, 10-hexylhexadecan-1-ol, cis-13-docosen-1-ol, docosan-9-ol, docosan-2-ol, docosan-10-ol, docosan-11-ol, and cis-4,7,10,13,16,19-docosahexanol.

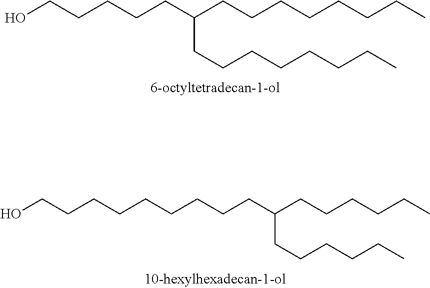 [SMILES: CCCCCCCCC(CCCCCO)CCCCCCCC ; CCCCCCC(CCCCCC)CCCCCCCCCO]

In one embodiment the one or more C₂₂ hydrocarbon chains is not cis-4,7,10,13,16,19-docosahexanoic acid. In one embodiment, the one or more C₂₂ hydrocarbon chains is not cis-4,7,10,13,16,19-docosahexanol. In one embodiment, the one or more C₂₂ hydrocarbon chains is not cis-4,7,10,13,16,19-docosahexanoic acid and is not cis-4,7,10,13,16,19-docosahexanol.

In one embodiment, the one or more C₂₂ hydrocarbon chains is a C₂₂ amide, e.g., the C₂₂ amide is selected from the group consisting of (E)-Docos-4-enamide, (E)-Docos-5-enamide, (Z)-Docos-9-enamide, (E)-Docos-11-enamide, 12-Docosenamide, (Z)-Docos-13-enamide, (Z)—N-Hydroxy-13-docoseneamide, (E)-Docos-14-enamide, 6-cis-Docosenamide, 14-Docosenamide Docos-11-enamide, (4E, 13E)-Docosa-4,13-dienamide, and (5E, 13E)-Docosa-5,13-dienamide.

In certain embodiments, more than one C₂₂ hydrocarbon chains can be incorporated into the double-strand iRNA agent, particularly when the C₂₂ hydrocarbon chains has a low lipophilicity or hydrophobicity. In one embodiment, two or more C₂₂ hydrocarbon chains are incorporated into the same strand of the double-strand iRNA agent. In one embodiment, each strand of the double-strand iRNA agent has one or more C₂₂ hydrocarbon chains incorporated. In one embodiment, two or more C₂₂ hydrocarbon chains are incorporated into the same position (i.e., the same nucleobase, same sugar moiety, or same internucleosidic linkage) of the double-stranded iRNA agent. This can be achieved by, e.g., conjugating the two or more saturated or unsaturated C₂₂ hydrocarbon chains via a carrier, and/or conjugating the two or more C₂₂ hydrocarbon chains via a branched linker, and/or conjugating the two or more C₂₂ hydrocarbon chains via one or more linkers, with one or more linkers linking the C₂₂ hydrocarbon chains consecutively.

The one or more C₂₂ hydrocarbon chains may be conjugated to the iRNA agent via a direct attachment to the ribosugar of the iRNA agent. Alternatively, the one or more C₂₂ hydrocarbon chains may be conjugated to the double-strand iRNA agent via a linker or a carrier.

In certain embodiments, the one or more C₂₂ hydrocarbon chains may be conjugated to the iRNA agent via one or more linkers (tethers).

In one embodiment, the one or more C₂₂ hydrocarbon chains is conjugated to the dsRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

### A. Linkers Tethers

Linkers/Tethers are connected to the one or more C₂₂ hydrocarbon chains at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the lipophilic moiety. Non-limited examples of linkers/tethers (underlined) include TAP-(CH₂)ₙNH—; TAP-C(O)(CH₂)ₙNH—; TAP-NR″″(CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂A; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂), NH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)ₙ—C(O)O(LIGAND); TAP-(CH₂)ₙ(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)ₙC(O)ONHS; TAP-C(O)(CH₂), C(O)ONHS; or TAP-NR′″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O) OC₆F₅; or TAP-NR″″(CH₂)ₙC(O) OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K)

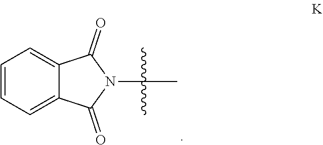 [SMILES: *N1C(=O)c2ccccc2C1=O]

at the terminal position of the linker/tether.

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### B. Cleavable Linkers/Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the rapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### C. Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### D. Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O— —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S— —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### E. Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### F. Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### G. Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### H. Biocleavable Linkers Tethers

The linkers can also includes biocleavable linkers that are nucleotide and non-nucleotide linkers or combinations thereof that connect two parts of a molecule, for example, one or both strands of two individual siRNA molecules to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, hetercyclic, and combinations thereof.

In some embodiments, at least one of the linkers (tethers) is a bio-clevable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable linkers include:

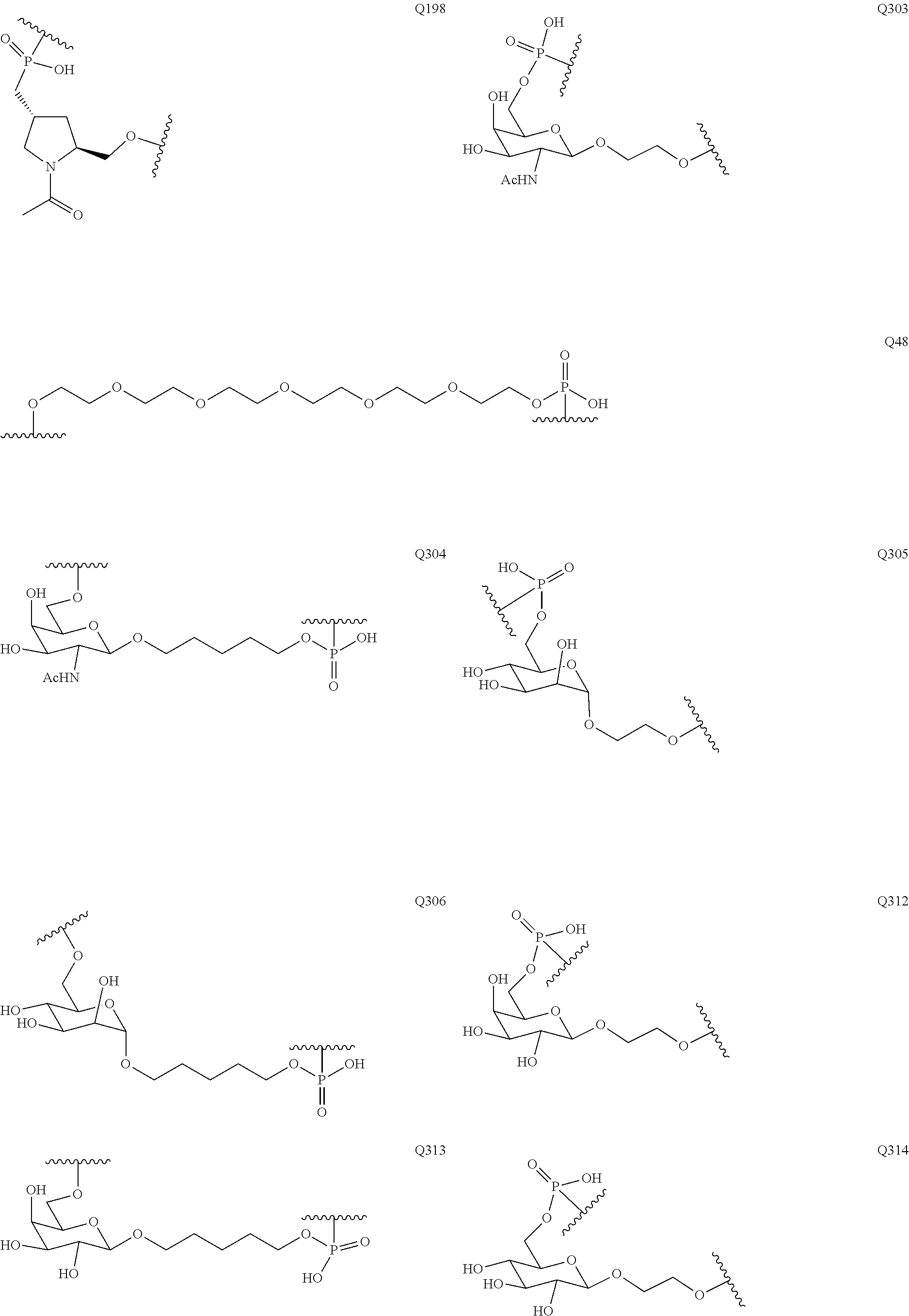

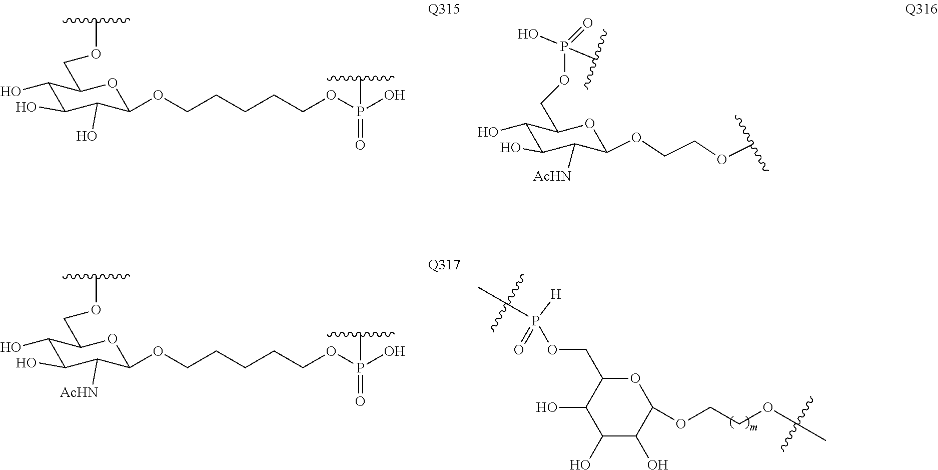

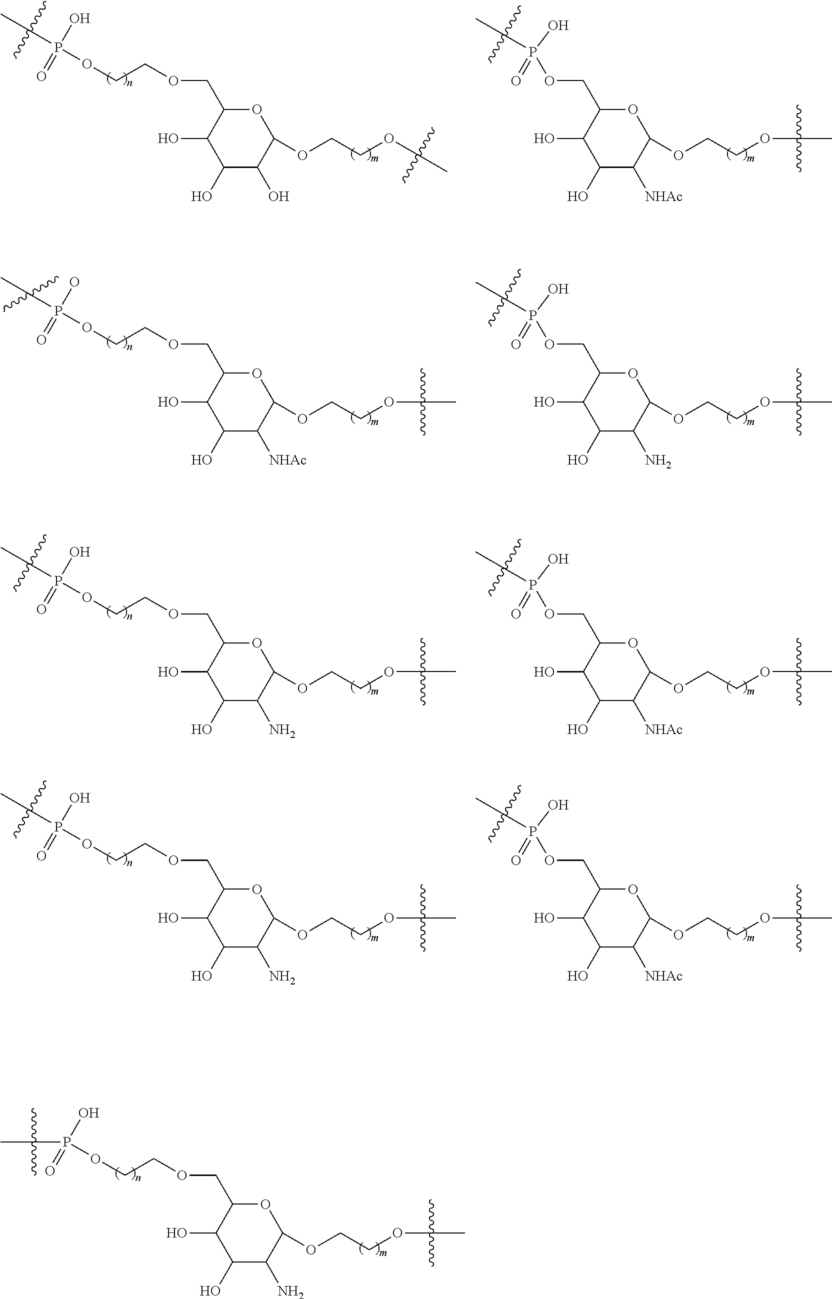

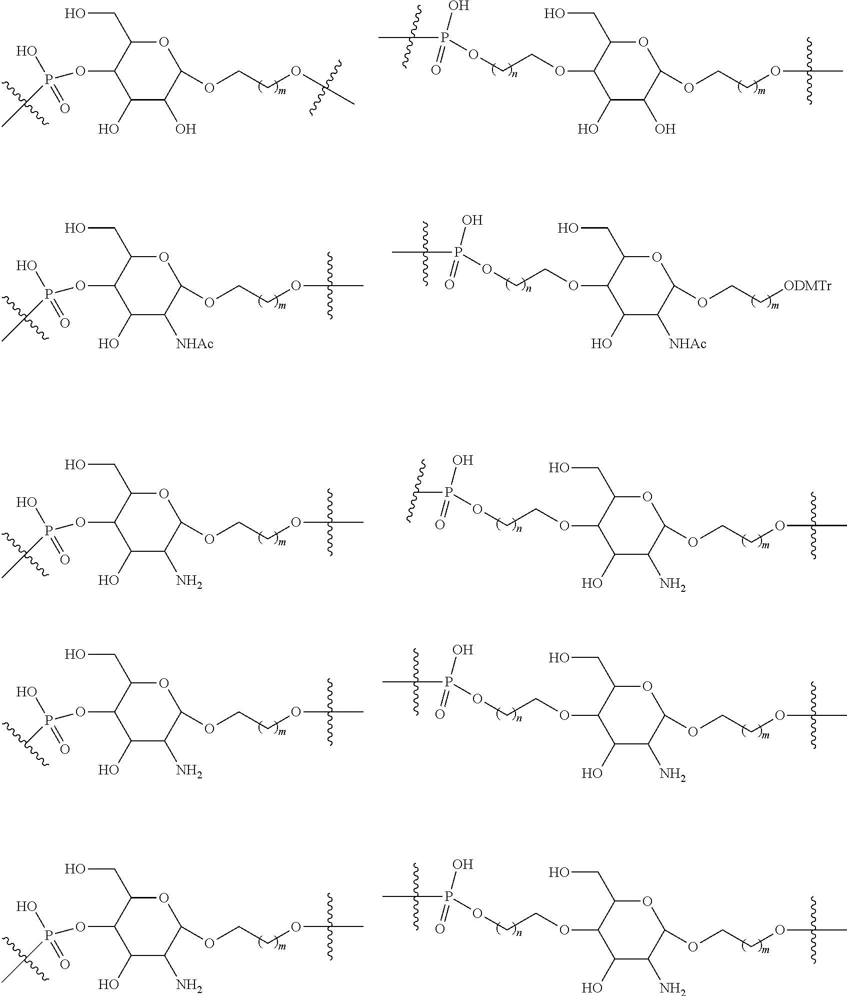

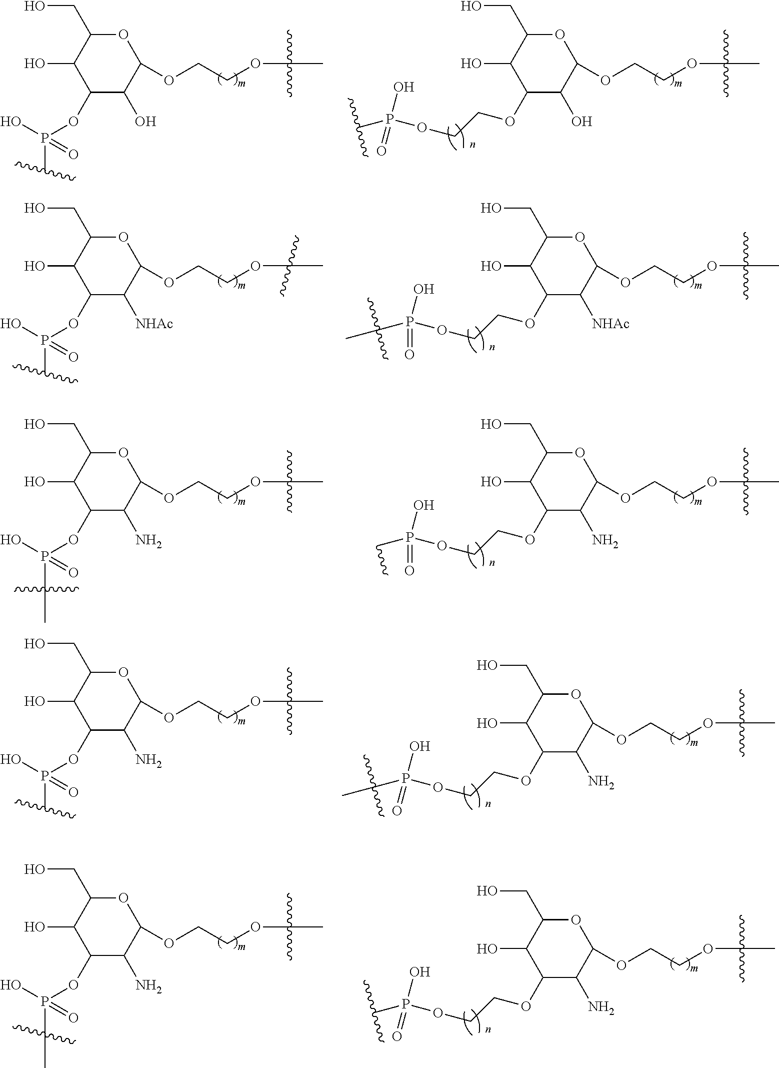

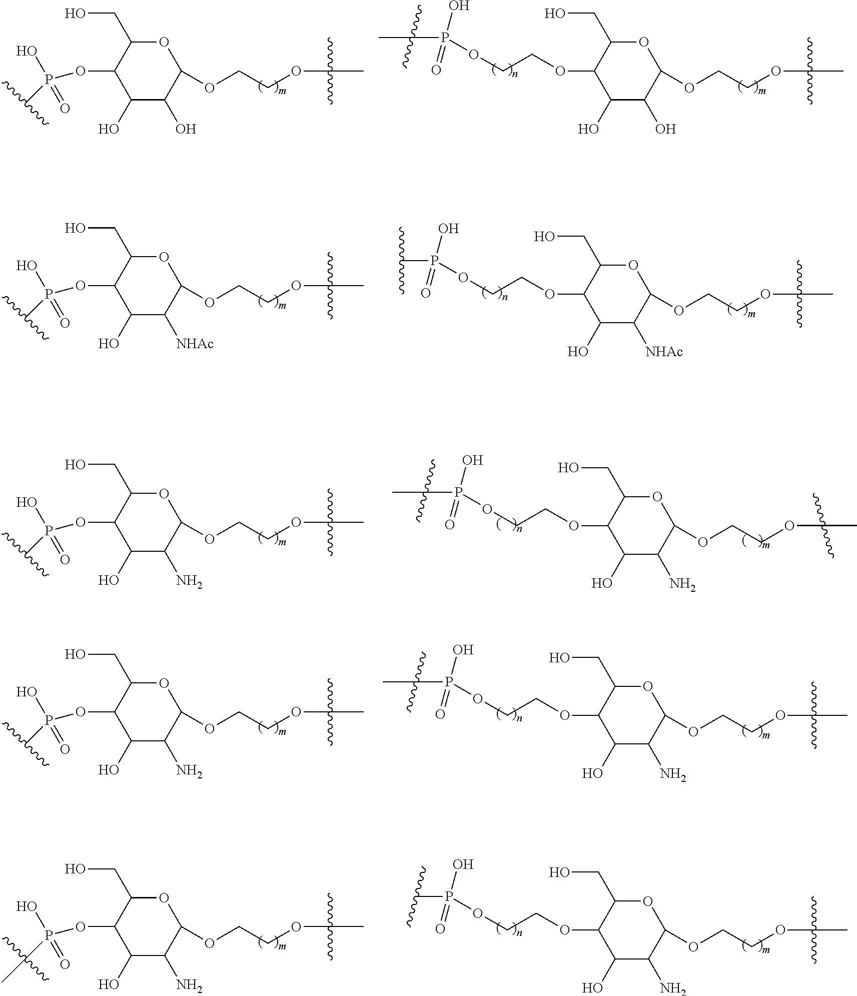

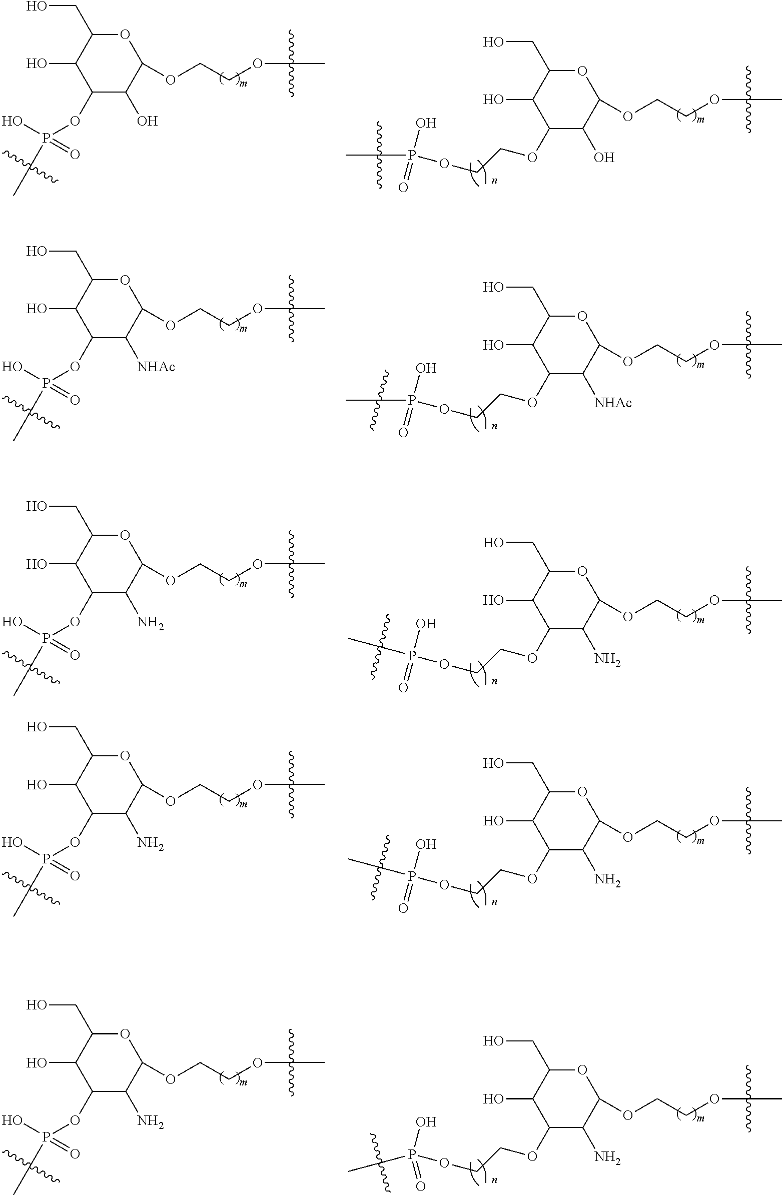

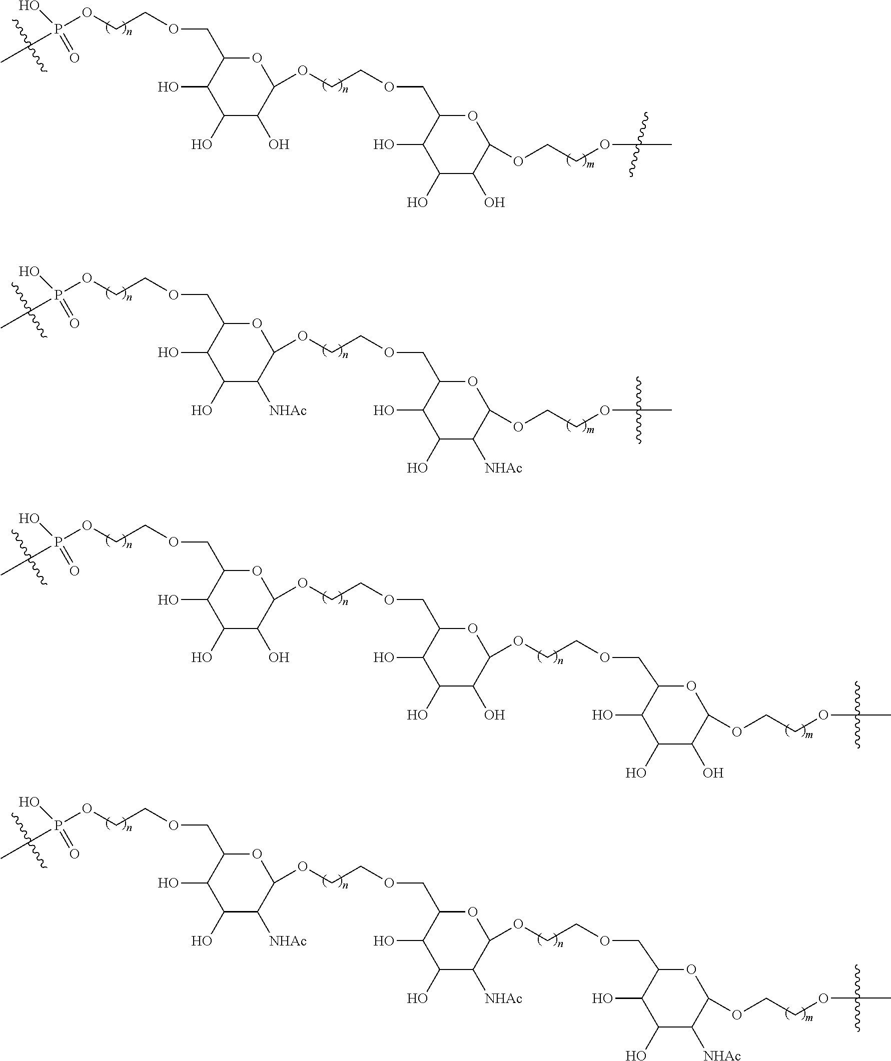

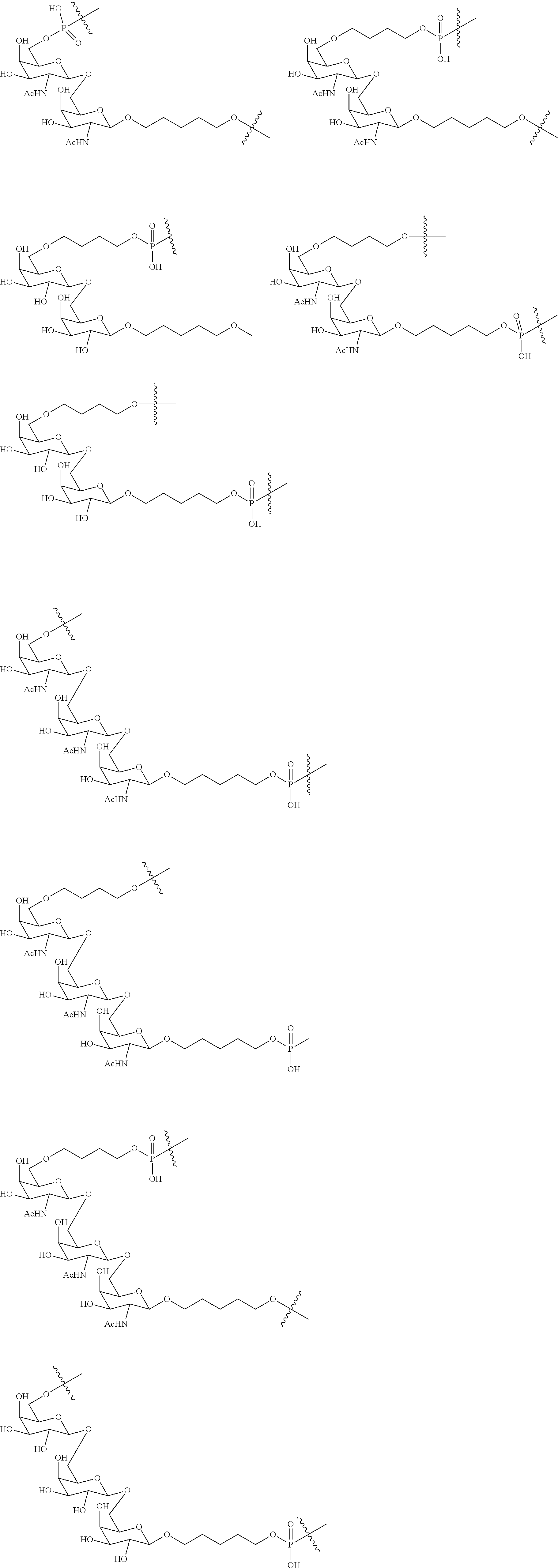

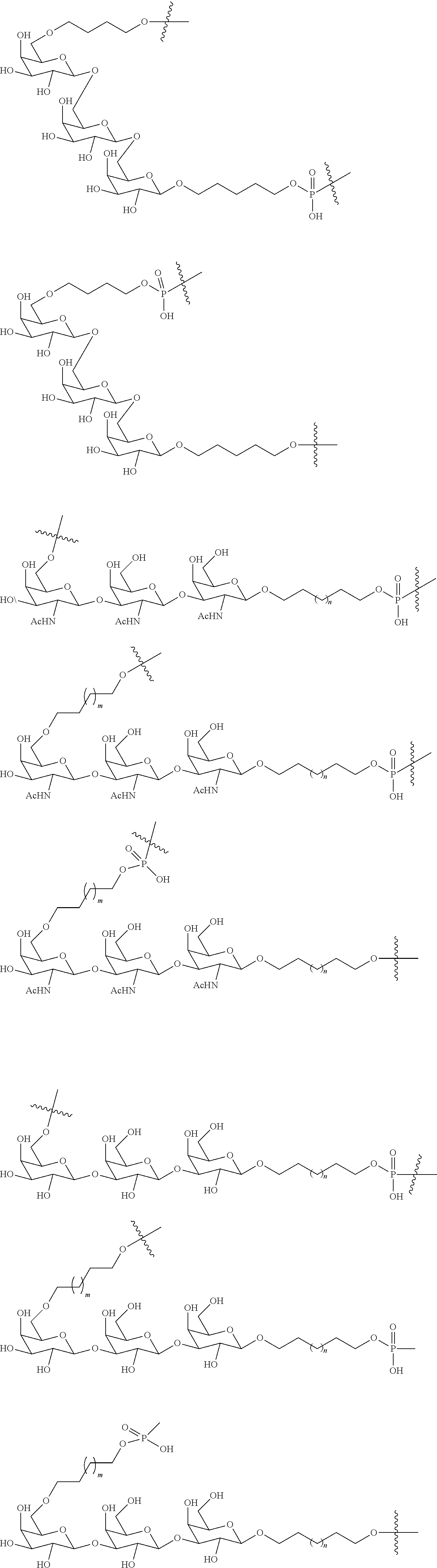

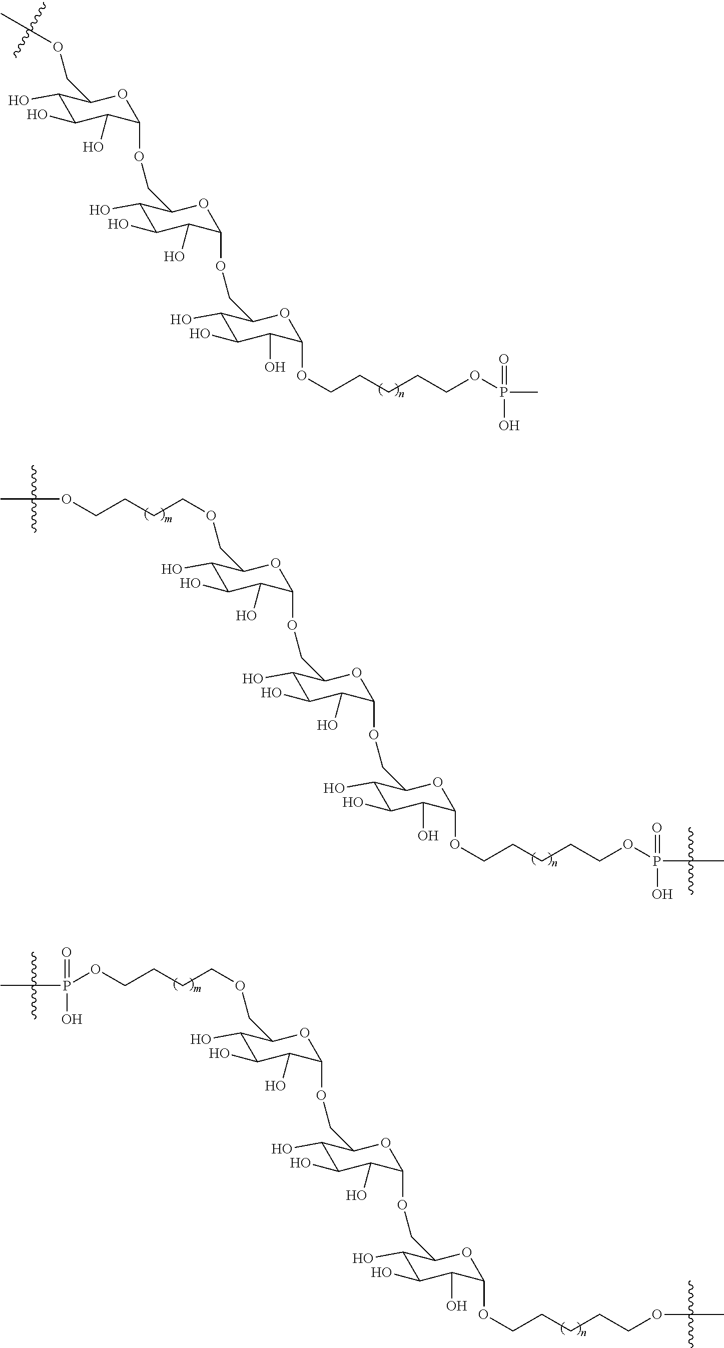

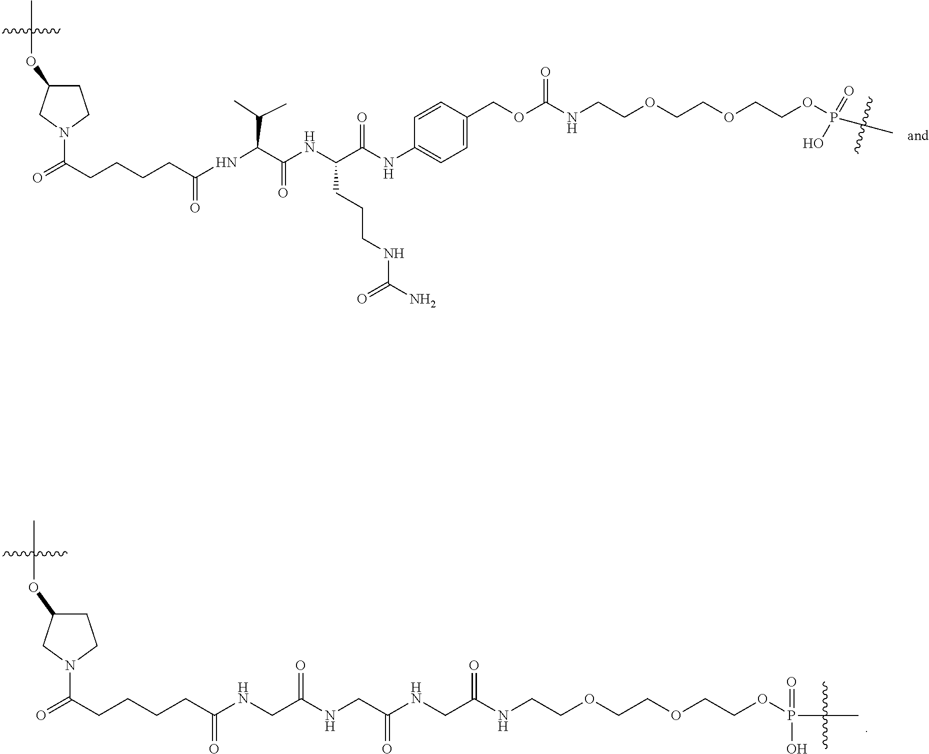 [SMILES: *O[C@H]1CCN(C(=O)CCCCC(=O)N[C@H](C(=O)N[C@@H](CCCNC(N)=O)C(=O)Nc2ccc(COC(=O)NCCOCCOCCOP(*)(=O)O)cc2)C(C)C)C1 ; *O[C@H]1CCN(C(=O)CCCCC(=O)NCC(=O)NCC(=O)NCC(=O)NCCOCCOCCOP(*)(=O)O)C1]

More discussion about the biocleavable linkers may be found in PCT application No. PCT/US18/14213, entitled “Endosomal Cleavable Linkers,” filed on Jan. 18, 2018, the entire contents of which are incorporated herein by reference.

### 1. Carriers

In certain embodiments, the one or more C₂₂ hydrocarbon chains is conjugated to the iRNA agent via a carrier that replaces one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the dsRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand (e.g., the lipophilic moiety). The one or more C₂₂ hydrocarbon chains can be directly attached to the carrier or indirectly attached to the carrier by an intervening linker/tether, as described above.

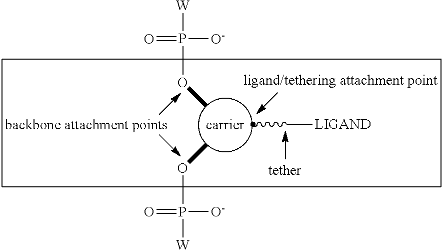

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an iRNA agent.

a. Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (in that structure preferred backbone attachment points can be chosen from R^{t} or R²; R³ or R4; or R⁹ and R¹⁰ if Y is CR⁹R¹⁰ (two positions are chosen to give two backbone attachment points, e.g., R^{t} and R⁴, or R⁴ and R⁹)). Preferred tethering attachment points include R7; R or R when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

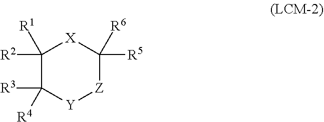

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C1-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or Si(X^{5′})(X^{5″})(X⁵″′) in which (X^{5″}), (X^{5″}), and (X⁵″′) are as described elsewhere.
R is P(O)(O⁻)H, P(OR″)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

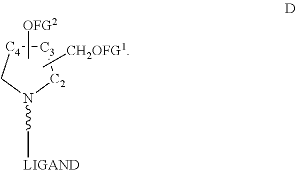

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

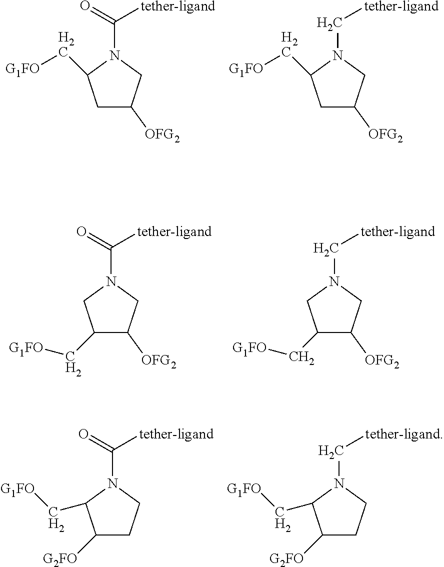

In certain embodiments, the carrier may be based on the piperidine ring system (E), e.g., X is N(CO)R⁷ or NR⁷, Y is CR⁹R¹⁰, and Z is CR¹¹R¹²

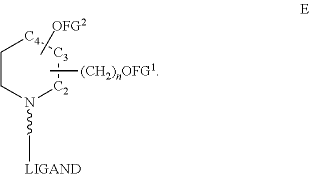

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR^{B}, and Z is CR¹¹R¹², or the morpholine ring system (G), e.g., X is N(CO)R⁷ or NR⁷, Y is O, and Z is CR¹¹R¹²

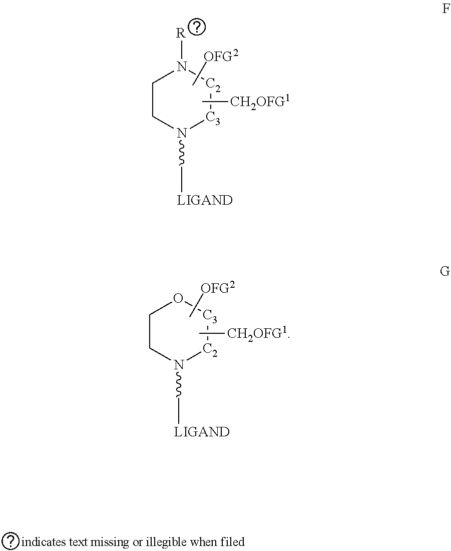

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R″′ can be, e.g., C1-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

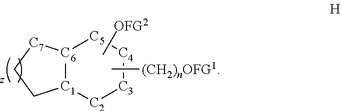

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

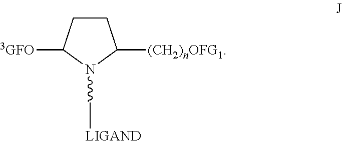

Thus, —(CH₂)ₙOFG¹ and OFG² may be cis or trans with respect to one another. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

b. Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

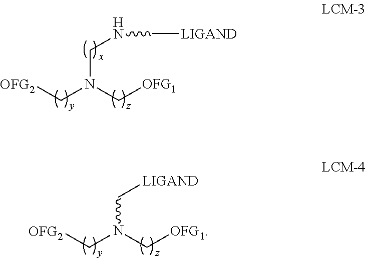

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

The one or more C₂₂ hydrocarbon chains is conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, the one or more C₂₂ hydrocarbon chains is conjugated to one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the one or more C₂₂ hydrocarbon chains is conjugated to one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, the one or more C₂₂ hydrocarbon chains is conjugated to one or more internal positions on at least one strand, except the cleavage site region of the sense strand, for instance, the one or more C₂₂ hydrocarbon chains is not conjugated to positions 9-12 counting from the 5′-end of the sense strand, for example, the one or more C₂₂ hydrocarbon chains is not conjugated to positions 9-11 counting from the 5′-end of the sense strand. Alternatively, the internal positions exclude positions 11-13 counting from the 3′-end of the sense strand.

In one embodiment, the one or more C₂₂ hydrocarbon chains is conjugated to one or more internal positions on at least one strand, which exclude the cleavage site region of the antisense strand. For instance, the internal positions exclude positions 12-14 counting from the 5′-end of the antisense strand.

In one embodiment, the one or more C₂₂ hydrocarbon chains is conjugated to one or more internal positions on at least one strand, which exclude positions 11-13 on the sense strand, counting from the 3′-end, and positions 12-14 on the antisense strand, counting from the 5′-end.

In one embodiment, the one or more C₂₂ hydrocarbon chains is conjugated to one or more of the following internal positions: positions 4-8 and 13-18 on the sense strand, and positions 6-10 and 15-18 on the antisense strand, counting from the 5′ end of each strand.

In one embodiment, the one or more C₂₂ hydrocarbon chains is conjugated to one or more of the following internal positions: positions 5, 6, 7, 15, and 17 on the sense strand, and positions 15 and 17 on the antisense strand, counting from the 5′ end of each strand.

In one embodiment, the one or more C₂₂ hydrocarbon chains is conjugated to position 6 on the sense strand, counting from the 5′ end of each strand.

In some embodiments, the one or more C₂₂ hydrocarbon chains is conjugated to a nucleobase, sugar moiety, or internucleosidic phosphate linkage of the dsRNA agent.

### IV. Additional Modifications for the RNAi Agents of the Invention

In one embodiment, the RNAi agent of the disclosure comprising one or more C₂₂ hydrocarbon chains conjugated to one or more internal positions on at least one strand, does not comprise chemical modifications known in the art and described herein, in the remaining positions of the sense and anti-sense strands.

In some embodiments, the dsRNA agents of the invention comprising one or more C₂₂ hydrocarbon chains conjugated to one or more internal positions on at least one strand, comprise at least one additional nucleic acid modification described herein. For example, at least one modification selected from the group consisting of modified internucleoside linkage, modified nucleobase, modified sugar, and any combinations thereof. Without limitations, such a modification can be present anywhere in the dsRNA agent of the invention. For example, the modification can be present in one of the RNA molecules.

Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of RNAi agents useful in the embodiments described herein include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified RNAi agent will have a phosphorus atom in its internucleoside backbone.

### A. Nucleobase Modifications

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶,N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

### B. Sugar Modifications

DsRNA agent of the inventions provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furnaosyl is connected to the 4′ position by a linker selected independently from —[C(R1)(R2)]ₙ—, —[C(R1)(R2)]ₙ—O—, —[C(R1)(R2)]ₙ—N(R1)-, —[C(R1)(R2)]ₙ—N(R1)-O—, —[C(R1R2)]ₙ—O—N(R1)-, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ, and —N(R1)-;
wherein:
x is 0, 1, or 2;
n is 1, 2, 3, or 4;
each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]ₙ—, —[C(R1)(R2)]ₙ—O—, —C(R1R2)-N(R1)-O— or —C(R1R2)-O—N(R1)-. In another embodiment, each of said linkers is, independently, 4′-CH₂-2′, 4′-(CH₂)2-2′, 4′-(CH₂)3-2′, 4′-CH₂—O-2′, 4′-(CH₂)2-O-2′, 4′-CH₂—O—N(R1)-2′ and 4′-CH₂—N(R1)-O-2′- wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂-0-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the rmal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)2-O-2′ bridge) ENA; substituted sugars, especially 2′-substituted sugars having a 2′-F, 2′-OCH₃ or a 2′-O(CH₂)2-OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)˜CH₂CH₂OR, n=1-50; “locked” nucleic acids (LNA) in which the furanose portion of the nucleoside includes a bridge connecting two carbon atoms on the furanose ring, thereby forming a bicyclic ring system; 0-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)˜CH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

DsRNA agent of the inventions disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. DsRNA agent of the inventions can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

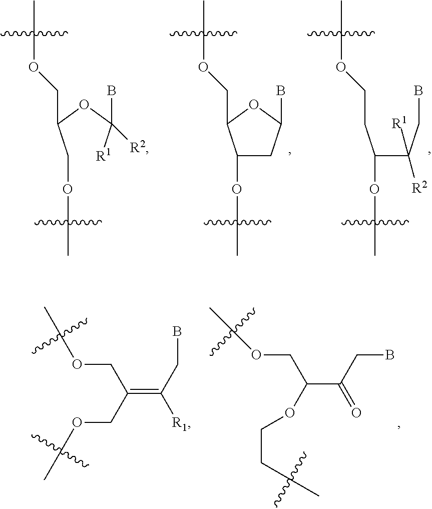

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-0-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-0-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

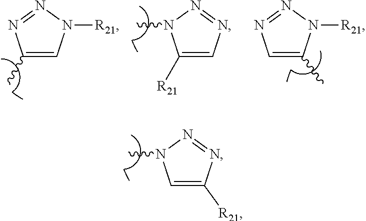

NR₂₁R₃₁, CONR₂₁R₃₁, CON(H)NR₂₁R₃₁, ONR₂₁R₃₁, CON(H)N=CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄, R₅₁, SO₂R₁₁, SOR₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the iRNA.

In certain embodiments, LNA's include bicyclic nucleoside having the formula:

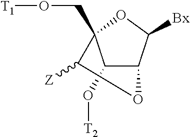

wherein:
Bₓ is a heterocyclic base moiety;
T₁ is H or a hydroxyl protecting group;
T₂ is H, a hydroxyl protecting group or a reactive phosphorus group;
Z is C1-C6 alkyl, C2-C6 alkenyl, C2-C6 alkynyl, substituted C1-C6 alkyl, substituted C2-C6 alkenyl, substituted C2-C6 alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is O, S or NJ1.

In certain such embodiments, each of the substituted groups, is, independently, mono or poly substituted with substituent groups independently selected from halogen, oxo, hydroxyl, OJ1, NJ11J2, SJ1, N3, OC(═X)J1, and NJ3C(═X)NJ1IJ2, wherein each J1, J2 and J3 is, independently, H, C1-C6 alkyl, or substituted C1-C6 alkyl and X is 0 or NJ1.

In certain embodiments, the Z group is C1-C6 alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, the Z group is C1-C6 alkyl substituted with one or more Xx, wherein each Xx is independently halo (e.g., fluoro), hydroxyl, alkoxy (e.g., CH₃O—), substituted alkoxy or azido.

In certain embodiments, the Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, the Z group is —CH₂Xx, wherein Xx is halo (e.g., fluoro), hydroxyl, alkoxy (e.g., CH₃O—) or azido.

In certain such embodiments, the Z group is in the (R)-configuration:

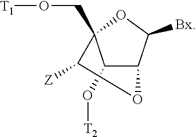

In certain such embodiments, the Z group is in the (S)-configuration:

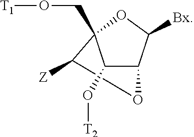

In certain embodiments, each T₁ and T₂ is a hydroxyl protecting group. A preferred list of hydroxyl protecting groups includes benzyl, benzoyl, 2,6-dichlorobenzyl, t-butyldimethylsilyl, t-butyldiphenylsilyl, mesylate, tosylate, dimethoxytrityl (DMT), 9-phenylxanthine-9-yl (Pixyl) and 9-(p-methoxyphenyl)xanthine-9-yl (MOX). In certain embodiments, T₁ is a hydroxyl protecting group selected from acetyl, benzyl, t-butyldimethylsilyl, t-butyldiphenylsilyl and dimethoxytrityl wherein a more preferred hydroxyl protecting group is T₁ is 4,4′-dimethoxytrityl.

In certain embodiments, T₂ is a reactive phosphorus group wherein preferred reactive phosphorus groups include diisopropylcyanoethoxy phosphoramidite and H-phosphonate. In certain embodiments T₁ is 4,4′-dimethoxytrityl and T₂ is diisopropylcyanoethoxy phosphoramidite.

In certain embodiments, the compounds of the invention comprise at least one monomer of the formula:

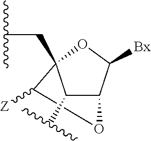

or of the formula:

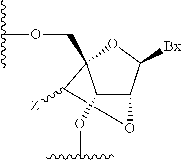

or of the formula:

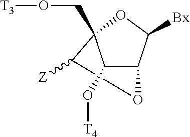

wherein
Bₓ is a heterocyclic base moiety;
T₃ is H, a hydroxyl protecting group, a linked conjugate group or an internucleoside linking group attached to a nucleoside, a nucleotide, an oligonucleoside, an oligonucleotide, a monomeric subunit or an oligomeric compound;
T₄ is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T₃ and T₄ is an internucleoside linking group attached to a nucleoside, a nucleotide, an oligonucleoside, an oligonucleotide, a monomeric subunit or an oligomeric compound; and
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is O, S or NJ1.

In some embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H or C₁-C₆ alkyl, and X is O or NJ1.

In certain such embodiments, at least one Z is C₁-C₆ alkyl or substituted C₁-C₆ alkyl. In certain embodiments, each Z is, independently, C₁-C₆ alkyl or substituted C₁-C₆ alkyl. In certain embodiments, at least one Z is C₁-C₆ alkyl. In certain embodiments, each Z is, independently, C₁-C₆ alkyl. In certain embodiments, at least one Z is methyl. In certain embodiments, each Z is methyl. In certain embodiments, at least one Z is ethyl. In certain embodiments, each Z is ethyl. In certain embodiments, at least one Z is substituted C₁-C₆ alkyl. In certain embodiments, each Z is, independently, substituted C₁-C₆ alkyl. In certain embodiments, at least one Z is substituted methyl. In certain embodiments, each Z is substituted methyl. In certain embodiments, at least one Z is substituted ethyl. In certain embodiments, each Z is substituted ethyl.

In certain embodiments, at least one substituent group is C₁-C₆ alkoxy (e.g., at least one Z is C₁-C₆ alkyl substituted with one or more C₁-C₆ alkoxy). In another embodiment, each substituent group is, independently, C₁-C₆ alkoxy (e.g., each Z is, independently, C₁-C₆ alkyl substituted with one or more C₁-C₆ alkoxy).

In certain embodiments, at least one C₁-C₆ alkoxy substituent group is CH₃O— (e.g., at least one Z is CH₃OCH₂—). In another embodiment, each C₁-C₆ alkoxy substituent group is CH₃O— (e.g., each Z is CH₃OCH₂—).

In certain embodiments, at least one substituent group is halogen (e.g., at least one Z is C₁-C₆ alkyl substituted with one or more halogen). In certain embodiments, each substituent group is, independently, halogen (e.g., each Z is, independently, C₁-C₆ alkyl substituted with one or more halogen). In certain embodiments, at least one halogen substituent group is fluoro (e.g., at least one Z is CH₂FCH₂—, CHF₂CH₂— or CF₃CH₂—). In certain embodiments, each halo substituent group is fluoro (e.g., each Z is, independently, CH₂FCH₂—, CHF₂CH₂— or CF₃CH₂—).

In certain embodiments, at least one substituent group is hydroxyl (e.g., at least one Z is C1-C6 alkyl substituted with one or more hydroxyl). In certain embodiments, each substituent group is, independently, hydroxyl (e.g., each Z is, independently, C₁-C₆ alkyl substituted with one or more hydroxyl). In certain embodiments, at least one Z is HOCH₂—. In another embodiment, each Z is HOCH₂—.

In certain embodiments, at least one Z is CH₃—, CH₃CH₂—, CH₂OCH₃—, CH₂F— or HOCH₂—. In certain embodiments, each Z is, independently, CH₃—, CH₃CH₂—, CH₂OCH₃—, CH₂F— or HOCH₂—.

In certain embodiments, at least one Z group is C₁-C₆ alkyl substituted with one or more Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, at least one Z group is C₁-C₆ alkyl substituted with one or more Xx, wherein each Xx is, independently, halo (e.g., fluoro), hydroxyl, alkoxy (e.g., CH₃O—) or azido.

In certain embodiments, each Z group is, independently, C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, each Z group is, independently, C₁-C₆ alkyl substituted with one or more Xx, wherein each Xx is independently halo (e.g., fluoro), hydroxyl, alkoxy (e.g., CH₃O—) or azido.

In certain embodiments, at least one Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1 In certain embodiments, at least one Z group is —CH₂Xx, wherein Xx is halo (e.g., fluoro), hydroxyl, alkoxy (e.g., CH₃O—) or azido.

In certain embodiments, each Z group is, independently, —CH₂Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, each Z group is, independently, —CH₂Xx, wherein each Xx is, independently, halo (e.g., fluoro), hydroxyl, alkoxy (e.g., CH₃O—) or azido.

In certain embodiments, at least one Z is CH₃—. In another embodiment, each Z is, CH₃—.

In certain embodiments, the Z group of at least one monomer is in the (R)-configuration represented by the formula:

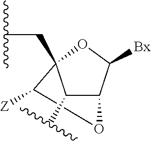

or the formula:

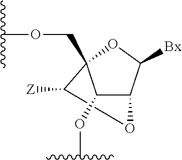

or the formula:

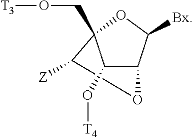

IN certain embodiments, the Z group of each monomer of the formula is in the (R)-configuration.

In certain embodiments, the Z group of at least one monomer is in the (S)-configuration represented by the formula:

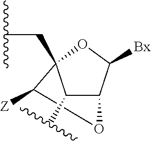

or the formula:

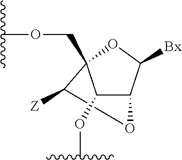

or the formula:

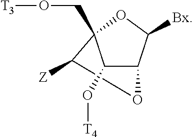

In certain embodiments, the Z group of each monomer of the formula is in the (S)-configuration.

In certain embodiments, T₃ is H or a hydroxyl protecting group. In certain embodiments, T₄ is H or a hydroxyl protecting group. In a further embodiment T₃ is an internucleoside linking group attached to a nucleoside, a nucleotide or a monomeric subunit. In certain embodiments, T₄ is an internucleoside linking group attached to a nucleoside, a nucleotide or a monomeric subunit. In certain embodiments, T₃ is an internucleoside linking group attached to an oligonucleoside or an oligonucleotide. In certain embodiments, T₄ is an internucleoside linking group attached to an oligonucleoside or an oligonucleotide. In certain embodiments, T₃ is an internucleoside linking group attached to an oligomeric compound. In certain embodiments, T₄ is an internucleoside linking group attached to an oligomeric compound. In certain embodiments, at least one of T₃ and T₄ comprises an internucleoside linking group selected from phosphodiester or phosphorothioate.

In certain embodiments, dsRNA agent of the invention comprise at least one region of at least two contiguous monomers of the formula:

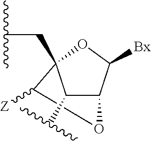

or of the formula:

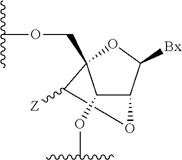

or of the formula:

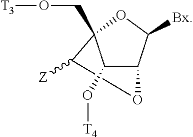

In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)2-O-2′) LNA, (D) Aminooxy (4′-CH₂—O—N(R)-2′) LNA and (E) Oxyamino (4′-CH₂—N(R)—O-2′) LNA, as depicted below:

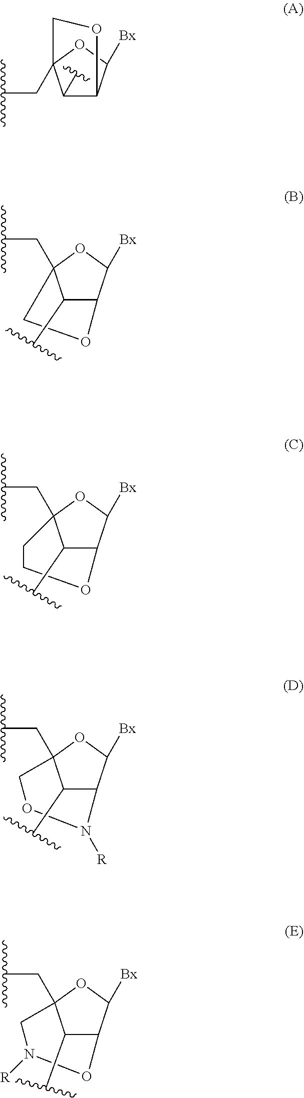

In certain embodiments, the dsRNA agent of the invention comprises at least two regions of at least two contiguous monomers of the above formula. In certain embodiments, the dsRNA agent of the invention comprises a gapped motif. In certain embodiments, the dsRNA agent of the invention comprises at least one region of from about 8 to about 14 contiguous β-D-2′-deoxyribofuranosyl nucleosides. In certain embodiments, the dsRNA agent of the invention comprises at least one region of from about 9 to about 12 contiguous β-D-2′-deoxyribofuranosyl nucleosides.

In certain embodiments, the dsRNA agent of the invention comprises at least one (e.g., 1, 2, 3, 4, 5, 6, 7, 8, 9, 10, 11, 12, 13, 14, 15 or more) comprises at least one (S)-cEt monomer of the formula.

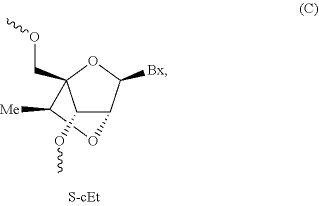

wherein Bₓ is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### C. Intersugar Linkage Modifications

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc.,), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the dsRNA agent of the invention comprises at least one (e.g., 1, 2, 3, 4, 5, 6, 7, 8, 9, 10, 11, 12, 13, 14, 15 or more and upto including all) modified or nonphosphodiester linkages. In some embodiments, the dsRNA agent of the invention comprises at least one (e.g., 1, 2, 3, 4, 5, 6, 7, 8, 9, 10, 11, 12, 13, 14, 15 or more and upto including all) phosphorothioate linkages.

The dsRNA agent of the inventions can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. A preferred surrogate is a PNA surrogate.

The dsRNA agent of the inventions described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the dsRNA agent of the inventions provided herein are all such possible isomers, as well as their racemic and optically pure forms.

### D. Terminal Modifications

In some embodiments, the dsRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the dsRNA agent does not contain a 5′-vinyl phosphonate (VP).

Ends of the iRNA agent of the invention can be modified. Such modifications can be at one end or both ends. For example, the 3′ and/or 5′ ends of an iRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type oligomeric compound.

Terminal modifications useful for modulating activity include modification of the 5′ end of iRNAs with phosphate or phosphate analogs. In certain embodiments, the 5′ end of an iRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

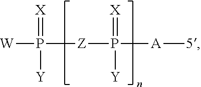

wherein W, X and Y are each independently selected from the group consisting of O, OR (R is hydrogen, alkyl, aryl), S, Se, BR₃ (R is hydrogen, alkyl, aryl), BH3-, C (i.e. an alkyl group, an aryl group, etc.,), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is O, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C₅′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH—5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc.,), 5′-alkyletherphosphonates (R(OH)(O)P—O-5′, R=alkylether, e.g., methoxymethyl (CH₂OMe), ethoxymethyl, etc.,). Other exemplary 5′-modifications include where Z is optionally substituted alkyl at least once, e.g., ((HO)₂(X)P—O[—(CH₂)ₐ-O—P(X)(OH)—O]_{b}—5′, ((HO)₂(X)P—O[—(CH₂)ₐ—P(X)(OH)—O]_{b}—5′, ((HO)₂(X)P—[—(CH₂)ₐ—O—P(X)(OH)—O]_{b}—5′; dialkyl terminal phosphates and phosphate mimics: HO[—(CH₂)ₐ—O—P(X)(OH)—O]_{b}—5′, H₂N[—(CH₂)ₐ—O—P(X)(OH)—O]_{b}—5′, H[—(CH₂)ₐ—O—P(X)(OH)—O]b- 5′, Me₂N[—(CH₂)ₐ—O—P(X)(OH)—O]_{b}—5′, HO[—(CH₂)ₐ—P(X)(OH)—O]_{b}—5′, H₂N[—(CH₂)ₐ—P(X)(OH)—O]b-5′, H[—(CH₂)ₐ—P(X)(OH)—O]_{b}—5′, Me₂N[—(CH₂)ₐ—P(X)(OH)—O]_{b}—5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### E. Thermally Destabilizing Modifications

The compounds of the invention, such as iRNAs or dsRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nucleic acid (GNA).

Exemplified abasic modifications are:

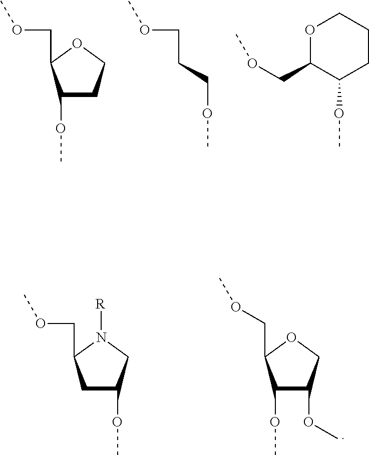

Exemplified sugar modifications are:

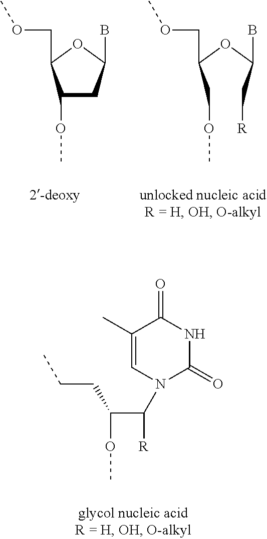

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

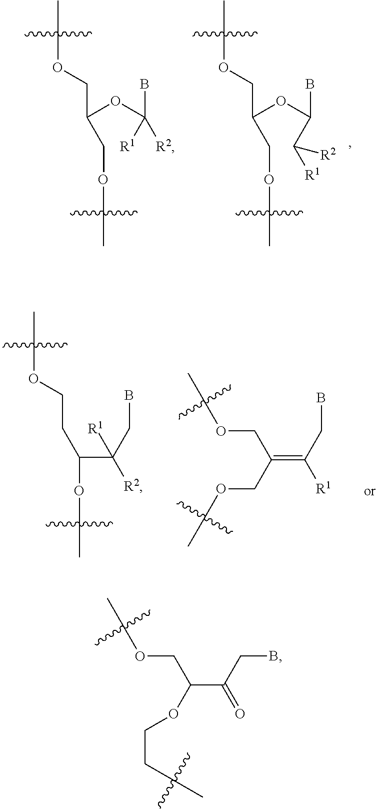

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

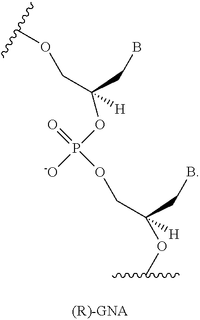

The thermally destabilizing modification can be mismatches (i.e., noncomplementary base pairs) between the rmally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the compounds of the invention, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

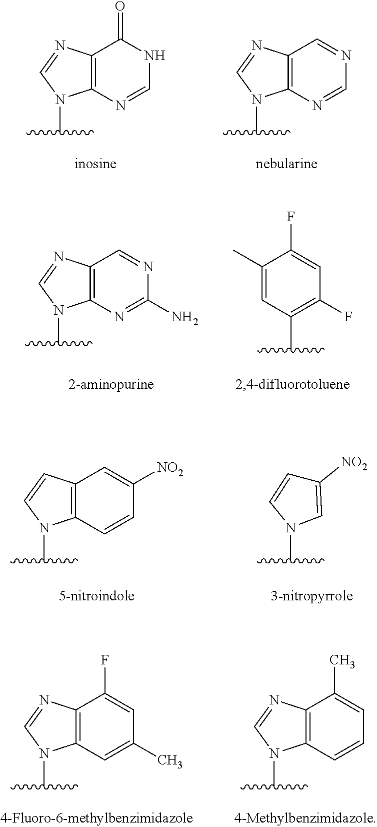

Exemplary phosphate modifications known to decrease the rmal stability of dsRNA duplexes compared to natural phosphodiester linkages are:

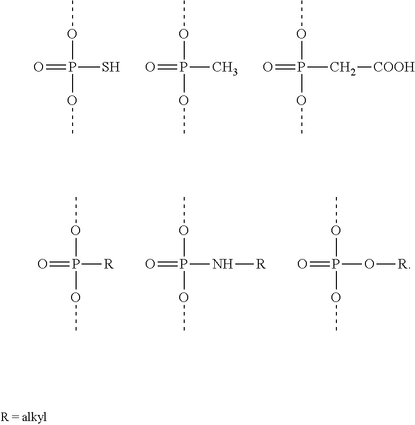

In some embodiments, compounds of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, compounds of the invention can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In one embodiment the iRNA agent of the invention is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

In some embodiments, at least one strand of the iRNA agent of the invention disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH—5′, (HO)(NH2)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH₂—), 5′-alkyletherphosphonates (R=alkylether=methoxymethyl (MeOCH2-), ethoxymethyl, etc., e.g. RP(OH)(O)—O-5′-).

V. Modified RNAi agents of the Invention Comprising Motifs

In certain aspects of the disclosure, the double-stranded RNAi agents of the disclosure include agents with chemical modifications as disclosed, for example, in U.S. Pat. Nos. 9,796,974 and 10,668,170, and U.S. Patent Publication Nos. 2014/288158, 2018/008724, 2019/038768, and 2020/353097, the entire contents of each of which are incorporated herein by reference. As shown therein and in PCT Publication No. WO 2013/074974 (the entire contents of which are incorporated by reference), one or more motifs of three identical modifications on three consecutive nucleotides may be introduced into a sense strand or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense or antisense strand. The RNAi agent may be optionally modified with a (S)-glycol nucleic acid (GNA) modification, for instance on one or more residues of the antisense strand.

In one embodiment, the iRNA agent of the invention is a double ended bluntmer of 19 nt in length, wherein the sense strand contains at least one motif of three 2′-F modifications on three consecutive nucleotides at positions 7, 8, 9 from the 5′ end. The antisense strand contains at least one motif of three 2′-O-methyl modifications on three consecutive nucleotides at positions 11, 12, 13 from the 5′ end.

In one embodiment, the iRNA agent of the invention is a double ended bluntmer of 20 nt in length, wherein the sense strand contains at least one motif of three 2′-F modifications on three consecutive nucleotides at positions 8, 9, 10 from the 5′ end. The antisense strand contains at least one motif of three 2′-O-methyl modifications on three consecutive nucleotides at positions 11, 12, 13 from the 5′ end.

In one embodiment, the iRNA agent of the invention is a double ended bluntmer of 21 nt in length, wherein the sense strand contains at least one motif of three 2′-F modifications on three consecutive nucleotides at positions 9, 10, 11 from the 5′ end. The antisense strand contains at least one motif of three 2′-O-methyl modifications on three consecutive nucleotides at positions 11, 12, 13 from the 5′ end.

In one embodiment, the iRNA agent of the invention comprises a 21 nucleotides (nt) sense strand and a 23 nucleotides (nt) antisense, wherein the sense strand contains at least one motif of three 2′-F modifications on three consecutive nucleotides at positions 9, 10, 11 from the 5′ end; the antisense strand contains at least one motif of three 2′-O-methyl modifications on three consecutive nucleotides at positions 11, 12, 13 from the 5′ end, wherein one end of the iRNA is blunt, while the other end is comprises a 2 nt overhang. Preferably, the 2 nt overhang is at the 3′-end of the antisense.

In one embodiment, the iRNA agent of the invention comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the iRNA agent of the invention comprises a sense and antisense strands, wherein said iRNA agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein said 3′ end of said first strand and said 5′ end of said second strand form a blunt end and said second strand is 1-4 nucleotides longer at its 3′ end than the first strand, wherein the duplex region region which is at least 25 nucleotides in length, and said second strand is sufficiently complemenatary to a target mRNA along at least 19 nt of said second strand length to reduce target gene expression when said iRNA agent is introduced into a mammalian cell, and wherein dicer cleavage of said iRNA preferentially results in an siRNA comprising said 3′ end of said second strand, thereby reducing expression of the target gene in the mammal.

In one embodiment, the sense strand of the iRNA agent contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′ end.

In one embodiment, the antisense strand of the iRNA agent can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′ end.

For iRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the iRNA from the 5′-end.

In some embodiments, the iRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the antisense strand.

In some embodiments, the iRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the iRNA agent comprises a sense strand and antisense strand each having 14 to 30 nucleotides, wherein the sense strand contains at least one motif of three 2′-F modifications on three consecutive nucleotides at positions 9, 10, 11 from the 5′ end, and wherein the antisense strand contains at least one motif of three 2′-O-methyl modifications on three consecutive nucleotides at positions 11, 12, 13 from the 5′ end.

In one embodiment, the iRNA agent of the invention comprises mismatch(es) with the target, within the duplex, or combinations thereof. The mistmatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the iRNA agent of the invention comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxythimidine (dT). In another embodiment, the nucleotide at the 3′-end of the antisense strand is deoxythimidine (dT). In one embodiment, there is a short sequence of deoxythimidine nucleotides, for example, two dT nucleotides on the 3′-end of the sense or antisense strand.

In certain embodiments, the compositions and methods of the disclosure include a vinyl phosphonate (VP) modification of an RNAi agent as described herein. In exemplary embodiments, a 5′-vinyl phosphonate modified nucleotide of the disclosure has the structure:

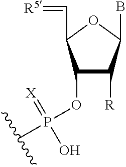

wherein X is O or S;
R is hydrogen, hydroxy, fluoro, or C₁₋₂₀alkoxy (e.g., methoxy or n-hexadecyloxy);
R′ is =C(H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphate modifications are also contemplated for the compositions and methods of the instant disclosure. An exemplary vinyl phosphate structure includes the preceding structure, where R5′ is =C(H)—OP(O)(OH)2 and the double bond between the C5′ carbon and R5′ is in the E or Z orientation (e.g., E orientation).

In one aspect, the invention relates to a double-stranded RNA (dsRNA) agent for inhibiting the expression of a target gene having reduced off-target effects as described in U.S. Pat. Nos. 10,233,448, 10, 612,024, and 10,612,027, and U.S. Patent Publication Nos. 2017/275626, 2019/241891, 2019/241893, and 2021/017519, the entire contents of each of which are incorporated herein by reference. As exemplified therein, a motif comprising, e.g., a thermally destabilizing nucleotide, e.g., i) a nucleotide that forms a mismatch pair with the opposing nucleotide in the antisense strand, ii) a nucleotide having an abasic modification, and/or iii) a nucleotide having a sugar modification, and placed at a site opposite to the seed region (positions 2-8) may be introduced into the sense strand.

In one embodiment, the dsRNA agent of the invention does not contain any 2′-F modification.

In one embodiment, the sense strand and/or antisense strand of the dsRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands of the dsRNA agent has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent of the invention is 100% complementary to a target RNA to hybridize thereto and inhibits its expression through RNA interference. In another embodiment, the antisense strand of the dsRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a dsRNA agent as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand). Each of the embodiments and aspects described in this specification relating to the dsRNA represented by formula (I) can also apply to the dsRNA containing the rmally destabilizing nucleotide.

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′ end of the antisense strand.

In some embodiment, the dsRNA agent as defined herein can comprise i) a phosphorus-containing group at the 5′-end of the sense strand or antisense strand; ii) with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand); and iii) one or more C22 hydrocarbon chains.

In a particular embodiment, the dsRNA agents of the present invention comprising one or more C22 hydrocarbon chains conjugated to one or more internal positions on at least one strand comprise:
(a) a sense strand having:
   (i) a length of 21 nucleotides;
   (ii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
   and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, t the dsRNA agents of the present invention comprising one or more C22 hydrocarbon chains conjugated to one or more internal positions on at least one strand comprise:
(a) a sense strand having:
   (i) a length of 21 nucleotides;
   (ii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (ii) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprising one or more C22 hydrocarbon chains conjugated to one or more internal positions on at least one strand comprise:
(a) a sense strand having:
   (i) a length of 21 nucleotides;
   (ii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
   (ii) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprising one or more C₂₂ hydrocarbon chains conjugated to one or more internal positions on at least one strand comprise:
(a) a sense strand having:
   (i) a length of 21 nucleotides;
   (ii) 2′-OMe modifications at positions 1 to 6, 8, 10, 12, 14, and 16 to 21, and 2′-F modifications at positions 7, 9, 11, 13, and 15; and
   (ii) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, t the dsRNA agents of the present invention comprising one or more C₂₂ hydrocarbon chains conjugated to one or more internal positions on at least one strand comprise:
(a) a sense strand having:
   (i) a length of 21 nucleotides;
   (ii) 2′-OMe modifications at positions 1 to 9, and 12 to 21, and 2′-F modifications at positions 10, and 11; and
   (ii) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprising one or more C22 hydrocarbon chains conjugated to one or more internal positions on at least one strand comprise:
(a) a sense strand having:
   (i) a length of 21 nucleotides;
   (ii) 2′-F modifications at positions 1, 3, 5, 7, 9 to 11, and 13, and 2′-OMe modifications at positions 2, 4, 6, 8, 12, and 14 to 21; and
   (ii) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprising one or more C22 hydrocarbon chains conjugated to one or more internal positions on at least one strand comprise:
(a) a sense strand having:
   (i) a length of 21 nucleotides;
   (ii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (ii) phosphorothioate internucleotide linkages between nucleotide positions 1 and 2, and between nucleotide positions 2 and 3 (counting from the 5′ end);
   and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a four nucleotide overhang at the 3′-end of the antisense strand, and a blunt end at the 5′-end of the antisense strand.

In another particular embodiment, the dsRNA agents of the present invention comprising one or more C22 hydrocarbon chains conjugated to one or more internal positions on at least one strand comprise:
(a) a sense strand having:
   (i) a length of 21 nucleotides;
   (ii) 2′-OMe modifications at positions 1 to 6, 8, and 12 to 21, and 2′-F modifications at positions 7, and 9 to 11; and
   (ii) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprising one or more C22 hydrocarbon chains conjugated to one or more internal positions on at least one strand comprise:
(a) a sense strand having:
   (i) a length of 21 nucleotides;
   (ii) 2′-OMe modifications at positions 1 to 6, 8, and 12 to 21, and 2′-F modifications at positions 7, and 9 to 11; and
   (ii) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprising one or more C22 hydrocarbon chains conjugated to one or more internal positions on at least one strand comprise:
(a) a sense strand having:
   (i) a length of 19 nucleotides;
   (ii) 2′-OMe modifications at positions 1 to 4, 6, and 10 to 19, and 2′-F modifications at positions 5, and 7 to 9; and
   (ii) phosphorothioate internucleotide linkages between nucleotide positions 1 and 2, and between nucleotide positions 2 and 3 (counting from the 5′ end);
   and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
wherein the dsRNA agents have a two nucleotide overhang at the 3′-end of the antisense strand, and a blunt end at the 5′-end of the antisense strand.

In one embodiment, the dsRNA agents of the present invention comprising one or more C22 hydrocarbon chains conjugated to one or more internal positions on at least one strand comprise:
(a) a sense strand having:
   (i) a length of 18-23 nucleotides;
   (ii) three consecutive 2′-F modifications at positions 7-15; and
(b) an antisense strand having:
   (i) a length of 18-23 nucleotides;
   (ii) at least 2′-F modifications anywhere on the strand; and
   (iii) at least two phosphorothioate internucleotide linkages at the first five nucleotides (counting from the 5′ end);
   and either have two nucleotides overhang at the 3′-end of the antisense strand, and a blunt end at the 5′-end of the antisense strand; or blunt end both ends of the duplex.

In one embodiment, the dsRNA agents of the present invention comprise:
(a) a sense strand having:
   (i) a length of 18-23 nucleotides;
   (ii) less than four 2′-F modifications;
(b) an antisense strand having:
   (i) a length of 18-23 nucleotides;
   (ii) at less than twelve 2′-F modification; and
   (iii) at least two phosphorothioate internucleotide linkages at the first five nucleotides (counting from the 5′ end);
wherein the dsRNA agents have either have two nucleotides overhang at the 3′-end of the antisense strand, and a blunt end at the 5′-end of the antisense strand; or blunt end both ends of the duplex.

In one embodiment, the dsRNA agents of the present invention comprise:
(a) a sense strand having:
   (i) a length of 19-35 nucleotides;
   (ii) less than four 2′-F modifications;
(b) an antisense strand having:
   (i) a length of 19-35 nucleotides;
   (ii) at less than twelve 2′-F modification; and
   (iii) at least two phosphorothioate internucleotide linkages at the first five nucleotides (counting from the 5′ end);
wherein the duplex region is between 19 to 25 base pairs (preferably 19, 20, 21 or 22); and wherein the dsRNA agents have either have two nucleotides overhang at the 3′-end of the antisense strand, and a blunt end at the 5′-end of the antisense strand; or blunt end both ends of the duplex.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); and wherein the dsRNA agents have less than 20%, less than 15% and less than 10% non-natural nucleotide.

Examples of non-natural nucleotide includes acyclic nucleotides, LNA, HNA, CeNA, 2′-methoxyethyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F, and others.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have greater than 80%, greater than 85% and greater than 90% natural nucleotide, such as 2′-OH, 2′-deoxy and 2′-OMe are natural nucleotides.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); and wherein the dsRNA agents have 100% natural nucleotide, such as 2′-OH, 2′-deoxy and 2′-OMe are natural nucleotides.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, 100%, 95%, 90%, 85%, 80%, 75%, 70%, 65%, 60%, 55%, 50%, 45%, 40%, 35% or 30% of the iRNA agent of the invention is modified.

In some embodiments, each of the sense and antisense strands of the iRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In some embodiments, each of the sense and antisense strands of the iRNA agent contains at least two different modifications.

In some embodiments, the dsRNA agent of the invention of the invention does not contain any 2′-F modification.

In some embodiments, the dsRNA agent of the invention contains one, two, three, four, five, six, seven, eight, nine, ten, eleven or twelve 2′-F modification(s). In one example, dsRNA agent of the invention contains nine or ten 2′-F modifications.

The iRNA agent of the invention may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the iRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paried nucleotide next to the overhang nucleotide. Preferably, these terminal three nucleotides may be at the 3′-end of the antisense strand.

In some embodiments, the sense strand and/or antisense strand of the iRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In some embodiments, the antisense strand of the iRNA agent of the invention is 100% complementary to a target RNA to hybridize thereto and inhibits its expression through RNA interference. In another embodiment, the antisense strand of the iRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a iRNA agent capable of inhibiting the expression of a target gene. The iRNA agent comprises a sense strand and an antisense strand, each strand having 14 to 40 nucleotides. The sense strand contains at least one thermally destabilizing nucleotide, wherein at at least one said thermally destabilizing nucleotide occurs at or near the site that is opposite to the seed region of the antisense strand (i.e. at position 2-8 of the 5′-end of the antisense strand), For example, the rmally destabilizing nucleotide occurs between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that is smaller than a sterically demanding 2′-OMe are separated by 11 nucleotides in length. For example, the two modified nucleic acids are at positions 2 and 14 of the 5′ end of the antisense strand.

In some embodiments, the compound of the invention disclosed herein is a miRNA mimic. In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded iRNAs. In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

### VI. Synthesis of RNAi Agents of the Invention

The nucleic acids featured in the disclosure can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

An siRNA can be produced, e.g., in bulk, by a variety of methods. Exemplary methods include: organic synthesis and RNA cleavage, e.g., in vitro cleavage.

### A. Organic Synthesis

An siRNA can be made by separately synthesizing a single stranded RNA molecule, or each respective strand of a double-stranded RNA molecule, after which the component strands can then be annealed.

A large machine, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

B. dsiRNA Cleavage

siRNAs can also be made by cleaving a larger siRNA. The cleavage can be mediated in vitro or in vivo. For example, to produce iRNAs by cleavage in vitro, the following method can be used:

1. In vitro transcription.

dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes. In one embodiment, RNA generated by this method is carefully purified to remove endotoxins that may contaminate preparations of the recombinant enzymes.

### 2. In Vitro Cleavage.

dsRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9 and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

C. Making dsRNA Agents Conjugated to One or More C₂₂ Hydrocarbon Chains

In some embodiments, the one or more C₂₂ hydrocarbon chains is conjugated to the dsRNA agent via a nucleobase, sugar moiety, or internucleosidic linkage.

Conjugation to purine nucleobases or derivatives thereof can occur at any position including, endocyclic and exocyclic atoms. In some embodiments, the 2-, 6-, 7-, or 8-positions of a purine nucleobase are attached to a C₂₂ hydrocarbon chain. Conjugation to pyrimidine nucleobases or derivatives thereof can also occur at any position. In some embodiments, the 2-, 5-, and 6-positions of a pyrimidine nucleobase can be substituted with a C₂₂ hydrocarbon chain. When one or more C22 hydrocarbon chains is conjugated to a nucleobase, the preferred position is one that does not interfere with hybridization, i.e., does not interfere with the hydrogen bonding interactions needed for base pairing. In one embodiment, the one or more C₂₂ hydrocarbon chains may be conjugated to a nucleobase via a linker containing an alkyl, alkenyl or amide linkage.

Conjugation to sugar moieties of nucleosides can occur at any carbon atom. Exemplary carbon atoms of a sugar moiety that the one or more C₂₂ hydrocarbon chains can be attached to include the 2′, 3′, and 5′ carbon atoms. The one or more C₂₂ hydrocarbon chains can also be attached to the 1′ position, such as in an abasic residue. In one embodiment, the one or more C22 hydrocarbon chains may be conjugated to a sugar moiety, via a 2′-Omodification, with or without a linker.

Internucleosidic linkages can also bear the one or more C22 hydrocarbon chains. For phosphorus-containing linkages (e.g., phosphodiester, phosphorothioate, phosphorodithiotate, phosphoroamidate, and the like), the one or more C22 hydrocarbon chains can be attached directly to the phosphorus atom or to an O, N, or S atom bound to the phosphorus atom. For amine- or amide-containing internucleosidic linkages (e.g., PNA), the one or more C22 hydrocarbon chains can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In one embodiment, a first (complementary) RNA strand and a second (sense) RNA strand can be synthesized separately, wherein one of the RNA strands comprises a pendant C₂₂ hydrocarbon chain, and the first and second RNA strands can be mixed to form a dsRNA. The step of synthesizing the RNA strand preferably involves solid-phase synthesis, wherein individual nucleotides are joined end to end through the formation of internucleotide 3′-5′ phosphodiester bonds in consecutive synthesis cycles.

In one embodiment, the C₂₂ hydrocarbon chain having a phosphoramidite group is coupled to the 3′-end or 5′-end of either the first (complementary) or second (sense) RNA strand in the last synthesis cycle. In the solid-phase synthesis of an RNA, the nucleotides are initially in the form of nucleoside phosphoramidites. In each synthesis cycle, a further nucleoside phosphoramidite is linked to the —OH group of the previously incorporated nucleotide. If the one or more C22 hydrocarbon chains has a phosphoramidite group, it can be coupled in a manner similar to a nucleoside phosphoramidite to the free OH end of the RNA synthesized previously in the solid-phase synthesis. The synthesis can take place in an automated and standardized manner using a conventional RNA synthesizer. Synthesis of the molecule having the phosphoramidite group may include phosphitylation of a free hydroxyl to generate the phosphoramidite group.

Synthesis procedures of the one or more C22 hydrocarbon chain-conjugated phosphoramidites are exemplified in Example 1.

In general, the oligonucleotides can be synthesized using protocols known in the art, for example, as described in Caruthers et al., Methods in Enzymology (1992) 211:3-19; WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and U.S. Pat. No. 6,001,311; each of which is hereby incorporated by reference in its entirety. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland, Mass.). Alternatively, syntheses can be performed on a 96-well plate synthesizer, such as the instrument produced by Protogene (Palo Alto, Calif), or by methods such as those described in Usman et al., J. Am. Chem. Soc. (1987) 109:7845; Scaringe, et al., Nucl. Acids Res. (1990) 18:5433; Wincott, et al., Nucl. Acids Res. (1990) 23:2677-2684; and Wincott, et al., Methods Mol. Bio. (1997) 74:59, each of which is hereby incorporated by reference in its entirety.

The nucleic acid molecules of the present invention may be synthesized separately and joined together post-synthetically, for example, by ligation (Moore et al., Science (1992) 256:9923; WO 93/23569; Shabarova et al., Nucl. Acids Res. (1991) 19:4247; Bellon et al., Nucleosides & Nucleotides (1997) 16:951; Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

### VII. Ligands

In certain embodiments, the dsRNA agent of the invention is further modified by covalent attachment of one or more conjugate groups. In general, conjugate groups modify one or more properties of the attached dsRNA agent of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the dsRNA agent further comprises a targeting ligand that targets a receptor which mediates delivery to a specific muscle or adipose tissue. These targeting ligands can be conjugated by otself or in combination with the one or more C₂₂ hydrocarbon chains to enable specific systemic delivery.

Exemplary targeting ligands that targets the receptor mediated delivery to a muscle or adipose tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); athioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol ortriethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)qlycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes oftetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

AALEALAEALEALAEALEALAEAAAAGGC (GALA);
AALAEALAEALAEALAEALAEALAAAAGGC (EALA);
ALEALAEALEALAEA;
GLFEAIEGFIENGWEGMIWDYG (INF-7);
GLFGAIAGFIENGWEGMIDGWYG (Inf HA-2);
GLFEAIEGFIENGWEGMIDGWYGCGLFEAIEGFIENGWEGMID GWYGC
(diINF-7);
GLFEAIEGFIENGWEGMIDGGCGLFEAIEGFIENGWEGMIDGGC
(diINF-3);
GLFGALAEALAEALAEHLAEALAEALEALAAGGSC (GLF);
GLFEAIEGFIENGWEGLAEALAEALEALAAGGSC (GALA-INF3);
GLF EAI EGFI ENGW EGnI DG K GLF EAI EGFI ENGW
EGnI DG (INF-5, n is norleucine);
LFEALLELLESLWELLLEA (JTS-1);
GLFKALLKLLKSLWKLLLKA (ppTG1);
GLFRALLRLLRSLWRLLLRA (ppTG20);
WEAKLAKALAKALAKHLAKALAKALKACEA (KALA);
GLFFEAIAEFIEGGWEGLIEGC (HA);
GIGAVLKVLTTGLPALISWIKRKRQQ (Melittin);
H₅WYG;
and
CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also refered to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to,

RQIKIWFQNRRMKWKK (penetratin);
GRKKRRQRRRPPQC (Tat fragment 48-60);
GALFLGWLGAAGSTMGAWSQPKKKRKV (signal sequence based
peptide);
LLIILRRRIRKQAHAHSK (PVEC);
GWTLNSAGYLLKINLKALAALAKKIL (transportan);
KLALKLALKALKAALKLA (amphiphilic model peptide);
RRRRRRRRR (Arg9);
KFFKFFKFFK (Bacterial cell wall permeating
peptide);
LLGDFFRKSKEKIGKEFKRIVQRIKDFLRNLVPRTES (LL-37);
SWLSKTAKKLENSAKKRISEGIAIAIQGGPR (cecropin P1);
ACYCRIPACIAGERRYGTCIYQGRLWAFCC (α-defensin);
DHYNCVSSGGQCLYSACPIFTKIQGTCYRGKAKCCK (β-defensin);
RRRPRPPYLPRPRPPPFFPPRLPPRIPPGFPPRFPPRFPGKR-NH2
(PR-39);
ILPWKWPWWPWRR-NH2 (indolicidin);
AAVALLPAVLLALLAP (RFGF);
AALLPVLLAAP (RFGF analogue);
and
RKCRIVVIRVCR (bactenecin).

Exemplary cationic groups include, but are not limited to, protonated amino groups, derived from e.g., β-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)˜CH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-glucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, scuh as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the dsRNA agent of the invention (e.g., a dsRNA agent of the invention or linker). In some embodiments, the ligand can be incorporated via coupling to a “precursor” monomer after said “precursor” monomer has been incorporated into a component of the dsRNA agent of the invention (e.g., a dsRNA agent of the invention or linker). For example, a monomer having, e.g., an amino-terminated tether (i.e., having no associated ligand), e.g., monomer-linker-NH₂ can be incorporated into a component of the compounds of the invention (e.g., a dsRNA agent of the invention or linker). In a subsequent operation, i.e., after incorporation of the precursor monomer into a component of the compounds of the invention (e.g., a dsRNA agent of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the dsRNA agent of the invention. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the dsRNA agent of the inventions via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218, 105; 5,525,465; 5,541,313; 5,545,730; 5,552,538; 5,578, 717, 5,580,731; 5,580,731; 5,591,584; 5,109,124; 5,118, 802; 5,138,045; 5,414,077; 5,486,603; 5,512,439; 5,578, 718; 5,608,046; 4,587,044; 4,605,735; 4,667,025; 4,762, 779; 4,789,737; 4,824,941; 4,835,263; 4,876,335; 4,904, 582; 4,958,013; 5,082,830; 5,112,963; 5,214,136; 5,082, 830; 5,112,963; 5,149,782; 5,214,136; 5,245,022; 5,254, 469; 5,258,506; 5,262,536; 5,272,250; 5,292,873; 5,317,098; 5,371,241, 5,391,723; 5,416,203, 5,451,463; 5,510, 475; 5,512,667; 5,514,785; 5,565,552; 5,567,810; 5,574, 142; 5,585,481; 5,587,371; 5,595,726; 5,597,696; 5,599, 923; 5,599,928; 5,672,662; 5,688,941; 5,714,166; 6,153, 737; 6,172,208; 6,300,319; 6,335,434; 6,335,437; 6,395, 437; 6,444,806; 6,486,308; 6,525,031; 6,528,631; 6,559, 279; contents of which are herein incorporated in their entireties by reference.

In some embodiments, the dsRNA agent further comprises a targeting ligand that targets a liver tissue. In some embodiments, the targeting ligand is a carbohydrate-based ligand. In one embodiment, the targeting ligand is a GalNAc conjugate.

In certain embodiments, the dsRNA agent of the invention further comprises a ligand having a structure shown below:

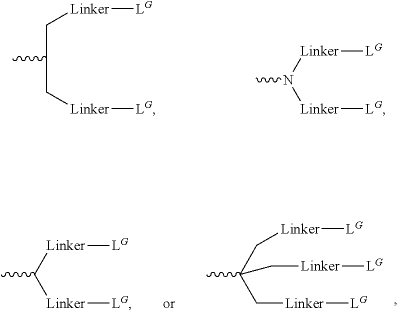

wherein:
   L^{G} is independently for each occurrence a ligand, e.g., carbohydrate, e.g. monosaccharide, disaccharide, trisaccharide, tetrasaccharide, polysaccharide; and
   Z′, Z″, Z″′ and Z″″ are each independently for each occurrence O or S.

In certain embodiments, the dsRNA agent of the invention comprises a ligand of Formula (II), (III), (IV) or (V):

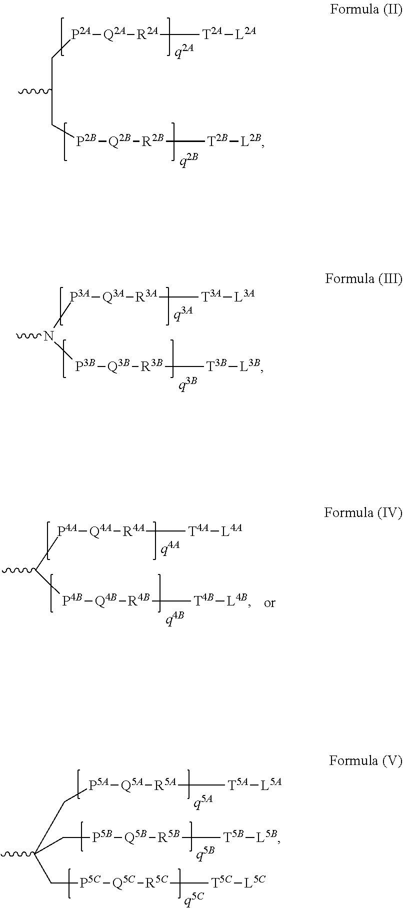

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
Q and Q′ are independently for each occurrence is absent, —(P⁷-Q⁷-R⁷)ₚ-T⁷- or -T⁷-Q⁷-T^{7′}-B-T⁸′-Q⁸-T⁸;
P^{2A}, P^{2B}, P^{3A}, P^{3B}, P^{4A}, P^{4B}, P^{5A}, P^{5B}, P^{5C} P⁷, T^{2A}, T^{2B}, T^{3A}, T^{3B}, T^{4A}, T^{4B} T^{4A} T^{5B} T^{5C} T⁷, T⁷, T⁸ and T^{8′} are each independently for each occurrence absent, CO, NH, O, S, OC(O), NHC(O), CH₂, CH₂NH or CH₂O;
B is —CH₂—N(B^{L})—CH₂—;
B^{L} is -T^{B}-Q^{B}-T^{B′}-R
   Q^{2A}, Q^{2B}, Q^{3A}, Q^{3B}, Q^{4A}, Q^{4B}, Q^{5A}, Q^{5B}, Q⁵c Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C—C or C(O);
   T^{B} and T^{B′} are each independently for each occurrence absent, CO, NH, O, S, OC(O), OC(O)O, NHC(O), NHC(O)NH, NHC(O)O, CH₂, CH₂NH or CH₂O;
   R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), aterpene (e.g., triterpene, e.g., sarsasapogenin, Friedelin, epifriedelanol derivatized lithocholic acid), or a cationic lipid;
   R¹, R², R^{2A}, R^{2B}, R³R^{3B}, R⁴R^{4B}, R^{5A}, R^{5B}, R^{5c}, R′ are each independently for each occurrence absent, NH, O, S, CH₂, C(O)O, C(O)NH, NHCH(R^{a})C(O), —C(O)—CH(R^{a})—NH—, CO, CH═N—O,

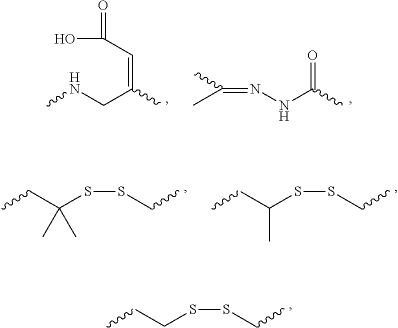

or heterocyclyl;
L, L^{2A}, L^{2B}, L^{3A}, L^{3B}, L^{4A}, L^{4B}, L⁵A, L^{5B} and L^{5C} are each independently for each occurrence a carbohydrate, e.g., monosaccharide, disaccharide, trisaccharide, tetrasaccharide, oligosaccharide and polysaccharide;
R′ and R″ are each independently H, C₁-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z″′ and Z″″ are each independently for each occurrence O or S;
p represent independently for each occurrence 0-20.

As discussed above, because the ligand can be conjugated to the iRNA agent via a linker or carrier, and because the linker or carrier can contain a branched linker, the iRNA agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valencies. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

Suitable ligands for use in the compositions of the invention are described in U.S. Pat. Nos. 8,106,022, 8,450,467, 8,882,895, 9,352,048, 9,370,581, 9,370,582, 9,867,882, 10,806,791, and 11,110,174, and U.S. Patent Publication Nos. 2009/239814, 200/9247608, 2012/136042, 2013/178512, 2014/179761, 2015/011615, 2015/119444, 2015/119445, 2016/051691, 2016/375137, 2018/326070, 2019/099493, 2019/184018, and 2020/297853, the entire contents of each of which are incorporated herein by reference.

In some embodiments, a suitable ligand is a ligand disclosed in WO 2019/055633, the entire contents of which are incorporated herein by reference. In one embodiment the ligand comprises the structure below:

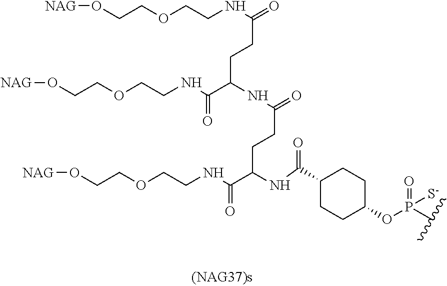

In certain embodiments, the dsRNA agent of the invention comprises a ligand of structure:

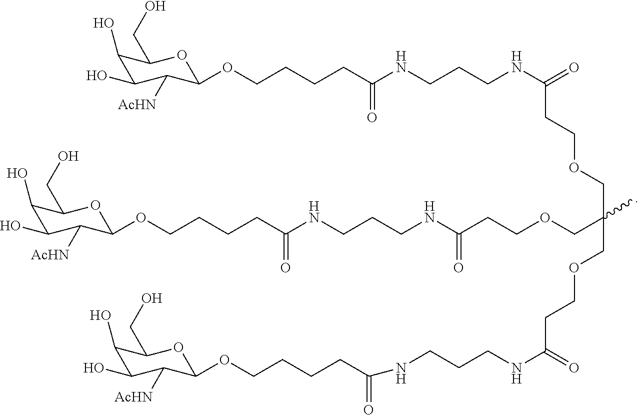 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the dsRNA agent of the invention is conjugated with a ligand of structure:

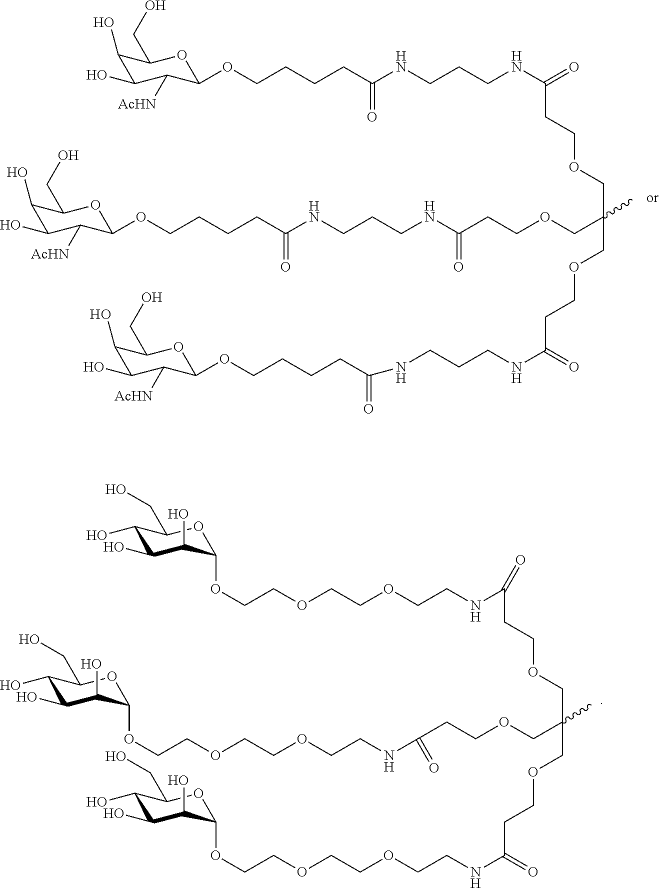

In certain embodiments, the dsRNA agent of the invention comprises a ligand of structure:

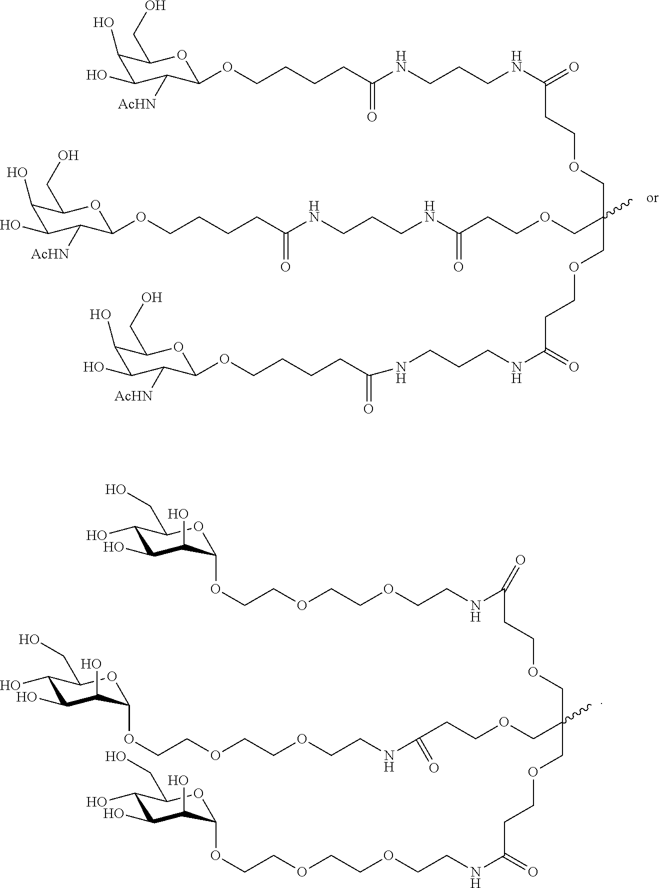

In certain embodiments, the dsRNA agent of the invention comprises a monomer of structure:

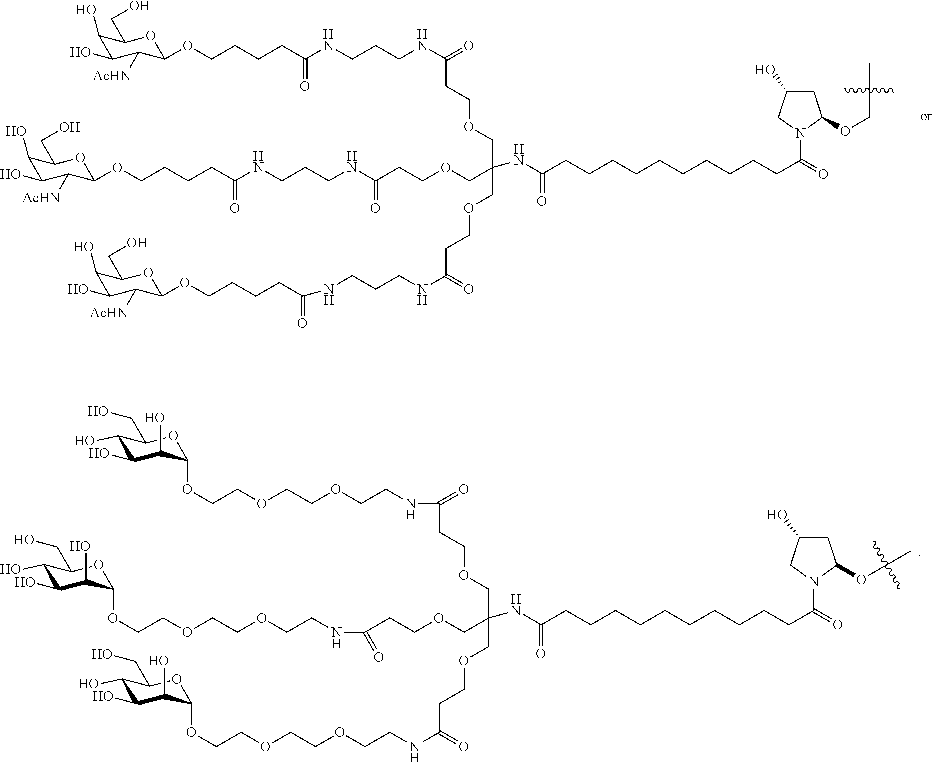

In some embodiments, the RNAi agent is attached to the carbohydrate conjugate via a linker as shown in the following schematic, wherein X is O or S

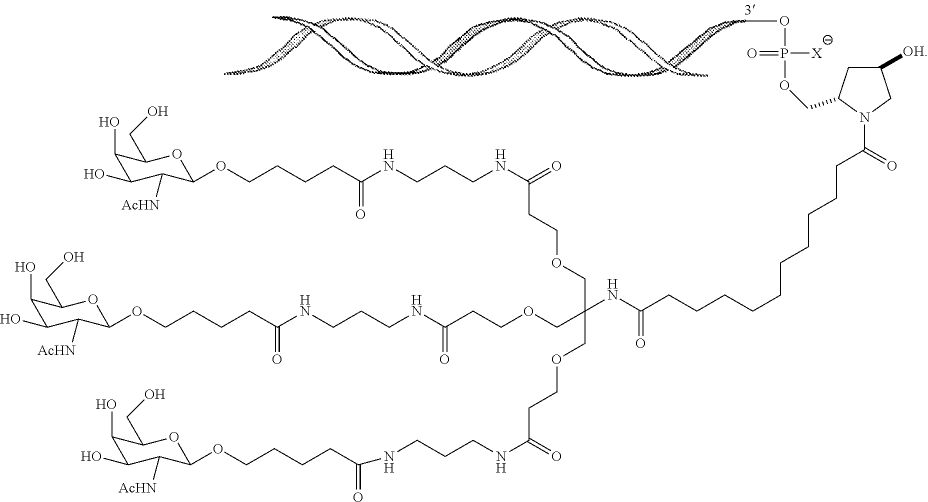

In some embodiments, the RNAi agent is conjugated to L96 as defined in Table 1 and shown below:

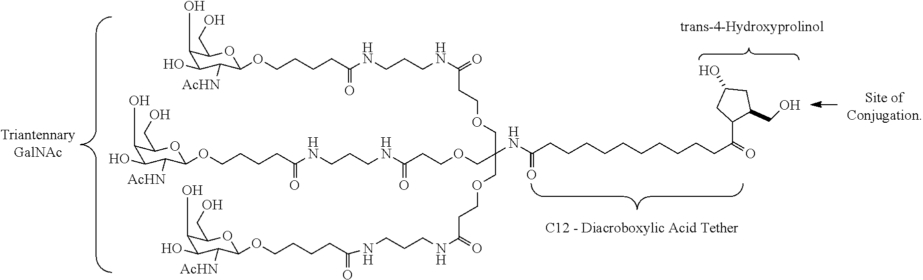

Synthesis of above described ligands and monomers is described, for example, in U.S. Pat. No. 8,106,022, content of which is incorporated herein by reference in its entirety.

### VIII. Delivery of an RNAi Agent of the Disclosure

The delivery of a RNAi agent of the disclosure to a cell e.g., a cell within a subject, such as a human subject (e.g., a subject in need thereof, such as a subject having a target gene-associated disorder, can be achieved in a number of different ways. For example, delivery may be performed by contacting a cell with an RNAi agent of the disclosure either in vitro or in vivo. In vivo delivery may also be performed directly by administering a composition comprising an RNAi agent, e.g., a dsRNA, to a subject. Alternatively, in vivo delivery may be performed indirectly by administering one or more vectors that encode and direct the expression of the RNAi agent. These alternatives are discussed further below.

In general, any method of delivering a nucleic acid molecule (in vitro or in vivo) can be adapted for use with a RNAi agent of the disclosure (see e.g., Akhtar S. and Julian R L, (1992) Trends Cell. Biol. 2(5):139-144 and WO94/02595, which are incorporated herein by reference in their entireties). For in vivo delivery, factors to consider in order to deliver an RNAi agent include, for example, biological stability of the delivered agent, prevention of non-specific effects, and accumulation of the delivered agent in the target tissue. The non-specific effects of an RNAi agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. Several studies have shown successful knockdown of gene products when an RNAi agent is administered locally. For example, pulmonary delivery, e.g., inhalation, of a dsRNA, e.g., SOD1, has been shown to effectively knockdown gene and protein expression in lung tissue and that there is excellent uptake of the dsRNA by the bronchioles and alveoli of the lung. Intraocular delivery of a VEGF dsRNA by intravitreal injection in cynomolgus monkeys (Tolentino, M J. et al., (2004) Retina 24:132-138) and subretinal injections in mice (Reich, S J. et al. (2003) Mol. Vis. 9:210-216) were also both shown to prevent neovascularization in an experimental model of age-related macular degeneration. In addition, direct intratumoral injection of a dsRNA in mice reduces tumor volume (Pille, J. et al. (2005) Mol. Ther. 11:267-274) and can prolong survival of tumor-bearing mice (Kim, W J. et al., (2006) Mol. Ther. 14:343-350; Li, S. et al., (2007) Mol. Ther. 15:515-523). RNA interference has also shown success with local delivery to the lungs by intranasal administration (Howard, K A. et al., (2006) Mol. Ther. 14:476-484; Zhang, X. et al., (2004) J. Biol. Chem. 279:10677-10684; Bitko, V. et al., (2005) Nat. Med. 11:50-55). For administering a RNAi agent systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the RNAi agent to the target tissue and avoid undesirable off-target effects (e.g., without wishing to be bound by theory, use of GNAs as described herein has been identified to destabilize the seed region of a dsRNA, resulting in enhanced preference of such dsRNAs for on-target effectiveness, relative to off-target effects, as such off-target effects are significantly weakened by such seed region destabilization). RNAi agents can be modified by chemical conjugation to lipophilic groups such as cholesterol to enhance cellular uptake and prevent degradation. For example, a RNAi agent directed against ApoB conjugated to a lipophilic cholesterol moiety was injected systemically into mice and resulted in knockdown of apoB mRNA in both the liver and jejunum (Soutschek, J. et al., (2004) Nature 432:173-178). Conjugation of an RNAi agent to an aptamer has been shown to inhibit tumor growth and mediate tumor regression in a mouse model of prostate cancer (McNamara, J O. et al., (2006) Nat. Biotechnol. 24:1005-1015). In an alternative embodiment, the RNAi agent can be delivered using drug delivery systems such as a nanoparticle, a dendrimer, a polymer, liposomes, or a cationic delivery system. Positively charged cationic delivery systems facilitate binding of molecule RNAi agent (negatively charged) and also enhance interactions at the negatively charged cell membrane to permit efficient uptake of an RNAi agent by the cell. Cationic lipids, dendrimers, or polymers can either be bound to an RNAi agent, or induced to form a vesicle or micelle (see e.g., Kim S H. et al., (2008) Journal of Controlled Release 129(2):107-116) that encases an RNAi agent. The formation of vesicles or micelles further prevents degradation of the RNAi agent when administered systemically. Methods for making and administering cationic-RNAi agent complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of RNAi agents include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005)IntJ. Oncol. 26:1087-1091), polyethyleneimine (Bonnet M E. et al., (2008) Pharm. Res. August 16 Epub ahead of print; Aigner, A. (2006) J Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a RNAi agent forms a complex with cyclodextrin for systemic administration. Methods for administration and pharmaceutical compositions of RNAi agents and cyclodextrins can be found in U.S. Pat. No. 7,427,605, which is herein incorporated by reference in its entirety.

Certain aspects of the instant disclosure relate to a method of reducing the expression of a target gene in a cell, comprising contacting said cell with the double-stranded RNAi agent of the disclosure. In one embodiment, the cell is a skeletal muscle. In one embodiment, the cell is a cardiac muscle cell. In one embodiment, the cell is an adipocyte.

In certain embodiments, the RNAi agent is taken up on one or more tissue or cell types present in organs, e.g., liver, skeletal muscle, cardiac muscle, adipose tissue.

Another aspect of the disclosure relates to a method of reducing the expression and/or activity of a target gene in a subject, comprising administering to the subject the double-stranded RNAi agent of the disclosure.

Another aspect of the disclosure relates to a method of treating a subject having a target gene-associated disorder or at risk of having or at risk of developing a target gene-associated disorder, comprising administering to the subject a therapeutically effective amount of the double-stranded RNAi agent of the disclosure, thereby treating the subject.

In one embodiment, the double-stranded RNAi agent is administered subcutaneously.

In one embodiment, the double-stranded RNAi agent is administered intramuscularly.

In one embodiment, the double-stranded RNAi agent is administered by intravenously.

In one embodiment, the double-stranded RNAi agent is administered by pulmonary system administration, e.g., intranasal administration, or oral inhalative administration.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the disclosure. A composition that includes a RNAi agent can be delivered to a subject by a variety of routes. Exemplary routes include pulmonary system, intravenous, subcutaneous, oral, topical, rectal, anal, vaginal, nasal, and ocular.

The RNAi agents of the disclosure can be incorporated into pharmaceutical compositions suitable for administration. Such compositions typically include one or more species of RNAi agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present disclosure may be administered in a number of ways depending upon whether local or systemic treatment is desired and upon the area to be treated. Administration may be intratracheal, intranasal, topical (including ophthalmic, vaginal, rectal, intranasal, transdermal), oral, parenteral, or pulmonary, e.g., by inhalation or insufflation of powders or aerosols, including by nebulizer. Parenteral administration includes intravenous drip, subcutaneous, intraperitoneal, or intramuscular injection administration.

The route and site of administration may be chosen to enhance targeting. For example, to target muscle cells, intramuscular injection into the muscles of interest would be a logical choice. Lung cells might be targeted by administering the RNAi agent in powder or aerosol form. The vascular endothelial cells could be targeted by coating a balloon catheter with the RNAi agent and mechanically introducing the RNA.

Compositions for pulmonary system delivery may include aqueous solutions, e.g., for intranasal or oral inhalative administration, suitable carriers composed of, e.g., lipids (liposomes, niosomes, microemulsions, lipidic micelles, solid lipid nanoparticles) or polymers (polymer micelles, dendrimers, polymeric nanoparticles, nonogels, nanocapsules), adjuvant, e.g., for oral inhalative administration. Aqueous compositions may be sterile and may optionally contain buffers, diluents, absorbtion enhancers and other suitable additives. Such administration permits both systemic and local delivery of the double stranded RNAi agents of the invention.

Intranasal administration may include instilling or insufflating a double stranded RNAi agent into the nasal cavity with syringes or droppers by applying a few drops at a time or via atomization. Suitable dosage forms for intranasal administration include drops, powders, nebulized mists, and sprays. Nasal delivery devices include, but not limited to, vapor inhaler, nasal dropper, spray bottle, metered dose spray pump, gas driven spray atomizer, nebulizer, mechanical powder sprayer, breath actuated inhaler, and insufflator. Devices for delivery deeper into the respiratory system, e.g., into the lung, include nebulizer, pressured metered-dose inhaler, dry powder inhaler, and thermal vaporization aerosol device. Devices for delivery by inhalation are available from commercial suppliers. Devices can be fixed or variable dose, single or multidose, disposable or reusable depending on, for example, the disease or disorder to be prevented or treated, the volume of the agent to be delivered, the frequency of delivery of the agent, and other considerations in the art.

Oral inhalative administration may include use of device, e.g., a passive breath driven or active power driven single/-multiple dose dry powder inhaler (DPI), to deliver a double stranded RNAi agent to the pulmonary system. Suitable dosage forms for oral inhalative administration include powders and solutions. Suitable devices for oral inhalative administration include nebulizers, metered-dose inhalers, and dry powder inhalers. Dry powder inhalers are of the most popular devices used to deliver drugs, especially proteins to the lungs. Exemplary commercially available dry powder inhalers include Spinhaler (Fisons Pharmaceuticals, Rochester, NY) and Rotahaler (GSK, RTP, NC). Several types of nebulizers are available, namely jet nebulizers, ultrasonic nebulizers, vibrating mesh nebulizers. Jet nebulizers are driven by compressed air. Ultrasonic nebulizers use a piezoelectric transducer in order to create droplets from an open liquid reservoir. Vibrating mesh nebulizers use perforated membranes actuated by an annular piezoelement to vibrate in resonant bending mode. The holes in the membrane have a large cross-section size on the liquid supply side and a narrow cross-section size on the side from where the droplets emerge. Depending on the rapeutic application, the hole sizes and number of holes can be adjusted. Selection of a suitable device depends on parameters, such as nature of the drug and its formulation, the site of action, and pathophysiology of the lung. Aqueous suspensions and solutions are nebulized effectively. Aerosols based on mechanically generated vibration mesh technologies also have been used successfully to deliver proteins to lungs.

The amount of RNAi agent for pulmonary system administration may vary from one target gene to another target gene and the appropriate amount that has to be applied may have to be determined individually for each target gene. Typically, this amount ranges from 10 μg to 2 mg, or 50 g to 1500 μg, or 100 g to 1000 μg.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves, and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added. Compositions suitable for oral administration of the agents of the invention are further described in PCT Application No. PCT/US20/33156, the entire contents of which are incorporated herein by reference.

Formulations for parenteral administration may include sterile aqueous solutions which may also contain buffers, diluents, and other suitable additives. For intravenous use, the total concentration of solutes may be controlled to render the preparation isotonic.

In one embodiment, the administration of the siRNA compound, e.g., a double-stranded siRNA compound, is parenteral, e.g., intravenous (e.g., as a bolus or as a diffusible infusion), intradermal, intraperitoneal, intramuscular, subcutaneous, transmucosal, buccal, sublingual, endoscopic, rectal, oral, vaginal, topical, pulmonary system, intranasal, urethral, or ocular. Administration can be provided by the subject or by another person, e.g., a health care provider. The medication can be provided in measured doses or in a dispenser which delivers a metered dose. Selected modes of delivery are discussed in more detail below.

### A. Vector Encoded RNAi Agents of the Disclosure

RNAi agents targeting the target gene can be expressed from transcription units inserted into DNA or RNA vectors (see, e.g., Couture, A, et al., TIG. (1996), 12:5-10; WO 00/22113, WO 00/22114, and U.S. Pat. No. 6,054,299). Expression can be sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of a RNAi agent can be transcribed from a promoter on an expression vector. Where two separate strands are to be expressed to generate, for example, a dsRNA, two separate expression vectors can be co-introduced (e.g., by transfection or infection) into a target cell. Alternatively, each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

RNAi agent expression vectors are generally DNA plasmids or viral vectors. Expression vectors compatible with eukaryotic cells, such as those compatible with vertebrate cells, can be used to produce recombinant constructs for the expression of a RNAi agent as described herein. Delivery of RNAi agent expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of a RNAi agent will generally require regulatory elements, e.g., promoters, enhancers, etc., to ensure the expression of the RNAi agent in target cells. Other aspects to consider for vectors and constructs are known in the art.

### IX. Pharmaceutical Compostions

The present disclosure also includes pharmaceutical compositions and formulations which include the RNAi agents of the disclosure. In one embodiment, provided herein are pharmaceutical compositions containing an RNAi agent, as described herein, and a pharmaceutically acceptable carrier. The pharmaceutical compositions containing the RNAi agent are useful for treating a subject who would benefit from inhibiting or reducing the expression of a target gene, e.g., a subject having a target gene-associated disorder. Such pharmaceutical compositions are formulated based on the mode of delivery. One example is compositions that are formulated for systemic administration via parenteral delivery, e.g., by intravenous (IV), intramuscular (IM), or for subcutaneous (subQ) delivery.

In some embodiments, the pharmaceutical compositions of the invention are pyrogen free or non-pyrogenic.

In one embodiment, the delivery vehicle can deliver an iRNA compound, e.g., a double-stranded iRNA compound, or ssiRNA compound, (e.g., a precursor thereof, e.g., a larger siRNA compound which can be processed into a ssiRNA compound, or a DNA which encodes an siRNA compound, e.g., a double-stranded siRNA compound, or ssiRNA compound, or precursor thereof) to a cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a pharmaceutical composition including an siRNA compound, e.g., a double-stranded siRNA compound, or ssiRNA compound, (e.g., a precursor thereof, e.g., a larger siRNA compound which can be processed into a ssiRNA compound, or a DNA which encodes an siRNA compound, e.g., a double-stranded siRNA compound, or ssiRNA compound, or precursor thereof) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including an siRNA compound, e.g., a double-stranded siRNA compound, or ssiRNA compound, (e.g., a precursor thereof, e.g., a larger siRNA compound which can be processed into a ssiRNA compound, or a DNA which encodes an siRNA compound, e.g., a double-stranded siRNA compound, or ssiRNA compound, or precursor thereof) in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

### X. Methods of the Invention

Another aspect of the invention relates to a method of reducing the expression of a target gene in a cell, comprising contacting the cell with a dsRNA agent of the invention. The methods include contacting the cell with a dsRNA of the disclosure and maintaining the cell for a time sufficient to obtain degradation of the mRNA transcripts of a target gene, thereby inhibiting expression of the target gene in the cell.

Reduction in gene expression can be assessed by any methods known in the art. For example, a reduction in the expression of a target may be determined by determining the mRNA expression level of the target gene using methods routine to one of ordinary skill in the art, e.g., northern blotting, qRT-PCR; by determining the protein level of a target protein using methods routine to one of ordinary skill in the art, such as western blotting, immunological techniques or histology based methods, such as IHC and ISH.

In the methods of the disclosure the cell may be contacted in vitro or in vivo, i.e., the cell may be within a subject. Contacting a cell in vivo with the RNAi agent includes contacting a cell or group of cells within a subject, e.g., a human subject, with the RNAi agent. Combinations of in vitro and in vivo methods of contacting a cell are also possible.

The cell may be an extra-heptic cell, such as askeletal muscle cell, a cardiac muscle cell, or an adipocyte.

A cell suitable for treatment using the methods of the disclosure may be any cell that expresses a target gene. A cell suitable for use in the methods of the disclosure may be a mammalian cell, e.g., a primate cell (such as a human cell or a non-human primate cell, e.g., a monkey cell or a chimpanzee cell), a non-primate cell (such as a rat cell, or a mouse cell. In one embodiment, the cell is a human cell, e.g., a human liver cell or a human kidney cell.

Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc ligand, or any other ligand that directs the RNAi agent to a site of interest. In certain embodiments, the RNAi agent does not include a targeting ligand.

The term “inhibiting,” as used herein, is used interchangeably with “reducing,” “silencing,” “downregulating,” “suppressing” and other similar terms, and includes any level of inhibition. In certain embodiments, a level of inhibition, e.g., for an RNAi agent of the instant disclosure, can be assessed in cell culture conditions, e.g., wherein cells in cell culture are transfected via Lipofectamine™-mediated transfection at a concentration in the vicinity of a cell of 10 nM or less, 1 nM or less, etc. Knockdown of a given RNAi agent can be determined via comparison of pre-treated levels in cell culture versus post-treated levels in cell culture, optionally also comparing against cells treated in parallel with a scrambled or other form of control RNAi agent. Knockdown in cell culture of, e.g., 50% or more, can thereby be identified as indicative of “inhibiting” or “reducing”, “downregulating” or “suppressing”, etc. having occurred. It is expressly contemplated that assessment of targeted mRNA or encoded protein levels (and therefore an extent of “inhibiting”, etc. caused by a RNAi agent of the disclosure) can also be assessed in in vivo systems for the RNAi agents of the instant disclosure, under properly controlled conditions as described in the art.

The phrase “inhibiting expression of a target gene” or “inhibiting expression of a target,” as used herein, includes inhibition of expression of any target gene (such as, e.g., a mouse target gene, a rat target gene, a monkey target gene, or a human target gene) as well as variants or mutants of a target gene that encode a target protein. Thus, the target gene may be a wild-type target gene, a mutant target gene, or a transgenic target gene in the context of a genetically manipulated cell, group of cells, or organism.

“Inhibiting expression of a target gene” includes any level of inhibition of a target gene, e.g., at least partial suppression of the expression of a target gene, such as an inhibition by at least 20%. In certain embodiments, inhibition is by at least 30%, at least 40%, at least 50%, at least about 60%, at least 70%, at least about 80%, at least 85%, at least 90%, at least 95%, at least 96%, at least 97%, at least 98%, or at least 99%; or to below the level of detection of the assay method. In certain method, inhibition is measured at a 10 nM concentration of the siRNA using the luciferase assay provided in Example 1.

The expression of a target gene may be assessed based on the level of any variable associated with target gene expression, e.g., target mRNA level or target protein level.

Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the disclosure, expression of a target gene is inhibited by at least 20%, 30%, 40%, 50%, 60%, 70%, 80%, 85%, 90%, or 95%, or to below the level of detection of the assay. In certain embodiments, the methods include a clinically relevant inhibition of expression of a target gene, e.g. as demonstrated by a clinically relevant outcome after treatment of a subject with an agent to reduce the expression of a target gene.

Inhibition of the expression of a target gene may be manifested by a reduction of the amount of mRNA expressed by a first cell or group of cells (such cells may be present, for example, in a sample derived from a subject) in which a target gene is transcribed and which has or have been treated (e.g., by contacting the cell or cells with a RNAi agent of the disclosure, or by administering a RNAi agent of the disclosure to a subject in which the cells are or were present) such that the expression of a target gene is inhibited, as compared to a second cell or group of cells substantially identical to the first cell or group of cells but which has not or have not been so treated (control cell(s) not treated with a RNAi agent or not treated with a RNAi agent targeted to the genome of interest). The degree of inhibition may be expressed in terms of:

$$\frac{( mRNA in control cells ) - ( mRNA in treated cells )}{( mRNA in control cells )} 100 \%$$

In other embodiments, inhibition of the expression of a target gene may be assessed in terms of a reduction of a parameter that is functionally linked to a target gene expression, e.g., target protein expression. Target gene silencing may be determined in any cell expressing a target gene, either endogenous or heterologous from an expression construct, and by any assay known in the art.

Inhibition of the expression of a target protein may be manifested by a reduction in the level of the target protein that is expressed by a cell or group of cells (e.g., the level of protein expressed in a sample derived from a subject). As explained above, for the assessment of genome suppression, the inhibition of protein expression levels in a treated cell or group of cells may similarly be expressed as a percentage of the level of protein in a control cell or group of cells.

A control cell or group of cells that may be used to assess the inhibition of the expression of a target gene includes a cell or group of cells that has not yet been contacted with an RNAi agent of the disclosure. For example, the control cell or group of cells may be derived from an individual subject (e.g., a human or animal subject) prior to treatment of the subject with an RNAi agent.

The level of target gene mRNA that is expressed by a cell or group of cells may be determined using any method known in the art for assessing RNA expression. In one embodiment, the level of expression of target gene in a sample is determined by detecting a transcribed polynucleotide, or portion thereof, e.g., mRNA of the target gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene (PreAnalytix, Switzerland). Typical assay formats utilizing ribonucleic acid hybridization include nuclear run-on assays, RT-PCR, RNase protection assays, northern blotting, in situ hybridization, microarray analysis, and or histology based methods such as IHC and ISH. Circulating target mRNA may be detected using methods the described in WO2012/177906, the entire contents of which are hereby incorporated herein by reference.

In some embodiments, the level of expression of target gene is determined using a nucleic acid probe. The term “probe”, as used herein, refers to any molecule that is capable of selectively binding to a specific target nucleic acid or protein, or fragment thereof. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of RNA levels involves contacting the isolated RNA with a nucleic acid molecule (probe) that can hybridize to target RNA. In one embodiment, the RNA is immobilized on a solid surface and contacted with a probe, for example by running the isolated RNA on an agarose gel and transferring the RNA from the gel to a membrane, such as nitrocellulose. In an alternative embodiment, the probe(s) are immobilized on a solid surface and the RNA is contacted with the probe(s), for example, in an Affymetrix® gene chip array. A skilled artisan can readily adapt known RNA detection methods for use in determining the level of target mRNA.

An alternative method for determining the level of expression of target in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of target is determined by quantitative fluorogenic RT-PCR (i.e., the TaqMan™ System), by a Dual-Glo® Luciferase assay, or by other art-recognized method for measurement of target expression or mRNA level.

The expression level of target mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of target expression level may also comprise using nucleic acid probes in solution.

In some embodiments, the level of RNA expression is assessed using branched DNA (bDNA) assays or real time PCR (qPCR). The use of this PCR method is described and exemplified in the Examples presented herein. Such methods can also be used for the detection of target nucleic acids.

The level of target protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of target proteins.

In some embodiments, the efficacy of the methods of the disclosure in the treatment of a target gene-related disease is assessed by a decrease in target mRNA level (e.g, by assessment of a blood target gene level, or otherwise).

In some embodiments, the efficacy of the methods of the disclosure in the treatment of a target gene-related disease is assessed by a decrease in target mRNA level (e.g, by assessment of a liver or kidney sample for target level, by biopsy, or otherwise).

In some embodiments of the methods of the disclosure, the RNAi agent is administered to a subject such that the RNAi agent is delivered to a specific site within the subject. The inhibition of expression of target may be assessed using measurements of the level or change in the level of target mRNA or target protein in a sample derived from a specific site within the subject, e.g., liver or kidney cells. In certain embodiments, the methods include a clinically relevant inhibition of expression of target, e.g. as demonstrated by a clinically relevant outcome after treatment of a subject with an agent to reduce the expression of target gene.

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

The in vivo methods of the disclosure may include administering to a subject a composition containing a RNAi agent, where the RNAi agent includes a nucleotide sequence that is complementary to at least a part of an RNA transcript of the target gene of the subject to be treated. When the organism to be treated is a mammal such as a human, the composition can be administered by any means known in the art including, but not limited to oral, intraperitoneal, or parenteral routes, including intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered orally. In certain embodiments, the compositions are administered by pulmonary delivery, e.g., oral inhalation or intranasal delivery.

In some embodiments, the administration is via a depot injection. A depot injection may release the RNAi agent in a consistent way over a prolonged time period. Thus, a depot injection may reduce the frequency of dosing needed to obtain a desired effect, e.g., a desired inhibition of the target gene, or a therapeutic or prophylactic effect. A depot injection may also provide more consistent serum concentrations. Depot injections may include subcutaneous injections or intramuscular injections. In certain embodiments, the depot injection is a subcutaneous injection.

In one embodiment, the double-stranded RNAi agent is administered by pulmonary system administration, e.g., intranasal administration or oral inhalative administration. Pulmonary system administration may be via a syringe, a dropper, atomization, or use of device, e.g., a passive breath driven or active power driven single/-multiple dose dry powder inhaler (DPI) device.

The mode of administration may be chosen based upon whether local or systemic treatment is desired and based upon the area to be treated. The route and site of administration may be chosen to enhance targeting.

In one aspect, the present disclosure also provides methods for inhibiting the expression of a target gene in a mammal. The methods include administering to the mammal a composition comprising a dsRNA that targets a target gene in a cell of the mammal and maintaining the mammal for a time sufficient to obtain degradation of the RNA transcript of the target gene, thereby inhibiting expression of the target gene in the cell. Reduction in genome expression can be assessed by any methods known it the art and by methods, e.g. qRT-PCR, described herein. Reduction in protein production can be assessed by any methods known it the art and by methods, e.g. ELISA, described herein.

The present disclosure further provides methods of treatment of a subject in need thereof. The treatment methods of the disclosure include administering an RNAi agent of the disclosure to a subject, e.g., a subject that would benefit from inhibition of target gene expression, in a therapeutically effective amount of a RNAi agent targeting a target gene or a pharmaceutical composition comprising a RNAi agent targeting a target gene.

Target genes (described above), target gene-associated disorders, and subjects that would benefit from a reduction or inhibition of target gene expression, e.g., those having a target gene-associated disease, subjects at risk of developing a target gene-associate disease, are described below.

An RNAi agent of the disclosure may be administered as a “free RNAi agent.” A free RNAi agent is administered in the absence of a pharmaceutical composition. The naked RNAi agent may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the RNAi agent can be adjusted such that it is suitable for administering to a subject. In certain embodiments, the free RNAi agent may be formulated in water or normal saline.

Alternatively, an RNAi agent of the disclosure may be administered as a pharmaceutical composition, such as a dsRNA liposomal formulation.

In one aspect, the present invention provides a method of treating a subject having a skeletal muscle disorder, a cardiac muscle disorder, or an adipose tissue disorder, comprising administering to the subject a therapeutically effective amount of a dsRNA agent of the invention, thereby treating the subject.

Exemplary cardiac muscle disorders include obstructive hypertrophic cardiomyopathy (HOCM); familial hypertrophic cardiomyopathy (FHC); Heart failure with preserved ejection fraction (HFPEF); atrial fibrillation (AFIB); ventricular fibrillation (VFIB); angina; myocardial infarction (MI); heart failure or heart failure with reduced ejection fraction (HFREF); supraventricular tachycardia (SVT); and hypertrophic cardiomyopathy (HCM).

Exemplary skeletal muscle disorders include Myostatin-related muscle hypertrophy, congenital myasthenic syndrome, and facioscapulohumeral muscular dystrophy (FSHD).

Exemplary adipose tissue disorders include a metabolic disorder, e.g. metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

The dsRNA agent of the invention can be delivered to a subject by a variety of routes, depending on the type of genes targeted and the type of disorders to be treated. In some embodiments, the dsRNA agent is administered extra-hepatically, such as intravenous, intramuscular, or subcutaneous administration.

The disclosure further provides methods for the use of a RNAi agent or a pharmaceutical composition thereof, e.g., for treating a subject that would benefit from reduction or inhibition of target gene expression, e.g., a subject having a target-gene-associated disorder, in combination with other pharmaceuticals or other therapeutic methods, e.g., with known pharmaceuticals or known therapeutic methods, such as, for example, those which are currently employed for treating these disorders.

Examples of the additional therapeutic agents which can be used with an RNAi agent of the invention include, but are not limited to, diabetes mellitus-treating agents, diabetic complication-treating agents, cardiovascular diseases-treating agents, anti-hyperlipemic agents, hypotensive or antihypertensive agents, anti-obesity agents, nonalcoholic steatohepatitis (NASH)-treating agents, chemotherapeutic agents, immunotherapeutic agents, immunosuppressive agents, nonsteroidal anti-inflammatory drugs (NSAIDs), colchicine, corticosteroids, and the like. Such combination therapies may advantageously utilize lower dosages of the administered therapeutic agents, thus avoiding possible toxicities or complications associated with the various monotherapies.

Examples of agents for treating diabetes mellitus include insulin formulations (e.g., animal insulin formulations extracted from a pancreas of a cattle or a swine; a human insulin formulation synthesized by a gene engineering technology using microorganisms or methods), insulin sensitivity enhancing agents, pharmaceutically acceptable salts, hydrates, or solvates thereof (e.g., pioglitazone, troglitazone, rosiglitazone, netoglitazone, balaglitazone, rivoglitazone, tesaglitazar, farglitazar, CLX-0921, R-483, NIP-221, NIP-223, DRF-2189, GW-7282TAK-559, T-131, RG-12525, LY-510929, LY-519818, BMS-298585, DRF-2725, GW-1536, GI-262570, KRP-297, TZD18 (Merck), DRF-2655, and the like), alpha-glycosidase inhibitors (e.g., voglibose, acarbose, miglitol, emiglitate and the like), biguanides (e.g., phenformin, metformin, buformin and the like) or sulfonylureas (e.g., tolbutamide, glibenclamide, gliclazide, chlorpropamide, tolazamide, acetohexamide, glyclopyramide, glimepiride and the like) as well as other insulin secretion-promoting agents (e.g., repaglinide, senaglinide, nateglinide, mitiglinide, GLP-1 and the like), amyrin agonist (e.g., pramlintide and the like), phosphotyrosin phosphatase inhibitor (e.g., vanadic acid and the like) and the like.

Examples of agents for treating diabetic complications include, but are not limited to, aldose reductase inhibitors (e.g., tolrestat, epalrestat, zenarestat, zopolrestat, minalrestat, fidareatat, SK-860, CT-112 and the like), neurotrophic factors (e.g., NGF, NT-3, BDNF and the like), PKC inhibitors (e.g., LY-333531 and the like), advanced glycation end-product (AGE) inhibitors (e.g., ALT946, pimagedine, pyradoxamine, phenacylthiazolium bromide (ALT766) and the like), active oxygen quenching agents (e.g., thioctic acid or derivative thereof, a bioflavonoid including flavones, isoflavones, flavonones, procyanidins, anthocyanidins, pycnogenol, lutein, lycopene, vitamins E, coenzymes Q, and the like), cerebrovascular dilating agents (e.g., tiapride, mexiletene and the like).

Anti-hyperlipemic agents include, for example, statin-based compounds which are cholesterol synthesis inhibitors (e.g., pravastatin, simvastatin, lovastatin, atorvastatin, fluvastatin, rosuvastatin and the like), squalene synthetase inhibitors or fibrate compounds having a triglyceride-lowering effect (e.g., fenofibrate, gemfibrozil, bezafibrate, clofibrate, sinfibrate, clinofibrate and the like), niacin, PCSK9 inhibitors, triglyceride lowing agents or cholesterol sequesting agents.

Hypotensive agents include, for example, angiotensin converting enzyme inhibitors (e.g., captopril, enalapril, delapril, benazepril, cilazapril, enalapril, enalaprilat, fosinopril, lisinopril, moexipril, perindopril, quinapril, ramipril, trandolapril and the like) or angiotensin II antagonists (e.g., losartan, candesartan cilexetil, olmesartan medoxomil, eprosartan, valsartan, telmisartan, irbesartan, tasosartan, pomisartan, ripisartan forasartan, and the like) or calcium channel blockers (e.g., amlodipine) or aspirin.

Nonalcoholic steatohepatitis (NASH)-treating agents include, for example, ursodiol, pioglitazone, orlistat, betaine, rosiglitazone.

Anti-obesity agents include, for example, central antiobesity agents (e.g., dexfenfluramine, fenfluramine, phentermine, sibutramine, amfepramone, dexamphetamine, mazindol, phenylpropanolamine, clobenzorex and the like), gastrointestinal lipase inhibitors (e.g., orlistat and the like), beta 3-adrenoceptor agonists (e.g., CL-316243, SR-58611-A, UL-TG-307, SB-226552, AJ-9677, BMS-196085 and the like), peptide-based appetite-suppressing agents (e.g., leptin, CNTF and the like), cholecystokinin agonists (e.g., lintitript, FPL-15849 and the like) and the like.

In addition, agents whose cachexia improving effect has been established in an animal model or at a clinical stage, such as cyclooxygenase inhibitors (e.g., indomethacin and the like), progesterone derivatives (e.g., megestrol acetate), glucosteroid (e.g., dexamethasone and the like), metoclopramide-based agents, tetrahydrocannabinol-based agents, lipid metabolism improving agents (e.g., eicosapentanoic acid and the like), growth hormones, IGF-1, antibodies against TNF-α, LIF, IL-6 and oncostatin M may also be employed concomitantly with an RNAi agent according to the present invention. Additional therapeutic agents for use in the treatment of diseases or conditions related to metabolic disorders and/or impaired neurological signaling would be apparent to the skilled artisan and are within the scope of this disclosure.

The RNAi agent and additional therapeutic agents may be administered at the same time or in the same combination, or the additional therapeutic agent can be administered as part of a separate composition or at separate times or by another method known in the art or described herein.

In one embodiment, the method includes administering a composition featured herein such that expression of the target gene is decreased, for at least one month. In some embodiments, expression is decreased for at least 2 months, 3 months, or 6 months.

In certain embodiments, administration includes a loading dose administered at a higher frequency, e.g., once per day, twice per week, once per week, for an initial dosing period, e.g., 2-4 doses.

In some embodiments, the RNAi agents useful for the methods and compositions featured herein specifically target RNAs (primary or processed) of the target gene. Compositions and methods for inhibiting the expression of these genes using RNAi agents can be prepared and performed as described herein.

Administration of the dsRNA according to the methods of the disclosure may result in a reduction of the severity, signs, symptoms, or markers of such diseases or disorders in a patient with a target gene-associated disorder. By “reduction” in this context is meant a statistically significant or clinically significant decrease in such level. The reduction can be, for example, at least 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. It is well within the ability of one skilled in the art to monitor efficacy of treatment or prevention by measuring any one of such parameters, or any combination of parameters. In connection with the administration of a RNAi agent targeting a target gene of interest or pharmaceutical composition thereof, “effective against” a target gene-associated disorder indicates that administration in a clinically appropriate manner results in a beneficial effect for at least a statistically significant fraction of patients, such as an improvement of symptoms, a cure, a reduction in disease, extension of life, improvement in quality of life, or other effect generally recognized as positive by medical doctors familiar with treating target gene-associated disorders and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and at least 20%, 30%, 40%, 50%, or more can be indicative of effective treatment. Efficacy for a given RNAi agent drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using a RNAi agent or RNAi agent formulation as described herein.

Subjects can be administered a therapeutic amount of dsRNA, such as about 0.01 mg/kg to about 200 mg/kg.

The RNAi agent can be administered over a period of time, on a regular basis. In certain embodiments, after an initial treatment regimen, the treatments can be administered on a less frequent basis. Administration of the RNAi agent can reduce target gene levels, e.g., in a cell, tissue, blood sample or other compartment of the patient by at least 20%, 30%, 40%, 50%, 55%, 60%, 65%, 70,% 75%, 80%, 85%, 90%, 95%, 96%, 97%, 98%, or at least about 99% or more. In one embodiment, administration of the RNAi agent can reduce target gene levels, e.g., in a cell, tissue, blood sample, or other compartment of the patient by at least 50%.

Before administration of a full dose of the RNAi agent, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Alternatively, the RNAi agent can be administered by oral administration, pulmonary admistration, intravenously, i.e., by intravenous injection, or subcutaneously, i.e., by subcutaneous injection. One or more injections may be used to deliver the desired, e.g., monthly dose of RNAi agent to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of RNAi agent on a regular basis, such as monthly or extending to once a quarter, twice per year, once per year. In certain embodiments, the RNAi agent is administered about once per month to about once per quarter (i.e., about once every three months).

### XI. Target Genes and Target-Gene-Associated Diseases

### Target Genes

Without limitations, genes targeted by the siRNAs of the invention include, but are not limited to genes which mediate a skeletal muscle disorder, a cardiac muscle disorder, or an adipose tissue disorder.

Specific exemplary target genes that mediate a cardiac muscle disorder include, but are not limited to, adrenoceptor beta 1 (ADRB1); calcium voltage-gated channel subunit alpha1 C (CACNA1C); calcium voltage-gated channel subunit alpha1 G (CACNA1G) (T type calcium channel); angiotensin II receptor type 1 (AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); or Phosphodiesterase 1 (PDE1).

Specific exemplary target genes that mediate a skeletal muscle disorder include, but are not limited to, myostatin (MSTN); Cholinergic Receptor Nicotinic Alpha 1 Subunit (CHRNA1); Cholinergic Receptor Nicotinic Beta 1 Subunit (CHRNB1); Cholinergic Receptor Nicotinic Delta Subunit (CHRND); Cholinergic Receptor Nicotinic Epsilon Subunit (CHRNE); Cholinergic Receptor Nicotinic Gamma Subunit (CHRNG); Collagen Type XIII Alpha 1 Chain (COL13A1); Docking Protein 7 (DOK7); LDL Receptor Related Protein 4 (LRP4); Muscle Associated Receptor Tyrosine Kinase (MUSK); Receptor Associated Protein Of The Synapse (RAPSN); Sodium Voltage-Gated Channel Alpha Subunit 4 (SCN4A); and Double Homeobox 4 (DUX4).

Specific exemplary target genes that mediate an adipose tissue disorder include, but are not limited to, Delta 4-Desaturase, Sphingolipid 1 (DEGS1); leptin; folliculin (FLCN); Zinc Finger Protein 423 (ZFP423); Cyclin Dependent Kinase 6 (CDK6); Regulatory Associated Protein Of MTOR Complex 1 (RPTOR); Mechanistic Target Of Rapamycin Kinase, (mTOR); Forkhead Box P1 (FOXP1); Phosphodiesterase 3B (PDE3B); and Activin A Receptor Type 1C (ACVR1C).

### Targeting Cardiac Tissue

In some embodiments, the present invention provides a double-stranded iRNA agent that targets ADRB1 for the treatment of obstructive hypertrophic cardiomyopathy (HOCM); familial hypertrophic cardiomyopathy (FHC); heart failure with preserved ejection fraction (HF-pEF); atrial fibrillation (AFIB); ventricular fibrillation (VFIB); angina; myocardial infarction (MI); and/or heart failure or heart failure with reduced ejection fraction (HFREF).

In some embodiments, the present invention provides a double-stranded iRNA agent that targets CACNA1C for the treatment of supraventricular tachycardia (SVT); AFIB; Angina; and/or HOCM.

In some embodiments, the present invention provides a double-stranded iRNA agent that targets CACNA1G for the treatment of supraventricular tachycardia (SVT); and/or angina.

In some embodiments, the present invention provides a double-stranded iRNA agent that targets AGTR1 for the treatment of HOCM; hypertrophic cardiomyopathy (HCM); and/or HF-pEF.

In some embodiments, the present invention provides a double-stranded iRNA agent that targets SCN2A for the prevention and/or treatment of AFIB.

In some embodiments, the present invention provides a double-stranded iRNA agent that targets HCN1 for the prevention and/or treatment of AFIB; treatment, e.g., rate control, in HOCM.

In some embodiments, the present invention provides a double-stranded iRNA agent that targets HCN4 for the prevention and/or treatment of AFIB; treatment, e.g., rate control, in HOCM.

In some embodiments, the present invention provides a double-stranded iRNA agent that targets HCN3 for the prevention and/or treatment of AFIB; treatment, e.g., rate control, in HOCM.

In some embodiments, the present invention provides a double-stranded iRNA agent that targets KCNA5 for the prevention and/or treatment of AFIB, e.g., AFIB in congestive heart failure (CHF).

In some embodiments, the present invention provides a double-stranded iRNA agent that targets KCNJ3 for the prevention and/or treatment of AFIB.

In some embodiments, the present invention provides a double-stranded iRNA agent that targets KCNJ4 for the prevention and/or treatment of AFIB.

In some embodiments, the present invention provides a double-stranded iRNA agent that targets CAMK2D for the prevention and/or treatment of heart failure and/or AFIB.

In some embodiments, the present invention provides a double-stranded iRNA agent that targets PLN for the prevention and/or treatment of HF-rEF, arrhythmia, and/or cardiomyopathy. In some embodiments, the present invention provides a double-stranded iRNA agent that targets PDE1 for the prevention and/or treatment of CHF and/or HF-pEF.

### Targeting ADRB1 for the Prevention and or Treatment of HOCM, FHC, HFPEF, AFIB, VFIB, Angina, MI, and or HFREF

The beta-adrenergic receptors (ADRBs) are part of a family of membrane proteins known as G-protein coupled receptors where, upon binding of a catecholamine to the receptor, stimulates a conformational change in the ADRB that causes coupling with G-proteins. G-proteins consist of α, β, and γ subunits and ADRB coupling leads to the dissociation of the G-protein into active Ga and GD subunits to mediate downstream signaling.

Beta-adrenergic receptors (ADRBs) play an important role in the extrinsic control of cardiac contractility and function, and are important drug targets for cardiovascular conditions such as hypertension and congestive heart failure. Inhaled beta-receptor (e.g. “beta-blockers”) remain among the mostly commonly prescribed medications in adults to treat cardiovascular disease.

There are three subtypes ofADRBs (ADRB1, ADRB2 and ADRB3). ADRB1s are the predominant subtype expressed in the heart. Multiple ADRB1 have been described and found to be associated with various cardiovascular phenotypes, such as hypertension, heart failure, higher heart rates, or response to beta-blocker therapy. Genetic variants of the ADRB1 have also been shown to modulate the cardiac responses to catecholamine binding. In addition, ADRB1 signaling has also been shown to play an important role in heart failure (HF), where beta-blocking medications are widely used therapeutic agents. Deleterious effects of ADRB1 signaling include apoptosis, myocyte growth, fibroblast hyperplasia, myopathy, fetal gene induction and proarrhythmia (Mann D L, et al. Circulation. 1992; 85(2):790-804). As an adaptive mechanism in HF, cardiac ADRB1s become less responsive, either downregulating or uncoupling from the G protein pathway (Bristow M R, et al. N Engl J Med. 1982; 307(4):205-211). With respect to atrial fibrillation, ADRB1 variant carriers have been reported to have an increased risk of atrial fibrillation, and higher heart rates during atrial fibrillation. ADRB1 polymorphisms are also associated with ventricular fibrillation (VF) in the context of myocardial infarction (MI).

### Targeting CACNA1C for the Prevention and or Treatment of SVT, AFIB, Angina, and or HOCM

Supraventricular tachycardia (SVT) is a heterogeneous category of cardiac arrhythmias characterized by a fast or tachycardiac rhythm that originates above the atrioventricular (AV) node. The prevalence of SVT is 2.25/1000 persons with a female predominance of 2:1 across all age groups (Lee K W, et al., Curr Probl Cardiol 2008; 33:467-546). The most common SVTs include atrioventricular nodal re-entrant tachycardia, atrioventricular re-entrant tachycardia and atrial tachycardia. SVT increases patient morbidity, particularly when symptoms are frequent or incessant, and in a small cohort of patients with atrial fibrillation (AF) and ventricular pre-excitation, it can be life-threatening.

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78). It can be characterized by rapid and irregular atrial depolarizations with a discrete lack of P waves on electrocardiograms. As a result, the blood in the atria remains static and can promote blood clot formation and increase the risk of stroke (Copley D J, et al. AACN Adv Crit Care 2016; 27:120-8). This can cause detrimental symptoms, impair functional status and reduce the quality of life.

Angina represents the most common symptom of ischaemic heart disease, which is a major cause of death and disability worldwide. Nearly 10 million US adults experience stable angina, which occurs when myocardial oxygen supply does not meet demand, resulting in myocardial ischemia. Stable angina is associated with an average annual risk of 3% to 4% for myocardial infarction or death.

Hypertrophic cardiomyopathy is another common heart disorder, usually genetic in origin, that may affect up to 600,000 people in the United States. The disorder, which is characterized by left ventricular hypertrophy, is usually not progressive, but a small subset of patients develop serious complications, such as progressive heart failure, atrial fibrillation, and sudden cardiac death.

Anti-arrhythmic drug therapies, such as calcium channel blockers, are commonly used to treat heart disorders, such as SVT, AFIB, angina and HOCM, by regulating heart function through shaping the action potential and maintaining the rhythm of cardiac contraction.

In particular, calcium channels, such as CACNA1C, play an important role in regulating heart function. CACNA1C encodes for the α-subunit of the CaV1.2 L-type calcium channel (LTCC), which is critical for the plateau phase of the cardiac action potential, cellular excitability, excitation-contraction coupling, and regulation of gene expression. The currently available calcium channel blockers (e.g., dihydropyridines, phenylalkylamines, and benzothiazepines) all act by binding to different sites on CACNA1C and blocking the calcium current.

The CACNA1C calcium channels open and close at specific times to control the flow of calcium ions into cardiomyocytes at each heartbeat. How long the channels are open and closed is regulated to maintain normal heart function. Perturbations of CACNA1C change the structure of calcium channels throughout the body and have been associated with several different cardiac arrhythmia disorders. The altered channels stay open much longer than usual, which allows calcium ions to continue flowing into cells abnormally. The resulting overload of calcium ions within cardiac muscle cells changes the way the heart beats and can cause abnormal heart muscle contraction and arrhythmia.

Increased susceptibility for arrhythmia was observed in patients with gain of function CACNA1C variants under certain conditions (PLoS One. 2014; 9(9): e106982; Splawski I, et al. Cell. 2004 Oct. 1; 119(1):19-31). Specifically, gain of function mutations of CACNA1C revealed a marked reduction in voltage-dependent inactivation. The consequent increase in calcium influx prolongs the cardiac action potential, and thus the QT interval, and can generate early afterdepolarizations capable of triggering cardiac arrhythmias, such as supraventricular tachycardia and atrial fibrillation. Other CACNA1C variants were also shown to be associated with hypertrophic cardiomyopathy, congenital heart defects, and sudden cardiac death.

### Targeting CACNA1G for the Prevention and or Treatment of SVT and or Angina

Supraventricular tachycardia (SVT) is a heterogeneous category of cardiac arrhythmias characterized by a fast or tachycardiac rhythm that originates above the atrioventricular (AV) node. The prevalence of SVT is 2.25/1000 persons with a female predominance of 2:1 across all age groups (Lee K W, et al., Curr Probl Cardiol 2008; 33:467-546). The most common SVTs include atrioventricular nodal re-entrant tachycardia, atrioventricular re-entrant tachycardia and atrial tachycardia. SVT increases patient morbidity, particularly when symptoms are frequent or incessant, and in a small cohort of patients with atrial fibrillation (AF) and ventricular pre-excitation, it can be life-threatening.

Angina represents the most common symptom of ischaemic heart disease, which is a major cause of death and disability worldwide. Nearly 10 million US adults experience stable angina, which occurs when myocardial oxygen supply does not meet demand, resulting in myocardial ischemia. Stable angina is associated with an average annual risk of 3% to 4% for myocardial infarction or death.

Anti-arrhythmic drug therapies, such as calcium channel blockers, are commonly used to treat heart diseases such as SVT and angina by regulating adult heart function through shaping the action potential and maintaining the rhythm of cardiac contraction.

CACNA1G encodes for the subunit of the CaV3,1 T-type calcium channel, which plays a role in the human sinoatrial node and the conduction system. These channels contribute to the heartbeat by influencing pacemaking and the atrioventricular node. Inactivation of CACNA1G significantly slowed the intrinsic in vivo heart rate, prolonged the sinoatrial node recovery time, and slowed pacemaker activity of individual sinoatrial node cells through a reduction of the slope of the diastolic depolarization (Mangoni, et al., Circulation Research 2006, 1422-1430). Thus, selective blockers of CaV3.1 channels hold promise for therapeutic management of the cardiac diseases that require moderate heart rate reduction, such as SVT. The T-type calcium channels also constitute a promising pharmacological target for the treatment of human diseases, such as epilepsy and chronic pain (Birch P J, et al. Drug Discov Today. 2004; 9:410-418).

### Targeting AGTR1 for the Prevention and or Treatment of HOCM, HCM, and or HFpEF

Hypertrophic cardiomyopathy (HCM) is the most common inheritable cardiac disorder with a phenotypic prevalence of 1:500. It is defined by the presence of left ventricular hypertrophy (LVH) in the absence of loading conditions (hypertension, valve disease) sufficient to cause the observed abnormality.

The obstructive HCM (hypertrophic obstructive cardiomyopathy or HOCM) is subtype of HCM. In HOCM, the wall (septum) between the bottom chambers of the heart thickens. The walls of the pumping chamber can also become stiff. The thickened septum may cause a narrowing that can block or reduce the blood flow from the left ventricle to the aorta, which is a condition called “outflow tract obstruction.”

Increased blood pressure causes a concentric pattern of LVH, which may progress to ventricular dilation and heart failure with preserved ejection fraction (HFpEF). Heart failure with preserved ejection fraction (HFpEF) is a clinical syndrome in which patients have symptoms and signs of heart failure as the result of high ventricular filling pressure despite normal or near normal left ventricular ejection fraction (LVEF≥50 percent). At a cellular level, cardiac myocytes in patients with HFpEF are thicker and shorter than normal myocytes, and collagen content is increased. At the organ level, affected individuals may have concentric remodeling with or without hypertrophy. Increases in myocyte stiffness are mediated in part by relative hypophosphorylation of the sarcomeric molecule titin, due to cyclic guanosine monophosphate (cGMP) deficiency thought to arise primarily as a consequence of increased nitroso-oxidative stress induced by comorbid conditions such as obesity, metabolic syndrome and aging. Cellular and tissue characteristics may become more pronounced as the disease progresses.

Genetic variants in the renin-angiotensin-aldosterone system (RAAS) are considered candidates for these modifying effects. The RAAS system contributes to LVH through effects mediated by circulating angiotensin as well as local activation of RAAS in the myocardium. Angiotensin (Ang) I, produced from angiotensinogen (AGT), is converted to Ang II predominantly by angiotensin-converting enzyme (ACE) and possibly by chymase 1 (CMA1). Ang II binds primarily to the Ang II type 1 receptor (AGTR1) to promote cell growth and hypertrophy. It also stimulates aldosterone by aldosterone synthase (CYP11B2) synthesis, thereby increasing the release of aldosterone, which promotes fluid retention and cardiac fibrosis.

Previous studies suggested a role for specific genetic variants in genes encoding components of the RAAS pathway in modulation of the severity of LVH in patients with HCM (Orenes-Piñero E, et al. JRenin Angiotensin Aldosterone Syst 2011; 12: 521-530; Ortlepp J R, et al., Heart 2002; 87: 270-275). In particular, a specific A>C polymorphism of the AGTR1 gene was considered as the pro-LVH allele. Carriers that harbor the pro-LVH allele had greater left ventricular muscle mass and interventricular septum thickness compared to those without the pro-LVH allele (Kolder et al. Eur J Hum Genet. 2012 October; 20(10): 1071-1077). Additional studies further demonstrated that left ventricular mass is associated with the AGTR1 polymorphism, and cardiac hypertrophy was improved by down-regulating AGTR1 (Y. Yang, et al. Exp. Ther. Med., 12 (3) (2016), pp. 1556-1562).

### Targeting SCN2A for the Prevention and or Treatment of AFIB.

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8). This can cause detrimental symptoms, impair functional status and reduce the quality of life.

Atrial fibrillation can cause syncope or a temporary loss of consciousness caused by a fall in blood pressure. There is also a possibility of atrial fibrillation developing secondary to an epileptic seizure in cases of atrial fibrillation and transient loss of consciousness. Epileptic seizures are often associated with changes in cardiac autonomic function.

Sodium voltage-gated channel alpha subunit 2 (SCN2A) is one of the genes most commonly associated with early-onset epilepsy, and has recently been linked to autism spectrum disorder and developmental delay. SCN2A encodes the Nav1.2 subunit of voltage-gated sodium channel in neurons, which is important for action potential initiation and conduction. SCN2A gain-of-function mutations have been identified, and the phenotypes range from benign neonatal or infantile seizures to severe epileptic encephalopathy. SCN2A gene deletion acts as protective genetic modifier of sudden unexpected death in epilepsy (SUDEP) and suggest measures of brain-heart association as potential indices of SUDEP susceptibility (V Mishra et al., Hum Mol Genet. 2017 Jun. 1; 26(11):2091-2103). In addition to epilepsy and developmental delays, other manifestations of SCN2A deletion can include movement disorders such as dystonia, abnormal gait, ADHD, autism, dysautonomia (i.e. problems with heart rate, blood pressure, and temperature regulation), and GI problems such as feeding difficulties or reflux.

### Targeting HCN1 for the Prevention and or Treatment of AFIB; Treatment, e.g., Rate Control, in HOCM.

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8). This can cause detrimental symptoms, impair functional status and reduce the quality of life.

Hypertrophic cardiomyopathy is another common heart disorder, usually genetic in origin, that may affect up to 600,000 people in the United States. The disorder, which is characterized by left ventricular hypertrophy, is usually not progressive, but a small subset of patients develop serious complications, such as progressive heart failure, atrial fibrillation, and sudden cardiac death.

Hyperpolarization-activated cyclic nucleotide-gated (HCN) channels are voltage-gated channels encoded by the HCN1-4 gene family. These channels are primarily expressed in the heart and in the central and peripheral nervous systems. HCN channels conduct K+ and Na+ ions at a ratio of 3:1 to 5:1. They are activated by hyperpolarization of membrane voltage to −50 mV or below, and conduct the hyperpolarization-activated current, termed I_{f} in heart. In the sino-atrial node (SAN), I_{f} plays an essential role in setting the heart rate and mediating its autonomic control.

HCN1 is highly expressed in the SAN and, in addition, non-pacemaking atrial and ventricular cardiomyocytes also express HCN channels, with an increase in I_{f} activity in ventricular myocytes reported in hypertrophy, ischemic cardiomyopathy and heart failure due to re-expression of HCN genes. Studies have shown that I_{f} current density and occurrence is significantly greater in hypertrophic cardiomyocytes and end-stage failing hearts and this is directly related to the arrhythmias

Genetic variants in HCN channels are linked to sinus node dysfunction, atrial fibrillation, ventricular tachycardia, atrio-ventricular block, Brugada syndrome, sudden infant death syndrome, and sudden unexpected death in epilepsy. HCN1 deficient mice display congenital sinus node dysfunction with severely reduced cardiac output.

Several HCN channel blockers including ZD7288, zatebradine, cilobradine and ivabradine are available. The first clinically approved substance from this new class of drugs is ivabradine.

### Targeting HCN4 for the Prevention and or Treatment of AFIB; Treatment, e.g., Rate Control, in HOCM.

Atrial fibrillation (AF) is the most common type of cardiac arrhythmia. The prevalence of AF increases as the population ages (Dang D, et al. JNatl Med Assoc 2002; 94:1036-48). In AF, the upper chambers of the heart do not function correctly as a result of abnormal electrical signalling (Falk R H. N Engl J Med 2001; 344:1067-78). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8). This can cause detrimental symptoms, impair functional status and reduce the quality of life.

Hypertrophic cardiomyopathy is another common heart disorder, usually genetic in origin, that may affect up to 600,000 people in the United States. The disorder, which is characterized by left ventricular hypertrophy, is usually not progressive, but a small subset of patients develop serious complications, such as progressive heart failure, atrial fibrillation, and sudden cardiac death.

Hyperpolarization-activated cyclic nucleotide-gated (HCN) channels are voltage-gated channels encoded by the HCN1-4 gene family. These channels are primarily expressed in the heart and in the central and peripheral nervous systems. HCN channels conduct K+ and Na+ ions at a ratio of 3:1 to 5:1. They are activated by hyperpolarization of membrane voltage to −50 mV or below, and conduct the hyperpolarization-activated current, termed I_{f} in heart. In the sino-atrial node (SAN), I_{f}plays an essential role in setting the heart rate and mediating its autonomic control.

HCN4 constitutes the predominant isoform in the sino-atrial node (SAN) at both transcript and protein level.

Gain of function variants in HCN4 have been shown to cause rhythm abnormalities, including symptomatic or asymptomatic bradycardia ventricular premature beats, tachycardia-bradycardia syndrome and atrial fibrillation (AF), complete atrioventricular (AV) block, long QT syndrome (LQTS) and torsades de pointes.

Drugs that specifically block HCN channels, e.g., ivabradine, slow the diastolic depolarisation of pacemaker cells, hence cardiac rate, with limited adverse cardiovascular side effects. Selective and quantitatively controlled slowing of heart rate provides an important therapeutic advantage in a variety of cardiac conditions.

### Targeting HCN3 for the Prevention and or Treatment of AFIB; Treatment, e.g., Rate Control, in HOCM.

Atrial fibrillation (AF) is the most common type of cardiac arrhythmia. The prevalence of AF increases as the population ages (Dang D, et al. J Natl Med Assoc 2002; 94:1036-48). In AF, the upper chambers of the heart do not function correctly as a result of abnormal electrical signalling (Falk R H. N Engl J Med 2001; 344:1067-78). It can be characterized by rapid and irregular atrial depolarisations with a discrete lack of P waves on electrocardiograms. As a result, the blood in the atria remains static and can promote blood clot formation and increase the risk of stroke (Copley D J, et al. AACN Adv Crit Care 2016; 27:120-8). This can cause detrimental symptoms, impair functional status and reduce the quality of life.

Hypertrophic cardiomyopathy is another common heart disorder, usually genetic in origin, that may affect up to 600,000 people in the United States. The disorder, which is characterized by left ventricular hypertrophy, is usually not progressive, but a small subset of patients develop serious complications, such as progressive heart failure, atrial fibrillation, and sudden cardiac death.

Hyperpolarization-activated cyclic nucleotide-gated (HCN) channels are voltage-gated channels encoded by the HCN1-4 gene family. These channels are primarily expressed in the heart and in the central and peripheral nervous systems. HCN channels conduct K+ and Na+ ions at a ratio of 3:1 to 5:1. They are activated by hyperpolarization of membrane voltage to −50 mV or below, and conduct the hyperpolarization-activated current, termed I_{f} in heart. In the sino-atrial node (SAN), I_{f} plays an essential role in setting the heart rate and mediating its autonomic control.

Although the cardiac expression of HCN3 channels is low, a ventricular phenotype caused by global deletion of HCN3 has been described. Epicardial myocytes of HCN3 knockouts displayed a reduction of I_{f} density by about 30% and a shortening of action potential duration caused by changes during the late repolarization phase. ECG recordings displayed a slight prolongation of the QT interval combined with increased T-wave amplitudes. These alterations were present only at low heart rates.

Thus, HCN3 contributes to the resting membrane potential and acts as a functional antagonist of hyperpolarizing K currents in late repolarization. Lack of this activity leads to a shortening of action potential duration.

### Targeting KCNA5 for the Prevention and or Treatment of AFIB, e.g., AFIB in Congestive Heart Failure (CHF).

Atrial fibrillation (AF) is the most common cardiac rhythm disorder in clinical practice. During the lifetime of men and women aged≥40 years, there is about 25% risk for the development of AF. This arrhythmia may result in irregular ventricular response, tachycardia-mediated cardiomyopathy, heart failure and thromboembolism. AF accounts for nearly one-third of strokes in individuals above 65 years of age, and is also an independent predictor of mortality. AF is often associated with structural heart diseases or systemic disorders, such as hypertension, coronary artery disease, heart failure, rheumatic heart disease, hyperthyroidism and cardiomyopathies. However, in nearly 10-20% of cases, the underlying etiology for AF cannot be identified by routine examination, and such AF is termed ‘idiopathic’.

Heart failure (HF), including CHF and HF-pEF, affects an estimated 30-50 million patients worldwide. Despite recent therapeutic advances, its prevalence is increasing, partly due to a fall in mortality, but also from higher rates of major co-morbidities such as obesity, diabetes, and age. A major factor underlying cardiac dysfunction in HF resides in second messenger signaling defects coupled to 3′,5′-cyclic adenosine and guanosine monophosphate (cAMP, cGMP). Cyclic AMP stimulates protein kinase A (PKA) and exchange protein activated by cAMP (EPAC), acutely enhancing excitation-contraction coupling and sarcomere function. Cyclic GMP acts as a brake on this signaling by activating protein kinase G (PKG). Both cyclic-nucleotides have relevant vascular and fibroblast activity, reducing vessel tone, altering permeability and proliferation, and suppressing fibrosis. They are synthesized by adenylyl or guanylyl cyclases and degraded (hydrolyzed) by phosphodiesterases (PDEs), to provide tissue and cell specific intracellular nano-regulation.

K+ channels are members of a large family of transmembrane proteins that allow K+ to cross biological membranes selectively. Like Ca2+ and Na+ channels, voltage-gated K+ channels undergo conformational changes to open and close a gate in response to membrane depolarization. The K+ channel family is formed by a complex and diverse group of proteins that is known to exist in all three domains of organisms, eubacteria, archaebacteria, and eukaryotes. K+ channels have a wide range of functions that includes setting the resting membrane potential, modulating electrical excitability, and regulating cell volume.

Cardiac potassium channels maintain the rhythmicity of the heartbeat by repolarizing cardiomyocytes such that the electrical and contractile machineries stay in sync. They alternate between opened and closed conformations in response to the voltage difference across the membrane and form functional homotetrameric channels and heterotetrameric channels that contain variable proportions of KCNA1, KCNA2, KCNA4, KCNA5, and possibly other family members as well.

Dominant-negative mutations in KCNA5 have been demonstrated to fail to generate the ultrarapid delayed rectifier current vital for atrial repolarization and exerted an effect on wild-type current.

### KCNJ3 for the Prevention and or Treatment of AFIB.

As the population ages globally, atrial fibrillation (AF or AFIB) is predicted to affect 6-12 million people in the USA by 2050 and 17.9 million in Europe by 2060. Subjects with atrial fibrillation are 5 to 7 times more likely to have a stroke than the general population. Clots can also travel to other parts of the body (kidneys, heart, intestines), and cause other damage. Atrial fibrillation can also decrease the heart's pumping ability. The irregularity can make the heart work less efficiently. In addition, atrial fibrillation that occurs over a long period of time can significantly weaken the heart and lead to heart failure.

Sinus node cells, located in the right atrium, spontaneously produce an electric impulse (i.e., action potential) that propagates along the cardiac conduction system and causes contraction of the heart muscle. Thus, heart rate is precisely regulated within the proper range by both intrinsic and extrinsic mechanisms. The acetylcholine-activated potassium channel (I_{KACh} channel) expressed in the sinus node, atrium, and atrioventricular node contributes to heart rate slowing triggered by the parasympathetic nervous system. The IKACh channel is a heterotetramer of 2 inwardly rectifying potassium channel proteins, Kir3.1 and Kir3.4, encoded by the genes KCNJ3 and KCNJ5, respectively. As indicated above, KCNJ5 mutation has been associated with atrial fibrillation (AF). However, the molecular basis of IKACh channel pathology remains poorly understood and, to date, rare mutations showing a large effect have not been reported for cardiac diseases.

Autosomal dominant mutations in KCNJ3 have been associated with symptomatic sinus bradycardia, and chronic AF with slow ventricular response. Because I_{KACh} channels are expressed more abundantly in the atrium than in the ventricle, the blockage of IKACh is a target for atrial-selective AF therapy with a lower risk of ventricular arrhythmia.

### Targeting KCNJ4 for the Prevention and or Treatment of AFIB

As the population ages globally, atrial fibrillation (AF or AFIB) is predicted to affect 6-12 million people in the USA by 2050 and 17.9 million in Europe by 2060. Subjects with atrial fibrillation are 5 to 7 times more likely to have a stroke than the general population. Clots can also travel to other parts of the body (kidneys, heart, intestines), and cause other damage. Atrial fibrillation can also decrease the heart's pumping ability. The irregularity can make the heart work less efficiently. In addition, atrial fibrillation that occurs over a long period of time can significantly weaken the heart and lead to heart failure.

Potassium channels, such as KCNJ4, play an important role in regulating adult heart function through shaping the action potential and maintaining the rhythm of cardiac contraction.

KCNJ4 has been identified as a target for antiarrhythmic drugs, as it is expressed at ˜10-fold greater levels in the atria relative to the ventricles of animal model and expression of KCNJ4 Has been shown to be upregulated in left atrial appendage tissues from subjects having AFIB. In addition, gain-of-function mutations of KCNJ4 have been associated with familial forms of AF, and KCNJ4 upregulation contributes to the stabilization/perpetuation of AFIB.

### Targeting PLN for the Prevention and or Treatment of HF-rEF, Arrhythmia, and or Cardiomyopathy

Heart failure, such as heart failure with reduced ejection fraction (HF-rEF), is a major cause of death and disability. The hallmarks of heart failure are impaired cardiac contraction and relaxation accompanied by abnormalities in calcium handling and β-adrenergic signaling (Lou, Q, et al. Adv Exp Med Biol. 2012; 740:1145-1174). In cardiomyocytes, cytosolic calcium regulates cardiac contraction and relaxation by an excitation-contraction coupling mechanism. The Ca2+ influx via L-type calcium channels elicits Ca2+-induced Ca2+ release from sarco(endo)plasmic reticulum (SR) through ryanodine receptors, and increased cytosolic Ca2+ leads to cardiac contraction. The sequestration of Ca2+ from the cytosol into the SR, which determines active relaxation, is caused by a calcium pump on the SR called SR Ca2+ ATPase (SERCA2a), the activity of which is regulated by a small phosphoprotein, phospholamban (PLN) (Kranias, E G, et al., Circ Res. 2012; 110(12):1646-1660).

In physiological conditions, β-adrenergic receptor (PAR) stimulation enhances myocyte contraction by activating cyclic adenosine monophosphate-dependent kinase (protein kinase A [PKA]), which phosphorylates multiple Ca2+ cycling proteins, including PLN. Phospholamban inhibits SERCA2a activity through protein-protein interaction. Phosphorylation of PLN by PKA alters its interaction with SERCA2a to activate Ca2+ reuptake to the SR, resulting in enhanced SR Ca2+ loading and Ca2+ cycling. In the failing myocyte, dysfunctional PAR signaling leads to less PKA activation and activation of alternate pathways, such as calcium/calmodulin-dependent kinase II signaling to cause pathological hypertrophy. Consequently, the usefulness of positive inotropic agents in HF is strongly limited, and direct activation of Ca2+ cycling, which can circumvent dysfunctional PAR activity, is required. Thus, inhibition of PLN is one of the most promising strategies in this context. Several reports have demonstrated that PLN inhibition alleviates cardiac failure in various animal models of cardiac pathologies, including myocardial infarction in rats, genetic cardiomyopathy in hamsters, and dilated cardiomyopathy in mice (Iwanaga, Y, et al., J Clin Invest. 2004; 113(5):727-736; Hoshijima, M, et al., NatMed. 2002; 8(8):864-871; Minamisawa, S. et al., Cell. 1999; 99(3):313-322). In addition, modulation of PLN improves contractility in human cardiomyocytes from patients with advanced HF (del Monte, F, et al. Circulation. 2002; 105(8):904-907), suggesting that targeting PLN is a bona fide therapy for failing hearts.

In particular, the ablation of PLN in mice prevents SERCA2a inhibition and enhances cardiac contractility by increasing the SR Ca2+ store. The ablation of PLN also reverses heart failure in some cardiomyopathic animal models, indicating the possibility of therapeutic approaches. The overexpression of PLN in mouse heart depresses cardiac function and proves that only ˜40% of SERCA pumps are normally regulated by PLN in mouse heart. The superinhibition of SERCA by specific PLN mutants impairs cardiac function and leads to cardiac remodelling and early death if the effects of the mutation cannot be reversed by β-agonists. In human and animal models of heart failure, the PLN-SERCA inhibited complex increases. Interventions that diminish the PLN-SERCA complex have been beneficial in some mouse models of heart failure (MacLennan and Kranias. 2003. Nat Rev Mol Cell Biol 4:566-77).

Dilated cardiomyopathy (DCM) is the second most common cause of heart failure with reduced ejection fraction (HFrEF) after coronary artery disease. It has been estimated that up to 40% of DCM cases have a genetic cause. The p.(Arg14del) pathogenic variant of the PLN gene (PLN-R14del) is a Dutch founder mutation with a high prevalence in DCM and arrhythmogenic cardiomyopathy (ACM) patients. Cardiomyopathy caused by the p.(Arg14del) pathogenic variant of the PLN gene is characterized by intracardiomyocyte PLN aggregation and can lead to severe DCM. Depletion of PLN attenuated heart failure in several cardiomyopathy models. Specifically, PLN knockdown was shown to reduce protein aggregation, normalize autophagy markers, improve cardiomyopathy and survival (Eijgenraam et al. 2022. IntJMol Sci 23:2427. 4). PLN knockdown also reversed the heart failure phenotype in a genetic dilated cardiomyopathy mouse model, and prevented progression of left ventricular dilatation and improveed left ventricular contractility in rats with myocardial infarction (Grote Beverborg et al. 2021. Nat Comm 12:5180). PLN abalation was also shown to reduce susceptibility to ventricular arrhythmias in mouse model of catecholaminergic polymorphic ventricular tachycardia (Mazzocchi et al. 2016 J Physiol 594: 3005-3030).

Thus, inhibition of PLN is an effective strategy in treating and/or preventing genetic cardiomyopathy, arrhythmia, as well as heart failure, in particular HF with reduced ejection fraction (HF-rEF).

### Targeting CAMK2D for the Prevention and or Treatment of Heart Failure and or AFIB

CAMK2D has been shown to associate with the development of cardiac disease, such as heart failure, and arrhythmias (Maier and Bers, 2002, J. Mol. Cell. Cardiol. 34, 919-939; Swaminathan et al., 2012, Circ. Res. 110, 1661-1677). Animal models have shown proof-of-concept studies that transgenic overexpression of CAMK2D is sufficient to induce structural and electrical remodeling in the heart, leading to compromised contractility and increased risk for sudden cardiac death (Zhang et al., 2002, J. Biol. Chem. 277, 1261-1267; Wagner et al., 2011, Circ. Res. 108, 555-565). Likewise, genetic and chemical inhibition of CAMK2D has been shown to confer protection from the development of dilated cardiomyopathy and sustained contractile performance, following both pressure overload and ischemic stress (Backs et al., 2009, J Clin. Invest. 116, 1853-1864; Ling et al., 2009, J Clin. Invest. 119, 1230-1240). Human heart failure has also been associated with an increased expression/activity of CAMK2D (Hoch et al., 1999, Circ. Res. 84, 713-721). The central role for CAMK2D in development of disease stems from its regulation of proteins involved in critical cell functions such Ca2+ cycling. CAMK2D has been implicated in pathologic phosphorylation of a number of Ca2+ handling proteins including phospholamban, leading to activation of the sarcoplasmic reticulum (SR) ATP-driven Ca2+ pump SERCA2a (Mattiazzi and Kranias, 2014, Front. Pharmacol. 5, 5); the ryanodine receptor SR Ca2+ release channel (RyR2) (Witcher et al., 1991, J. Biol. Chem. 266, 11144-11152), promoting increased channel open probability and SR Ca2+ leak; and the L-type Ca2+ channel Cav1.2 and associated β-subunits, potentiating current amplitude and slowing inactivation (Hudmon et al., 2005, J. Cell Biol. 171, 537-547). Collectively, these events not only promote activation of hypertrophic remodeling cascades but also heighten the risk for inappropriate membrane potential depolarizations (afterdepolarizations) that serve as arrhythmia triggers (Wu et al., 2002, Circulation 106, 1288-1293).

In addition to association with heart failure, CAMK2D has also been found to be a GWAS locus for atrial fibrillation (Roselli C et al. 2018. Nat Genet; 50:1225-1233; Ramirez J et al. 2020. Am JHum Genet 106:764-78). Pathological activation of CAMK2D promotes arrhythmia and heart failure (Veitch C R et al. 2021 Front Pharmacol 12: 695401; Nassal D et al. 2020. Front Pharmacol 11:35). In humans, CAMK2D levels and activity are increased in atrial fibrillation and heart failure. In animal models, sustained CAMK2D activation induces adverse structural and electrical remodeling of the heart via phosphorylation of target proteins. Pharmacological and genetic inhibition were shown to prevent these changes. Transagenic expression in the atria of a CAMK2D inhibitory peptide was shown to prevent adverse atrial structure and electrical remodeling (Liu Z et al. 2019. Heart Rhythm 16:1080-1088). In addition, CAMK2D knockout protects against pathological cardiac hypertrophy in a mouse model of heart failure (Backs J et al. PNAS 2009; 106:7:2342-2347).

Thus, suppressing CAMK2D expression is an effective strategy in treating and/or preventing heart failure and/or atrial fibrillation.

### Targeting PDE1 for the Prevention and or Treatment of CHF and or HF-pEF

Heart failure (HF), including CHF and HF-pEF, affects an estimated 30-50 million patients worldwide. Despite recent therapeutic advances, its prevalence is increasing, partly due to a fall in mortality, but also from higher rates of major co-morbidities such as obesity, diabetes, and age. A major factor underlying cardiac dysfunction in HF resides in second messenger signaling defects coupled to 3′,5′-cyclic adenosine and guanosine monophosphate (cAMP, cGMP). Cyclic AMP stimulates protein kinase A (PKA) and exchange protein activated by cAMP (EPAC), acutely enhancing excitation-contraction coupling and sarcomere function. Cyclic GMP acts as a brake on this signaling by activating protein kinase G (PKG). Both cyclic-nucleotides have relevant vascular and fibroblast activity, reducing vessel tone, altering permeability and proliferation, and suppressing fibrosis. They are synthesized by adenylyl or guanylyl cyclases and degraded (hydrolyzed) by phosphodiesterases (PDEs), to provide tissue and cell specific intracellular nano-regulation.

PDE1 is constitutively and robustly expressed in the heart. It is activated by a Ca2+/calmodulin-binding domain and provides a substantial percent of in vitro cAMP and cGMP hydrolytic activity in mammals, including humans.

It has been shown that inhibition of PDE1 prevents phenylephrine-induced myocyte hypertrophy in neonatal and adult rat ventricular myocytes reduces angiotensin II or TGF-induced activation of rat cardiac fibroblasts, and attenuates isoproterenol-induced interstitial fibrosis in mice. Cellular senescence in vascular smooth muscle myocytes leads to elevated PDE1 expression, and PDE1 inhibition restores vasodilatory responses to sodium nitroprusside in aging mice. PDE1 expression in vascular smooth muscle cells in vitro increases with the transition from the contractile to the synthetic phenotype, and PDE1 inhibition attenuates proliferation and migration of vascular smooth muscle cells in culture. PDE1 expression is increased in mouse vascular injury models in vivo and in neointimal smooth muscle cells of human coronary arteries, and injury-induced neointimal formation is reduced by PDE1 inhibition in coronary arteries of mice. Knockout of the PDE1C gene has antihypertrophic, antifibrotic, and antiapoptotic actions in mouse hearts. These observations suggest that PDE1 is a therapeutic target for cardiovascular disease. Indeed, recently a selective small molecule PDE1 inhibitor, ITI-214, was demonstrated to improve cardiac output by increasing heart contractility and decreasing vascular resistance in a Phase I/II study of heart failure patients.

### Targeting Skeletal Muscle Tissue

In some embodiment, the present invention provides a double-stranded iRNA agent that targets myostatin for the treatment of Myostatin-related muscle dystrophy.

In some embodiments, the present invention provides a double-stranded iRNA agent that targets CHRNA1 for the treatment of congenital myasthenic syndrome (CMS).

In some embodiments, the present invention provides a double-stranded iRNA agent that targets CHRNB1 for the treatment of congenital myasthenic syndrome (CMS).

In some embodiments, the present invention provides a double-stranded iRNA agent that targets CHRBD for the treatment of congenital myasthenic syndrome (CMS).

In some embodiments, the present invention provides a double-stranded iRNA agent that targets CHRNE for the treatment of congenital myasthenic syndrome (CMS).

In some embodiments, the present invention provides a double-stranded iRNA agent that targets CHRNG for the treatment of congenital myasthenic syndrome (CMS).

In some embodiments, the present invention provides a double-stranded iRNA agent that targets COL13A1 for the treatment of congenital myasthenic syndrome (CMS).

In some embodiments, the present invention provides a double-stranded iRNA agent that targets LRP4 for the treatment of congenital myasthenic syndrome (CMS).

In some embodiments, the present invention provides a double-stranded iRNA agent that targets MUSK for the treatment of congenital myasthenic syndrome (CMS).

In some embodiments, the present invention provides a double-stranded iRNA agent that targets RAPSN for the treatment of congenital myasthenic syndrome.

In some embodiments, the present invention provides a double-stranded iRNA agent that targets SCN4A for the treatment of congenital myasthenic syndrome (CMS).

In some embodiments, the present invention provides a double-stranded iRNA agent that targets DOK7 for the treatment of congenital myasthenic syndrome (CMS).

In some embodiments, the present invention provides a double-stranded iRNA agent that targets DUX4 for the treatment of Facioscapulohumeral muscular dystrophy (FSHD).

### Targeting Myostatin for the Prevention and or Treatment of Myostatin-Related Muscle Dystrophy

Myostatin, also known as growth differentiation factor 8 (GDF8), is a negative regulator of muscle mass and a member of the TGF-0 superfamily of proteins. Myostatin is initially synthesized by myocytes as a pre-promyostatin molecule composed of an N-terminal signal sequence (for secretion), an N-prodomain region (essential for proper folding of myostatin and subsequently proteolytically processed), and the biologically active C-terminal domain. The precursor pre-promyostatin must undergo proteolytic cleavage to form the biologically active myostatin molecule, which exists as a disulfide-linked dimer of two C-terminal domains. The cleaved propeptide domain also plays a regulatory role through non-covalent binding to the active myostatin C-terminal domain to form an inactive latent myostatin complex. Myostatin is also capable of effecting a non-canonical signaling cascade involving the cellular energy-sensing enzyme AMP-activated kinase (AMPK) and a regulatory protein kinase transforming growth factor-β-activated kinase 1.

Genetic deletion of myostatin has been associated with increasing muscle mass in mice, cattle, dogs, horses, and other species, indicating its evolutionary conservation (McPherron A C, et al., Nature 1997; 387:8390). Discovery of a hypermuscular child who was homozygous for an splice site mutation, which resulted in a premature stop codon, suggested that inhibition of myostatin might confer therapeutic benefits for muscle wasting disease in humans (Schuelke M, et al. New Engl J Med 2004; 350:26822688).

Various myostatin inhibitors have been developed and evaluated as potential treatments for different types of muscular dystrophy. These inhibitors have been shown to ameliorate the phenotype of muscular dystrophy, e.g., by improving muscle mass and strength.

### Targeting CHRNA1, CHRNB1, CHRND, CHRNE and CHRNG for the Prevention and or Treatment of CMS

Congenital myasthenic syndromes (CMS) are a heterogeneous group of rare inherited neuromuscular disorders characterized by fatigable weakness of skeletal muscle owing to compromised function of the neuromuscular junction (NMJ). The phenotype is caused by failure of transmission across this synapse connecting the nerve with the muscle, whereby an incoming nerve stimulus does not consistently lead to muscle excitation and contraction. Neuromuscular transmission is mediated by the generation of an action potential causing the release of acetylcholine from the nerve terminal into the synaptic cleft, its binding to the acetylcholine receptor (AChR) with the opening of its ion channel and the enzymatic breakdown of acetylcholine by acetylcholinesterase (AChE). The AChR controls electrical signalling between nerve and muscle cells by opening and closing a gate, membrane-spanning pore to trigger muscle contraction. It has five subunits of four different types: two alpha and one each of beta, gamma (or epsilon), and delta subunits ((i.e., CHRNE, CHRNA1, CHRNB1, CHRND, and CHRNG). Mutations affecting subunits of the AChR pore cause CMS in humans.

Pathophysiological mechanisms acting on any part of this chain and resulting in a reduction in the amount of acetylcholine released, the impairment of the AChR, reduction in the number of receptors or defective breakdown of acetylcholine may lead to CMS. The majority of CMS types are caused by defects in the AChR itself, but they can also result from causative variants affecting presynaptic proteins or proteins associated with the synaptic basal lamina or variants causing defects in endplate development and maintenance or defects in protein glycosylation. Defective neuromuscular transmission presents clinically as fatigable weakness due to increasing impairment of transmission across the NMJ with repeated activation.

Generalized and fatigable skeletal muscle weakness is the most common clinical sign of CMS, but locus and allelic heterogeneity determine variable severity and additional symptoms. CMS can result from recessive missense, non-sense, or splice site and promoter region mutations in any of the AChR subunits, but most occur in the gamma (or epsilon) subunit.

Diagnosis of CMS is established with clinical and electrodiagnostic features and identification of a causative mutation. In some instances, a clinical diagnosis can be made without finding a causative gene (e.g., individuals who exhibit fatigable weakness, especially of ocular and other cranial muscles, at birth or early childhood). Clinical diagnosis may rely on history, clinical exams, blood tests, incremental or decremental responses or abnormal single-fiber EMG (SF-EMG) study results, lung function tests, polysomnography, the Tensilon test, and muscle biopsy. In rarer cases when symptoms manifest in adolescence or adulthood, symptom presentation may differ from that seen in infants and young children and can include proximal and axial muscle weakness associated with a decremental response requiring prolonged stimulation.

Mutations in about 32 genes that encode proteins involved in this signaling pathway are known to cause CMS. Eight proteins are associated with presynaptic CMS, four with synaptic CMS, fifteen with post-synaptic CMS, and five with glycosylation defects. Proteins affected in CMS have different functions, such as ion channels (AchR), structural proteins (COL13A1, RAPSN), signalling molecules (LRP4, MUSK, DOK7), catalytic enzymes, sensor proteins, or transport proteins. Various gene mutations in presynaptic, synaptic, and postsynaptic proteins have been demonstrated in patients, with more than 50% of the mutations involving aberrations in postsynaptic AChR subunits (i.e., CHRNE, CHRNA1, CHRNB1, CHRND, and CHRNG). Mutations in RAPSN, COLQ, and DOK7 comprise another 35% to 50% of cases.

The CHRNA1 gene encodes the alpha-subunit of the nicotinergic, post-synaptic AchR. CHRNA1 mRNA undergoes alternative splicing and two splice variants (P3A- and P3A+) are produced. Mutations in CHRNA1 result in imbalance between the two splice variants with an increase in P3A+. CHRNA1 mutations reduce the number of AchR at the post-synaptic membrane. The pattern of inheritance is autosomal dominant if CHRNA1 mutations cause a slow channel CMS (SCCMS), or autosomal recessive in case of primary AchR-deficiency. The first CHRNA1-related CMS were reported in 2008. Patients presented already prenatally with growth retardation, reduced movements, edema, contractures, and postnatally with dysmorphism, muscle wasting, scoliosis, contractures, and pterygia. Antisense oligonucleotides (AONs) have been shown to restore the balance between the two splice variants and are thus expected to be beneficial in patients carrying such mutations.

The CHRNB1 gene encodes for the beta-subunit of the nicotinergic, post-synaptic acetylcholine receptor (AChR). Non-synonymous mutations in the human CHRNB1 gene encoding the cholinergic receptor nicotinic beta 1 subunit are known to cause dominant and recessive forms of CMS. The first mutations in CHRNB1 causing CMS were reported in a Brazilian study in 2008. The first patient published was a 28 year old male manifesting since birth with ptosis, ophthalmoparesis, dysphagia, proximal limb muscle weakness, scapular winging, weakness of axial muscles, wasting, and scoliosis. He showed a decremental response to RNS, had double discharges, and a myopathic EMG. The course was progressive but he benefitted from fluoxetine (Mihaylova V, et al. J Neurol Neurosurg Psychiatry. 2010; 81:973-977). The second patient carrying a CHRNB1 mutation was a 3wo male manifesting with ptosis, facial weakness, severe hypotonia, and respiratory insufficiency requiring assisted ventilation (Shen X M, et al., Hum Mutat. 2016; 37:1051-1059). The response to LF-RNS was decremental. In a Spanish study of a CMS cohort, a third patient with a CHRNB1 mutation was identified but no clinical details were provided (atera-de Benito D, et al., Neuromuscul Disord 2017. pii: S0960-8966(17)30475-3).

The CHRND gene encodes the delta-subunit of the nicotinergic, post-synaptic AchR. The first mutation in CHRND causing CMS was reported in a German patient with early-onset CMS manifesting with feeding difficulties, moderate, generalised weakness, and recurrent episodes of respiratory insufficiency provoked by infections (Müller J S, et al., Brain. 2006; 129:2784-2793). The second patient was a 20 year old female with moderate to severe myasthenic manifestations since birth (Shen X M, et al., J Clin Invest. 2008 May; 118(5):1867-76). She had a marked decremental response to LF-RNS. One of her siblings with a similar presentation had died at age 11 m. Two further patients were reported in a study of CMS patients from Israel but no clinical details were provided (Aharoni S, et al., Neuromuscul Disord. 2017 February; 27(2):136-140).

The CHRNE gene encodes for the epsilon-subunit of the AchR. The first mutation in the CHRNE gene causing a CMS has been reported already in 2000 (Sieb J P, et al., Hum Genet. 2000; 107:160-164). Since then various different types of mutations have been reported and it is estimated that up to half of the patients with a CMS carry a CHRNE mutation, thus representing the gene most frequently mutated in CMS. In a study of 64 CMS patients from Spain, CHRNE mutations were detected in 27% of the patients (Natera-de Benito D, et al., Neuromuscul Disord 2017. pii: S0960-8966(17)30475-3). In a study of 45 patients from 35 Israeli CMS families, CHRNE mutations were found in 7 kinships (Aharoni S, et al., Neuromuscul Disord. 2017 February; 27(2):136-140). In a study of 23 families with CMS from Maghreb countries, the founder mutation c.1293insG was found in 60% of these patients (Richard P, et al., Neurology. 2008 Dec. 9; 71(24):1967-72). Type and severity of clinical manifestations of CHRNE mutations may vary considerably between affected families. Some patients may present with only ptosis whereas others may present with severe generalised myasthenia. Most patients present at birth with mildly progressive bulbar, respiratory, or generalized limb weakness with ptosis or ophthalmoplegia. Single patients may die prematurely in infancy from respiratory failure. Some patients may have myasthenic symptoms since birth and achieve ambulation late or not at all. Single patients present with a fluctuating course. Single patients develop severe scoliosis. RNS may be decremental or may be normal. Single-fiber EMG (SF-EMG) may reveal an increased jitter. Some patients may show repetitive CMAPs. Most patients respond favourably to AchE inhibitors.

The CHRNG gene encodes for the fetal gamma-subunit of the AchR. Mutations in the CHRNG gene cause CMS with multiple ptyerygia (lethal multiple pterygia syndrome (LMPS) or the Escobar variant of multiple pterygia syndrome (EVMPS)) (Hoffmann K, et al., Am JHum Genet. 2006; 79:303-312). In a study of seven families with Escobar syndrome (contractions, multiple pterygia, respiratory distress), mutations in the CHRNG gene were detected in 12 family members. The female to male ratio was 7:5. Some patients presented with decreased fetal movements, facial weakness, respiratory distress, arthrogryposis, short stature, kyphosis/scoliosis, dysmorphism, high-arched palate, cleft palate, arachnodactyly, or cryptorchism. None presented with myasthenic manifestations postnatally. CHRNG mutations may be also responsible for the allelic disease fetal akinesia deformation sequence (FADS). In a study of 46 CMS patients from Spain, five carried a mutation in the CHRNG gene (Natera-de Benito D, et al., Neuromuscul Disord 2017. pii: S0960-8966(17)30475-3). They all presented with arthrogryposis and delayed motor milestones, and some of them with poor sucking. Interestingly, none of them received drugs usually given for CMS. In a study of three Iranian CHRNG-related CMS patients, no drug treatment was applied. One of the patients presented with short neck, mild axillar pterygia, elbows and knees, joint contractures, clenched hands with thumbs held across palm and club feet (varus). The patient had rockerbottom feet, with almost no movement in ankles. Facial dysmorphism included hemangioma over forehead and nose, strabismus, flat nasal bridge, and downturned corners of mouth (Kariminejad A, et al., BMC Genet. 2016 May 31; 17(1):71).

Overall, CMS can result from missense, non-sense, or splice site and promoter region mutations in any of the AChR subunits, but most occur in the gamma (or epsilon) subunit. The high frequency of mutations in the epsilon subunit compared with other subunits has been attributed to phenotypic rescue by substitution of the fetal gamma subunit for the defective epsilon subunit (Ohno K, et al. Hum Mol Genet. 1997; 6:753-66). Individuals harboring null mutations in both alleles of CHRNA1, CHRNB1, or CHRND cannot survive because no substituting sub-units exist and hence these individuals probably die in utero (Engel A G, et al., Lancet Neurol. 2015; 14:420-34). Patients with heterozygous or homozygous low-expressor mutations in the non-epsilon subunit are severely affected have high mortality in infancy or early childhood. Thus, given the importance of AchR in CMS, manipulation of AchR subunits are expected to ameliorate the CMS phenotype in patients.

### Targeting COL13A1 for the Prevention and or Treatment of CMS

Mutations in gene encoding synaptic proteins can cause CMS. Collegan XIII is a non-fibrillar transmembrane collagen which has been long recognized for its critical role in synaptic maturation of the neuromuscular junction. The COL13A1 gene encodes the α-chain of collagen XIII with a single transmembrane domain. COL13A1 is localised to the NMJ, where it is responsible for clustering of the AchR during myotube differentiation. Unlike most of the collagens, COL13A1 is anchored to the plasma membrane by a hydrophobic transmembrane segment. The presence of a proprotease recognition site in the ectodomain allows the C-terminus to be proteolytically cleaved into a soluble form that is part of the basal lamina.

Mutations in this gene manifest clinically as CMS, which has been reported in three patients from two families (Logan C V, et al. Am JHum Genet. 2015; 97:878-85). Two of these patients manifested with congenital respiratory insufficiency, bulbar weakness, or facial weakness. All three patients presented with feeding difficulties, ptosis, limb weakness, and dysmorphism. Two patients each presented with spinal stiffness or distal joint laxity, and one patient with ophthalmoparesis and cognitive impairment. Two showed a decremental response to RNS and two an increased jitter. Two required non-invasive positive pressure ventilation.

COL13A1 loss-of-function mutations were also identified in six additional CMS patients from three unrelated families (Dusl M. et al., Journal ofNeurology, 2019; 255: 1107-1112). The phenotype of these cases was similar to the previously reported patients including respiratory distress and severe dysphagia at birth that often resolved or improved in the first days or weeks of life. All individuals had prominent eyelid ptosis with only minor ophthalmoparesis as well as generalized muscle weakness, predominantly affecting facial, bulbar, respiratory and axial muscles. Response to acetylcholinesterase inhibitor treatment was generally negative while salbutamol proved beneficial. These data further support the causality of COL13A1 variants for CMS and suggest that this type of CMS might be clinically homogenous and requires alternative pharmacological therapy.

### Targeting LRP4 for the Prevention and or Treatment of CMS

Some CMS are due to mutations in genes encoding post-synatic proteins. Post-synaptic CMSs represent the vast majority of the CMS subtypes. Post-synaptic CMS are subdivided into primary AchR deficiency, kinetic abnormalities of the AChR, and defects within the AChR-clustering pathway. Mutations in LRP4 cause defects within the AChR-clustering pathway.

The LRP4 gene encodes for lipoprotein receptor-related protein 4, which functions as a receptor for agrin. Agrin, which is released from motor nerve terminals, binds to LRP4 in muscle, stimulating the formation of a complex between LRP4 and muscle-specific kinase (MUSK), a receptor tyrosine kinase that acts as a master regulator of synaptic differentiation. LRP4, once clustered in the postsynaptic membrane as a consequence of MUSK activation, also signals directly back to motor axons to stimulate presynaptic differentiation. Activated MUSK together with DOK7 stimulates rapsyn to concentrate and anchor AchR at the post-synaptic membrane and interacts with other proteins implicated in the assembly and maintenance of the NMJ. LRP4 is thus essential for pre- and post-synaptic specialisation of the NMJ.

The first mutation in the LRP4 gene causing CMS was reported in 2014. A newborn female presented with respiratory arrest and feeding difficulties, and required feeding and ventilator support until 6 m of age. Motor milestones were delayed and she developed easy fatigability with temporary wheelchair-dependency. At ages 9 and 14y she presented with ptosis, ophthalmoparesis, and limb weakness. RNS evoked a decremental response, which improved upon application of edrophonium. AchE inhibitors worsened the clinical manifestations. A second kinship harbouring LRP4 mutations was reported in 2015. The two sisters, aged 34 and 20y, presented with delayed motor milestones, slight chewing and swallowing difficulties, and later developed limb weakness. Albuterol was highly effective.

### Targeting MUSK for the Prevention and or Treatment of CMS

Mutations in MUSCK cause defects within the AChR-clustering pathway. MUSK encodes for a protein that is involved in endplate maturation, maintenance of the endplate functions, proper functioning of rapsyn, and functioning of the AchR. MUSK forms a co-receptor for agrin with LRP4. Activation of MUSK by agrin and DOK7 results in the recruitment of several downstream kinases and phosphorylation of the AChR β-subunit, leading to the reorganization of the actin cytoskeleton and AChR clustering. The fundamental role of the MUSK-signaling pathway is supported by the fact that mice deficient in agrin, MUSK, rapsyn or Dok-7 lack postsynaptic differentiation and die at birth from respiratory failure.

CMS due to MUSK mutations manifests as respiratory insufficiency, neonatal ptosis, proximal limb muscle weakness, and weak bulbar, facial, or ocular muscles. A 30yo Chinese male with the LGMD-type of MUSK-related CMS developed mild atrophy of the leg muscles. LF-RNS was decremental. Pyridostigmin deteriorated the clinical manifestations. Another male infant manifested with congenital respiratory failure requiring mechanical ventilation, axial weakness with head drop, facial weakness, proximal limb weakness, and ophthalmoparesis. Salbutamol was effective but 3,4-DAP had only a mild effect, and AchE inhibitors worsened the phenotype. In a female with congenital hypotonia and respiratory distress requiring mechanical ventilation for 8 m, respiratory distress and nocturnal apnea with vocal cord paralysis recurred at age 8y. 3,4-DAP was effective. In two Turkish brothers MUSK mutations manifested as LGMD-type CMS. MUSK-related CMS may also manifest as congenital ptosis and later in life with fatigability. In another patient with MUSK-related CMS and congenital respiratory insufficiency, albuterol was moderately effective but AchE inhibitor, 3,4-DAP, and ephedrine were ineffective.

### Targeting RAPSN for the Prevention and or Treatment of CMS

Mutations in RAPSN cause defects within the AChR-clustering pathway. RAPSN encodes for rapsyn, a post-synaptic membrane protein that anchors the nicotinic AchR to the motor endplate and also binds to β-dystroglycan. Rapsyn is essential for clustering of the AchR at the post-synaptic membrane and required for the phosphorylation of CHRNB1. Mutant mice lacking rapsyn show absence of aggregation of AChRs and lack of accumulation of cytoskeletal proteins such as (3-dystroglycan, and utrophin.

RAPSN mutations are a common cause of post-synaptic CMS. Humans with mutations in the RAPSN gene are affected with a postsynaptic form of CMS characterized by impairment of the morphologic development of the postsynaptic region. The severity of symptoms in this form of CMS is variable. The most common of the RAPSN mutation is N88G, and patients are either homozygous or heterozygous for the N88K mutation (Ohno K, et al., (2002) Am JHum Genet 70(4):875-885).

Clinically, patients present with fluctuating ptosis, occasionally bulbar symptoms, neck muscle and mild proximal limb muscle weakness. Infections can precipitate exacerbation of clinical manifestations. In single patients prominent hyperlordosis can occur. Usually, the response to AchE inhibitor is favourable but can be improved by adding 3,4 DAP. Fluoxetine may worsen the decremental response in single patients. In some patients general anesthesia may exacerbate muscle weakness. The overall course is stable with intermittent worsenings.

### Targeting DOK7 for the Prevention and or Treatment of CMS

Mutations in DOK7 cause defects within the AChR-clustering pathway, and are responsible for about 10-20% of all cases of CMS. The DOK7 (downstream-of-kinase) gene encodes for the protein DOK7, which is involved in signaling downstream of receptor and non-receptor phosphotyrosine kinases. DOK7 is a cytoplasmic activator of muscle-specific receptor-tyrosine kinase (MuSK). Both DOK7 and MuSK are required for neuromuscular synaptogenesis. Mutations in DOK7 underlie a congenital myasthenic syndrome (CMS) associated with small and simplified neuromuscular synapses likely due to impaired DOK7/MuSK signaling. The overwhelming majority of patients with DOK7 CMS have at least one allele with a frameshift mutation that causes a truncation in the COOH-terminal region of DOK7 and affects MuSK activation.

Concerning the frequency of DOK7-related CMS, it was the second most frequent subtype in a Brasilian cohort. Clinical onset is characterised by gait disturbance due to muscle weakness after normal motor milestones. Proximal limb muscles are more strongly affected than distal limb muscles (LGMD-like pattern). Congenital DOK7-related CMS may manifest as stridor due to vocal cord paralysis, occasionally requiring intubation and artificial ventilation. Occasionally, patients present with ptosis but only rarely with ophthalmoparesis. Fatigability is often absent but prolonged periods of weakness may occur. Feeding difficulties may require nasogastral tube feeding or even PEG implantation. Muscle biopsy may show lipidosis and defective branching of terminal axons, which results in a unique terminal axon contacting en passant post-synaptic cups. AchE inhibitors are usually ineffective and may even worsen clinical manifestations. Ephedrine (initially 25 mg/d and increased to 75-100 mg/d) seems to be an effective alternative. Salbutamol may be effective in DOK7-related CMS as well. Single patients profit from albuterol, which can prevent progression of muscle weakness in LGMD-type DOK7-related CMS.

### Targeting SCN4A for the Prevention and or Treatment of CMS

Mutations in SCN4A cause defects within the AChR-clustering pathway. SCN4A encodes for a post-synaptic Nav1.4 voltage-gated sodium channel responsible for the initiation and propagation of the action potential in the muscle fibres that results in muscle contraction.

Several allelic disorders of skeletal muscle are caused by mutations of SCN4A. Missense mutations with gain-of-function changes (too much inward Na+ current) are found in hyperkalemic periodic paralysis (HyperPP), paramyotonia congenita, and several variants of sodium channel myotonia. Leaky channels resulting from mutations of arginine residues in the voltage sensor domain cause hypokalemic periodic paralysis (HypoPP) type 2. These traits are all dominantly inherited.

Loss-of-function (LOF) mutations of SCN4A are associated with recessively inherited phenotypes. A congenital myasthenic syndrome (CMS) has been associated with missense mutations of SCN4A that cause a LOF by markedly enhancing channel inactivation. More recently, congenital myopathy with neonatal hypotonia has been reported in patients with null mutations in SCN4A. A homozygous null is embryonic lethal, while compound heterozygous mutations (null allele plus an LOF allele) result in congenital myopathy with survival to adulthood. Remarkably, family members with a single SCN4A null allele are healthy.

Phenotypically, mutations in this gene manifest in infancy with global hypotonia, impaired sucking, dysphagia, delayed postural and motor development and later in life with episodic, fluctuating muscle weakness like in periodic paralysis, bilateral facial palsy, ptosis, and ophthalmoparesis. Episodes of periodic weakness could not be triggered by exercise, rest, potassium loading, or food, like in periodic paralysis. In older patients, SCN4A-related CMS may manifest exclusively as easy fatigability. In a 20yo normokalemic female, SCN4A-related CMS manifested as sudden attacks of respiratory and bulbar paralysis since birth, lasting 3-30 min and recurring one to three times per month, delayed motor development, easy fatigability, ptosis, ophthalmoparesis, and later as persisting facial, truncal, or limb weakness. Some patients present with dysmorphism, such as high-arched palate, adduction deformity of the knees or ankles, and increased lumbar lordosis. Some patients are mentally retarded with cerebral atrophy on MRI. RNS may be normal but higher stimulus frequency may trigger a decremental response. AchE inhibitors are only marginally effective. Acetazolamide together with potassium was ineffective.

### Targeting DUX4 for the Prevention and or Treatment of FSHD

Facioscapulohumeral muscular dystrophy (FSHD) is an autosomal dominant disorder primarily characterized by asymmetric, progressive muscle weakness beginning at the face, shoulders, and upper limbs, which spreads to the lower regions of the body with age. It is the third most common muscular dystrophy, with about 1:8,000-1:22,000 people affected worldwide. Age of onset is variable, ranging from birth to adulthood. Patients with the rare infantile form of FSHD, presenting symptoms before 5 y of age, follow a more severe and rapid course of the disease. At present, FSHD is incurable.

The majority of FSHD patients (˜95%, FSHD1) have a contraction of the D4Z4 repeat array in chromosome 4q35. Each D4Z4 repeat contains the first two exons of the double homeobox protein 4 (DUX4) gene, with its third (and final) exon located immediately downstream of the array. The D4Z4 array is normally hypermethylated in the course of development. Studies show that the contraction relaxes the chromatin and demethylates DNA in this region, resulting in aberrant DUX4 expression in skeletal muscle.

The aberrant expression of DUX4 in skeletal muscle is thought to cause FSHD. DUX4 encodes a transcription factor that activates pathways involved in muscle degeneration and apoptosis, events observed in patient muscles. DUX4 also inhibits myogenic differentiation and increases the sensitivity of muscle cells to oxidative stress. DUX4 is normally expressed during early embryonic development, and is then effectively silenced in all tissues except the testis and thymus. Its reactivation in skeletal muscle disrupts numerous signalling pathways that mostly converge on cell death. Thus, DUX4 serves as an attractive therapeutic target and inhibition of DUX4 expression represents be a potential therapy approach for FSHD.

### Targeting Adipose Tissue

In some embodiments, the present invention provides a double-stranded iRNA agent that targets DEGS1 for the treatment of a metabolic disorder.

In some embodiments, the present invention provides a double-stranded iRNA agent that targets leptin for the treatment of a metabolic disorder.

In some embodiments, the present invention provides a double-stranded iRNA agent that targets FLCN for the treatment of a metabolic disorder.

In some embodiments, the present invention provides a double-stranded iRNA agent that targets ZFP42 3 for the treatment of a metabolic disorder.

In some embodiments, the present invention provides a double-stranded iRNA agent that targets CDK6 the treatment of a metabolic disorder.

In some embodiments, the present invention provides a double-stranded iRNA agent that targets mTOR the treatment of a metabolic disorder.

In some embodiments, the present invention provides a double-stranded iRNA agent that targets RPTOR the treatment of a metabolic disorder.

In some embodiments, the present invention provides a double-stranded iRNA agent that targets FOXP1 the treatment of a metabolic disorder.

In some embodiments, the present invention provides a double-stranded iRNA agent that targets PDE3B the treatment of a metabolic disorder.

In some embodiments, the present invention provides a double-stranded iRNA agent that targets ACVR1C the treatment of a metabolic disorder.

### Targeting DEGS1 for the Prevention and or Treatment of a Metabolic Disorder

Obesity is a medical condition in which excess body fat has accumulated to the extent that it impairs health. The global epidemic of obesity is leading to unprecedented rates of diabetes and liver disease, and their late-stage complications, including cardiovascular and kidney disease.

The sphingolipid ceramide, which is a precursor to sphingomyelins and gangliosides that has both structural and signaling functions, is an important driver of the metabolic perturbations that underlie these diseases.

Dihydroceramide desaturase 1 (DEGS1), an enzyme that catalyzes the final step in the de novo synthesis of ceramide, is a particularly attractive therapeutic target. Indeed, studies in rodents reveal that inhibitors of ceramide synthesis prevent or reverse the pathogenic features of a metabolic disorder, including type 2 diabetes, nonalcoholic fatty liver disease, atherosclerosis, and cardiomyopathy. For example, Indeed, studies in rodents reveal that fenretinide, which directly targets and irreversibly inhibits DEGS1, prevented high fat diet-induced obesity and insulin resistance and hepatic steatosis without changes in energy expenditure or caloric intake (Preitner F, et al. Am J Physiol Endocrinol Metab. 2009; 279:E1420-E1429). Additionally, this DEGS1 inhibitor has been shown to sensitize obese post-menopausal women to insulin (Johansson H, et al. Cancer Res. 2008:68:9512-9518).

### Targeting Leptin for the Prevention and or Treatment of a Metabolic Disorder

Leptin, the product of the Lep gene, is a 167-residue peptide hormone. It is primarily secreted by adipose tissue. Functional inactivation of the Lep gene leads to undetectable levels of leptin in circulation.

Most common forms of obesity are associated with excessive circulating levels of leptin (coined “hyperleptinemia”), which results in a still ill-defined state of “leptin resistance”. The most accepted definition of leptin resistance is the inability of pharmacological doses of leptin to suppress food intake and body weight.

Hyperleptinemia is correlated with pro-inflammatory responses and with the chronic sub-inflammatory state observed in obesity. On one hand, leptin enhances the production of inflammatory cytokines, and on the other hand, cytokines such as IL-6 and TNF-α promote leptin production by the adipose tissue.

Increased leptin resistance associated with high levels of free fatty acid and inflammatory cytokines may contribute to the reduction in lipid oxidation in insulin-sensitive organs, leading to accumulation of lipids (lipotoxicity) and insulin resistance. In addition, leptin induces cholesterol uptake by macrophages, angiogenesis, platelet aggregation, stimulates the oxidative stress in endothelial cells and inhibits vasorelaxation, increasing the risk of atherosclerosis. Furthermore, in humans, leptin is an independent risk factor for coronary artery disease (Paz-Filho G, et al. Arq Bras Endocrinol Metab. 2012; 56/9:597-607).

### Targeting FLCN for the Prevention and or Treatment of a Metabolic Disorder

Non-alcoholic steatohepatitis (NASH) represents a major economic burden and is characterized by triglyceride accumulation, inflammation, and fibrosis. No pharmacological agents are currently approved to treat this condition.

Autophagy has been demonstrated to play a significant role in this condition, which serves to degrade intracellular lipid stores, reduce hepatocellular damage, and dampen inflammation. Autophagy is primarily regulated by the transcription factors TFEB and TFE3, which are negatively regulated by mTORC1. Given that FLCN is an mTORC1 activator via its GAP activity towards RagC/D, a liver specific Flcn knockout mouse model was generated to study its role in NASH progression. It was demonstrated that loss of FLCN results in reduced triglyceride accumulation, fibrosis, and inflammation in mice exposed to a NASH-inducing diet. (Paquette M, et al. BioRxiv. 2020).

It has also been demonstrated that FLCN regulates adipose tissue browning via mTOR and the transcription factor TFE3. Adipose-specific deletion of FLCN relieves mTOR-dependent cytoplasmic retention of TFE3, leading to direct induction of the PGC-1 transcriptional coactivators, drivers of mitochondrial biogenesis and the browning program.

### Targeting ZFP423 for the Prevention and or Treatment of a Metabolic Disorder

Obesity is a medical condition in which excess body fat has accumulated to the extent that it impairs health. The global epidemic of obesity is leading to unprecedented rates of diabetes and liver disease, and their late-stage complications, including cardiovascular and kidney disease.

Due to the global epidemic of obesity, there is an urgency to understand mechanisms regulating adipose development. Adipogenesis is initiated by the expression of ZFP423, which induces the expression of peroxisome proliferator-activated receptor γ (PPARGγ) and CCAAT-enhancer-binding proteins (C/EBPs), and genes specific for adipocytes.

Recently, ZFP423 was found to maintain white adipocyte identity by suppressing beige cell thermogenic gene progra). It has been shown in mice that adipocyte-specific inactivation of ZFP423 induced in adult mice leads to accumulation of beige-like adipocytes. The literature supports the notion that beige adipocytes can exert beneficial effects on glucose homeostasis and increase energy expenditure. Indeed, mice lacking adipocyte ZFP423 were resistant to diet-induced obesity. Furthermore, ZFP423 deficiency, combined with b3-adrenergic receptor activation, led to a reversal of weight gain and improved glucose tolerance when induced in obese animals (Shao M, et al. Cell Metabolism, 2016; 23:1167-1184).

### Targeting CDK6 for the Prevention and or Treatment of a Metabolic Disorder

Obesity has long been known to be the most important risk factor for the development of type II diabetes and other metabolic diseases. In rodents and humans, fat is deposited as energy storage in white adipose tissue (WAT), whereas fat is consumed to produce heat in the mitochondria-rich brown adipose tissues (BAT). As a thermogenic tissue, inducible-brown adipocytes (also called beige or brite cells) are found sporadically in WAT of adult animals with similar features as classical brown adipocytes. Importantly, the activation of beige cells is associated with a protection against obesity and metabolic diseases in rodent models and correlated with leanness in human

It has been shown that CDK6 regulates beige adipocyte formation and that mice lacking the CDK6 protein or its kinase domain (K43M) exhibit significant increases beige cell formation, enhanced energy expenditure, better glucose tolerance, and improved insulin sensitivity, and are more resistant to high-fat diet-induced obesity. Re-expression of CDK6 in Cdk6−/− mature or precursor cells, or ablation of RUNX1 in K43M mature or precursor cells, reverses these phenotypes (Hou X, et al. Nature Comm, 2018, 9: 1023). Additionally, overexpression of microRNA-107 (miR-107), which directly targets and downregulates CDK6, has been shown to reduce expression of CDK6 and its effectors and impairs adipocyte differentiation (Ahonen M A, et al. Molecular & Cellular Endocrin, 2019; 479:110-116). Thus, downregulation of CDK6 can potentially prevent and slow down progression of obesity.

### Targeting RPTOR for the Prevention and or Treatment of a Metabolic Disorder

Overnutrition causes hyperactivation of mTORC1-dependent negative feedback loops leading to the downregulation of insulin signaling and development of insulin resistance. Insulin signaling plays a crucial role in the control of systemic glucose homeostasis.

It has been shown that knockout of Rptor in different specific tissues of are protective against weight-gain and obesity. For example, a study in mice with Rptor conditionally deleted in osteoblast (Rptorob−/−) shows that, as compared to controls, chow-fed Rptorob−/− mice had substantially less fat mass and exhibited adipocyte hyperplasia. Remarkably, upon feeding with high-fat diet, mice with pre- and post-natal deletion of Rptor were protected from diet-induced obesity and exhibited improved glucose metabolism with lower fasting glucose and insulin levels, increased glucose tolerance and insulin sensitivity. This leanness and resistance to weight gain was not attributable to changes in food intake, physical activity or lipid absorption but instead was due to increased energy expenditure and greater whole-body substrate flexibility. RNA-seq revealed an increase in glycolysis and skeletal insulin signaling pathways, which correlated with the potentiation of insulin signaling and increased insulin-dependent glucose uptake in Rptor-knockout (Tangseefa P, et al. Bone Research, 2021; 9:10).

In another study, mice with an adipose-specific knockout of Rptor was generated, and these mice had substantially less adipose tissue, were protected against diet-induced obesity and hypercholesterolemia, and exhibited improved insulin sensitivity. Leanness was in spite of reduced physical activity and unaffected caloric intake, lipolysis, and absorption of lipids from the food (Polak P, et al. Cell Metabolism, 2008; 8(5):399-410). With similar profile, mice with Rptor knockout specifically in intestinal epithelial cells consistently gained less body weight on a high-fat diet compared to wildtype mice secondary to significantly reduced food intake. Importantly, the intestinal epithelial cell-specific Rptor knockout mice did not appear to be malnourished, demonstrated by their preservation of lean body mass, and also maintained a normal metabolic profile without significant changes in triglyceride or fasting glucose levels (Onufer E, et al. Biochem Biophys Res Comm, 2018; 505(4): 1174-1179).

Targeting mTOR for the Prevention and or Treatment of a Metabolic Disorder

The mTOR kinase is encoded by a single gene in mammals, but it exerts its main cellular functions by forming mTORC1 and mTORC2 through assembly with specific adaptor proteins. mTORC1 controls protein synthesis, cell growth and proliferation, and mTORC2 is a regulator of the actin cytoskeleton, and promotes cell survival and cell cycle progression. Cellular adenosine triphosphate (ATP) levels increase mTOR activity, and the mTOR kinase itself serves as a cellular ATP sensor. mTOR, thus, works as a critical checkpoint by which cells sense and decode changes in energy status

Overnutrition causes hyperactivation of mTORC1-dependent negative feedback loops leading to the downregulation of insulin signaling and development of insulin resistance. Insulin signaling plays a crucial role in the control of systemic glucose homeostasis.

In vitro experiments have demonstrated that mTORC1 is essential for the differentiation and maintenance of white adipocytes. Indeed, mTORC1 activation is necessary for insulin- or nutrient (amino acid)-induced adipogenesis and expression of SREBP1 and PPARγ, which are master transcriptional regulators of adipocyte differentiation and lipid homeostasis. Conversely, mTOR's inhibitor rapamycin impairs adipocyte differentiation by inhibiting PPARγ transactivation activity. In diet-induced obesity, overactivity of the mTORC1 signaling favors the expansion of the white adipose tissue mass, leading to adipocytes insulin resistance (Catania C, et al. IntJObes, 2011; 35:751-761).

### Targeting FOXP1 for the Prevention and or Treatment of a Metabolic Disorder

Obesity and its related complications such as type 2 diabetes mellitus, coronary heart disease and obstructive sleep apnea, have been considered significant health problems. Although dietary management, exercise, and pharmacological intervention have been proven to control weight, these approaches are largely inefficient for maintaining healthy long-term weight loss. Therefore, effective therapies for treating obesity and related metabolic disorders are needed.

Increasing brown adipose tissue (BAT) mass or/and activity in mice and humans has been demonstrated to help lose weight and improve whole-body metabolism.

In addition, adipose-specific deletion of FOXP1 led to an increase of brown adipose activity and browning program of white adipose tissues. The FOXP1-deficient mice showed an augmented energy expenditure and are protected from diet-induced obesity and insulin resistance. Consistently, overexpression of Foxp1 in adipocytes impaired adaptive thermogenesis and promotes diet-induced obesity.

In addition, deletion of the FOXP1 gene in osteoblasts led to augmentation of AdipoQ levels accompanied by fueled energy expenditure in adipose tissues. Adiponectin (AdipoQ) is a hormone abundantly secreted by adipose tissues, has multiple beneficial functions, including insulin sensitization as well as lipid and glucose metabolism. In contrast, overexpression of FOXP1 in bones impaired AdipoQ secretion and restrained energy consumption. Chromatin immunoprecipitation sequencing analysis revealed that AdipoQ expression, which increases as a function of bone age, is directly controlled by FOXP1 (Zhang W, et al. J Bone Miner Res, 2021).

### Targeting PDE3B for the Prevention and or Treatment of a Metabolic Disorder

The incidence of obesity in the developed world is increasing at an alarming rate. Concurrent with the increase in the incidence of obesity is an increase in the incidence of type 2 diabetes. Cyclic AMP (cAMP) and cGMP are key second messengers in all cells; for example, when it comes to processes of relevance for the regulation of energy metabolism, cAMP is a key mediator in the regulation of lipolysis, glycogenolysis, gluconeogenesis and pancreatic R cell insulin secretion.

PDE3B, one of several enzymes which hydrolyze cAMP and cGMP, is expressed in cells of importance for the regulation of energy homeostasis, including adipocytes, hepatocytes, hypothalamic cells and β cells. In adipocytes, PDE3B (phosphodiesterase 3B) is an important regulatory effector in signalling pathways controlled by insulin and cAMP-increasing hormones. Previous results from PDE3B-transgenic mice indicate that PDE3B, plays an important role in modulation of energy metabolism. In epididymal white adipose tissue of PDE3B KO mice on a SvJ129 background, cAMP/protein kinase A (PKA) and AMP-activated protein kinase (AMPK) signaling pathways are activated, resulting in “browning” phenotype, with a smaller increase in body weight under high-fat diet, smaller fat deposits, increased β-oxidation of fatty acids (FAO) and oxygen consumption (Chung Y W, et al. Scientific Report, 2017:7:40445).

In human, genome-wide analysis of array-based rare, non-synonymous variants in 184,246 individuals of UK Biobank and exome-sequence-based rare loss of function gene burden testing indicated that loss-of function of PDE3B is associated with a beneficial impact on waist-to-hip ration adjusted for BMI (WHRadjBMI).

### Targeting ACVR1C for the Prevention and or Treatment of a Metabolic Disorder

Body fat distribution strongly influences the development of type 2 diabetes. In a Mendelian randomization study of 296,291 individuals, it was previously found that a genetic predisposition to increased abdominal fat distribution was associated with elevated triglyceride levels, elevated blood pressure, and an increased risk of coronary artery disease, independent of overall adiposity. Furthermore, a genetic predisposition to increased abdominal fat distribution was strongly associated with the development of type 2 diabetes. For each 1 SD genetic increase in waist-to-hip ratio adjusted for BMI (WHRadjBMI) (a measure of body fat distribution), risk of type 2 diabetes increased by 77% (12). These findings were replicated in a separate Mendelian randomization study.

Recent studies of ALK7 indicate that ALK7's primary function in metabolic regulation is to limit catabolic activities and preserve energy. ALK7-knockout mice showed reduced diet-induced weight gain and fat accumulation when subjected to a high fat diet (Ibanez CA, FEBS J, 2021). In human, it has been demonstrated that variants predicted to lead to loss of function of the gene ACVR1C, which encodes the activin receptor-like kinase 7 (ALK7), influence body fat distribution and protect against type 2 diabetes (Emdin C A, et al. Diabetes, 2019:68(1):226-234). Genome-wide analysis of array-based rare, non-synonymous variants in 184,246 individuals of UK Biobank and exome-sequence-based rare loss of function gene burden testing indicated that loss-of function of ALK7 is associated with a beneficial impact on waist-to-hip ration adjusted for BMI (WHRadjBMI).

### XII. Kits

In certain aspects, the instant disclosure provides kits that include a suitable container containing a pharmaceutical formulation of a dsRNA agent of the invention. In certain embodiments, the individual components of the pharmaceutical formulation may be provided in one container. Alternatively, it may be desirable to provide the components of the pharmaceutical formulation separately in two or more containers, e.g., one container for a dsRNA agent and at least another for a pharmaceutically acceptable carrier, e.g., PBS. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

Unless otherwise defined, all technical and scientific terms used herein have the same meaning as commonly understood by one of ordinary skill in the art to which this invention belongs. Although methods and materials similar or equivalent to those described herein can be used in the practice or testing of the RNAi agents and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

An informal Sequence Listing is filed herewith and forms part of the specification as filed.

This invention is further illustrated by the following examples which should not be construed as limiting. The entire contents of all references, patents and published patent applications cited throughout this application, as well as the informal Sequence Listing and Figures, are hereby incorporated herein by reference.

### EXAMPLES

### Example 1: Synthesis of C₂₂-Nucleoside Phosphoramidites for the Synthesis of dsRNA Agent Conjugates

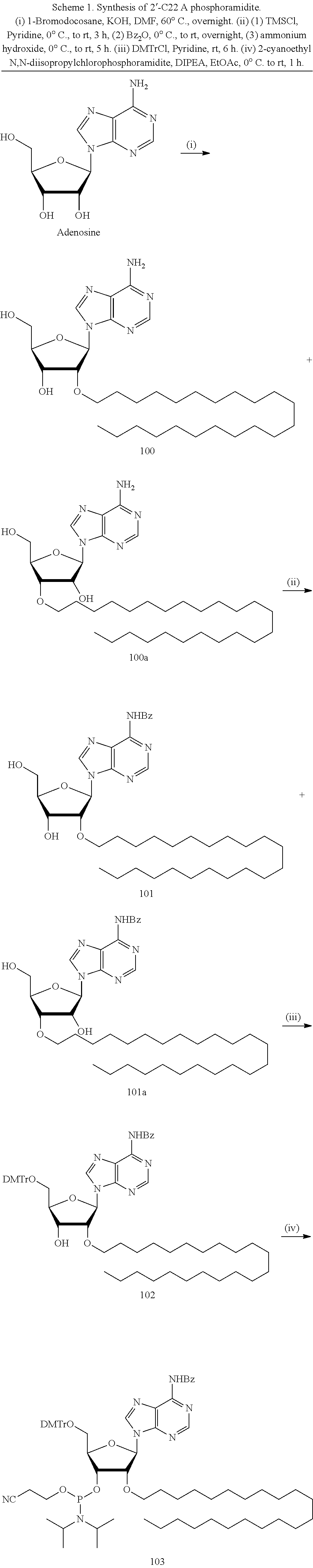

Compound 100: Adenosine (25 g, 93.6 mmol) and DMF (250 mL) were added into a 500 mL round-bottom flask, and then the suspension was warmed to 60° C. 1-Bromodocosane (54.7 g, 140 mmol) and KOH (10.5 g, 187 mmol) were added into the suspension and the reaction mixture was stirred at 60° C. overnight (16 h). The reaction was cooled to room temperature (a lot of insoluble matters were observed) and quenched by addition of NH₄Cl (10 g). The mixture, including the insoluble matter, was poured into a 2 μL separating funnel and diluted with CH₂C₁₂ and H₂O. (3 phases; organic phase, aqueous phase and emulsion phase, were observed.) The aqueous and emulsion phases were extracted with CH₂C₁₂ 3 times. TLC indicated that a major product spot was detected in the organic phase (5% MeOH in ethyl acetate, Rf=0.5). The collected organic phase was dried over anhydrous sodium sulfate and concentrated under vacuum. The crude compound was purified by column chromatography on silica gel (0-10% MeOH in CH₂Cl₂ for 60 min) to obtain a mixture of 2′-C₂₂ product (compound 100) and 3′-C₂₂ product (100a) as a white solid (28.2 g, 52%; 2′-C22/3′-C₂₂=ca. 9:1). ¹H NMR (600 MHz, DMSO-d₆) δ 8.38-8.33 (m, 1H), 8.13 (s, 1H), 7.35 (brs, 2H), 5.98 (d, J=6.3 Hz, 0.9H), 5.78 (d, J=6.2 Hz, 0.1H), 5.47-5.40 (m, 1H), 5.19-5.16 (m, 1H), 4.73 (q, J=5.8 Hz, 0.1H), 4.47 (dd, J=4.8, 6.4 Hz, 0.9H), 4.30-4.28 (m, 0.9H), 4.04-4.03 (m, 0.1H), 3.99-3.97 (m, 0.9H), 3.94-3.92 (m, 0.1H), 3.69-3.66 (m, 1H), 3.58-3.53 (m, 2H), 3.37-3.30 (m, 1H), 1.58-1.53 (m, 0.2H), 1.39-1.36 (m, 1.8H), 1.29-1.08 (m, 38H), 0.85 (t, J=6.6 Hz, 3H). LCMS (ESI) calculated for C₃₂H₅₈N₅O₄ [M+H]⁺ m/z=576.45, found 576.4.

Compound 101: To a suspension of compound 100 and 3′-C22 100a (mixture, 28 g, 48.6 mmol) in pyridine (40 mL) was added dropwise TMSCl (2.98 mL, 23.4 mmol) at 0° C. and the mixture was warmed to room temperature and stirred for 3 h. TLC indicated that compound 100 and 100a was consumed, and a new major spot was detected (50% ethyl acetate in hexane, Rf=0.7). The reaction mixture was cooled to 0° C. and benzoic anhydride (2.12 g, 9.38 mmol) was added. The resulting solution was wormed to room temperature and stirred overnight (14 h). TLC indicated that the protected intermediate with TMS was consumed, and a new major spot was detected (50% ethyl acetate in hexane, Rf=0.8). The reaction was cooled to 0° C. and quenched with H₂O. The resulting solution was warmed to room temperature and stirred for 5 h. The mixture was cooled to 0° C. and 28% ammonium hydroxide solution (40 mL) was added. The resulting mixture was warmed to room temperature and stirred for 5 h. TLC indicated that the fully protected intermediate with TMS and Bz groups was consumed, and a new major spot was detected (100% ethyl acetate, Rf=0.5). The reaction was diluted with ethyl acetate and the organic layer was washed with water, brine and dried (Na₂SO₄) and concentrated under vacuum. The crude residue was purified by column chromatography on silica gel (0-5% MeOH in CH₂Cl₂ for 30 min) to obtain a mixture of 2′-C₂₂ product (compound 101) and 3′-C₂₂ product (101a) as a white solid (24.9 g, 75%; 2′-C22/3′-C₂₂=ca. 9:1). ¹H NMR (600 MHz, DMSO-d₆) δ 11.22 (s, 1H), 8.75 (s, 1.8H), 8.73 (s, 0.2H), 8.06-8.04 (m, 2H), 7.66-7.63 (m, 1H), 7.57-7.54 (m, 2H), 6.14 (d, J=5.9 Hz, 0.9H), 6.04 (d, J=5.8 Hz, 0.1H), 5.53 (d, J=6.2 Hz, 0.1H), 5.23 (d, J=5.3 Hz, 0.9H), 5.17 (brs, 1H), 4.79 (q, J=5.5 Hz, 0.1H), 4.52 (dd, J=5.0, 6.1 Hz, 0.9H), 4.34 (q, J=4.2 Hz, 0.9H), 4.06 (q, J=3.9 Hz, 0.1H), 4.04-3.98 (m, 1H), 3.71-3.69 (m, 1H), 3.62-3.59 (m, 2H), 3.52-3.48 (m, 0.1H), 3.42-3.39 (m, 0.9H), 1.58-1.53 (m, 0.2H), 1.44-1.40 (m, 1.8H), 1.26-1.11 (m, 38H), 0.84 (t, J=6.8 Hz, 3H). LCMS (ESI) calculated for C₃₉H₆₂N5O₅ [M+H]⁺m/z=680.48, found 680.6.

Compound 102: To a solution of compound 101 and 3′-C₂₂ product 101a (mixture, 25 g, 36.8 mmol) in pyridine (300 mL) was added 4,4′-dimethoxytriphenyl chloride (12.5 g, 36.8 mmol) and the mixture was stirred at room temperature for 6 h. TLC indicated that compound 101 was consumed, and a new major spot was detected (90% ethyl acetate in hexane, Rf=0.8). The reaction was quenched with saturated NaHCO₃ (aq.) and diluted with ethyl acetate. The organic layer was washed with water, brine and dried (Na₂SO₄) and concentrated under vacuum. The crude residue was purified by column chromatography on silica gel (0-50% ethyl acetate in hexane for 10 min and then kept 50% ethyl acetate in hexane for 10 min) to obtain compound 102 as a light-yellow form (28.5 g, 79%). ¹H NMR (600 MHz, DMSO-d₆) δ 11.23 (brs, 1H), 8.66 (s, 1H), 8.60 (s, 1H), 8.06-8.04 (m, 2H), 7.66-7.63 (m, 1H), 7.56-7.54 (m, 2H), 7.38-7.36 (m, 2H), 7.27-7.18 (m, 7H), 6.85-6.82 (m, 4H), 6.15 (d, J=5.3 Hz, 1H), 5.26 (d, J=5.8 Hz, 1H), 4.66 (t, J=5.1 Hz, 1H), 4.39 (q, J=5.1 Hz, 1H), 4.12 (q, J=4.5 Hz, 1H), 3.72 (s, 6H), 3.61 (dt, J=6.5, 9.7 Hz, 1H), 3.45 (dt, J=6.5, 9.7 Hz, 1H), 3.30-3.24 (m, 2H), 1.47-1.42 (m, 2H), 1.27-1.12 (m, 38H), 0.84 (t, J=6.8 Hz, 3H). LCMS (ESI) calculated for C₆₀HoN₅O₇[M+H]⁺ m/z=982.61, found 982.6.

Compound 103: To a solution of compound 102 (5 g, 5.09 mmol) in ethyl acetate (30 mL) was added dropwise 2-cyanoethyl N,N-diisopropylchlorophosphoramidite (1.48 mL, 6.62 mmol) at 0° C. and the mixture was stirred at room temperature for 1 h. TLC indicated that compound 102 was consumed, and a new major spot was detected (40% ethyl acetate in hexane, Rf=0.4). The reaction mixture was quenched with saturated NaHCO₃ (aq.) and then the organic layer was washed with water, brine, dried (Na₂SO₄) and concentrated under vacuum. The crude residue was purified by column chromatography on silica gel (0-40% ethyl acetate in hexane for 15 min and then 40-50% ethyl acetate in hexane for 20 min) to obtain compound 103 as a white form (4.58 g, 76%). ¹H NMR (600 MHz, CD₃CN) δ 9.36 (brs, 1H), 8.62-8.60 (m, 1H), 8.31-8.29 (m, 1H), 8.02-8.00 (m, 2H), 7.67-7.64 (m, 1H), 7.57-7.55 (m, 2H), 7.47-7.43 (m, 2H), 7.35-7.21 (m, 7H), 6.86-6.82 (m, 4H), 6.14-6.13 (m, 1H), 4.84-4.81 (m, 1H), 4.74-4.69 (m, 1H), 4.37-4.31 (m, 1H), 3.95-3.63 (m, 11H), 3.56-3.52 (m, 1H), 3.50-3.45 (m, 1H), 3.39-3.33 (m, 1H), 2.74-2.65 (m, 1H), 2.52 (t, J=6.1 Hz, 1H), 1.53-1.48 (m, 2H), 1.34-1.19 (m, 47H), 1.12 (d, J=6.8 Hz, 3H), 0.90 (t, J=6.8 Hz, 3H). ³¹P NMR (243 MHz, CD₃CN) δ 149.89, 149.85, 149.81, 149.77, 149.74, 149.70, 149.48, 149.44, 149.40, 149.36, 149.32, 149.28. LCMS (ESI) calculated for C₆₉H₉₇N70₈P [M+H]⁺ m/z=1182.71, found 1182.6.

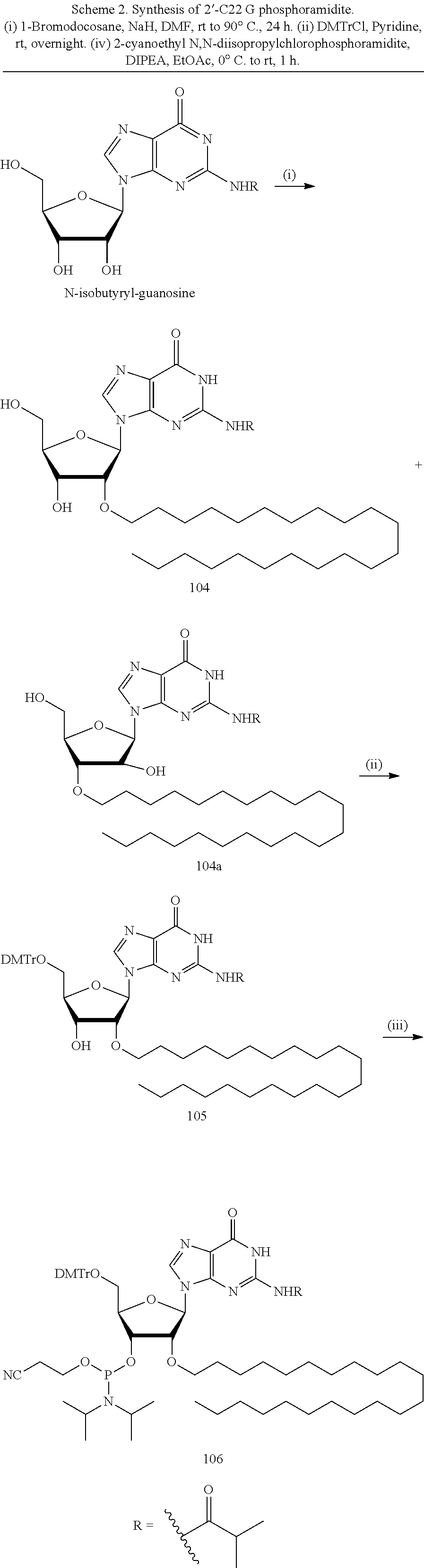

Compound 104: N-Isobutyrylguanosine (5 g, 14.2 mmol) and DMF (50 mL) were added into a 250 mL round-bottom flask, and then the solution was cooled to 0° C. NaH (60% in mineral oil; 1.42 g, 35.4 mmol) was added portion-wise into the solution and the suspension was stirred at 0° C. for 30 min. To the mixture was added 1-bromodocosane (8.27 g, 21.2 mmol). The suspension was wormed up to 90° C. and stirred for 24 h. TLC indicated that N-isobutyrylguanosine remained, and a new major spot was detected (100% ethyl acetate, Rf=0.4). The reaction mixture was cooled to 0° C. and quenched by addition of NH₄C₁ (a lot of insoluble matters were observed). The mixture, including the insoluble matter, was poured into a 1 μL separating funnel and diluted with CH₂C₁₂ and H₂O. (3 phases; organic phase, aqueous phase and emulsion phase, were observed.) The aqueous and emulsion phases were extracted with CH₂C₁₂ 3 times. The collected organic phase was dried over anhydrous sodium sulfate (Na₂SO₄) and concentrated under vacuum. The crude compound was purified by column chromatography on silica gel (0-10% MeOH in CH₂Cl₂ for 30 min) to obtain a mixture of 2′-C₂₂ (compound 104)/3′-C₂₂ products(104a), contained some other impurities, as a white solid (2.10 g, 22%). LCMS (ESI) calculated for C₃₆H₆₄N5O₆ [M+H]⁺ m/z=662.49, found 662.6. The obtained compound 104 was used for the next reaction without any further purifications.

Compound 105: To a solution of compound 104 (2.1 g, 3.17 mmol) in pyridine (30 mL) was added 4,4′-dimethoxytriphenyl chloride (1.07 g, 3.17 mmol) and the mixture was stirred at room temperature for overnight (14 h). TLC indicated that compound 104 was consumed, and a new major spot was detected (80% ethyl acetate in hexane, Rf=0.8). The reaction was quenched with saturated NaHCO₃ (aq.) and diluted with ethyl acetate. The organic layer was washed with water, brine and dried (Na₂SO₄) and concentrated under vacuum. The crude residue was purified by column chromatography on silica gel (0-45% ethyl acetate in hexane for 10 min and then kept 45% ethyl acetate in hexane for 30 min) to obtain compound 105 as a white form (2.13 g, 70%). ¹H NMR (600 MHz, DMSO-d₆) δ 12.10 (brs, 1H), 11.64 (brs, 1H), 8.12 (s, 1H), 7.36-7.34 (m, 2H), 7.27-7.19 (m, 7H), 6.85-6.81 (m, 4H), 5.93 (d, J=5.8 Hz, 1H), 5.18 (d, J=5.4 Hz, 1H), 4.40 (t, J=5.4 Hz, 1H), 4.26 (q, J=4.8 Hz, 1H), 4.05 (ddd, J=3.5, 3.5, 6.0 Hz, 1H), 3.73 (s, 6H), 3.59 (dt, J=6.4, 9.7 Hz, 1H), 3.45 (dt, J=6.4, 9.7 Hz, 1H), 3.30 (d, J=6.0, 10.5 Hz, 1H), 3.16 (d, J=3.5, 10.5 Hz, 1H), 2.76 (sept, J=6.8 Hz, 1H), 1.44-1.40 (m, 2H), 1.26-1.11 (m, 44H), 0.84 (t, J=6.8 Hz, 3H). LCMS (ESI) calculated for C₅₇H₈₂N₅O₈[M+H]⁺ m/z=964.62, found 964.6.

Compound 106: To a solution of compound 105 (2 g, 2.07 mmol) and DIPEA (0.470 mL, 2.70 mmol) in ethyl acetate (20 mL) was added dropwise 2-cyanoethyl N,N-diisopropylchlorophosphoramidite (0.601 mL, 2.70 mmol) at 0° C. and the mixture was stirred at room temperature for 1 h. TLC indicated that compound 105 was consumed, and a new major spot was detected (50% ethyl acetate in hexane, Rf=0.6). The reaction mixture was washed with saturated NaHCO₃ (aq.), water, brine, dried (Na₂SO₄) and concentrated under vacuum. The crude residue was purified by column chromatography on silica gel (0-50% ethyl acetate in hexane for 30 min and then kept 50% ethyl acetate in hexane for 10 min) to obtain compound 106 as a white form (1.76 g, 73%). ¹H NMR (600 MHz, CD₃CN) δ 11.99 (brs, 1H), 9.16 (brs, 1H), 7.89-7.87 (m, 1H), 7.49-7.23 (m, 9H), 6.87-6.83 (m, 4H), 5.92 (t, J=6.1 Hz, 1H), 4.68-4.63 (m, 1H), 4.53-4.47 (m, 1H), 4.34-4.26 (m, 1H), 3.92-3.60 (m, 11H), 3.54-3.49 (m, 1H), 3.40 (d, J=4.0 Hz, 1H), 3.35 (d, J=4.0 Hz, 1H), 2.73-2.65 (m, 1H), 2.57-2.47 (m, 2H), 1.53-1.47 (m, 2H), 1.34-1.18 (m, 47H), 1.15-1.06 (m, 9H), 0.90 (t, J=6.7 Hz, 3H). ³¹P NMR (243 MHz, CD₃CN) δ 149.80, 149.76, 149.72, 149.68, 149.64, 149.60, 149.56, 149.51, 149.48, 149.44. LCMS (ESI) calculated for C₆₆H₉₉N70₉P [M+H]⁺ m/z=1164.72, found 1164.8.

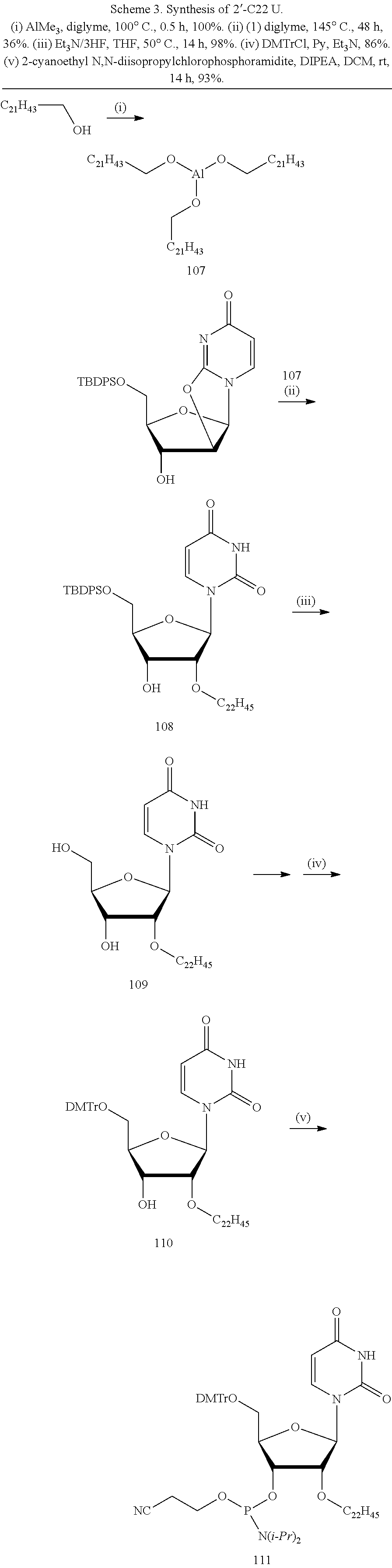

Compound 107: 2M solution of AlMe₃ (50 mL, 0.10 mol) was added slowly for ca. 15 min to a stirred suspension of 1-docosanol (108 g, 0.33 mol) in anhyd. diglyme (90 mL) under Ar atmosphere in a 2-neck 1 μL flask fitted with a magnetic stirring bar, and an outlet with a gas bubbler over a reflux condenser. After completion of the addition, the mixture was heated to 100° C. until evolution of gas through the bubbler was complete (30 min). The mixture was cooled to 65° C. and diluted with anhyd. AcOEt (150 mL) and anhyd. ACN (150 mL). The mixture was cooled down to rt in the bath overnight, a white residue formed was filtered through a 600 mL glass filtering funnel under the cushion of Ar, and washed with a 1:1 mixture of anhyd. AcOEt and anhyd. ACN (400 mL×2) under the cushion of Ar. The residue was dried on the funnel in reverse flow of nitrogen, transferred to a flask and dried in high vacuum for 24 h to afford 107.4 g of the alkoxide 107 of ca. 93% purity containing ca. 7% of 1-docosanol that was used in the next step without of further purification. The product was stored under Ar atmosphere.

Compound 108: A mixture of 5′-TBDPS-protected anhydro-uridine (18.6 g, 40 mmol), aluminum alkoxide 107 (˜93%, 47.6 g, 44 mmol) and anhyd. diglyme (60 mL) was heated to 145° C. bath temperature in a flask fitted with a magnetic stirring bar and a reflux condenser under slight positive pressure of Ar using a balloon for 48 h. The mixture was cooled down to 70° C. in the bath, diluted with AcOEt (200 mL), further cooled down 30° C. and quenched by addition of 10% H₃PO₄ (200 mL). A suspension thus formed was stirred at rt overnight, filtered through a 600 mL glass filtering funnel, and the solids were washed thoroughly with water (ca. 50 mL) and AcOEt (ca. 300 mL) mixture. Thoroughly compressed solid residue was dried in warm air to afford 25.4 g (55%) of recovered 1-docosanol. The filtrate was transferred to a separatory funnel, the organic layer was separated, washed with 1% NaCl (500 mL×2), saturated NaCl (200 mL) and dried over anhyd. Na₂SO₄. The solvent was removed in vacuo, the residue was co-evaporated with additional portion of AcOEt (300 mL) to afford 61.8 g of crude residue. The latter was dissolved in 190 mL of AcOEt-hexanes 1:4 mixture and liquid-loaded on a standard 330 g column of silica gel. The column was eluted with isocratic 20% AcOEt in hexanes followed by gradient of 20 to 40% of AcOEt in hexanes, the fractions containing product were pulled, evaporated in vacuum, co-evaporated twice with ACN-diethyl ether mixture, and dried in high vacuum to afford 11.4 g (36%) of pure product 108. ¹H NMR (600 MHz, Acetone-d6) δ 10.08 (s, 1H), 7.88 (d, J=7.8 Hz, 1H), 7.80-7.76 (m, 2H), 7.76-7.73 (m, 2H), 7.53-7.44 (m, 6H); 6.00 (d, J=3.0 Hz, 1H), 5.27 (d, J=8.4 Hz, 1H), 4.46 (q, J=5.4 Hz, 1H), 4.12 (dd, J=12.0, 2.4 Hz, 1H), 4.08-4.04 (m, 2H), 3.98 (dd, J=11.4, 2.4 Hz, 1H), 3.95 (d, J=7.2 Hz, 1H), 3.78 (dt, J=9.6, 6.6 Hz, 1H), 3.69 (dt, J=9.6, 6.6 Hz, 1H), 1.65-1.59 (m, 2H), 1.43-1.36 (m, 2H), 1.35-1.25 (m, 36H), 1.13 (s, 9H), 0.89 (t, J=6.6 Hz, 3H). MS (ESI+ APCI), calculated for C₄₇H₇₄N₂O₆Si [M+H]⁺ exact mass m/z=791.54, found 791.7.

Compound 109: A mixture of TBDPS-protected nucleoside 108 (2.25 g, 2.8 mmol), anhyd. THF (10 mL), and triethylamine trihydrofluoride (2 mL, 12 mmol) was heated at 50° C. under Ar atmosphere for 24 h. Heptane (40 mL) followed by water (40 mL) were added, the heating bath was removed, the mixture was stirred overnight at rt, filtered, and washed thoroughly by water-heptane mixture. The solid was dried in the flow of nitrogen for 2 h followed by warm air overnight to afford 1.52 g (98%) of 109 as a white solid. ¹H NMR (600 MHz, Acetone-d6) δ 10.01 (s, 1H), 7.88 (d, J=8.4 Hz, 1H), 5.97 (d, J=4.2 Hz, 1H), 5.60 (d, J=8.4 Hz, 1H), 4.37 (s, 1H), 4.35-4.29 (m, 1H), 4.06 (t, J=4.8 Hz, 1H), 4.00 (dt, J=5.4, 2.4 Hz, 1H), 3.93-3.87 (m, 1H), 3.84 (d, J=6.6 Hz, 1H), 3.83-3.78 (m, 1H), 3.74-3.63 (m, 2H), 1.64-1.56 (m, 2H), 1.43-1.23 (m, 38H), 0.89 (t, J=6.6 Hz, 3H). MS (ESI+ APCI), calculated for C₃₁H₅₆N₂O₆ [M+H]⁺ exact mass m/z=553.42, found 553.5.

Compound 110: Triethylamine (0.77 mL, 5.5 mmol) was added to a solution of nucleoside 109 (1.49 g, 2.7 mmol) and DMTrCl (1.86 g, 5.5 mmol) in anhyd. pyridine (10 mL) under Ar atmosphere. The mixture was stirred at rt overnight, quenched by addition of MeOH (0.2 mL), and diluted with ACN (25 mL). The solvents were evaporated in vacuo at 25° C., the residue was co-evaporated twice with ACN at 25° C. and partitioned between AcOEt and 5% NaCl. The organic phase was separated, washed with sat. NaCl, and dried over anhyd. Na₂SO₄. The solvent was removed in vacuo to afford 3.50 g of crude product. The latter was purified by chromatography over a standard 40 g column of silicagel with isocratic 30% of AcOEt in hexanes followed by gradient of 30 to 50% of AcOEt in hexanes. Fractions contained product were pulled evaporated in vacuum, co-evaporated twice with ACN-diethyl ether mixture, and dried in high vacuum to afford 1.98 g (86%) of product 110 as a yellowish foam. ¹H NMR (600 MHz, Acetone-d6) δ 10.08 (s, 1H), 7.93 (d, J=7.8 Hz, 1H), 7.52-7.48 (m, 2H), 7.40-7.33 (m, 6H), 7.29-7.26 (m, 1H), 6.93 (d split, J=9.0 Hz, 4H), 5.96 (d, J=2.4 Hz, 1H), 5.27 (d, J=7.8 Hz, 1H), 4.52-4.48 (m, 1H), 4.12-4.07 (m, 2H), 3.94 (d, J=7.8 Hz, 1H), 3.85-3.77 (m, 1H), 3.81 (s, 6H), 3.73-3.68 (m, 1H), 3.52 (dd, J=10.8, 3.6 Hz, 1H), 3.46 (dd, J=10.8, 3.0 Hz, 1H), 1.67-1.59 (m, 2H), 1.43-1.56 (m, 2H), 1.36-1.23 (m, 36H), 0.89 (t, J=6.6 Hz, 3H). MS (ESI+ APCI), calculated for C₅₂H₇₄N₂O₈[M+H]⁺ exact mass m/z=854.54.

Compound 111: DIPEA (0.46 mL, 2.6 mmol) followed by 2-cyanoethyl N,N-diisopropylchlorophosphoramidite (0.57 mL, 2.6 mmol) were added to a solution of compound 110 (1.71 g, 2 mmol) in anhyd. DCM (10 mL) under Ar. The mixture was stirred at rt for 14 h, cooled to 0° C., quenched by addition of sat. NaHCO₃ and extracted with AcOEt (25 mL). Organic phase was separated, washed with sat. NaCl, and dried over anhyd. sodium sulfate. Crude material (2.20 g) was purified over a standard 40 g flash column of silica gel that was eluted with isocratic 50% of AcOEt containing 0.3% of TEA in hexanes to afford 1.96 g (93%) of 111 as a white foam. ¹H NMR (500 MHz, CD3CN) δ 8.93 (s, 1H), 7.82-7.70 (m, 1H), 7.47-7.41 (m, 2H), 7.36-7.23 (m, 8H), 6.91-6.85 (m, 4H), 5.87-5.82 (m, 1H), 5.24-5.19 (m, 1H), 4.50-4.38 (m, 1H), 4.19-4.11 (m, 1H), 4.06-3.99 (m, 1H), 3.77 (s, 3H), 3.77 (s, 3H), 3.63-3.59 (m, 2H), 3.45-3.41 (m, 1H), 2.72-2.59 (m, 1H), 2.52 (t, J=6.0 Hz, 1H), 1.60-1.50 (m, 2H), 1.42-1.07 (m, 51H), 1.06 (d, J=6.8 Hz, 3H), 0.90-0.84 (m, 3H). C13 NMR (151 MHz, CD3CN) δ 163.47, 163.43, 159.33, 159.31, 159.30, 150.84, 145.32, 145.25, 140.54, 140.51, 135.99, 135.95, 135.85, 135.77, 130.76, 130.71, 130.69, 128.63, 128.60, 128.51, 127.57, 119.12, 119.00, 113.68, 113.67, 102.11, 102.02, 88.36, 88.23, 87.22, 87.18, 82.95, 82.93, 82.80, 82.76, 81.98, 81.25, 81.22, 71.24, 71.00, 70.76, 70.66, 70.59, 70.51, 62.45, 62.00, 59.15, 59.02, 58.78, 58.64, 55.48, 55.46, 43.58, 43.54, 43.50, 43.45, 32.21, 30.01, 29.99, 29.96, 29.92, 29.85, 29.73, 29.69, 29.65, 26.32, 26.30, 24.67, 24.62, 24.57, 24.52, 24.48, 24.46, 24.41, 22.96, 20.65, 20.60, 13.98. P31 NMR (202 MHz, CD3CN) δ 149.47, 149.08.

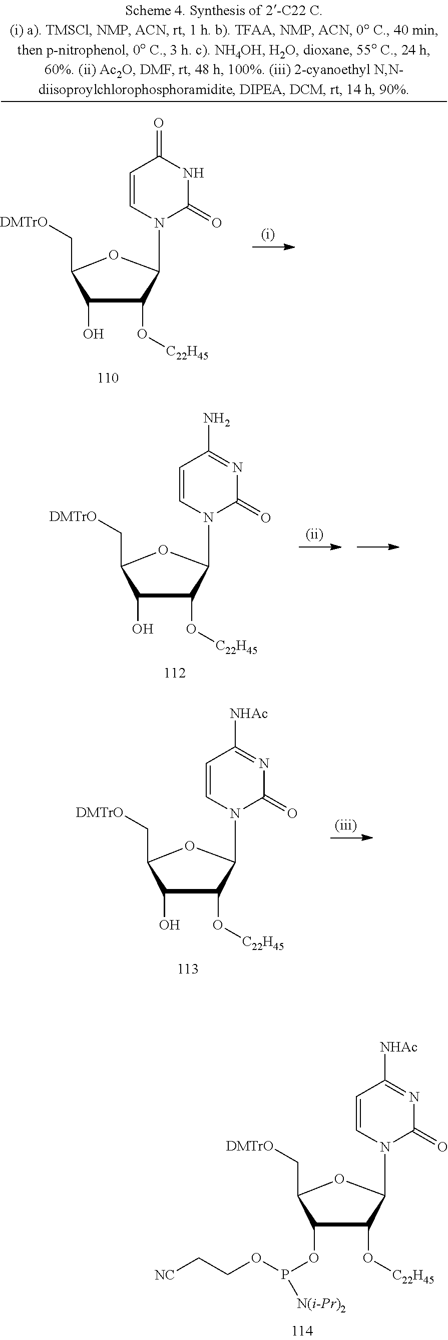

Compound 112: TMSCl (0.4 mL, 3.2 mmol) was added to a solution of 110 (1.20 g, 1.4 mmol) and NMP (1.2 mL, 11.8 mmol) in anhyd. MeCN (7 mL) under Ar atmosphere. The mixture was stirred at rt for 1 h, cooled to 0° C., and TFAA (0.5 mL, 3.6 mmol) was added slowly dropwise via syringe. The mixture was stirred at 0° C. for 40 min, and p-nitrophenol (0.56 g, 4 mmol) was added. The mixture was stirred at 0° C. for 3 h and quenched by addition of sat. sodium bicarbonate (15 mL). The cooling bath was removed, ethyl acetate (30 mL) was added, followed by minimal amount of water to dissolve inorganic precipitates. The organic phase was separated, washed with sat. NaCl, dried over anhyd. sodium sulfate and evaporated in vacuum to afford 1.94 g of oily residue. The latter was dissolved in dioxane (15 mL), the solution was transferred to a pressure bottle, saturated ammonium hydroxide solution (2.2 mL) was added, and the bottle was heated at 55° C. with stirring for 24 h. The mixture was cooled to rt, the solvent was evaporated in vacuum and the residue (2.40 g) was chromatographed over a column of silica gel with gradient of methanol in ethyl acetate (0 to 6%). The fraction containing product were pulled, evaporated in vacuum, and the residue was treated with 5 mL of ACN that triggered extensive crystallization. The mixture was kept at 0° C. for 4 h, filtered, the crystalline residue was washed with ACN, and air-dried to afford 0.73 g (60%) of C-22 cytidine 112 as a white solid. ¹H NMR (400 MHz, DMSO-d6) δ 7.77 (d, J=7.2 Hz, 1H), 7.42-7.35 (m, 2H), 7.31 (t, J=7.2 Hz, 2H), 7.28-7.22 (m, 5H), 7.22-7.12 (m, 2H), 6.89 (d, J=8.8 Hz, 4H), 5.80 (d, J=2.8 Hz, 1H), 5.48 (d, J=7.6 Hz, 1H), 4.98 (d, J=6.8 Hz, 1H), 4.19-4.11 (m, 1H), 3.99-3.90 (m, 1H), 3.77-3.69 (m, 1H), 3.73 (s, 6H), 3.68-3.52 (m, 2H), 3.30-3.22 (m, 2H), 1.55-1.45 (m, 2H), 1.34-1.14 (m, 38H), 0.83 (t, J=6.8 Hz, 3H). MS (ESI+ APCI), calculated for C₅₂H₇₅N₃₀₇ [M+H]⁺ exact mass m/z=854.57.

Compound 113: C-22-Cytidine 112 (0.68 g, 0.8 mmol) was dissolved in anhyd. DMF (4 mL) under Ar atmosphere, and acetic anhydride (0.09 mL, 0.9 mmol) was added. The mixture was stirred at rt for 48 h, cooled to 0° C., quenched by addition of 5% NaCl (10 mL), and diluted with ethyl acetate (10 mL). The organic phase was separated, washed with 5% NaCl (2×20 mL), sat. sodium bicarbonate, sat. NaCl, and dried over anhyd. sodium sulfate. The solvent was removed in vacuum and the residue was co-evaporated twice with ACN-diethyl ether mixture to afford 0.72 g (100%) of C-22 N(Ac)-cytidine 113 as a white solid. ¹H NMR (400 MHz, DMSO-d6) δ 10.90 (s, 1H), 8.28 (d, J=7.6 Hz, 1H), 7.41-7.35 (m, 2H), 7.31 (t, J=7.2 Hz, 2H), 7.28-7.21 (m, 5H), 7.00 (d, J=7.2 Hz, 1H), 6.92-6.86 (m, 4H), 5.79 (d, J=1.2 Hz, 1H), 5.05 (d, J=7.2 Hz, 1H), 4.25-4.18 (m, 1H), 4.05-3.99 (m, 1H), 3.78 (dd, J=4.8, 1.2 Hz, 1H), 3.77-3.69 (m, 1H), 3.74 (s, 6H), 3.65-3.57 (m, 1H), 3.37-3.27 (m, 2H), 2.08 (s, 3H), 1.57-1.48 (m, 2H), 1.35-1.14 (m, 38H), 0.83 (t, J=6.8 Hz, 3H). MS (ESI+ APCI), calculated for C₅₄H₇₇N₃O₅ [M+H]⁺ exact mass m/z=896.58.

Compound 114: DIPEA (0.16 mL, 0.9 mmol) followed by 2-cyanoethyl N,N-diisopropylchlorophosphoramidite (0.20 mL, 0.9 mmol) were added to a solution of compound 113 (0.63 g, 0.7 mmol) in anhyd. DCM (5 mL) under Ar. The mixture was stirred at rt for 14 h, cooled to 0° C., quenched by addition of sat. NaHCO₃ and extracted with AcOEt (15 mL). Organic phase was separated, washed with sat. NaCl, and dried over anhyd. sodium sulfate. Crude material (0.82 g) was purified over a standard 24 g flash column of silica gel that was eluted with isocratic 70% of AcOEt containing 0.3% of TEA in hexanes followed by gradient 70 to 100% of AcOEt containing 0.3% of TEA in hexanes to afford 0.69 g (90%) of 114 as a white foam. ¹H NMR (600 MHz, DMSO) δ 10.94 (s, 1H), 8.46-8.34 (m, 1H), 7.46-7.36 (m, 2H), 7.36-7.20 (m, 7H), 6.98 (t, J=8.4 Hz, 1H), 6.89 (t, J=8.6 Hz, 4H), 5.85 (d, J=13.8 Hz, 1H), 4.55-4.34 (m, 1H), 4.20-4.11 (m, 1H), 4.06-3.83 (m, 2H), 3.75 (s, 3H), 3.74 (s, 3H), 3.68-3.64 (m, 1H), 3.50-3.44 (m, 2H), 2.77-2.69 (m, 1H), 2.66-2.59 (m, 1H), 2.10 (s, 3H), 1.56-1.49 (m, 2H), 1.26-1.16 (m, 51H), 0.96 (d, J=6.7 Hz, 3H), 0.85-0.82 (m, 3H). P31 NMR (243 MHz, DMSO) δ 149.23, 147.94.

### Example 2. dsRNA Synthesis

### Source of Reagents

Where the source of a reagent is not specifically given herein, such reagent can be obtained from any supplier of reagents for molecular biology at a quality/purity standard for application in molecular biology.

siRNA Design

siRNAs targeting the human adrenoceptor beta 1 (ADRB1) gene (human: GenBank NM_000684.3, NCBI GeneID: 153) were designed using custom R and Python scripts. The human ADRB1 REFSEQ NM_000684.3 mRNA, has a length of 3039 bases.

siRNAs comprising a GalNAc conjugate targeting ligand were designed and synthesized as described below. siRNAs comprising an unsaturated C₂₂ hydrocarbon chain conjugated to the sense strand at position 6, counting from the 5′-end of the sense strand, were designed and synthesized as described above.

Detailed lists of the modified ADRB1 sense and antisense strand nucleotide sequences comprising an unsaturated C₂₂ hydrocarbon chain conjugated to the sense strand at position 6, counting from the 5′-end of the sense strand, are shown in Table 3, and the corresponding unmodified ADRB1 sense and antisense nucleotide sequences are shown in Table 2.

Detailed lists of the modified ADRB1 sense and antisense strand nucleotide sequences comprising a GalNAc conjugate targeting ligand are shown in Table 5, and the corresponding unmodified ADRB1 sense and antisense nucleotide sequences are shown in Table 4. It is to be understood that, throughout the application, a duplex name without a decimal is equivalent to a duplex name with a decimal which merely references the batch number of the duplex. For example, AD-959917 is equivalent to AD-959917.1.

siRNA Synthesis

siRNAs comprising a GalNAc conjugate targeting ligand were designed, synthesized, and prepared using methods known in the art.

Briefly, siRNA sequences were synthesized on a 1 μmol scale using a Mermade 192 synthesizer (BioAutomation) with phosphoramidite chemistry on solid supports. The solid support was controlled pore glass (500-1000 A) loaded with a custom GalNAc ligand (3′-GalNAc conjugates), universal solid support (AM Chemicals), or the first nucleotide of interest. Ancillary synthesis reagents and standard 2-cyanoethyl phosphoramidite monomers (2′-deoxy-2′-fluoro, 2′-O-methyl, RNA, DNA) were obtained from Thermo-Fisher (Milwaukee, WI), Hongene (China), or Chemgenes (Wilmington, MA, USA). Additional phosphoramidite monomers were procured from commercial suppliers, prepared in-house, or procured using custom synthesis from various CMOs. Phosphoramidites were prepared at a concentration of 100 mM in either acetonitrile or 9:1 acetonitrile:DMF and were coupled using 5-Ethylthio-1H-tetrazole (ETT, 0.25 M in acetonitrile) with a reaction time of 400 s. Phosphorothioate linkages were generated using a 100 mM solution of 3-((Dimethylamino-methylidene) amino)-3H-1,2,4-dithiazole-3-thione (DDTT, obtained from Chemgenes (Wilmington, MA, USA)) in anhydrous acetonitrile/pyridine (9:1 v/v). Oxidation time was 5 minutes. All sequences were synthesized with final removal of the DMT group (“DMT-Off”). Upon completion of the solid phase synthesis, solid-supported oligoribonucleotides were treated with 300 μL of Methylamine (40% aqueous) at room temperature in 96 well plates for approximately 2 hours to afford cleavage from the solid support and subsequent removal of all additional base-labile protecting groups. For sequences containing any natural ribonucleotide linkages (2′-OH) protected with a tert-butyl dimethyl silyl (TBDMS) group, a second deprotection step was performed using TEA.3HF (triethylamine trihydrofluoride). To each oligonucleotide solution in aqueous methylamine was added 200 μL of dimethyl sulfoxide (DMSO) and 300 μL TEA.3HF and the solution was incubated for approximately 30 mins at 60° C. After incubation, the plate was allowed to come to room temperature and crude oligonucleotides were precipitated by the addition of 1 mL of 9:1 acetontrile:ethanol or 1:1 ethanol:isopropanol. The plates were then centrifuged at 4° C. for 45 mins and the supernatant carefully decanted with the aid of a multichannel pipette. The oligonucleotide pellet was resuspended in 20 mM NaOAc and subsequently desalted using a HiTrap size exclusion column (5 mL, GE Healthcare) on an Agilent LC system equipped with an autosampler, UV detector, conductivity meter, and fraction collector. Desalted samples were collected in 96 well plates and then analyzed by LC-MS and UV spectrometry to confirm identity and quantify the amount of material, respectively.

Duplexing of single strands was performed on a Tecan liquid handling robot. Sense and antisense single strands were combined in an equimolar ratio to a final concentration of 10 μM in 1× PBS in 96 well plates, the plate sealed, incubated at 100° C. for 10 minutes, and subsequently allowed to return slowly to room temperature over a period of 2-3 hours. The concentration and identity of each duplex was confirmed and then subsequently utilized for in vitro screening assays.

### Example 3. In Vitro Screening Methods

### Cell Culture and 384-Well Transfections

Hep3b cells (ATCC, Manassas, VA) were grown to near confluence at 37° C. in an atmosphere of 5% CO₂ in Eagle's Minimum Essential Medium (Gibco) supplemented with 10% FBS (ATCC) before being released from the plate by trypsinization. Transfection was carried out by adding 7.5 l of Opti-MEM plus 0.1 μl of Lipofectamine RNAiMax per well (Invitrogen, Carlsbad CA. cat #13778-150) to 2.5 μl of each siRNA duplex to an individual well in a 384-well plate. The mixture was then incubated at room temperature for 15 minutes. Forty μl of complete growth media without antibiotic containing ˜1.5×10⁴ cells were then added to the siRNA mixture. Cells were incubated for 24 hours prior to RNA purification. Single dose experiments were performed at 10 nM, 1 nM, and 0.1 nM final duplex concentration.

### In Vitro Dual-Luciferase and Endogenous Screening Assays

Hepa1-6 cells were transfected by adding 50 μL of siRNA duplexes and 75 ng of a plasmid, comprising human ADRB1 target sequence, per well along with 100 μL of Opti-MEM plus 0.5 μL of Lipofectamine 2000 per well (Invitrogen, Carlsbad CA. cat #13778-150) and then incubated at room temperature for 15 minutes. The mixture was then added to the cells which are re-suspended in 35 μL of fresh complete media. The transfected cells were incubated at 37° C. in an atmosphere of 5% CO2. Single-dose experiments were performed at 10 nM or 50 nM.

Twenty-four hours after the siRNAs and psiCHECK2 plasmid are transfected; Firefly (transfection control) and Renilla (fused to ADRB1 target sequence) luciferase were measured. First, media was removed from cells. Then Firefly luciferase activity was measured by adding 75 μL of Dual-Glo® Luciferase Reagent equal to the culture medium volume to each well and mix. The mixture was incubated at room temperature for 30 minutes before luminescense (500 nm) was measured on a Spectramax (Molecular Devices) to detect the Firefly luciferase signal. Renilla luciferase activity was measured by adding 75 μL of room temperature of Dual-Glo® Stop & Glo® Reagent to each well and the plates were incubated for 10-15 minutes before luminescence was again measured to determine the Renilla luciferase signal. The Dual-Glo® Stop & Glo® Reagent quenches the firefly luciferase signal and sustained luminescence for the Renilla luciferase reaction. siRNA activity was determined by normalizing the Renilla (MUC5B) signal to the Firefly (control) signal within each well. The magnitude of siRNA activity was then assessed relative to cells that were transfected with the same vector but were not treated with siRNA or were treated with a non-targeting siRNA. All transfections were done with n=4.

Total RNA Isolation Using DYNABEADS mRNA Isolation Kit (Invitrogen™, Part #: 610-12)

Cells were lysed in 75 μl of Lysis/Binding Buffer containing 3 μL of beads per well and mixed for 10 minutes on an electrostatic shaker. The washing steps were automated on a Biotek EL406, using a magnetic plate support. Beads were washed (in 90 L) once in Buffer A, once in Buffer B, and twice in Buffer E, with aspiration steps in between. Following a final aspiration, complete 10 μL RT mixture was added to each well, as described below.

cDNA Synthesis Using AB High Capacity cDNA Reverse Transcription Kit (Applied Biosystems, Foster City, CA, Cat #4368813)

A master mix of 1 μl 10× Buffer, 0.4 μl 25× dNTPs, 1 μl Random primers, 0.5 μl Reverse Transcriptase, 0.5 μl RNase inhibitor and 6.6 μl of H₂O per reaction were added per well. Plates were sealed, agitated for 10 minutes on an electrostatic shaker, and then incubated at 37 degrees C. for 2 hours. Following this, the plates were agitated at 80 degrees C. for 8 minutes.

### Real Time PCR

Two microlitre (μl) of cDNA were added to a master mix containing 0.5 μl of human GAPDH TaqMan Probe (4326317E), 0.5 μl human AGT, 2 μl nuclease-free water and 5 μl Lightcycler 480 probe master mix (Roche Cat #04887301001) per well in a 384 well plates (Roche cat #04887301001). Real time PCR was done in a LightCycler480 Real Time PCR system (Roche). To calculate relative fold change, data were analyzed using the ΔΔCt method and normalized to assays performed with cells transfected with 10 nM AD-1955, or mock transfected cells. IC₅₀s were calculated using a 4 parameter fit model using XLFit and normalized to cells transfected with AD-1955 or mock-transfected. The sense and antisense sequences of AD-1955 are: sense: 5′-cuuAcGcuGAGuAcuucGAdTsdT-3′ and antisense 5′-UCGAAGuACUcAGCGuAAGdTsdT-3′.

The results of the dual-luciferase assays of the agents listed in Tables 4 and 5 are provided in Table 6.

TABLE 1
Abbreviations of nucleotide monomers used in nucleic acid sequence representation. It will
be understood that these monomers, when present in an oligonucleotide, are mutually linked by 5′-3′-
phosphodiester bonds; and it is understood that when the nucleotide contains a 2′-fluoro modification,
then the fluoro replaces the hydroxy at that position in the parent nucleotide (i.e., it is a 2′-deoxy-2′-
fluoronucleotide). It is to be further understood that the nucleotide abbreviations in the table omit the
3′-phosphate (i.e., they are 3′-OH) when placed at the 3′-terminal position of an oligonucleotide.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| CS | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L10 | 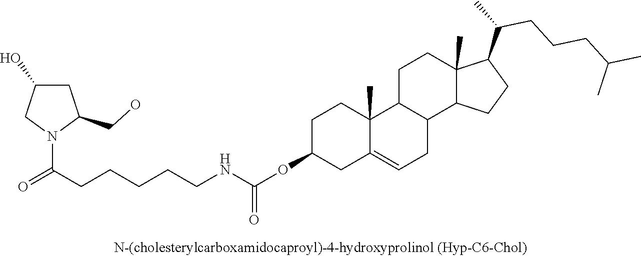 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| L96 | 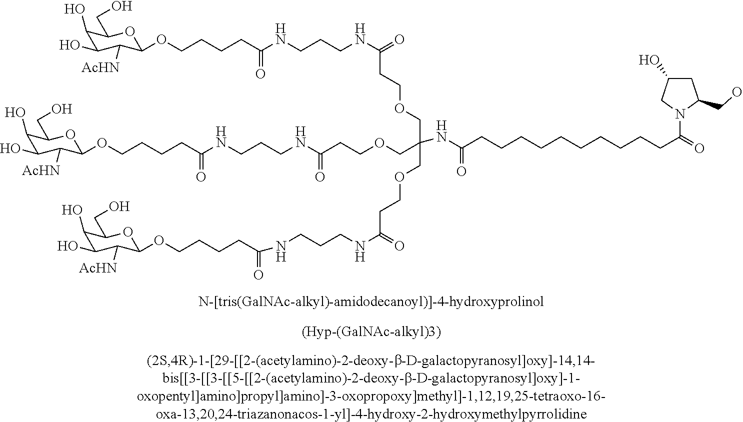 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| uL96 | 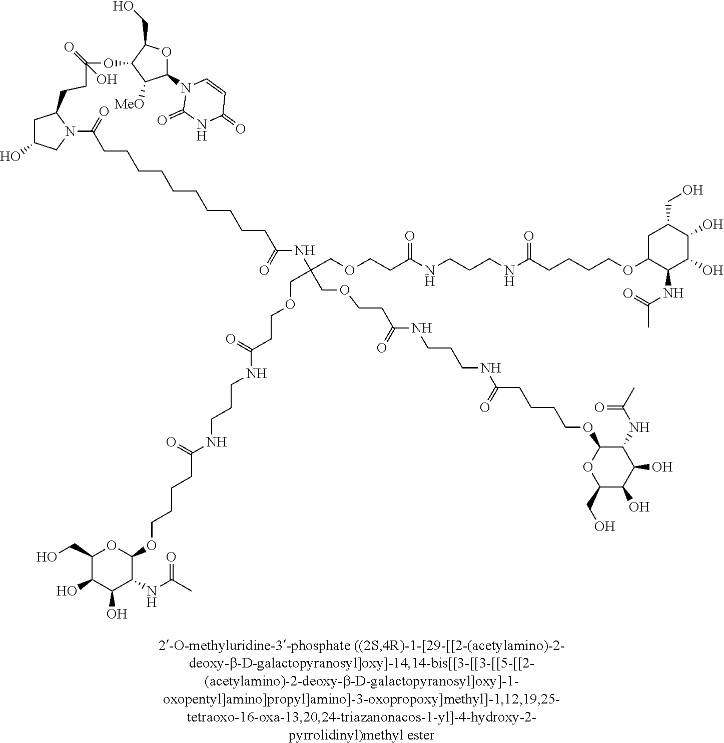 |
| Y34 | 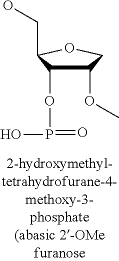 |
| Y44 | 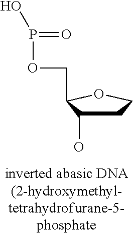 |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythimidine-3′-phosphate |
| dTs | 2′-deoxythimidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Chd) | 2′-O-hexadecyl-cytidine-3′-phosphate |
| (Ahd) | 2′-O-hexadecyl-adenosine-3′-phosphate |
| (Ghd) | 2′-O-hexadecyl-guanosine-3′-phosphate |
| (Uhd) | 2′-O-hexadecyl-uridine-3′-phosphate |
| Ada | 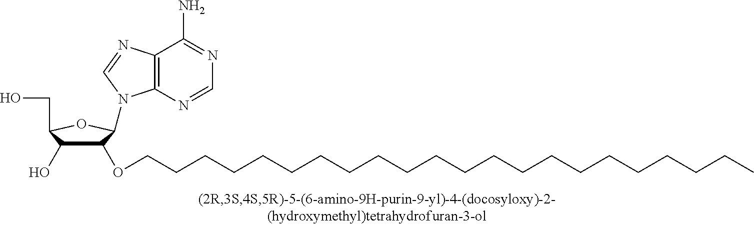 |
| Cda | 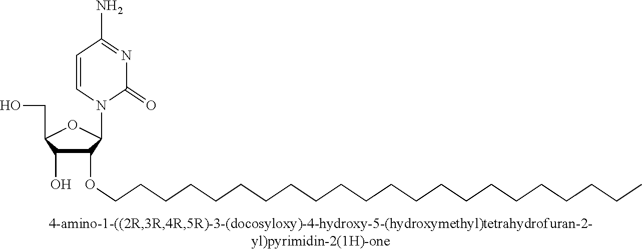 |
| Gda | 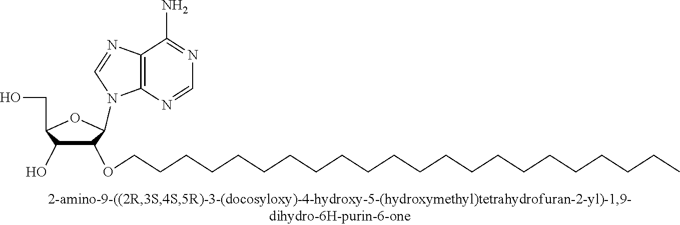 |
| Uda | 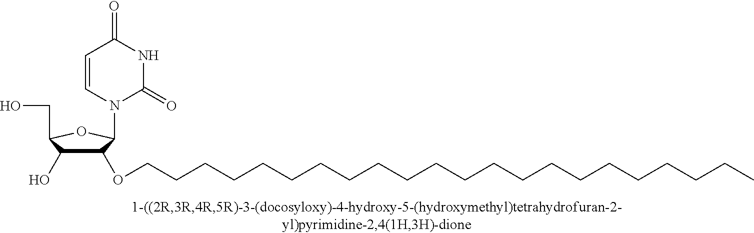 |

TABLE 2
Unmodified Sense and Antisense Strand Sequences of ADRB1 dsRNA Agents

| | | SEQ ID | | Seq ID | Range in |
|---|---|---|---|---|---|
| Duplex Name | Sense Sequence 5′ to 3′ | NO: | Antisense Sequence 5′ to 3′ | NO: | NM_000684.3 |
| AD-1646600 | UAAAUUCUACUUCCUCUUGUA | | UACAAGAGGAAGUAGAAUUUACA | | 2932-2954 |
| AD-1646601 | AUUCAAACUCUACUUCUGUUA | | UAACAGAAGUAGAGUUUGAAUUU | | 2740-2762 |
| AD-1646602 | UCCUUUAUCAUGGUACUGUAA | | UUACAGUACCAUGAUAAAGGAGU | | 2811-2833 |
| AD-1646603 | AAGAAAUGUUAAGCUCUUCUA | | UAGAAGAGCUUAACAUUUCUUGA | | 2301-2323 |
| AD-1646604 | CUUUUUAUACUCCUUUAUCAA | | UUGAUAAAGGAGUAUAAAAAGGU | | 2801-2823 |
| AD-1646605 | AUUGGGAAAAUACCUUUUUAA | | UUAAAAAGGUAUUUUCCCAAUGA | | 2788-2810 |
| AD-1646606 | AAGACAUAUUUCCUUUUGCUA | | UAGCAAAAGGAAAUAUGUCUUGA | | 2184-2206 |
| AD-1646607 | CUACUUCUGUUGUCUAGUAUA | | UAUACUAGACAACAGAAGUAGAG | | 2749-2771 |
| AD-1646608 | AGUGUUAGGAAUUACAAAAUA | | UAUUUUGUAAUUCCUAACACUGU | | 2643-2665 |
| AD-1646609 | AUCACGUUUCAAGAAAUGUUA | | UAACAUUUCUUGAAACGUGAUUU | | 2291-2313 |
| AD-1646610 | GUGGUUCAAAAUGCCAUUUUA | | UAAAAUGGCAUUUUGAACCACUC | | 2618-2640 |
| AD-1646611 | CUUGUUUAUAUUAAACAGCUA | | UAGCUGUUUAAUAUAAACAAGUC | | 2441-2463 |
| AD-1646612 | GUGUUUACUUAAGACCGAUAA | | UUAUCGGUCUUAAGUAAACACAG | | 1756-1778 |
| AD-1646613 | UUCCAGAGAAAUUUCAUUUUA | | UAAAAUGAAAUUUCUCUGGAAAG | | 2204-2226 |
| AD-1646614 | UGUAACUGUAUCCAUAUUAUA | | UAUAAUAUGGAUACAGUUACAGU | | 2827-2849 |
| AD-1646615 | UGCUGUUAUGAAAGCAAAGAA | | UUCUUUGCUUUCAUAACAGCAGA | | 2244-2266 |
| AD-1646616 | UAGUAUGUUAUUGAGCUAAUA | | UAUUAGCUCAAUAACAUACUAGA | | 2763-2785 |
| AD-1646617 | UUUACCAAGUAUUUAUACUCA | | UGAGUAUAAAUACUUGGUAAAAC | | 2956-2978 |
| AD-1646618 | CCUCUUCGUCUUCUUCAACUA | | UAGUUGAAGAAGACGAAGAGGCG | | 1336-1358 |
| AD-1646619 | GUGCAAUAAAUACCAAUGAAA | | UUUCAUUGGUAUUUAUUGCACAU | | 3012-3034 |
| AD-1646620 | CUUGUGUGUUUUACCAAGUAA | | UUACUUGGUAAAACACACAAGAG | | 2947-2969 |
| AD-1646621 | CACCAACCUCUUCAUCAUGUA | | UACAUGAUGAAGAGGUUGGUGAG | | 541-563 |
| AD-1646622 | AGUAAUGAUUUCUGCUGUUAA | | UUAACAGCAGAAAUCAUUACUUA | | 2232-2254 |
| AD-1646623 | GGUGGUUUUUGACACUCUCUA | | UAGAGAGUGUCAAAAACCACCUG | | 2022-2044 |
| AD-1646624 | UGUGACAUGUGACUCUGUCAA | | UUGACAGAGUCACAUGUCACAGA | | 2510-2532 |
| AD-1646625 | UGCCAUUUUUGCACAGUGUUA | | UAACACUGUGCAAAAAUGGCAUU | | 2629-2651 |
| AD-1646626 | UAACUACUGUGUGAGGAAUUA | | UAAUUCCUCACACAGUAGUUAGU | | 2985-3007 |
| AD-1646627 | ACUUGUGUGUAAAUUCUACUA | | UAGUAGAAUUUACACACAAGUGC | | 2923-2945 |
| AD-1646628 | GAUAGAAAGACUUGUUUAUAA | | UUAUAAACAAGUCUUUCUAUCUG | | 2431-2453 |
| AD-1646629 | AAGCUCUUCUUGGAACAAGCA | | UGCUUGUUCCAAGAAGAGCUUAA | | 2311-2333 |
| AD-1646630 | CCACAGAAGAUGUUACUUGCA | | UGCAAGUAACAUCUUCUGUGGAU | | 2663-2685 |
| AD-1646631 | UCUGUCAAUUGAAGACAGGAA | | UUCCUGUCUUCAAUUGACAGAGU | | 2523-2545 |
| AD-1646632 | GAAACAGUUCAGAUUACUGCA | | UGCAGUAAUCUGAACUGUUUCUC | | 2565-2587 |
| AD-1646633 | AUUGAGCUAAUGAUUCAUUGA | | UCAAUGAAUCAUUAGCUCAAUAA | | 2772-2794 |
| AD-1646634 | CCUUUUGCUUUCCAGAGAAAA | | UUUUCUCUGGAAAGCAAAAGGAA | | 2195-2217 |
| AD-1646635 | GUGAUGCAUCUUUAGAUUUUA | | UAAAAUCUAAAGAUGCAUCACAC | | 1990-2012 |
| AD-1646636 | AAUCCUCGUCUGAAUCAUCCA | | UGGAUGAUUCAGACGAGGAUUGU | | 1797-1819 |
| AD-1646637 | AAAGUUAGCACUUGUGUGUAA | | UUACACACAAGUGCUAACUUUCA | | 2914-2936 |
| AD-1646638 | AAUUACAAAAUCCACAGAAGA | | UCUUCUGUGGAUUUUGUAAUUCC | | 2652-2674 |
| AD-1646639 | AUGACAGUUUGUCAAGACAUA | | UAUGUCUUGACAAACUGUCAUUC | | 2171-2193 |
| AD-1646640 | GGGCAGAUCUUAAAUAAAAUA | | UAUUUUAUUUAAGAUCUGCCCAG | | 2722-2744 |
| AD-1646641 | GAUUAAAAUCGAUCAUCGUGA | | UCACGAUGAUCGAUUUUAAUCCC | | 2098-2120 |
| AD-1646642 | UUGCUGAUGUUCCUUGUUGUA | | UACAACAAGGAACAUCAGCAAGC | | 1884-1906 |
| AD-1646643 | CUACCUCACACUGUGCAUUUA | | UAAAUGCACAGUGUGAGGUAGAA | | 2403-2425 |
| AD-1646644 | GUAUCAAUAUUAGUUGGAAGA | | UCUUCCAACUAAUAUUGAUACAU | | 2468-2490 |
| AD-1646645 | AGAUUACUGCACAUGUGGAUA | | UAUCCACAUGUGCAGUAAUCUGA | | 2575-2597 |
| AD-1646646 | GAGGGAUUUCUACCUCACACA | | UGUGUGAGGUAGAAAUCCCUCAG | | 2394-2416 |
| AD-1646647 | UGAAGACAGGACAUUAAAAGA | | UCUUUUAAUGUCCUGUCUUCAAU | | 2532-2554 |
| AD-1646648 | GAGGAGAUCUGUGUUUACUUA | | UAAGUAAACACAGAUCUCCUCGU | | 1746-1768 |
| AD-1646649 | ACUCUGGUGCAACUAACUACA | | UGUAGUUAGUUGCACCAGAGUAU | | 2972-2994 |
| AD-1646650 | CACCUUGCUUUCCUUGUGUAA | | UUACACAAGGAAAGCAAGGUGGG | | 2333-2355 |
| AD-1646651 | UCCGUAGUCUCCUUCUACGUA | | UACGUAGAAGGAGACUACGGACG | | 950-972 |
| AD-1646652 | UUGCUGGUGAAAGUUAGCACA | | UGUGCUAACUUUCACCAGCAAAU | | 2905-2927 |
| AD-1646653 | CUCCUUCUUCUGCGAGCUGUA | | UACAGCUCGCAGAAGAAGGAGCC | | 646-668 |
| AD-1646654 | GGAAUUGGUCCAUGUGCAAUA | | UAUUGCACAUGGACCAAUUCCUC | | 2999-3021 |
| AD-1646655 | UGAAUCAUCCGAGGCAAAGAA | | UUCUUUGCCUCGGAUGAUUCAGA | | 1807-1829 |
| AD-1646656 | AAAGCAAAGAGAAAGGAUGGA | | UCCAUCCUUUCUCUUUGCUUUCA | | 2254-2276 |
| AD-1646657 | UCUUUUGUGUGUGCGUGUGAA | | UUCACACGCACACACAAAAGAAG | | 1974-1996 |
| AD-1646658 | GCAUUUGCACAGCAGAUAGAA | | UUCUAUCUGCUGUGCAAAUGCAC | | 2417-2439 |
| AD-1646659 | AGGAAAGUUUGGGAAGGGAUA | | UAUCCCUUCCCAAACUUUCCUUU | | 1853-1875 |
| AD-1646660 | AGCCUCUCUCUGUGACAUGUA | | UACAUGUCACAGAGAGAGGCUCU | | 2500-2522 |
| AD-1646661 | GGGCGUCUUCACGCUCUGCUA | | UAGCAGAGCGUGAAGACGCCCAU | | 1255-1277 |
| AD-1646662 | AGAAAGGAUGGAGGCAAAAUA | | UAUUUUGCCUCCAUCCUUUCUCU | | 2263-2285 |
| AD-1646663 | GACAUUAAAAGAGAGCGAGAA | | UUCUCGCUCUCUUUUAAUGUCCU | | 2541-2563 |
| AD-1646664 | CGGGAACAGGAACACACUACA | | UGUAGUGUGUUCCUGUUCCCGGG | | 2134-2156 |
| AD-1646665 | AGAGAGGAGAAUGACAGUUUA | | UAAACUGUCAUUCUCCUCUCUCU | | 2161-2183 |
| AD-1646666 | CUGCGACUUCGUCACCAACCA | | UGGUUGGUGACGAAGUCGCAGCA | | 910-932 |
| AD-1646667 | GCCCUUCUUCCUGGCCAACGA | | UCGUUGGCCAGGAAGAAGGGCAG | | 1279-1301 |
| AD-1646668 | UAAGACCGAUAGCAGGUGAAA | | UUUCACCUGCUAUCGGUCUUAAG | | 1765-1787 |
| AD-1646669 | GGUGAAGAAGAUCGACAGCUA | | UAGCUGUCGAUCUUCUUCACCUG | | 1027-1049 |
| AD-1646670 | CACGUGAAUUUGCUGGUGAAA | | UUUCACCAGCAAAUUCACGUGGG | | 2896-2918 |
| AD-1646671 | AGCGAGAGAGAGAAACAGUUA | | UAACUGUUUCUCUCUCUCGCUCU | | 2554-2576 |
| AD-1646672 | GAGGCAAAGAGAAAAGCCACA | | UGUGGCUUUUCUCUUUGCCUCGG | | 1817-1839 |
| AD-1646673 | GCCCAUCCUCAUGCACUGGUA | | UACCAGUGCAUGAGGAUGGGCAG | | 841-863 |
| AD-1646674 | ACCCUGUGUGUCAUUGCCCUA | | UAGGGCAAUGACACACAGGGUCU | | 707-729 |
| AD-1646675 | UAGCAGGUGAACUCGAAGCCA | | UGGCUUCGAGUUCACCUGCUAUC | | 1774-1796 |
| AD-1646676 | GCGCUCAUCGUGCUGCUCAUA | | UAUGAGCAGCACGAUGAGCGCCA | | 461-483 |
| AD-1646677 | CUGUGCAUCAUGGCCUUCGUA | | UACGAAGGCCAUGAUGCACAGGG | | 974-996 |
| AD-1646678 | CCCAUCCCUUUCCCGGGAACA | | UGUUCCCGGGAAAGGGAUGGGAG | | 2121-2143 |
| AD-1646679 | CCUCGGAAUCCAAGGUGUAGA | | UCUACACCUUGGAUUCCGAGGCG | | 1680-1702 |
| AD-1646680 | GAAGCCCACAAUCCUCGUCUA | | UAGACGAGGAUUGUGGGCUUCGA | | 1788-1810 |
| AD-1646681 | AUGGCCUUCGUGUACCUGCGA | | UCGCAGGUACACGAAGGCCAUGA | | 983-1005 |
| AD-1646682 | UUAUGUCCAAGUGCCCACGUA | | UACGUGGGCACUUGGACAUAAAA | | 2881-2903 |
| AD-1646683 | GACACUCUCUGAGAGGACCGA | | UCGGUCCUCUCAGAGAGUGUCAA | | 2032-2054 |
| AD-1646684 | GACGACGACGAUGUCGUCGGA | | UCCGACGACAUCGUCGUCGUCGU | | 1556-1578 |
| AD-1646685 | AUCGUGGCUCCCAUCCCUUUA | | UAAAGGGAUGGGAGCCACGAUGA | | 2112-2134 |
| AD-1646686 | CUGGUGUCCUUCCUGCCCAUA | | UAUGGGCAGGAAGGACACCAGGG | | 827-849 |
| AD-1646687 | AAGGGAGAAGCAUUAGGAGGA | | UCCUCCUAAUGCUUCUCCCUUCC | | 2077-2099 |
| AD-1646688 | CGCUGUCUCAGCAGUGGACAA | | UUGUCCACUGCUGAGACAGCGGC | | 420-442 |
| AD-1646689 | UACGCCAACUCGGCCUUCAAA | | UUUGAAGGCCGAGUUGGCGUAGC | | 1364-1386 |
| AD-1646690 | CUCGCCAUCACCUCGCCCUUA | | UAAGGGCGAGGUGAUGGCGAGGU | | 737-759 |
| AD-1646691 | GUCCGGCCUUCUUUUGUGUGA | | UCACACAAAAGAAGGCCGGACCA | | 1965-1987 |
| AD-1646692 | AUCGCCUCGUCCGUAGUCUCA | | UGAGACUACGGACGAGGCGAUGG | | 941-963 |
| AD-1646693 | CGGGCAAUGUGCUGGUGAUCA | | UGAUCACCAGCACAUUGCCCGCC | | 486-508 |
| AD-1646694 | CUUCUUCAACUGGCUGGGCUA | | UAGCCCAGCCAGUUGAAGAAGAC | | 1345-1367 |
| AD-1646695 | CUUGUGUAGGGCAAACCCGCA | | UGCGGGUUUGCCCUACACAAGGA | | 2345-2367 |
| AD-1646696 | GGCCAACGUGGUGAAGGCCUA | | UAGGCCUUCACCACGUUGGCCAG | | 1291-1313 |
| AD-1646697 | GGAGUACGGCUCCUUCUUCUA | | UAGAAGAAGGAGCCGUACUCCCA | | 637-659 |
| AD-1646698 | GCCCAGAAGCAGGUGAAGAAA | | UUUCUUCACCUGCUUCUGGGCCU | | 1016-1038 |
| AD-1646699 | CAGAAGGCGCUCAAGACGCUA | | UAGCGUCUUGAGCGCCUUCUGCU | | 1223-1245 |
| AD-1646700 | UUCCGCUACCAGAGCCUGCUA | | UAGCAGGCUCUGGUAGCGGAAGG | | 755-777 |
| AD-1646701 | CUUCCGCAAGGCCUUCCAGGA | | UCCUGGAAGGCCUUGCGGAAGUC | | 1411-1433 |
| AD-1646702 | GAGUGGAAGAUGGGUGGGUUA | | UAACCCACCCAUCUUCCACUCCG | | 2052-2074 |
| AD-1646703 | GGCCAGCAUCGAGACCCUGUA | | UACAGGGUCUCGAUGCUGGCCGU | | 694-716 |
| AD-1646704 | CUGCUGGCUGCCCUUCUUCCA | | UGGAAGAAGGGCAGCCAGCAGAG | | 1270-1292 |
| AD-1646705 | GACCGCUACCUCGCCAUCACA | | UGUGAUGGCGAGGUAGCGGUCCA | | 728-750 |
| AD-1646706 | CGUGGCCAUCGCCAAGACGCA | | UGCGUCUUGGCGAUGGCCACGAU | | 505-527 |
| AD-1646707 | AUGGGUCUGCUGAUGGCGCUA | | UAGCGCCAUCAGCAGACCCAUGC | | 446-468 |
| AD-1646708 | GGGCAUCAUCAUGGGCGUCUA | | UAGACGCCCAUGAUGAUGCCCAG | | 1243-1265 |
| AD-1646709 | GAACACACUACCAGCCAGAGA | | UCUCUGGCUGGUAGUGUGUUCCU | | 2143-2165 |
| AD-1646710 | AUUGCCCUGGACCGCUACCUA | | UAGGUAGCGGUCCAGGGCAAUGA | | 719-741 |
| AD-1646711 | AUCUCGGCCCUGGUGUCCUUA | | UAAGGACACCAGGGCCGAGAUGG | | 818-840 |
| AD-1646712 | AGCUGCGAGCGCCGUUUCCUA | | UAGGAAACGGCGCUCGCAGCUGU | | 1043-1065 |
| AD-1646713 | CAUCAUCUACUGCCGCAGCCA | | UGGCUGCGGCAGUAGAUGAUGGG | | 1387-1409 |
| AD-1646714 | GCAGACGCUCACCAACCUCUA | | UAGAGGUUGGUGAGCGUCUGCAG | | 532-554 |
| AD-1646715 | CUUCCAGGGACUGCUCUGCUA | | UAGCAGAGCAGUCCCUGGAAGGC | | 1423-1445 |
| AD-1646716 | CUUCAUCAUGUCCCUGGCCAA | | UUGGCCAGGGACAUGAUGAAGAG | | 550-572 |
| AD-1646717 | AAACAUGCUGAAGUCCCGGCA | | UGCCGGGACUUCAGCAUGUUUCU | | 3-23 |
| AD-1646718 | AAGGCCUUCCACCGCGAGCUA | | UAGCUCGCGGUGGAAGGCCUUCA | | 1304-1326 |
| AD-1646719 | CAGGCGCAGAGCCUCUCUCUA | | UAGAGAGAGGCUCUGCGCCUGGU | | 2491-2513 |
| AD-1646720 | CGAGCCCGGUAACCUGUCGUA | | UACGACAGGUUACCGGGCUCGGA | | 298-320 |
| AD-1646721 | CUGUGGACCUCAGUGGACGUA | | UACGUCCACUGAGGUCCACAGCU | | 662-684 |
| AD-1646722 | GCCACGGACCGUUGCACAAAA | | UUUUGUGCAACGGUCCGUGGCUU | | 1832-1854 |
| AD-1646723 | ACCUCGCCCUUCCGCUACCAA | | UUGGUAGCGGAAGGGCGAGGUGA | | 746-768 |
| AD-1646724 | UCAAGACGCUGGGCAUCAUCA | | UGAUGAUGCCCAGCGUCUUGAGC | | 1233-1255 |
| AD-1646725 | GGAGAGUGGCUUGCUGAUGUA | | UACAUCAGCAAGCCACUCUCCCA | | 1874-1896 |
| AD-1646726 | CUGCUCAUCGUGGCGGGCAAA | | UUUGCCCGCCACGAUGAGCAGCA | | 473-495 |
| AD-1646727 | ACCAGCCAGAGAGAGGAGAAA | | UUUCUCCUCUCUCUGGCUGGUAG | | 2152-2174 |
| AD-1646728 | GCUGGUGAUCGUGGCCAUCGA | | UCGAUGGCCACGAUCACCAGCAC | | 496-518 |
| AD-1646729 | AAGUCCCGGCGGCUCUUCCAA | | UUGGAAGAGCCGCCGGGACUUCA | | 13-33 |
| AD-1646730 | GGUCAGGCUGAGGGAUUUCUA | | UAGAAAUCCCUCAGCCUGACCAC | | 2385-2407 |
| AD-1646731 | UCAGUGGACGUGCUGUGCGUA | | UACGCACAGCACGUCCACUGAGG | | 671-693 |
| AD-1646732 | UACCUGCGGGUGUUCCGCGAA | | UUCGCGGAACACCCGCAGGUACA | | 995-1017 |
| AD-1646733 | UUUCUGUUUGUGGUCCGGCCA | | UGGCCGGACCACAAACAGAAAAA | | 1953-1975 |
| AD-1646734 | ACCAACCGGGCCUACGCCAUA | | UAUGGCGUAGGCCCGGUUGGUGA | | 923-945 |

TABLE 3
Modified Sense and Antisense Strand Sequences of ADRB1 dsRNA Agents Comprising an Unsaturated C₂₂
Hydrocarbon Chain Conjugated to Position 6 on the Sense Strand, Counting from the 5′-end of the
Sense Strand

| | | SEQ | | SEQ | | SEQ |
|---|---|---|---|---|---|---|
| Duplex | | ID | | ID | mRNA Target Sequence | ID |
| Name | Sense Sequence 5′ to 3′ | NO: | Antisense Sequence 5′ to 3′ | NO: | 5′ to 3′ | NO: |
| AD- | usasaau(Uda)CfuAfCfUfuccucuugs | | VPusAfscaaGfaGfGfaaguAfgAfauuua | | UGUAAAUUCUACUUCCUCUU | |
| 1646600 | usa | | scsa | | GUG | |
| AD- | asusuca(Ada)AfcUfCfUfacuucugus | | VPusAfsacaGfaAfGfuagaGfuUfugaau | | AAAUUCAAACUCUACUUCUG | |
| 1646601 | usa | | susu | | UUG | |
| AD- | uscscuu(Uda)AfuCfAfUfgguacugus | | VPusUfsacaGfuAfCfcaugAfuAfaagga | | ACUCCUUUAUCAUGGUACUG | |
| 1646602 | asa | | sgsu | | UAA | |
| AD- | asasgaa(Ada)UfgUfUfAfagcucuucs | | VPusAfsgaaGfaGfCfuuaaCfaUfuucuu | | UCAAGAAAUGUUAAGCUCUU | |
| 1646603 | usa | | sgsa | | CUU | |
| AD- | csusuuu(Uda)AfuAfCfUfccuuuaucs | | VPusUfsgauAfaAfGfgaguAfuAfaaaag | | ACCUUUUUAUACUCCUUUAU | |
| 1646604 | asa | | sgsu | | CAU | |
| AD- | asusugg(Gda)AfaAfAfUfaccuuuuus | | VPusUfsaaaAfaGfGfuauuUfuCfccaaus | | UCAUUGGGAAAAUACCUUUU | |
| 1646605 | asa | | gsa | | UAU | |
| AD- | asasgac(Ada)UfaUfUfUfccuuuugcs | | VPusAfsgcaAfaAfGfgaaaUfaUfgucuu | | UCAAGACAUAUUUCCUUUUG | |
| 1646606 | usa | | sgsa | | CUU | |
| AD- | csusacu(Uda)CfuGfUfUfgucuaguas | | VPusAfsuacUfaGfAfcaacAfgAfaguag | | CUCUACUUCUGUUGUCUAGU | |
| 1646607 | usa | | sasg | | AUG | |
| AD- | asgsugu(Uda)AfgGfAfAfuuacaaaas | | VPusAfsuuuUfgUfAfauucCfuAfacacu | | ACAGUGUUAGGAAUUACAAA | |
| 1646608 | usa | | sgsu | | AUC | |
| AD- | asuscac(Gda)UfuUfCfAfagaaaugus | | VPusAfsacaUfuUfCfuugaAfaCfgugau | | AAAUCACGUUUCAAGAAAUG | |
| 1646609 | usa | | susu | | UUA | |
| AD- | gsusggu(Uda)CfaAfAfAfugccauuus | | VPusAfsaaaUfgGfCfauuuUfgAfaccacs | | GAGUGGUUCAAAAUGCCAUU | |
| 1646610 | usa | | usc | | UUU | |
| AD- | csusugu(Uda)UfaUfAfUfuaaacagcs | | VPusAfsgcuGfuUfUfaauaUfaAfacaag | | GACUUGUUUAUAUUAAACAG | |
| 1646611 | usa | | susc | | CUU | |
| AD- | gsusguu(Uda)AfcUfUfAfagaccgaus | | VPusUfsaucGfgUfCfuuaaGfuAfaacacs | | CUGUGUUUACUUAAGACCGA | |
| 1646612 | asa | | asg | | UAG | |
| AD- | ususcca(Gda)AfgAfAfAfuuucauuus | | VPusAfsaaaUfgAfAfauuuCfuCfuggaa | | CUUUCCAGAGAAAUUUCAUU | |
| 1646613 | usa | | sasg | | UUA | |
| AD- | usgsuaa(Cda)UfgUfAfUfccauauuas | | VPusAfsuaaUfaUfGfgauaCfaGfuuacas | | ACUGUAACUGUAUCCAUAUU | |
| 1646614 | usa | | gsu | | AUA | |
| AD- | usgscug(Uda)UfaUfGfAfaagcaaags | | VPusUfscuuUfgCfUfuucaUfaAfcagca | | UCUGCUGUUAUGAAAGCAAA | |
| 1646615 | asa | | sgsa | | GAG | |
| AD- | usasgua(Uda)GfuUfAfUfugagcuaas | | VPusAfsuuaGfcUfCfaauaAfcAfuacuas | | UCUAGUAUGUUAUUGAGCUA | |
| 1646616 | usa | | gsa | | AUG | |
| AD- | ususuac(Cda)AfaGfUfAfuuuauacus | | VPusGfsaguAfuAfAfauacUfuGfguaaa | | GUUUUACCAAGUAUUUAUAC | |
| 1646617 | csa | | sasc | | UCU | |
| AD- | cscsucu(Uda)CfgUfCfUfucuucaacs | | VPusAfsguuGfaAfGfaagaCfgAfagagg | | CGCCUCUUCGUCUUCUUCAA | |
| 1646618 | usa | | scsg | | CUG | |
| AD- | gsusgca(Ada)UfaAfAfUfaccaaugas | | VPusUfsucaUfuGfGfuauuUfaUfugcac | | AUGUGCAAUAAAUACCAAUG | |
| 1646619 | asa | | sasu | | AAG | |
| AD- | csusugu(Gda)UfgUfUfUfuaccaagus | | VPusUfsacuUfgGfUfaaaaCfaCfacaags | | CUCUUGUGUGUUUUACCAAG | |
| 1646620 | asa | | asg | | UAU | |
| AD- | csascca(Ada)CfcUfCfUfucaucaugs | | VPusAfscauGfaUfGfaagaGfgUfuggug | | CUCACCAACCUCUUCAUCAU | |
| 1646621 | usa | | sasg | | GUC | |
| AD- | asgsuaa(Uda)GfaUfUfUfcugcuguus | | VPusUfsaacAfgCfAfgaaaUfcAfuuacus | | UAAGUAAUGAUUUCUGCUGU | |
| 1646622 | asa | | usa | | UAU | |
| AD- | gsgsugg(Uda)UfuUfUfGfacacucucs | | VPusAfsgagAfgUfGfucaaAfaAfccaccs | | CAGGUGGUUUUUGACACUCU | |
| 1646623 | usa | | usg | | CUG | |
| AD- | usgsuga(Cda)AfuGfUfGfacucugucs | | VPusUfsgacAfgAfGfucacAfuGfucaca | | UCUGUGACAUGUGACUCUGU | |
| 1646624 | asa | | sgsa | | CAA | |
| AD- | usgscca(Uda)UfuUfUfGfcacagugus | | VPusAfsacaCfuGfUfgcaaAfaAfuggcas | | AAUGCCAUUUUUGCACAGUG | |
| 1646625 | usa | | usu | | UUA | |
| AD- | usasacu(Ada)CfuGfUfGfugaggaaus | | VPusAfsauuCfcUfCfacacAfgUfaguuas | | ACUAACUACUGUGUGAGGAA | |
| 1646626 | usa | | gsu | | UUG | |
| AD- | ascsuug(Uda)GfuGfUfAfaauucuacs | | VPusAfsguaGfaAfUfuuacAfcAfcaagu | | GCACUUGUGUGUAAAUUCUA | |
| 1646627 | usa | | sgsc | | CUU | |
| AD- | gsasuag(Ada)AfaGfAfCfuuguuuaus | | VPusUfsauaAfaCfAfagucUfuUfcuauc | | CAGAUAGAAAGACUUGUUUA | |
| 1646628 | asa | | susg | | UAU | |
| AD- | asasgcu(Cda)UfuCfUfUfggaacaags | | VPusGfscuuGfuUfCfcaagAfaGfagcuu | | UUAAGCUCUUCUUGGAACAA | |
| 1646629 | csa | | sasa | | GCC | |
| AD- | cscsaca(Gda)AfaGfAfUfguuacuugs | | VPusGfscaaGfuAfAfcaucUfuCfugugg | | AUCCACAGAAGAUGUUACUU | |
| 1646630 | csa | | sasu | | GCA | |
| AD- | uscsugu(Cda)AfaUfUfGfaagacaggs | | VPusUfsccuGfuCfUfucaaUfuGfacagas | | ACUCUGUCAAUUGAAGACAG | |
| 1646631 | asa | | gsu | | GAC | |
| AD- | gsasaac(Ada)GfuUfCfAfgauuacugs | | VPusGfscagUfaAfUfcugaAfcUfguuuc | | GAGAAACAGUUCAGAUUACU | |
| 1646632 | csa | | susc | | GCA | |
| AD- | asusuga(Gda)CfuAfAfUfgauucauus | | VPusCfsaauGfaAfUfcauuAfgCfucaaus | | UUAUUGAGCUAAUGAUUCAU | |
| 1646633 | gsa | | asa | | UGG | |
| AD- | cscsuuu(Uda)GfcUfUfUfccagagaas | | VPusUfsuucUfcUfGfgaaaGfcAfaaagg | | UUCCUUUUGCUUUCCAGAGA | |
| 1646634 | asa | | sasa | | AAU | |
| AD- | gsusgau(Gda)CfaUfCfUfuuagauuus | | VPusAfsaaaUfcUfAfaagaUfgCfaucacs | | GUGUGAUGCAUCUUUAGAUU | |
| 1646635 | usa | | asc | | UUU | |
| AD- | asasucc(Uda)CfgUfCfUfgaaucaucs | | VPusGfsgauGfaUfUfcagaCfgAfggauu | | ACAAUCCUCGUCUGAAUCAU | |
| 1646636 | csa | | sgsu | | CCG | |
| AD- | asasagu(Uda)AfgCfAfCfuugugugus | | VPusUfsacaCfaCfAfagugCfuAfacuuus | | UGAAAGUUAGCACUUGUGUG | |
| 1646637 | asa | | csa | | UAA | |
| AD- | asasuua(Cda)AfaAfAfUfccacagaas | | VPusCfsuucUfgUfGfgauuUfuGfuaauu | | GGAAUUACAAAAUCCACAGA | |
| 1646638 | gsa | | ScSC | | AGA | |
| AD- | asusgac(Ada)GfuUfUfGfucaagacas | | VPusAfsuguCfuUfGfacaaAfcUfgucau | | GAAUGACAGUUUGUCAAGAC | |
| 1646639 | usa | | susc | | AUA | |
| AD- | gsgsgca(Gda)AfuCfUfUfaaauaaaas | | VPusAfsuuuUfaUfUfuaagAfuCfugccc | | CUGGGCAGAUCUUAAAUAAA | |
| 1646640 | usa | | sasg | | AUU | |
| AD- | gsasuua(Ada)AfaUfCfGfaucaucgus | | VPusCfsacgAfuGfAfucgaUfuUfuaauc | | GGGAUUAAAAUCGAUCAUCG | |
| 1646641 | gsa | | scsc | | UGG | |
| AD- | ususgcu(Gda)AfuGfUfUfccuuguug | | VPusAfscaaCfaAfGfgaacAfuCfagcaas | | GCUUGCUGAUGUUCCUUGUU | |
| 1646642 | susa | | gsc | | GUU | |
| AD- | csusacc(Uda)CfaCfAfCfugugcauus | | VPusAfsaauGfcAfCfagugUfgAfgguag | | UUCUACCUCACACUGUGCAU | |
| 1646643 | usa | | sasa | | UUG | |
| AD- | gsusauc(Ada)AfuAfUfUfaguuggaas | | VPusCfsuucCfaAfCfuaauAfuUfgauacs | | AUGUAUCAAUAUUAGUUGGA | |
| 1646644 | gsa | | asu | | AGG | |
| AD- | asgsauu(Ada)CfuGfCfAfcauguggas | | VPusAfsuccAfcAfUfgugcAfgUfaaucu | | UCAGAUUACUGCACAUGUGG | |
| 1646645 | usa | | sgsa | | AUA | |
| AD- | gsasggg(Ada)UfuUfCfUfaccucacas | | VPusGfsuguGfaGfGfuagaAfaUfcccuc | | CUGAGGGAUUUCUACCUCAC | |
| 1646646 | csa | | sasg | | ACU | |
| AD- | usgsaag(Ada)CfaGfGfAfcauuaaaas | | VPusCfsuuuUfaAfUfguccUfgUfcuuca | | AUUGAAGACAGGACAUUAAA | |
| 1646647 | gsa | | sasu | | AGA | |
| AD- | gsasgga(Gda)AfuCfUfGfuguuuacus | | VPusAfsaguAfaAfCfacagAfuCfuccucs | | ACGAGGAGAUCUGUGUUUAC | |
| 1646648 | usa | | gsu | | UUA | |
| AD- | ascsucu(Gda)GfuGfCfAfacuaacuas | | VPusGfsuagUfuAfGfuugcAfcCfagagu | | AUACUCUGGUGCAACUAACU | |
| 1646649 | csa | | sasu | | ACU | |
| AD- | csasccu(Uda)GfcUfUfUfccuugugus | | VPusUfsacaCfaAfGfgaaaGfcAfaggugs | | CCCACCUUGCUUUCCUUGUG | |
| 1646650 | asa | | gsg | | UAG | |
| AD- | uscscgu(Ada)GfuCfUfCfcuucuacgs | | VPusAfscguAfgAfAfggagAfcUfacgga | | CGUCCGUAGUCUCCUUCUAC | |
| 1646651 | usa | | scsg | | GUG | |
| AD- | ususgcu(Gda)GfuGfAfAfaguuagcas | | VPusGfsugcUfaAfCfuuucAfcCfagcaas | | AUUUGCUGGUGAAAGUUAGC | |
| 1646652 | csa | | asu | | ACU | |
| AD- | csusccu(Uda)CfuUfCfUfgcgagcugs | | VPusAfscagCfuCfGfcagaAfgAfaggag | | GGCUCCUUCUUCUGCGAGCU | |
| 1646653 | usa | | scSc | | GUG | |
| AD- | gsgsaau(Uda)GfgUfCfCfaugugcaas | | VPusAfsuugCfaCfAfuggaCfcAfauucc | | GAGGAAUUGGUCCAUGUGCA | |
| 1646654 | usa | | susc | | AUA | |
| AD- | usgsaau(Cda)AfuCfCfGfaggcaaags | | VPusUfscuuUfgCfCfucggAfuGfauuca | | UCUGAAUCAUCCGAGGCAAA | |
| 1646655 | asa | | sgsa | | GAG | |
| AD- | asasagc(Ada)AfaGfAfGfaaaggaugs | | VPusCfscauCfcUfUfucucUfuUfgcuuu | | UGAAAGCAAAGAGAAAGGAU | |
| 1646656 | gsa | | scsa | | GGA | |
| AD- | uscsuuu(Uda)GfuGfUfGfugcgugug | | VPusUfscacAfcGfCfacacAfcAfaaagas | | CUUCUUUUGUGUGUGCGUGU | |
| 1646657 | sasa | | asg | | GAU | |
| AD- | gscsauu(Uda)GfcAfCfAfgcagauags | | VPusUfscuaUfcUfGfcuguGfcAfaaugc | | GUGCAUUUGCACAGCAGAUA | |
| 1646658 | asa | | sasc | | GAA | |
| AD- | asgsgaa(Ada)GfuUfUfGfggaagggas | | VPusAfsuccCfuUfCfccaaAfcUfuuccus | | AAAGGAAAGUUUGGGAAGGG | |
| 1646659 | usa | | usu | | AUG | |
| AD- | asgsccu(Cda)UfcUfCfUfgugacaugs | | VPusAfscauGfuCfAfcagaGfaGfaggcu | | AGAGCCUCUCUCUGUGACAU | |
| 1646660 | usa | | scsu | | GUG | |
| AD- | gsgsgcg(Uda)CfuUfCfAfcgcucugcs | | VPusAfsgcaGfaGfCfgugaAfgAfcgccc | | AUGGGCGUCUUCACGCUCUG | |
| 1646661 | usa | | sasu | | CUG | |
| AD- | asgsaaa(Gda)GfaUfGfGfaggcaaaas | | VPusAfsuuuUfgCfCfuccaUfcCfuuucu | | AGAGAAAGGAUGGAGGCAAA | |
| 1646662 | usa | | scsu | | AUA | |
| AD- | gsascau(Uda)AfaAfAfGfagagcgags | | VPusUfscucGfcUfCfucuuUfuAfauguc | | AGGACAUUAAAAGAGAGCGA | |
| 1646663 | asa | | scsu | | GAG | |
| AD- | csgsgga(Ada)CfaGfGfAfacacacuas | | VPusGfsuagUfgUfGfuuccUfgUfucccg | | CCCGGGAACAGGAACACACU | |
| 1646664 | csa | | sgsg | | ACC | |
| AD- | asgsaga(Gda)GfaGfAfAfugacaguus | | VPusAfsaacUfgUfCfauucUfcCfucucus | | AGAGAGAGGAGAAUGACAGU | |
| 1646665 | usa | | csu | | UUG | |
| AD- | csusgcg(Ada)CfuUfCfGfucaccaacs | | VPusGfsguuGfgUfGfacgaAfgUfcgcag | | UGCUGCGACUUCGUCACCAA | |
| 1646666 | csa | | scsa | | CCG | |
| AD- | gscsccu(Uda)CfuUfCfCfuggccaacs | | VPusCfsguuGfgCfCfaggaAfgAfagggc | | CUGCCCUUCUUCCUGGCCAA | |
| 1646667 | gsa | | sasg | | CGU | |
| AD- | usasaga(Cda)CfgAfUfAfgcaggugas | | VPusUfsucaCfcUfGfcuauCfgGfucuua | | CUUAAGACCGAUAGCAGGUG | |
| 1646668 | asa | | sasg | | AAC | |
| AD- | gsgsuga(Ada)GfaAfGfAfucgacagcs | | VPusAfsgcuGfuCfGfaucuUfcUfucacc | | CAGGUGAAGAAGAUCGACAG | |
| 1646669 | usa | | susg | | CUG | |
| AD- | csascgu(Gda)AfaUfUfUfgcuggugas | | VPusUfsucaCfcAfGfcaaaUfuCfacgugs | | CCCACGUGAAUUUGCUGGUG | |
| 1646670 | asa | | gsg | | AAA | |
| AD- | asgscga(Gda)AfgAfGfAfgaaacagus | | VPusAfsacuGfuUfUfcucuCfuCfucgcu | | AGAGCGAGAGAGAGAAACAG | |
| 1646671 | usa | | scsu | | UUC | |
| AD- | gsasggc(Ada)AfaGfAfGfaaaagccas | | VPusGfsuggCfuUfUfucucUfuUfgccuc | | CCGAGGCAAAGAGAAAAGCC | |
| 1646672 | csa | | sgsg | | ACG | |
| AD- | gscscca(Uda)CfcUfCfAfugcacuggs | | VPusAfsccaGfuGfCfaugaGfgAfugggc | | CUGCCCAUCCUCAUGCACUG | |
| 1646673 | usa | | sasg | | GUG | |
| AD- | ascsccu(Gda)UfgUfGfUfcauugcccs | | VPusAfsgggCfaAfUfgacaCfaCfagggu | | AGACCCUGUGUGUCAUUGCC | |
| 1646674 | usa | | scsu | | CUG | |
| AD- | usasgca(Gda)GfuGfAfAfcucgaagcs | | VPusGfsgcuUfcGfAfguucAfcCfugcua | | GAUAGCAGGUGAACUCGAAG | |
| 1646675 | csa | | susc | | CCC | |
| AD- | gscsgcu(Cda)AfuCfGfUfgcugcucas | | VPusAfsugaGfcAfGfcacgAfuGfagcgc | | UGGCGCUCAUCGUGCUGCUC | |
| 1646676 | usa | | scsa | | AUC | |
| AD- | csusgug(Cda)AfuCfAfUfggccuucgs | | VPusAfscgaAfgGfCfcaugAfuGfcacag | | CCCUGUGCAUCAUGGCCUUC | |
| 1646677 | usa | | sgsg | | GUG | |
| AD- | cscscau(Cda)CfcUfUfUfcccgggaasc | | VPusGfsuucCfcGfGfgaaaGfgGfauggg | | CUCCCAUCCCUUUCCCGGGA | |
| 1646678 | sa | | sasg | | ACA | |
| AD- | cscsucg(Gda)AfaUfCfCfaagguguas | | VPusCfsuacAfcCfUfuggaUfuCfcgagg | | CGCCUCGGAAUCCAAGGUGU | |
| 1646679 | gsa | | scsg | | AGG | |
| AD- | gsasagc(Cda)CfaCfAfAfuccucgucs | | VPusAfsgacGfaGfGfauugUfgGfgcuuc | | UCGAAGCCCACAAUCCUCGU | |
| 1646680 | usa | | sgsa | | CUG | |
| AD- | asusggc(Cda)UfuCfGfUfguaccugcs | | VPusCfsgcaGfgUfAfcacgAfaGfgccaus | | UCAUGGCCUUCGUGUACCUG | |
| 1646681 | gsa | | gsa | | CGG | |
| AD- | ususaug(Uda)CfcAfAfGfugcccacgs | | VPusAfscguGfgGfCfacuuGfgAfcauaa | | UUUUAUGUCCAAGUGCCCAC | |
| 1646682 | usa | | sasa | | GUG | |
| AD- | gsascac(Uda)CfuCfUfGfagaggaccs | | VPusCfsgguCfcUfCfucagAfgAfguguc | | UUGACACUCUCUGAGAGGAC | |
| 1646683 | gsa | | sasa | | CGG | |
| AD- | gsascga(Cda)GfaCfGfAfugucgucgs | | VPusCfscgaCfgAfCfaucgUfcGfucgucs | | ACGACGACGACGAUGUCGUC | |
| 1646684 | gsa | | gsu | | GGG | |
| AD- | asuscgu(Gda)GfcUfCfCfcaucccuus | | VPusAfsaagGfgAfUfgggaGfcCfacgau | | UCAUCGUGGCUCCCAUCCCU | |
| 1646685 | usa | | sgsa | | UUC | |
| AD- | csusggu(Gda)UfcCfUfUfccugcccas | | VPusAfsuggGfcAfGfgaagGfaCfaccag | | CCCUGGUGUCCUUCCUGCCC | |
| 1646686 | usa | | sgsg | | AUC | |
| AD- | asasggg(Ada)GfaAfGfCfauuaggags | | VPusCfscucCfuAfAfugcuUfcUfcccuu | | GGAAGGGAGAAGCAUUAGGA | |
| 1646687 | gsa | | scSC | | GGG | |
| AD- | csgscug(Uda)CfuCfAfGfcaguggacs | | VPusUfsgucCfaCfUfgcugAfgAfcagcg | | GCCGCUGUCUCAGCAGUGGA | |
| 1646688 | asa | | sgsc | | CAG | |
| AD- | usascgc(Cda)AfaCfUfCfggccuucas | | VPusUfsugaAfgGfCfcgagUfuGfgcgua | | GCUACGCCAACUCGGCCUUC | |
| 1646689 | asa | | sgsc | | AAC | |
| AD- | csuscgc(Cda)AfuCfAfCfcucgcccus | | VPusAfsaggGfcGfAfggugAfuGfgcgag | | ACCUCGCCAUCACCUCGCCC | |
| 1646690 | usa | | sgsu | | UUC | |
| AD- | gsusccg(Gda)CfcUfUfCfuuuugugus | | VPusCfsacaCfaAfAfagaaGfgCfcggacs | | UGGUCCGGCCUUCUUUUGUG | |
| 1646691 | gsa | | csa | | UGU | |
| AD- | asuscgc(Cda)UfcGfUfCfcguagucus | | VPusGfsagaCfuAfCfggacGfaGfgcgau | | CCAUCGCCUCGUCCGUAGUC | |
| 1646692 | csa | | sgsg | | UCC | |
| AD- | csgsggc(Ada)AfuGfUfGfcuggugaus | | VPusGfsaucAfcCfAfgcacAfuUfgcccgs | | GGCGGGCAAUGUGCUGGUGA | |
| 1646693 | csa | | CSC | | UCG | |
| AD- | csusucu(Uda)CfaAfCfUfggcugggcs | | VPusAfsgccCfaGfCfcaguUfgAfagaags | | GUCUUCUUCAACUGGCUGGG | |
| 1646694 | usa | | asc | | CUA | |
| AD- | csusugu(Gda)UfaGfGfGfcaaacccgs | | VPusGfscggGfuUfUfgcccUfaCfacaags | | UCCUUGUGUAGGGCAAACCC | |
| 1646695 | csa | | gsa | | GCU | |
| AD- | gsgscca(Ada)CfgUfGfGfugaaggccs | | VPusAfsggcCfuUfCfaccaCfgUfuggccs | | CUGGCCAACGUGGUGAAGGC | |
| 1646696 | usa | | asg | | CUU | |
| AD- | gsgsagu(Ada)CfgGfCfUfccuucuucs | | VPusAfsgaaGfaAfGfgagcCfgUfacuccs | | UGGGAGUACGGCUCCUUCUU | |
| 1646697 | usa | | csa | | CUG | |
| AD- | gscscca(Gda)AfaGfCfAfggugaagas | | VPusUfsucuUfcAfCfcugcUfuCfugggc | | AGGCCCAGAAGCAGGUGAAG | |
| 1646698 | asa | | scsu | | AAG | |
| AD- | csasgaa(Gda)GfcGfCfUfcaagacgcs | | VPusAfsgcgUfcUfUfgagcGfcCfuucug | | AGCAGAAGGCGCUCAAGACG | |
| 1646699 | usa | | scsu | | CUG | |
| AD- | ususccg(Cda)UfaCfCfAfgagccugcs | | VPusAfsgcaGfgCfUfcuggUfaGfcggaa | | CCUUCCGCUACCAGAGCCUG | |
| 1646700 | usa | | sgsg | | CUG | |
| AD- | csusucc(Gda)CfaAfGfGfccuuccags | | VPusCfscugGfaAfGfgccuUfgCfggaag | | GACUUCCGCAAGGCCUUCCA | |
| 1646701 | gsa | | susc | | GGG | |
| AD- | gsasgug(Gda)AfaGfAfUfgggugggu | | VPusAfsaccCfaCfCfcaucUfuCfcacucs | | CGGAGUGGAAGAUGGGUGGG | |
| 1646702 | susa | | csg | | UUA | |
| AD- | gsgscca(Gda)CfaUfCfGfagacccugs | | VPusAfscagGfgUfCfucgaUfgCfuggcc | | ACGGCCAGCAUCGAGACCCU | |
| 1646703 | usa | | sgsu | | GUG | |
| AD- | csusgcu(Gda)GfcUfGfCfccuucuucs | | VPusGfsgaaGfaAfGfggcaGfcCfagcags | | CUCUGCUGGCUGCCCUUCUU | |
| 1646704 | csa | | asg | | CCU | |
| AD- | gsasccg(Cda)UfaCfCfUfcgccaucasc | | VPusGfsugaUfgGfCfgaggUfaGfcgguc | | UGGACCGCUACCUCGCCAUC | |
| 1646705 | sa | | scsa | | ACC | |
| AD- | csgsugg(Cda)CfaUfCfGfccaagacgs | | VPusGfscguCfuUfGfgcgaUfgGfccacg | | AUCGUGGCCAUCGCCAAGAC | |
| 1646706 | csa | | sasu | | GCC | |
| AD- | asusggg(Uda)CfuGfCfUfgauggcgcs | | VPusAfsgcgCfcAfUfcagcAfgAfcccaus | | GCAUGGGUCUGCUGAUGGCG | |
| 1646707 | usa | | gsc | | CUC | |
| AD- | gsgsgca(Uda)CfaUfCfAfugggcgucs | | VPusAfsgacGfcCfCfaugaUfgAfugcccs | | CUGGGCAUCAUCAUGGGCGU | |
| 1646708 | usa | | asg | | CUU | |
| AD- | gsasaca(Cda)AfcUfAfCfcagccagasg | | VPusCfsucuGfgCfUfgguaGfuGfuguuc | | AGGAACACACUACCAGCCAG | |
| 1646709 | sa | | scsu | | AGA | |
| AD- | asusugc(Cda)CfuGfGfAfccgcuaccs | | VPusAfsgguAfgCfGfguccAfgGfgcaau | | UCAUUGCCCUGGACCGCUAC | |
| 1646710 | usa | | sgsa | | CUC | |
| AD- | asuscuc(Gda)GfcCfCfUfgguguccus | | VPusAfsaggAfcAfCfcaggGfcCfgagau | | CCAUCUCGGCCCUGGUGUCC | |
| 1646711 | usa | | sgsg | | UUC | |
| AD- | asgscug(Cda)GfaGfCfGfccguuuccs | | VPusAfsggaAfaCfGfgcgcUfcGfcagcu | | ACAGCUGCGAGCGCCGUUUC | |
| 1646712 | usa | | sgsu | | CUC | |
| AD- | csasuca(Uda)CfuAfCfUfgccgcagcs | | VPusGfsgcuGfcGfGfcaguAfgAfugaug | | CCCAUCAUCUACUGCCGCAG | |
| 1646713 | csa | | sgsg | | CCC | |
| AD- | gscsaga(Cda)GfcUfCfAfccaaccucsu | | VPusAfsgagGfuUfGfgugaGfcGfucugc | | CUGCAGACGCUCACCAACCU | |
| 1646714 | sa | | sasg | | CUU | |
| AD- | csusucc(Ada)GfgGfAfCfugcucugcs | | VPusAfsgcaGfaGfCfagucCfcUfggaags | | GCCUUCCAGGGACUGCUCUG | |
| 1646715 | usa | | gsc | | CUG | |
| AD- | csusuca(Uda)CfaUfGfUfcccuggccs | | VPusUfsggcCfaGfGfgacaUfgAfugaag | | CUCUUCAUCAUGUCCCUGGC | |
| 1646716 | asa | | sasg | | CAG | |
| AD- | asasaca(Uda)GfcUfGfAfagucccggs | | VPusGfsccgGfgAfCfuucaGfcAfuguuu | | AGAAACAUGCUGAAGUCCCG | |
| 1646717 | csa | | scsu | | GCG | |
| AD- | asasggc(Cda)UfuCfCfAfccgcgagcs | | VPusAfsgcuCfgCfGfguggAfaGfgccuu | | UGAAGGCCUUCCACCGCGAG | |
| 1646718 | usa | | scsa | | CUG | |
| AD- | csasggc(Gda)CfaGfAfGfccucucucs | | VPusAfsgagAfgAfGfgcucUfgCfgccug | | ACCAGGCGCAGAGCCUCUCU | |
| 1646719 | usa | | sgsu | | CUG | |
| AD- | csgsagc(Cda)CfgGfUfAfaccugucgs | | VPusAfscgaCfaGfGfuuacCfgGfgcucg | | UCCGAGCCCGGUAACCUGUC | |
| 1646720 | usa | | sgsa | | GUC | |
| AD- | csusgug(Gda)AfcCfUfCfaguggacgs | | VPusAfscguCfcAfCfugagGfuCfcacags | | AGCUGUGGACCUCAGUGGAC | |
| 1646721 | usa | | csu | | GUG | |
| AD- | gscscac(Gda)GfaCfCfGfuugcacaasa | | VPusUfsuugUfgCfAfacggUfcCfguggc | | AAGCCACGGACCGUUGCACA | |
| 1646722 | sa | | susu | | AAA | |
| AD- | ascscuc(Gda)CfcCfUfUfccgcuaccsa | | VPusUfsgguAfgCfGfgaagGfgCfgaggu | | UCACCUCGCCCUUCCGCUAC | |
| 1646723 | sa | | sgsa | | CAG | |
| AD- | uscsaag(Ada)CfgCfUfGfggcaucaus | | VPusGfsaugAfuGfCfccagCfgUfcuuga | | GCUCAAGACGCUGGGCAUCA | |
| 1646724 | csa | | sgsc | | UCA | |
| AD- | gsgsaga(Gda)UfgGfCfUfugcugaugs | | VPusAfscauCfaGfCfaagcCfaCfucuccs | | UGGGAGAGUGGCUUGCUGAU | |
| 1646725 | usa | | csa | | GUU | |
| AD- | csusgcu(Cda)AfuCfGfUfggcgggcas | | VPusUfsugcCfcGfCfcacgAfuGfagcags | | UGCUGCUCAUCGUGGCGGGC | |
| 1646726 | asa | | csa | | AAU | |
| AD- | ascscag(Cda)CfaGfAfGfagaggagas | | VPusUfsucuCfcUfCfucucUfgGfcuggu | | CUACCAGCCAGAGAGAGGAG | |
| 1646727 | asa | | sasg | | AAU | |
| AD- | gscsugg(Uda)GfaUfCfGfuggccaucs | | VPusCfsgauGfgCfCfacgaUfcAfccagcs | | GUGCUGGUGAUCGUGGCCAU | |
| 1646728 | gsa | | asc | | CGC | |
| AD- | asasguc(Cda)CfgGfCfGfgcucuuccs | | VPusUfsggaAfgAfGfccgcCfgGfgacuu | | UGAAGUCCCGGCGGCUCUUC | |
| 1646729 | asa | | scsa | | CAG | |
| AD- | gsgsuca(Gda)GfcUfGfAfgggauuucs | | VPusAfsgaaAfuCfCfcucaGfcCfugaccs | | GUGGUCAGGCUGAGGGAUUU | |
| 1646730 | usa | | asc | | CUA | |
| AD- | uscsagu(Gda)GfaCfGfUfgcugugcgs | | VPusAfscgcAfcAfGfcacgUfcCfacugas | | CCUCAGUGGACGUGCUGUGC | |
| 1646731 | usa | | gsg | | GUG | |
| AD- | usasccu(Gda)CfgGfGfUfguuccgcgs | | VPusUfscgcGfgAfAfcaccCfgCfagguas | | UGUACCUGCGGGUGUUCCGC | |
| 1646732 | asa | | csa | | GAG | |
| AD- | ususucu(Gda)UfuUfGfUfgguccggcs | | VPusGfsgccGfgAfCfcacaAfaCfagaaas | | UUUUUCUGUUUGUGGUCCGG | |
| 1646733 | csa | | asa | | CCU | |
| AD- | ascscaa(Cda)CfgGfGfCfcuacgccasu | | VPusAfsuggCfgUfAfggccCfgGfuuggu | | UCACCAACCGGGCCUACGCC | |
| 1646734 | sa | | sgsa | | AUC | |

TABLE 4
Unmodified Sense and Antisense Strand Sequences of ADRB1 dsRNA Agents Comprising a GalNAc
Conjugate Targeting Ligand

| | | | | SEQ | |
|---|---|---|---|---|---|
| | | SEQ | | ID | Range in |
| Duplex Name | Sense Sequence 5′ to 3′ | ID NO: | Antisense Sequence 5′ to 3′ | NO: | NM_000684.3 |
| AD-1637961 | AAACAUGCUGAAGUCCCGGCU | | AGCCGGGACUUCAGCAUGUUUCU | | 3-23 |
| AD-1637971 | AAGUCCCGGCGGCUCUUCCAU | | AUGGAAGAGCCGCCGGGACUUCA | | 13-33 |
| AD-1638021 | CGAGCCCGGUAACCUGUCGUU | | AACGACAGGUUACCGGGCUCGGA | | 298-320 |
| AD-1638051 | CGCUGUCUCAGCAGUGGACAU | | AUGUCCACUGCUGAGACAGCGGC | | 420-442 |
| AD-1638077 | AUGGGUCUGCUGAUGGCGCUU | | AAGCGCCAUCAGCAGACCCAUGC | | 446-468 |
| AD-1638092 | GCGCUCAUCGUGCUGCUCAUU | | AAUGAGCAGCACGAUGAGCGCCA | | 461-483 |
| AD-1638104 | CUGCUCAUCGUGGCGGGCAAU | | AUUGCCCGCCACGAUGAGCAGCA | | 473-495 |
| AD-1638117 | CGGGCAAUGUGCUGGUGAUCU | | AGAUCACCAGCACAUUGCCCGCC | | 486-508 |
| AD-1638127 | GCUGGUGAUCGUGGCCAUCGU | | ACGAUGGCCACGAUCACCAGCAC | | 496-518 |
| AD-1638136 | CGUGGCCAUCGCCAAGACGCU | | AGCGUCUUGGCGAUGGCCACGAU | | 505-527 |
| AD-1638154 | GCAGACGCUCACCAACCUCUU | | AAGAGGUUGGUGAGCGUCUGCAG | | 532-554 |
| AD-1638163 | CACCAACCUCUUCAUCAUGUU | | AACAUGAUGAAGAGGUUGGUGAG | | 541-563 |
| AD-1638172 | CUUCAUCAUGUCCCUGGCCAU | | AUGGCCAGGGACAUGAUGAAGAG | | 550-572 |
| AD-1638196 | GGAGUACGGCUCCUUCUUCUU | | AAGAAGAAGGAGCCGUACUCCCA | | 637-659 |
| AD-1638205 | CUCCUUCUUCUGCGAGCUGUU | | AACAGCUCGCAGAAGAAGGAGCC | | 646-668 |
| AD-1638221 | CUGUGGACCUCAGUGGACGUU | | AACGUCCACUGAGGUCCACAGCU | | 662-684 |
| AD-1638230 | UCAGUGGACGUGCUGUGCGUU | | AACGCACAGCACGUCCACUGAGG | | 671-693 |
| AD-1638253 | GGCCAGCAUCGAGACCCUGUU | | AACAGGGUCUCGAUGCUGGCCGU | | 694-716 |
| AD-1638266 | ACCCUGUGUGUCAUUGCCCUU | | AAGGGCAAUGACACACAGGGUCU | | 707-729 |
| AD-1638278 | AUUGCCCUGGACCGCUACCUU | | AAGGUAGCGGUCCAGGGCAAUGA | | 719-741 |
| AD-1638287 | GACCGCUACCUCGCCAUCACU | | AGUGAUGGCGAGGUAGCGGUCCA | | 728-750 |
| AD-1638296 | CUCGCCAUCACCUCGCCCUUU | | AAAGGGCGAGGUGAUGGCGAGGU | | 737-759 |
| AD-1638305 | ACCUCGCCCUUCCGCUACCAU | | AUGGUAGCGGAAGGGCGAGGUGA | | 746-768 |
| AD-1638314 | UUCCGCUACCAGAGCCUGCUU | | AAGCAGGCUCUGGUAGCGGAAGG | | 755-777 |
| AD-1638347 | AUCUCGGCCCUGGUGUCCUUU | | AAAGGACACCAGGGCCGAGAUGG | | 818-840 |
| AD-1638356 | CUGGUGUCCUUCCUGCCCAUU | | AAUGGGCAGGAAGGACACCAGGG | | 827-849 |
| AD-1638370 | GCCCAUCCUCAUGCACUGGUU | | AACCAGUGCAUGAGGAUGGGCAG | | 841-863 |
| AD-1638393 | CUGCGACUUCGUCACCAACCU | | AGGUUGGUGACGAAGUCGCAGCA | | 910-932 |
| AD-1638406 | ACCAACCGGGCCUACGCCAUU | | AAUGGCGUAGGCCCGGUUGGUGA | | 923-945 |
| AD-1638422 | AUCGCCUCGUCCGUAGUCUCU | | AGAGACUACGGACGAGGCGAUGG | | 941-963 |
| AD-1638431 | UCCGUAGUCUCCUUCUACGUU | | AACGUAGAAGGAGACUACGGACG | | 950-972 |
| AD-1638435 | CUGUGCAUCAUGGCCUUCGUU | | AACGAAGGCCAUGAUGCACAGGG | | 974-996 |
| AD-1638444 | AUGGCCUUCGUGUACCUGCGU | | ACGCAGGUACACGAAGGCCAUGA | | 983-1005 |
| AD-1638456 | UACCUGCGGGUGUUCCGCGAU | | AUCGCGGAACACCCGCAGGUACA | | 995-1017 |
| AD-1638474 | GCCCAGAAGCAGGUGAAGAAU | | AUUCUUCACCUGCUUCUGGGCCU | | 1016-1038 |
| AD-1638485 | GGUGAAGAAGAUCGACAGCUU | | AAGCUGUCGAUCUUCUUCACCUG | | 1027-1049 |
| AD-1638500 | AGCUGCGAGCGCCGUUUCCUU | | AAGGAAACGGCGCUCGCAGCUGU | | 1043-1065 |
| AD-1638529 | CAGAAGGCGCUCAAGACGCUU | | AAGCGUCUUGAGCGCCUUCUGCU | | 1223-1245 |
| AD-1638539 | UCAAGACGCUGGGCAUCAUCU | | AGAUGAUGCCCAGCGUCUUGAGC | | 1233-1255 |
| AD-1638549 | GGGCAUCAUCAUGGGCGUCUU | | AAGACGCCCAUGAUGAUGCCCAG | | 1243-1265 |
| AD-1638561 | GGGCGUCUUCACGCUCUGCUU | | AAGCAGAGCGUGAAGACGCCCAU | | 1255-1277 |
| AD-1638576 | CUGCUGGCUGCCCUUCUUCCU | | AGGAAGAAGGGCAGCCAGCAGAG | | 1270-1292 |
| AD-1638585 | GCCCUUCUUCCUGGCCAACGU | | ACGUUGGCCAGGAAGAAGGGCAG | | 1279-1301 |
| AD-1638597 | GGCCAACGUGGUGAAGGCCUU | | AAGGCCUUCACCACGUUGGCCAG | | 1291-1313 |
| AD-1638610 | AAGGCCUUCCACCGCGAGCUU | | AAGCUCGCGGUGGAAGGCCUUCA | | 1304-1326 |
| AD-1638631 | CCUCUUCGUCUUCUUCAACUU | | AAGUUGAAGAAGACGAAGAGGCG | | 1336-1358 |
| AD-1638640 | CUUCUUCAACUGGCUGGGCUU | | AAGCCCAGCCAGUUGAAGAAGAC | | 1345-1367 |
| AD-1638659 | UACGCCAACUCGGCCUUCAAU | | AUUGAAGGCCGAGUUGGCGUAGC | | 1364-1386 |
| AD-1638662 | CAUCAUCUACUGCCGCAGCCU | | AGGCUGCGGCAGUAGAUGAUGGG | | 1387-1409 |
| AD-1638666 | CUUCCGCAAGGCCUUCCAGGU | | ACCUGGAAGGCCUUGCGGAAGUC | | 1411-1433 |
| AD-1638678 | CUUCCAGGGACUGCUCUGCUU | | AAGCAGAGCAGUCCCUGGAAGGC | | 1423-1445 |
| AD-1638705 | GACGACGACGAUGUCGUCGGU | | ACCGACGACAUCGUCGUCGUCGU | | 1556-1578 |
| AD-1638732 | CCUCGGAAUCCAAGGUGUAGU | | ACUACACCUUGGAUUCCGAGGCG | | 1680-1702 |
| AD-1638752 | GAGGAGAUCUGUGUUUACUUU | | AAAGUAAACACAGAUCUCCUCGU | | 1746-1768 |
| AD-1638762 | GUGUUUACUUAAGACCGAUAU | | AUAUCGGUCUUAAGUAAACACAG | | 1756-1778 |
| AD-1638771 | UAAGACCGAUAGCAGGUGAAU | | AUUCACCUGCUAUCGGUCUUAAG | | 1765-1787 |
| AD-1638780 | UAGCAGGUGAACUCGAAGCCU | | AGGCUUCGAGUUCACCUGCUAUC | | 1774-1796 |
| AD-1638794 | GAAGCCCACAAUCCUCGUCUU | | AAGACGAGGAUUGUGGGCUUCGA | | 1788-1810 |
| AD-1638803 | AAUCCUCGUCUGAAUCAUCCU | | AGGAUGAUUCAGACGAGGAUUGU | | 1797-1819 |
| AD-1638813 | UGAAUCAUCCGAGGCAAAGAU | | AUCUUUGCCUCGGAUGAUUCAGA | | 1807-1829 |
| AD-1638823 | GAGGCAAAGAGAAAAGCCACU | | AGUGGCUUUUCUCUUUGCCUCGG | | 1817-1839 |
| AD-1638838 | GCCACGGACCGUUGCACAAAU | | AUUUGUGCAACGGUCCGUGGCUU | | 1832-1854 |
| AD-1638859 | AGGAAAGUUUGGGAAGGGAUU | | AAUCCCUUCCCAAACUUUCCUUU | | 1853-1875 |
| AD-1638876 | GGAGAGUGGCUUGCUGAUGUU | | AACAUCAGCAAGCCACUCUCCCA | | 1874-1896 |
| AD-1638886 | UUGCUGAUGUUCCUUGUUGUU | | AACAACAAGGAACAUCAGCAAGC | | 1884-1906 |
| AD-1638890 | UUUCUGUUUGUGGUCCGGCCU | | AGGCCGGACCACAAACAGAAAAA | | 1953-1975 |
| AD-1638902 | GUCCGGCCUUCUUUUGUGUGU | | ACACACAAAAGAAGGCCGGACCA | | 1965-1987 |
| AD-1638911 | UCUUUUGUGUGUGCGUGUGAU | | AUCACACGCACACACAAAAGAAG | | 1974-1996 |
| AD-1638927 | GUGAUGCAUCUUUAGAUUUUU | | AAAAAUCUAAAGAUGCAUCACAC | | 1990-2012 |
| AD-1638933 | GGUGGUUUUUGACACUCUCUU | | AAGAGAGUGUCAAAAACCACCUG | | 2022-2044 |
| AD-1638943 | GACACUCUCUGAGAGGACCGU | | ACGGUCCUCUCAGAGAGUGUCAA | | 2032-2054 |
| AD-1638961 | GAGUGGAAGAUGGGUGGGUUU | | AAACCCACCCAUCUUCCACUCCG | | 2052-2074 |
| AD-1638968 | AAGGGAGAAGCAUUAGGAGGU | | ACCUCCUAAUGCUUCUCCCUUCC | | 2077-2099 |
| AD-1638971 | GAUUAAAAUCGAUCAUCGUGU | | ACACGAUGAUCGAUUUUAAUCCC | | 2098-2120 |
| AD-1638985 | AUCGUGGCUCCCAUCCCUUUU | | AAAAGGGAUGGGAGCCACGAUGA | | 2112-2134 |
| AD-1638994 | CCCAUCCCUUUCCCGGGAACU | | AGUUCCCGGGAAAGGGAUGGGAG | | 2121-2143 |
| AD-1639007 | CGGGAACAGGAACACACUACU | | AGUAGUGUGUUCCUGUUCCCGGG | | 2134-2156 |
| AD-1639016 | GAACACACUACCAGCCAGAGU | | ACUCUGGCUGGUAGUGUGUUCCU | | 2143-2165 |
| AD-1639025 | ACCAGCCAGAGAGAGGAGAAU | | AUUCUCCUCUCUCUGGCUGGUAG | | 2152-2174 |
| AD-1639034 | AGAGAGGAGAAUGACAGUUUU | | AAAACUGUCAUUCUCCUCUCUCU | | 2161-2183 |
| AD-1639044 | AUGACAGUUUGUCAAGACAUU | | AAUGUCUUGACAAACUGUCAUUC | | 2171-2193 |
| AD-1639057 | AAGACAUAUUUCCUUUUGCUU | | AAGCAAAAGGAAAUAUGUCUUGA | | 2184-2206 |
| AD-1639068 | CCUUUUGCUUUCCAGAGAAAU | | AUUUCUCUGGAAAGCAAAAGGAA | | 2195-2217 |
| AD-1639077 | UUCCAGAGAAAUUUCAUUUUU | | AAAAAUGAAAUUUCUCUGGAAAG | | 2204-2226 |
| AD-1639087 | AGUAAUGAUUUCUGCUGUUAU | | AUAACAGCAGAAAUCAUUACUUA | | 2232-2254 |
| AD-1639099 | UGCUGUUAUGAAAGCAAAGAU | | AUCUUUGCUUUCAUAACAGCAGA | | 2244-2266 |
| AD-1639108 | AAAGCAAAGAGAAAGGAUGGU | | ACCAUCCUUUCUCUUUGCUUUCA | | 2254-2276 |
| AD-1639116 | AGAAAGGAUGGAGGCAAAAUU | | AAUUUUGCCUCCAUCCUUUCUCU | | 2263-2285 |
| AD-1639123 | AUCACGUUUCAAGAAAUGUUU | | AAACAUUUCUUGAAACGUGAUUU | | 2291-2313 |
| AD-1639133 | AAGAAAUGUUAAGCUCUUCUU | | AAGAAGAGCUUAACAUUUCUUGA | | 2301-2323 |
| AD-1639143 | AAGCUCUUCUUGGAACAAGCU | | AGCUUGUUCCAAGAAGAGCUUAA | | 2311-2333 |
| AD-1639145 | CACCUUGCUUUCCUUGUGUAU | | AUACACAAGGAAAGCAAGGUGGG | | 2333-2355 |
| AD-1639157 | CUUGUGUAGGGCAAACCCGCU | | AGCGGGUUUGCCCUACACAAGGA | | 2345-2367 |
| AD-1639174 | GGUCAGGCUGAGGGAUUUCUU | | AAGAAAUCCCUCAGCCUGACCAC | | 2385-2407 |
| AD-1639183 | GAGGGAUUUCUACCUCACACU | | AGUGUGAGGUAGAAAUCCCUCAG | | 2394-2416 |
| AD-1639192 | CUACCUCACACUGUGCAUUUU | | AAAAUGCACAGUGUGAGGUAGAA | | 2403-2425 |
| AD-1639206 | GCAUUUGCACAGCAGAUAGAU | | AUCUAUCUGCUGUGCAAAUGCAC | | 2417-2439 |
| AD-1639220 | GAUAGAAAGACUUGUUUAUAU | | AUAUAAACAAGUCUUUCUAUCUG | | 2431-2453 |
| AD-1639226 | CUUGUUUAUAUUAAACAGCUU | | AAGCUGUUUAAUAUAAACAAGUC | | 2441-2463 |
| AD-1639240 | GUAUCAAUAUUAGUUGGAAGU | | ACUUCCAACUAAUAUUGAUACAU | | 2468-2490 |
| AD-1639263 | CAGGCGCAGAGCCUCUCUCUU | | AAGAGAGAGGCUCUGCGCCUGGU | | 2491-2513 |
| AD-1639272 | AGCCUCUCUCUGUGACAUGUU | | AACAUGUCACAGAGAGAGGCUCU | | 2500-2522 |
| AD-1639282 | UGUGACAUGUGACUCUGUCAU | | AUGACAGAGUCACAUGUCACAGA | | 2510-2532 |
| AD-1639295 | UCUGUCAAUUGAAGACAGGAU | | AUCCUGUCUUCAAUUGACAGAGU | | 2523-2545 |
| AD-1639304 | UGAAGACAGGACAUUAAAAGU | | ACUUUUAAUGUCCUGUCUUCAAU | | 2532-2554 |
| AD-1639313 | GACAUUAAAAGAGAGCGAGAU | | AUCUCGCUCUCUUUUAAUGUCCU | | 2541-2563 |
| AD-1639321 | AGCGAGAGAGAGAAACAGUUU | | AAACUGUUUCUCUCUCUCGCUCU | | 2554-2576 |
| AD-1639332 | GAAACAGUUCAGAUUACUGCU | | AGCAGUAAUCUGAACUGUUUCUC | | 2565-2587 |
| AD-1639342 | AGAUUACUGCACAUGUGGAUU | | AAUCCACAUGUGCAGUAAUCUGA | | 2575-2597 |
| AD-1639362 | GUGGUUCAAAAUGCCAUUUUU | | AAAAAUGGCAUUUUGAACCACUC | | 2618-2640 |
| AD-1639373 | UGCCAUUUUUGCACAGUGUUU | | AAACACUGUGCAAAAAUGGCAUU | | 2629-2651 |
| AD-1639387 | AGUGUUAGGAAUUACAAAAUU | | AAUUUUGUAAUUCCUAACACUGU | | 2643-2665 |
| AD-1639396 | AAUUACAAAAUCCACAGAAGU | | ACUUCUGUGGAUUUUGUAAUUCC | | 2652-2674 |
| AD-1639407 | CCACAGAAGAUGUUACUUGCU | | AGCAAGUAACAUCUUCUGUGGAU | | 2663-2685 |
| AD-1639429 | GGGCAGAUCUUAAAUAAAAUU | | AAUUUUAUUUAAGAUCUGCCCAG | | 2722-2744 |
| AD-1639437 | AUUCAAACUCUACUUCUGUUU | | AAACAGAAGUAGAGUUUGAAUUU | | 2740-2762 |
| AD-1639446 | CUACUUCUGUUGUCUAGUAUU | | AAUACUAGACAACAGAAGUAGAG | | 2749-2771 |
| AD-1639460 | UAGUAUGUUAUUGAGCUAAUU | | AAUUAGCUCAAUAACAUACUAGA | | 2763-2785 |
| AD-1639469 | AUUGAGCUAAUGAUUCAUUGU | | ACAAUGAAUCAUUAGCUCAAUAA | | 2772-2794 |
| AD-1639485 | AUUGGGAAAAUACCUUUUUAU | | AUAAAAAGGUAUUUUCCCAAUGA | | 2788-2810 |
| AD-1639495 | CUUUUUAUACUCCUUUAUCAU | | AUGAUAAAGGAGUAUAAAAAGGU | | 2801-2823 |
| AD-1639505 | UCCUUUAUCAUGGUACUGUAU | | AUACAGUACCAUGAUAAAGGAGU | | 2811-2833 |
| AD-1639521 | UGUAACUGUAUCCAUAUUAUU | | AAUAAUAUGGAUACAGUUACAGU | | 2827-2849 |
| AD-1639526 | UUAUGUCCAAGUGCCCACGUU | | AACGUGGGCACUUGGACAUAAAA | | 2881-2903 |
| AD-1639541 | CACGUGAAUUUGCUGGUGAAU | | AUUCACCAGCAAAUUCACGUGGG | | 2896-2918 |
| AD-1639550 | UUGCUGGUGAAAGUUAGCACU | | AGUGCUAACUUUCACCAGCAAAU | | 2905-2927 |
| AD-1639559 | AAAGUUAGCACUUGUGUGUAU | | AUACACACAAGUGCUAACUUUCA | | 2914-2936 |
| AD-1639568 | ACUUGUGUGUAAAUUCUACUU | | AAGUAGAAUUUACACACAAGUGC | | 2923-2945 |
| AD-1639577 | UAAAUUCUACUUCCUCUUGUU | | AACAAGAGGAAGUAGAAUUUACA | | 2932-2954 |
| AD-1639591 | CUUGUGUGUUUUACCAAGUAU | | AUACUUGGUAAAACACACAAGAG | | 2947-2969 |
| AD-1639599 | UUUACCAAGUAUUUAUACUCU | | AGAGUAUAAAUACUUGGUAAAAC | | 2956-2978 |
| AD-1639615 | ACUCUGGUGCAACUAACUACU | | AGUAGUUAGUUGCACCAGAGUAU | | 2972-2994 |
| AD-1639628 | UAACUACUGUGUGAGGAAUUU | | AAAUUCCUCACACAGUAGUUAGU | | 2985-3007 |
| AD-1639642 | GGAAUUGGUCCAUGUGCAAUU | | AAUUGCACAUGGACCAAUUCCUC | | 2999-3021 |
| AD-1639655 | GUGCAAUAAAUACCAAUGAAU | | AUUCAUUGGUAUUUAUUGCACAU | | 3012-3034 |

TABLE 5
Modified Sense and Antisense Strand Sequences of ADRB1 dsRNA Agents Comprising a GalNAc
Conjugate Targeting Ligand

| | | SEQ | | SEQ | | SEQ |
|---|---|---|---|---|---|---|
| Duplex | | ID | | ID | mRNA Target Sequence | ID |
| Name | oligoSeq | NO: | Antisense Sequence 5′ to 3′ | NO: | 5′ to 3′ | NO: |
| AD-1637961 | asasacauGfcUfGfAfagucccggc | | asGfsccgGfgAfCfuucaGfcAfuguu | | AGAAACAUGCUGAAGUCCCGG | |
| | uL96 | | uscsu | | CG | |
| AD-1637971 | asasguccCfgGfCfGfgcucuucca | | asUfsggaAfgAfGfccgcCfgGfgacu | | UGAAGUCCCGGCGGCUCUUCC | |
| | uL96 | | uscsa | | AG | |
| AD-1638021 | csgsagccCfgGfUfAfaccugucgu | | asAfscgaCfaGfGfuuacCfgGfgcucg | | UCCGAGCCCGGUAACCUGUCG | |
| | uL96 | | sgsa | | UC | |
| AD-1638051 | csgscuguCfuCfAfGfcaguggaca | | asUfsgucCfaCfUfgcugAfgAfcagc | | GCCGCUGUCUCAGCAGUGGAC | |
| | uL96 | | gsgsc | | AG | |
| AD-1638077 | asusggguCfuGfCfUfgauggcgcu | | asAfsgcgCfcAfUfcagcAfgAfcccau | | GCAUGGGUCUGCUGAUGGCGC | |
| | uL96 | | sgsc | | UC | |
| AD-1638092 | gscsgcucAfuCfGfUfgcugcucau | | asAfsugaGfcAfGfcacgAfuGfagcg | | UGGCGCUCAUCGUGCUGCUCA | |
| | uL96 | | cscsa | | UC | |
| AD-1638104 | csusgcucAfuCfGfUfggcgggcaa | | asUfsugcCfcGfCfcacgAfuGfagcag | | UGCUGCUCAUCGUGGCGGGCA | |
| | uL96 | | scsa | | AU | |
| AD-1638117 | csgsggcaAfuGfUfGfcuggugauc | | asGfsaucAfcCfAfgcacAfuUfgcccg | | GGCGGGCAAUGUGCUGGUGA | |
| | uL96 | | SCSC | | UCG | |
| AD-1638127 | gscsugguGfaUfCfGfuggccaucg | | asCfsgauGfgCfCfacgaUfcAfccagc | | GUGCUGGUGAUCGUGGCCAUC | |
| | uL96 | | sasc | | GC | |
| AD-1638136 | csgsuggcCfaUfCfGfccaagacgcu | | asGfscguCfuUfGfgcgaUfgGfccac | | AUCGUGGCCAUCGCCAAGACG | |
| | L96 | | gsasu | | CC | |
| AD-1638154 | gscsagacGfcUfCfAfccaaccucuu | | asAfsgagGfuUfGfgugaGfcGfucug | | CUGCAGACGCUCACCAACCUC | |
| | L96 | | csasg | | UU | |
| AD-1638163 | csasccaaCfcUfCfUfucaucauguu | | asAfscauGfaUfGfaagaGfgUfuggu | | CUCACCAACCUCUUCAUCAUG | |
| | L96 | | gsasg | | UC | |
| AD-1638172 | csusucauCfaUfGfUfcccuggcca | | asUfsggcCfaGfGfgacaUfgAfugaa | | CUCUUCAUCAUGUCCCUGGCC | |
| | uL96 | | gsasg | | AG | |
| AD-1638196 | gsgsaguaCfgGfCfUfccuucuucu | | asAfsgaaGfaAfGfgagcCfgUfacucc | | UGGGAGUACGGCUCCUUCUUC | |
| | uL96 | | scsa | | UG | |
| AD-1638205 | csusccuuCfuUfCfUfgcgagcugu | | asAfscagCfuCfGfcagaAfgAfaggag | | GGCUCCUUCUUCUGCGAGCUG | |
| | uL96 | | SCSC | | UG | |
| AD-1638221 | csusguggAfcCfUfCfaguggacgu | | asAfscguCfcAfCfugagGfuCfcacag | | AGCUGUGGACCUCAGUGGACG | |
| | uL96 | | scsu | | UG | |
| AD-1638230 | uscsagugGfaCfGfUfgcugugcgu | | asAfscgcAfcAfGfcacgUfcCfacuga | | CCUCAGUGGACGUGCUGUGCG | |
| | uL96 | | sgsg | | UG | |
| AD-1638253 | gsgsccagCfaUfCfGfagacccugu | | asAfscagGfgUfCfucgaUfgCfuggc | | ACGGCCAGCAUCGAGACCCUG | |
| | uL96 | | csgsu | | UG | |
| AD-1638266 | ascsccugUfgUfGfUfcauugcccu | | asAfsgggCfaAfUfgacaCfaCfagggu | | AGACCCUGUGUGUCAUUGCCC | |
| | uL96 | | scsu | | UG | |
| AD-1638278 | asusugccCfuGfGfAfccgcuaccu | | asAfsgguAfgCfGfguccAfgGfgcaa | | UCAUUGCCCUGGACCGCUACC | |
| | uL96 | | usgsa | | UC | |
| AD-1638287 | gsasccgcUfaCfCfUfcgccaucacu | | asGfsugaUfgGfCfgaggUfaGfcggu | | UGGACCGCUACCUCGCCAUCA | |
| | L96 | | cscsa | | CC | |
| AD-1638296 | csuscgccAfuCfAfCfcucgcccuu | | asAfsaggGfcGfAfggugAfuGfgcga | | ACCUCGCCAUCACCUCGCCCU | |
| | uL96 | | gsgsu | | UC | |
| AD-1638305 | ascscucgCfcCfUfUfccgcuaccau | | asUfsgguAfgCfGfgaagGfgCfgagg | | UCACCUCGCCCUUCCGCUACC | |
| | L96 | | usgsa | | AG | |
| AD-1638314 | ususccgcUfaCfCfAfgagccugcu | | asAfsgcaGfgCfUfcuggUfaGfcgga | | CCUUCCGCUACCAGAGCCUGC | |
| | uL96 | | asgsg | | UG | |
| AD-1638347 | asuscucgGfcCfCfUfgguguccuu | | asAfsaggAfcAfCfcaggGfcCfgagau | | CCAUCUCGGCCCUGGUGUCCU | |
| | uL96 | | sgsg | | UC | |
| AD-1638356 | csusggugUfcCfUfUfccugcccau | | asAfsuggGfcAfGfgaagGfaCfaccag | | CCCUGGUGUCCUUCCUGCCCA | |
| | uL96 | | sgsg | | UC | |
| AD-1638370 | gscsccauCfcUfCfAfugcacuggu | | asAfsccaGfuGfCfaugaGfgAfuggg | | CUGCCCAUCCUCAUGCACUGG | |
| | uL96 | | csasg | | UG | |
| AD-1638393 | csusgcgaCfuUfCfGfucaccaaccu | | asGfsguuGfgUfGfacgaAfgUfcgca | | UGCUGCGACUUCGUCACCAAC | |
| | L96 | | gscsa | | CG | |
| AD-1638406 | ascscaacCfgGfGfCfcuacgccauu | | asAfsuggCfgUfAfggccCfgGfuugg | | UCACCAACCGGGCCUACGCCA | |
| | L96 | | usgsa | | UC | |
| AD-1638422 | asuscgccUfcGfUfCfcguagucuc | | asGfsagaCfuAfCfggacGfaGfgcgau | | CCAUCGCCUCGUCCGUAGUCU | |
| | uL96 | | sgsg | | CC | |
| AD-1638431 | uscscguaGfuCfUfCfcuucuacgu | | asAfscguAfgAfAfggagAfcUfacgg | | CGUCCGUAGUCUCCUUCUACG | |
| | uL96 | | ascsg | | UG | |
| AD-1638435 | csusgugcAfuCfAfUfggccuucgu | | asAfscgaAfgGfCfcaugAfuGfcacag | | CCCUGUGCAUCAUGGCCUUCG | |
| | uL96 | | sgsg | | UG | |
| AD-1638444 | asusggccUfuCfGfUfguaccugcg | | asCfsgcaGfgUfAfcacgAfaGfgccau | | UCAUGGCCUUCGUGUACCUGC | |
| | uL96 | | sgsa | | GG | |
| AD-1638456 | usasccugCfgGfGfUfguuccgcga | | asUfscgcGfgAfAfcaccCfgCfaggua | | UGUACCUGCGGGUGUUCCGCG | |
| | uL96 | | scsa | | AG | |
| AD-1638474 | gscsccagAfaGfCfAfggugaagaa | | asUfsucuUfcAfCfcugcUfuCfuggg | | AGGCCCAGAAGCAGGUGAAG | |
| | uL96 | | cscsu | | AAG | |
| AD-1638485 | gsgsugaaGfaAfGfAfucgacagcu | | asAfsgcuGfuCfGfaucuUfcUfucacc | | CAGGUGAAGAAGAUCGACAG | |
| | uL96 | | susg | | CUG | |
| AD-1638500 | asgscugcGfaGfCfGfccguuuccu | | asAfsggaAfaCfGfgcgcUfcGfcagcu | | ACAGCUGCGAGCGCCGUUUCC | |
| | uL96 | | sgsu | | UC | |
| AD-1638529 | csasgaagGfcGfCfUfcaagacgcuu | | asAfsgcgUfcUfUfgagcGfcCfuucu | | AGCAGAAGGCGCUCAAGACGC | |
| | L96 | | gscsu | | UG | |
| AD-1638539 | uscsaagaCfgCfUfGfggcaucauc | | asGfsaugAfuGfCfccagCfgUfcuug | | GCUCAAGACGCUGGGCAUCAU | |
| | uL96 | | asgsc | | CA | |
| AD-1638549 | gsgsgcauCfaUfCfAfugggcgucu | | asAfsgacGfcCfCfaugaUfgAfugccc | | CUGGGCAUCAUCAUGGGCGUC | |
| | uL96 | | sasg | | UU | |
| AD-1638561 | gsgsgcguCfuUfCfAfcgcucugcu | | asAfsgcaGfaGfCfgugaAfgAfcgccc | | AUGGGCGUCUUCACGCUCUGC | |
| | uL96 | | sasu | | UG | |
| AD-1638576 | csusgcugGfcUfGfCfccuucuucc | | asGfsgaaGfaAfGfggcaGfcCfagcag | | CUCUGCUGGCUGCCCUUCUUC | |
| | uL96 | | sasg | | CU | |
| AD-1638585 | gscsccuuCfuUfCfCfuggccaacg | | asCfsguuGfgCfCfaggaAfgAfaggg | | CUGCCCUUCUUCCUGGCCAAC | |
| | uL96 | | csasg | | GU | |
| AD-1638597 | gsgsccaaCfgUfGfGfugaaggccu | | asAfsggcCfuUfCfaccaCfgUfuggcc | | CUGGCCAACGUGGUGAAGGCC | |
| | uL96 | | sasg | | UU | |
| AD-1638610 | asasggccUfuCfCfAfccgcgagcu | | asAfsgcuCfgCfGfguggAfaGfgccu | | UGAAGGCCUUCCACCGCGAGC | |
| | uL96 | | uscsa | | UG | |
| AD-1638631 | cscsucuuCfgUfCfUfucuucaacu | | asAfsguuGfaAfGfaagaCfgAfagag | | CGCCUCUUCGUCUUCUUCAAC | |
| | uL96 | | gscsg | | UG | |
| AD-1638640 | csusucuuCfaAfCfUfggcugggcu | | asAfsgccCfaGfCfcaguUfgAfagaag | | GUCUUCUUCAACUGGCUGGGC | |
| | uL96 | | sasc | | UA | |
| AD-1638659 | usascgccAfaCfUfCfggccuucaau | | asUfsugaAfgGfCfcgagUfuGfgcgu | | GCUACGCCAACUCGGCCUUCA | |
| | L96 | | asgsc | | AC | |
| AD-1638662 | csasucauCfuAfCfUfgccgcagccu | | asGfsgcuGfcGfGfcaguAfgAfugau | | CCCAUCAUCUACUGCCGCAGC | |
| | L96 | | gsgsg | | CC | |
| AD-1638666 | csusuccgCfaAfGfGfccuuccagg | | asCfscugGfaAfGfgccuUfgCfggaa | | GACUUCCGCAAGGCCUUCCAG | |
| | uL96 | | gsusc | | GG | |
| AD-1638678 | csusuccaGfgGfAfCfugcucugcu | | asAfsgcaGfaGfCfagucCfcUfggaag | | GCCUUCCAGGGACUGCUCUGC | |
| | uL96 | | sgsc | | UG | |
| AD-1638705 | gsascgacGfaCfGfAfugucgucgg | | asCfscgaCfgAfCfaucgUfcGfucguc | | ACGACGACGACGAUGUCGUCG | |
| | uL96 | | sgsu | | GG | |
| AD-1638732 | cscsucggAfaUfCfCfaagguguag | | asCfsuacAfcCfUfuggaUfuCfcgagg | | CGCCUCGGAAUCCAAGGUGUA | |
| | uL96 | | scsg | | GG | |
| AD-1638752 | gsasggagAfuCfUfGfuguuuacuu | | asAfsaguAfaAfCfacagAfuCfuccuc | | ACGAGGAGAUCUGUGUUUAC | |
| | uL96 | | sgsu | | UUA | |
| AD-1638762 | gsusguuuAfcUfUfAfagaccgaua | | asUfsaucGfgUfCfuuaaGfuAfaacac | | CUGUGUUUACUUAAGACCGA | |
| | uL96 | | sasg | | UAG | |
| AD-1638771 | usasagacCfgAfUfAfgcaggugaa | | asUfsucaCfcUfGfcuauCfgGfucuua | | CUUAAGACCGAUAGCAGGUG | |
| | uL96 | | sasg | | AAC | |
| AD-1638780 | usasgcagGfuGfAfAfcucgaagcc | | asGfsgcuUfcGfAfguucAfcCfugcu | | GAUAGCAGGUGAACUCGAAG | |
| | uL96 | | asusc | | CCC | |
| AD-1638794 | gsasagccCfaCfAfAfuccucgucu | | asAfsgacGfaGfGfauugUfgGfgcuu | | UCGAAGCCCACAAUCCUCGUC | |
| | uL96 | | csgsa | | UG | |
| AD-1638803 | asasuccuCfgUfCfUfgaaucauccu | | asGfsgauGfaUfUfcagaCfgAfggau | | ACAAUCCUCGUCUGAAUCAUC | |
| | L96 | | usgsu | | CG | |
| AD-1638813 | usgsaaucAfuCfCfGfaggcaaaga | | asUfscuuUfgCfCfucggAfuGfauuc | | UCUGAAUCAUCCGAGGCAAAG | |
| | uL96 | | asgsa | | AG | |
| AD-1638823 | gsasggcaAfaGfAfGfaaaagccacu | | asGfsuggCfuUfUfucucUfuUfgccu | | CCGAGGCAAAGAGAAAAGCCA | |
| | L96 | | csgsg | | CG | |
| AD-1638838 | gscscacgGfaCfCfGfuugcacaaau | | asUfsuugUfgCfAfacggUfcCfgugg | | AAGCCACGGACCGUUGCACAA | |
| | L96 | | csusu | | AA | |
| AD-1638859 | asgsgaaaGfuUfUfGfggaagggau | | asAfsuccCfuUfCfccaaAfcUfuuccu | | AAAGGAAAGUUUGGGAAGGG | |
| | uL96 | | susu | | AUG | |
| AD-1638876 | gsgsagagUfgGfCfUfugcugaugu | | asAfscauCfaGfCfaagcCfaCfucucc | | UGGGAGAGUGGCUUGCUGAU | |
| | uL96 | | scsa | | GUU | |
| AD-1638886 | ususgcugAfuGfUfUfccuuguugu | | asAfscaaCfaAfGfgaacAfuCfagcaa | | GCUUGCUGAUGUUCCUUGUU | |
| | uL96 | | sgsc | | GUU | |
| AD-1638890 | ususucugUfuUfGfUfgguccggcc | | asGfsgccGfgAfCfcacaAfaCfagaaa | | UUUUUCUGUUUGUGGUCCGG | |
| | uL96 | | sasa | | CCU | |
| AD-1638902 | gsusccggCfcUfUfCfuuuugugug | | asCfsacaCfaAfAfagaaGfgCfcggac | | UGGUCCGGCCUUCUUUUGUGU | |
| | uL96 | | scsa | | GU | |
| AD-1638911 | uscsuuuuGfuGfUfGfugcguguga | | asUfscacAfcGfCfacacAfcAfaaaga | | CUUCUUUUGUGUGUGCGUGU | |
| | uL96 | | sasg | | GAU | |
| AD-1638927 | gsusgaugCfaUfCfUfuuagauuuu | | asAfsaaaUfcUfAfaagaUfgCfaucac | | GUGUGAUGCAUCUUUAGAUU | |
| | uL96 | | sasc | | UUU | |
| AD-1638933 | gsgsugguUfuUfUfGfacacucucu | | asAfsgagAfgUfGfucaaAfaAfccacc | | CAGGUGGUUUUUGACACUCUC | |
| | uL96 | | susg | | UG | |
| AD-1638943 | gsascacuCfuCfUfGfagaggaccg | | asCfsgguCfcUfCfucagAfgAfgugu | | UUGACACUCUCUGAGAGGACC | |
| | uL96 | | csasa | | GG | |
| AD-1638961 | gsasguggAfaGfAfUfgggugggu | | asAfsaccCfaCfCfcaucUfuCfcacuc | | CGGAGUGGAAGAUGGGUGGG | |
| | uuL96 | | scsg | | UUA | |
| AD-1638968 | asasgggaGfaAfGfCfauuaggagg | | asCfscucCfuAfAfugcuUfcUfcccuu | | GGAAGGGAGAAGCAUUAGGA | |
| | uL96 | | ScSC | | GGG | |
| AD-1638971 | gsasuuaaAfaUfCfGfaucaucgug | | asCfsacgAfuGfAfucgaUfuUfuaau | | GGGAUUAAAAUCGAUCAUCG | |
| | uL96 | | CSCSC | | UGG | |
| AD-1638985 | asuscgugGfcUfCfCfcaucccuuu | | asAfsaagGfgAfUfgggaGfcCfacga | | UCAUCGUGGCUCCCAUCCCUU | |
| | uL96 | | usgsa | | UC | |
| AD-1638994 | cscscaucCfcUfUfUfcccgggaacu | | asGfsuucCfcGfGfgaaaGfgGfaugg | | CUCCCAUCCCUUUCCCGGGAA | |
| | L96 | | gsasg | | CA | |
| AD-1639007 | csgsggaaCfaGfGfAfacacacuacu | | asGfsuagUfgUfGfuuccUfgUfuccc | | CCCGGGAACAGGAACACACUA | |
| | L96 | | gsgsg | | CC | |
| AD-1639016 | gsasacacAfcUfAfCfcagccagagu | | asCfsucuGfgCfUfgguaGfuGfuguu | | AGGAACACACUACCAGCCAGA | |
| | L96 | | cscsu | | GA | |
| AD-1639025 | ascscagcCfaGfAfGfagaggagaau | | asUfsucuCfcUfCfucucUfgGfcugg | | CUACCAGCCAGAGAGAGGAGA | |
| | L96 | | usasg | | AU | |
| AD-1639034 | asgsagagGfaGfAfAfugacaguuu | | asAfsaacUfgUfCfauucUfcCfucucu | | AGAGAGAGGAGAAUGACAGU | |
| | uL96 | | scsu | | UUG | |
| AD-1639044 | asusgacaGfuUfUfGfucaagacau | | asAfsuguCfuUfGfacaaAfcUfguca | | GAAUGACAGUUUGUCAAGAC | |
| | uL96 | | ususc | | AUA | |
| AD-1639057 | asasgacaUfaUfUfUfccuuuugcu | | asAfsgcaAfaAfGfgaaaUfaUfgucu | | UCAAGACAUAUUUCCUUUUGC | |
| | uL96 | | usgsa | | UU | |
| AD-1639068 | cscsuuuuGfcUfUfUfccagagaaa | | asUfsuucUfcUfGfgaaaGfcAfaaagg | | UUCCUUUUGCUUUCCAGAGAA | |
| | uL96 | | sasa | | AU | |
| AD-1639077 | ususccagAfgAfAfAfuuucauuuu | | asAfsaaaUfgAfAfauuuCfuCfugga | | CUUUCCAGAGAAAUUUCAUU | |
| | uL96 | | asasg | | UUA | |
| AD-1639087 | asgsuaauGfaUfUfUfcugcuguua | | asUfsaacAfgCfAfgaaaUfcAfuuacu | | UAAGUAAUGAUUUCUGCUGU | |
| | uL96 | | susa | | UAU | |
| AD-1639099 | usgscuguUfaUfGfAfaagcaaaga | | asUfscuuUfgCfUfuucaUfaAfcagca | | UCUGCUGUUAUGAAAGCAAA | |
| | uL96 | | sgsa | | GAG | |
| AD-1639108 | asasagcaAfaGfAfGfaaaggaugg | | asCfscauCfcUfUfucucUfuUfgcuu | | UGAAAGCAAAGAGAAAGGAU | |
| | uL96 | | uscsa | | GGA | |
| AD-1639116 | asgsaaag GfaUfGfGfaggcaaaau | | asAfsuuuUfgCfCfuccaUfcCfuuuc | | AGAGAAAGGAUGGAGGCAAA | |
| | uL96 | | uscsu | | AUA | |
| AD-1639123 | asuscacgUfuUfCfAfagaaauguu | | asAfsacaUfuUfCfuugaAfaCfguga | | AAAUCACGUUUCAAGAAAUG | |
| | uL96 | | ususu | | UUA | |
| AD-1639133 | asasgaaaUfgUfUfAfagcucuucu | | asAfsgaaGfaGfCfuuaaCfaUfuucuu | | UCAAGAAAUGUUAAGCUCUU | |
| | uL96 | | sgsa | | CUU | |
| AD-1639143 | asasgcucUfuCfUfUfggaacaagc | | asGfscuuGfuUfCfcaagAfaGfagcu | | UUAAGCUCUUCUUGGAACAA | |
| | uL96 | | usasa | | GCC | |
| AD-1639145 | csasccuuGfcUfUfUfccuugugua | | asUfsacaCfaAfGfgaaaGfcAfaggug | | CCCACCUUGCUUUCCUUGUGU | |
| | uL96 | | sgsg | | AG | |
| AD-1639157 | csusugugUfaGfGfGfcaaacccgc | | asGfscggGfuUfUfgcccUfaCfacaag | | UCCUUGUGUAGGGCAAACCCG | |
| | uL96 | | sgsa | | CU | |
| AD-1639174 | gsgsucagGfcUfGfAfgggauuucu | | asAfsgaaAfuCfCfcucaGfcCfugacc | | GUGGUCAGGCUGAGGGAUUU | |
| | uL96 | | sasc | | CUA | |
| AD-1639183 | gsasgggaUfuUfCfUfaccucacac | | asGfsuguGfaGfGfuagaAfaUfcccu | | CUGAGGGAUUUCUACCUCACA | |
| | uL96 | | csasg | | CU | |
| AD-1639192 | csusaccuCfaCfAfCfugugcauuu | | asAfsaauGfcAfCfagugUfgAfggua | | UUCUACCUCACACUGUGCAUU | |
| | uL96 | | gsasa | | UG | |
| AD-1639206 | gscsauuuGfcAfCfAfgcagauaga | | asUfscuaUfcUfGfcuguGfcAfaaug | | GUGCAUUUGCACAGCAGAUA | |
| | uL96 | | csasc | | GAA | |
| AD-1639220 | gsasuagaAfaGfAfCfuuguuuaua | | asUfsauaAfaCfAfagucUfuUfcuauc | | CAGAUAGAAAGACUUGUUUA | |
| | uL96 | | susg | | UAU | |
| AD-1639226 | csusuguuUfaUfAfUfuaaacagcu | | asAfsgcuGfuUfUfaauaUfaAfacaag | | GACUUGUUUAUAUUAAACAG | |
| | uL96 | | susc | | CUU | |
| AD-1639240 | gsusaucaAfuAfUfUfaguuggaag | | asCfsuucCfaAfCfuaauAfuUfgauac | | AUGUAUCAAUAUUAGUUGGA | |
| | uL96 | | sasu | | AGG | |
| AD-1639263 | csasggcgCfaGfAfGfccucucucu | | asAfsgagAfgAfGfgcucUfgCfgccu | | ACCAGGCGCAGAGCCUCUCUC | |
| | uL96 | | gsgsu | | UG | |
| AD-1639272 | asgsccucUfcUfCfUfgugacaugu | | asAfscauGfuCfAfcagaGfaGfaggcu | | AGAGCCUCUCUCUGUGACAUG | |
| | uL96 | | scsu | | UG | |
| AD-1639282 | usgsugacAfuGfUfGfacucuguca | | asUfsgacAfgAfGfucacAfuGfucaca | | UCUGUGACAUGUGACUCUGUC | |
| | uL96 | | sgsa | | AA | |
| AD-1639295 | uscsugucAfaUfUfGfaagacagga | | asUfsccuGfuCfUfucaaUfuGfacaga | | ACUCUGUCAAUUGAAGACAG | |
| | uL96 | | sgsu | | GAC | |
| AD-1639304 | usgsaagaCfaGfGfAfcauuaaaag | | asCfsuuuUfaAfUfguccUfgUfcuuc | | AUUGAAGACAGGACAUUAAA | |
| | uL96 | | asasu | | AGA | |
| AD-1639313 | gsascauuAfaAfAfGfagagcgaga | | asUfscucGfcUfCfucuuUfuAfaugu | | AGGACAUUAAAAGAGAGCGA | |
| | uL96 | | cscsu | | GAG | |
| AD-1639321 | asgscgagAfgAfGfAfgaaacaguu | | asAfsacuGfuUfUfcucuCfuCfucgc | | AGAGCGAGAGAGAGAAACAG | |
| | uL96 | | uscsu | | UUC | |
| AD-1639332 | gsasaacaGfuUfCfAfgauuacugc | | asGfscagUfaAfUfcugaAfcUfguuu | | GAGAAACAGUUCAGAUUACU | |
| | uL96 | | csusc | | GCA | |
| AD-1639342 | asgsauuaCfuGfCfAfcauguggau | | asAfsuccAfcAfUfgugcAfgUfaauc | | UCAGAUUACUGCACAUGUGG | |
| | uL96 | | usgsa | | AUA | |
| AD-1639362 | gsusgguuCfaAfAfAfugccauuuu | | asAfsaaaUfgGfCfauuuUfgAfaccac | | GAGUGGUUCAAAAUGCCAUU | |
| | uL96 | | susc | | UUU | |
| AD-1639373 | usgsccauUfuUfUfGfcacaguguu | | asAfsacaCfuGfUfgcaaAfaAfuggca | | AAUGCCAUUUUUGCACAGUG | |
| | uL96 | | susu | | UUA | |
| AD-1639387 | asgsuguuAfgGfAfAfuuacaaaau | | asAfsuuuUfgUfAfauucCfuAfacac | | ACAGUGUUAGGAAUUACAAA | |
| | uL96 | | usgsu | | AUC | |
| AD-1639396 | asasuuacAfaAfAfUfccacagaagu | | asCfsuucUfgUfGfgauuUfuGfuaau | | GGAAUUACAAAAUCCACAGA | |
| | L96 | | uscsc | | AGA | |
| AD-1639407 | cscsacagAfaGfAfUfguuacuugc | | asGfscaaGfuAfAfcaucUfuCfugug | | AUCCACAGAAGAUGUUACUU | |
| | uL96 | | gsasu | | GCA | |
| AD-1639429 | gsgsgcagAfuCfUfUfaaauaaaau | | asAfsuuuUfaUfUfuaagAfuCfugcc | | CUGGGCAGAUCUUAAAUAAA | |
| | uL96 | | csasg | | AUU | |
| AD-1639437 | asusucaaAfcUfCfUfacuucuguu | | asAfsacaGfaAfGfuagaGfuUfugaa | | AAAUUCAAACUCUACUUCUGU | |
| | uL96 | | ususu | | UG | |
| AD-1639446 | csusacuuCfuGfUfUfgucuaguau | | asAfsuacUfaGfAfcaacAfgAfaguag | | CUCUACUUCUGUUGUCUAGUA | |
| | uL96 | | sasg | | UG | |
| AD-1639460 | usasguauGfuUfAfUfugagcuaau | | asAfsuuaGfcUfCfaauaAfcAfuacua | | UCUAGUAUGUUAUUGAGCUA | |
| | uL96 | | sgsa | | AUG | |
| AD-1639469 | asusugagCfuAfAfUfgauucauug | | asCfsaauGfaAfUfcauuAfgCfucaau | | UUAUUGAGCUAAUGAUUCAU | |
| | uL96 | | sasa | | UGG | |
| AD-1639485 | asusugggAfaAfAfUfaccuuuuua | | asUfsaaaAfaGfGfuauuUfuCfccaau | | UCAUUGGGAAAAUACCUUUU | |
| | uL96 | | sgsa | | UAU | |
| AD-1639495 | csusuuuuAfuAfCfUfccuuuauca | | asUfsgauAfaAfGfgaguAfuAfaaaa | | ACCUUUUUAUACUCCUUUAUC | |
| | uL96 | | gsgsu | | AU | |
| AD-1639505 | uscscuuuAfuCfAfUfgguacugua | | asUfsacaGfuAfCfcaugAfuAfaagga | | ACUCCUUUAUCAUGGUACUGU | |
| | uL96 | | sgsu | | AA | |
| AD-1639521 | usgsuaacUfgUfAfUfccauauuau | | asAfsuaaUfaUfGfgauaCfaGfuuaca | | ACUGUAACUGUAUCCAUAUU | |
| | uL96 | | sgsu | | AUA | |
| AD-1639526 | ususauguCfcAfAfGfugcccacgu | | asAfscguGfgGfCfacuuGfgAfcaua | | UUUUAUGUCCAAGUGCCCACG | |
| | uL96 | | asasa | | UG | |
| AD-1639541 | csascgugAfaUfUfUfgcuggugaa | | asUfsucaCfcAfGfcaaaUfuCfacgug | | CCCACGUGAAUUUGCUGGUGA | |
| | uL96 | | sgsg | | AA | |
| AD-1639550 | ususgcugGfuGfAfAfaguuagcac | | asGfsugcUfaAfCfuuucAfcCfagcaa | | AUUUGCUGGUGAAAGUUAGC | |
| | uL96 | | sasu | | ACU | |
| AD-1639559 | asasaguuAfgCfAfCfuugugugua | | asUfsacaCfaCfAfagugCfuAfacuuu | | UGAAAGUUAGCACUUGUGUG | |
| | uL96 | | scsa | | UAA | |
| AD-1639568 | ascsuuguGfuGfUfAfaauucuacu | | asAfsguaGfaAfUfuuacAfcAfcaag | | GCACUUGUGUGUAAAUUCUA | |
| | uL96 | | usgsc | | CUU | |
| AD-1639577 | usasaauuCfuAfCfUfuccucuugu | | asAfscaaGfaGfGfaaguAfgAfauuu | | UGUAAAUUCUACUUCCUCUUG | |
| | uL96 | | ascsa | | UG | |
| AD-1639591 | csusugugUfgUfUfUfuaccaagua | | asUfsacuUfgGfUfaaaaCfaCfacaag | | CUCUUGUGUGUUUUACCAAG | |
| | uL96 | | sasg | | UAU | |
| AD-1639599 | ususuaccAfaGfUfAfuuuauacuc | | asGfsaguAfuAfAfauacUfuGfguaa | | GUUUUACCAAGUAUUUAUAC | |
| | uL96 | | asasc | | UCU | |
| AD-1639615 | ascsucugGfuGfCfAfacuaacuac | | asGfsuagUfuAfGfuugcAfcCfagag | | AUACUCUGGUGCAACUAACUA | |
| | uL96 | | usasu | | CU | |
| AD-1639628 | usasacuaCfuGfUfGfugaggaauu | | asAfsauuCfcUfCfacacAfgUfaguua | | ACUAACUACUGUGUGAGGAA | |
| | uL96 | | sgsu | | UUG | |
| AD-1639642 | gsgsaauuGfgUfCfCfaugugcaau | | asAfsuugCfaCfAfuggaCfcAfauucc | | GAGGAAUUGGUCCAUGUGCA | |
| | uL96 | | susc | | AUA | |
| AD-1639655 | gsusgcaaUfaAfAfUfaccaaugaa | | asUfsucaUfuGfGfuauuUfaUfugca | | AUGUGCAAUAAAUACCAAUG | |
| | uL96 | | csasu | | AAG | |

TABLE 6
Reporter Screen for Human ADRB1 in Hepa1-6 Cells

RLuc/FLuc
10 nM

| | % | |
|---|---|---|
| | Message | |
| Duplex Name | Remaining | SD |
| AD-1639655.1 | 36.084 | 2.929 |
| AD-1639642.1 | 42.211 | 1.351 |
| AD-1639628.1 | 34.874 | 1.542 |
| AD-1639615.1 | 35.502 | 2.252 |
| AD-1639599.1 | 29.675 | 2.190 |
| AD-1639591.1 | 40.543 | 1.627 |
| AD-1639577.1 | 31.585 | 2.243 |
| AD-1639568.1 | 32.152 | 2.750 |
| AD-1639559.1 | 30.767 | 2.016 |
| AD-1639550.1 | 30.100 | 1.316 |
| AD-1639541.1 | 54.998 | 0.655 |
| AD-1639526.1 | 60.064 | 6.239 |
| AD-1639521.1 | 38.648 | 2.606 |
| AD-1639505.1 | 33.810 | 2.120 |
| AD-1639495.1 | 30.011 | 3.407 |
| AD-1639485.1 | 33.311 | 1.239 |
| AD-1639469.1 | 30.608 | 3.449 |
| AD-1639460.1 | 28.107 | 1.807 |
| AD-1639446.1 | 32.926 | 1.836 |
| AD-1639437.1 | 35.594 | 0.996 |
| AD-1639429.1 | 36.858 | 0.515 |
| AD-1639407.1 | 27.111 | 1.887 |
| AD-1639396.1 | 26.091 | 0.591 |
| AD-1639387.1 | 23.177 | 1.067 |
| AD-1639373.1 | 28.514 | 1.069 |
| AD-1639362.1 | 23.057 | 1.017 |
| AD-1639342.1 | 23.441 | 1.056 |
| AD-1639332.1 | 39.829 | 1.991 |
| AD-1639321.1 | 42.864 | 3.218 |
| AD-1639313.1 | 27.956 | 1.076 |
| AD-1639304.1 | 27.695 | 1.370 |
| AD-1639295.1 | 46.853 | 1.375 |
| AD-1639282.1 | 46.448 | 3.809 |
| AD-1639272.1 | 51.909 | 4.028 |
| AD-1639263.1 | 52.664 | 2.318 |
| AD-1639240.1 | 28.741 | 1.724 |
| AD-1639226.1 | 28.551 | 1.140 |
| AD-1639220.1 | 30.348 | 1.401 |
| AD-1639206.1 | 50.474 | 3.902 |
| AD-1639192.1 | 55.729 | 3.739 |
| AD-1639183.1 | 53.680 | 1.718 |
| AD-1639174.1 | 71.248 | 4.148 |
| AD-1639157.1 | 58.249 | 1.688 |
| AD-1639145.1 | 59.585 | 1.125 |
| AD-1639143.1 | 57.108 | 3.631 |
| AD-1639133.1 | 39.929 | 1.668 |
| AD-1639123.1 | 38.089 | 0.975 |
| AD-1639116.1 | 53.771 | 2.538 |
| AD-1639108.1 | 54.713 | 1.465 |
| AD-1639099.1 | 33.124 | 1.104 |
| AD-1639087.1 | 17.831 | 0.919 |
| AD-1639077.1 | 21.644 | 1.218 |
| AD-1639068.1 | 34.199 | 1.527 |
| AD-1639057.1 | 30.434 | 0.612 |
| AD-1639044.1 | 21.603 | 1.077 |
| AD-1639034.1 | 44.290 | 0.840 |
| AD-1639025.1 | 54.566 | 3.721 |
| AD-1639016.1 | 77.977 | 2.892 |
| AD-1639007.1 | 66.117 | 4.314 |
| AD-1638994.1 | 82.085 | 6.808 |
| AD-1638985.1 | 94.681 | 4.373 |
| AD-1638971.1 | 39.182 | 3.350 |
| AD-1638968.1 | 82.886 | 5.744 |
| AD-1638961.1 | 71.673 | 6.710 |
| AD-1638943.1 | 50.576 | 1.865 |
| AD-1638933.1 | 25.070 | 1.640 |
| AD-1638927.1 | 29.333 | 1.531 |
| AD-1638911.1 | 34.023 | 1.142 |
| AD-1638902.1 | 87.193 | 1.563 |
| AD-1638890.1 | 87.995 | 4.106 |
| AD-1638886.1 | 33.282 | 1.122 |
| AD-1638876.1 | 69.635 | 4.698 |
| AD-1638859.1 | 40.500 | 3.358 |
| AD-1638838.1 | 52.523 | 2.748 |
| AD-1638823.1 | 50.377 | 1.268 |
| AD-1638813.1 | 45.052 | 3.079 |
| AD-1638803.1 | 62.258 | 2.853 |
| AD-1638794.1 | 54.294 | 1.919 |
| AD-1638780.1 | 47.039 | 2.699 |
| AD-1638771.1 | 50.779 | 4.235 |
| AD-1638762.1 | 34.324 | 2.813 |
| AD-1638752.1 | 32.510 | 2.607 |
| AD-1638732.1 | 63.193 | 3.476 |
| AD-1638705.1 | 87.981 | 8.265 |
| AD-1638678.1 | 82.539 | 8.707 |
| AD-1638666.1 | 79.422 | 6.876 |
| AD-1638662.1 | 86.689 | 6.919 |
| AD-1638659.1 | 73.164 | 6.691 |
| AD-1638640.1 | 67.176 | 5.555 |
| AD-1638631.1 | 60.178 | 2.869 |
| AD-1638610.1 | 82.033 | 7.435 |
| AD-1638597.1 | 67.660 | 4.963 |
| AD-1638585.1 | 73.917 | 3.520 |
| AD-1638576.1 | 57.774 | 6.457 |
| AD-1638561.1 | 76.720 | 5.989 |
| AD-1638549.1 | 72.374 | 3.388 |
| AD-1638539.1 | 76.908 | 6.768 |
| AD-1638529.1 | 62.888 | 5.852 |
| AD-1638500.1 | 55.562 | 4.231 |
| AD-1638485.1 | 64.806 | 4.092 |
| AD-1638474.1 | 74.497 | 3.329 |
| AD-1638456.1 | 82.647 | 10.910 |
| AD-1638444.1 | 65.271 | 7.489 |
| AD-1638435.1 | 59.200 | 4.952 |
| AD-1638431.1 | 68.084 | 4.786 |
| AD-1638422.1 | 81.550 | 7.527 |
| AD-1638406.1 | 77.835 | 6.358 |
| AD-1638393.1 | 65.579 | 4.797 |
| AD-1638370.1 | 67.546 | 2.641 |
| AD-1638356.1 | 70.953 | 2.786 |
| AD-1638347.1 | 76.982 | 7.255 |
| AD-1638314.1 | 72.999 | 5.056 |
| AD-1638305.1 | 76.084 | 6.443 |
| AD-1638296.1 | 63.672 | 3.251 |
| AD-1638287.1 | 59.750 | 4.653 |
| AD-1638278.1 | 70.698 | 6.732 |
| AD-1638266.1 | 41.929 | 6.152 |
| AD-1638253.1 | 72.481 | 3.612 |
| AD-1638230.1 | 63.118 | 6.706 |
| AD-1638221.1 | 75.254 | 6.239 |
| AD-1638205.1 | 62.400 | 3.774 |
| AD-1638196.1 | 69.535 | 5.021 |
| AD-1638172.1 | 83.596 | 6.016 |
| AD-1638163.1 | 59.876 | 3.051 |
| AD-1638154.1 | 48.625 | 3.199 |
| AD-1638136.1 | 77.997 | 3.698 |
| AD-1638127.1 | 81.215 | 3.826 |
| AD-1638117.1 | 65.762 | 5.370 |
| AD-1638104.1 | 94.098 | 7.149 |
| AD-1638092.1 | 62.706 | 1.669 |
| AD-1638077.1 | 62.318 | 4.690 |
| AD-1638051.1 | 90.974 | 6.574 |
| AD-1638021.1 | 98.145 | 5.195 |
| AD-1637971.1 | 90.755 | 4.262 |
| AD-1637961.1 | 57.582 | 1.207 |
| Positive Control | 1.420 | 0.166 |

### Example 4: SOD1 mRNA Knockdown in Mouse Cardiac and Skeletal Muscle

dsRNA single strands comprising one or more lipophilic moieties (e.g., any compound or chemical moiety having an affinity for lipids, e.g., a C₂₂ hydrocarbon chain) conjugated at various positions on the sense strand targeting mouse Superoxide Dismutase 1 (SOD1) or mouse myostatin (MTSN) were synthesized on a solid support followed by hybridization of complementary strands to form a duplex.

The modified nucleotide sequences of the sense strands used in this Example are provided in Table 8A and the modified nucleotide sequences of the duplexes used in this study are provided in Table 8B.

The effect of the agents in Table 8B was examined in vivo in wild type C57BL/6 mice (6-8 weeks old) as summarized in Table 7. Briefly, animals (n=3) were intravenously administered a single 5 mg/kg or 20 mg/kg dose of the agent on Day 0. On Day 14, animals were sacrificed and livers, heart, and quadriceps were collected.

For SOD1 analysis, RNA was isolated from powdered quadricep and heart with the PerkinElmer Chemagic system according to the supplier's guidelines. Resulting RNA was used to generate cDNA using a High-Capacity cDNA Reverse Transcription Kit (Applied Biosystems, 4368813). RT-qPCR was performed using the LightCycler® 480 Probes Master in the LightCycler®480 Instrument system using gene specific TaqMan assays for each target mouse-specific SOD1 (ThermoFisher Scientific, Mm01344233_g1) and Gapdh (ThermoFisher Scientific, Mm99999915_g1) were used for RT-qPCR analysis. Each sample was analyzed in duplicate by RT-qPCR. Data were analyzed using the ΔΔCt method normalizing to control animals treated with 1×PBS alone.

For MTSN analysis, heart and quadricep powder were homogenized and lysed in 750 μL of QIAzol Lysis Reagent for 5 minutes ×2 at 25 Hz using a TissueLyser. Chloroform (150 μL) was thoroughly mixed with each sample and lysates were centrifugated at full speed for 15 minutes at 4° C. Total RNA was isolated from the supernatant using the RNeasy® 96 Universal Tissue Kit. Resulting RNA (1500 ng) was used for cDNA synthesis using the Applied Biosystems™ High-Capacity cDNA Reverse Transcription Kit with RNase Inhibitor. For qPCR reactions, two μl of cDNA and 10 μl Fast Advanced Mastermix (ThermoFisher Scientific A44359) were added to 1× house-keeping probe and 1× target probe per well. Real time PCR was done in a QuantStudio5 Real Time PCR system (ThermoFisher Scientific). The mean level of SOD1 mRNA was normalized to the mean level of GAPDH mRNA for each sample. Group mean values were then normalized to the mean value for the PBS treated group, to obtain the relative level of SOD1 mRNA expression.

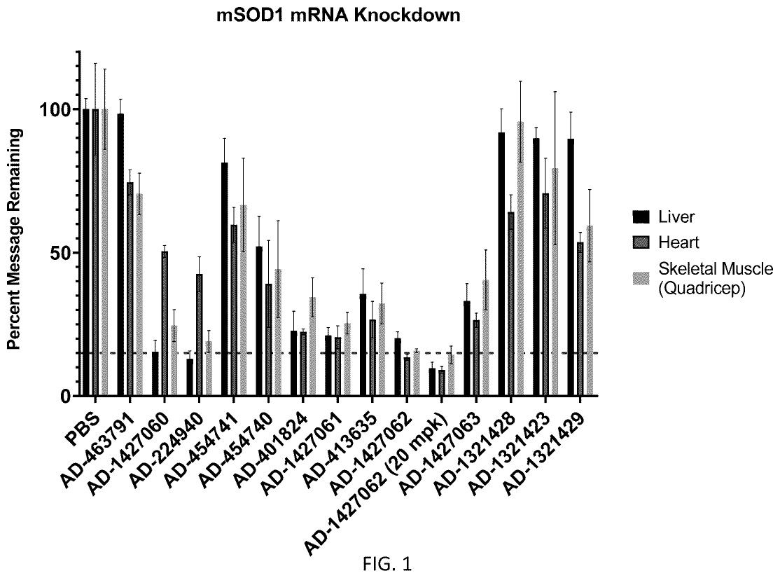

The results are shown in FIG. 1 and demonstrate that an increase in chain length of the lipophilic moiety on the sense strand at position 6 shows a strong positive correlation with SOD1 knockdown in the heart and skeletal muscle. These data also demonstrate that the most potent knockdown was observed in muscle tissue via intramuscular administration of a C₂₂ hydrocarbon chain conjugated to the sense strand at position 6 of the dsRNA agent as evidenced by the potent inhibition of AD-1427062.

TABLE 7
Study Design

| | | # | Delivery | Dose |
|---|---|---|---|---|
| Group | Duplex ID | Animals | Route | (mg/kg) |
| 1 | PBS | 3 | IV | 5 |
| 2 | AD-463791 | 3 | IV | 5 |
| 3 | AD-1427060 | 3 | IV | 5 |
| 4 | AD-224940 | 3 | IV | 5 |
| 5 | AD-454741 | 3 | IV | 5 |
| 6 | AD-454740 | 3 | IV | 5 |
| 7 | AD-401824 | 3 | IV | 5 |
| 8 | AD-1427061 | 3 | IV | 5 |
| 9 | AD-413635 | 3 | IV | 5 |
| 10 | AD-1427062 | 3 | IV | 5 |
| 11 | AD-1427062 | 3 | IV | 20 |
| 12 | AD-1427063 | 3 | IV | 5 |
| 13 | AD-1321428 | 3 | IV | 5 |
| 14 | AD-1321423 | 3 | IV | 5 |
| 15 | AD-1321429 | 3 | IV | 5 |

TABLE 8A
siRNA Sense Strands Synthesized for in vivo Studies

| | | | | Molecular | Molecular |
|---|---|---|---|---|---|
| Duplex ID | Strand | Target | Oligonucleotide Sequence 5′ to 3′ | weight | weight found |
| A-899929 | sense | SOD1 | csasuuuuAfaUfCfCfucacucuasasa | 6833.579 | 6830.02 |
| A-2219787 | sense | SOD1 | csasuuuY118AfaUfCfCfucacucuasasa | 7444.543 | 7440.52 |
| A-444399 | sense | SOD1 | csasuuuuAfaUfCfCfucacucuaaaL10 | 7506.365 | 7502.51 |
| A-637445 | sense | SOD1 | csasuuu(Ude)AfaUfCfCfucacucuasasa | 6959.826 | 6956.16 |
| A-637447 | sense | SOD1 | csasuuu(Utd)AfaUfCfCfucacucuasasa | 7015.926 | 7012.22 |
| A-637448 | sense | SOD1 | csasuuu(Uhd)AfaUfCfCfucacucuasasa | 7043.976 | 7040.25 |
| A-2356923 | sense | SOD1 | csasuuu(Uod)AfaUfCfCfucacucuasasa | 7072.031 | 7068.28 |
| A-637449 | sense | SOD1 | csasuuu(Uol)AfaUfCfCfucacucuasasa | 7070.026 | 7066.26 |
| A-2578434 | sense | SOD1 | csasuuu(Uda)AfaUfCfCfucacucuasasa | 7128.132 | 7124.35 |
| A-2248662 | sense | SOD1 | csasuuuY 158AfaUfCfCfucacucuasasa | 7121.081 | 7117.25 |
| A-2219771 | sense | SOD1 | csasuuu(Uh)AfaUfCfCfucac(Uh)cuasasa | 6973.84 | 6970.17 |
| A-2219766 | sense | SOD1 | csasuuu(Uh)AfaUfCfCfucac(Uh)cuasas(Ah) | 7043.978 | 7040.25 |
| A-2219772 | sense | SOD1 | (Chs)asuuu(Uh)AfaUfCfCfucac(Uh)(Ch)uasas(Ah) | 7184.246 | 7180.41 |
| A-3340763 | sense | SOD1 | ususggg(Cda)AfaAfGfGfuggaaaugsasa | 7468.423 | 7464.473 |
| A-3013336 | sense | Mstn | asusggc(Ada)AfaGfAfAfcaaauaausasa | 7387.441 | 7385.31 |

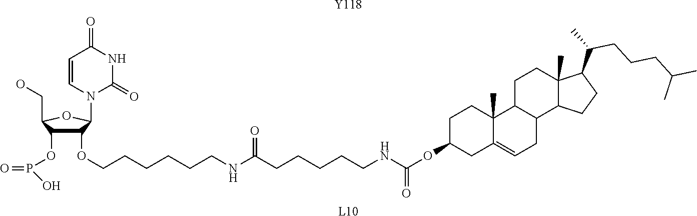

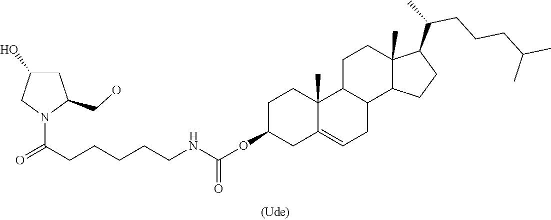 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C]

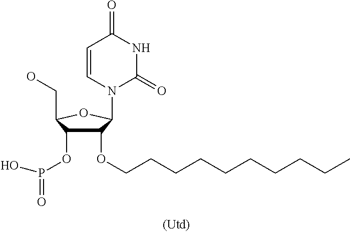

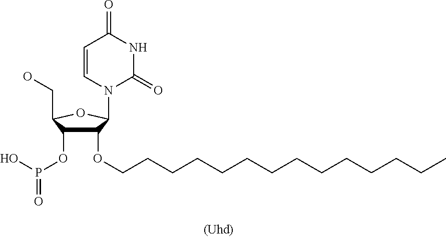

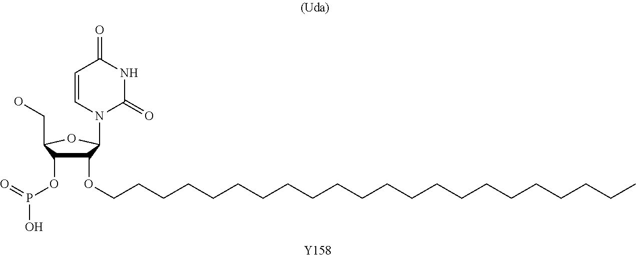

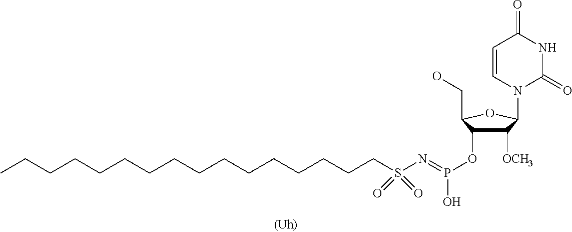

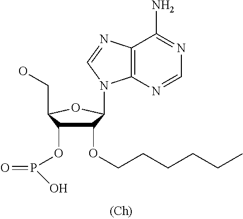

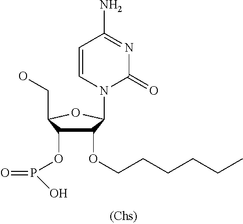

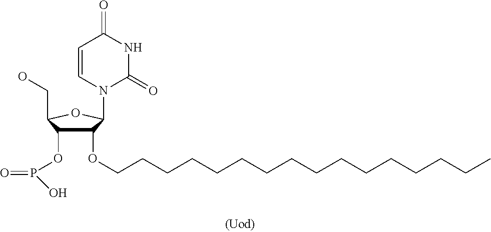

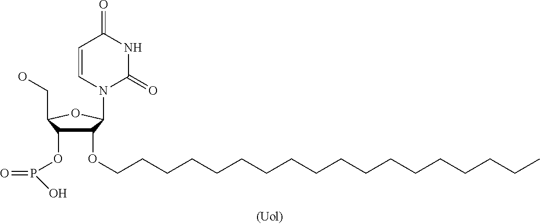

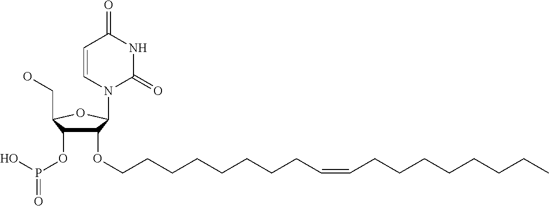

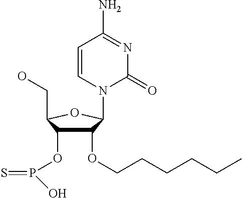

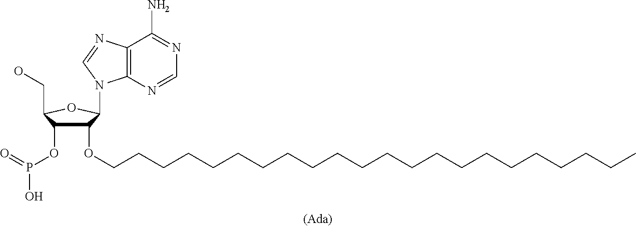

TABLE 8B
siRNA Duplexes Synthesized for in vivo Studies

| | | | | | | Molecular |
|---|---|---|---|---|---|---|
| | | | | | Molecular | Weight |
| Duplex ID | Oligo ID | Strand | Target | Sequence 5′ to 3′ | Weight | Found |
| AD-463791 | A-899929 | sense | SOD1 | csasuuuuAfaUfCfCfucacucuasasa | 6833.579 | 6830.02 |
| | A-444402 | antis | SOD1 | VPusUfsuagAfgUfGfaggaUfuAfaaaugsasg | 7851.156 | 7847.15 |
| AD-1427060 | A-2219787 | sense | SOD1 | csasuuuY118AfaUfCfCfucacucuasasa | 7444.543 | 7440.52 |
| | A-444402 | antis | SOD1 | VPusUfsuagAfg UfGfaggaUfuAfaaaugsasg | 7851.156 | 7847.15 |
| AD-224940 | A-444399 | sense | SOD1 | csasuuuuAfaUfCfCfucacucuaaaL10 | 7506.365 | 7502.52 |
| | A-444402 | antis | SOD1 | VPusUfsuagAfgUfGfaggaUfuAfaaaugsasg | 7851.156 | 7847.15 |
| AD-454741 | A-637445 | sense | SOD1 | csasuuu(Ude)AfaUfCfCfucacucuasasa | 6959.826 | 6956.16 |
| | A-444402 | antis | SOD1 | VPusUfsuagAfgUfGfaggaUfuAfaaaugsasg | 7851.156 | 7847.15 |
| AD-454740 | A-637447 | sense | SOD1 | csasuuu(Utd)AfaUfCfCfucacucuasasa | 7015.926 | 7012.22 |
| | A-444402 | antis | SOD1 | VPusUfsuagAfgUfGfaggaUfuAfaaaugsasg | 7851.156 | 7847.15 |
| AD-401824 | A-637448 | sense | SOD1 | csasuuu(Uhd)AfaUfCfCfucacucuasasa | 7043.976 | 7040.25 |
| | A-444402 | antis | SOD1 | VPusUfsuagAfgUfGfaggaUfuAfaaaugsasg | 7851.156 | 7847.15 |
| AD-1427061 | A-2356923 | sense | SOD1 | csasuuu(Uod)AfaUfCfCfucacucuasasa | 7072.031 | 7068.28 |
| | A-444402 | antis | SOD1 | VPusUfsuagAfgUfGfaggaUfuAfaaaugsasg | 7851.156 | 7847.15 |
| AD-413635 | A-637449 | sense | SOD1 | csasuuu(Uol)AfaUfCfCfucacucuasasa | 7070.026 | 7066.27 |
| | A-444402 | antis | SOD1 | VPusUfsuagAfgUfGfaggaUfuAfaaaugsasg | 7851.156 | 7847.15 |
| AD-1427062 | A-2578434 | sense | SOD1 | csasuuu(Uda)AfaUfCfCfucacucuasasa | 7128.132 | 7124.34 |
| | A-444402 | antis | SOD1 | VPusUfsuagAfgUfGfaggaUfuAfaaaugsasg | 7851.156 | 7847.15 |
| AD-1427063 | A-2248662 | sense | SOD1 | csasuuuY158AfaUfCfCfucacucuasasa | 7121.081 | 7117.24 |
| | A-444402 | antis | SOD1 | VPusUfsuagAfgUfGfaggaUfuAfaaaugsasg | 7851.156 | 7847.15 |
| AD-1321428 | A-2219771 | sense | SOD1 | csasuuu(Uh)AfaUfCfCfucac(Uh)cuasasa | 6973.84 | 6970.17 |
| | A-444402 | antis | SOD1 | VPusUfsuagAfgUfGfaggaUfuAfaaaugsasg | 7851.156 | 7847.15 |
| AD-1321423 | A-2219766 | sense | SOD1 | csasuuu(Uh)AfaUfCfCfucac(Uh)cuasas(Ah) | 7043.978 | 7040.25 |
| | A-444402 | antis | SOD1 | VPusUfsuagAfgUfGfaggaUfuAfaaaugsasg | 7851.156 | 7847.15 |
| AD-1321429 | A-2219772 | sense | SOD1 | (Chs)asuuu(Uh)AfaUfCfCfucac(Uh)(Ch)uasas(Ah) | 7184.246 | 7180.41 |
| | A-444402 | antis | SOD1 | VPusUfsuagAfgUfGfaggaUfuAfaaaugsasg | 7851.156 | 7847.15 |
| AD-1640773 | A-3013336 | sense | Mstn | asusggc(Ada)AfaGfAfAfcaaauaausasa | 7387.441 | 7385.31 |
| | A-135655 | antis | Mstn | VPusUfsauuAfuUfUfguucUfuUfgccaususa | 7533.956 | 7530.85 |
| AD-1812376 | A-3340763 | sense | SOD1 | ususggg(Cda)AfaAfGfGfuggaaaugsasa | | |
| | A-859296 | antis | SOD1 | VPusUfscauUfuCfCfaccuUfuGfcccaasgsu | | |
| AD-1615344 | A-2894724 | sense | MALAT1 | asascga(Cda)UfgGfAfGfuaugauuasasa | 7373.359 | 7369.449 |
| | A-1850572 | antis | MALAT1 | VPusUfsuaaucauacucCfaGfucguususc | 7593.999 | 7590.091 |
| AD-1615345 | A-2225899 | sense | MALAT1 | cscsgcugCfuAfUfUfagaaugcasusa | 6967.675 | 6964.065 |
| | A-2894725 | antis | MALAT1 | VPusAfsugcauucuaauAfg(Cda)agcggsgsa | 8104.808 | 8100.55 |

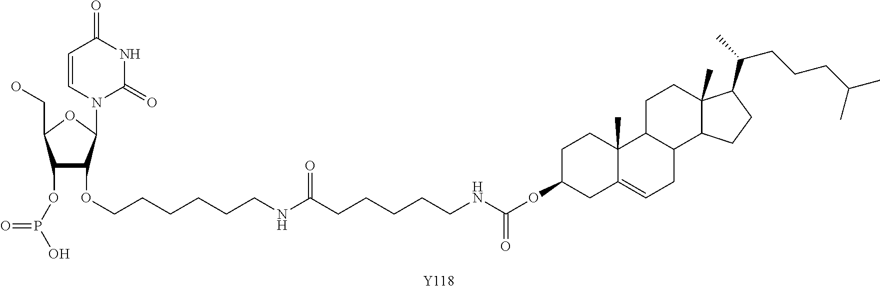

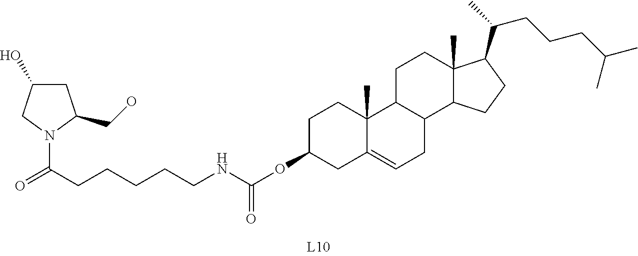 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C]

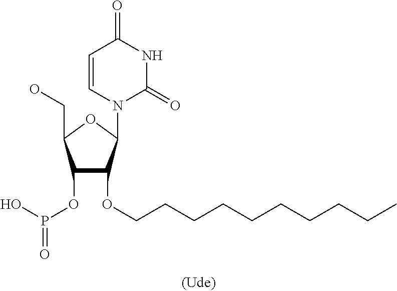

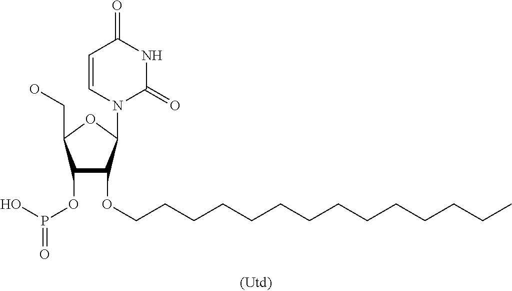

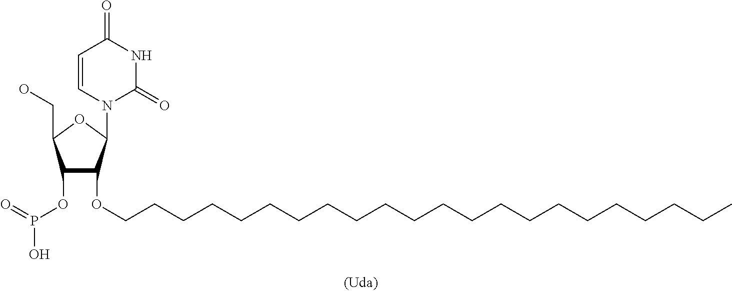

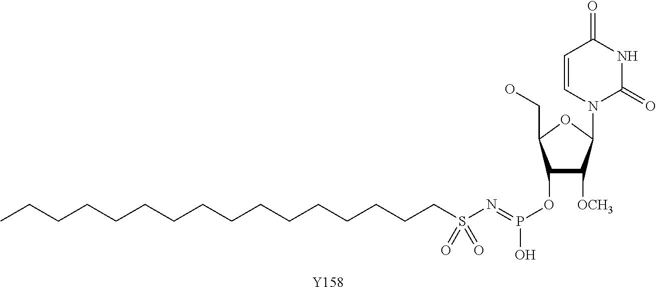

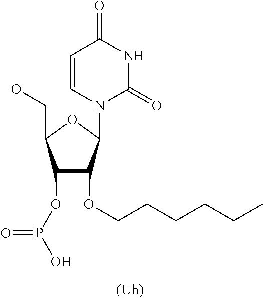

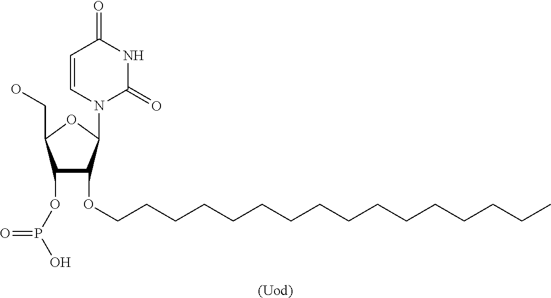

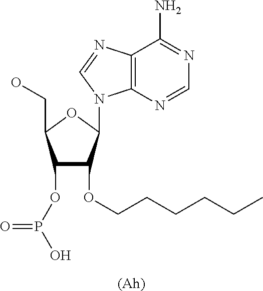

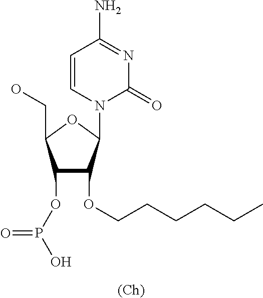

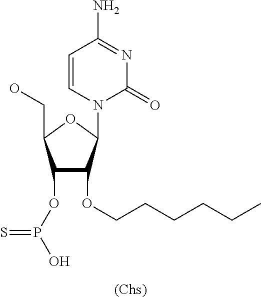

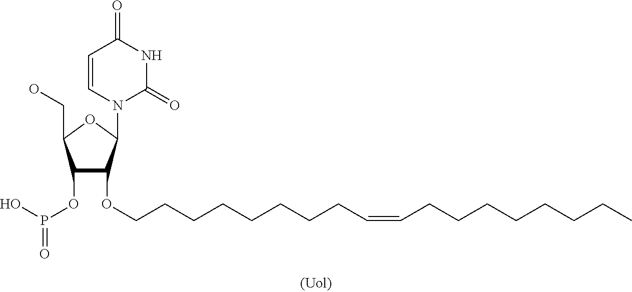

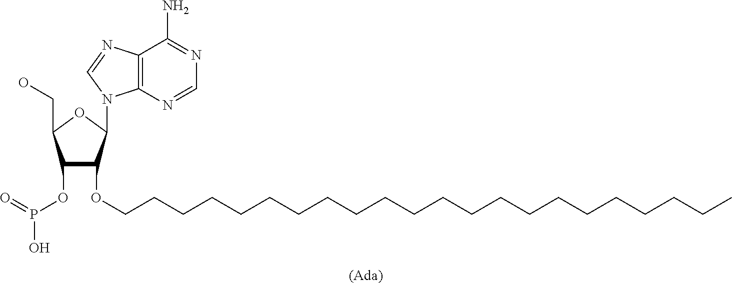

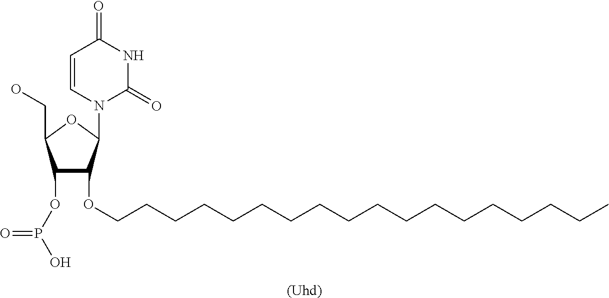

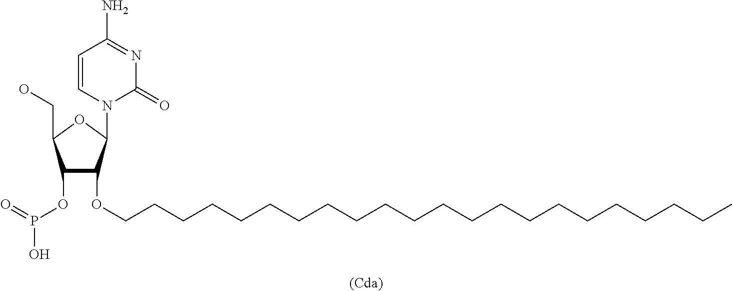

### Example 5: MALAT1 and SOD1 mRNA Knockdown in Mouse Cardiac and Skeletal Muscle

MALAT1 (metastasis associated lung adenocarcinoma transcript 1) and SOD1 (superoxide dismutase type 1) gene silencing in cardiac and skeletal muscle was studied with dsRNA agents comprising one or more C22 hydrocarbon chains conjugated to position 6 on the sense strand, counting from the 5′-end of the sense strand.

dsRNA single strands comprising one or more C22 hydrocarbon chains conjugated to position 6 on the sense strand, counting from the 5′-end of the sense strand, targeting mouse MALAT1 or mouse SOD1 were synthesized on a solid support followed by hybridization of complementary strands to form a duplex.

The modified nucleotide sequences of the duplexes used in this study are provided in Table 7.

The effect of these was examined in vivo in wild type C57BL/6 mice (6-8 weeks old) as summarized in Table 9. Animals (n=3) were intravenously administered a single 1 mg/kg, 5 mg/kg, or 20 mg/kg dose of the agent on Day 0. On Day 14 or Day 28, animals were sacrificed and tissues (e.g., livers, heart, and quadriceps) were collected and the level of MALAT1 mRNA or SOD1 mRNA was determined by quantitative RT-PCR.

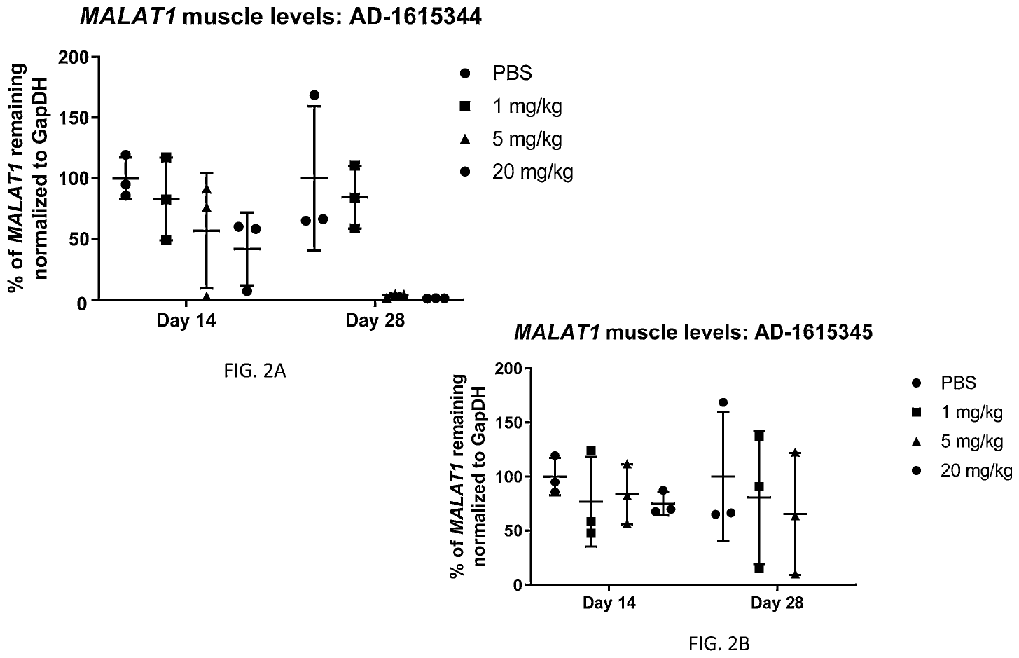

The results of the effect of AD-1615344 and AD-1615345 on MALAT1 expression in skeletal muscle tissue are shown in FIGS. 2A and 2B, respectively, and demonstrate that at Day 28 post-dose the duplex AD-1615344, comprising a C₂₂ hydrocarbon chain conjugated to the sense strand at position 6, counting from the 5′-end of the sense strand, potently inhibits MALAT1 expression in skeletal muscle tissue.

TABLE 9
Study Design

| Group | N | siRNA | I.V. dose | | sac | tissues |
|---|---|---|---|---|---|---|
| 1 | 3 | PBS | NA | | D 14 | Quad, heart, |
| | | | | | | liver, kidney |
| 2 | | AD-1615344 | 1 | mg/kg | | Quad, heart |
| 3 | | AD-1615345 | | | | |
| 4 | | AD-1615344 | 5 | mg/kg | | |
| 5 | | AD-1615345 | | | | |
| 6 | | AD-1427062 | | | | Quad, heart, |
| | | | | | | liver, kidney |
| 7 | | AD-1615344 | 20 | mg/kg | | Quad, heart |
| 8 | | AD-1615345 | | | | |
| 9 | | PBS | NA | | D 28 | Quad, heart, |
| | | | | | | liver, kidney |
| 10 | | AD-1615344 | 1 | mg/kg | | Quad, heart |
| 11 | | AD-1615345 | | | | |
| 12 | | AD-1615344 | 5 | mg/kg | | |
| 13 | | AD-1615345 | | | | |
| 14 | | AD-1427062 | | | | Quad, heart, |
| | | | | | | liver, kidney |
| 15 | | AD-1615344 | 20 | mg/kg | | Quad, heart |

| | |
|---|---|
| 16 | AD-1615345 |

### Example 6: MSTN1 mRNA Knockdown in Mouse Skeletal Muscle and SOD1 mRNA Knockdown in Mouse Skeletal Muscle, Mouse Cardiac Muscle, and Mouse Adipose Tissue

The effect of route of administration on mouse myostatin (MSTN1) gene knockdown in mouse skeletal muscle and the effect of route of administration on mouse superoxide dismutase 1 (SOD1) knockdown in mouse skeletal muscle, mouse cardiac muscle, and mouse adipose tissue by dsRNA agents comprising one or more C22 hydrocarbon chains conjugated to position 6 on the sense strand, counting from the 5′-end of the sense strand were examined.

Briefly, a single 5 mg/kg dose of AD-1640773, targeting the mouse MSTN1 gene, or AD-1427062, targeting the mouse SOD1 gene, was administered to wild type C57BL/6 mice (6-8 weeks old) intravenously (IV), subcutaneously (SQ), or intramuscularly (IM) at Day 0. For the IM groups, the dsRNA agent was only administered in the left quadricep. A single 5 mg/kg dose of AD-1427062 was also administered to wild type C57BL/6 mice (6-8 weeks old) intraperitoneally (IP). PBS was intravenously administered as a control. At Day 21, animals were sacrificed, tissue including, quadricep muscle tissue from both quadriceps (left quadricep, IM injected (IM (I) and right quadricep, IM distal, not injected (IM-(D)), cardiac muscle tissue, and gonadal adipose tissue, was collected, and the level of MSTN1 or SOD1 mRNA was determined by quantitative RT-PCR.

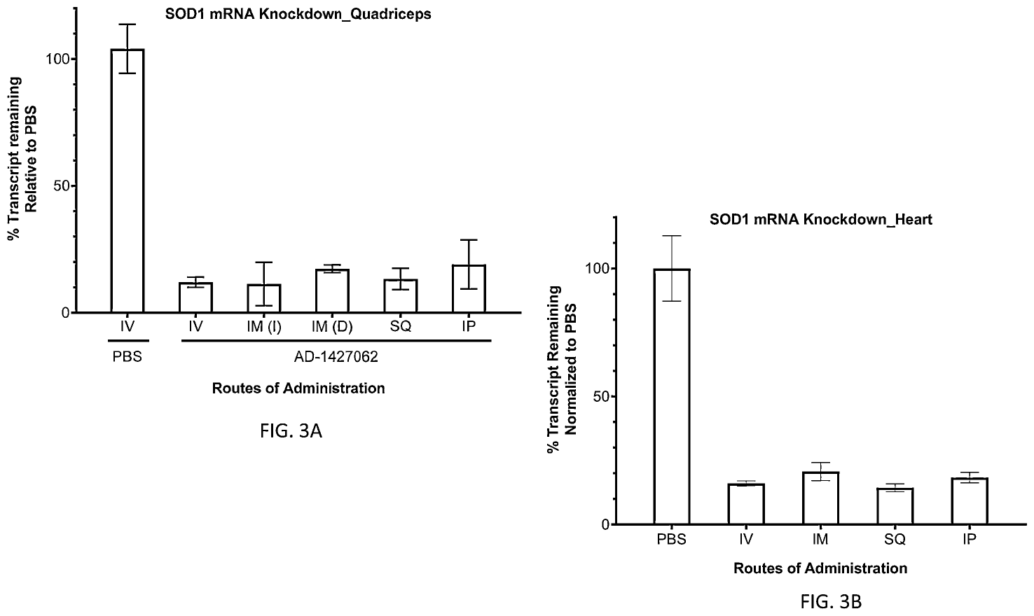

The results of these analyses are provided in FIGS. 3A-3E.

In FIG. 3A, the data demonstrate that, although IM administration of AD-1427062 shows slightly better knockdown of SOD1 mRNA in the injected skeletal muscle, this observed knockdown is comparable to the knockdown of SOD1 mRNA observed in the distal muscle tissue and to the knockdown of SOD1 mRNA in skeletal muscle observed following administration of AD-1640773 intravenously, subcutaneously, or intraperitoneally.

FIG. 3B demonstrates that the route of administration of AD-1427062 does not affect the ability of dsRNA agents comprising one or more C₂₂ hydrocarbon chains conjugated to position 6 on the sense strand, counting from the 5′-end of the sense strand to potently knockdown SOD1 mRNA in cardiac muscle tissue.

Although the basal level of myostatin is extremely variable and all routes of administration of AD-1640773 knocked down MTSN mRNA levels, FIG. 3C demonstrates that IM administration of AD-1640773 provides the most potent knockdown of MTSN mRNA in skeletal muscle tissue.

FIGS. 3D and 3E demonstrate that the route of administration of AD-1427062 does not affect the ability of dsRNA agents comprising one or more C₂₂ hydrocarbon chains conjugated to position 6 on the sense strand, counting from the 5′-end of the sense strand to potently knockdown SOD1 mRNA in adipose tissue.

### Example 7: Dose Response of SOD1 mRNA Knockdown in Mouse Adipose Tissue Intravenously (IV) Administered SOD1 siRNA at Days 14 and 28 Post-Dose

The effect of dsRNA agents targeting SOD1 and comprising one or more C22 hydrocarbon chains conjugated to position 6 on the sense strand, counting from the 5′-end of the sense strand, were examined for knockdown in brown, gonadal, and subcutaneous adipose tissues.

Specifically, an exemplary duplex targeting SOD1, AD-1427062, was examined in vivo in wild type C57BL/6 mice (6-8 weeks old) as summarized in Table 10. Briefly, animals (n=3) were intravenously administered a single 5 mg/kg, 2 or 0.5 mg/kg dose of the dsRNA agent on Day 0. On Days 14 and 28, animals were sacrificed, and brown, gonadal, and subcutaneous adipose tissues were collected.

For SOD1 analysis, RNA was isolated by lysing the tissues in 1 mL of Qiazol in the TissueLyser II two cycles of 20 Hz for 2 minutes. Total RNA was then isolated by using the RNeasy Kit from Qiagen following manufacturer's instructions. The optional DNAse digestion step included in the kit was also performed. Resulting RNA was used to generate cDNA using ThermoFisher Scientific Superscript IV cDNA reverse transcription kit (ThermoFisher Scientific, Cat. No. 11756050). In particular, twenty μl of a ready to use master mix and 11 μl of H₂O per reaction was added to RNA isolated above. Plates were sealed, mixed, and incubated on thermal cycler at 25° C. for 10 min, followed by 42° C. for 10 minutes and inactivation step at 85° C. for 5 minutes. RT-qPCR was performed by using two μl of cDNA and 10p Fast Advanced Mastermix (ThermoFisher Scientific A44359) are added to 1× house-keeping probe (GAPDH, ThermoFisher Scientific, Mm99999915_g1) and 1× target probe (SOD1, Mm01344233_g1) per well. Real time PCR was done in a QuantStudio5 Real Time PCR system (ThermoFisher Scientific). The mean level of SOD1 mRNA was normalized to the mean level of GAPDH mRNA for each sample. Group mean values were then normalized to the mean value for the PBS treated group, to obtain the relative level of SOD1 mRNA expression.

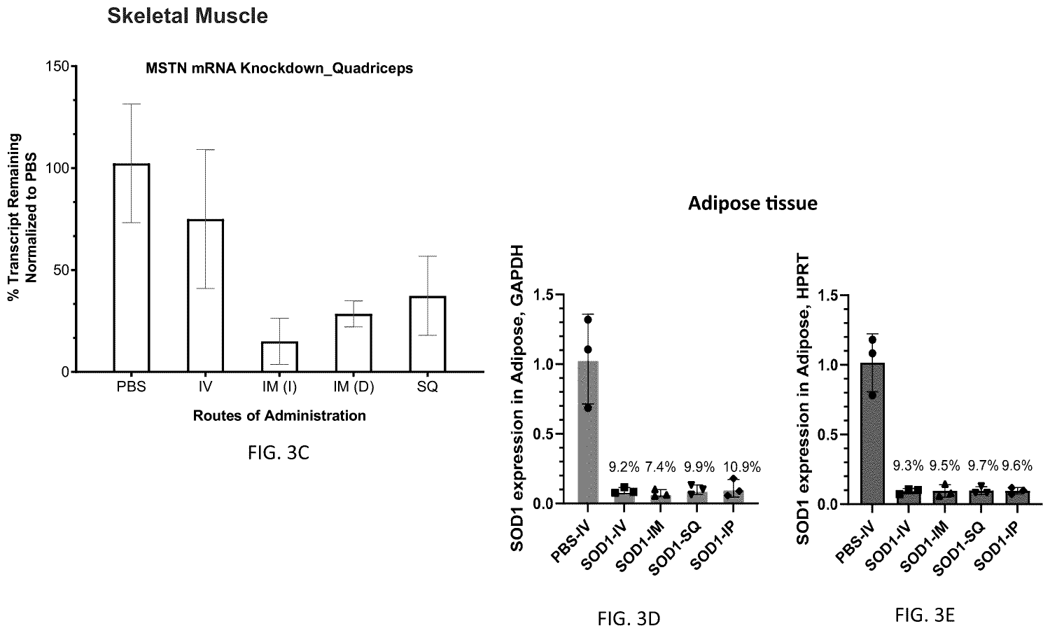

The results are shown in FIGS. 4A-C and demonstrate that a C22 hydrocarbon chain conjugated to the sense strand at position 6 of the dsRNA agent AD-1427062 shows a strong positive correlation with SOD1 knockdown in all adipose tissues analyzed up to day 28 after intravenous injection of a dose as low as 0.5 mg/kg.

TABLE 10
Study Design

| | | # | Delivery | Dose | |
|---|---|---|---|---|---|
| Group | Duplex ID | Animals | Route | (mg/kg) | Day |
| 1 | PBS | 3 | IV | NA | 14 |
| 2 | AD-1427062 | | | 5 | |
| 3 | | | | 2 | |
| 4 | | | | 0.5 | |
| 5 | PBS | | | NA | 28 |
| 6 | AD-1427062 | | | 5 | |
| 7 | | | | 2 | |
| 8 | | | | 0.5 | |

### Example 8: Dose Response of SOD1 mRNA Knockdown in Non-Human Primates (Macaca fascicularis) Adipose Tissue Intravenously Administered SOD1 siRNA at Day 30 Post-Dose

The effect of dsRNA agents targeting SOD1 and comprising one or more C22 hydrocarbon chains conjugated to position 6 on the sense strand, counting from the 5′-end of the sense strand, were examined for knockdown in brown aortic, uterine, subcutaneous and hind limb adipose tissues.

Specifically, an exemplary duplex targeting SOD1, AD-1812376, was examined in vivo in Macaca fascicularis as summarized in Table 11. Briefly, animals (n=3) were intravenously administered a single 3 mg/kg dose of the agent on Day 0. On Day 30, animals were sacrificed, and brown aortic, uterine, subcutaneous and hind limb adipose tissues were collected.

For SOD1 analysis, RNA was isolated by lysing the tissues in 1 mL of Qiazol in the TissueLyser II two cycles of 20 Hz for 2 minutes. Total RNA was then isolated by using the RNeasy Kit from Qiagen following manufacturer's instructions. Resulting RNA was used to generate cDNA using ThermoFisher Scientific Superscript IV cDNA reverse transcription kit (ThermoFisher Scientific, Cat. No. 11756050):

Twenty μl of a ready to use master mix and 11 μl of H₂O per reaction was added to RNA isolated above. Plates were sealed, mixed, and incubated on thermal cycler at 25° C. for 10 min, followed by 42° C. for 10 min and inactivation step at 85° C. for 5 min. RT-qPCR was performed by using two μl of cDNA and 10 μl Fast Advanced Mastermix (ThermoFisher Scientific A44359) are added to 1× house-keeping probe (GAPDH, ThermoFisher Scientific, Mf04392546_g1) and 1× target probe (SOD1, Mf04363557 ml) per well. Real time PCR was done in a QuantStudio5 Real Time PCR system (ThermoFisher Scientific). The mean level of SOD1 mRNA was normalized to the mean level of GAPDH mRNA for each sample. Group mean values were then normalized to the mean value for the PBS treated group, to obtain the relative level of SOD1 mRNA expression.

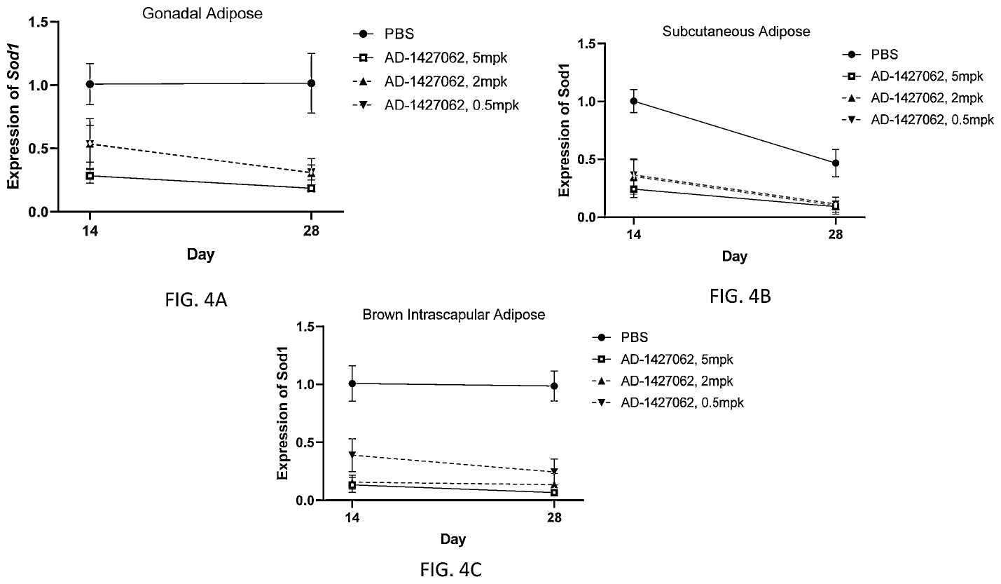

The results are shown in FIG. 5 and demonstrate that a C₂₂ hydrocarbon chain conjugated to the sense strand at position 6 of the dsRNA agent AD-1812376 shows a strong positive correlation with SOD1 knockdown in all adipose tissues analyzed at day 30 post-intravenous injection of SOD siRNA.

TABLE 11
Study Design

| | | # | Delivery | Dose | |
|---|---|---|---|---|---|
| Group | Duplex ID | Animals | Route | (mg/kg) | Day |
| 1 | PBS | 3 | IV | NA | 30 |
| 2 | AD-1812376 | 3 | IV | 3 | 30 |

### Example 9: Effect of dsRNA Agents Targeting Leptin and Comprising One or More C₂₂ Hydrocarbon Chains Conjugated to Position 6 on the Sense Strand, Counting from the 5′-end of the Sense Strand on mRNA Knockdown in Adipose Tissues

Leptin gene silencing in adipose tissue was studied with dsRNA agents comprising one or more C₂₂ hydrocarbon chains conjugated to position 6 on the sense strand, counting from the 5′-end of the sense strand. Leptin is a secreted protein highly enriched in adipose tissue. Knockdown of leptin in adipose tissue will improve metabolic syndrome in mice.

dsRNA agents comprising one or more C22 hydrocarbon chains conjugated to position 6 on the sense strand, counting from the 5′-end of the sense strand, targeting human leptin were synthesized as described above.

The effect of these dsRNA agents was examined in vivo in female and male lean mice (20 weeks old) that were fed with either chow diet or high fat diet, as summarized in Table 12. The modified sense and antisense strand nucleotide sequence of AD-1888031 and AD-1888032 are provided in Table 13.

On study day 0, mice were weighed and then lightly anesthetized under isoflurane and blood was collected via retroorbital collection and processed to serum. Mice were then subcutaneously administered a single 5 mg/kg dose of AD-1888031 or AD-1888032, or PBS control in 10 μl of a solution having a concentration of 0.5 mg/mL. The modified sense and antisense strand nucleotide sequence of AD-1888031 and AD-1888032 are provided in Table 13.

Body weights and blood were then collected at Day 8, Day 14, and Day 21 post-dose to evaluate circulating leptin for each mouse. All serum was collected in the fed state.

TABLE 12
Study Design

| | | | | Subcutaneous | Serum |
|---|---|---|---|---|---|
| Group | Sex | Diet | Treatment | Dose (mg/kg) | Collection |
| 1 | Female | Chow | PBS | NA | Day 0, 8, |
| 2 | Female | Chow | AD-1888031 | 5 | 14, 21 |
| 3 | Female | Chow | AD-1888032 | 5 | |
| 4 | Male | Chow | PBS | NA | |
| 5 | Male | Chow | AD-1888031 | 5 | |
| 6 | Male | Chow | AD-1888032 | 5 | |
| 7 | Male | HFD | AD-1888031 | 5 | |
| 8 | Male | HFD | AD-1888032 | 5 | |

TABLE 13

| | | | | | Molecular |
|---|---|---|---|---|---|
| Duplex | | | | Molecular | Weight |
| ID | Strand | Target | Sequence 5′ to 3′ | Weight | Found |
| AD- | sense | LEP | ususgaa(Gda)UfgUfAfGfuuuuauacsasa | 7289.23 | 7285.35 |
| 1888031 | | | | | |
| | antis | LEP | VPusUfsguaUfaAfAfacuaCfaCfuucaasgsc | 7651.03 | 7647.12 |
| AD- | sense | LEP | gsusgac(Uda)GfgUfUfUfuguuucuasusa | 7235.11 | 7231.28 |
| 1888032 | | | | | |
| | antis | LEP | VPusAfsuagAfaAfCfaaaaCfcAfgucacscsa | 7719.17 | 7715.22 |

Mouse serum leptin levels were measured using the Mouse Serum Leptin Elisa (Crystal Chem, Catalog #90030) following the manufacturer's protocol. All serum samples were measured in duplicate using 5 mL of serum. Serum samples from HFD mice was diluted 1:10 before adding 5_L to the ELISA plate to ensure signal was within the range of the standard curve.

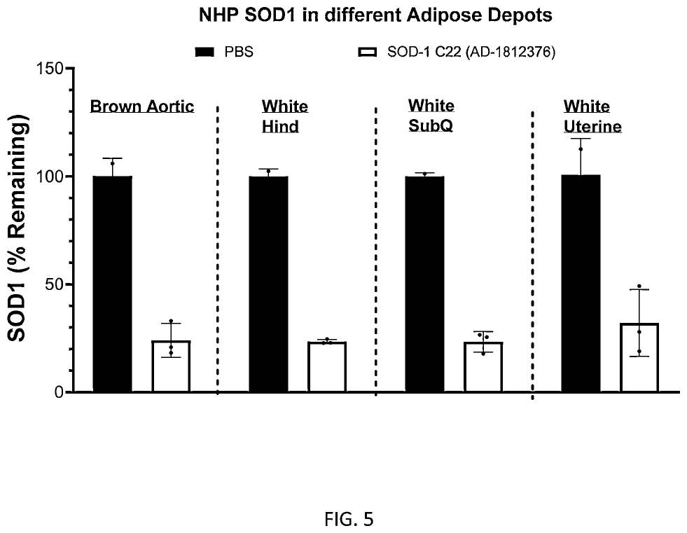

Changes in leptin levels were graphed in GraphPad Prism (Version 9.4.1 (681)) using the group average of serum leptin levels (FIGS. 6A, 7A, and 8A) or percent change in serum leptin (FIGS. 6B, 7B, and 8B) for each timepoint +/− standard error. Percent change was calculated with the serum concentration of leptin divided by the Day 0 Serum leptin concentration for each respective mouse. Individual serum leptin concentrations, group average leptin concentrations, and group leptin concentration standard error are listed in Table 14, Table 15, and Table 16, respectively. Individual percent change in serum leptin concentrations, group average percent change in serum leptin concentrations, and group standard error of leptin concentration are listed in Table 17, Table 18, and Table 19, respectively.

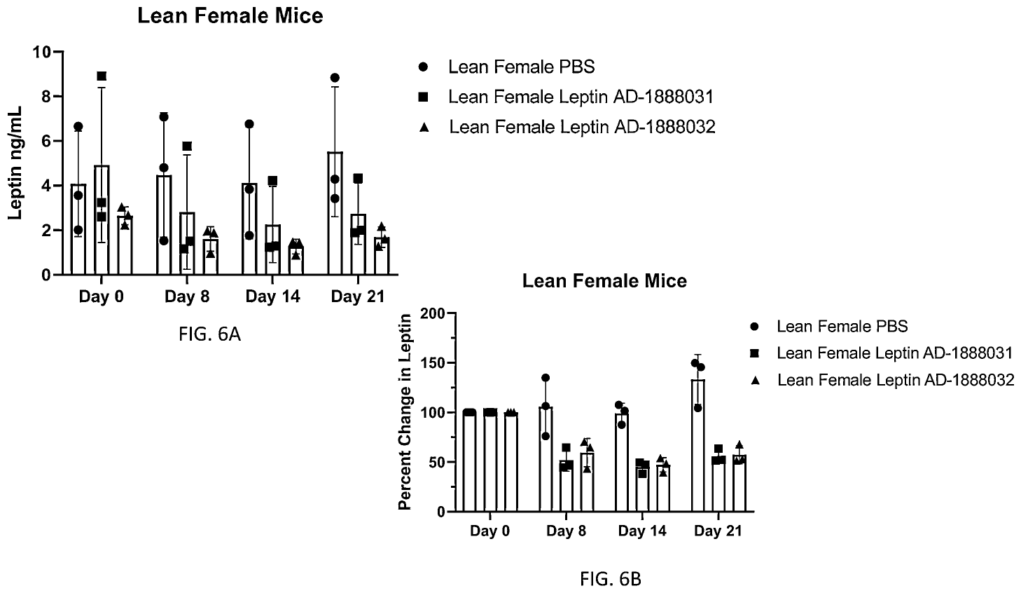

The data demonstrate that a dose of 5 mg/kg of AD-1888031.1 and AD-1888032.1, comprising a C22 hydrocarbon chain conjugated to the sense strand at position 6, counting from the 5′-end of the sense strand, was able to reduce circulating leptin by about 50% at two weeks in lean female mice (FIGS. 6A and 6B), and by about 65% at two weeks in lean male mice (FIGS. 7A and 7B). Both female and male mice showed minimal recovery of leptin expression at week 3.

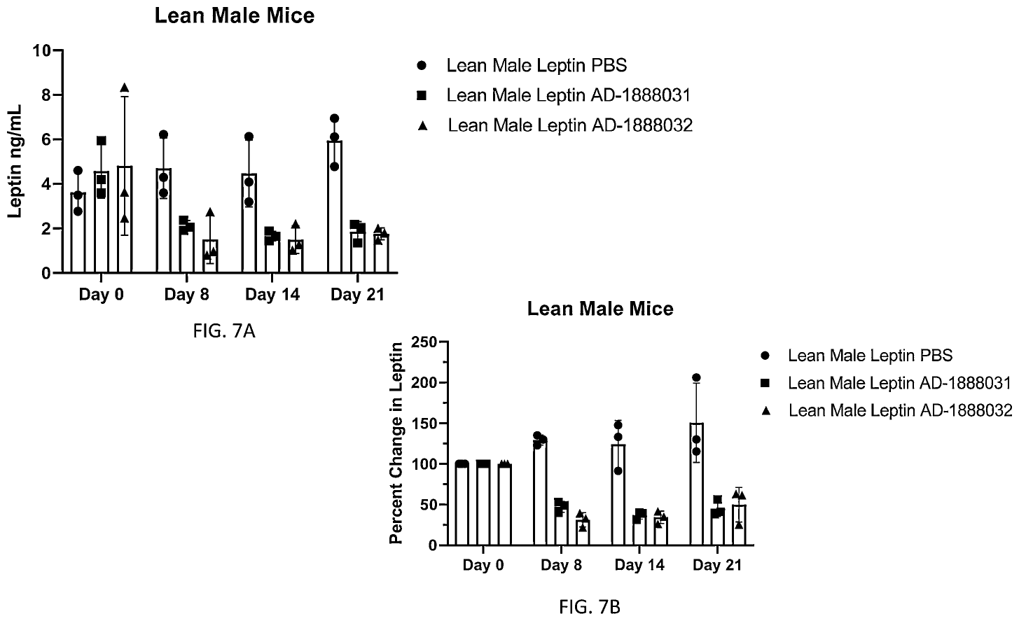

The effect of C22-conjugated dsRNA agents on male mice fed with high fat diet was also evaluated and shown in FIGS. 8A and 8B. A dose of 5 mg/kg of AD-1888031.1 and AD-1888032.1 dramatically reduced circulating leptin in high fat diet mice by about 90%, with only slight recovery of leptin expression at Day 21.

The levels of inhibition of the dsRNA agents increased from female mice, to male mice, to high fat diet male mice, suggesting that body weight may be a driving factor for gene silencing. For example, the heavier the mice (i.e., more body weight), the more dsRNA agents each animal would have received, and the more inhibition on leptin expression. In general, each lean female animal would have received about 0.11 mg dsRNA agent, while each lean male animal and each high fat diet male animal would have received about 0.14 mg and about 0.23 mg dsRNA agent, respectively.

In conclusion, the C22-conjugated dsRNA agents, e.g., AD-1888031.1 and AD-1888032.1, comprising a C22 hydrocarbon chain conjugated to the sense strand at position 6, counting from the 5′-end of the sense strand, were able to target leptin, an adipocyte specific gene, and potently and durably inhibit leptin expression levels in lean female mice, lean male mice, and male mice fed with a high fat diet. Moreover, a partial reduction in leptin levels may also prevent leptin resistance in mice and, thus, improve metabolism and reduce body weight.

TABLE 14
Individual Mouse Serum Concentrations
Serum Leptin (ng/mL)

| | | | Day | Day | Day |
|---|---|---|---|---|---|
| Treatment Group | Animal ID | Day 0 | 8 | 14 | 21 |
| Lean Female PBS | 1001 | 3.57 | 4.81 | 3.84 | 4.29 |
| | 1002 | 2.01 | 1.53 | 1.77 | 3.42 |
| | 1003 | 6.66 | 7.08 | 6.76 | 8.84 |
| Lean Female | 2001 | 3.24 | 1.50 | 1.24 | 2.00 |
| Leptin AD-1888031 | 2002 | 2.60 | 1.16 | 1.29 | 1.89 |
| | 2003 | 8.91 | 5.77 | 4.22 | 4.33 |
| Lean Female | 3001 | 3.03 | 1.96 | 1.47 | 2.18 |
| Leptin AD-1888032 | 3002 | 2.23 | 0.97 | 0.88 | 1.28 |
| | 3003 | 2.67 | 1.88 | 1.44 | 1.61 |
| Lean Male Leptin PBS | 4001 | 2.77 | 3.59 | 4.09 | 6.11 |
| | 4002 | 3.50 | 4.30 | 3.19 | 4.78 |
| | 4003 | 4.60 | 6.22 | 6.13 | 6.94 |
| Lean Male | 5001 | 5.94 | 2.37 | 1.88 | 2.18 |
| Leptin AD-1888031 | 5002 | 3.60 | 1.91 | 1.44 | 2.04 |
| | 5003 | 4.20 | 2.06 | 1.65 | 1.34 |
| Lean Male | 6001 | 2.46 | 0.96 | 1.02 | 1.47 |
| Leptin AD-1888032 | 6002 | 8.34 | 2.75 | 2.20 | 1.80 |
| | 6003 | 3.63 | 0.79 | 1.27 | 2.00 |
| HFD Male | 7001 | 91.58 | 3.05 | 4.04 | 7.05 |
| Leptin AD-1888031 | 7002 | 116.72 | 3.56 | 4.12 | 6.77 |
| | 7003 | 92.96 | 5.76 | 5.65 | 6.68 |
| HFD Male | 8001 | 81.84 | 2.94 | 3.54 | 4.40 |
| Leptin AD-1888032 | 8002 | 83.77 | 4.50 | 3.91 | 6.28 |
| | 8003 | 129.14 | 3.01 | 3.96 | 4.03 |

TABLE 15
Group Average Serum Leptin Concentrations
Group Average Serum Leptin (ng/mL)

| | | | Day | Day | Day |
|---|---|---|---|---|---|
| Treatment Group | Animal ID | Day 0 | 8 | 14 | 21 |
| Lean Female PBS | 1001 | 4.08 | 4.47 | 4.12 | 5.52 |
| | 1002 | | | | |
| | 1003 | | | | |
| Lean Female | 2001 | 4.92 | 2.81 | 2.25 | 2.74 |
| Leptin AD-1888031 | 2002 | | | | |
| | 2003 | | | | |
| Lean Female | 3001 | 2.65 | 1.60 | 1.26 | 1.69 |
| Leptin AD-1888032 | 3002 | | | | |
| | 3003 | | | | |
| Lean Male Leptin PBS | 4001 | 3.62 | 4.70 | 4.47 | 5.94 |
| | 4002 | | | | |
| | 4003 | | | | |
| Lean Male | 5001 | 4.58 | 2.11 | 1.66 | 1.85 |
| Leptin AD-1888031 | 5002 | | | | |
| | 5003 | | | | |
| Lean Male | 6001 | 4.81 | 1.50 | 1.50 | 1.76 |
| Leptin AD-1888032 | 6002 | | | | |
| | 6003 | | | | |
| HFD Male | 7001 | 100.42 | 4.12 | 4.60 | 6.83 |
| Leptin AD-1888031 | 7002 | | | | |
| | 7003 | | | | |
| HFD Male | 8001 | 98.25 | 3.49 | 3.80 | 4.90 |
| Leptin AD-1888032 | 8002 | | | | |
| | 8003 | | | | |

TABLE 16
Group Standard Error for Serum Leptin
Group Standard Error Serum Leptin (ng/mL)

| Treatment | Animal | | | | |
|---|---|---|---|---|---|
| Group | ID | Day 0 | Day 8 | Day 14 | Day 21 |
| Lean | 1001 | 2.363317 | 2.789439 | 2.508842 | 2.909304 |
| Female PBS | 1002 | | | | |
| | 1003 | | | | |
| Lean Female | 2001 | 3.474091 | 2.568692 | 1.707754 | 1.377894 |
| Leptin | 2002 | | | | |
| AD-1888031 | 2003 | | | | |
| Lean Female | 3001 | 0.40135 | 0.552431 | 0.329123 | 0.457212 |
| Leptin | 3002 | | | | |
| AD-1888032 | 3003 | | | | |
| Lean Male | 4001 | 0.924664 | 1.358248 | 1.50584 | 1.094218 |
| Leptin PBS | 4002 | | | | |
| | 4003 | | | | |
| Lean Male | 5001 | 1.219495 | 0.2369 | 0.215962 | 0.448158 |
| Leptin | 5002 | | | | |
| AD-1888031 | 5003 | | | | |
| Lean Male | 6001 | 3.116211 | 1.083027 | 0.620133 | 0.265945 |
| Leptin | 6002 | | | | |
| AD-1888032 | 6003 | | | | |
| HFD Male | 7001 | 14.12996 | 1.442328 | 0.906329 | 0.191559 |
| Leptin | 7002 | | | | |
| AD-1888031 | 7003 | | | | |
| HFD Male | 8001 | 26.76696 | 0.881409 | 0.231078 | 1.203625 |
| Leptin | 8002 | | | | |
| AD-1888032 | 8003 | | | | |

TABLE 17
Percent Change in Serum Leptin for each Individual Animal
Percent Change in Serum Leptin

| | | Day | | Day | Day |
|---|---|---|---|---|---|
| Treatment Group | Animal ID | 0 | Day 8 | 14 | 21 |
| Lean Female PBS | 1001 | 100 | 134.86 | 107.66 | 104.39 |
| | 1002 | 100 | 76.06 | 87.67 | 149.70 |
| | 1003 | 100 | 106.38 | 101.55 | 145.50 |
| Lean Female | 2001 | 100 | 46.47 | 38.31 | 51.48 |
| Leptin AD-1888031 | 2002 | 100 | 44.60 | 49.66 | 63.56 |
| | 2003 | 100 | 64.76 | 47.40 | 52.33 |
| Lean Female | 3001 | 100 | 64.70 | 48.36 | 67.88 |
| Leptin AD-1888032 | 3002 | 100 | 43.38 | 39.63 | 51.34 |
| | 3003 | 100 | 70.39 | 53.90 | 52.89 |
| Lean Male Leptin PBS | 4001 | 100 | 129.83 | 147.70 | 206.10 |
| | 4002 | 100 | 122.84 | 91.31 | 115.28 |
| | 4003 | 100 | 135.04 | 133.18 | 130.02 |
| Lean Male | 5001 | 100 | 39.95 | 31.56 | 38.11 |
| Leptin AD-1888031 | 5002 | 100 | 53.15 | 40.17 | 56.38 |
| | 5003 | 100 | 48.98 | 39.20 | 41.13 |
| Lean Male | 6001 | 100 | 39.24 | 41.63 | 63.01 |
| Leptin AD-1888032 | 6002 | 100 | 32.95 | 26.38 | 25.46 |
| | 6003 | 100 | 21.90 | 35.13 | 61.44 |
| HFD Male | 7001 | 100 | 3.07 | 5.15 | 7.69 |
| Leptin AD-1888031 | 7002 | 100 | 1.49 | 3.05 | 5.80 |
| | 7003 | 100 | 5.02 | 5.52 | 7.19 |
| HFD Male | 8001 | 100 | 3.59 | 4.96 | 5.38 |
| Leptin AD-1888032 | 8002 | 100 | 4.58 | 4.31 | 7.49 |
| | 8003 | 100 | 2.12 | 2.94 | 3.12 |

TABLE 18
Group Average Percent Change in Serum Leptin
Group Average Percent Change in Serum Leptin

| | | Day | | Day | Day |
|---|---|---|---|---|---|
| Treatment Group | Animal ID | 0 | Day 8 | 14 | 21 |
| Lean Female PBS | 1001 | 100 | 105.77 | 98.96 | 133.20 |
| | 1002 | | | | |
| | 1003 | | | | |
| Lean Female | 2001 | 100 | 51.94 | 45.12 | 55.79 |
| Leptin AD-1888031 | 2002 | | | | |
| | 2003 | | | | |
| Lean Female | 3001 | 100 | 59.49 | 47.30 | 57.37 |
| Leptin AD-1888032 | 3002 | | | | |
| | 3003 | | | | |
| Lean Male Leptin PBS | 4001 | 100 | 129.23 | 124.06 | 150.47 |
| | 4002 | | | | |
| | 4003 | | | | |
| Lean Male | 5001 | 100 | 47.36 | 36.98 | 45.20 |
| Leptin AD-1888031 | 5002 | | | | |
| | 5003 | | | | |
| Lean Male | 6001 | 100 | 31.36 | 34.38 | 49.97 |
| Leptin AD-1888032 | 6002 | | | | |
| | 6003 | | | | |
| HFD Male | 7001 | 100 | 3.19 | 4.57 | 6.89 |
| Leptin AD-1888031 | 7002 | | | | |
| | 7003 | | | | |
| HFD Male | 8001 | 100 | 3.43 | 4.07 | 5.33 |
| Leptin AD-1888032 | 8002 | | | | |
| | 8003 | | | | |

TABLE 19
Group Standard Error in Percent Change of Serum Leptin
Group Standard Error Percent Change in Serum Leptin

| | Animal | Day | | | |
|---|---|---|---|---|---|
| Treatment Group | ID | 0 | Day 8 | Day 14 | Day 21 |
| Lean Female PBS | 1001 | 0 | 29.40431 | 10.24501 | 25.03372 |
| | 1002 | | | | |
| | 1003 | | | | |
| Lean Female | 2001 | 0 | 11.13863 | 6.006439 | 6.74159 |
| Leptin AD-1888031 | 2002 | | | | |
| | 2003 | | | | |
| Lean Female | 3001 | 0 | 14.23971 | 7.195551 | 9.134996 |
| Leptin AD-1888032 | 3002 | | | | |
| | 3003 | | | | |
| Lean Male | 4001 | 0 | 6.122133 | 29.28125 | 48.74046 |
| Leptin PBS | 4002 | | | | |
| | 4003 | | | | |
| Lean Male | 5001 | 0 | 6.748633 | 4.713475 | 9.792716 |
| Leptin AD-1888031 | 5002 | | | | |
| | 5003 | | | | |
| Lean Male | 6001 | 0 | 8.775138 | 7.657442 | 21.24019 |
| Leptin AD-1888032 | 6002 | | | | |
| | 6003 | | | | |
| HFD Male | 7001 | 0 | 1.765579 | 1.328912 | 0.982239 |
| Leptin AD-1888031 | 7002 | | | | |
| | 7003 | | | | |
| HFD Male | 8001 | 0 | 1.234817 | 1.030463 | 2.186293 |
| Leptin AD-1888032 | 8002 | | | | |
| | 8003 | | | | |

### Example 10: In Vivo Dose Response and Duration of Intravenously Administered Lipid Conjugated dsRNA Agents Targeting Myostatin

The effect of dsRNA agents targeting myostatin (MSTN1) on the mRNA levels of MTSN1 in skeletal and cardiac muscle was examined. The design of this study is shown in Table 20.

Briefly, female C57BL/6 mice, 6-8 weeks of age (N=3 per group), were intravenously administered a single 1 mg/kg, 2.5 mg/kg or 5 mg/kg dose of a dsRNA agent targeting MSTN1. The modified nucleotide sequences of the sense and antisense strands of the dsRNA agents are provided in Table 21.

On Day 14 or Day 56 post-dose, quadriceps, heart, lungs, liver and other tissues were collected, immediately flash-frozen in liquid nitrogen and stored at −80° C. Tissues were ground with a tissue grinder (Geno/Grinder 2010 SPEX Sample Prep) at 1250 rpm for 1.5 minute. RNA extraction and purification were conducted per protocols as described in Qiagen's RNeasy 96 Universal Tissue Handbook. Resulting RNA (1500 ng) was used for cDNA synthesis with the Applied Biosystems™ High-Capacity cDNA Reverse Transcription Kit. Levels of target mRNA were quantified by RT-qPCR using a Roche LightCycler® 480. Relative change in target mRNA expression was determined with ΔΔCt method by doubly normalizing to GAPDH and the average of controls.

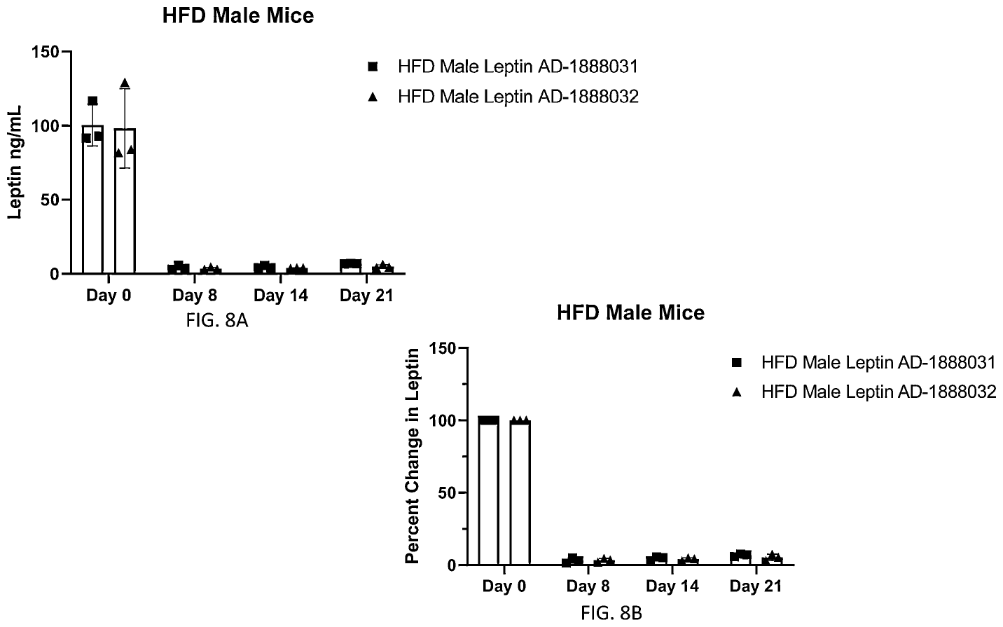

The results, shown in FIGS. 9A-9B, demonstrate that dsRNA agents comprising one or more lipophilic moieties (e.g., any compound or chemical moiety having an affinity for lipids, e.g., a C₂₂ hydrocarbon chain) conjugated at various positions on the sense or antisense strand inhibit MSTN1 in muscle tissue.

TABLE 20
Study design

| Group | siRNA | N | I.V. Dose | | sac | Collection |
|---|---|---|---|---|---|---|
| 1 | PBS | 3 | NA | | D 14 | Quad, heart |
| 2 | AD-1640773 | | 5 | mg/kg | | |
| 3 | AD-1684752 | | | | | |
| 4 | AD-1684750 | | | | | |
| 5 | AD-1684751 | | | | | |
| 6 | AD-1640773 | | 2.5 | mg/kg | | |
| 7 | AD-1684750 | | | | | |
| 8 | AD-1684751 | | | | | |
| 9 | AD-1640773 | | 1 | mg/kg | | |
| 10 | AD-1684750 | | | | | |
| 11 | AD-1684751 | | | | | |
| 12 | PBS | | NA | | D 56 | Quad, heart |
| 13 | AD-68685 | | 5 | mg/kg | | |
| 14 | AD-1640773 | | | | | |
| 15 | AD-1640773 | | 2.5 | mg/kg | | |
| 16 | AD-1640773 | | 1 | mg/kg | | |

TABLE 21

| | | | | | Molecular |
|---|---|---|---|---|---|
| | | | | Molecular | Weight |
| Duplex ID | Strand | Target | Sequence 5′ to 3′ | Weight | Found |
| AD-1640773 | sense | MSTN | asusggc(Ada)AfaGfAfAfc | 7387.441 | 7383.514 |
| | | | aaauaausasa | | |
| | antis | MSTN | VPusUfsauuAfuUfUfguucU | 7538.806 | 7533.956 |
| | | | fuUfgccaususa | | |
| AD-1684752 | sense | MSTN | asusggc(Ada)AfaGfAfAfc | 7387.441 | 7383.514 |
| | | | aaauaausasa | | |
| | antis | MSTN | usUfsauuAfuUfUfguucUfu | 7461.807 | 7457.976 |
| | | | Ufgccaususa | | |
| AD-1684750 | sense | MSTN | asusggc(Ada)AfaGfAfAfc | 7387.441 | 7383.514 |
| | | | aaauaausasa | | |
| | antis | MSTN | (Pmmds)usUfsauuAfuUfUf | 7704.117 | 7699.942 |
| | | | guucUfuUfgccaususa | | |
| AD-1684751 | sense | MSTN | asusggc(Ada)AfaGfAfAfc | 7387.441 | 7383.514 |
| | | | aaauaausasa | | |
| | antis | MSTN | (tPmmds)usUfsauuAfuUfU | 7704.113 | 7699.942 |
| | | | fguucUfuUfgccaususa | | |
| AD-68685 | sense | MSTN | asusggcaAfaGfAfAfcaaau | 7765.662 | 7761.684 |
| | | | aauaaL10 | | |
| | antis | MSTN | VPusUfsauuAfuUfUfguucU | 7538.806 | 7533.956 |
| | | | fuUfgccaususa | | |

### Example 11: In Vivo Effect of Intravenously Administered Lipid Conjugated dsRNA Agents Targeting SOD1

The effect of dsRNA agents targeting SOD1 and comprising one or more C₂₂ hydrocarbon chains conjugated to position 6 on the sense strand, counting from the 5′-end of the sense strand, were examined for knockdown in muscle tissues.

The design of this study is shown in Table 22.

Briefly, female C57BL/6 mice, 6-8 weeks of age (N=3 per group), were intravenously administered a single 2 mg/kg dose of AD-1427062, a dsRNA agent targeting SOD1. The modified nucleotide sequences of the sense and antisense strands of AD-1427062 are provided in Table 23.

On Day 14 post-dose, quadriceps, heart, lungs, liver, and other tissues were collected, immediately flash-frozen in liquid nitrogen and stored at −80° C. Tissues were ground with a tissue grinder (Geno/Grinder 2010 SPEX Sample Prep) at 1250 rpm for 1.5 minute. RNA extraction and purification were conducted per protocols as described in Qiagen's RNeasy 96 Universal Tissue Handbook. Resulting RNA (1500 ng) was used for cDNA synthesis with the Applied Biosystems™ High-Capacity cDNA Reverse Transcription Kit. Levels of target mRNA were quantified by RT-qPCR using a Roche LightCycler® 480. Relative change in target mRNA expression was determined with ΔΔCt method by doubly normalizing to GAPDH and the average of controls.

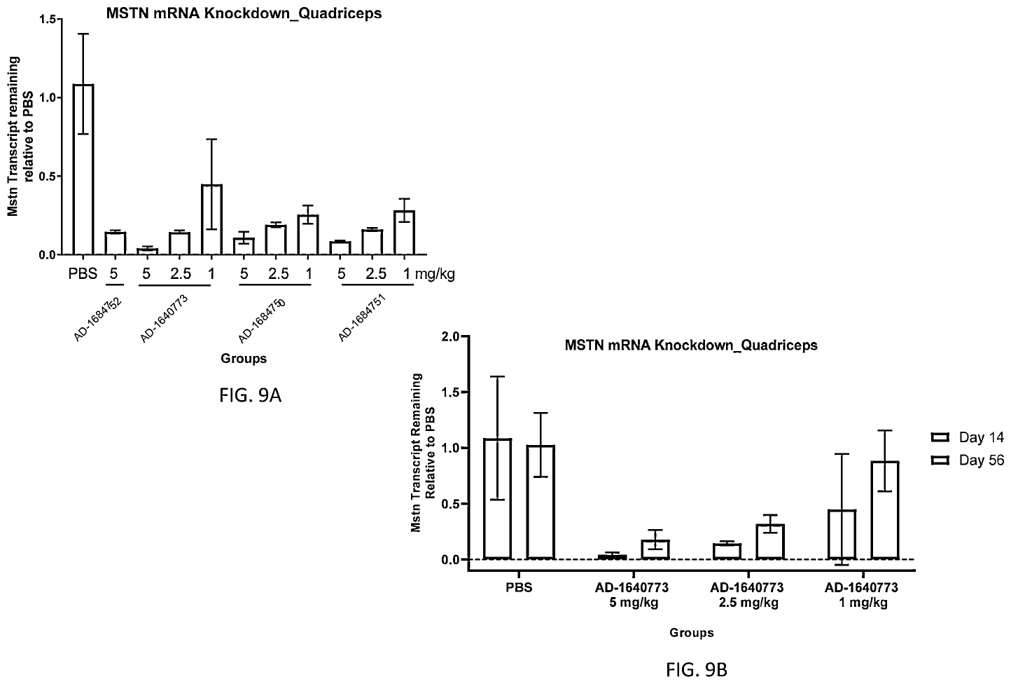

The results, shown in FIG. 10, demonstrate that administration of a single dose of a dsRNA agent targeting SOD1 and comprising one or more C₂₂ hydrocarbon chains conjugated to position 6 on the sense strand, counting from the 5′-end of the sense strand, potently knocks down SOD1 expression in muscle tissues.

TABLE 22
Study Design

| Group | siRNA | N | I.V. Dose | sac | Collection |
|---|---|---|---|---|---|
| 1 | PBS | 3 | NA | D14 | Left quad, right quad, |
| 2 | AD-1427062 | | 2 mg/kg | | diaphragm, |
| | | | | | gastrocnemius |

TABLE 23

| | | | | | Molecular |
|---|---|---|---|---|---|
| Duplex | | | | Molecular | Weight |
| ID | Strand | Target | Sequence 5′ to 3′ | Weight | Found |
| AD-1427062 | sense | SOD1 | csasuuu(Uda)AfaUfCf | 7128.132 | 7124.348 |
| | | | Cfucacucuasasa | | |
| | antis | SOD1 | VPusUfsuagAfgUfGfag | 7851.156 | 7847.154 |
| | | | gaUfuAfaaaugsasg | | |

### Example 12: In Vivo Multidose Comparison of Intravenously or Subcutaneously Administered Lipid Conjugated dsRNA Agents Targeting SOD1

The effect of route of administration of lipid conjugated dsRNA agents targeting SOD1, e.g., dsRNA agents targeting SOD1 and comprising one or more C₂₂ hydrocarbon chains conjugated to position 6 on the sense strand, counting from the 5′-end of the sense strand) on the mRNA levels of SOD1 in skeletal and cardiac muscle was examined. The design of this study is shown in Table 24.

Briefly, female C57BL/6 mice, 6-8 weeks of age (N=3 per group), were intravenously or subcutaneously administered a single 2 mg/kg dose or a 1 mg/kg dose on Day 0 followed by a second 1 mg/kg dose 1 week later (1 mg/kg dose X2) of AD-1812376, a dsRNA agent targeting SOD1. The modified nucleotide sequences of the sense and antisense strands of AD-1812376 are provided in Table 25.

On Day 28 post-dose, quadriceps, heart, lungs, liver, and other tissues were collected, immediately flash-frozen in liquid nitrogen and stored at −80° C. Tissues were ground with a tissue grinder (Geno/Grinder 2010 SPEX Sample Prep) at 1250 rpm for 1.5 minute. RNA extraction and purification were conducted per protocols as described in Qiagen's RNeasy 96 Universal Tissue Handbook. Resulting RNA (1500 ng) was used for cDNA synthesis with the Applied Biosystems™ High-Capacity cDNA Reverse Transcription Kit. Levels of target mRNA were quantified by RT-qPCR using a Roche LightCycler® 480. Relative change in target mRNA expression was determined with ΔΔCt method by doubly normalizing to GAPDH and the average of controls.

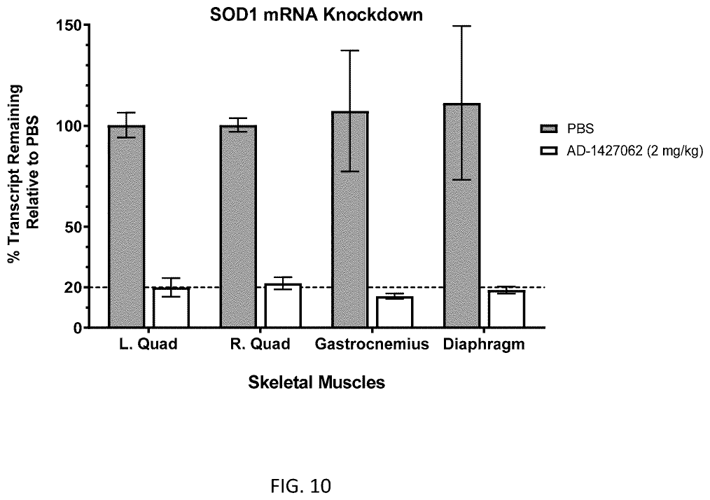

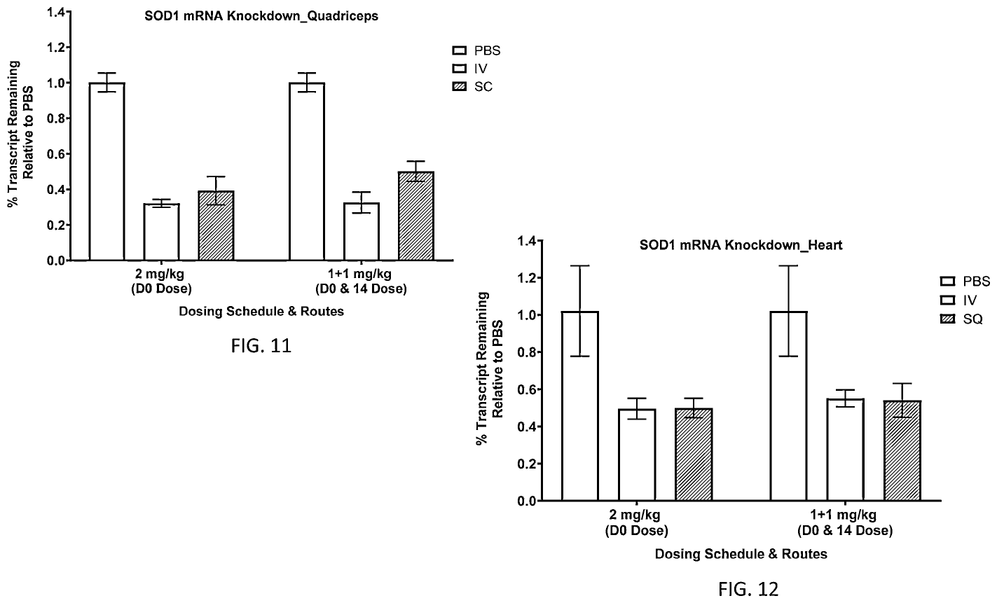

The result, shown in FIGS. 11 and 12, demonstrate that dsRNA agents targeting SOD1 and comprising one or more C₂₂ hydrocarbon chains conjugated to position 6 on the sense strand, counting from the 5′-end of the sense strand, knocks down SOD1 expression in muscle tissues irrespective of intravenous or subcutaneous administration.

TABLE 24
Study design

| Group | siRNA | Route of Admin. | Dose | | sac | N | Collection |
|---|---|---|---|---|---|---|---|
| 1 | PBS | IV | NA | | D 21 | 3 | Quad, heart, |
| 2 | AD-1812376 | IV | 2 | mg/kg | D 28 | | adipose |
| 3 | AD-1812376 | Subcutaneous | 2 | mg/kg | | | |
| 4 | AD-1812376 | IV | 1 + 1 | mg/kg | | | |
| 5 | AD-1812376 | Subcutaneous | 1 + 1 | mg/kg | | | |

TABLE 25

| | | | | | Molecular |
|---|---|---|---|---|---|
| | | | | Molecular | Weight |
| Duplex ID | Strand | Target | Sequence 5′ to 3′ | Weight | Found |
| AD-1812376 | sense | SOD1 | ususggg(Cda)AfaAfGfG | 7468.423 | 7464.473 |
| | | | fuggaaaugsasa | | |
| | antis | SOD1 | VPusUfscauUfuCfCfacc | 7532.881 | 7529.035 |
| | | | uUfuGfcccaasgsu | | |

### Example 13: In Vivo Dose Response of Intravenously Administered Lipid Conjugated siRNA Agent Targeting Myostatin in Monkeys

The effect of dsRNA agents targeting Myostatin (MTSN) and comprising one or more C₂₂ hydrocarbon chains conjugated to position 6 on the sense strand, counting from the 5′-end of the sense strand, were examined for knockdown in muscle tissues.

The modified nucleotide sequences of the duplexes used in this study are provided in Table 26.

The effect of the agents in Table 26 was examined in vivo in non-human primates as summarized in Table 27. Briefly, animals were intravenously administered a single 2 mg/kg or 5 mg/kg dose of the agent on Day 0. On Day 56, animals were sacrificed and heart, gastrocnemius and quadriceps tissues were collected.

TABLE 26

| | | | | | Molecular |
|---|---|---|---|---|---|
| Duplex | | | | Molecular | Weight |
| ID | Strand | Target | Sequence 5′ to 3′ | Weight | Found |
| AD-1640773 | sense | MSTN | asusggc(Ada)AfaGfAf | 7387.441 | 7383.514 |
| | | | Afcaaauaausasa | | |
| | antis | MSTN | VPusUfsauuAfuUfUfgu | 7538.806 | 7533.956 |
| | | | ucUfuUfgccaususa | | |

TABLE 27
Study Design

| Group | siRNA | N | I.V. Dose | sac | Collection |
|---|---|---|---|---|---|
| 1 | PBS | 2 | NA | D56 | Quad, |
| 2 | AD-1640773 | 3 | 2 mg/kg | | gastrocnemius |
| 3 | AD-1640773 | 3 | 5 mg/kg | | |

For quantification of MTSN protein concentration, tissues were pulverized into powder and samples lysates were prepared in mass to volume ratio of 1:20, and clarified lysate was used for Myostatin mRNA and total protein analysis. Tissue lysates were analyzed for MTSN protein using a commercially available sandwich ELISA per the manufacturer instructions (R&D Systems, DGDF80). In brief, standards and samples were added to microplates pre-coated with a monoclonal antibody specific for mature MTSN. Mature MTSN present in samples and standards were immobilized onto microplates. Plates were washed to remove unbound material, followed by addition of horseradish peroxidase-conjugated monoclonal antibody specific for mature MTSN. Plates were washed to remove any unbound antibody-enzyme reagent, and a substrate solution was added to each well. The substrate reacts with immobilized antibody-enzyme complex to generate a color product proportional to the amount of MTSN present in the unknown samples or standards. The color development is stopped and the intensity of the color is measured. For tissue, the mean concentration of GDF-8 protein (pg/mL) was adjusted to total protein concentration (mg/mL), and values were reported in pg/mg. The mean tissue MTSN concentration for each animal was normalized to the mean GDF-8 concentration of control animals to determine the fraction of myostatin protein relative to control.

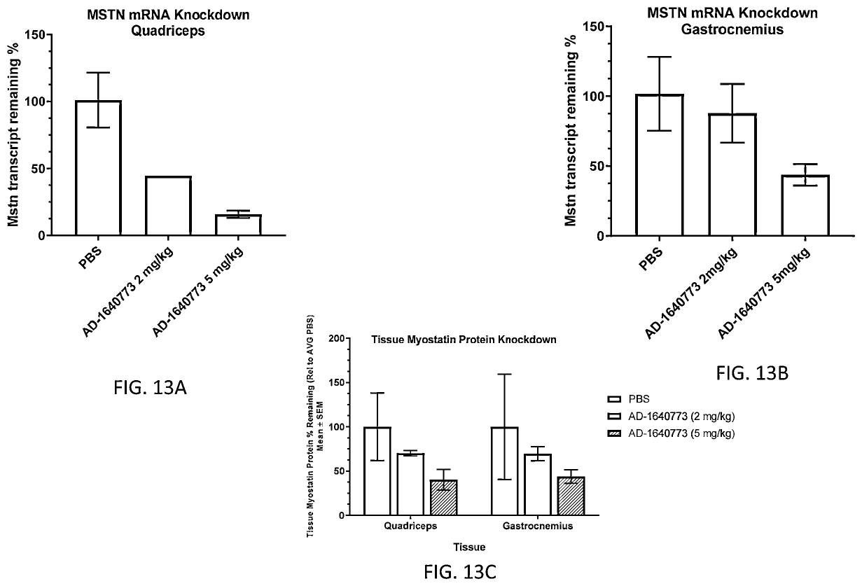

The results of administration of a single 5 mg/kg dose of AD-1640773, a dsRNA agents targeting Myostatin (MTSN) and comprising one or more C₂₂ hydrocarbon chains conjugated to position 6 on the sense strand, counting from the 5′-end of the sense strand, on MTSN protein concentrations is provided FIG. 13C and demonstrates that intravenous administration of the dsRNA agent knocksdown MTSN protein in muscle tissue.

For quantification of MTSN mRNA knockdown, heart, quadriceps, and gastrocnemius tissues were analyzed for myostatin (MSTN) mRNA using an exploratory reverse transcription quantitative polymerase chain reaction (RT-qPCR) method as outlined below.

Frozen heart, quadriceps, and gastrocnemius tissue samples were pulverized using a Genogrinder, further homogenized with a TissueLyser LT for 2 minutes with 50 cycles/sec before being lysed in 350 μL Buffer MR1 plus 6 μL reducing agent TCEP from the MACHEREY-NAGEL NucleoMag® RNA kit. Lysates were cleared by centrifugation at 5600×g for 5 minutes in atabletop centrifuge. RNA was isolated from cleared lysates using the NucleoMag® RNA kit according to the manufacturer's protocol. RNA concentration and integrity were measured using the RNA ScreenTape Analysis on an Agilent TapeStation instrument. All RNA samples were diluted to 7.14 ng/μL in nuclease-free water, from which 14 μL, or 100 ng, was used for complementary DNA (cDNA) synthesis in a reverse transcription (RT) reaction using the Invitrogen™ SuperScript™ IV VILO™ Master Mix in a total reaction volume of 20 μL. For quantitative polymerase chain reaction (qPCR) reactions, 2 μL, containing 10 ng of cDNA, was used in a total reaction volume of 20 μL. qPCR was performed using the Applied Biosystems™ TaqMan® Fast Advanced Master Mix in the Applied Biosystems™ ViiA7 Real-Time PCR System. Commercially available, Macaca fascicularis-specific MSTN, peptidyl-prolyl cis-trans isomerase B (PPIB), and glyceraldehyde-3-phosphate dehydrogenase (GAPDH) TaqMan® primer/probes were used for qPCR analysis. Each sample was analyzed in duplicate by qPCR, with each reaction a duplex of fluorescein amidite (FAM)-labeled MSTN plus VIC (Aequorea Victoria Green Fluorescent Protein)-labeled GAPDH or PPIB. Data were acquired using ThermoFisher QuantStudio v1.3 software with 40 cycles and data acquisition at the end of each cycle.

Cycle threshold (Ct) values were determined using the ThermoFisher QuantStudio v1.3 software. The cycle threshold was set to 0.2 for the 40-cycle qPCR assay. The Delta-Delta Threshold Cycle (Relative Quantification) [ΔΔCt (RQ)] method was used to calculate fold-change in MSTN mRNA between control and treated samples using Microsoft® Office Excel 2016 (Microsoft Corporation [Redmond, WA]). First, the difference between MSTN and each housekeeping gene was calculated by subtracting the average housekeeping gene Ct from the average MSTN Ct for each duplex qPCR reaction. The difference between the delta Ct of each sample and the delta Ct of the average delta Ct of the control group (vehicle treated) was used to calculate the fold-change in MSTN mRNA. The fold-change in MSTN mRNA from all housekeeping genes was then averaged using a geometric mean and was reported as the “% mRNA remaining”, where the control group “% mRNA” remaining was set to 100% remaining.

The results of administration of single 2 mg/kg or 5 mg/kg doses of AD-1640773, a dsRNA agents targeting Myostatin (MTSN) and comprising one or more C₂₂ hydrocarbon chains conjugated to position 6 on the sense strand, counting from the 5′-end of the sense strand, on MTSN mRNA knockdown are provided FIGS. 13A and B and demonstrates that intravenous administration of the dsRNA agent knocks down MTSN mRNA in muscle tissue.

### Example 14: dsRNA Synthesis

siRNAs targeting the human leptin (LEP) gene (human: GenBank NM_000230.3, NCBI GeneID: 3952) were designed using custom R and Python scripts. The human LEP REFSEQ NM_000230.3 mRNA has a length of 3427 bases. siRNAs targeting the mouse leptin (Lep) gene (mouse: GenBank NM_008493.3, NCBI GeneID: 16846 were designed using custom R and Python scripts. The mouse Lep REFSEQ NM_008493.3 mRNA has a length of 3257 bases.

siRNAs comprising a GalNAc conjugate targeting ligand were designed and synthesized as described above. siRNAs comprising an unsaturated C₂₂ hydrocarbon chain conjugated to the sense strand at position 6, counting from the 5′-end of the sense strand, were designed and synthesized as described above.

Detailed lists of the modified LEP sense and antisense strand nucleotide sequences comprising an unsaturated C₂₂ hydrocarbon chain conjugated to the sense strand at position 6, counting from the 5′-end of the sense strand, are shown in Tables 29 and 35, and the corresponding unmodified LEP sense and antisense nucleotide sequences are shown in Tables 28 and 34.

Detailed lists of the modified LEP sense and antisense strand nucleotide sequences comprising a GalNAc conjugate targeting ligand are shown in Tables 31 and 33, and the corresponding unmodified LEP sense and antisense nucleotide sequences are shown in Table 30 and 32.

It is to be understood that, throughout the application, a duplex name without a decimal is equivalent to a duplex name with a decimal which merely references the batch number of the duplex. For example, AD-959917 is equivalent to AD-959917.1.

TABLE 28
Unmodified Sense and Antisense Strand Sequences of LEP dsRNA Agents

| | | SEQ | NM_000230.3 | | | SEQ | NM_000230.3 | |
|---|---|---|---|---|---|---|---|---|
| Duplex | Sense Strand | ID | Start | End | Antisense Strand | ID | Start | End |
| Name | Sequence 5′ to 3′ | NO: | Position | Position | Sequence 5′ to 3′ | NO: | Position | Position |
| AD-1949134 | AAUGUGUUUUCUGAAUAACAA | | 1436 | 1456 | UUGUUAUUCAGAAAACACAUUCU | | 1434 | 1456 |
| AD-1949135 | UGAAUCAAAGCAGUUAACUUA | | 2158 | 2178 | UAAGUUAACUGCUUUGAUUCAAG | | 2156 | 2178 |
| AD-1949136 | GUGAAACUUCCUAAGUAUAAA | | 3024 | 3044 | UUUAUACUUAGGAAGUUUCACAC | | 3022 | 3044 |
| AD-1949137 | UCUCGUAACUGGUUUCAUUUA | | 1255 | 1275 | UAAAUGAAACCAGUUACGAGAGA | | 1253 | 1275 |
| AD-1949138 | UGCACUUUGUAACAUGUUUAA | | 2433 | 2453 | UUAAACAUGUUACAAAGUGCAAG | | 2431 | 2453 |
| AD-1949139 | CUGUCCAAGGAAACUUGAAUA | | 2143 | 2163 | UAUUCAAGUUUCCUUGGACAGAU | | 2141 | 2163 |
| AD-1949140 | UGAAGAAAACUCCUUUAGCAA | | 1951 | 1971 | UUGCUAAAGGAGUUUUCUUCACC | | 1949 | 1971 |
| AD-1949141 | GGAGUGAGAUCAUUUUCUUAA | | 1479 | 1499 | UUAAGAAAAUGAUCUCACUCCUU | | 1477 | 1499 |
| AD-1949142 | UUGUCUGUUUUUGUAACUUAA | | 3048 | 3068 | UUAAGUUACAAAAACAGACAACC | | 3046 | 3068 |
| AD-1949143 | GCAUGCCAAAUUGUAGUUCUA | | 1864 | 1884 | UAGAACUACAAUUUGGCAUGCAA | | 1862 | 1884 |
| AD-1949144 | AAGGGUAAAGAAGUUUGAUAA | | 2839 | 2859 | UUAUCAAACUUCUUUACCCUUCA | | 2837 | 2859 |
| AD-1949145 | UUAGGAUGUUUUUCAUGAAAA | | 1513 | 1533 | UUUUCAUGAAAAACAUCCUAAGC | | 1511 | 1533 |
| AD-1949146 | CCUCAUCAAGACAAUUGUCAA | | 156 | 176 | UUGACAAUUGUCUUGAUGAGGGU | | 154 | 176 |
| AD-1949147 | UCACAUUCAGGAAGAUGAAAA | | 2057 | 2077 | UUUUCAUCUUCCUGAAUGUGACC | | 2055 | 2077 |
| AD-1949148 | UUUGUCAAGUGUCAUAUGUAA | | 1363 | 1383 | UUACAUAUGACACUUGACAAAAC | | 1361 | 1383 |
| AD-1949149 | CCUUUGAAACAAGAUAACUGA | | 2490 | 2510 | UCAGUUAUCUUGUUUCAAAGGGA | | 2488 | 2510 |
| AD-1949150 | CUCCUUUUGCUUGAAACCAAA | | 727 | 747 | UUUGGUUUCAAGCAAAAGGAGGC | | 725 | 747 |
| AD-1949151 | CACCAGGAUCAAUGACAUUUA | | 174 | 194 | UAAAUGUCAUUGAUCCUGGUGAC | | 172 | 194 |
| AD-1949152 | CUUCAAAUCCAUCCAGAAUAA | | 1634 | 1654 | UUAUUCUGGAUGGAUUUGAAGCA | | 1632 | 1654 |
| AD-1949153 | GGGCUGAAAGCCAUUUGUUGA | | 1663 | 1683 | UCAACAAAUGGCUUUCAGCCCUU | | 1661 | 1683 |
| AD-1949154 | GAGCUGAACCCAUUUUGAGUA | | 1176 | 1196 | UACUCAAAAUGGGUUCAGCUCAG | | 1174 | 1196 |
| AD-1949155 | GCUGUGUUUUUGCUAUCACAA | | 2108 | 2128 | UUGUGAUAGCAAAAACACAGCAG | | 2106 | 2128 |
| AD-1949156 | AUCCAGGACUCUGUCAAUUUA | | 661 | 681 | UAAAUUGACAGAGUCCUGGAUAA | | 659 | 681 |
| AD-1949157 | UUCCAGAAACGUGAUCCAAAA | | 327 | 347 | UUUUGGAUCACGUUUCUGGAAGG | | 325 | 347 |
| AD-1949158 | CAUGGCAGUGUUCCUAUUUGA | | 1838 | 1858 | UCAAAUAGGAACACUGCCAUGUA | | 1836 | 1858 |
| AD-1949159 | AAGCCUUUUGCUCACAAAACA | | 2978 | 2998 | UGUUUUGUGAGCAAAAGGCUUAA | | 2976 | 2998 |
| AD-1949160 | CUACUGUGACUGAUGUUACAA | | 1275 | 1295 | UUGUAACAUCAGUCACAGUAGAA | | 1273 | 1295 |
| AD-1949161 | UGGAAGCACAUGUUUAUUUAA | | 1035 | 1055 | UUAAAUAAACAUGUGCUUCCAGC | | 1033 | 1055 |
| AD-1949162 | GCAUCUUAGCUUCUAUUAUAA | | 2462 | 2482 | UUAUAAUAGAAGCUAAGAUGCCC | | 2460 | 2482 |
| AD-1949163 | UUCAGCCAUCAACAAGAGUUA | | 832 | 852 | UAACUCUUGUUGAUGGCUGAAGA | | 830 | 852 |
| AD-1949164 | AGCUCACCCAAUAAACAUUAA | | 2232 | 2252 | UUAAUGUUUAUUGGGUGAGCUUC | | 2230 | 2252 |
| AD-1949165 | UGGCCCUAUCUUUUCUAUGUA | | 94 | 114 | UACAUAGAAAAGAUAGGGCCAAA | | 92 | 114 |
| AD-1949166 | GAUCACACUCUGGGUUUAUUA | | 1817 | 1837 | UAAUAAACCCAGAGUGUGAUCAA | | 1815 | 1837 |
| AD-1949167 | UCACCGGUUUGGACUUCAUUA | | 227 | 247 | UAAUGAAGUCCAAACCGGUGACU | | 225 | 247 |
| AD-1949168 | GAGCGAGAUUCCGUCUUAAAA | | 3349 | 3369 | UUUUAAGACGGAAUCUCGCUCUG | | 3347 | 3369 |
| AD-1949169 | UUAUCUUCUGCAAUUGCUUAA | | 1496 | 1516 | UUAAGCAAUUGCAGAAGAUAAGA | | 1494 | 1516 |
| AD-1949170 | GAGUAGCAAGUUCUAGAGAAA | | 2872 | 2892 | UUUCUCUAGAACUUGCUACUCUU | | 2870 | 2892 |
| AD-1949171 | CCUGUUUUGUUGGAAGGUUUA | | 2719 | 2739 | UAAACCUUCCAACAAAACAGGCU | | 2717 | 2739 |
| AD-1949172 | ACAGGAGAAUCGCUUAAACCA | | 3271 | 3291 | UGGUUUAAGCGAUUCUCCUGUCU | | 3269 | 3291 |
| AD-1949173 | ACUCUUCCAAAGGCAUAAGAA | | 699 | 719 | UUCUUAUGCCUUUGGAAGAGUGG | | 697 | 719 |
| AD-1949174 | GAUGGAUUUGAAGCAAAGCAA | | 1072 | 1092 | UUGCUUUGCUUCAAAUCCAUCCA | | 1070 | 1092 |
| AD-1949175 | CUCCUCCAAACAGAAAGUCAA | | 210 | 230 | UUGACUUUCUGUUUGGAGGAGAC | | 208 | 230 |
| AD-1949176 | CUGGAAAAGAGGAGUUUCGAA | | 2651 | 2671 | UUCGAAACUCCUCUUUUCCAGAA | | 2649 | 2671 |
| AD-1949177 | CAGUUUCCAAUCCCAUAGAUA | | 1146 | 1166 | UAUCUAUGGGAUUGGAAACUGCA | | 1144 | 1166 |
| AD-1949178 | ACCUGGCACAAUGGCUAAUUA | | 2996 | 3016 | UAAUUAGCCAUUGUGCCAGGUUU | | 2994 | 3016 |
| AD-1949179 | UCCAAGGACCAGGUUAUUUUA | | 1331 | 1351 | UAAAAUAACCUGGUCCUUGGAGA | | 1329 | 1351 |
| AD-1949180 | CCUGGGAAGGAAAAUGCAUUA | | 45 | 65 | UAAUGCAUUUUCCUUCCCAGGAU | | 43 | 65 |
| AD-1949181 | AGUCCAAGAUGACACCAAAAA | | 135 | 155 | UUUUUGGUGUCAUCUUGGACUUU | | 133 | 155 |
| AD-1949182 | UCCUCACAACCACCUAAUCAA | | 2944 | 2964 | UUGAUUAGGUGGUUGUGAGGAUC | | 2942 | 2964 |
| AD-1949183 | AGCAAGGCCAAAAUUACCAAA | | 1903 | 1923 | UUUGGUAAUUUUGGCCUUGCUUG | | 1901 | 1923 |
| AD-1949184 | CACUGAAUGCCUCAAUGUGAA | | 882 | 902 | UUCACAUUGAGGCAUUCAGUGAG | | 880 | 902 |
| AD-1949185 | UUACAUCACAGUGUUUGCAAA | | 1290 | 1310 | UUUGCAAACACUGUGAUGUAACA | | 1288 | 1310 |
| AD-1949186 | UCCAAGGAGUUCCAUGAAGAA | | 977 | 997 | UUCUUCAUGGAACUCCUUGGAAU | | 975 | 997 |
| AD-1949187 | AGGAUUGAAGAGCAUUGCAUA | | 630 | 650 | UAUGCAAUGCUCUUCAAUCCUGG | | 628 | 650 |
| AD-1949188 | AAGGGAGAAGGAUCUAGAAUA | | 1419 | 1439 | UAUUCUAGAUCCUUCUCCCUUCU | | 1417 | 1439 |
| AD-1949189 | UAUUAAAGGAGUUAAGAGUAA | | 2857 | 2877 | UUACUCUUAACUCCUUUAAUAUC | | 2855 | 2877 |
| AD-1949190 | GUGGUCUUUCCUAUCAUGGAA | | 1722 | 1742 | UUCCAUGAUAGGAAAGACCACUC | | 1720 | 1742 |
| AD-1949191 | UCUGCAUUUUAUUCUGGAUGA | | 1056 | 1076 | UCAUCCAGAAUAAAAUGCAGAAU | | 1054 | 1076 |
| AD-1949192 | UCCUGCAAGGACUACGUUAAA | | 580 | 600 | UUUAACGUAGUCCUUGCAGGAAG | | 578 | 600 |
| AD-1949193 | GUUCCCUCUGAGAAUUCCAAA | | 962 | 982 | UUUGGAAUUCUCAGAGGGAACCU | | 960 | 982 |
| AD-1949194 | GAAGCUGAUGCUUUGCUUCAA | | 1619 | 1639 | UUGAAGCAAAGCAUCAGCUUCUC | | 1617 | 1639 |
| AD-1949195 | AAAUACAUAAGACCAUAACAA | | 2527 | 2547 | UUGUUAUGGUCUUAUGUAUUUUC | | 2525 | 2547 |
| AD-1949196 | CCUUUGGAUGACCAGAACAAA | | 941 | 961 | UUUGUUCUGGUCAUCCAAAGGCU | | 939 | 961 |
| AD-1949197 | GGAGAUGCAGAGGUAAAAGUA | | 2746 | 2766 | UACUUUUACCUCUGCAUCUCCAC | | 2744 | 2766 |
| AD-1949198 | UUGGGUUCAUCUGAGCAAGAA | | 1206 | 1226 | UUCUUGCUCAGAUGAACCCAACC | | 1204 | 1226 |
| AD-1949199 | GCUCUCCAGUUAGUUCUCUCA | | 1239 | 1259 | UGAGAGAACUAACUGGAGAGCCA | | 1237 | 1259 |
| AD-1949200 | AGUGUGAGCAGUGAGUUACAA | | 2763 | 2783 | UUGUAACUCACUGCUCACACUUU | | 2761 | 2783 |
| AD-1949201 | UACACAGGAUCCUAUUCUCAA | | 754 | 774 | UUGAGAAUAGGAUCCUGUGUAUA | | 752 | 774 |
| AD-1949202 | GUGCGGAUUCUUGUGGCUUUA | | 75 | 95 | UAAAGCCACAAGAAUCCGCACAG | | 73 | 95 |
| AD-1949203 | AUCAGGCUGAGGUGUCUUAAA | | 2960 | 2980 | UUUAAGACACCUCAGCCUGAUUA | | 2958 | 2980 |
| AD-1949204 | UUCGAGGUAGAGUUUGAAGGA | | 2666 | 2686 | UCCUUCAAACUCUACCUCGAAAC | | 2664 | 2686 |
| AD-1949205 | UCCUGACCUUAUCCAAGAUGA | | 263 | 283 | UCAUCUUGGAUAAGGUCAGGAUG | | 261 | 283 |
| AD-1949206 | CCUUGAAGGUCACUCUUCCUA | | 564 | 584 | UAGGAAGAGUGACCUUCAAGGCC | | 562 | 584 |
| AD-1949207 | CUGGCUUUCUCCGACUGCUAA | | 1698 | 1718 | UUAGCAGUCGGAGAAAGCCAGAG | | 1696 | 1718 |
| AD-1949209 | UUGCAUUCCCAGUGGUCAAAA | | 2277 | 2297 | UUUUGACCACUGGGAAUGCAAGA | | 2275 | 2297 |
| AD-1949210 | CAGCUGAACAGCCAAAUGCAA | | 2337 | 2357 | UUGCAUUUGGCUGUUCAGCUGCU | | 2335 | 2357 |
| AD-1949211 | AUACCCAGAGCAUUACGUGAA | | 2589 | 2609 | UUCACGUAAUGCUCUGGGUAUCA | | 2587 | 2609 |
| AD-1949212 | CUGGCAGUCUACCAACAGAUA | | 292 | 312 | UAUCUGUUGGUAGACUGCCAGUG | | 290 | 312 |
| AD-1949213 | AGAGCUGCUCUGGAAAAUGUA | | 2916 | 2936 | UACAUUUUCCAGAGCAGCUCUCA | | 2914 | 2936 |
| AD-1949214 | GAGCACCUGCUUCAUGCUCAA | | 2185 | 2205 | UUGAGCAUGAAGCAGGUGCUCAG | | 2183 | 2205 |
| AD-1949215 | GGUGGGAAAUGGUAUGAGCUA | | 2374 | 2394 | UAGCUCAUACCAUUUCCCACCUG | | 2372 | 2394 |
| AD-1949216 | GUGUGGUGGGUUCUUUGGAAA | | 1459 | 1479 | UUUCCAAAGAACCCACCACACAA | | 1457 | 1479 |
| AD-1949217 | GUUCUUGUCUGAUUGGCUCAA | | 1879 | 1899 | UUGAGCCAAUCAGACAAGAACUA | | 1877 | 1899 |
| AD-1949218 | GAUCACAAGGUCACUAGAUGA | | 3145 | 3165 | UCAUCUAGUGACCUUGUGAUCCC | | 3143 | 3165 |
| AD-1949219 | CUCCGGGAUCUUCUUCACGUA | | 367 | 387 | UACGUGAAGAAGAUCCCGGAGGU | | 365 | 387 |
| AD-1949220 | GUCCUGGAAGCUUCAGGCUAA | | 457 | 477 | UUAGCCUGAAGCUUCCAGGACAC | | 455 | 477 |
| AD-1949221 | AUUUCCCUGACUCCUCUAAGA | | 677 | 697 | UCUUAGAGGAGUCAGGGAAAUUG | | 675 | 697 |
| AD-1949222 | UGACUGCGAUCUUCAGAGCAA | | 1760 | 1780 | UUGCUCUGAAGAUCGCAGUCACC | | 1758 | 1780 |
| AD-1949223 | CCAGCUUCUCCAGGCUCUUUA | | 1092 | 1112 | UAAAGAGCCUGGAGAAGCUGGUG | | 1090 | 1112 |
| AD-1949224 | UCUGGCUUCCAGGUAUCUCCA | | 610 | 630 | UGGAGAUACCUGGAAGCCAGAGU | | 608 | 630 |
| AD-1949225 | CCUGGCCAACAUGGUGAAACA | | 3173 | 3193 | UGUUUCACCAUGUUGGCCAGGAU | | 3171 | 3193 |
| AD-1949226 | GUUUUUACUCCAGUGGUGAAA | | 1935 | 1955 | UUUCACCACUGGAGUAAAAACCC | | 1933 | 1955 |
| AD-1949227 | AUGCAUGGUGCAGUUGACAGA | | 2352 | 2372 | UCUGUCAACUGCACCAUGCAUUU | | 2350 | 2372 |
| AD-1949228 | AGAGUACAGUGAGCCAAGAUA | | 3301 | 3321 | UAUCUUGGCUCACUGUACUCUCC | | 3299 | 3321 |
| AD-1949229 | AACACAAAAGUUAGCUGAGCA | | 3208 | 3228 | UGCUCAGCUAACUUUUGUGUUUU | | 3206 | 3228 |
| AD-1949230 | UCCCAGCAACACAAGCUGGAA | | 1019 | 1039 | UUCCAGCUUGUGUUGCUGGGAGU | | 1017 | 1039 |
| AD-1949231 | AGACCUGACAAGCACUGCUAA | | 1981 | 2001 | UUAGCAGUGCUUGUCAGGUCUCA | | 1979 | 2001 |
| AD-1949232 | AUUUCACACACGCAGUCAGUA | | 190 | 210 | UACUGACUGCGUGUGUGAAAUGU | | 188 | 210 |
| AD-1949233 | CAGGUAAUGAGGGACUGGAAA | | 2611 | 2631 | UUUCCAGUCCCUCAUUACCUGGC | | 2609 | 2631 |
| AD-1949234 | UGAGGGAUGUGAAUUGCCUGA | | 2689 | 2709 | UCAGGCAAUUCACAUCCCUCACC | | 2687 | 2709 |
| AD-1949235 | GAUCCUCACCAGUAUGCCUUA | | 309 | 329 | UAAGGCAUACUGGUGAGGAUCUG | | 307 | 329 |
| AD-1949236 | CUGGCCUUCUCUAAGAGCUGA | | 388 | 408 | UCAGCUCUUAGAGAAGGCCAGCA | | 386 | 408 |
| AD-1949237 | GAUUCCCACCAAGGUCUUCAA | | 816 | 836 | UUGAAGACCUUGGUGGGAAUCCC | | 814 | 836 |
| AD-1949238 | AUAUCCAACGACCUGGAGAAA | | 346 | 366 | UUUCUCCAGGUCGUUGGAUAUUU | | 344 | 366 |
| AD-1949239 | CAUGCUGAAGGGACCUUGAAA | | 2821 | 2841 | UUUCAAGGUCCCUUCAGCAUGGC | | 2819 | 2841 |
| AD-1949240 | UGGUGCUAUAGGCUGGAGAAA | | 2213 | 2233 | UUUCUCCAGCCUAUAGCACCAGU | | 2211 | 2233 |
| AD-1949241 | CAUCCACACACGCAGGAACUA | | 1000 | 1020 | UAGUUCCUGCGUGUGUGGAUGUG | | 998 | 1020 |
| AD-1949242 | UUGCCCUGAGUGGAUCUCCAA | | 1315 | 1335 | UUGGAGAUCCACUCAGGGCAACA | | 1313 | 1335 |
| AD-1949243 | CGCACUCACCCAUGUGCCAAA | | 2298 | 2318 | UUUGGCACAUGGGUGAGUGCGGU | | 2296 | 2318 |
| AD-1949244 | GCGGUUGCAAGGCCCAAGAAA | | 19 | 39 | UUUCUUGGGCCUUGCAACCGCUG | | 17 | 39 |
| AD-1949245 | CUGGAGAGAAGUUUCUGGCCA | | 1575 | 1595 | UGGCCAGAAACUUCUCUCCAGGG | | 1573 | 1595 |
| AD-1949246 | GCCUGCAGAGAGAAGCCUGUA | | 2704 | 2724 | UACAGGCUUCUCUCUGCAGGCAA | | 2702 | 2724 |
| AD-1949247 | CCUCUGAAUGGUCCAGGGUUA | | 1797 | 1817 | UAACCCUGGACCAUUCAGAGGGU | | 1795 | 1817 |
| AD-1949248 | CAUUAAGAUUGAGGCCUGCCA | | 2247 | 2267 | UGGCAGGCCUCAAUCUUAAUGUU | | 2245 | 2267 |
| AD-1949249 | GAGACCGAGCGCUUUCUGGAA | | 2636 | 2656 | UUCCAGAAAGCGCUCGGUCUCCC | | 2634 | 2656 |
| AD-1949250 | AGCGAGAGGCAGAGAAAGAAA | | 2782 | 2802 | UUUCUUUCUCUGCCUCUCGCUGU | | 2780 | 2802 |
| AD-1949251 | UGCCACCAUCCUGCUGCUGUA | | 2093 | 2113 | UACAGCAGCAGGAUGGUGGCAGA | | 2091 | 2113 |
| AD-1949252 | CUGCCCUCAGGGAUCUUGCAA | | 2262 | 2282 | UUGCAAGAUCCCUGAGGGCAGGC | | 2260 | 2282 |
| AD-1949253 | CCAAGCUGUGCCCAUCCAAAA | | 114 | 134 | UUUUGGAUGGGCACAGCUUGGAC | | 112 | 134 |
| AD-1949254 | CACAGGGAACCCUGCUUGCAA | | 2417 | 2437 | UUGCAAGCAGGGUUCCCUGUGGG | | 2415 | 2437 |
| AD-1949255 | AUAGCCCAGGUCCUCUGAUAA | | 2572 | 2592 | UUAUCAGAGGACCUGGGCUAUAG | | 2570 | 2592 |
| AD-1949256 | GUCUCUGCAGGACAUGCUGUA | | 513 | 533 | UACAGCAUGUCCUGCAGAGACCC | | 511 | 533 |
| AD-1949257 | ACGGUCCCACACUGGUGACUA | | 1745 | 1765 | UAGUCACCAGUGUGGGACCGUCA | | 1743 | 1765 |
| AD-1949258 | AGUGGGCUGCAUCUGGGAUUA | | 800 | 820 | UAAUCCCAGAUGCAGCCCACUCU | | 798 | 820 |
| AD-1949259 | CCUUCCCACUGGAGGUCACAA | | 2042 | 2062 | UUGUGACCUCCAGUGGGAAGGGC | | 2040 | 2062 |
| AD-1949260 | ACUGCACUCCGGCCUGAUGAA | | 3327 | 3347 | UUCAUCAGGCCGGAGUGCAGUGG | | 3325 | 3347 |
| AD-1949261 | AGUGGCCUGGAGACCUUGGAA | | 424 | 444 | UUCCAAGGUCUCCAGGCCACUGG | | 422 | 444 |
| AD-1949262 | GUCCACCCAGCAAAGAGUGGA | | 785 | 805 | UCCACUCUUUGCUGGGUGGACCC | | 783 | 805 |
| AD-1949263 | GGCUACUCCACAGAGGUGGUA | | 472 | 492 | UACCACCUCUGUGGAGUAGCCUG | | 470 | 492 |
| AD-1949264 | UGGCAGGACCAGGACUAUAGA | | 2556 | 2576 | UCUAUAGUCCUGGUCCUGCCACC | | 2554 | 2576 |
| AD-1949265 | GUUAAGGGAAGGAACUCUGGA | | 595 | 615 | UCCAGAGUUCCUUCCCUUAACGU | | 593 | 615 |
| AD-1949266 | ACAGUGGGUGGUGGAUCUGUA | | 2127 | 2147 | UACAGAUCCACCACCCACUGUGU | | 2125 | 2147 |
| AD-1949267 | GAGCUGGCAAAGGUGGCUCUA | | 1224 | 1244 | UAGAGCCACCUUUGCCAGCUCUU | | 1222 | 1244 |
| AD-1949268 | CCACUCGGGAGGCUGAGACAA | | 3254 | 3274 | UUGUCUCAGCCUCCCGAGUGGCU | | 3252 | 3274 |

TABLE 29
Modified Sense and Antisense Strand Sequences of LEP dsRNA Agents
Comprising an Unsaturated C22 Hydrocarbon Chain Conjugated
to Position 6 on the Sense Strand, Counting from the 5′-end of the Sense Strand

| | | SEQ | | SEQ | | SEQ |
|---|---|---|---|---|---|---|
| Duplex | Sense Strand | ID | | ID | mRNA target | ID |
| Name | Sequence 5′ to 3′ | NO. | Antisense Strand Sequence 5′ to 3′ | NO. | sequence 5′ to 3′ | NO. |
| AD-1949134 | asasugu(Gda)UfuUf | | VPusUfsguuAfuUfCfagaaAfaCfacauuscsu | | AGAAUGUGUUUUCUGAAUAACAU | |
| | UfCfugaauaacsasa | | | | | |
| AD-1949135 | usgsaau(Cda)AfaAf | | VPusAfsaguUfaAfCfugcuUfuGfauucasasg | | CUUGAAUCAAAGCAGUUAACUUU | |
| | GfCfaguuaacususa | | | | | |
| AD-1949136 | gsusgaa(Ada)CfuUf | | VPusUfsuauAfcUfUfaggaAfgUfuucacsasc | | GUGUGAAACUUCCUAAGUAUAAA | |
| | CfCfuaaguauasasa | | | | | |
| AD-1949137 | uscsucg(Uda)AfaCf | | VPusAfsaauGfaAfAfccagUfuAfcgagasgsa | | UCUCUCGUAACUGGUUUCAUUUC | |
| | UfGfguuucauususa | | | | | |
| AD-1949138 | usgscac(Uda)UfuGf | | VPusUfsaaaCfaUfGfuuacAfaAfgugcasasg | | CUUGCACUUUGUAACAUGUUUAC | |
| | UfAfacauguuusasa | | | | | |
| AD-1949139 | csusguc(Cda)AfaGf | | VPusAfsuucAfaGfUfuuccUfuGfgacagsasu | | AUCUGUCCAAGGAAACUUGAAUC | |
| | GfAfaacuugaasusa | | | | | |
| AD-1949140 | usgsaag(Ada)AfaAf | | VPusUfsgcuAfaAfGfgaguUfuUfcuucascsc | | GGUGAAGAAAACUCCUUUAGCAG | |
| | CfUfccuuuagcsasa | | | | | |
| AD-1949141 | gsgsagu(Gda)AfgAf | | VPusUfsaagAfaAfAfugauCfuCfacuccsusu | | AAGGAGUGAGAUCAUUUUCUUAU | |
| | UfCfauuuucuusasa | | | | | |
| AD-1949142 | ususguc(Uda)GfuUf | | VPusUfsaagUfuAfCfaaaaAfcAfgacaascsc | | GGUUGUCUGUUUUUGUAACUUAA | |
| | UfUfuguaacuusasa | | | | | |
| AD-1949143 | gscsaug(Cda)CfaAf | | VPusAfsgaaCfuAfCfaauuUfgGfcaugcsasa | | UUGCAUGCCAAAUUGUAGUUCUU | |
| | AfUfuguaguucsusa | | | | | |
| AD-1949144 | asasggg(Uda)AfaAf | | VPusUfsaucAfaAfCfuucuUfuAfcccuuscsa | | UGAAGGGUAAAGAAGUUUGAUAU | |
| | GfAfaguuugausasa | | | | | |
| AD-1949145 | ususagg(Ada)UfgUf | | VPusUfsuucAfuGfAfaaaaCfaUfccuaasgsc | | GCUUAGGAUGUUUUUCAUGAAAA | |
| | UfUfuucaugaasasa | | | | | |
| AD-1949146 | cscsuca(Uda)CfaAf | | VPusUfsgacAfaUfUfgucuUfgAfugaggsgsu | | ACCCUCAUCAAGACAAUUGUCAC | |
| | GfAfcaauugucsasa | | | | | |
| AD-1949147 | uscsaca(Uda)UfcAf | | VPusUfsuucAfuCfUfuccuGfaAfugugascsc | | GGUCACAUUCAGGAAGAUGAAAG | |
| | GfGfaagaugaasasa | | | | | |
| AD-1949148 | ususugu(Cda)AfaGf | | VPusUfsacaUfaUfGfacacUfuGfacaaasasc | | GUUUUGUCAAGUGUCAUAUGUAG | |
| | UfGfucauaugusasa | | | | | |
| AD-1949149 | cscsuuu(Gda)AfaAf | | VPusCfsaguUfaUfCfuuguUfuCfaaaggsgsa | | UCCCUUUGAAACAAGAUAACUGA | |
| | CfAfagauaacusgsa | | | | | |
| AD-1949150 | csusccu(Uda)UfuGf | | VPusUfsuggUfuUfCfaagcAfaAfaggagsgsc | | GCCUCCUUUUGCUUGAAACCAAA | |
| | CfUfugaaaccasasa | | | | | |
| AD-1949151 | csascca(Gda)GfaUf | | VPusAfsaauGfuCfAfuugaUfcCfuggugsasc | | GUCACCAGGAUCAAUGACAUUUC | |
| | CfAfaugacauususa | | | | | |
| AD-1949152 | csusuca(Ada)AfuCf | | VPusUfsauuCfuGfGfauggAfuUfugaagscsa | | UGCUUCAAAUCCAUCCAGAAUAA | |
| | CfAfuccagaausasa | | | | | |
| AD-1949153 | gsgsgcu(Gda)AfaAf | | VPusCfsaacAfaAfUfggcuUfuCfagcccsusu | | AAGGGCUGAAAGCCAUUUGUUGG | |
| | GfCfcauuuguusgsa | | | | | |
| AD-1949154 | gsasgcu(Gda)AfaCf | | VPusAfscucAfaAfAfugggUfuCfagcucsasg | | CUGAGCUGAACCCAUUUUGAGUG | |
| | CfCfauuuugagsusa | | | | | |
| AD-1949155 | gscsugu(Gda)UfuUf | | VPusUfsgugAfuAfGfcaaaAfaCfacagcsasg | | CUGCUGUGUUUUUGCUAUCACAC | |
| | UfUfgcuaucacsasa | | | | | |
| AD-1949156 | asuscca(Gda)GfaCf | | VPusAfsaauUfgAfCfagagUfcCfuggausasa | | UUAUCCAGGACUCUGUCAAUUUC | |
| | UfCfugucaauususa | | | | | |
| AD-1949157 | ususcca(Gda)AfaAf | | VPusUfsuugGfaUfCfacguUfuCfuggaasgsg | | CCUUCCAGAAACGUGAUCCAAAU | |
| | CfGfugauccaasasa | | | | | |
| AD-1949158 | csasugg(Cda)AfgUf | | VPusCfsaaaUfaGfGfaacaCfuGfccaugsusa | | UACAUGGCAGUGUUCCUAUUUGG | |
| | GfUfuccuauuusgsa | | | | | |
| AD-1949159 | asasgcc(Uda)UfuUf | | VPusGfsuuuUfgUfGfagcaAfaAfggcuusasa | | UUAAGCCUUUUGCUCACAAAACC | |
| | GfCfucacaaaascsa | | | | | |
| AD-1949160 | csusacu(Gda)UfgAf | | VPusUfsguaAfcAfUfcaguCfaCfaguagsasa | | UUCUACUGUGACUGAUGUUACAU | |
| | CfUfgauguuacsasa | | | | | |
| AD-1949161 | usgsgaa(Gda)CfaCf | | VPusUfsaaaUfaAfAfcaugUfgCfuuccasgsc | | GCUGGAAGCACAUGUUUAUUUAU | |
| | AfUfguuuauuusasa | | | | | |
| AD-1949162 | gscsauc(Uda)UfaGf | | VPusUfsauaAfuAfGfaagcUfaAfgaugcscsc | | GGGCAUCUUAGCUUCUAUUAUAG | |
| | CfUfucuauuausasa | | | | | |
| AD-1949163 | ususcag(Cda)CfaUf | | VPusAfsacuCfuUfGfuugaUfgGfcugaasgsa | | UCUUCAGCCAUCAACAAGAGUUG | |
| | CfAfacaagagususa | | | | | |
| AD-1949164 | asgscuc(Ada)CfcCf | | VPusUfsaauGfuUfUfauugGfgUfgagcususc | | GAAGCUCACCCAAUAAACAUUAA | |
| | AfAfuaaacauusasa | | | | | |
| AD-1949165 | usgsgcc(Cda)UfaUf | | VPusAfscauAfgAfAfaagaUfaGfggccasasa | | UUUGGCCCUAUCUUUUCUAUGUC | |
| | CfUfuuucuaugsusa | | | | | |
| AD-1949166 | gsasuca(Cda)AfcUf | | VPusAfsauaAfaCfCfcagaGfuGfugaucsasa | | UUGAUCACACUCUGGGUUUAUUA | |
| | CfUfggguuuaususa | | | | | |
| AD-1949167 | uscsacc(Gda)GfuUf | | VPusAfsaugAfaGfUfccaaAfcCfggugascsu | | AGUCACCGGUUUGGACUUCAUUC | |
| | UfGfgacuucaususa | | | | | |
| AD-1949168 | gsasgcg(Ada)GfaUf | | VPusUfsuuaAfgAfCfggaaUfcUfcgcucsusg | | CAGAGCGAGAUUCCGUCUUAAAA | |
| | UfCfcgucuuaasasa | | | | | |
| AD-1949169 | ususauc(Uda)UfcUf | | VPusUfsaagCfaAfUfugcaGfaAfgauaasgsa | | UCUUAUCUUCUGCAAUUGCUUAG | |
| | GfCfaauugcuusasa | | | | | |
| AD-1949170 | gsasgua(Gda)CfaAf | | VPusUfsucuCfuAfGfaacuUfgCfuacucsusu | | AAGAGUAGCAAGUUCUAGAGAAG | |
| | GfUfucuagagasasa | | | | | |
| AD-1949171 | cscsugu(Uda)UfuGf | | VPusAfsaacCfuUfCfcaacAfaAfacaggscsu | | AGCCUGUUUUGUUGGAAGGUUUG | |
| | UfUfggaagguususa | | | | | |
| AD-1949172 | ascsagg(Ada)GfaAf | | VPusGfsguuUfaAfGfcgauUfcUfccuguscsu | | AGACAGGAGAAUCGCUUAAACCU | |
| | UfCfgcuuaaacscsa | | | | | |
| AD-1949173 | ascsucu(Uda)CfcAf | | VPusUfscuuAfuGfCfcuuuGfgAfagagusgsg | | CCACUCUUCCAAAGGCAUAAGAC | |
| | AfAfggcauaagsasa | | | | | |
| AD-1949174 | gsasugg(Ada)UfuUf | | VPusUfsgcuUfuGfCfuucaAfaUfccaucscsa | | UGGAUGGAUUUGAAGCAAAGCAC | |
| | GfAfagcaaagcsasa | | | | | |
| AD-1949175 | csusccu(Cda)CfaAf | | VPusUfsgacUfuUfCfuguuUfgGfaggagsasc | | GUCUCCUCCAAACAGAAAGUCAC | |
| | AfCfagaaagucsasa | | | | | |
| AD-1949176 | csusgga(Ada)AfaGf | | VPusUfscgaAfaCfUfccucUfuUfuccagsasa | | UUCUGGAAAAGAGGAGUUUCGAG | |
| | AfGfgaguuucgsasa | | | | | |
| AD-1949177 | csasguu(Uda)CfcAf | | VPusAfsucuAfuGfGfgauuGfgAfaacugscsa | | UGCAGUUUCCAAUCCCAUAGAUG | |
| | AfUfcccauagasusa | | | | | |
| AD-1949178 | ascscug(Gda)CfaCf | | VPusAfsauuAfgCfCfauugUfgCfcaggususu | | AAACCUGGCACAAUGGCUAAUUC | |
| | AfAfuggcuaaususa | | | | | |
| AD-1949179 | uscscaa(Gda)GfaCf | | VPusAfsaaaUfaAfCfcuggUfcCfuuggasgsa | | UCUCCAAGGACCAGGUUAUUUUA | |
| | CfAfgguuauuususa | | | | | |
| AD-1949180 | cscsugg(Gda)AfaGf | | VPusAfsaugCfaUfUfuuccUfuCfccaggsasu | | AUCCUGGGAAGGAAAAUGCAUUG | |
| | GfAfaaaugcaususa | | | | | |
| AD-1949181 | asgsucc(Ada)AfgAf | | VPusUfsuuuGfgUfGfucauCfuUfggacususu | | AAAGUCCAAGAUGACACCAAAAC | |
| | UfGfacaccaaasasa | | | | | |
| AD-1949182 | uscscuc(Ada)CfaAf | | VPusUfsgauUfaGfGfugguUfgUfgaggasusc | | GAUCCUCACAACCACCUAAUCAG | |
| | CfCfaccuaaucsasa | | | | | |
| AD-1949183 | asgscaa(Gda)GfcCf | | VPusUfsuggUfaAfUfuuugGfcCfuugcususg | | CAAGCAAGGCCAAAAUUACCAAA | |
| | AfAfaauuaccasasa | | | | | |
| AD-1949184 | csascug(Ada)AfuGf | | VPusUfscacAfuUfGfaggcAfuUfcagugsasg | | CUCACUGAAUGCCUCAAUGUGAC | |
| | CfCfucaaugugsasa | | | | | |
| AD-1949185 | ususaca(Uda)CfaCf | | VPusUfsugcAfaAfCfacugUfgAfuguaascsa | | UGUUACAUCACAGUGUUUGCAAU | |
| | AfGfuguuugcasasa | | | | | |
| AD-1949186 | uscscaa(Gda)GfaGf | | VPusUfscuuCfaUfGfgaacUfcCfuuggasasu | | AUUCCAAGGAGUUCCAUGAAGAC | |
| | UfUfccaugaagsasa | | | | | |
| AD-1949187 | asgsgau(Uda)GfaAf | | VPusAfsugcAfaUfGfcucuUfcAfauccusgsg | | CCAGGAUUGAAGAGCAUUGCAUG | |
| | GfAfgcauugcasusa | | | | | |
| AD-1949188 | asasggg(Ada)GfaAf | | VPusAfsuucUfaGfAfuccuUfcUfcccuuscsu | | AGAAGGGAGAAGGAUCUAGAAUG | |
| | GfGfaucuagaasusa | | | | | |
| AD-1949189 | usasuua(Ada)AfgGf | | VPusUfsacuCfuUfAfacucCfuUfuaauasusc | | GAUAUUAAAGGAGUUAAGAGUAG | |
| | AfGfuuaagagusasa | | | | | |
| AD-1949190 | gsusggu(Cda)UfuUf | | VPusUfsccaUfgAfUfaggaAfaGfaccacsusc | | GAGUGGUCUUUCCUAUCAUGGAG | |
| | CfCfuaucauggsasa | | | | | |
| AD-1949191 | uscsugc(Ada)UfuUf | | VPusCfsaucCfaGfAfauaaAfaUfgcagasasu | | AUUCUGCAUUUUAUUCUGGAUGG | |
| | UfAfuucuggausgsa | | | | | |
| AD-1949192 | uscscug(Cda)AfaGf | | VPusUfsuaaCfgUfAfguccUfuGfcaggasasg | | CUUCCUGCAAGGACUACGUUAAG | |
| | GfAfcuacguuasasa | | | | | |
| AD-1949193 | gsusucc(Cda)UfcUf | | VPusUfsuggAfaUfUfcucaGfaGfggaacscsu | | AGGUUCCCUCUGAGAAUUCCAAG | |
| | GfAfgaauuccasasa | | | | | |
| AD-1949194 | gsasagc(Uda)GfaUf | | VPusUfsgaaGfcAfAfagcaUfcAfgcuucsusc | | GAGAAGCUGAUGCUUUGCUUCAA | |
| | GfCfuuugcuucsasa | | | | | |
| AD-1949195 | asasaua(Cda)AfuAf | | VPusUfsguuAfuGfGfucuuAfuGfuauuususc | | GAAAAUACAUAAGACCAUAACAG | |
| | AfGfaccauaacsasa | | | | | |
| AD-1949196 | cscsuuu(Gda)GfaUf | | VPusUfsuguUfcUfGfgucaUfcCfaaaggscsu | | AGCCUUUGGAUGACCAGAACAAG | |
| | GfAfccagaacasasa | | | | | |
| AD-1949197 | gsgsaga(Uda)GfcAf | | VPusAfscuuUfuAfCfcucuGfcAfucuccsasc | | GUGGAGAUGCAGAGGUAAAAGUG | |
| | GfAfgguaaaagsusa | | | | | |
| AD-1949198 | ususggg(Uda)UfcAf | | VPusUfscuuGfcUfCfagauGfaAfcccaascsc | | GGUUGGGUUCAUCUGAGCAAGAG | |
| | UfCfugagcaagsasa | | | | | |
| AD-1949199 | gscsucu(Cda)CfaGf | | VPusGfsagaGfaAfCfuaacUfgGfagagcscsa | | UGGCUCUCCAGUUAGUUCUCUCG | |
| | UfUfaguucucuscsa | | | | | |
| AD-1949200 | asgsugu(Gda)AfgCf | | VPusUfsguaAfcUfCfacugCfuCfacacususu | | AAAGUGUGAGCAGUGAGUUACAG | |
| | AfGfugaguuacsasa | | | | | |
| AD-1949201 | usascac(Ada)GfgAf | | VPusUfsgagAfaUfAfggauCfcUfguguasusa | | UAUACACAGGAUCCUAUUCUCAC | |
| | UfCfcuauucucsasa | | | | | |
| AD-1949202 | gsusgcg(Gda)AfuUf | | VPusAfsaagCfcAfCfaagaAfuCfcgcacsasg | | CUGUGCGGAUUCUUGUGGCUUUG | |
| | CfUfuguggcuususa | | | | | |
| AD-1949203 | asuscag(Gda)CfuGf | | VPusUfsuaaGfaCfAfccucAfgCfcugaususa | | UAAUCAGGCUGAGGUGUCUUAAG | |
| | AfGfgugucuuasasa | | | | | |
| AD-1949204 | ususcga(Gda)GfuAf | | VPusCfscuuCfaAfAfcucuAfcCfucgaasasc | | GUUUCGAGGUAGAGUUUGAAGGA | |
| | GfAfguuugaagsgsa | | | | | |
| AD-1949205 | uscscug(Ada)CfcUf | | VPusCfsaucUfuGfGfauaaGfgUfcaggasusg | | CAUCCUGACCUUAUCCAAGAUGG | |
| | UfAfuccaagausgsa | | | | | |
| AD-1949206 | cscsuug(Ada)AfgGf | | VPusAfsggaAfgAfGfugacCfuUfcaaggscsc | | GGCCUUGAAGGUCACUCUUCCUG | |
| | UfCfacucuuccsusa | | | | | |
| AD-1949207 | csusggc(Uda)UfuCf | | VPusUfsagcAfgUfCfggagAfaAfgccagsasg | | CUCUGGCUUUCUCCGACUGCUAG | |
| | UfCfcgacugcusasa | | | | | |
| AD-1949209 | ususgca(Uda)UfcCf | | VPusUfsuugAfcCfAfcuggGfaAfugcaasgsa | | UCUUGCAUUCCCAGUGGUCAAAC | |
| | CfAfguggucaasasa | | | | | |
| AD-1949210 | csasgcu(Gda)AfaCf | | VPusUfsgcaUfuUfGfgcugUfuCfagcugscsu | | AGCAGCUGAACAGCCAAAUGCAU | |
| | AfGfccaaaugcsasa | | | | | |
| AD-1949211 | asusacc(Cda)AfgAf | | VPusUfscacGfuAfAfugcuCfuGfgguauscsa | | UGAUACCCAGAGCAUUACGUGAG | |
| | GfCfauuacgugsasa | | | | | |
| AD-1949212 | csusggc(Ada)GfuCf | | VPusAfsucuGfuUfGfguagAfcUfgccagsusg | | CACUGGCAGUCUACCAACAGAUC | |
| | UfAfccaacagasusa | | | | | |
| AD-1949213 | asgsagc(Uda)GfcUf | | VPusAfscauUfuUfCfcagaGfcAfgcucuscsa | | UGAGAGCUGCUCUGGAAAAUGUG | |
| | CfUfggaaaaugsusa | | | | | |
| AD-1949214 | gsasgca(Cda)CfuGf | | VPusUfsgagCfaUfGfaagcAfgGfugcucsasg | | CUGAGCACCUGCUUCAUGCUCAG | |
| | CfUfucaugcucsasa | | | | | |
| AD-1949215 | gsgsugg(Gda)AfaAf | | VPusAfsgcuCfaUfAfccauUfuCfccaccsusg | | CAGGUGGGAAAUGGUAUGAGCUG | |
| | UfGfguaugagcsusa | | | | | |
| AD-1949216 | gsusgug(Gda)UfgGf | | VPusUfsuccAfaAfGfaaccCfaCfcacacsasa | | UUGUGUGGUGGGUUCUUUGGAAG | |
| | GfUfucuuuggasasa | | | | | |
| AD-1949217 | gsusucu(Uda)GfuCf | | VPusUfsgagCfcAfAfucagAfcAfagaacsusa | | UAGUUCUUGUCUGAUUGGCUCAC | |
| | UfGfauuggcucsasa | | | | | |
| AD-1949218 | gsasuca(Cda)AfaGf | | VPusCfsaucUfaGfUfgaccUfuGfugaucscsc | | GGGAUCACAAGGUCACUAGAUGG | |
| | GfUfcacuagausgsa | | | | | |
| AD-1949219 | csusccg(Gda)GfaUf | | VPusAfscguGfaAfGfaagaUfcCfcggagsgsu | | ACCUCCGGGAUCUUCUUCACGUG | |
| | CfUfucuucacgsusa | | | | | |
| AD-1949220 | gsusccu(Gda)GfaAf | | VPusUfsagcCfuGfAfagcuUfcCfaggacsasc | | GUGUCCUGGAAGCUUCAGGCUAC | |
| | GfCfuucaggcusasa | | | | | |
| AD-1949221 | asusuuc(Cda)CfuGf | | VPusCfsuuaGfaGfGfagucAfgGfgaaaususg | | CAAUUUCCCUGACUCCUCUAAGC | |
| | AfCfuccucuaasgsa | | | | | |
| AD-1949222 | usgsacu(Gda)CfgAf | | VPusUfsgcuCfuGfAfagauCfgCfagucascsc | | GGUGACUGCGAUCUUCAGAGCAG | |
| | UfCfuucagagcsasa | | | | | |
| AD-1949223 | cscsagc(Uda)UfcUf | | VPusAfsaagAfgCfCfuggaGfaAfgcuggsusg | | CACCAGCUUCUCCAGGCUCUUUG | |
| | CfCfaggcucuususa | | | | | |
| AD-1949224 | uscsugg(Cda)UfuCf | | VPusGfsgagAfuAfCfcuggAfaGfccagasgsu | | ACUCUGGCUUCCAGGUAUCUCCA | |
| | CfAfgguaucucscsa | | | | | |
| AD-1949225 | cscsugg(Cda)CfaAf | | VPusGfsuuuCfaCfCfauguUfgGfccaggsasu | | AUCCUGGCCAACAUGGUGAAACC | |
| | CfAfuggugaaascsa | | | | | |
| AD-1949226 | gsusuuu(Uda)AfcUf | | VPusUfsucaCfcAfCfuggaGfuAfaaaacscsc | | GGGUUUUUACUCCAGUGGUGAAG | |
| | CfCfaguggugasasa | | | | | |
| AD-1949227 | asusgca(Uda)GfgUf | | VPusCfsuguCfaAfCfugcaCfcAfugcaususu | | AAAUGCAUGGUGCAGUUGACAGC | |
| | GfCfaguugacasgsa | | | | | |
| AD-1949228 | asgsagu(Ada)CfaGf | | VPusAfsucuUfgGfCfucacUfgUfacucuscsc | | GGAGAGUACAGUGAGCCAAGAUC | |
| | UfGfagccaagasusa | | | | | |
| AD-1949229 | asascac(Ada)AfaAf | | VPusGfscucAfgCfUfaacuUfuUfguguususu | | AAAACACAAAAGUUAGCUGAGCG | |
| | GfUfuagcugagscsa | | | | | |
| AD-1949230 | uscscca(Gda)CfaAf | | VPusUfsccaGfcUfUfguguUfgCfugggasgsu | | ACUCCCAGCAACACAAGCUGGAA | |
| | CfAfcaagcuggsasa | | | | | |
| AD-1949231 | asgsacc(Uda)GfaCf | | VPusUfsagcAfgUfGfcuugUfcAfggucuscsa | | UGAGACCUGACAAGCACUGCUAG | |
| | AfAfgcacugcusasa | | | | | |
| AD-1949232 | asusuuc(Ada)CfaCf | | VPusAfscugAfcUfGfcgugUfgUfgaaausgsu | | ACAUUUCACACACGCAGUCAGUC | |
| | AfCfgcagucagsusa | | | | | |
| AD-1949233 | csasggu(Ada)AfuGf | | VPusUfsuccAfgUfCfccucAfuUfaccugsgsc | | GCCAGGUAAUGAGGGACUGGAAC | |
| | AfGfggacuggasasa | | | | | |
| AD-1949234 | usgsagg(Gda)AfuGf | | VPusCfsaggCfaAfUfucacAfuCfccucascsc | | GGUGAGGGAUGUGAAUUGCCUGC | |
| | UfGfaauugccusgsa | | | | | |
| AD-1949235 | gsasucc(Uda)CfaCf | | VPusAfsaggCfaUfAfcuggUfgAfggaucsusg | | CAGAUCCUCACCAGUAUGCCUUC | |
| | CfAfguaugccususa | | | | | |
| AD-1949236 | csusggc(Cda)UfuCf | | VPusCfsagcUfcUfUfagagAfaGfgccagscsa | | UGCUGGCCUUCUCUAAGAGCUGC | |
| | UfCfuaagagcusgsa | | | | | |
| AD-1949237 | gsasuuc(Cda)CfaCf | | VPusUfsgaaGfaCfCfuuggUfgGfgaaucscsc | | GGGAUUCCCACCAAGGUCUUCAG | |
| | CfAfaggucuucsasa | | | | | |
| AD-1949238 | asusauc(Cda)AfaCf | | VPusUfsucuCfcAfGfgucgUfuGfgauaususu | | AAAUAUCCAACGACCUGGAGAAC | |
| | GfAfccuggagasasa | | | | | |
| AD-1949239 | csasugc(Uda)GfaAf | | VPusUfsucaAfgGfUfcccuUfcAfgcaugsgsc | | GCCAUGCUGAAGGGACCUUGAAG | |
| | GfGfgaccuugasasa | | | | | |
| AD-1949240 | usgsgug(Cda)UfaUf | | VPusUfsucuCfcAfGfccuaUfaGfcaccasgsu | | ACUGGUGCUAUAGGCUGGAGAAG | |
| | AfGfgcuggagasasa | | | | | |
| AD-1949241 | csasucc(Ada)CfaCf | | VPusAfsguuCfcUfGfcgugUfgUfggaugsusg | | CACAUCCACACACGCAGGAACUC | |
| | AfCfgcaggaacsusa | | | | | |
| AD-1949242 | ususgcc(Cda)UfgAf | | VPusUfsggaGfaUfCfcacuCfaGfggcaascsa | | UGUUGCCCUGAGUGGAUCUCCAA | |
| | GfUfggaucuccsasa | | | | | |
| AD-1949243 | csgscac(Uda)CfaCf | | VPusUfsuggCfaCfAfugggUfgAfgugcgsgsu | | ACCGCACUCACCCAUGUGCCAAG | |
| | CfCfaugugccasasa | | | | | |
| AD-1949244 | gscsggu(Uda)GfcAf | | VPusUfsucuUfgGfGfccuuGfcAfaccgcsusg | | CAGCGGUUGCAAGGCCCAAGAAG | |
| | AfGfgcccaagasasa | | | | | |
| AD-1949245 | csusgga(Gda)AfgAf | | VPusGfsgccAfgAfAfacuuCfuCfuccagsgsg | | CCCUGGAGAGAAGUUUCUGGCCC | |
| | AfGfuuucuggcscsa | | | | | |
| AD-1949246 | gscscug(Cda)AfgAf | | VPusAfscagGfcUfUfcucuCfuGfcaggcsasa | | UUGCCUGCAGAGAGAAGCCUGUU | |
| | GfAfgaagccugsusa | | | | | |
| AD-1949247 | cscsucu(Gda)AfaUf | | VPusAfsaccCfuGfGfaccaUfuCfagaggsgsu | | ACCCUCUGAAUGGUCCAGGGUUG | |
| | GfGfuccagggususa | | | | | |
| AD-1949248 | csasuua(Ada)GfaUf | | VPusGfsgcaGfgCfCfucaaUfcUfuaaugsusu | | AACAUUAAGAUUGAGGCCUGCCC | |
| | UfGfaggccugcscsa | | | | | |
| AD-1949249 | gsasgac(Cda)GfaGf | | VPusUfsccaGfaAfAfgcgcUfcGfgucucscsc | | GGGAGACCGAGCGCUUUCUGGAA | |
| | CfGfcuuucuggsasa | | | | | |
| AD-1949250 | asgscga(Gda)AfgGf | | VPusUfsucuUfuCfUfcugcCfuCfucgcusgsu | | ACAGCGAGAGGCAGAGAAAGAAG | |
| | CfAfgagaaagasasa | | | | | |
| AD-1949251 | usgscca(Cda)CfaUf | | VPusAfscagCfaGfCfaggaUfgGfuggcasgsa | | UCUGCCACCAUCCUGCUGCUGUG | |
| | CfCfugcugcugsusa | | | | | |
| AD-1949252 | csusgcc(Cda)UfcAf | | VPusUfsgcaAfgAfUfcccuGfaGfggcagsgsc | | GCCUGCCCUCAGGGAUCUUGCAU | |
| | GfGfgaucuugcsasa | | | | | |
| AD-1949253 | cscsaag(Cda)UfgUf | | VPusUfsuugGfaUfGfggcaCfaGfcuuggsasc | | GUCCAAGCUGUGCCCAUCCAAAA | |
| | GfCfccauccaasasa | | | | | |
| AD-1949254 | csascag(Gda)GfaAf | | VPusUfsgcaAfgCfAfggguUfcCfcugugsgsg | | CCCACAGGGAACCCUGCUUGCAC | |
| | CfCfcugcuugcsasa | | | | | |
| AD-1949255 | asusagc(Cda)CfaGf | | VPusUfsaucAfgAfGfgaccUfgGfgcuausasg | | CUAUAGCCCAGGUCCUCUGAUAC | |
| | GfUfccucugausasa | | | | | |
| AD-1949256 | gsuscuc(Uda)GfcAf | | VPusAfscagCfaUfGfuccuGfcAfgagacscsc | | GGGUCUCUGCAGGACAUGCUGUG | |
| | GfGfacaugcugsusa | | | | | |
| AD-1949257 | ascsggu(Cda)CfcAf | | VPusAfsgucAfcCfAfguguGfgGfaccguscsa | | UGACGGUCCCACACUGGUGACUG | |
| | CfAfcuggugacsusa | | | | | |
| AD-1949258 | asgsugg(Gda)CfuGf | | VPusAfsaucCfcAfGfaugcAfgCfccacuscsu | | AGAGUGGGCUGCAUCUGGGAUUC | |
| | CfAfucugggaususa | | | | | |
| AD-1949259 | cscsuuc(Cda)CfaCf | | VPusUfsgugAfcCfUfccagUfgGfgaaggsgsc | | GCCCUUCCCACUGGAGGUCACAU | |
| | UfGfgaggucacsasa | | | | | |
| AD-1949260 | ascsugc(Ada)CfuCf | | VPusUfscauCfaGfGfccggAfgUfgcagusgsg | | CCACUGCACUCCGGCCUGAUGAC | |
| | CfGfgccugaugsasa | | | | | |
| AD-1949261 | asgsugg(Cda)CfuGf | | VPusUfsccaAfgGfUfcuccAfgGfccacusgsg | | CCAGUGGCCUGGAGACCUUGGAC | |
| | GfAfgaccuuggsasa | | | | | |
| AD-1949262 | gsuscca(Cda)CfcAf | | VPusCfscacUfcUfUfugcuGfgGfuggacscsc | | GGGUCCACCCAGCAAAGAGUGGG | |
| | GfCfaaagagugsgsa | | | | | |
| AD-1949263 | gsgscua(Cda)UfcCf | | VPusAfsccaCfcUfCfugugGfaGfuagccsusg | | CAGGCUACUCCACAGAGGUGGUG | |
| | AfCfagagguggsusa | | | | | |
| AD-1949264 | usgsgca(Gda)GfaCf | | VPusCfsuauAfgUfCfcuggUfcCfugccascsc | | GGUGGCAGGACCAGGACUAUAGC | |
| | CfAfggacuauasgsa | | | | | |
| AD-1949265 | gsusuaa(Gda)GfgAf | | VPusCfscagAfgUfUfccuuCfcCfuuaacsgsu | | ACGUUAAGGGAAGGAACUCUGGC | |
| | AfGfgaacucugsgsa | | | | | |
| AD-1949266 | ascsagu(Gda)GfgUf | | VPusAfscagAfuCfCfaccaCfcCfacugusgsu | | ACACAGUGGGUGGUGGAUCUGUC | |
| | GfGfuggaucugsusa | | | | | |
| AD-1949267 | gsasgcu(Gda)GfcAf | | VPusAfsgagCfcAfCfcuuuGfcCfagcucsusu | | AAGAGCUGGCAAAGGUGGCUCUC | |
| | AfAfgguggcucsusa | | | | | |
| AD-1949268 | cscsacu(Cda)GfgGf | | VPusUfsgucUfcAfGfccucCfcGfaguggscsu | | AGCCACUCGGGAGGCUGAGACAG | |
| | AfGfgcugagacsasa | | | | | |

TABLE 30
Unmodified Sense and Antisense Strand Sequences of LEP dsRNA Agents

| | | SEQ | NM_000230.3 | | | SEQ | NM_000230.3 | |
|---|---|---|---|---|---|---|---|---|
| Duplex | Sense Strand | ID | Start | End | Antisense Strand | ID | Start | End |
| Name | Sequence 5′ to 3′ | NO: | Position | Position | Sequence 5′ to 3′ | NO: | Position | Position |
| AD-1945272 | GCGGUUGCAAGGCCCAAGAAA | | 19 | 39 | UUUCUUGGGCCUUGCAACCGCUG | | 17 | 39 |
| AD-1945298 | CCUGGGAAGGAAAAUGCAUUA | | 45 | 65 | UAAUGCAUUUUCCUUCCCAGGAU | | 43 | 65 |
| AD-1945308 | GUGCGGAUUCUUGUGGCUUUA | | 75 | 95 | UAAAGCCACAAGAAUCCGCACAG | | 73 | 95 |
| AD-1945327 | UGGCCCUAUCUUUUCUAUGUA | | 94 | 114 | UACAUAGAAAAGAUAGGGCCAAA | | 92 | 114 |
| AD-1945347 | CCAAGCUGUGCCCAUCCAAAA | | 114 | 134 | UUUUGGAUGGGCACAGCUUGGAC | | 112 | 134 |
| AD-1945372 | AGUCCAAGAUGACACCAAAAA | | 135 | 155 | UUUUUGGUGUCAUCUUGGACUUU | | 133 | 155 |
| AD-1945386 | CCUCAUCAAGACAAUUGUCAA | | 156 | 176 | UUGACAAUUGUCUUGAUGAGGGU | | 154 | 176 |
| AD-1945401 | AUUUCACACACGCAGUCAGUA | | 190 | 210 | UACUGACUGCGUGUGUGAAAUGU | | 188 | 210 |
| AD-1945422 | CUCCUCCAAACAGAAAGUCAA | | 210 | 230 | UUGACUUUCUGUUUGGAGGAGAC | | 208 | 230 |
| AD-1945439 | UCACCGGUUUGGACUUCAUUA | | 227 | 247 | UAAUGAAGUCCAAACCGGUGACU | | 225 | 247 |
| AD-1945454 | UCCUGACCUUAUCCAAGAUGA | | 263 | 283 | UCAUCUUGGAUAAGGUCAGGAUG | | 261 | 283 |
| AD-1945483 | CUGGCAGUCUACCAACAGAUA | | 292 | 312 | UAUCUGUUGGUAGACUGCCAGUG | | 290 | 312 |
| AD-1945500 | GAUCCUCACCAGUAUGCCUUA | | 309 | 329 | UAAGGCAUACUGGUGAGGAUCUG | | 307 | 329 |
| AD-1945518 | UUCCAGAAACGUGAUCCAAAA | | 327 | 347 | UUUUGGAUCACGUUUCUGGAAGG | | 325 | 347 |
| AD-1945537 | AUAUCCAACGACCUGGAGAAA | | 346 | 366 | UUUCUCCAGGUCGUUGGAUAUUU | | 344 | 366 |
| AD-1945558 | CUCCGGGAUCUUCUUCACGUA | | 367 | 387 | UACGUGAAGAAGAUCCCGGAGGU | | 365 | 387 |
| AD-1945579 | CUGGCCUUCUCUAAGAGCUGA | | 388 | 408 | UCAGCUCUUAGAGAAGGCCAGCA | | 386 | 408 |
| AD-1945611 | AGUGGCCUGGAGACCUUGGAA | | 424 | 444 | UUCCAAGGUCUCCAGGCCACUGG | | 422 | 444 |
| AD-1945624 | GUCCUGGAAGCUUCAGGCUAA | | 457 | 477 | UUAGCCUGAAGCUUCCAGGACAC | | 455 | 477 |
| AD-1945639 | GGCUACUCCACAGAGGUGGUA | | 472 | 492 | UACCACCUCUGUGGAGUAGCCUG | | 470 | 492 |
| AD-1945658 | GUCUCUGCAGGACAUGCUGUA | | 513 | 533 | UACAGCAUGUCCUGCAGAGACCC | | 511 | 533 |
| AD-1945708 | CCUUGAAGGUCACUCUUCCUA | | 564 | 584 | UAGGAAGAGUGACCUUCAAGGCC | | 562 | 584 |
| AD-1945724 | UCCUGCAAGGACUACGUUAAA | | 580 | 600 | UUUAACGUAGUCCUUGCAGGAAG | | 578 | 600 |
| AD-1945739 | GUUAAGGGAAGGAACUCUGGA | | 595 | 615 | UCCAGAGUUCCUUCCCUUAACGU | | 593 | 615 |
| AD-1945754 | UCUGGCUUCCAGGUAUCUCCA | | 610 | 630 | UGGAGAUACCUGGAAGCCAGAGU | | 608 | 630 |
| AD-1945774 | AGGAUUGAAGAGCAUUGCAUA | | 630 | 650 | UAUGCAAUGCUCUUCAAUCCUGG | | 628 | 650 |
| AD-1945785 | AUCCAGGACUCUGUCAAUUUA | | 661 | 681 | UAAAUUGACAGAGUCCUGGAUAA | | 659 | 681 |
| AD-1945801 | AUUUCCCUGACUCCUCUAAGA | | 677 | 697 | UCUUAGAGGAGUCAGGGAAAUUG | | 675 | 697 |
| AD-1945823 | ACUCUUCCAAAGGCAUAAGAA | | 699 | 719 | UUCUUAUGCCUUUGGAAGAGUGG | | 697 | 719 |
| AD-1945851 | CUCCUUUUGCUUGAAACCAAA | | 727 | 747 | UUUGGUUUCAAGCAAAAGGAGGC | | 725 | 747 |
| AD-1945878 | UACACAGGAUCCUAUUCUCAA | | 754 | 774 | UUGAGAAUAGGAUCCUGUGUAUA | | 752 | 774 |
| AD-1945888 | GUCCACCCAGCAAAGAGUGGA | | 785 | 805 | UCCACUCUUUGCUGGGUGGACCC | | 783 | 805 |
| AD-1945903 | AGUGGGCUGCAUCUGGGAUUA | | 800 | 820 | UAAUCCCAGAUGCAGCCCACUCU | | 798 | 820 |
| AD-1945919 | GAUUCCCACCAAGGUCUUCAA | | 816 | 836 | UUGAAGACCUUGGUGGGAAUCCC | | 814 | 836 |
| AD-1945935 | UUCAGCCAUCAACAAGAGUUA | | 832 | 852 | UAACUCUUGUUGAUGGCUGAAGA | | 830 | 852 |
| AD-1945947 | CACUGAAUGCCUCAAUGUGAA | | 882 | 902 | UUCACAUUGAGGCAUUCAGUGAG | | 880 | 902 |
| AD-1945966 | CCUUUGGAUGACCAGAACAAA | | 941 | 961 | UUUGUUCUGGUCAUCCAAAGGCU | | 939 | 961 |
| AD-1945987 | GUUCCCUCUGAGAAUUCCAAA | | 962 | 982 | UUUGGAAUUCUCAGAGGGAACCU | | 960 | 982 |
| AD-1946002 | UCCAAGGAGUUCCAUGAAGAA | | 977 | 997 | UUCUUCAUGGAACUCCUUGGAAU | | 975 | 997 |
| AD-1946025 | CAUCCACACACGCAGGAACUA | | 1000 | 1020 | UAGUUCCUGCGUGUGUGGAUGUG | | 998 | 1020 |
| AD-1946044 | UCCCAGCAACACAAGCUGGAA | | 1019 | 1039 | UUCCAGCUUGUGUUGCUGGGAGU | | 1017 | 1039 |
| AD-1946060 | UGGAAGCACAUGUUUAUUUAA | | 1035 | 1055 | UUAAAUAAACAUGUGCUUCCAGC | | 1033 | 1055 |
| AD-1946076 | UCUGCAUUUUAUUCUGGAUGA | | 1056 | 1076 | UCAUCCAGAAUAAAAUGCAGAAU | | 1054 | 1076 |
| AD-1946092 | GAUGGAUUUGAAGCAAAGCAA | | 1072 | 1092 | UUGCUUUGCUUCAAAUCCAUCCA | | 1070 | 1092 |
| AD-1946112 | CCAGCUUCUCCAGGCUCUUUA | | 1092 | 1112 | UAAAGAGCCUGGAGAAGCUGGUG | | 1090 | 1112 |
| AD-1946129 | CAGUUUCCAAUCCCAUAGAUA | | 1146 | 1166 | UAUCUAUGGGAUUGGAAACUGCA | | 1144 | 1166 |
| AD-1946159 | GAGCUGAACCCAUUUUGAGUA | | 1176 | 1196 | UACUCAAAAUGGGUUCAGCUCAG | | 1174 | 1196 |
| AD-1946189 | UUGGGUUCAUCUGAGCAAGAA | | 1206 | 1226 | UUCUUGCUCAGAUGAACCCAACC | | 1204 | 1226 |
| AD-1946207 | GAGCUGGCAAAGGUGGCUCUA | | 1224 | 1244 | UAGAGCCACCUUUGCCAGCUCUU | | 1222 | 1244 |
| AD-1946222 | GCUCUCCAGUUAGUUCUCUCA | | 1239 | 1259 | UGAGAGAACUAACUGGAGAGCCA | | 1237 | 1259 |
| AD-1946238 | UCUCGUAACUGGUUUCAUUUA | | 1255 | 1275 | UAAAUGAAACCAGUUACGAGAGA | | 1253 | 1275 |
| AD-1946258 | CUACUGUGACUGAUGUUACAA | | 1275 | 1295 | UUGUAACAUCAGUCACAGUAGAA | | 1273 | 1295 |
| AD-1946273 | UUACAUCACAGUGUUUGCAAA | | 1290 | 1310 | UUUGCAAACACUGUGAUGUAACA | | 1288 | 1310 |
| AD-1946298 | UUGCCCUGAGUGGAUCUCCAA | | 1315 | 1335 | UUGGAGAUCCACUCAGGGCAACA | | 1313 | 1335 |
| AD-1946314 | UCCAAGGACCAGGUUAUUUUA | | 1331 | 1351 | UAAAAUAACCUGGUCCUUGGAGA | | 1329 | 1351 |
| AD-1946336 | UUUGUCAAGUGUCAUAUGUAA | | 1363 | 1383 | UUACAUAUGACACUUGACAAAAC | | 1361 | 1383 |
| AD-1946366 | AAGGGAGAAGGAUCUAGAAUA | | 1419 | 1439 | UAUUCUAGAUCCUUCUCCCUUCU | | 1417 | 1439 |
| AD-1946383 | AAUGUGUUUUCUGAAUAACAA | | 1436 | 1456 | UUGUUAUUCAGAAAACACAUUCU | | 1434 | 1456 |
| AD-1946402 | GUGUGGUGGGUUCUUUGGAAA | | 1459 | 1479 | UUUCCAAAGAACCCACCACACAA | | 1457 | 1479 |
| AD-1946422 | GGAGUGAGAUCAUUUUCUUAA | | 1479 | 1499 | UUAAGAAAAUGAUCUCACUCCUU | | 1477 | 1499 |
| AD-1946438 | UUAUCUUCUGCAAUUGCUUAA | | 1496 | 1516 | UUAAGCAAUUGCAGAAGAUAAGA | | 1494 | 1516 |
| AD-1946455 | UUAGGAUGUUUUUCAUGAAAA | | 1513 | 1533 | UUUUCAUGAAAAACAUCCUAAGC | | 1511 | 1533 |
| AD-1946472 | CUGGAGAGAAGUUUCUGGCCA | | 1575 | 1595 | UGGCCAGAAACUUCUCUCCAGGG | | 1573 | 1595 |
| AD-1946496 | GAAGCUGAUGCUUUGCUUCAA | | 1619 | 1639 | UUGAAGCAAAGCAUCAGCUUCUC | | 1617 | 1639 |
| AD-1946511 | CUUCAAAUCCAUCCAGAAUAA | | 1634 | 1654 | UUAUUCUGGAUGGAUUUGAAGCA | | 1632 | 1654 |
| AD-1946540 | GGGCUGAAAGCCAUUUGUUGA | | 1663 | 1683 | UCAACAAAUGGCUUUCAGCCCUU | | 1661 | 1683 |
| AD-1946555 | CUGGCUUUCUCCGACUGCUAA | | 1698 | 1718 | UUAGCAGUCGGAGAAAGCCAGAG | | 1696 | 1718 |
| AD-1946579 | GUGGUCUUUCCUAUCAUGGAA | | 1722 | 1742 | UUCCAUGAUAGGAAAGACCACUC | | 1720 | 1742 |
| AD-1946602 | ACGGUCCCACACUGGUGACUA | | 1745 | 1765 | UAGUCACCAGUGUGGGACCGUCA | | 1743 | 1765 |
| AD-1946617 | UGACUGCGAUCUUCAGAGCAA | | 1760 | 1780 | UUGCUCUGAAGAUCGCAGUCACC | | 1758 | 1780 |
| AD-1946634 | CCUCUGAAUGGUCCAGGGUUA | | 1797 | 1817 | UAACCCUGGACCAUUCAGAGGGU | | 1795 | 1817 |
| AD-1946654 | GAUCACACUCUGGGUUUAUUA | | 1817 | 1837 | UAAUAAACCCAGAGUGUGAUCAA | | 1815 | 1837 |
| AD-1946675 | CAUGGCAGUGUUCCUAUUUGA | | 1838 | 1858 | UCAAAUAGGAACACUGCCAUGUA | | 1836 | 1858 |
| AD-1946681 | GCAUGCCAAAUUGUAGUUCUA | | 1864 | 1884 | UAGAACUACAAUUUGGCAUGCAA | | 1862 | 1884 |
| AD-1946696 | GUUCUUGUCUGAUUGGCUCAA | | 1879 | 1899 | UUGAGCCAAUCAGACAAGAACUA | | 1877 | 1899 |
| AD-1946720 | AGCAAGGCCAAAAUUACCAAA | | 1903 | 1923 | UUUGGUAAUUUUGGCCUUGCUUG | | 1901 | 1923 |
| AD-1946729 | GUUUUUACUCCAGUGGUGAAA | | 1935 | 1955 | UUUCACCACUGGAGUAAAAACCC | | 1933 | 1955 |
| AD-1946745 | UGAAGAAAACUCCUUUAGCAA | | 1951 | 1971 | UUGCUAAAGGAGUUUUCUUCACC | | 1949 | 1971 |
| AD-1946775 | AGACCUGACAAGCACUGCUAA | | 1981 | 2001 | UUAGCAGUGCUUGUCAGGUCUCA | | 1979 | 2001 |
| AD-1946816 | CCUUCCCACUGGAGGUCACAA | | 2042 | 2062 | UUGUGACCUCCAGUGGGAAGGGC | | 2040 | 2062 |
| AD-1946831 | UCACAUUCAGGAAGAUGAAAA | | 2057 | 2077 | UUUUCAUCUUCCUGAAUGUGACC | | 2055 | 2077 |
| AD-1946845 | UGCCACCAUCCUGCUGCUGUA | | 2093 | 2113 | UACAGCAGCAGGAUGGUGGCAGA | | 2091 | 2113 |
| AD-1946860 | GCUGUGUUUUUGCUAUCACAA | | 2108 | 2128 | UUGUGAUAGCAAAAACACAGCAG | | 2106 | 2128 |
| AD-1946879 | ACAGUGGGUGGUGGAUCUGUA | | 2127 | 2147 | UACAGAUCCACCACCCACUGUGU | | 2125 | 2147 |
| AD-1946895 | CUGUCCAAGGAAACUUGAAUA | | 2143 | 2163 | UAUUCAAGUUUCCUUGGACAGAU | | 2141 | 2163 |
| AD-1946910 | UGAAUCAAAGCAGUUAACUUA | | 2158 | 2178 | UAAGUUAACUGCUUUGAUUCAAG | | 2156 | 2178 |
| AD-1946937 | GAGCACCUGCUUCAUGCUCAA | | 2185 | 2205 | UUGAGCAUGAAGCAGGUGCUCAG | | 2183 | 2205 |
| AD-1946965 | UGGUGCUAUAGGCUGGAGAAA | | 2213 | 2233 | UUUCUCCAGCCUAUAGCACCAGU | | 2211 | 2233 |
| AD-1946984 | AGCUCACCCAAUAAACAUUAA | | 2232 | 2252 | UUAAUGUUUAUUGGGUGAGCUUC | | 2230 | 2252 |
| AD-1946998 | CAUUAAGAUUGAGGCCUGCCA | | 2247 | 2267 | UGGCAGGCCUCAAUCUUAAUGUU | | 2245 | 2267 |
| AD-1947013 | CUGCCCUCAGGGAUCUUGCAA | | 2262 | 2282 | UUGCAAGAUCCCUGAGGGCAGGC | | 2260 | 2282 |
| AD-1947028 | UUGCAUUCCCAGUGGUCAAAA | | 2277 | 2297 | UUUUGACCACUGGGAAUGCAAGA | | 2275 | 2297 |
| AD-1947049 | CGCACUCACCCAUGUGCCAAA | | 2298 | 2318 | UUUGGCACAUGGGUGAGUGCGGU | | 2296 | 2318 |
| AD-1947068 | CAGCUGAACAGCCAAAUGCAA | | 2337 | 2357 | UUGCAUUUGGCUGUUCAGCUGCU | | 2335 | 2357 |
| AD-1947083 | AUGCAUGGUGCAGUUGACAGA | | 2352 | 2372 | UCUGUCAACUGCACCAUGCAUUU | | 2350 | 2372 |
| AD-1947105 | GGUGGGAAAUGGUAUGAGCUA | | 2374 | 2394 | UAGCUCAUACCAUUUCCCACCUG | | 2372 | 2394 |
| AD-1947113 | CACAGGGAACCCUGCUUGCAA | | 2417 | 2437 | UUGCAAGCAGGGUUCCCUGUGGG | | 2415 | 2437 |
| AD-1947129 | UGCACUUUGUAACAUGUUUAA | | 2433 | 2453 | UUAAACAUGUUACAAAGUGCAAG | | 2431 | 2453 |
| AD-1947158 | GCAUCUUAGCUUCUAUUAUAA | | 2462 | 2482 | UUAUAAUAGAAGCUAAGAUGCCC | | 2460 | 2482 |
| AD-1947186 | CCUUUGAAACAAGAUAACUGA | | 2490 | 2510 | UCAGUUAUCUUGUUUCAAAGGGA | | 2488 | 2510 |
| AD-1947204 | AAAUACAUAAGACCAUAACAA | | 2527 | 2547 | UUGUUAUGGUCUUAUGUAUUUUC | | 2525 | 2547 |
| AD-1947233 | UGGCAGGACCAGGACUAUAGA | | 2556 | 2576 | UCUAUAGUCCUGGUCCUGCCACC | | 2554 | 2576 |
| AD-1947249 | AUAGCCCAGGUCCUCUGAUAA | | 2572 | 2592 | UUAUCAGAGGACCUGGGCUAUAG | | 2570 | 2592 |
| AD-1947266 | AUACCCAGAGCAUUACGUGAA | | 2589 | 2609 | UUCACGUAAUGCUCUGGGUAUCA | | 2587 | 2609 |
| AD-1947288 | CAGGUAAUGAGGGACUGGAAA | | 2611 | 2631 | UUUCCAGUCCCUCAUUACCUGGC | | 2609 | 2631 |
| AD-1947313 | GAGACCGAGCGCUUUCUGGAA | | 2636 | 2656 | UUCCAGAAAGCGCUCGGUCUCCC | | 2634 | 2656 |
| AD-1947328 | CUGGAAAAGAGGAGUUUCGAA | | 2651 | 2671 | UUCGAAACUCCUCUUUUCCAGAA | | 2649 | 2671 |
| AD-1947343 | UUCGAGGUAGAGUUUGAAGGA | | 2666 | 2686 | UCCUUCAAACUCUACCUCGAAAC | | 2664 | 2686 |
| AD-1947366 | UGAGGGAUGUGAAUUGCCUGA | | 2689 | 2709 | UCAGGCAAUUCACAUCCCUCACC | | 2687 | 2709 |
| AD-1947381 | GCCUGCAGAGAGAAGCCUGUA | | 2704 | 2724 | UACAGGCUUCUCUCUGCAGGCAA | | 2702 | 2724 |
| AD-1947396 | CCUGUUUUGUUGGAAGGUUUA | | 2719 | 2739 | UAAACCUUCCAACAAAACAGGCU | | 2717 | 2739 |
| AD-1947423 | GGAGAUGCAGAGGUAAAAGUA | | 2746 | 2766 | UACUUUUACCUCUGCAUCUCCAC | | 2744 | 2766 |
| AD-1947440 | AGUGUGAGCAGUGAGUUACAA | | 2763 | 2783 | UUGUAACUCACUGCUCACACUUU | | 2761 | 2783 |
| AD-1947459 | AGCGAGAGGCAGAGAAAGAAA | | 2782 | 2802 | UUUCUUUCUCUGCCUCUCGCUGU | | 2780 | 2802 |
| AD-1947489 | CAUGCUGAAGGGACCUUGAAA | | 2821 | 2841 | UUUCAAGGUCCCUUCAGCAUGGC | | 2819 | 2841 |
| AD-1947507 | AAGGGUAAAGAAGUUUGAUAA | | 2839 | 2859 | UUAUCAAACUUCUUUACCCUUCA | | 2837 | 2859 |
| AD-1947522 | UAUUAAAGGAGUUAAGAGUAA | | 2857 | 2877 | UUACUCUUAACUCCUUUAAUAUC | | 2855 | 2877 |
| AD-1947537 | GAGUAGCAAGUUCUAGAGAAA | | 2872 | 2892 | UUUCUCUAGAACUUGCUACUCUU | | 2870 | 2892 |
| AD-1947581 | AGAGCUGCUCUGGAAAAUGUA | | 2916 | 2936 | UACAUUUUCCAGAGCAGCUCUCA | | 2914 | 2936 |
| AD-1947609 | UCCUCACAACCACCUAAUCAA | | 2944 | 2964 | UUGAUUAGGUGGUUGUGAGGAUC | | 2942 | 2964 |
| AD-1947625 | AUCAGGCUGAGGUGUCUUAAA | | 2960 | 2980 | UUUAAGACACCUCAGCCUGAUUA | | 2958 | 2980 |
| AD-1947643 | AAGCCUUUUGCUCACAAAACA | | 2978 | 2998 | UGUUUUGUGAGCAAAAGGCUUAA | | 2976 | 2998 |
| AD-1947661 | ACCUGGCACAAUGGCUAAUUA | | 2996 | 3016 | UAAUUAGCCAUUGUGCCAGGUUU | | 2994 | 3016 |
| AD-1947689 | GUGAAACUUCCUAAGUAUAAA | | 3024 | 3044 | UUUAUACUUAGGAAGUUUCACAC | | 3022 | 3044 |
| AD-1947713 | UUGUCUGUUUUUGUAACUUAA | | 3048 | 3068 | UUAAGUUACAAAAACAGACAACC | | 3046 | 3068 |
| AD-1947724 | CCUGGCCAACAUGGUGAAACA | | 15292 | 15312 | UGUUUCACCAUGUUGGCCAGGAU | | 15290 | 15312 |
| AD-1947747 | GAUCACAAGGUCACUAGAUGA | | 3145 | 3165 | UCAUCUAGUGACCUUGUGAUCCC | | 3143 | 3165 |
| AD-1947787 | AACACAAAAGUUAGCUGAGCA | | 3208 | 3228 | UGCUCAGCUAACUUUUGUGUUUU | | 3206 | 3228 |
| AD-1947821 | CCACUCGGGAGGCUGAGACAA | | 3254 | 3274 | UUGUCUCAGCCUCCCGAGUGGCU | | 3252 | 3274 |
| AD-1947838 | ACAGGAGAAUCGCUUAAACCA | | 3271 | 3291 | UGGUUUAAGCGAUUCUCCUGUCU | | 3269 | 3291 |
| AD-1947868 | AGAGUACAGUGAGCCAAGAUA | | 3301 | 3321 | UAUCUUGGCUCACUGUACUCUCC | | 3299 | 3321 |
| AD-1947891 | ACUGCACUCCGGCCUGAUGAA | | 3327 | 3347 | UUCAUCAGGCCGGAGUGCAGUGG | | 3325 | 3347 |
| AD-1947913 | GAGCGAGAUUCCGUCUUAAAA | | 3349 | 3369 | UUUUAAGACGGAAUCUCGCUCUG | | 3347 | 3369 |

TABLE 31
Modified Sense and Antisense Strand Sequences of LEP
dsRNA Agents Comprising a GalNAc Conjugate Targeting Ligand

| | | SEQ | | SEQ | | SEQ |
|---|---|---|---|---|---|---|
| Duplex | Sense Strand | ID | Antisense Strand | ID | mRNA target | ID |
| Name | Sequence 5′ to 3′ | NO. | Sequence 5′ to 3′ | NO. | sequence 5′ to 3′ | NO. |
| AD-1945272 | gscsgguuGfcAfAfGf | | VPusUfsucuUfgGfGfccuuGfcAfaccgcsusg | | CAGCGGUUGCAAGGCCCAAGAAG | |
| | gcccaagaaaL96 | | | | | |
| AD-1945298 | cscsugggAfaGfGfAf | | VPusAfsaugCfaUfUfuuccUfuCfccaggsasu | | AUCCUGGGAAGGAAAAUGCAUUG | |
| | aaaugcauuaL96 | | | | | |
| AD-1945308 | gsusgcggAfuUfCfUf | | VPusAfsaagCfcAfCfaagaAfuCfcgcacsasg | | CUGUGCGGAUUCUUGUGGCUUUG | |
| | uguggcuuuaL96 | | | | | |
| AD-1945327 | usgsgcccUfaUfCfUf | | VPusAfscauAfgAfAfaagaUfaGfggccasasa | | UUUGGCCCUAUCUUUUCUAUGUC | |
| | uuucuauguaL96 | | | | | |
| AD-1945347 | cscsaagcUfgUfGfCf | | VPusUfsuugGfaUfGfggcaCfaGfcuuggsasc | | GUCCAAGCUGUGCCCAUCCAAAA | |
| | ccauccaaaaL96 | | | | | |
| AD-1945372 | asgsuccaAfgAfUfGf | | VPusUfsuuuGfgUfGfucauCfuUfggacususu | | AAAGUCCAAGAUGACACCAAAAC | |
| | acaccaaaaaL96 | | | | | |
| AD-1945386 | cscsucauCfaAfGfAf | | VPusUfsgacAfaUfUfgucuUfgAfugaggsgsu | | ACCCUCAUCAAGACAAUUGUCAC | |
| | caauugucaaL96 | | | | | |
| AD-1945401 | asusuucaCfaCfAfCf | | VPusAfscugAfcUfGfcgugUfgUfgaaausgsu | | ACAUUUCACACACGCAGUCAGUC | |
| | gcagucaguaL96 | | | | | |
| AD-1945422 | csusccucCfaAfAfCf | | VPusUfsgacUfuUfCfuguuUfgGfaggagsasc | | GUCUCCUCCAAACAGAAAGUCAC | |
| | agaaagucaaL96 | | | | | |
| AD-1945439 | uscsaccgGfuUfUfGf | | VPusAfsaugAfaGfUfccaaAfcCfggugascsu | | AGUCACCGGUUUGGACUUCAUUC | |
| | gacuucauuaL96 | | | | | |
| AD-1945454 | uscscugaCfcUfUfAf | | VPusCfsaucUfuGfGfauaaGfgUfcaggasusg | | CAUCCUGACCUUAUCCAAGAUGG | |
| | uccaagaugaL96 | | | | | |
| AD-1945483 | csusggcaGfuCfUfAf | | VPusAfsucuGfuUfGfguagAfcUfgccagsusg | | CACUGGCAGUCUACCAACAGAUC | |
| | ccaacagauaL96 | | | | | |
| AD-1945500 | gsasuccuCfaCfCfAf | | VPusAfsaggCfaUfAfcuggUfgAfggaucsusg | | CAGAUCCUCACCAGUAUGCCUUC | |
| | guaugccuuaL96 | | | | | |
| AD-1945518 | ususccagAfaAfCfGf | | VPusUfsuugGfaUfCfacguUfuCfuggaasgsg | | CCUUCCAGAAACGUGAUCCAAAU | |
| | ugauccaaaaL96 | | | | | |
| AD-1945537 | asusauccAfaCfGfAf | | VPusUfsucuCfcAfGfgucgUfuGfgauaususu | | AAAUAUCCAACGACCUGGAGAAC | |
| | ccuggagaaaL96 | | | | | |
| AD-1945558 | csusccggGfaUfCfUf | | VPusAfscguGfaAfGfaagaUfcCfcggagsgsu | | ACCUCCGGGAUCUUCUUCACGUG | |
| | ucuucacguaL96 | | | | | |
| AD-1945579 | csusggccUfuCfUfCf | | VPusCfsagcUfcUfUfagagAfaGfgccagscsa | | UGCUGGCCUUCUCUAAGAGCUGC | |
| | uaagagcugaL96 | | | | | |
| AD-1945611 | asgsuggcCfuGfGfAf | | VPusUfsccaAfgGfUfcuccAfgGfccacusgsg | | CCAGUGGCCUGGAGACCUUGGAC | |
| | gaccuuggaaL96 | | | | | |
| AD-1945624 | gsusccugGfaAfGfCf | | VPusUfsagcCfuGfAfagcuUfcCfaggacsasc | | GUGUCCUGGAAGCUUCAGGCUAC | |
| | uucaggcuaaL96 | | | | | |
| AD-1945639 | gsgscuacUfcCfAfCf | | VPusAfsccaCfcUfCfugugGfaGfuagccsusg | | CAGGCUACUCCACAGAGGUGGUG | |
| | agaggugguaL96 | | | | | |
| AD-1945658 | gsuscucuGfcAfGfGf | | VPusAfscagCfaUfGfuccuGfcAfgagacscsc | | GGGUCUCUGCAGGACAUGCUGUG | |
| | acaugcuguaL96 | | | | | |
| AD-1945708 | cscsuugaAfgGfUfCf | | VPusAfsggaAfgAfGfugacCfuUfcaaggscsc | | GGCCUUGAAGGUCACUCUUCCUG | |
| | acucuuccuaL96 | | | | | |
| AD-1945724 | uscscugcAfaGfGfAf | | VPusUfsuaaCfgUfAfguccUfuGfcaggasasg | | CUUCCUGCAAGGACUACGUUAAG | |
| | cuacguuaaaL96 | | | | | |
| AD-1945739 | gsusuaagGfgAfAfGf | | VPusCfscagAfgUfUfccuuCfcCfuuaacsgsu | | ACGUUAAGGGAAGGAACUCUGGC | |
| | gaacucuggaL96 | | | | | |
| AD-1945754 | uscsuggcUfuCfCfAf | | VPusGfsgagAfuAfCfcuggAfaGfccagasgsu | | ACUCUGGCUUCCAGGUAUCUCCA | |
| | gguaucuccaL96 | | | | | |
| AD-1945774 | asgsgauuGfaAfGfAf | | VPusAfsugcAfaUfGfcucuUfcAfauccusgsg | | CCAGGAUUGAAGAGCAUUGCAUG | |
| | gcauugcauaL96 | | | | | |
| AD-1945785 | asusccagGfaCfUfCf | | VPusAfsaauUfgAfCfagagUfcCfuggausasa | | UUAUCCAGGACUCUGUCAAUUUC | |
| | ugucaauuuaL96 | | | | | |
| AD-1945801 | asusuuccCfuGfAfCf | | VPusCfsuuaGfaGfGfagucAfgGfgaaaususg | | CAAUUUCCCUGACUCCUCUAAGC | |
| | uccucuaagaL96 | | | | | |
| AD-1945823 | ascsucuuCfcAfAfAf | | VPusUfscuuAfuGfCfcuuuGfgAfagagusgsg | | CCACUCUUCCAAAGGCAUAAGAC | |
| | ggcauaagaaL96 | | | | | |
| AD-1945851 | csusccuuUfuGfCfUf | | VPusUfsuggUfuUfCfaagcAfaAfaggagsgsc | | GCCUCCUUUUGCUUGAAACCAAA | |
| | ugaaaccaaaL96 | | | | | |
| AD-1945878 | usascacaGfgAfUfCf | | VPusUfsgagAfaUfAfggauCfcUfguguasusa | | UAUACACAGGAUCCUAUUCUCAC | |
| | cuauucucaaL96 | | | | | |
| AD-1945888 | gsusccacCfcAfGfCf | | VPusCfscacUfcUfUfugcuGfgGfuggacscsc | | GGGUCCACCCAGCAAAGAGUGGG | |
| | aaagaguggaL96 | | | | | |
| AD-1945903 | asgsugggCfuGfCfAf | | VPusAfsaucCfcAfGfaugcAfgCfccacuscsu | | AGAGUGGGCUGCAUCUGGGAUUC | |
| | ucugggauuaL96 | | | | | |
| AD-1945919 | gsasuuccCfaCfCfAf | | VPusUfsgaaGfaCfCfuuggUfgGfgaaucscsc | | GGGAUUCCCACCAAGGUCUUCAG | |
| | aggucuucaaL96 | | | | | |
| AD-1945935 | ususcagcCfaUfCfAf | | VPusAfsacuCfuUfGfuugaUfgGfcugaasgsa | | UCUUCAGCCAUCAACAAGAGUUG | |
| | acaagaguuaL96 | | | | | |
| AD-1945947 | csascugaAfuGfCfCf | | VPusUfscacAfuUfGfaggcAfuUfcagugsasg | | CUCACUGAAUGCCUCAAUGUGAC | |
| | ucaaugugaaL96 | | | | | |
| AD-1945966 | cscsuuugGfaUfGfAf | | VPusUfsuguUfcUfGfgucaUfcCfaaaggscsu | | AGCCUUUGGAUGACCAGAACAAG | |
| | ccagaacaaaL96 | | | | | |
| AD-1945987 | gsusucccUfcUfGfAf | | VPusUfsuggAfaUfUfcucaGfaGfggaacscsu | | AGGUUCCCUCUGAGAAUUCCAAG | |
| | gaauuccaaaL96 | | | | | |
| AD-1946002 | uscscaagGfaGfUfUf | | VPusUfscuuCfaUfGfgaacUfcCfuuggasasu | | AUUCCAAGGAGUUCCAUGAAGAC | |
| | ccaugaagaaL96 | | | | | |
| AD-1946025 | csasuccaCfaCfAfCf | | VPusAfsguuCfcUfGfcgugUfgUfggaugsusg | | CACAUCCACACACGCAGGAACUC | |
| | gcaggaacuaL96 | | | | | |
| AD-1946044 | uscsccagCfaAfCfAf | | VPusUfsccaGfcUfUfguguUfgCfugggasgsu | | ACUCCCAGCAACACAAGCUGGAA | |
| | caagcuggaaL96 | | | | | |
| AD-1946060 | usgsgaagCfaCfAfUf | | VPusUfsaaaUfaAfAfcaugUfgCfuuccasgsc | | GCUGGAAGCACAUGUUUAUUUAU | |
| | guuuauuuaaL96 | | | | | |
| AD-1946076 | uscsugcaUfuUfUfAf | | VPusCfsaucCfaGfAfauaaAfaUfgcagasasu | | AUUCUGCAUUUUAUUCUGGAUGG | |
| | uucuggaugaL96 | | | | | |
| AD-1946092 | gsasuggaUfuUfGfAf | | VPusUfsgcuUfuGfCfuucaAfaUfccaucscsa | | UGGAUGGAUUUGAAGCAAAGCAC | |
| | agcaaagcaaL96 | | | | | |
| AD-1946112 | cscsagcuUfcUfCfCf | | VPusAfsaagAfgCfCfuggaGfaAfgcuggsusg | | CACCAGCUUCUCCAGGCUCUUUG | |
| | aggcucuuuaL96 | | | | | |
| AD-1946129 | csasguuuCfcAfAfUf | | VPusAfsucuAfuGfGfgauuGfgAfaacugscsa | | UGCAGUUUCCAAUCCCAUAGAUG | |
| | cccauagauaL96 | | | | | |
| AD-1946159 | gsasgcugAfaCfCfCf | | VPusAfscucAfaAfAfugggUfuCfagcucsasg | | CUGAGCUGAACCCAUUUUGAGUG | |
| | auuuugaguaL96 | | | | | |
| AD-1946189 | ususggguUfcAfUfCf | | VPusUfscuuGfcUfCfagauGfaAfcccaascsc | | GGUUGGGUUCAUCUGAGCAAGAG | |
| | ugagcaagaaL96 | | | | | |
| AD-1946207 | gsasgcugGfcAfAfAf | | VPusAfsgagCfcAfCfcuuuGfcCfagcucsusu | | AAGAGCUGGCAAAGGUGGCUCUC | |
| | gguggcucuaL96 | | | | | |
| AD-1946222 | gscsucucCfaGfUfUf | | VPusGfsagaGfaAfCfuaacUfgGfagagcscsa | | UGGCUCUCCAGUUAGUUCUCUCG | |
| | aguucucucaL96 | | | | | |
| AD-1946238 | uscsucguAfaCfUfGf | | VPusAfsaauGfaAfAfccagUfuAfcgagasgsa | | UCUCUCGUAACUGGUUUCAUUUC | |
| | guuucauuuaL96 | | | | | |
| AD-1946258 | csusacugUfgAfCfUf | | VPusUfsguaAfcAfUfcaguCfaCfaguagsasa | | UUCUACUGUGACUGAUGUUACAU | |
| | gauguuacaaL96 | | | | | |
| AD-1946273 | ususacauCfaCfAfGf | | VPusUfsugcAfaAfCfacugUfgAfuguaascsa | | UGUUACAUCACAGUGUUUGCAAU | |
| | uguuugcaaaL96 | | | | | |
| AD-1946298 | ususgcccUfgAfGfUf | | VPusUfsggaGfaUfCfcacuCfaGfggcaascsa | | UGUUGCCCUGAGUGGAUCUCCAA | |
| | ggaucuccaaL96 | | | | | |
| AD-1946314 | uscscaagGfaCfCfAf | | VPusAfsaaaUfaAfCfcuggUfcCfuuggasgsa | | UCUCCAAGGACCAGGUUAUUUUA | |
| | gguuauuuuaL96 | | | | | |
| AD-1946336 | ususugucAfaGfUfGf | | VPusUfsacaUfaUfGfacacUfuGfacaaasasc | | GUUUUGUCAAGUGUCAUAUGUAG | |
| | ucauauguaaL96 | | | | | |
| AD-1946366 | asasgggaGfaAfGfGf | | VPusAfsuucUfaGfAfuccuUfcUfcccuuscsu | | AGAAGGGAGAAGGAUCUAGAAUG | |
| | aucuagaauaL96 | | | | | |
| AD-1946383 | asasugugUfuUfUfCf | | VPusUfsguuAfuUfCfagaaAfaCfacauuscsu | | AGAAUGUGUUUUCUGAAUAACAU | |
| | ugaauaacaaL96 | | | | | |
| AD-1946402 | gsusguggUfgGfGfUf | | VPusUfsuccAfaAfGfaaccCfaCfcacacsasa | | UUGUGUGGUGGGUUCUUUGGAAG | |
| | ucuuuggaaaL96 | | | | | |
| AD-1946422 | gsgsagugAfgAfUfCf | | VPusUfsaagAfaAfAfugauCfuCfacuccsusu | | AAGGAGUGAGAUCAUUUUCUUAU | |
| | auuuucuuaaL96 | | | | | |
| AD-1946438 | ususaucuUfcUfGfCf | | VPusUfsaagCfaAfUfugcaGfaAfgauaasgsa | | UCUUAUCUUCUGCAAUUGCUUAG | |
| | aauugcuuaaL96 | | | | | |
| AD-1946455 | ususaggaUfgUfUfUf | | VPusUfsuucAfuGfAfaaaaCfaUfccuaasgsc | | GCUUAGGAUGUUUUUCAUGAAAA | |
| | uucaugaaaaL96 | | | | | |
| AD-1946472 | csusggagAfgAfAfGf | | VPusGfsgccAfgAfAfacuuCfuCfuccagsgsg | | CCCUGGAGAGAAGUUUCUGGCCC | |
| | uuucuggccaL96 | | | | | |
| AD-1946496 | gsasagcuGfaUfGfCf | | VPusUfsgaaGfcAfAfagcaUfcAfgcuucsusc | | GAGAAGCUGAUGCUUUGCUUCAA | |
| | uuugcuucaaL96 | | | | | |
| AD-1946511 | csusucaaAfuCfCfAf | | VPusUfsauuCfuGfGfauggAfuUfugaagscsa | | UGCUUCAAAUCCAUCCAGAAUAA | |
| | uccagaauaaL96 | | | | | |
| AD-1946540 | gsgsgcugAfaAfGfCf | | VPusCfsaacAfaAfUfggcuUfuCfagcccsusu | | AAGGGCUGAAAGCCAUUUGUUGG | |
| | cauuuguugaL96 | | | | | |
| AD-1946555 | csusggcuUfuCfUfCf | | VPusUfsagcAfgUfCfggagAfaAfgccagsasg | | CUCUGGCUUUCUCCGACUGCUAG | |
| | cgacugcuaaL96 | | | | | |
| AD-1946579 | gsusggucUfuUfCfCf | | VPusUfsccaUfgAfUfaggaAfaGfaccacsusc | | GAGUGGUCUUUCCUAUCAUGGAG | |
| | uaucauggaaL96 | | | | | |
| AD-1946602 | ascsggucCfcAfCfAf | | VPusAfsgucAfcCfAfguguGfgGfaccguscsa | | UGACGGUCCCACACUGGUGACUG | |
| | cuggugacuaL96 | | | | | |
| AD-1946617 | usgsacugCfgAfUfCf | | VPusUfsgcuCfuGfAfagauCfgCfagucascsc | | GGUGACUGCGAUCUUCAGAGCAG | |
| | uucagagcaaL96 | | | | | |
| AD-1946634 | cscsucugAfaUfGfGf | | VPusAfsaccCfuGfGfaccaUfuCfagaggsgsu | | ACCCUCUGAAUGGUCCAGGGUUG | |
| | uccaggguuaL96 | | | | | |
| AD-1946654 | gsasucacAfcUfCfUf | | VPusAfsauaAfaCfCfcagaGfuGfugaucsasa | | UUGAUCACACUCUGGGUUUAUUA | |
| | ggguuuauuaL96 | | | | | |
| AD-1946675 | csasuggcAfgUfGfUf | | VPusCfsaaaUfaGfGfaacaCfuGfccaugsusa | | UACAUGGCAGUGUUCCUAUUUGG | |
| | uccuauuugaL96 | | | | | |
| AD-1946681 | gscsaugcCfaAfAfUf | | VPusAfsgaaCfuAfCfaauuUfgGfcaugcsasa | | UUGCAUGCCAAAUUGUAGUUCUU | |
| | uguaguucuaL96 | | | | | |
| AD-1946696 | gsusucuuGfuCfUfGf | | VPusUfsgagCfcAfAfucagAfcAfagaacsusa | | UAGUUCUUGUCUGAUUGGCUCAC | |
| | auuggcucaaL96 | | | | | |
| AD-1946720 | asgscaagGfcCfAfAf | | VPusUfsuggUfaAfUfuuugGfcCfuugcususg | | CAAGCAAGGCCAAAAUUACCAAA | |
| | aauuaccaaaL96 | | | | | |
| AD-1946729 | gsusuuuuAfcUfCfCf | | VPusUfsucaCfcAfCfuggaGfuAfaaaacscsc | | GGGUUUUUACUCCAGUGGUGAAG | |
| | aguggugaaaL96 | | | | | |
| AD-1946745 | usgsaagaAfaAfCfUf | | VPusUfsgcuAfaAfGfgaguUfuUfcuucascsc | | GGUGAAGAAAACUCCUUUAGCAG | |
| | ccuuuagcaaL96 | | | | | |
| AD-1946775 | asgsaccuGfaCfAfAf | | VPusUfsagcAfgUfGfcuugUfcAfggucuscsa | | UGAGACCUGACAAGCACUGCUAG | |
| | gcacugcuaaL96 | | | | | |
| AD-1946816 | cscsuuccCfaCfUfGf | | VPusUfsgugAfcCfUfccagUfgGfgaaggsgsc | | GCCCUUCCCACUGGAGGUCACAU | |
| | gaggucacaaL96 | | | | | |
| AD-1946831 | uscsacauUfcAfGfGf | | VPusUfsuucAfuCfUfuccuGfaAfugugascsc | | GGUCACAUUCAGGAAGAUGAAAG | |
| | aagaugaaaaL96 | | | | | |
| AD-1946845 | usgsccacCfaUfCfCf | | VPusAfscagCfaGfCfaggaUfgGfuggcasgsa | | UCUGCCACCAUCCUGCUGCUGUG | |
| | ugcugcuguaL96 | | | | | |
| AD-1946860 | gscsugugUfuUfUfUf | | VPusUfsgugAfuAfGfcaaaAfaCfacagcsasg | | CUGCUGUGUUUUUGCUAUCACAC | |
| | gcuaucacaaL96 | | | | | |
| AD-1946879 | ascsagugGfgUfGfGf | | VPusAfscagAfuCfCfaccaCfcCfacugusgsu | | ACACAGUGGGUGGUGGAUCUGUC | |
| | uggaucuguaL96 | | | | | |
| AD-1946895 | csusguccAfaGfGfAf | | VPusAfsuucAfaGfUfuuccUfuGfgacagsasu | | AUCUGUCCAAGGAAACUUGAAUC | |
| | aacuugaauaL96 | | | | | |
| AD-1946910 | usgsaaucAfaAfGfCf | | VPusAfsaguUfaAfCfugcuUfuGfauucasasg | | CUUGAAUCAAAGCAGUUAACUUU | |
| | aguuaacuuaL96 | | | | | |
| AD-1946937 | gsasgcacCfuGfCfUf | | VPusUfsgagCfaUfGfaagcAfgGfugcucsasg | | CUGAGCACCUGCUUCAUGCUCAG | |
| | ucaugcucaaL96 | | | | | |
| AD-1946965 | usgsgugcUfaUfAfGf | | VPusUfsucuCfcAfGfccuaUfaGfcaccasgsu | | ACUGGUGCUAUAGGCUGGAGAAG | |
| | gcuggagaaaL96 | | | | | |
| AD-1946984 | asgscucaCfcCfAfAf | | VPusUfsaauGfuUfUfauugGfgUfgagcususc | | GAAGCUCACCCAAUAAACAUUAA | |
| | uaaacauuaaL96 | | | | | |
| AD-1946998 | csasuuaaGfaUfUfGf | | VPusGfsgcaGfgCfCfucaaUfcUfuaaugsusu | | AACAUUAAGAUUGAGGCCUGCCC | |
| | aggccugccaL96 | | | | | |
| AD-1947013 | csusgcccUfcAfGfGf | | VPusUfsgcaAfgAfUfcccuGfaGfggcagsgsc | | GCCUGCCCUCAGGGAUCUUGCAU | |
| | gaucuugcaaL96 | | | | | |
| AD-1947028 | ususgcauUfcCfCfAf | | VPusUfsuugAfcCfAfcuggGfaAfugcaasgsa | | UCUUGCAUUCCCAGUGGUCAAAC | |
| | guggucaaaaL96 | | | | | |
| AD-1947049 | csgscacuCfaCfCfCf | | VPusUfsuggCfaCfAfugggUfgAfgugcgsgsu | | ACCGCACUCACCCAUGUGCCAAG | |
| | augugccaaaL96 | | | | | |
| AD-1947068 | csasgcugAfaCfAfGf | | VPusUfsgcaUfuUfGfgcugUfuCfagcugscsu | | AGCAGCUGAACAGCCAAAUGCAU | |
| | ccaaaugcaaL96 | | | | | |
| AD-1947083 | asusgcauGfgUfGfCf | | VPusCfsuguCfaAfCfugcaCfcAfugcaususu | | AAAUGCAUGGUGCAGUUGACAGC | |
| | aguugacagaL96 | | | | | |
| AD-1947105 | gsgsugggAfaAfUfGf | | VPusAfsgcuCfaUfAfccauUfuCfccaccsusg | | CAGGUGGGAAAUGGUAUGAGCUG | |
| | guaugagcuaL96 | | | | | |
| AD-1947113 | csascaggGfaAfCfCf | | VPusUfsgcaAfgCfAfggguUfcCfcugugsgsg | | CCCACAGGGAACCCUGCUUGCAC | |
| | cugcuugcaaL96 | | | | | |
| AD-1947129 | usgscacuUfuGfUfAf | | VPusUfsaaaCfaUfGfuuacAfaAfgugcasasg | | CUUGCACUUUGUAACAUGUUUAC | |
| | acauguuuaaL96 | | | | | |
| AD-1947158 | gscsaucuUfaGfCfUf | | VPusUfsauaAfuAfGfaagcUfaAfgaugescsc | | GGGCAUCUUAGCUUCUAUUAUAG | |
| | ucuauuauaaL96 | | | | | |
| AD-1947186 | cscsuuugAfaAfCfAf | | VPusCfsaguUfaUfCfuuguUfuCfaaaggsgsa | | UCCCUUUGAAACAAGAUAACUGA | |
| | agauaacugaL96 | | | | | |
| AD-1947204 | asasauacAfuAfAfGf | | VPusUfsguuAfuGfGfucuuAfuGfuauuususc | | GAAAAUACAUAAGACCAUAACAG | |
| | accauaacaaL96 | | | | | |
| AD-1947233 | usgsgcagGfaCfCfAf | | VPusCfsuauAfgUfCfcuggUfcCfugccascsc | | GGUGGCAGGACCAGGACUAUAGC | |
| | ggacuauagaL96 | | | | | |
| AD-1947249 | asusagccCfaGfGfUf | | VPusUfsaucAfgAfGfgaccUfgGfgcuausasg | | CUAUAGCCCAGGUCCUCUGAUAC | |
| | ccucugauaaL96 | | | | | |
| AD-1947266 | asusacccAfgAfGfCf | | VPusUfscacGfuAfAfugcuCfuGfgguauscsa | | UGAUACCCAGAGCAUUACGUGAG | |
| | auuacgugaaL96 | | | | | |
| AD-1947288 | csasgguaAfuGfAfGf | | VPusUfsuccAfgUfCfccucAfuUfaccugsgsc | | GCCAGGUAAUGAGGGACUGGAAC | |
| | ggacuggaaaL96 | | | | | |
| AD-1947313 | gsasgaccGfaGfCfGf | | VPusUfsccaGfaAfAfgcgcUfcGfgucucscsc | | GGGAGACCGAGCGCUUUCUGGAA | |
| | cuuucuggaaL96 | | | | | |
| AD-1947328 | csusggaaAfaGfAfGf | | VPusUfscgaAfaCfUfccucUfuUfuccagsasa | | UUCUGGAAAAGAGGAGUUUCGAG | |
| | gaguuucgaaL96 | | | | | |
| AD-1947343 | ususcgagGfuAfGfAf | | VPusCfscuuCfaAfAfcucuAfcCfucgaasasc | | GUUUCGAGGUAGAGUUUGAAGGA | |
| | guuugaaggaL96 | | | | | |
| AD-1947366 | usgsagggAfuGfUfGf | | VPusCfsaggCfaAfUfucacAfuCfccucascsc | | GGUGAGGGAUGUGAAUUGCCUGC | |
| | aauugccugaL96 | | | | | |
| AD-1947381 | gscscugcAfgAfGfAf | | VPusAfscagGfcUfUfcucuCfuGfcaggcsasa | | UUGCCUGCAGAGAGAAGCCUGUU | |
| | gaagccuguaL96 | | | | | |
| AD-1947396 | cscsuguuUfuGfUfUf | | VPusAfsaacCfuUfCfcaacAfaAfacaggscsu | | AGCCUGUUUUGUUGGAAGGUUUG | |
| | ggaagguuuaL96 | | | | | |
| AD-1947423 | gsgsagauGfcAfGfAf | | VPusAfscuuUfuAfCfcucuGfcAfucuccsasc | | GUGGAGAUGCAGAGGUAAAAGUG | |
| | gguaaaaguaL96 | | | | | |
| AD-1947440 | asgsugugAfgCfAfGf | | VPusUfsguaAfcUfCfacugCfuCfacacususu | | AAAGUGUGAGCAGUGAGUUACAG | |
| | ugaguuacaaL96 | | | | | |
| AD-1947459 | asgscgagAfgGfCfAf | | VPusUfsucuUfuCfUfcugcCfuCfucgcusgsu | | ACAGCGAGAGGCAGAGAAAGAAG | |
| | gagaaagaaaL96 | | | | | |
| AD-1947489 | csasugcuGfaAfGfGf | | VPusUfsucaAfgGfUfcccuUfcAfgcaugsgsc | | GCCAUGCUGAAGGGACCUUGAAG | |
| | gaccuugaaaL96 | | | | | |
| AD-1947507 | asasggguAfaAfGfAf | | VPusUfsaucAfaAfCfuucuUfuAfcccuuscsa | | UGAAGGGUAAAGAAGUUUGAUAU | |
| | aguuugauaaL96 | | | | | |
| AD-1947522 | usasuuaaAfgGfAfGf | | VPusUfsacuCfuUfAfacucCfuUfuaauasusc | | GAUAUUAAAGGAGUUAAGAGUAG | |
| | uuaagaguaaL96 | | | | | |
| AD-1947537 | gsasguagCfaAfGfUf | | VPusUfsucuCfuAfGfaacuUfgCfuacucsusu | | AAGAGUAGCAAGUUCUAGAGAAG | |
| | ucuagagaaaL96 | | | | | |
| AD-1947581 | asgsagcuGfcUfCfUf | | VPusAfscauUfuUfCfcagaGfcAfgcucuscsa | | UGAGAGCUGCUCUGGAAAAUGUG | |
| | ggaaaauguaL96 | | | | | |
| AD-1947609 | uscscucaCfaAfCfCf | | VPusUfsgauUfaGfGfugguUfgUfgaggasusc | | GAUCCUCACAACCACCUAAUCAG | |
| | accuaaucaaL96 | | | | | |
| AD-1947625 | asuscaggCfuGfAfGf | | VPusUfsuaaGfaCfAfccucAfgCfcugaususa | | UAAUCAGGCUGAGGUGUCUUAAG | |
| | gugucuuaaaL96 | | | | | |
| AD-1947643 | asasgccuUfuUfGfCf | | VPusGfsuuuUfgUfGfagcaAfaAfggcuusasa | | UUAAGCCUUUUGCUCACAAAACC | |
| | ucacaaaacaL96 | | | | | |
| AD-1947661 | ascscuggCfaCfAfAf | | VPusAfsauuAfgCfCfauugUfgCfcaggususu | | AAACCUGGCACAAUGGCUAAUUC | |
| | uggcuaauuaL96 | | | | | |
| AD-1947689 | gsusgaaaCfuUfCfCf | | VPusUfsuauAfcUfUfaggaAfgUfuucacsasc | | GUGUGAAACUUCCUAAGUAUAAA | |
| | uaaguauaaaL96 | | | | | |
| AD-1947713 | ususgucuGfuUfUfUf | | VPusUfsaagUfuAfCfaaaaAfcAfgacaascsc | | GGUUGUCUGUUUUUGUAACUUAA | |
| | uguaacuuaaL96 | | | | | |
| AD-1947724 | cscsuggcCfaAfCfAf | | VPusGfsuuuCfaCfCfauguUfgGfccaggsasu | | AGCCUGGCCAACAUGGUGAAACC | |
| | uggugaaacaL96 | | | | | |
| AD-1947747 | gsasucacAfaGfGfUf | | VPusCfsaucUfaGfUfgaccUfuGfugaucscsc | | GGGAUCACAAGGUCACUAGAUGG | |
| | cacuagaugaL96 | | | | | |
| AD-1947787 | asascacaAfaAfGfUf | | VPusGfscucAfgCfUfaacuUfuUfguguususu | | AAAACACAAAAGUUAGCUGAGCG | |
| | uagcugagcaL96 | | | | | |
| AD-1947821 | cscsacucGfgGfAfGf | | VPusUfsgucUfcAfGfccucCfcGfaguggscsu | | AGCCACUCGGGAGGCUGAGACAG | |
| | gcugagacaaL96 | | | | | |
| AD-1947838 | ascsaggaGfaAfUfCf | | VPusGfsguuUfaAfGfcgauUfcUfccuguscsu | | AGACAGGAGAAUCGCUUAAACCU | |
| | gcuuaaaccaL96 | | | | | |
| AD-1947868 | asgsaguaCfaGfUfGf | | VPusAfsucuUfgGfCfucacUfgUfacucuscsc | | GGAGAGUACAGUGAGCCAAGAUC | |
| | agccaagauaL96 | | | | | |
| AD-1947891 | ascsugcaCfuCfCfGf | | VPusUfscauCfaGfGfccggAfgUfgcagusgsg | | CCACUGCACUCCGGCCUGAUGAC | |
| | gccugaugaaL96 | | | | | |
| AD-1947913 | gsasgcgaGfaUfUfCf | | VPusUfsuuaAfgAfCfggaaUfcUfcgcucsusg | | CAGAGCGAGAUUCCGUCUUAAAA | |
| | cgucuuaaaaL96 | | | | | |

TABLE 32
Unmodified Sense and Antisense Strand Sequences of LEP dsRNA Agents Comprising
a GalNAc Conjugate Targeting Ligand NM_008493.3

| | | SEQ | NM_008493.3 | | | SEQ | NM_008493.3 | |
|---|---|---|---|---|---|---|---|---|
| | Sense Strand | ID | Start | End | Antisense Strand | ID | Start | End |
| Duplex Name | Sequence 5′ to 3′ | NO: | Position | Position | Sequence 5′ to 3′ | NO: | Position | Position |
| AD-1646889 | CACCAGGAUCAAUGACAUUUA | | 176 | 196 | UAAAUGUCAUUGAUCCUGGUGAC | | 174 | 196 |

TABLE 33
Modified Sense and Antisense Strand Sequences of LEP dsRNA Agents
Comprising a GalNAc Conjugate Targeting Ligand

| | | SEQ | | SEQ | | SEQ |
|---|---|---|---|---|---|---|
| Duplex | Sense Strand | ID | Antisense Strand | ID | mRNA target | ID |
| Name | Sequence 5′ to 3′ | NO. | Sequence 5′ to 3′ | NO. | sequence 5′ to 3′ | NO. |
| AD-1646889 | csasccagGfaUfCfAf | | VPusAfsaauGfuCfAfu | | GUCACCAGGAUCAAUGACAUUUC | |
| | augacauuuaL96 | | ugaUfcCfuggugsasc | | | |

TABLE 34
Unmodified Sense and Antisense Strand Sequences of LEP dsRNA Agents
Comprising an Unsaturated C22 Hydrocarbon Chain Conjugated to
Position 6 on the Sense Strand, Counting from the 5′-end of the Sense Strand

| | | SEQ | NM_08493.3 | | | SEQ | NM_008493.3 | |
|---|---|---|---|---|---|---|---|---|
| Duplex | Sense Strand | ID | Start | End | Antisense Strand | ID | Start | End |
| Name | Sequence 5′ to 3′ | NO: | Position | Position | Sequence 5′ to 3′ | NO: | Position | Position |
| AD-1888031 | UUGAAGUGUAGUUUUAUACAA | | | | UUGUAUAAAACUACACUUCAAGC | | | |
| AD-1888032 | GUGACUGGUUUUGUUUCUAUA | | | | UAUAGAAACAAAACCAGUCACCA | | | |

TABLE 35
Modified Sense and Antisense Strand Sequences of LEP dsRNA Agents
Comprising an Unsaturated C22 Hydrocarbon Chain Conjugated
to Position 6 on the Sense Strand, Counting from the 5′-end of the Sense Strand

| | | SEQ | | SEQ |
|---|---|---|---|---|
| | | ID | | ID |
| Duplex Name | Sense Strand Sequence 5′ to 3′ | NO. | Antisense Strand Sequence 5′ to 3′ | NO. |
| AD-1888031 | ususgaa(Gda)UfgUfAfGfuuuuauacsasa | | VPusUfsguaUfaAfAfacuaCfaCfuucaasgsc | |
| AD-1888032 | gsusgac(Uda)GfgUfUfUfuguuucuasusa | | VPusAfuagAfaAfCfaaaaCfcAfgucacscsa | |

### Example 15: In Vitro Screening of dsRNA Agents Targeting Leptin

### In Vitro Dual-Luciferase and Endogenous Screening Assays

Hepa1-6 cells cells were transfected by adding 50 μL of siRNA duplexes and 75 ng of a plasmid, comprising partial sequences of human LEP (NM_000230.3), per well along with 100 μL of Opti-MEM plus 0.5 μL of Lipofectamine 2000 per well (Invitrogen, Carlsbad CA. cat #13778-150) and then incubated at room temperature for 15 minutes. The mixture was then added to the cells which are re-suspended in 35 μL of fresh complete media. The transfected cells were incubated at 37° C. in an atmosphere of 5% CO2. Single-dose experiments were performed at 10 nM.

Twenty-four hours after the siRNAs and pV205 plasmid are transfected; Firefly (transfection control) and Renilla (fused to LEP target sequence) luciferase were measured. First, media was removed from cells. Then Firefly luciferase activity was measured by adding 75 μL of Dual-Glo® Luciferase Reagent equal to the culture medium volume to each well and mix. The mixture was incubated at room temperature for 30 minutes before luminescense (500 nm) was measured on a Spectramax (Molecular Devices) to detect the Firefly luciferase signal. Renilla luciferase activity was measured by adding 75 μL of room temperature of Dual-Glo® Stop & Glo® Reagent to each well and the plates were incubated for 10-15 minutes before luminescence was again measured to determine the Renilla luciferase signal. The Dual-Glo® Stop & Glo® Reagent quenches the firefly luciferase signal and sustained luminescence for the Renilla luciferase reaction. siRNA activity was determined by normalizing the Renilla (LEP) signal to the Firefly (control) signal within each well. The magnitude of siRNA activity was then assessed relative to cells that were transfected with the same vector but were not treated with siRNA or were treated with a non-targeting siRNA. All transfections were done with n=4.

Total RNA Isolation Using DYNABEADS mRNA Isolation Kit (Invitrogen™, Part #: 610-12)

Cells were lysed in 75 μl of Lysis/Binding Buffer containing 3 μL of beads per well and mixed for 10 minutes on an electrostatic shaker. The washing steps were automated on a Biotek EL406, using a magnetic plate support. Beads were washed (in 90 L) once in Buffer A, once in Buffer B, and twice in Buffer E, with aspiration steps in between. Following a final aspiration, complete 10 μL RT mixture was added to each well, as described below.

cDNA Synthesis Using ABI High Capacity cDNA Reverse Transcription Kit (Applied Biosystems, Foster City, CA, Cat #4368813)

A master mix of 1 μl 10× Buffer, 0.4 μl 25× dNTPs, 1 μl Random primers, 0.5 μl Reverse Transcriptase, 0.5 μl RNase inhibitor and 6.6 μl of H₂O per reaction were added per well. Plates were sealed, agitated for 10 minutes on an electrostatic shaker, and then incubated at 37 degrees C. for 2 hours. Following this, the plates were agitated at 80 degrees C. for 8 minutes.

### Real Time PCR

Two microlitre (μl) of cDNA were added to a master mix containing 0.5 μl of human GAPDH TaqMan Probe (4326317E), 0.5 μl human AGT, 2 μl nuclease-free water and 5 μl Lightcycler 480 probe master mix (Roche Cat #04887301001) per well in a 384 well plates (Roche cat #04887301001). Real time PCR was done in a LightCycler480 Real Time PCR system (Roche).

To calculate relative fold change, data were analyzed using the ΔΔCt method and normalized to assays performed with cells transfected with 10 nM AD-1955, or mock transfected cells. IC₅₀s were calculated using a 4 parameter fit model using XLFit and normalized to cells transfected with AD-1955 or mock-transfected. The sense and antisense sequences of AD-1955 are: sense: 5′-cuuAcGcuGAGuAcuucGAdTsdT-3′ and antisense 5′-UCGAAGuACUcAGCGuAAGdTsdT-3′. The results of the dual-luciferase assays of the agents listed in Tables 30 and 31 are provided in Table 36.

TABLE 36
Single Dose Reporter Screen for Human LEP in Hepa1-6 Cells

RLuc/FLuc
10 nM

| | % Message | |
|---|---|---|
| Duplex Name | Remaining | SD |
| AD-1947913.1 | 43.603 | 2.447 |
| AD-1947891.1 | 58.640 | 1.210 |
| AD-1947868.1 | 31.474 | 0.969 |
| AD-1947838.1 | 46.521 | 2.454 |
| AD-1947821.1 | 83.789 | 4.438 |
| AD-1947787.1 | 36.331 | 1.427 |
| AD-941419.1 | 47.050 | 2.614 |
| AD-1947724.1 | 30.677 | 0.743 |
| AD-1947747.1 | 34.652 | 1.368 |
| AD-1947713.1 | 40.581 | 2.760 |
| AD-1947689.1 | 43.628 | 1.151 |
| AD-1947661.1 | 37.525 | 1.138 |
| AD-1947643.1 | 38.039 | 3.247 |
| AD-1947625.1 | 38.507 | 1.343 |
| AD-1947609.1 | 72.801 | 1.141 |
| AD-1947581.1 | 79.702 | 1.776 |
| AD-1947537.1 | 37.364 | 2.772 |
| AD-1947522.1 | 35.942 | 0.882 |
| AD-1947507.1 | 36.951 | 0.626 |
| AD-1947489.1 | 46.426 | 1.774 |
| AD-1947459.1 | 81.536 | 2.751 |
| AD-1947440.1 | 61.008 | 1.210 |
| AD-1947423.1 | 53.159 | 1.375 |
| AD-1947396.1 | 56.099 | 1.389 |
| AD-1947381.1 | 61.241 | 3.458 |
| AD-1947366.1 | 50.593 | 2.097 |
| AD-1947343.1 | 42.641 | 1.768 |
| AD-1947328.1 | 43.813 | 1.044 |
| AD-1947313.1 | 40.401 | 2.279 |
| AD-1947288.1 | 46.571 | 2.579 |
| AD-1947266.1 | 46.348 | 0.904 |
| AD-1947249.1 | 53.461 | 2.807 |
| AD-1947233.1 | 81.878 | 3.064 |
| AD-1947204.1 | 41.342 | 0.563 |
| AD-1947186.1 | 35.394 | 1.249 |
| AD-1947158.1 | 41.903 | 1.667 |
| AD-1947129.1 | 45.153 | 1.855 |
| AD-1947113.1 | 42.953 | 2.207 |
| AD-1947105.1 | 52.166 | 2.202 |
| AD-1947083.1 | 42.067 | 2.123 |
| AD-1947068.1 | 41.200 | 0.885 |
| AD-1947049.1 | 50.618 | 2.650 |
| AD-1947028.1 | 51.813 | 2.991 |
| AD-1947013.1 | 61.578 | 2.017 |
| AD-1946998.1 | 60.723 | 0.755 |
| AD-1946984.1 | 49.100 | 0.725 |
| AD-1946965.1 | 47.300 | 1.205 |
| AD-1946937.1 | 58.866 | 1.661 |
| AD-1946910.1 | 31.610 | 1.426 |
| AD-1946895.1 | 23.692 | 0.569 |
| AD-1946879.1 | 65.108 | 2.337 |
| AD-1946860.1 | 65.307 | 2.102 |
| AD-1946845.1 | 79.606 | 0.818 |
| AD-1946831.1 | 33.578 | 0.286 |
| AD-1946816.1 | 72.449 | 3.972 |
| AD-1946775.1 | 50.696 | 1.851 |
| AD-1946745.1 | 54.226 | 2.705 |
| AD-1946729.1 | 48.246 | 1.728 |
| AD-1946720.1 | 60.826 | 1.540 |
| AD-1946696.1 | 29.266 | 1.250 |
| AD-1946681.1 | 38.420 | 1.814 |
| AD-1946675.1 | 47.619 | 1.832 |
| AD-1946654.1 | 39.292 | 1.833 |
| AD-1946634.1 | 45.094 | 2.684 |
| AD-1946617.1 | 38.242 | 1.563 |
| AD-1946602.1 | 51.676 | 1.117 |
| AD-1946579.1 | 41.965 | 3.569 |
| AD-1946555.1 | 44.655 | 3.361 |
| AD-1946540.1 | 50.290 | 2.364 |
| AD-1946511.1 | 36.491 | 4.415 |
| AD-1946496.1 | 35.628 | 0.542 |
| AD-1946472.1 | 40.298 | 0.781 |
| AD-1946455.1 | 15.510 | 0.230 |
| AD-1946438.1 | 28.956 | 0.710 |
| AD-1946422.1 | 19.287 | 0.467 |
| AD-1946402.1 | 29.636 | 1.066 |
| AD-1946383.1 | 8.690 | 0.152 |
| AD-1946366.1 | 23.871 | 1.745 |
| AD-1946336.1 | 6.380 | 0.297 |
| AD-1946314.1 | 38.183 | 1.490 |
| AD-1946298.1 | 35.136 | 0.953 |
| AD-1946273.1 | 7.719 | 0.650 |
| AD-1946258.1 | 6.801 | 0.428 |
| AD-1946238.1 | 10.955 | 0.470 |
| AD-1946222.1 | 13.814 | 1.065 |
| AD-1946207.1 | 29.775 | 1.684 |
| AD-1946189.1 | 21.264 | 1.258 |
| AD-1946159.1 | 28.747 | 2.396 |
| AD-1946129.1 | 32.971 | 1.969 |
| AD-1946112.1 | 48.966 | 2.897 |
| AD-1946092.1 | 23.863 | 1.513 |
| AD-1946076.1 | 40.021 | 4.141 |
| AD-1946060.1 | 14.895 | 0.784 |
| AD-1946044.1 | 48.890 | 1.195 |
| AD-1946025.1 | 37.091 | 0.805 |
| AD-1946002.1 | 10.544 | 0.417 |
| AD-1945987.1 | 16.883 | 0.930 |
| AD-1945966.1 | 15.010 | 0.264 |
| AD-1945947.1 | 7.818 | 0.374 |
| AD-1945935.1 | 11.128 | 0.309 |
| AD-1945919.1 | 16.097 | 0.745 |
| AD-1945903.1 | 41.216 | 1.166 |
| AD-1945888.1 | 80.942 | 4.615 |
| AD-1945878.1 | 8.663 | 0.727 |
| AD-1945851.1 | 12.559 | 1.161 |
| AD-1945823.1 | 57.799 | 1.818 |
| AD-1945801.1 | 9.824 | 0.799 |
| AD-1945785.1 | 11.348 | 0.382 |
| AD-1945774.1 | 9.080 | 0.648 |
| AD-1945754.1 | 19.588 | 1.001 |
| AD-1945739.1 | 19.742 | 1.110 |
| AD-1945724.1 | 25.956 | 0.638 |
| AD-1945708.1 | 76.824 | 4.360 |
| AD-1945658.1 | 59.073 | 2.903 |
| AD-1945639.1 | 33.564 | 1.280 |
| AD-1945624.1 | 38.002 | 1.267 |
| AD-1945611.1 | 82.959 | 2.823 |
| AD-1945579.1 | 12.399 | 0.311 |
| AD-1945558.1 | 40.552 | 2.467 |
| AD-1945537.1 | 77.963 | 1.551 |
| AD-1945518.1 | 9.156 | 0.630 |
| AD-1945500.1 | 20.272 | 1.212 |
| AD-1945483.1 | 15.547 | 0.946 |
| AD-1945454.1 | 13.943 | 0.452 |
| AD-1945439.1 | 10.560 | 0.408 |
| AD-1945422.1 | 13.636 | 1.277 |
| AD-1945401.1 | 11.282 | 0.322 |
| AD-1646889.1 | 10.741 | 0.392 |
| AD-1945386.1 | 9.855 | 0.659 |
| AD-1945372.1 | 21.061 | 0.495 |
| AD-1945347.1 | 15.416 | 0.808 |
| AD-1945327.1 | 6.105 | 0.196 |
| AD-1945308.1 | 17.009 | 0.767 |
| AD-1945298.1 | 7.306 | 0.188 |
| AD-1945272.1 | 89.310 | 1.201 |
| Positive control | 0.522 | 0.077 |
| (Rluc directed | | |
| siRNA) | | |

### Example 16: dsRNA Synthesis

siRNAs targeting the human phospholamban (PLN) gene (human: GenBank NM_002667.5, NCBI GeneID: 5350) and the human calcium/calmodulin dependent protein kinase II delta (CAMK2D) gene (human: GenBank NM_001321571.2, NCBI GeneID: 817) were designed using custom R and Python scripts.

The human PLN REFSEQ NM_002667.5 mRNA has a length of 2989 bases.

The human CAMK2D REFSEQ NM_001321571.2 mRNA has a length of 5785 bases.

siRNAs comprising a GalNAc conjugate targeting ligand were designed and synthesized as described above. siRNAs comprising an unsaturated C₂₂ hydrocarbon chain conjugated to the sense strand at position 6, counting from the 5′-end of the sense strand, were designed and synthesized as described above.

Detailed lists of the modified PLN sense and antisense strand nucleotide sequences comprising an unsaturated C₂₂ hydrocarbon chain conjugated to the sense strand at position 6, counting from the 5′-end of the sense strand, are shown in Table 38, and the corresponding unmodified PLN sense and antisense nucleotide sequences are shown in Table 37.

Detailed lists of the modified PLN sense and antisense strand nucleotide sequences comprising a GalNAc conjugate targeting ligand are shown in Table 40, and the corresponding unmodified PLN sense and antisense nucleotide sequences are shown in Table 39.

Detailed lists of the modified CAMK2D sense and antisense strand nucleotide sequences comprising an unsaturated C₂₂ hydrocarbon chain conjugated to the sense strand at position 6, counting from the 5′-end of the sense strand, are shown in Table 42, and the corresponding unmodified CAMK2D sense and antisense nucleotide sequences are shown in Table 41.

Detailed lists of the modified CAMK2D sense and antisense strand nucleotide sequences comprising a GalNAc conjugate targeting ligand are shown in Table 44, and the corresponding unmodified CAMK2D sense and antisense nucleotide sequences are shown in Table 43.

It is to be understood that, throughout the application, a duplex name without a decimal is equivalent to a duplex name with a decimal which merely references the batch number of the duplex. For example, AD-959917 is equivalent to AD-959917.1.

TABLE 37
Unmodified Sense and Antisense Strand Sequences of PLN dsRNA Agents
Comprising an Unsaturated C22 Hydrocarbon Chain Conjugated to
Position 6 on the Sense Strand, Counting from the 5′-end of the Sense Strand

| | | SEQ | Range in | | | SEQ | Range in | |
|---|---|---|---|---|---|---|---|---|
| Duplex | Sense Strand | ID | NM_00266.75 | | Antisense Strand | ID | NM_00266.75 | |
| Name | Sequence 5′ to 3′ | NO. | Start | End | Sequence 5′ to 3′ | NO. | Start | End |
| AD-2011169 | CUAUUUUUCUUCCUCUAUCAU | | 1888 | 1908 | AUGAUAGAGGAAGAAAAAUAGCU | | 1886 | 1908 |
| AD-2011170 | UAUCAAUUUCUGUCUCAUCUU | | 290 | 310 | AAGAUGAGACAGAAAUUGAUAAA | | 288 | 310 |
| AD-2011171 | AACAUAAAAGUCUUCAUUCUU | | 1694 | 1714 | AAGAAUGAAGACUUUUAUGUUGA | | 1692 | 1714 |
| AD-2011172 | UCUCAGUAAACAGAAAUAACU | | 2624 | 2644 | AGUUAUUUCUGUUUACUGAGAAA | | 2622 | 2644 |
| AD-2011173 | AUCUUAAUAUGUCUCUUGCUU | | 306 | 326 | AAGCAAGAGACAUAUUAAGAUGA | | 304 | 326 |
| AD-2011174 | CACAAAUUUCUAUCCCAAAUU | | 619 | 639 | AAUUUGGGAUAGAAAUUUGUGAG | | 617 | 639 |
| AD-2011175 | CUUCCAAUAACUCAUAAAACU | | 2034 | 2054 | AGUUUUAUGAGUUAUUGGAAGAU | | 2032 | 2054 |
| AD-2011176 | UAUCAGAAUCUACAUUCUAAU | | 890 | 910 | AUUAGAAUGUAGAUUCUGAUAGU | | 888 | 910 |
| AD-2011177 | UAUGAAUUCUCUCUCCAAAUU | | 1368 | 1388 | AAUUUGGAGAGAGAAUUCAUAUA | | 1366 | 1388 |
| AD-2011178 | UAACAACAGAAUCUAAUCUUU | | 1148 | 1168 | AAAGAUUAGAUUCUGUUGUUAGU | | 1146 | 1168 |
| AD-2011179 | CCUCACAUCUGUUAUCUUAUU | | 847 | 867 | AAUAAGAUAACAGAUGUGAGGAG | | 845 | 867 |
| AD-2011180 | CACUUAAAACUUCAGACUUCU | | 159 | 179 | AGAAGUCUGAAGUUUUAAGUGGU | | 157 | 179 |
| AD-2011181 | CAUUCUCAUUGUCUUCACAUU | | 1708 | 1728 | AAUGUGAAGACAAUGAGAAUGAA | | 1706 | 1728 |
| AD-2011182 | GACCAUUUCAGAACAUCUUCU | | 2018 | 2038 | AGAAGAUGUUCUGAAAUGGUCAG | | 2016 | 2038 |
| AD-2011183 | CCUUACUGAUAACAUAAACAU | | 1538 | 1558 | AUGUUUAUGUUAUCAGUAAGGUU | | 1536 | 1558 |
| AD-2011184 | UCUCUGAAGUUCUGCUACAAU | | 347 | 367 | AUUGUAGCAGAACUUCAGAGAAG | | 345 | 367 |
| AD-2011185 | CUACAGAAUCUAUUUAUCAAU | | 276 | 296 | AUUGAUAAAUAGAUUCUGUAGCU | | 274 | 296 |
| AD-2011186 | UAAGACUUCAGAAUGAUUUUU | | 1246 | 1266 | AAAAAUCAUUCUGAAGUCUUAAG | | 1244 | 1266 |
| AD-2011187 | AAGUAUUUUUCAGGUCUUCAU | | 769 | 789 | AUGAAGACCUGAAAAAUACUUAG | | 767 | 789 |
| AD-2011188 | ACUGUUGAUUUCCUCAACAUU | | 595 | 615 | AAUGUUGAGGAAAUCAACAGUUG | | 593 | 615 |
| AD-2011189 | CCUAUACUGCAUAAUCCAACU | | 2148 | 2168 | AGUUGGAUUAUGCAGUAUAGGUG | | 2146 | 2168 |
| AD-2011190 | CUGUUCUAAGACAUAUGAUCU | | 1069 | 1089 | AGAUCAUAUGUCUUAGAACAGAU | | 1067 | 1089 |
| AD-2011191 | UCUACUAUAGAAUAAGUUCUU | | 2834 | 2854 | AAGAACUUAUUCUAUAGUAGAAC | | 2832 | 2854 |
| AD-2011192 | AUGCUCAAAUAUGUUCUACUU | | 2820 | 2840 | AAGUAGAACAUAUUUGAGCAUUU | | 2818 | 2840 |
| AD-2011193 | UUCUAAAACAUGGUUACUAAU | | 2245 | 2265 | AUUAGUAACCAUGUUUUAGAAGA | | 2243 | 2265 |
| AD-2011194 | GGAAAAUAUAUUCACCAAACU | | 2076 | 2096 | AGUUUGGUGAAUAUAUUUUCCAG | | 2074 | 2096 |
| AD-2011195 | CAAAAUCUUAACUACCUAAUU | | 1498 | 1518 | AAUUAGGUAGUUAAGAUUUUGCG | | 1496 | 1518 |
| AD-2011196 | UUUUCAAUUUCUCCUCUGACU | | 2001 | 2021 | AGUCAGAGGAGAAAUUGAAAAUU | | 1999 | 2021 |
| AD-2011197 | UCUUUCUCUCGACCACUUAAU | | 146 | 166 | AUUAAGUGGUCGAGAGAAAGAUA | | 144 | 166 |
| AD-2011198 | GCUUACCAUACUAUAUCUUUU | | 1211 | 123 | AAAAGAUAUAGUAUGGUAAGCUA | | 1209 | 1231 |
| AD-2011199 | UUCACCAAGUAUCAAAGUAAU | | 785 | 805 | AUUACUUUGAUACUUGGUGAAGA | | 783 | 805 |
| AD-2011200 | CAUCAGCUUAAAAUCUGUCAU | | 389 | 409 | AUGACAGAUUUUAAGCUGAUGUG | | 387 | 409 |
| AD-2011201 | ACUAUUGACCAUAAACCUUAU | | 1523 | 1543 | AUAAGGUUUAUGGUCAAUAGUAG | | 1521 | 1543 |
| AD-2011202 | CUAUAUUCCUACAAUAAAGUU | | 1598 | 1618 | AACUUUAUUGUAGGAAUAUAGUG | | 1596 | 1618 |
| AD-2011203 | UACAAGCUGGAAAUUCCUAAU | | 2448 | 2468 | AUUAGGAAUUUCCAGCUUGUAGA | | 2446 | 2468 |
| AD-2011204 | UACUGAGCUAAAUUAUAGAUU | | 1942 | 1962 | AAUCUAUAAUUUAGCUCAGUAGA | | 1940 | 1962 |
| AD-2011205 | UGCCAACAAGUUCACUUCAUU | | 677 | 697 | AAUGAAGUGAACUUGUUGGCAGU | | 675 | 697 |
| AD-2011206 | CUACCUAAAAGAAGACAGUUU | | 99 | 119 | AAACUGUCUUCUUUUAGGUAGCC | | 97 | 119 |
| AD-2011207 | AGACAUGUUACUAAUAUAACU | | 2186 | 2206 | AGUUAUAUUAGUAACAUGUCUUC | | 2184 | 2206 |
| AD-2011208 | UUCAUACAACACAAUACUCUU | | 52 | 72 | AAGAGUAUUGUGUUGUAUGAAGU | | 50 | 72 |
| AD-2011209 | AUGCCACAUUAACAUCUUUUU | | 931 | 951 | AAAAAGAUGUUAAUGUGGCAUAG | | 929 | 951 |
| AD-2011210 | CACUGGUUUUAGUAAAUUACU | | 2511 | 2531 | AGUAAUUUACUAAAACCAGUGAG | | 2509 | 2531 |
| AD-2011211 | CUCGCUCAGCUAUAAGAAGAU | | 217 | 237 | AUCUUCUUAUAGCUGAGCGAGUG | | 215 | 237 |
| AD-2011212 | UCUUCAUUCUUUGAUAGAAAU | | 2656 | 2676 | AUUUCUAUCAAAGAAUGAAGAAC | | 2654 | 2676 |
| AD-2011213 | CAAGAUUAAGACUAAAACUUU | | 482 | 502 | AAAGUUUUAGUCUUAAUCUUGAC | | 480 | 502 |
| AD-2011214 | AAGCUAGAGAAAAUGUUAUUU | | 1618 | 1638 | AAAUAACAUUUUCUCUAGCUUAC | | 1616 | 1638 |
| AD-2011215 | AAACUUUGGUAAUUUAAGUUU | | 2092 | 2112 | AAACUUAAAUUACCAAAGUUUGG | | 2090 | 2112 |
| AD-2011216 | CAUAUCACUAAUAUACUAACU | | 1132 | 1152 | AGUUAGUAUAUUAGUGAUAUGAC | | 1130 | 1152 |
| AD-2011217 | UUGCUGAUCUGUAUCAUCGUU | | 321 | 341 | AACGAUGAUACAGAUCAGCAAGA | | 319 | 341 |
| AD-2011218 | AUCUGUUGGAUCUUGUAAACU | | 521 | 541 | AGUUUACAAGAUCCAACAGAUGA | | 519 | 541 |
| AD-2011219 | AUGUAGGUAAAUCAUAAAUCU | | 1050 | 1070 | AGAUUUAUGAUUUACCUACAUGU | | 1048 | 1070 |
| AD-2011220 | CUGAUUAGUCAUAUUCCUUUU | | 2578 | 2598 | AAAAGGAAUAUGACUAAUCAGUU | | 2576 | 2598 |
| AD-2011221 | CAUCUAUUACAUCUACAGCUU | | 1432 | 1452 | AAGCUGUAGAUGUAAUAGAUGGG | | 1430 | 1452 |
| AD-2011222 | AAAAUAGUUUACACCUAUACU | | 2135 | 2155 | AGUAUAGGUGUAAACUAUUUUAG | | 2133 | 2155 |
| AD-2011223 | GGAGACACUAUUAAAUUUUCU | | 2329 | 2349 | AGAAAAUUUAAUAGUGUCUCCUU | | 2327 | 2349 |
| AD-2011224 | AGUAGAAAGCUUAUAAACAAU | | 2473 | 2493 | AUUGUUUAUAAGCUUUCUACUUG | | 2471 | 2493 |
| AD-2011225 | UCUUACACAAGUGUUGCUAAU | | 2297 | 2317 | AUUAGCAACACUUGUGUAAGAAA | | 2295 | 2317 |
| AD-2011226 | AACUAUUUGUAGUAACUAUCU | | 874 | 894 | AGAUAGUUACUACAAAUAGUUCU | | 872 | 894 |
| AD-2011227 | UUACUAAAAGAAUAUGUAACU | | 2258 | 2278 | AGUUACAUAUUCUUUUAGUAACC | | 2256 | 2278 |
| AD-2011228 | UCAAAAUAGUCCACUGACUCU | | 828 | 848 | AGAGUCAGUGGACUAUUUUGAAU | | 826 | 848 |
| AD-2011229 | ACAGGAAAACAAUAUUGUAUU | | 419 | 439 | AAUACAAUAUUGUUUUCCUGUCU | | 417 | 439 |
| AD-2011230 | GUGUAUAAAAUGCAACUGUUU | | 581 | 601 | AAACAGUUGCAUUUUAUACACUU | | 579 | 601 |
| AD-2011231 | CUAUACUGUGAUGAUCACAGU | | 70 | 90 | ACUGUGAUCAUCACAGUAUAGAG | | 68 | 90 |
| AD-2011232 | GCCAUACUCUUACAUAAUAAU | | 984 | 1004 | AUUAUUAUGUAAGAGUAUGGCCU | | 982 | 1004 |
| AD-2011233 | GAAUCACAGAAUUCUAGUACU | | 1030 | 1050 | AGUACUAGAAUUCUGUGAUUCUU | | 1028 | 1050 |
| AD-2011234 | ACACAUAUUUUGCGUGUUAUU | | 1566 | 1586 | AAUAACACGCAAAAUAUGUGUUA | | 1564 | 1586 |
| AD-2011235 | UUGAUUACACUGUUUGUUACU | | 2596 | 2616 | AGUAACAAACAGUGUAAUCAAAG | | 2594 | 2616 |
| AD-2011236 | CCAGCUAUGCUAUUUAUAAUU | | 1962 | 1982 | AAUUAUAAAUAGCAUAGCUGGAU | | 1960 | 1982 |
| AD-2011237 | CAACUGAAGUAAAAUUGAGUU | | 2053 | 2073 | AACUCAAUUUUACUUCAGUUGUU | | 2051 | 2073 |
| AD-2011238 | GUGUAAUUAACCAUAUCUUCU | | 2228 | 2248 | AGAAGAUAUGGUUAAUUACACAU | | 2226 | 2248 |
| AD-2011239 | UCUAUCAACCAAAUGGUAAGU | | 1901 | 1921 | ACUUACCAUUUGGUUGAUAGAGG | | 1899 | 1921 |
| AD-2011240 | UGAGGAUUACAGAAUACUAUU | | 2692 | 2712 | AAUAGUAUUCUGUAAUCCUCACA | | 2690 | 2712 |
| AD-2011241 | GUGGAAAAUUUGAACUGAUUU | | 2564 | 2584 | AAAUCAGUUCAAAUUUUCCACUU | | 2562 | 2584 |
| AD-2011242 | AUGGAGAAAGUCCAAUACCUU | | 195 | 215 | AAGGUAUUGGACUUUCUCCAUGA | | 193 | 215 |
| AD-2011243 | AAAUCAGAAUCACUAUAUUAU | | 2947 | 2967 | AUAAUAUAGUGAUUCUGAUUUGC | | 2945 | 2967 |
| AD-2011244 | CUUCCAUUCCAGCCUAACAUU | | 1275 | 1295 | AAUGUUAGGCUGGAAUGGAAGAC | | 1273 | 1295 |
| AD-2011245 | AAUGAAGUGUCAUUAUUCAAU | | 812 | 832 | AUUGAAUAAUGACACUUCAUUUG | | 810 | 832 |
| AD-2011246 | CUUCAUCAUAAAGUGUAAAGU | | 2754 | 2774 | ACUUUACACUUUAUGAUGAAGAA | | 2752 | 2774 |
| AD-2011247 | CCAAAUCUUUUCUGAAGAUGU | | 633 | 653 | ACAUCUUCAGAAAAGAUUUGGGA | | 631 | 653 |
| AD-2011248 | UUUGGAAUCAUGAAACCUUAU | | 1228 | 1248 | AUAAGGUUUCAUGAUUCCAAAGA | | 1226 | 1248 |
| AD-2011249 | AAACACCCGUAAGACUUCAUU | | 37 | 57 | AAUGAAGUCUUACGGGUGUUUAG | | 35 | 57 |
| AD-2011250 | AACUCAAUAGUGAAGGAGACU | | 2315 | 2335 | AGUCUCCUUCACUAUUGAGUUAG | | 2313 | 2335 |
| AD-2011251 | UCUUCAUUUAAGGCACUGUAU | | 1164 | 1184 | AUACAGUGCCUUAAAUGAAGAUU | | 1162 | 1184 |
| AD-2011252 | GGUCUUGCAGUCUUGUCUUAU | | 1773 | 1793 | AUAAGACAAGACUGCAAGACCAA | | 1771 | 1793 |
| AD-2011253 | UAUCUGAGCUAGAGUUACCUU | | 1190 | 1210 | AAGGUAACUCUAGCUCAGAUAAU | | 1188 | 1210 |
| AD-2011254 | AACUGUAAUAGGAUAUAGCUU | | 1870 | 1890 | AAGCUAUAUCCUAUUACAGUUGA | | 1868 | 1890 |
| AD-2011255 | AUGAAAAGGGCUUUAUUUUCU | | 541 | 561 | AGAAAAUAAAGCCCUUUUCAUGU | | 539 | 561 |
| AD-2011256 | CAGAAAGUAUCCCUAGUCUUU | | 2536 | 2556 | AAAGACUAGGGAUACUUUCUGUA | | 2534 | 2556 |
| AD-2011257 | UGCCAGCUUUUUAUCUUUCUU | | 133 | 153 | AAGAAAGAUAAAAAGCUGGCAGC | | 131 | 153 |
| AD-2011258 | CUUUUGAGGUGAAUAUAAUUU | | 718 | 738 | AAAUUAUAUUCACCUCAAAAGAG | | 716 | 738 |
| AD-2011259 | UCAGGGAUAUCUGAAGAACAU | | 2395 | 2415 | AUGUUCUUCAGAUAUCCCUGAAC | | 2393 | 2415 |
| AD-2011260 | CAACAAGCACGUCAAAAGCUU | | 258 | 278 | AAGCUUUUGACGUGCUUGUUGAG | | 256 | 278 |
| AD-2011261 | AUUGUAUAACAGACCACUUCU | | 432 | 452 | AGAAGUGGUCUGUUAUACAAUAU | | 430 | 452 |
| AD-2011262 | UUAAUGUUGCCUUUUAUAUUU | | 2894 | 2914 | AAAUAUAAAAGGCAACAUUAAGC | | 2892 | 2914 |
| AD-2011263 | AUUGACCUUGGUUUCUUACAU | | 2284 | 2304 | AUGUAAGAAACCAAGGUCAAUAU | | 2282 | 2304 |
| AD-2011264 | UAUGAUCAACAGAUGAGAACU | | 1082 | 1102 | AGUUCUCAUCUGUUGAUCAUAUG | | 1080 | 1102 |
| AD-2011265 | GCAGAGAUUUCUUAAGUGACU | | 2420 | 2440 | AGUCACUUAAGAAAUCUCUGCCC | | 2418 | 2440 |
| AD-2011266 | GAAGAGUUUCUUUGUGAAAAU | | 459 | 479 | AUUUUCACAAAGAAACUCUUCUA | | 457 | 479 |
| AD-2011267 | CAACCAUUGAAAUGCCUCAAU | | 241 | 261 | AUUGAGGCAUUUCAAUGGUUGAG | | 239 | 261 |
| AD-2011268 | AUCUAUUUUGCAGUCCACUCU | | 1922 | 1942 | AGAGUGGACUGCAAAAUAGAUGC | | 1920 | 1942 |
| AD-2011269 | GUUACCAUAUGUAUUCAUCUU | | 505 | 525 | AAGAUGAAUACAUAUGGUAACAA | | 503 | 525 |
| AD-2011270 | UUCUUAUCUUAAUUUACAGGU | | 2850 | 2870 | ACCUGUAAAUUAAGAUAAGAACU | | 2848 | 2870 |
| AD-2011271 | ACAGUUAUCUCAUAUUUGGCU | | 113 | 133 | AGCCAAAUAUGAGAUAACUGUCU | | 111 | 133 |
| AD-2011272 | AACAAUUUUAAUUUCAGUUGU | | 2165 | 2185 | ACAACUGAAAUUAAAAUUGUUGG | | 2163 | 2185 |
| AD-2011273 | GGCAAGGAAAAUAAAAGAUUU | | 1303 | 1323 | AAAUCUUUUAUUUUCCUUGCCUG | | 1301 | 1323 |
| AD-2011274 | UCAUCGUGAUGCUUCUCUGAU | | 334 | 354 | AUCAGAGAAGCAUCACGAUGAUA | | 332 | 354 |
| AD-2011275 | AAGAUGAAGAGUUUAGUUUUU | | 647 | 667 | AAAAACUAAACUCUUCAUCUUCA | | 645 | 667 |
| AD-2011276 | CACUGUAGUGAAUUAUCUGAU | | 1177 | 1197 | AUCAGAUAAUUCACUACAGUGCC | | 1175 | 1197 |
| AD-2011277 | CUACAACCUCUAGAUCUGCAU | | 361 | 381 | AUGCAGAUCUAGAGGUUGUAGCA | | 359 | 381 |
| AD-2011278 | AUGAGAAUCAAGUAUGGAAAU | | 958 | 978 | AUUUCCAUACUUGAUUCUCAUCA | | 956 | 978 |
| AD-2011279 | UUGUUCAAGGGUCAACUGUAU | | 1857 | 1877 | AUACAGUUGACCCUUGAACAACA | | 1855 | 1877 |
| AD-2011280 | GAACCCAUGAGAGAUACUAGU | | 2350 | 2370 | ACUAGUAUCUCUCAUGGGUUCAG | | 2348 | 2370 |
| AD-2011281 | AUGUGACAGUGAGAUUAGUCU | | 1113 | 1133 | AGACUAAUCUCACUGUCACAUAU | | 1111 | 1133 |
| AD-2011282 | CUAUUCAUUAAAUGGAAGUGU | | 1670 | 1690 | ACACUUCCAUUUAAUGAAUAGUA | | 1668 | 1690 |
| AD-2011283 | UCUUUUAAAGUUGAUGAGAAU | | 945 | 965 | AUUCUCAUCAACUUUAAAAGAUG | | 943 | 965 |
| AD-2011284 | GGUGACAGAGUCAGAAAACUU | | 9 | 29 | AAGUUUUCUGACUCUGUCACCCA | | 7 | 29 |
| AD-2011285 | AAACAACAGGUGAUACACUCU | | 2487 | 2507 | AGAGUGUAUCACCUGUUGUUUAU | | 2485 | 2507 |
| AD-2011286 | CCACUUCCUGAGUAGAAGAGU | | 445 | 465 | ACUCUUCUACUCAGGAAGUGGUC | | 443 | 465 |
| AD-2011287 | AAAUCUUAUUCUGUGAGGAUU | | 2679 | 2699 | AAUCCUCACAGAAUAAGAUUUUA | | 2677 | 2699 |
| AD-2011288 | AGUGGAAAGUGUUUGGUUCAU | | 2378 | 2398 | AUGAACCAAACACUUUCCACUCC | | 2376 | 2398 |
| AD-2011289 | UUGUAUUUUUUCUAUGCCACU | | 918 | 938 | AGUGGCAUAGAAAAAAUACAAUU | | 916 | 938 |
| AD-2011290 | GUGUUAUAUGUAUUAUACACU | | 1579 | 1599 | AGUGUAUAAUACAUAUAACACGC | | 1577 | 1599 |
| AD-2011291 | AAAGAGUAGAGGAUGUGUAAU | | 2214 | 2234 | AUUACACAUCCUCUACUCUUUUA | | 2212 | 2234 |
| AD-2011292 | AGUUUUAAAACUGCACUGCCU | | 661 | 681 | AGGCAGUGCAGUUUUAAAACUAA | | 659 | 681 |
| AD-2011293 | AAGAUUUCCAGUGACAGAAAU | | 1317 | 1337 | AUUUCUGUCACUGGAAAUCUUUU | | 1315 | 1337 |
| AD-2011294 | ACUGGUGGUUAAUAUGUGACU | | 1100 | 1120 | AGUCACAUAUUAACCACCAGUUC | | 1098 | 1120 |
| AD-2011295 | UUGCAAUUCAAGCCCUUGUUU | | 1839 | 1859 | AAACAAGGGCUUGAAUUGCAAGG | | 1837 | 1859 |
| AD-2011296 | AAGUGACGCCUCAUCUACAAU | | 2433 | 2453 | AUUGUAGAUGAGGCGUCACUUAA | | 2431 | 2453 |
| AD-2011297 | AUUUUGCAGGUUGUCUUCCAU | | 1261 | 1281 | AUGGAAGACAACCUGCAAAAUCA | | 1259 | 1281 |
| AD-2011298 | AUGUAACAUCAAUAUUGACCU | | 2271 | 2291 | AGGUCAAUAUUGAUGUUACAUAU | | 2269 | 2291 |
| AD-2011299 | AAAGAAUAUUCAUGUAUAAGU | | 1812 | 1832 | ACUUAUACAUGAAUAUUCUUUCC | | 1810 | 1832 |
| AD-2011300 | AAGUAAUAACACAAAUGAAGU | | 799 | 819 | ACUUCAUUUGUGUUAUUACUUUG | | 797 | 819 |
| AD-2011301 | UACAGCUGACCCUUGAACAUU | | 1445 | 1465 | AAUGUUCAAGGGUCAGCUGUAGA | | 1443 | 1465 |
| AD-2011302 | AUGGAAAAGUAAGGCCAUACU | | 971 | 991 | AGUAUGGCCUUACUUUUCCAUAC | | 969 | 991 |
| AD-2011303 | GAACUUGUUGGCCCAUCUAUU | | 1419 | 1439 | AAUAGAUGGGCCAACAAGUUCAU | | 1417 | 1439 |

TABLE 38
Modified Sense and Antisense Strand Sequences of PLN dsRNA Agents
Comprising an Unsaturated C22 Hydrocarbon Chain Conjugated
to Position 6 on the Sense Strand, Counting from the 5′-end of the Sense Strand

| Duplex Name | Sense Strand Sequence 5′ to 3′ | SEQ ID NO. | Antisense Strand Sequence 5′ to 3′ | SEQ ID NO. | mRNA target sequence | SEQ ID NO. |
|---|---|---|---|---|---|---|
| AD-2011169 | csusauu(Uda)UfuCfUfUfccucuaucauL96 | | asUfsgauAfgAfGfgaagAfaAfaauagscsu | | AGCUAUUUUUCUUCCUCUAUCAA | |
| AD-2011170 | usasuca(Ada)UfuUfCfUfgucucaucuuL96 | | asAfsgauGfaGfAfcagaAfaUfugauasasa | | UUUAUCAAUUUCUGUCUCAUCUU | |
| AD-2011171 | asascau(Ada)AfaAfGfUfcuucauucuuL96 | | asAfsgaaUfgAfAfgacuUfuUfauguusgsa | | UCAACAUAAAAGUCUUCAUUCUC | |
| AD-2011172 | uscsuca(Gda)UfaAfAfCfagaaauaacuL96 | | asGfsuuaUfuUfCfuguuUfaCfugagasasa | | UUUCUCAGUAAACAGAAAUAACU | |
| AD-2011173 | asuscuu(Ada)AfuAfUfGfucucuugcuuL96 | | asAfsgcaAfgAfGfacauAfuUfaagausgsa | | UCAUCUUAAUAUGUCUCUUGCUG | |
| AD-2011174 | csascaa(Ada)UfuUfCfUfaucccaaauuL96 | | asAfsuuuGfgGfAfuagaAfaUfuugugsasg | | CUCACAAAUUUCUAUCCCAAAUC | |
| AD-2011175 | csusucc(Ada)AfuAfAfCfucauaaaacuL96 | | asGfsuuuUfaUfGfaguuAfuUfggaagsasu | | AUCUUCCAAUAACUCAUAAAACA | |
| AD-2011176 | usasuca(Gda)AfaUfCfUfacauucuaauL96 | | asUfsuagAfaUfGfuagaUfuCfugauasgsu | | ACUAUCAGAAUCUACAUUCUAAA | |
| AD-2011177 | usasuga(Ada)UfuCfUfCfucuccaaauuL96 | | asAfsuuuGfgAfGfagagAfaUfucauasusa | | UAUAUGAAUUCUCUCUCCAAAUA | |
| AD-2011178 | usasaca(Ada)CfaGfAfAfucuaaucuuuL96 | | asAfsagaUfuAfGfauucUfgUfuguuasgsu | | ACUAACAACAGAAUCUAAUCUUC | |
| AD-2011179 | cscsuca(Cda)AfuCfUfGfuuaucuuauuL96 | | asAfsuaaGfaUfAfacagAfuGfugaggsasg | | CUCCUCACAUCUGUUAUCUUAUU | |
| AD-2011180 | csascuu(Ada)AfaAfCfUfucagacuucuL96 | | asGfsaagUfcUfGfaaguUfuUfaagugsgsu | | ACCACUUAAAACUUCAGACUUCC | |
| AD-2011181 | csasuuc(Uda)CfaUfUfGfucuucacauuL96 | | | | | |
| AD-2011182 | gsascca(Uda)UfuCfAfGfaacaucuucuL96 | | asGfsaagAfuGfUfucugAfaAfuggucsasg | | CUGACCAUUUCAGAACAUCUUCC | |
| AD-2011183 | cscsuua(Cda)UfgAfUfAfacauaaacauL96 | | asUfsguuUfaUfGfuuauCfaGfuaaggsusu | | AACCUUACUGAUAACAUAAACAG | |
| AD-2011184 | uscsucu(Gda)AfaGfUfUfcugcuacaauL96 | | asUfsuguAfgCfAfgaacUfuCfagagasasg | | CUUCUCUGAAGUUCUGCUACAAC | |
| AD-2011185 | csusaca(Gda)AfaUfCfUfauuuaucaauL96 | | asUfsugaUfaAfAfuagaUfuCfuguagscsu | | AGCUACAGAAUCUAUUUAUCAAU | |
| AD-2011186 | usasaga(Cda)UfuCfAfGfaaugauuuuuL96 | | asAfsaaaUfcAfUfucugAfaGfucuuasasg | | CUUAAGACUUCAGAAUGAUUUUG | |
| AD-2011187 | asasgua(Uda)UfuUfUfCfaggucuucauL96 | | asUfsgaaGfaCfCfugaaAfaAfuacuusasg | | CUAAGUAUUUUUCAGGUCUUCAC | |
| AD-2011188 | ascsugu(Uda)GfaUfUfUfccucaacauuL96 | | asAfsuguUfgAfGfgaaaUfcAfacagususg | | CAACUGUUGAUUUCCUCAACAUG | |
| AD-2011189 | cscsuau(Ada)CfuGfCfAfuaauccaacuL96 | | asGfsuugGfaUfUfaugcAfgUfauaggsusg | | CACCUAUACUGCAUAAUCCAACA | |
| AD-2011190 | csusguu(Cda)UfaAfGfAfcauaugaucuL96 | | asGfsaucAfuAfUfgucuUfaGfaacagsasu | | AUCUGUUCUAAGACAUAUGAUCA | |
| AD-2011191 | uscsuac(Uda)AfuAfGfAfauaaguucuuL96 | | asAfsgaaCfuUfAfuucuAfuAfguagasasc | | GUUCUACUAUAGAAUAAGUUCUU | |
| AD-2011192 | asusgcu(Cda)AfaAfUfAfuguucuacuuL96 | | asAfsguaGfaAfCfauauUfuGfagcaususu | | AAAUGCUCAAAUAUGUUCUACUA | |
| AD-2011193 | ususcua(Ada)AfaCfAfUfgguuacuaauL96 | | asUfsuagUfaAfCfcaugUfuUfuagaasgsa | | UCUUCUAAAACAUGGUUACUAAA | |
| AD-2011194 | gsgsaaa(Ada)UfaUfAfUfucaccaaacuL96 | | asGfsuuuGfgUfGfaauaUfaUfuuuccsasg | | CUGGAAAAUAUAUUCACCAAACU | |
| AD-2011195 | csasaaa(Uda)CfuUfAfAfcuaccuaauuL96 | | asAfsuuaGfgUfAfguuaAfgAfuuuugscsg | | CGCAAAAUCUUAACUACCUAAUA | |
| AD-2011196 | ususuuc(Ada)AfuUfUfCfuccucugacuL96 | | asGfsucaGfaGfGfagaaAfuUfgaaaasusu | | AAUUUUCAAUUUCUCCUCUGACC | |
| AD-2011197 | uscsuuu(Cda)UfcUfCfGfaccacuuaauL96 | | asUfsuaaGfuGfGfucgaGfaGfaaagasusa | | UAUCUUUCUCUCGACCACUUAAA | |
| AD-2011198 | gscsuua(Cda)CfaUfAfCfuauaucuuuuL96 | | asAfsaagAfuAfUfaguaUfgGfuaagcsusa | | UAGCUUACCAUACUAUAUCUUUG | |
| AD-2011199 | ususcac(Cda)AfaGfUfAfucaaaguaauL96 | | asUfsuacUfuUfGfauacUfuGfgugaasgsa | | UCUUCACCAAGUAUCAAAGUAAU | |
| AD-2011200 | csasuca(Gda)CfuUfAfAfaaucugucauL96 | | asUfsgacAfgAfUfuuuaAfgCfugaugsusg | | CACAUCAGCUUAAAAUCUGUCAU | |
| AD-2011201 | ascsuau(Uda)GfaCfCfAfuaaaccuuauL96 | | asUfsaagGfuUfUfauggUfcAfauagusasg | | CUACUAUUGACCAUAAACCUUAC | |
| AD-2011202 | csusaua(Uda)UfcCfUfAfcaauaaaguuL96 | | asAfscuuUfaUfUfguagGfaAfuauagsusg | | CACUAUAUUCCUACAAUAAAGUA | |
| AD-2011203 | usascaa(Gda)CfuGfGfAfaauuccuaauL96 | | asUfsuagGfaAfUfuuccAfgCfuuguasgsa | | UCUACAAGCUGGAAAUUCCUAAA | |
| AD-2011204 | usascug(Ada)GfcUfAfAfauuauagauuL96 | | asAfsucuAfuAfAfuuuaGfcUfcaguasgsa | | UCUACUGAGCUAAAUUAUAGAUC | |
| AD-2011205 | usgscca(Ada)CfaAfGfUfucacuucauuL96 | | asAfsugaAfgUfGfaacuUfgUfuggcasgsu | | ACUGCCAACAAGUUCACUUCAUA | |
| AD-2011206 | csusacc(Uda)AfaAfAfGfaagacaguuuL96 | | asAfsacuGfuCfUfucuuUfuAfgguagscsc | | GGCUACCUAAAAGAAGACAGUUA | |
| AD-2011207 | asgsaca(Uda)GfuUfAfCfuaauauaacuL96 | | asGfsuuaUfaUfUfaguaAfcAfugucususc | | GAAGACAUGUUACUAAUAUAACU | |
| AD-2011208 | ususcau(Ada)CfaAfCfAfcaauacucuuL96 | | asAfsgagUfaUfUfguguUfgUfaugaasgsu | | ACUUCAUACAACACAAUACUCUA | |
| AD-2011209 | asusgcc(Ada)CfaUfUfAfacaucuuuuuL96 | | asAfsaaaGfaUfGfuuaaUfgUfggcausasg | | CUAUGCCACAUUAACAUCUUUUA | |
| AD-2011210 | csascug(Gda)UfuUfUfAfguaaauuacuL96 | | asGfsuaaUfuUfAfcuaaAfaCfcagugsasg | | CUCACUGGUUUUAGUAAAUUACC | |
| AD-2011211 | csuscgc(Uda)CfaGfCfUfauaagaagauL96 | | asUfscuuCfuUfAfuagcUfgAfgcgagsusg | | CACUCGCUCAGCUAUAAGAAGAG | |
| AD-2011212 | uscsuuc(Ada)UfuCfUfUfugauagaaauL96 | | asUfsuucUfaUfCfaaagAfaUfgaagasasc | | GUUCUUCAUUCUUUGAUAGAAAU | |
| AD-2011213 | csasaga(Uda)UfaAfGfAfcuaaaacuuuL96 | | asAfsaguUfuUfAfgucuUfaAfucuugsasc | | GUCAAGAUUAAGACUAAAACUUA | |
| AD-2011214 | asasgcu(Ada)GfaGfAfAfaauguuauuuL96 | | asAfsauaAfcAfUfuuucUfcUfagcuusasc | | GUAAGCUAGAGAAAAUGUUAUUU | |
| AD-2011215 | asasacu(Uda)UfgGfUfAfauuuaaguuuL96 | | asAfsacuUfaAfAfuuacCfaAfaguuusgsg | | CCAAACUUUGGUAAUUUAAGUUG | |
| AD-2011216 | csasuau(Cda)AfcUfAfAfuauacuaacuL96 | | asGfsuuaGfuAfUfauuaGfuGfauaugsasc | | GUCAUAUCACUAAUAUACUAACA | |
| AD-2011217 | ususgcu(Gda)AfuCfUfGfuaucaucguuL96 | | asAfscgaUfgAfUfacagAfuCfagcaasgsa | | UCUUGCUGAUCUGUAUCAUCGUG | |
| AD-2011218 | asuscug(Uda)UfgGfAfUfcuuguaaacuL96 | | asGfsuuuAfcAfAfgaucCfaAfcagausgsa | | UCAUCUGUUGGAUCUUGUAAACA | |
| AD-2011219 | asusgua(Gda)GfuAfAfAfucauaaaucuL96 | | asGfsauuUfaUfGfauuuAfcCfuacausgsu | | ACAUGUAGGUAAAUCAUAAAUCU | |
| AD-2011220 | csusgau(Uda)AfgUfCfAfuauuccuuuuL96 | | asAfsaagGfaAfUfaugaCfuAfaucagsusu | | AACUGAUUAGUCAUAUUCCUUUG | |
| AD-2011221 | csasucu(Ada)UfuAfCfAfucuacagcuuL96 | | asAfsgcuGfuAfGfauguAfaUfagaugsgsg | | CCCAUCUAUUACAUCUACAGCUG | |
| AD-2011222 | asasaau(Ada)GfuUfUfAfcaccuauacuL96 | | asGfsuauAfgGfUfguaaAfcUfauuuusasg | | CUAAAAUAGUUUACACCUAUACU | |
| AD-2011223 | gsgsaga(Cda)AfcUfAfUfuaaauuuucuL96 | | asGfsaaaAfuUfUfaauaGfuGfucuccsusu | | AAGGAGACACUAUUAAAUUUUCU | |
| AD-2011224 | asgsuag(Ada)AfaGfCfUfuauaaacaauL96 | | asUfsuguUfuAfUfaagcUfuUfcuacususg | | CAAGUAGAAAGCUUAUAAACAAC | |
| AD-2011225 | uscsuua(Cda)AfcAfAfGfuguugcuaauL96 | | asUfsuagCfaAfCfacuuGfuGfuaagasasa | | UUUCUUACACAAGUGUUGCUAAC | |
| AD-2011226 | asascua(Uda)UfuGfUfAfguaacuaucuL96 | | asGfsauaGfuUfAfcuacAfaAfuaguuscsu | | AGAACUAUUUGUAGUAACUAUCA | |
| AD-2011227 | ususacu(Ada)AfaAfGfAfauauguaacuL96 | | asGfsuuaCfaUfAfuucuUfuUfaguaascsc | | GGUUACUAAAAGAAUAUGUAACA | |
| AD-2011228 | uscsaaa(Ada)UfaGfUfCfcacugacucuL96 | | asGfsaguCfaGfUfggacUfaUfuuugasasu | | AUUCAAAAUAGUCCACUGACUCC | |
| AD-2011229 | ascsagg(Ada)AfaAfCfAfauauuguauuL96 | | asAfsuacAfaUfAfuuguUfuUfccuguscsu | | AGACAGGAAAACAAUAUUGUAUA | |
| AD-2011230 | gsusgua(Uda)AfaAfAfUfgcaacuguuuL96 | | asAfsacaGfuUfGfcauuUfuAfuacacsusu | | AAGUGUAUAAAAUGCAACUGUUG | |
| AD-2011231 | csusaua(Cda)UfgUfGfAfugaucacaguL96 | | asCfsuguGfaUfCfaucaCfaGfuauagsasg | | CUCUAUACUGUGAUGAUCACAGC | |
| AD-2011232 | gscscau(Ada)CfuCfUfUfacauaauaauL96 | | asUfsuauUfaUfGfuaagAfgUfauggcscsu | | AGGCCAUACUCUUACAUAAUAAA | |
| AD-2011233 | gsasauc(Ada)CfaGfAfAfuucuaguacuL96 | | asGfsuacUfaGfAfauucUfgUfgauucsusu | | AAGAAUCACAGAAUUCUAGUACA | |
| AD-2011234 | ascsaca(Uda)AfuUfUfUfgcguguuauuL96 | | asAfsuaaCfaCfGfcaaaAfuAfugugususa | | UAACACAUAUUUUGCGUGUUAUA | |
| AD-2011235 | ususgau(Uda)AfcAfCfUfguuuguuacuL96 | | asGfsuaaCfaAfAfcaguGfuAfaucaasasg | | CUUUGAUUACACUGUUUGUUACA | |
| AD-2011236 | cscsagc(Uda)AfuGfCfUfauuuauaauuL96 | | asAfsuuaUfaAfAfuagcAfuAfgcuggsasu | | AUCCAGCUAUGCUAUUUAUAAUU | |
| AD-2011237 | csasacu(Gda)AfaGfUfAfaaauugaguuL96 | | asAfscucAfaUfUfuuacUfuCfaguugsusu | | AACAACUGAAGUAAAAUUGAGUG | |
| AD-2011238 | gsusgua(Ada)UfuAfAfCfcauaucuucuL96 | | asGfsaagAfuAfUfgguuAfaUfuacacsasu | | AUGUGUAAUUAACCAUAUCUUCU | |
| AD-2011239 | uscsuau(Cda)AfaCfCfAfaaugguaaguL96 | | asCfsuuaCfcAfUfuuggUfuGfauagasgsg | | CCUCUAUCAACCAAAUGGUAAGC | |
| AD-2011240 | usgsagg(Ada)UfuAfCfAfgaauacuauuL96 | | asAfsuagUfaUfUfcuguAfaUfccucascsa | | UGUGAGGAUUACAGAAUACUAUA | |
| AD-2011241 | gsusgga(Ada)AfaUfUfUfgaacugauuuL96 | | asAfsaucAfgUfUfcaaaUfuUfuccacsusu | | AAGUGGAAAAUUUGAACUGAUUA | |
| AD-2011242 | asusgga(Gda)AfaAfGfUfccaauaccuuL96 | | asAfsgguAfuUfGfgacuUfuCfuccausgsa | | UCAUGGAGAAAGUCCAAUACCUC | |
| AD-2011243 | asasauc(Ada)GfaAfUfCfacuauauuauL96 | | asUfsaauAfuAfGfugauUfcUfgauuusgsc | | GCAAAUCAGAAUCACUAUAUUAA | |
| AD-2011244 | csusucc(Ada)UfuCfCfAfgccuaacauuL96 | | asAfsuguUfaGfGfcuggAfaUfggaagsasc | | GUCUUCCAUUCCAGCCUAACAUC | |
| AD-2011245 | asasuga(Ada)GfuGfUfCfauuauucaauL96 | | asUfsugaAfuAfAfugacAfcUfucauususg | | CAAAUGAAGUGUCAUUAUUCAAA | |
| AD-2011246 | csusuca(Uda)CfaUfAfAfaguguaaaguL96 | | asCfsuuuAfcAfCfuuuaUfgAfugaagsasa | | UUCUUCAUCAUAAAGUGUAAAGA | |
| AD-2011247 | cscsaaa(Uda)CfuUfUfUfcugaagauguL96 | | asCfsaucUfuCfAfgaaaAfgAfuuuggsgsa | | UCCCAAAUCUUUUCUGAAGAUGA | |
| AD-2011248 | ususugg(Ada)AfuCfAfUfgaaaccuuauL96 | | asUfsaagGfuUfUfcaugAfuUfccaaasgsa | | UCUUUGGAAUCAUGAAACCUUAA | |
| AD-2011249 | asasaca(Cda)CfcGfUfAfagacuucauuL96 | | asAfsugaAfgUfCfuuacGfgGfuguuusasg | | CUAAACACCCGUAAGACUUCAUA | |
| AD-2011250 | asascuc(Ada)AfuAfGfUfgaaggagacuL96 | | asGfsucuCfcUfUfcacuAfuUfgaguusasg | | CUAACUCAAUAGUGAAGGAGACA | |
| AD-2011251 | uscsuuc(Ada)UfuUfAfAfggcacuguauL96 | | asUfsacaGfuGfCfcuuaAfaUfgaagasusu | | AAUCUUCAUUUAAGGCACUGUAG | |
| AD-2011252 | gsgsucu(Uda)GfcAfGfUfcuugucuuauL96 | | asUfsaagAfcAfAfgacuGfcAfagaccsasa | | UUGGUCUUGCAGUCUUGUCUUAG | |
| AD-2011253 | usasucu(Gda)AfgCfUfAfgaguuaccuuL96 | | asAfsgguAfaCfUfcuagCfuCfagauasasu | | AUUAUCUGAGCUAGAGUUACCUA | |
| AD-2011254 | asascug(Uda)AfaUfAfGfgauauagcuuL96 | | asAfsgcuAfuAfUfccuaUfuAfcaguusgsa | | UCAACUGUAAUAGGAUAUAGCUA | |
| AD-2011255 | asusgaa(Ada)AfgGfGfCfuuuauuuucuL96 | | asGfsaaaAfuAfAfagccCfuUfuucausgsu | | ACAUGAAAAGGGCUUUAUUUUCA | |
| AD-2011256 | csasgaa(Ada)GfuAfUfCfccuagucuuuL96 | | asAfsagaCfuAfGfggauAfcUfuucugsusa | | UACAGAAAGUAUCCCUAGUCUUA | |
| AD-2011257 | usgscca(Gda)CfuUfUfUfuaucuuucuuL96 | | asAfsgaaAfgAfUfaaaaAfgCfuggcasgsc | | GCUGCCAGCUUUUUAUCUUUCUC | |
| AD-2011258 | csusuuu(Gda)AfgGfUfGfaauauaauuuL96 | | asAfsauuAfuAfUfucacCfuCfaaaagsasg | | CUCUUUUGAGGUGAAUAUAAUUU | |
| AD-2011259 | uscsagg(Gda)AfuAfUfCfugaagaacauL96 | | asUfsguuCfuUfCfagauAfuCfccugasasc | | GUUCAGGGAUAUCUGAAGAACAG | |
| AD-2011260 | csasaca(Ada)GfcAfCfGfucaaaagcuuL96 | | asAfsgcuUfuUfGfacguGfcUfuguugsasg | | CUCAACAAGCACGUCAAAAGCUA | |
| AD-2011261 | asusugu(Ada)UfaAfCfAfgaccacuucuL96 | | asGfsaagUfgGfUfcuguUfaUfacaausasu | | AUAUUGUAUAACAGACCACUUCC | |
| AD-2011262 | ususaau(Gda)UfuGfCfCfuuuuauauuuL96 | | asAfsauaUfaAfAfaggcAfaCfauuaasgsc | | GCUUAAUGUUGCCUUUUAUAUUU | |
| AD-2011263 | asusuga(Cda)CfuUfGfGfuuucuuacauL96 | | asUfsguaAfgAfAfaccaAfgGfucaausasu | | AUAUUGACCUUGGUUUCUUACAC | |
| AD-2011264 | usasuga(Uda)CfaAfCfAfgaugagaacuL96 | | asGfsuucUfcAfUfcuguUfgAfucauasusg | | CAUAUGAUCAACAGAUGAGAACU | |
| AD-2011265 | gscsaga(Gda)AfuUfUfCfuuaagugacuL96 | | asGfsucaCfuUfAfagaaAfuCfucugcscsc | | GGGCAGAGAUUUCUUAAGUGACG | |
| AD-2011266 | gsasaga(Gda)UfuUfCfUfuugugaaaauL96 | | asUfsuuuCfaCfAfaagaAfaCfucuucsusa | | UAGAAGAGUUUCUUUGUGAAAAG | |
| AD-2011267 | csasacc(Ada)UfuGfAfAfaugccucaauL96 | | asUfsugaGfgCfAfuuucAfaUfgguugsasg | | CUCAACCAUUGAAAUGCCUCAAC | |
| AD-2011268 | asuscua(Uda)UfuUfGfCfaguccacucuL96 | | asGfsaguGfgAfCfugcaAfaAfuagausgsc | | GCAUCUAUUUUGCAGUCCACUCU | |
| AD-2011269 | gsusuac(Cda)AfuAfUfGfuauucaucuuL96 | | asAfsgauGfaAfUfacauAfuGfguaacsasa | | UUGUUACCAUAUGUAUUCAUCUG | |
| AD-2011270 | ususcuu(Ada)UfcUfUfAfauuuacagguL96 | | asCfscugUfaAfAfuuaaGfaUfaagaascsu | | AGUUCUUAUCUUAAUUUACAGGG | |
| AD-2011271 | ascsagu(Uda)AfuCfUfCfauauuuggcuL96 | | asGfsccaAfaUfAfugagAfuAfacuguscsu | | AGACAGUUAUCUCAUAUUUGGCU | |
| AD-2011272 | asascaa(Uda)UfuUfAfAfuuucaguuguL96 | | asCfsaacUfgAfAfauuaAfaAfuuguusgsg | | CCAACAAUUUUAAUUUCAGUUGA | |
| AD-2011273 | gsgscaa(Gda)GfaAfAfAfuaaaagauuuL96 | | asAfsaucUfuUfUfauuuUfcCfuugccsusg | | CAGGCAAGGAAAAUAAAAGAUUU | |
| AD-2011274 | uscsauc(Gda)UfgAfUfGfcuucucugauL96 | | asUfscagAfgAfAfgcauCfaCfgaugasusa | | UAUCAUCGUGAUGCUUCUCUGAA | |
| AD-2011275 | asasgau(Gda)AfaGfAfGfuuuaguuuuuL96 | | asAfsaaaCfuAfAfacucUfuCfaucuuscsa | | UGAAGAUGAAGAGUUUAGUUUUA | |
| AD-2011276 | csascug(Uda)AfgUfGfAfauuaucugauL96 | | asUfscagAfuAfAfuucaCfuAfcagugscsc | | GGCACUGUAGUGAAUUAUCUGAG | |
| AD-2011277 | csusaca(Ada)CfcUfCfUfagaucugcauL96 | | asUfsgcaGfaUfCfuagaGfgUfuguagscsa | | UGCUACAACCUCUAGAUCUGCAG | |
| AD-2011278 | asusgag(Ada)AfuCfAfAfguauggaaauL96 | | asUfsuucCfaUfAfcuugAfuUfcucauscsa | | UGAUGAGAAUCAAGUAUGGAAAA | |
| AD-2011279 | ususguu(Cda)AfaGfGfGfucaacuguauL96 | | asUfsacaGfuUfGfacccUfuGfaacaascsa | | UGUUGUUCAAGGGUCAACUGUAA | |
| AD-2011280 | gsasacc(Cda)AfuGfAfGfagauacuaguL96 | | asCfsuagUfaUfCfucucAfuGfgguucsasg | | CUGAACCCAUGAGAGAUACUAGA | |
| AD-2011281 | asusgug(Ada)CfaGfUfGfagauuagucuL96 | | asGfsacuAfaUfCfucacUfgUfcacausasu | | AUAUGUGACAGUGAGAUUAGUCA | |
| AD-2011282 | csusauu(Cda)AfuUfAfAfauggaaguguL96 | | asCfsacuUfcCfAfuuuaAfuGfaauagsusa | | UACUAUUCAUUAAAUGGAAGUGG | |
| AD-2011283 | uscsuuu(Uda)AfaAfGfUfugaugagaauL96 | | asUfsucuCfaUfCfaacuUfuAfaaagasusg | | CAUCUUUUAAAGUUGAUGAGAAU | |
| AD-2011284 | gsgsuga(Cda)AfgAfGfUfcagaaaacuuL96 | | asAfsguuUfuCfUfgacuCfuGfucaccscsa | | UGGGUGACAGAGUCAGAAAACUC | |
| AD-2011285 | asasaca(Ada)CfaGfGfUfgauacacucuL96 | | asGfsaguGfuAfUfcaccUfgUfuguuusasu | | AUAAACAACAGGUGAUACACUCA | |
| AD-2011286 | cscsacu(Uda)CfcUfGfAfguagaagaguL96 | | asCfsucuUfcUfAfcucaGfgAfaguggsusc | | GACCACUUCCUGAGUAGAAGAGU | |
| AD-2011287 | asasauc(Uda)UfaUfUfCfugugaggauuL96 | | asAfsuccUfcAfCfagaaUfaAfgauuususa | | UAAAAUCUUAUUCUGUGAGGAUU | |
| AD-2011288 | asgsugg(Ada)AfaGfUfGfuuugguucauL96 | | asUfsgaaCfcAfAfacacUfuUfccacuscsc | | GGAGUGGAAAGUGUUUGGUUCAG | |
| AD-2011289 | ususgua(Uda)UfuUfUfUfcuaugccacuL96 | | asGfsuggCfaUfAfgaaaAfaAfuacaasusu | | AAUUGUAUUUUUUCUAUGCCACA | |
| AD-2011290 | gsusguu(Ada)UfaUfGfUfauuauacacuL96 | | asGfsuguAfuAfAfuacaUfaUfaacacsgsc | | GCGUGUUAUAUGUAUUAUACACU | |
| AD-2011291 | asasaga(Gda)UfaGfAfGfgauguguaauL96 | | asUfsuacAfcAfUfccucUfaCfucuuususa | | UAAAAGAGUAGAGGAUGUGUAAU | |
| AD-2011292 | asgsuuu(Uda)AfaAfAfCfugcacugccuL96 | | asGfsgcaGfuGfCfaguuUfuAfaaacusasa | | UUAGUUUUAAAACUGCACUGCCA | |
| AD-2011293 | asasgau(Uda)UfcCfAfGfugacagaaauL96 | | asUfsuucUfgUfCfacugGfaAfaucuususu | | AAAAGAUUUCCAGUGACAGAAAA | |
| AD-2011294 | ascsugg(Uda)GfgUfUfAfauaugugacuL96 | | asGfsucaCfaUfAfuuaaCfcAfccagususc | | GAACUGGUGGUUAAUAUGUGACA | |
| AD-2011295 | ususgca(Ada)UfuCfAfAfgcccuuguuuL96 | | asAfsacaAfgGfGfcuugAfaUfugcaasgsg | | CCUUGCAAUUCAAGCCCUUGUUG | |
| AD-2011296 | asasgug(Ada)CfgCfCfUfcaucuacaauL96 | | asUfsuguAfgAfUfgaggCfgUfcacuusasa | | UUAAGUGACGCCUCAUCUACAAG | |
| AD-2011297 | asusuuu(Gda)CfaGfGfUfugucuuccauL96 | | asUfsggaAfgAfCfaaccUfgCfaaaauscsa | | UGAUUUUGCAGGUUGUCUUCCAU | |
| AD-2011298 | asusgua(Ada)CfaUfCfAfauauugaccuL96 | | asGfsgucAfaUfAfuugaUfgUfuacausasu | | AUAUGUAACAUCAAUAUUGACCU | |
| AD-2011299 | asasaga(Ada)UfaUfUfCfauguauaaguL96 | | asCfsuuaUfaCfAfugaaUfaUfucuuuscsc | | GGAAAGAAUAUUCAUGUAUAAGU | |
| AD-2011300 | asasgua(Ada)UfaAfCfAfcaaaugaaguL96 | | asCfsuucAfuUfUfguguUfaUfuacuususg | | CAAAGUAAUAACACAAAUGAAGU | |
| AD-2011301 | usascag(Cda)UfgAfCfCfcuugaacauuL96 | | asAfsuguUfcAfAfggguCfaGfcuguasgsa | | UCUACAGCUGACCCUUGAACAUG | |
| AD-2011302 | asusgga(Ada)AfaGfUfAfaggccauacuL96 | | asGfsuauGfgCfCfuuacUfuUfuccausasc | | GUAUGGAAAAGUAAGGCCAUACU | |

TABLE 39
Unmodified Sense and Antisense Strand Sequences of PLN dsRNA
Agents Comprising a GalNAc Conjugate Targeting Ligand

| | | SEQ | Range in | | | SEQ | Range in | |
|---|---|---|---|---|---|---|---|---|
| Duplex | Sense Strand | ID | NM_002667.5 | | Antisense Strand | ID | NM_002667.5 | |
| Name | Sequence 5′ to 3′ | NO. | Start | End | Sequence 5′ to 3′ | NO. | Start | End |
| AD-2004072 | GGUGACAGAGUCAGAAAACUU | | 9 | 29 | AAGUUUUCUGACUCUGUCACCCA | | 7 | 29 |
| AD-2004080 | AAACACCCGUAAGACUUCAUU | | 37 | 57 | AAUGAAGUCUUACGGGUGUUUAG | | 35 | 57 |
| AD-2004095 | UUCAUACAACACAAUACUCUU | | 52 | 72 | AAGAGUAUUGUGUUGUAUGAAGU | | 50 | 72 |
| AD-2004113 | CUAUACUGUGAUGAUCACAGU | | 70 | 90 | ACUGUGAUCAUCACAGUAUAGAG | | 68 | 90 |
| AD-2004142 | CUACCUAAAAGAAGACAGUUU | | 99 | 119 | AAACUGUCUUCUUUUAGGUAGCC | | 97 | 119 |
| AD-2004156 | ACAGUUAUCUCAUAUUUGGCU | | 113 | 133 | AGCCAAAUAUGAGAUAACUGUCU | | 111 | 133 |
| AD-2004176 | UGCCAGCUUUUUAUCUUUCUU | | 133 | 153 | AAGAAAGAUAAAAAGCUGGCAGC | | 131 | 153 |
| AD-2004189 | UCUUUCUCUCGACCACUUAAU | | 146 | 166 | AUUAAGUGGUCGAGAGAAAGAUA | | 144 | 166 |
| AD-2004202 | CACUUAAAACUUCAGACUUCU | | 159 | 179 | AGAAGUCUGAAGUUUUAAGUGGU | | 157 | 179 |
| AD-2004238 | AUGGAGAAAGUCCAAUACCUU | | 195 | 215 | AAGGUAUUGGACUUUCUCCAUGA | | 193 | 215 |
| AD-2004260 | CUCGCUCAGCUAUAAGAAGAU | | 217 | 237 | AUCUUCUUAUAGCUGAGCGAGUG | | 215 | 237 |
| AD-2004284 | CAACCAUUGAAAUGCCUCAAU | | 241 | 261 | AUUGAGGCAUUUCAAUGGUUGAG | | 239 | 261 |
| AD-2004301 | CAACAAGCACGUCAAAAGCUU | | 258 | 278 | AAGCUUUUGACGUGCUUGUUGAG | | 256 | 278 |
| AD-2004319 | CUACAGAAUCUAUUUAUCAAU | | 276 | 296 | AUUGAUAAAUAGAUUCUGUAGCU | | 274 | 296 |
| AD-2004329 | UAUCAAUUUCUGUCUCAUCUU | | 290 | 310 | AAGAUGAGACAGAAAUUGAUAAA | | 288 | 310 |
| AD-2004345 | AUCUUAAUAUGUCUCUUGCUU | | 306 | 326 | AAGCAAGAGACAUAUUAAGAUGA | | 304 | 326 |
| AD-2004360 | UUGCUGAUCUGUAUCAUCGUU | | 321 | 341 | AACGAUGAUACAGAUCAGCAAGA | | 319 | 341 |
| AD-2004373 | UCAUCGUGAUGCUUCUCUGAU | | 334 | 354 | AUCAGAGAAGCAUCACGAUGAUA | | 332 | 354 |
| AD-2004386 | UCUCUGAAGUUCUGCUACAAU | | 347 | 367 | AUUGUAGCAGAACUUCAGAGAAG | | 345 | 367 |
| AD-2004400 | CUACAACCUCUAGAUCUGCAU | | 361 | 381 | AUGCAGAUCUAGAGGUUGUAGCA | | 359 | 381 |
| AD-2004428 | CAUCAGCUUAAAAUCUGUCAU | | 389 | 409 | AUGACAGAUUUUAAGCUGAUGUG | | 387 | 409 |
| AD-2004458 | ACAGGAAAACAAUAUUGUAUU | | 419 | 439 | AAUACAAUAUUGUUUUCCUGUCU | | 417 | 439 |
| AD-2004468 | AUUGUAUAACAGACCACUUCU | | 432 | 452 | AGAAGUGGUCUGUUAUACAAUAU | | 430 | 452 |
| AD-2004481 | CCACUUCCUGAGUAGAAGAGU | | 445 | 465 | ACUCUUCUACUCAGGAAGUGGUC | | 443 | 465 |
| AD-2004495 | GAAGAGUUUCUUUGUGAAAAU | | 459 | 479 | AUUUUCACAAAGAAACUCUUCUA | | 457 | 479 |
| AD-2004518 | CAAGAUUAAGACUAAAACUUU | | 482 | 502 | AAAGUUUUAGUCUUAAUCUUGAC | | 480 | 502 |
| AD-2004539 | GUUACCAUAUGUAUUCAUCUU | | 505 | 525 | AAGAUGAAUACAUAUGGUAACAA | | 503 | 525 |
| AD-2004555 | AUCUGUUGGAUCUUGUAAACU | | 521 | 541 | AGUUUACAAGAUCCAACAGAUGA | | 519 | 541 |
| AD-2004575 | AUGAAAAGGGCUUUAUUUUCU | | 541 | 561 | AGAAAAUAAAGCCCUUUUCAUGU | | 539 | 561 |
| AD-2004594 | GUGUAUAAAAUGCAACUGUUU | | 581 | 601 | AAACAGUUGCAUUUUAUACACUU | | 579 | 601 |
| AD-2004608 | ACUGUUGAUUUCCUCAACAUU | | 595 | 615 | AAUGUUGAGGAAAUCAACAGUUG | | 593 | 615 |
| AD-2004632 | CACAAAUUUCUAUCCCAAAUU | | 619 | 639 | AAUUUGGGAUAGAAAUUUGUGAG | | 617 | 639 |
| AD-2004646 | CCAAAUCUUUUCUGAAGAUGU | | 633 | 653 | ACAUCUUCAGAAAAGAUUUGGGA | | 631 | 653 |
| AD-2004660 | AAGAUGAAGAGUUUAGUUUUU | | 647 | 667 | AAAAACUAAACUCUUCAUCUUCA | | 645 | 667 |
| AD-2004673 | AGUUUUAAAACUGCACUGCCU | | 661 | 681 | AGGCAGUGCAGUUUUAAAACUAA | | 659 | 681 |
| AD-2004689 | UGCCAACAAGUUCACUUCAUU | | 677 | 697 | AAUGAAGUGAACUUGUUGGCAGU | | 675 | 697 |
| AD-2004718 | CUUUUGAGGUGAAUAUAAUUU | | 718 | 738 | AAAUUAUAUUCACCUCAAAAGAG | | 716 | 738 |
| AD-2004747 | AAGUAUUUUUCAGGUCUUCAU | | 769 | 789 | AUGAAGACCUGAAAAAUACUUAG | | 767 | 789 |
| AD-2004763 | UUCACCAAGUAUCAAAGUAAU | | 785 | 805 | AUUACUUUGAUACUUGGUGAAGA | | 783 | 805 |
| AD-2004777 | AAGUAAUAACACAAAUGAAGU | | 799 | 819 | ACUUCAUUUGUGUUAUUACUUUG | | 797 | 819 |
| AD-2004790 | AAUGAAGUGUCAUUAUUCAAU | | 812 | 832 | AUUGAAUAAUGACACUUCAUUUG | | 810 | 832 |
| AD-2004806 | UCAAAAUAGUCCACUGACUCU | | 828 | 848 | AGAGUCAGUGGACUAUUUUGAAU | | 826 | 848 |
| AD-2004825 | CCUCACAUCUGUUAUCUUAUU | | 847 | 867 | AAUAAGAUAACAGAUGUGAGGAG | | 845 | 867 |
| AD-2004837 | AACUAUUUGUAGUAACUAUCU | | 874 | 894 | AGAUAGUUACUACAAAUAGUUCU | | 872 | 894 |
| AD-2004853 | UAUCAGAAUCUACAUUCUAAU | | 890 | 910 | AUUAGAAUGUAGAUUCUGAUAGU | | 888 | 910 |
| AD-2004872 | UUGUAUUUUUUCUAUGCCACU | | 918 | 938 | AGUGGCAUAGAAAAAAUACAAUU | | 916 | 938 |
| AD-2004885 | AUGCCACAUUAACAUCUUUUU | | 931 | 951 | AAAAAGAUGUUAAUGUGGCAUAG | | 929 | 951 |
| AD-2004896 | UCUUUUAAAGUUGAUGAGAAU | | 945 | 965 | AUUCUCAUCAACUUUAAAAGAUG | | 943 | 965 |
| AD-2004909 | AUGAGAAUCAAGUAUGGAAAU | | 958 | 978 | AUUUCCAUACUUGAUUCUCAUCA | | 956 | 978 |
| AD-2004922 | AUGGAAAAGUAAGGCCAUACU | | 971 | 991 | AGUAUGGCCUUACUUUUCCAUAC | | 969 | 991 |
| AD-2004935 | GCCAUACUCUUACAUAAUAAU | | 984 | 1004 | AUUAUUAUGUAAGAGUAUGGCCU | | 982 | 1004 |
| AD-2004950 | GAAUCACAGAAUUCUAGUACU | | 1030 | 1050 | AGUACUAGAAUUCUGUGAUUCUU | | 1028 | 1050 |
| AD-2004970 | AUGUAGGUAAAUCAUAAAUCU | | 1050 | 1070 | AGAUUUAUGAUUUACCUACAUGU | | 1048 | 1070 |
| AD-2004989 | CUGUUCUAAGACAUAUGAUCU | | 1069 | 1089 | AGAUCAUAUGUCUUAGAACAGAU | | 1067 | 1089 |
| AD-2005002 | UAUGAUCAACAGAUGAGAACU | | 1082 | 1102 | AGUUCUCAUCUGUUGAUCAUAUG | | 1080 | 1102 |
| AD-2005020 | ACUGGUGGUUAAUAUGUGACU | | 1100 | 1120 | AGUCACAUAUUAACCACCAGUUC | | 1098 | 1120 |
| AD-2005033 | AUGUGACAGUGAGAUUAGUCU | | 1113 | 1133 | AGACUAAUCUCACUGUCACAUAU | | 1111 | 1133 |
| AD-2005051 | CAUAUCACUAAUAUACUAACU | | 1132 | 1152 | AGUUAGUAUAUUAGUGAUAUGAC | | 1130 | 1152 |
| AD-2005067 | UAACAACAGAAUCUAAUCUUU | | 1148 | 1168 | AAAGAUUAGAUUCUGUUGUUAGU | | 1146 | 1168 |
| AD-2005082 | UCUUCAUUUAAGGCACUGUAU | | 1164 | 1184 | AUACAGUGCCUUAAAUGAAGAUU | | 1162 | 1184 |
| AD-2005095 | CACUGUAGUGAAUUAUCUGAU | | 1177 | 1197 | AUCAGAUAAUUCACUACAGUGCC | | 1175 | 1197 |
| AD-2005108 | UAUCUGAGCUAGAGUUACCUU | | 1190 | 1210 | AAGGUAACUCUAGCUCAGAUAAU | | 1188 | 1210 |
| AD-2005129 | GCUUACCAUACUAUAUCUUUU | | 1211 | 1231 | AAAAGAUAUAGUAUGGUAAGCUA | | 1209 | 1231 |
| AD-2005146 | UUUGGAAUCAUGAAACCUUAU | | 1228 | 1248 | AUAAGGUUUCAUGAUUCCAAAGA | | 1226 | 1248 |
| AD-2005164 | UAAGACUUCAGAAUGAUUUUU | | 1246 | 1266 | AAAAAUCAUUCUGAAGUCUUAAG | | 1244 | 1266 |
| AD-2005179 | AUUUUGCAGGUUGUCUUCCAU | | 1261 | 1281 | AUGGAAGACAACCUGCAAAAUCA | | 1259 | 1281 |
| AD-2005193 | CUUCCAUUCCAGCCUAACAUU | | 1275 | 1295 | AAUGUUAGGCUGGAAUGGAAGAC | | 1273 | 1295 |
| AD-2005221 | GGCAAGGAAAAUAAAAGAUUU | | 1303 | 1323 | AAAUCUUUUAUUUUCCUUGCCUG | | 1301 | 1323 |
| AD-2005234 | AAGAUUUCCAGUGACAGAAAU | | 1317 | 1337 | AUUUCUGUCACUGGAAAUCUUUU | | 1315 | 1337 |
| AD-2005250 | UAUGAAUUCUCUCUCCAAAUU | | 1368 | 1388 | AAUUUGGAGAGAGAAUUCAUAUA | | 1366 | 1388 |
| AD-2005274 | GAACUUGUUGGCCCAUCUAUU | | 1419 | 1439 | AAUAGAUGGGCCAACAAGUUCAU | | 1417 | 1439 |
| AD-2005287 | CAUCUAUUACAUCUACAGCUU | | 1432 | 1452 | AAGCUGUAGAUGUAAUAGAUGGG | | 1430 | 1452 |
| AD-2005300 | UACAGCUGACCCUUGAACAUU | | 1445 | 1465 | AAUGUUCAAGGGUCAGCUGUAGA | | 1443 | 1465 |
| AD-2005325 | CAAAAUCUUAACUACCUAAUU | | 1498 | 1518 | AAUUAGGUAGUUAAGAUUUUGCG | | 1496 | 1518 |
| AD-2005350 | ACUAUUGACCAUAAACCUUAU | | 1523 | 1543 | AUAAGGUUUAUGGUCAAUAGUAG | | 1521 | 1543 |
| AD-2005365 | CCUUACUGAUAACAUAAACAU | | 1538 | 1558 | AUGUUUAUGUUAUCAGUAAGGUU | | 1536 | 1558 |
| AD-2005384 | ACACAUAUUUUGCGUGUUAUU | | 1566 | 1586 | AAUAACACGCAAAAUAUGUGUUA | | 1564 | 1586 |
| AD-2005396 | GUGUUAUAUGUAUUAUACACU | | 1579 | 1599 | AGUGUAUAAUACAUAUAACACGC | | 1577 | 1599 |
| AD-2005409 | CUAUAUUCCUACAAUAAAGUU | | 1598 | 1618 | AACUUUAUUGUAGGAAUAUAGUG | | 1596 | 1618 |
| AD-2005429 | AAGCUAGAGAAAAUGUUAUUU | | 1618 | 1638 | AAAUAACAUUUUCUCUAGCUUAC | | 1616 | 1638 |
| AD-2005447 | CUAUUCAUUAAAUGGAAGUGU | | 1670 | 1690 | ACACUUCCAUUUAAUGAAUAGUA | | 1668 | 1690 |
| AD-2005471 | AACAUAAAAGUCUUCAUUCUU | | 1694 | 1714 | AAGAAUGAAGACUUUUAUGUUGA | | 1692 | 1714 |
| AD-2005485 | CAUUCUCAUUGUCUUCACAUU | | 1708 | 1728 | AAUGUGAAGACAAUGAGAAUGAA | | 1706 | 1728 |
| AD-2005527 | GGUCUUGCAGUCUUGUCUUAU | | 1773 | 1793 | AUAAGACAAGACUGCAAGACCAA | | 1771 | 1793 |
| AD-2005530 | AAAGAAUAUUCAUGUAUAAGU | | 1812 | 1832 | ACUUAUACAUGAAUAUUCUUUCC | | 1810 | 1832 |
| AD-2005557 | UUGCAAUUCAAGCCCUUGUUU | | 1839 | 1859 | AAACAAGGGCUUGAAUUGCAAGG | | 1837 | 1859 |
| AD-2005575 | UUGUUCAAGGGUCAACUGUAU | | 1857 | 1877 | AUACAGUUGACCCUUGAACAACA | | 1855 | 1877 |
| AD-2005588 | AACUGUAAUAGGAUAUAGCUU | | 1870 | 1890 | AAGCUAUAUCCUAUUACAGUUGA | | 1868 | 1890 |
| AD-2005604 | CUAUUUUUCUUCCUCUAUCAU | | 1888 | 1908 | AUGAUAGAGGAAGAAAAAUAGCU | | 1886 | 1908 |
| AD-2005617 | UCUAUCAACCAAAUGGUAAGU | | 1901 | 1921 | ACUUACCAUUUGGUUGAUAGAGG | | 1899 | 1921 |
| AD-2005638 | AUCUAUUUUGCAGUCCACUCU | | 1922 | 1942 | AGAGUGGACUGCAAAAUAGAUGC | | 1920 | 1942 |
| AD-2005658 | UACUGAGCUAAAUUAUAGAUU | | 1942 | 1962 | AAUCUAUAAUUUAGCUCAGUAGA | | 1940 | 1962 |
| AD-2005678 | CCAGCUAUGCUAUUUAUAAUU | | 1962 | 1982 | AAUUAUAAAUAGCAUAGCUGGAU | | 1960 | 1982 |
| AD-2005687 | UUUUCAAUUUCUCCUCUGACU | | 2001 | 2021 | AGUCAGAGGAGAAAUUGAAAAUU | | 1999 | 2021 |
| AD-2005704 | GACCAUUUCAGAACAUCUUCU | | 2018 | 2038 | AGAAGAUGUUCUGAAAUGGUCAG | | 2016 | 2038 |
| AD-2005720 | CUUCCAAUAACUCAUAAAACU | | 2034 | 2054 | AGUUUUAUGAGUUAUUGGAAGAU | | 2032 | 2054 |
| AD-2005737 | CAACUGAAGUAAAAUUGAGUU | | 2053 | 2073 | AACUCAAUUUUACUUCAGUUGUU | | 2051 | 2073 |
| AD-2005760 | GGAAAAUAUAUUCACCAAACU | | 2076 | 2096 | AGUUUGGUGAAUAUAUUUUCCAG | | 2074 | 2096 |
| AD-2005826 | AAACUUUGGUAAUUUAAGUUU | | 2092 | 2112 | AAACUUAAAUUACCAAAGUUUGG | | 2090 | 2112 |
| AD-2005849 | AAAAUAGUUUACACCUAUACU | | 2135 | 2155 | AGUAUAGGUGUAAACUAUUUUAG | | 2133 | 2155 |
| AD-2005862 | CCUAUACUGCAUAAUCCAACU | | 2148 | 2168 | AGUUGGAUUAUGCAGUAUAGGUG | | 2146 | 2168 |
| AD-2005923 | AACAAUUUUAAUUUCAGUUGU | | 2165 | 2185 | ACAACUGAAAUUAAAAUUGUUGG | | 2163 | 2185 |
| AD-2005942 | AGACAUGUUACUAAUAUAACU | | 2186 | 2206 | AGUUAUAUUAGUAACAUGUCUUC | | 2184 | 2206 |
| AD-2005954 | AAAGAGUAGAGGAUGUGUAAU | | 2214 | 2234 | AUUACACAUCCUCUACUCUUUUA | | 2212 | 2234 |
| AD-2006018 | GUGUAAUUAACCAUAUCUUCU | | 2228 | 2248 | AGAAGAUAUGGUUAAUUACACAU | | 2226 | 2248 |
| AD-2006035 | UUCUAAAACAUGGUUACUAAU | | 2245 | 2265 | AUUAGUAACCAUGUUUUAGAAGA | | 2243 | 2265 |
| AD-2006046 | UUACUAAAAGAAUAUGUAACU | | 2258 | 2278 | AGUUACAUAUUCUUUUAGUAACC | | 2256 | 2278 |
| AD-2006056 | AUGUAACAUCAAUAUUGACCU | | 2271 | 2291 | AGGUCAAUAUUGAUGUUACAUAU | | 2269 | 2291 |
| AD-2006119 | AUUGACCUUGGUUUCUUACAU | | 2284 | 2304 | AUGUAAGAAACCAAGGUCAAUAU | | 2282 | 2304 |
| AD-2006132 | UCUUACACAAGUGUUGCUAAU | | 2297 | 2317 | AUUAGCAACACUUGUGUAAGAAA | | 2295 | 2317 |
| AD-2006150 | AACUCAAUAGUGAAGGAGACU | | 2315 | 2335 | AGUCUCCUUCACUAUUGAGUUAG | | 2313 | 2335 |
| AD-2006164 | GGAGACACUAUUAAAUUUUCU | | 2329 | 2349 | AGAAAAUUUAAUAGUGUCUCCUU | | 2327 | 2349 |
| AD-2006234 | GAACCCAUGAGAGAUACUAGU | | 2350 | 2370 | ACUAGUAUCUCUCAUGGGUUCAG | | 2348 | 2370 |
| AD-2006242 | AGUGGAAAGUGUUUGGUUCAU | | 2378 | 2398 | AUGAACCAAACACUUUCCACUCC | | 2376 | 2398 |
| AD-2006259 | UCAGGGAUAUCUGAAGAACAU | | 2395 | 2415 | AUGUUCUUCAGAUAUCCCUGAAC | | 2393 | 2415 |
| AD-2006334 | GCAGAGAUUUCUUAAGUGACU | | 2420 | 2440 | AGUCACUUAAGAAAUCUCUGCCC | | 2418 | 2440 |
| AD-2006347 | AAGUGACGCCUCAUCUACAAU | | 2433 | 2453 | AUUGUAGAUGAGGCGUCACUUAA | | 2431 | 2453 |
| AD-2006362 | UACAAGCUGGAAAUUCCUAAU | | 2448 | 2468 | AUUAGGAAUUUCCAGCUUGUAGA | | 2446 | 2468 |
| AD-2006437 | AGUAGAAAGCUUAUAAACAAU | | 2473 | 2493 | AUUGUUUAUAAGCUUUCUACUUG | | 2471 | 2493 |
| AD-2006451 | AAACAACAGGUGAUACACUCU | | 2487 | 2507 | AGAGUGUAUCACCUGUUGUUUAU | | 2485 | 2507 |
| AD-2006525 | CACUGGUUUUAGUAAAUUACU | | 2511 | 2531 | AGUAAUUUACUAAAACCAGUGAG | | 2509 | 2531 |
| AD-2006548 | CAGAAAGUAUCCCUAGUCUUU | | 2536 | 2556 | AAAGACUAGGGAUACUUUCUGUA | | 2534 | 2556 |
| AD-2006626 | GUGGAAAAUUUGAACUGAUUU | | 2564 | 2584 | AAAUCAGUUCAAAUUUUCCACUU | | 2562 | 2584 |
| AD-2006640 | CUGAUUAGUCAUAUUCCUUUU | | 2578 | 2598 | AAAAGGAAUAUGACUAAUCAGUU | | 2576 | 2598 |
| AD-2006658 | UUGAUUACACUGUUUGUUACU | | 2596 | 2616 | AGUAACAAACAGUGUAAUCAAAG | | 2594 | 2616 |
| AD-2006731 | UCUCAGUAAACAGAAAUAACU | | 2624 | 2644 | AGUUAUUUCUGUUUACUGAGAAA | | 2622 | 2644 |
| AD-2006743 | UCUUCAUUCUUUGAUAGAAAU | | 2656 | 2676 | AUUUCUAUCAAAGAAUGAAGAAC | | 2654 | 2676 |
| AD-2006751 | AAAUCUUAUUCUGUGAGGAUU | | 2679 | 2699 | AAUCCUCACAGAAUAAGAUUUUA | | 2677 | 2699 |
| AD-2006764 | UGAGGAUUACAGAAUACUAUU | | 2692 | 2712 | AAUAGUAUUCUGUAAUCCUCACA | | 2690 | 2712 |
| AD-2006839 | CUUCAUCAUAAAGUGUAAAGU | | 2754 | 2774 | ACUUUACACUUUAUGAUGAAGAA | | 2752 | 2774 |
| AD-2006862 | AUGCUCAAAUAUGUUCUACUU | | 2820 | 2840 | AAGUAGAACAUAUUUGAGCAUUU | | 2818 | 2840 |
| AD-2006926 | UCUACUAUAGAAUAAGUUCUU | | 2834 | 2854 | AAGAACUUAUUCUAUAGUAGAAC | | 2832 | 2854 |
| AD-2006935 | UUCUUAUCUUAAUUUACAGGU | | 2850 | 2870 | ACCUGUAAAUUAAGAUAAGAACU | | 2848 | 2870 |
| AD-2006965 | UUAAUGUUGCCUUUUAUAUUU | | 2894 | 2914 | AAAUAUAAAAGGCAACAUUAAGC | | 2892 | 2914 |
| AD-2007034 | AAAUCAGAAUCACUAUAUUAU | | 2947 | 2967 | AUAAUAUAGUGAUUCUGAUUUGC | | 2945 | 2967 |

TABLE 40
Modified Sense and Antisense Strand Sequences of PLN dsRNA Agents Comprising a GalNAc Conjugate Targeting Ligand

| Duplex Name | Sense Strand Sequence 5′ to 3′ | SEQ ID NO. | Antisense Strand Sequence 5′ to 3′ | SEQ ID NO. | mRNA target sequence | SEQ ID NO. |
|---|---|---|---|---|---|---|
| AD-2004072 | gsgsugacAfgAfGfUfcagaaaacuuL96 | | asAfsguuUfuCfUfgacuCfuGfucaccscsa | | UGGGUGACAGAGUCAGAAAACUC | |
| AD-2004080 | asasacacCfcGfUfAfagacuucauuL96 | | asAfsugaAfgUfCfuuacGfgGfuguuusasg | | CUAAACACCCGUAAGACUUCAUA | |
| AD-2004095 | ususcauaCfaAfCfAfcaauacucuuL96 | | asAfsgagUfaUfUfguguUfgUfaugaasgsu | | ACUUCAUACAACACAAUACUCUA | |
| AD-2004113 | csusauacUfgUfGfAfugaucacaguL96 | | asCfsuguGfaUfCfaucaCfaGfuauagsasg | | CUCUAUACUGUGAUGAUCACAGC | |
| AD-2004142 | csusaccuAfaAfAfGfaagacaguuuL96 | | asAfsacuGfuCfUfucuuUfuAfgguagscsc | | GGCUACCUAAAAGAAGACAGUUA | |
| AD-2004156 | ascsaguuAfuCfUfCfauauuuggcuL96 | | asGfsccaAfaUfAfugagAfuAfacuguscsu | | AGACAGUUAUCUCAUAUUUGGCU | |
| AD-2004176 | usgsccagCfuUfUfUfuaucuuucuuL96 | | asAfsgaaAfgAfUfaaaaAfgCfuggcasgsc | | GCUGCCAGCUUUUUAUCUUUCUC | |
| AD-2004189 | uscsuuucUfcUfCfGfaccacuuaauL96 | | asUfsuaaGfuGfGfucgaGfaGfaaagasusa | | UAUCUUUCUCUCGACCACUUAAA | |
| AD-2004202 | csascuuaAfaAfCfUfucagacuucuL96 | | asGfsaagUfcUfGfaaguUfuUfaagugsgsu | | ACCACUUAAAACUUCAGACUUCC | |
| AD-2004238 | asusggagAfaAfGfUfccaauaccuuL96 | | asAfsgguAfuUfGfgacuUfuCfuccausgsa | | UCAUGGAGAAAGUCCAAUACCUC | |
| AD-2004260 | csuscgcuCfaGfCfUfauaagaagauL96 | | asUfscuuCfuUfAfuagcUfgAfgcgagsusg | | CACUCGCUCAGCUAUAAGAAGAG | |
| AD-2004284 | csasaccaUfuGfAfAfaugccucaauL96 | | asUfsugaGfgCfAfuuucAfaUfgguugsasg | | CUCAACCAUUGAAAUGCCUCAAC | |
| AD-2004301 | csasacaaGfcAfCfGfucaaaagcuuL96 | | asAfsgcuUfuUfGfacguGfcUfuguugsasg | | CUCAACAAGCACGUCAAAAGCUA | |
| AD-2004319 | csusacagAfaUfCfUfauuuaucaauL96 | | asUfsugaUfaAfAfuagaUfuCfuguagscsu | | AGCUACAGAAUCUAUUUAUCAAU | |
| AD-2004329 | usasucaaUfuUfCfUfgucucaucuuL96 | | asAfsgauGfaGfAfcagaAfaUfugauasasa | | UUUAUCAAUUUCUGUCUCAUCUU | |
| AD-2004345 | asuscuuaAfuAfUfGfucucuugcuuL96 | | asAfsgcaAfgAfGfacauAfuUfaagausgsa | | UCAUCUUAAUAUGUCUCUUGCUG | |
| AD-2004360 | ususgcugAfuCfUfGfuaucaucguuL96 | | asAfscgaUfgAfUfacagAfuCfagcaasgsa | | UCUUGCUGAUCUGUAUCAUCGUG | |
| AD-2004373 | uscsaucgUfgAfUfGfcuucucugauL96 | | asUfscagAfgAfAfgcauCfaCfgaugasusa | | UAUCAUCGUGAUGCUUCUCUGAA | |
| AD-2004386 | uscsucugAfaGfUfUfcugcuacaauL96 | | asUfsuguAfgCfAfgaacUfuCfagagasasg | | CUUCUCUGAAGUUCUGCUACAAC | |
| AD-2004400 | csusacaaCfcUfCfUfagaucugcauL96 | | asUfsgcaGfaUfCfuagaGfgUfuguagscsa | | UGCUACAACCUCUAGAUCUGCAG | |
| AD-2004428 | csasucagCfuUfAfAfaaucugucauL96 | | asUfsgacAfgAfUfuuuaAfgCfugaugsusg | | CACAUCAGCUUAAAAUCUGUCAU | |
| AD-2004458 | ascsaggaAfaAfCfAfauauuguauuL96 | | asAfsuacAfaUfAfuuguUfuUfccuguscsu | | AGACAGGAAAACAAUAUUGUAUA | |
| AD-2004468 | asusuguaUfaAfCfAfgaccacuucuL96 | | asGfsaagUfgGfUfcuguUfaUfacaausasu | | AUAUUGUAUAACAGACCACUUCC | |
| AD-2004481 | cscsacuuCfcUfGfAfguagaagaguL96 | | asCfsucuUfcUfAfcucaGfgAfaguggsusc | | GACCACUUCCUGAGUAGAAGAGU | |
| AD-2004495 | gsasagagUfuUfCfUfuugugaaaauL96 | | asUfsuuuCfaCfAfaagaAfaCfucuucsusa | | UAGAAGAGUUUCUUUGUGAAAAG | |
| AD-2004518 | csasagauUfaAfGfAfcuaaaacuuuL96 | | asAfsaguUfuUfAfgucuUfaAfucuugsasc | | GUCAAGAUUAAGACUAAAACUUA | |
| AD-2004539 | gsusuaccAfuAfUfGfuauucaucuuL96 | | asAfsgauGfaAfUfacauAfuGfguaacsasa | | UUGUUACCAUAUGUAUUCAUCUG | |
| AD-2004555 | asuscuguUfgGfAfUfcuuguaaacuL96 | | asGfsuuuAfcAfAfgaucCfaAfcagausgsa | | UCAUCUGUUGGAUCUUGUAAACA | |
| AD-2004575 | asusgaaaAfgGfGfCfuuuauuuucuL96 | | asGfsaaaAfuAfAfagccCfuUfuucausgsu | | ACAUGAAAAGGGCUUUAUUUUCA | |
| AD-2004594 | gsusguauAfaAfAfUfgcaacuguuuL96 | | asAfsacaGfuUfGfcauuUfuAfuacacsusu | | AAGUGUAUAAAAUGCAACUGUUG | |
| AD-2004608 | ascsuguuGfaUfUfUfccucaacauuL96 | | asAfsuguUfgAfGfgaaaUfcAfacagususg | | CAACUGUUGAUUUCCUCAACAUG | |
| AD-2004632 | csascaaaUfuUfCfUfaucccaaauuL96 | | asAfsuuuGfgGfAfuagaAfaUfuugugsasg | | CUCACAAAUUUCUAUCCCAAAUC | |
| AD-2004646 | cscsaaauCfuUfUfUfcugaagauguL96 | | asCfsaucUfuCfAfgaaaAfgAfuuuggsgsa | | UCCCAAAUCUUUUCUGAAGAUGA | |
| AD-2004660 | asasgaugAfaGfAfGfuuuaguuuuuL96 | | asAfsaaaCfuAfAfacucUfuCfaucuuscsa | | UGAAGAUGAAGAGUUUAGUUUUA | |
| AD-2004673 | asgsuuuuAfaAfAfCfugcacugccuL96 | | asGfsgcaGfuGfCfaguuUfuAfaaacusasa | | UUAGUUUUAAAACUGCACUGCCA | |
| AD-2004689 | usgsccaaCfaAfGfUfucacuucauuL96 | | asAfsugaAfgUfGfaacuUfgUfuggcasgsu | | ACUGCCAACAAGUUCACUUCAUA | |
| AD-2004718 | csusuuugAfgGfUfGfaauauaauuuL96 | | asAfsauuAfuAfUfucacCfuCfaaaagsasg | | CUCUUUUGAGGUGAAUAUAAUUU | |
| AD-2004747 | asasguauUfuUfUfCfaggucuucauL96 | | asUfsgaaGfaCfCfugaaAfaAfuacuusasg | | CUAAGUAUUUUUCAGGUCUUCAC | |
| AD-2004763 | ususcaccAfaGfUfAfucaaaguaauL96 | | asUfsuacUfuUfGfauacUfuGfgugaasgsa | | UCUUCACCAAGUAUCAAAGUAAU | |
| AD-2004777 | asasguaaUfaAfCfAfcaaaugaaguL96 | | asCfsuucAfuUfUfguguUfaUfuacuususg | | CAAAGUAAUAACACAAAUGAAGU | |
| AD-2004790 | asasugaaGfuGfUfCfauuauucaauL96 | | asUfsugaAfuAfAfugacAfcUfucauususg | | CAAAUGAAGUGUCAUUAUUCAAA | |
| AD-2004806 | uscsaaaaUfaGfUfCfcacugacucuL96 | | asGfsaguCfaGfUfggacUfaUfuuugasasu | | AUUCAAAAUAGUCCACUGACUCC | |
| AD-2004825 | cscsucacAfuCfUfGfuuaucuuauuL96 | | asAfsuaaGfaUfAfacagAfuGfugaggsasg | | CUCCUCACAUCUGUUAUCUUAUU | |
| AD-2004837 | asascuauUfuGfUfAfguaacuaucuL96 | | asGfsauaGfuUfAfcuacAfaAfuaguuscsu | | AGAACUAUUUGUAGUAACUAUCA | |
| AD-2004853 | usasucagAfaUfCfUfacauucuaauL96 | | asUfsuagAfaUfGfuagaUfuCfugauasgsu | | ACUAUCAGAAUCUACAUUCUAAA | |
| AD-2004872 | ususguauUfuUfUfUfcuaugccacuL96 | | asGfsuggCfaUfAfgaaaAfaAfuacaasusu | | AAUUGUAUUUUUUCUAUGCCACA | |
| AD-2004885 | asusgccaCfaUfUfAfacaucuuuuuL96 | | asAfsaaaGfaUfGfuuaaUfgUfggcausasg | | CUAUGCCACAUUAACAUCUUUUA | |
| AD-2004896 | uscsuuuuAfaAfGfUfugaugagaauL96 | | asUfsucuCfaUfCfaacuUfuAfaaagasusg | | CAUCUUUUAAAGUUGAUGAGAAU | |
| AD-2004909 | asusgagaAfuCfAfAfguauggaaauL96 | | asUfsuucCfaUfAfcuugAfuUfcucauscsa | | UGAUGAGAAUCAAGUAUGGAAAA | |
| AD-2004922 | asusggaaAfaGfUfAfaggccauacuL96 | | asGfsuauGfgCfCfuuacUfuUfuccausasc | | GUAUGGAAAAGUAAGGCCAUACU | |
| AD-2004935 | gscscauaCfuCfUfUfacauaauaauL96 | | asUfsuauUfaUfGfuaagAfgUfauggcscsu | | AGGCCAUACUCUUACAUAAUAAA | |
| AD-2004950 | gsasaucaCfaGfAfAfuucuaguacuL96 | | asGfsuacUfaGfAfauucUfgUfgauucsusu | | AAGAAUCACAGAAUUCUAGUACA | |
| AD-2004970 | asusguagGfuAfAfAfucauaaaucuL96 | | asGfsauuUfaUfGfauuuAfcCfuacausgsu | | ACAUGUAGGUAAAUCAUAAAUCU | |
| AD-2004989 | csusguucUfaAfGfAfcauaugaucuL96 | | asGfsaucAfuAfUfgucuUfaGfaacagsasu | | AUCUGUUCUAAGACAUAUGAUCA | |
| AD-2005002 | usasugauCfaAfCfAfgaugagaacuL96 | | asGfsuucUfcAfUfcuguUfgAfucauasusg | | CAUAUGAUCAACAGAUGAGAACU | |
| AD-2005020 | ascsugguGfgUfUfAfauaugugacuL96 | | asGfsucaCfaUfAfuuaaCfcAfccagususc | | GAACUGGUGGUUAAUAUGUGACA | |
| AD-2005033 | asusgugaCfaGfUfGfagauuagucuL96 | | asGfsacuAfaUfCfucacUfgUfcacausasu | | AUAUGUGACAGUGAGAUUAGUCA | |
| AD-2005051 | csasuaucAfcUfAfAfuauacuaacuL96 | | asGfsuuaGfuAfUfauuaGfuGfauaugsasc | | GUCAUAUCACUAAUAUACUAACA | |
| AD-2005067 | usasacaaCfaGfAfAfucuaaucuuuL96 | | asAfsagaUfuAfGfauucUfgUfuguuasgsu | | ACUAACAACAGAAUCUAAUCUUC | |
| AD-2005082 | uscsuucaUfuUfAfAfggcacuguauL96 | | asUfsacaGfuGfCfcuuaAfaUfgaagasusu | | AAUCUUCAUUUAAGGCACUGUAG | |
| AD-2005095 | csascuguAfgUfGfAfauuaucugauL96 | | asUfscagAfuAfAfuucaCfuAfcagugscsc | | GGCACUGUAGUGAAUUAUCUGAG | |
| AD-2005108 | usasucugAfgCfUfAfgaguuaccuuL96 | | asAfsgguAfaCfUfcuagCfuCfagauasasu | | AUUAUCUGAGCUAGAGUUACCUA | |
| AD-2005129 | gscsuuacCfaUfAfCfuauaucuuuuL96 | | asAfsaagAfuAfUfaguaUfgGfuaagcsusa | | UAGCUUACCAUACUAUAUCUUUG | |
| AD-2005146 | ususuggaAfuCfAfUfgaaaccuuauL96 | | asUfsaagGfuUfUfcaugAfuUfccaaasgsa | | UCUUUGGAAUCAUGAAACCUUAA | |
| AD-2005164 | usasagacUfuCfAfGfaaugauuuuuL96 | | asAfsaaaUfcAfUfucugAfaGfucuuasasg | | CUUAAGACUUCAGAAUGAUUUUG | |
| AD-2005179 | asusuuugCfaGfGfUfugucuuccauL96 | | asUfsggaAfgAfCfaaccUfgCfaaaauscsa | | UGAUUUUGCAGGUUGUCUUCCAU | |
| AD-2005193 | csusuccaUfuCfCfAfgccuaacauuL96 | | asAfsuguUfaGfGfcuggAfaUfggaagsasc | | GUCUUCCAUUCCAGCCUAACAUC | |
| AD-2005221 | gsgscaagGfaAfAfAfuaaaagauuuL96 | | asAfsaucUfuUfUfauuuUfcCfuugccsusg | | CAGGCAAGGAAAAUAAAAGAUUU | |
| AD-2005234 | asasgauuUfcCfAfGfugacagaaauL96 | | asUfsuucUfgUfCfacugGfaAfaucuususu | | AAAAGAUUUCCAGUGACAGAAAA | |
| AD-2005250 | usasugaaUfuCfUfCfucuccaaauuL96 | | asAfsuuuGfgAfGfagagAfaUfucauasusa | | UAUAUGAAUUCUCUCUCCAAAUA | |
| AD-2005274 | gsasacuuGfuUfGfGfcccaucuauuL96 | | asAfsuagAfuGfGfgccaAfcAfaguucsasu | | AUGAACUUGUUGGCCCAUCUAUU | |
| AD-2005287 | csasucuaUfuAfCfAfucuacagcuuL96 | | asAfsgcuGfuAfGfauguAfaUfagaugsgsg | | CCCAUCUAUUACAUCUACAGCUG | |
| AD-2005300 | usascagcUfgAfCfCfcuugaacauuL96 | | asAfsuguUfcAfAfggguCfaGfcuguasgsa | | UCUACAGCUGACCCUUGAACAUG | |
| AD-2005325 | csasaaauCfuUfAfAfcuaccuaauuL96 | | asAfsuuaGfgUfAfguuaAfgAfuuuugscsg | | CGCAAAAUCUUAACUACCUAAUA | |
| AD-2005350 | ascsuauuGfaCfCfAfuaaaccuuauL96 | | asUfsaagGfuUfUfauggUfcAfauagusasg | | CUACUAUUGACCAUAAACCUUAC | |
| AD-2005365 | cscsuuacUfgAfUfAfacauaaacauL96 | | asUfsguuUfaUfGfuuauCfaGfuaaggsusu | | AACCUUACUGAUAACAUAAACAG | |
| AD-2005384 | ascsacauAfuUfUfUfgcguguuauuL96 | | asAfsuaaCfaCfGfcaaaAfuAfugugususa | | UAACACAUAUUUUGCGUGUUAUA | |
| AD-2005396 | gsusguuaUfaUfGfUfauuauacacuL96 | | asGfsuguAfuAfAfuacaUfaUfaacacsgsc | | GCGUGUUAUAUGUAUUAUACACU | |
| AD-2005409 | csusauauUfcCfUfAfcaauaaaguuL96 | | asAfscuuUfaUfUfguagGfaAfuauagsusg | | CACUAUAUUCCUACAAUAAAGUA | |
| AD-2005429 | asasgcuaGfaGfAfAfaauguuauuuL96 | | asAfsauaAfcAfUfuuucUfcUfagcuusasc | | GUAAGCUAGAGAAAAUGUUAUUU | |
| AD-2005447 | csusauucAfuUfAfAfauggaaguguL96 | | asCfsacuUfcCfAfuuuaAfuGfaauagsusa | | UACUAUUCAUUAAAUGGAAGUGG | |
| AD-2005471 | asascauaAfaAfGfUfcuucauucuuL96 | | asAfsgaaUfgAfAfgacuUfuUfauguusgsa | | UCAACAUAAAAGUCUUCAUUCUC | |
| AD-2005485 | csasuucuCfaUfUfGfucuucacauuL96 | | asAfsuguGfaAfGfacaaUfgAfgaaugsasa | | UUCAUUCUCAUUGUCUUCACAUU | |
| AD-2005527 | gsgsucuuGfcAfGfUfcuugucuuauL96 | | asUfsaagAfcAfAfgacuGfcAfagaccsasa | | UUGGUCUUGCAGUCUUGUCUUAG | |
| AD-2005530 | asasagaaUfaUfUfCfauguauaaguL96 | | asCfsuuaUfaCfAfugaaUfaUfucuuuscsc | | GGAAAGAAUAUUCAUGUAUAAGU | |
| AD-2005557 | ususgcaaUfuCfAfAfgcccuuguuuL96 | | asAfsacaAfgGfGfcuugAfaUfugcaasgsg | | CCUUGCAAUUCAAGCCCUUGUUG | |
| AD-2005575 | ususguucAfaGfGfGfucaacuguauL96 | | asUfsacaGfuUfGfacccUfuGfaacaascsa | | UGUUGUUCAAGGGUCAACUGUAA | |
| AD-2005588 | asascuguAfaUfAfGfgauauagcuuL96 | | asAfsgcuAfuAfUfccuaUfuAfcaguusgsa | | UCAACUGUAAUAGGAUAUAGCUA | |
| AD-2005604 | csusauuuUfuCfUfUfccucuaucauL96 | | asUfsgauAfgAfGfgaagAfaAfaauagscsu | | AGCUAUUUUUCUUCCUCUAUCAA | |
| AD-2005617 | uscsuaucAfaCfCfAfaaugguaaguL96 | | asCfsuuaCfcAfUfuuggUfuGfauagasgsg | | CCUCUAUCAACCAAAUGGUAAGC | |
| AD-2005638 | asuscuauUfuUfGfCfaguccacucuL96 | | asGfsaguGfgAfCfugcaAfaAfuagausgsc | | GCAUCUAUUUUGCAGUCCACUCU | |
| AD-2005658 | usascugaGfcUfAfAfauuauagauuL96 | | asAfsucuAfuAfAfuuuaGfcUfcaguasgsa | | UCUACUGAGCUAAAUUAUAGAUC | |
| AD-2005678 | cscsagcuAfuGfCfUfauuuauaauuL96 | | asAfsuuaUfaAfAfuagcAfuAfgcuggsasu | | AUCCAGCUAUGCUAUUUAUAAUU | |
| AD-2005687 | ususuucaAfuUfUfCfuccucugacuL96 | | asGfsucaGfaGfGfagaaAfuUfgaaaasusu | | AAUUUUCAAUUUCUCCUCUGACC | |
| AD-2005704 | gsasccauUfuCfAfGfaacaucuucuL96 | | asGfsaagAfuGfUfucugAfaAfuggucsasg | | CUGACCAUUUCAGAACAUCUUCC | |
| AD-2005720 | csusuccaAfuAfAfCfucauaaaacuL96 | | asGfsuuuUfaUfGfaguuAfuUfggaagsasu | | AUCUUCCAAUAACUCAUAAAACA | |
| AD-2005737 | csasacugAfaGfUfAfaaauugaguuL96 | | asAfscucAfaUfUfuuacUfuCfaguugsusu | | AACAACUGAAGUAAAAUUGAGUG | |
| AD-2005760 | gsgsaaaaUfaUfAfUfucaccaaacuL96 | | asGfsuuuGfgUfGfaauaUfaUfuuuccsasg | | CUGGAAAAUAUAUUCACCAAACU | |
| AD-2005826 | asasacuuUfgGfUfAfauuuaaguuuL96 | | asAfsacuUfaAfAfuuacCfaAfaguuusgsg | | CCAAACUUUGGUAAUUUAAGUUG | |
| AD-2005849 | asasaauaGfuUfUfAfcaccuauacuL96 | | asGfsuauAfgGfUfguaaAfcUfauuuusasg | | CUAAAAUAGUUUACACCUAUACU | |
| AD-2005862 | cscsuauaCfuGfCfAfuaauccaacuL96 | | asGfsuugGfaUfUfaugcAfgUfauaggsusg | | CACCUAUACUGCAUAAUCCAACA | |
| AD-2005923 | asascaauUfuUfAfAfuuucaguuguL96 | | asCfsaacUfgAfAfauuaAfaAfuuguusgsg | | CCAACAAUUUUAAUUUCAGUUGA | |
| AD-2005942 | asgsacauGfuUfAfCfuaauauaacuL96 | | asGfsuuaUfaUfUfaguaAfcAfugucususc | | GAAGACAUGUUACUAAUAUAACU | |
| AD-2005954 | asasagagUfaGfAfGfgauguguaauL96 | | asUfsuacAfcAfUfccucUfaCfucuuususa | | UAAAAGAGUAGAGGAUGUGUAAU | |
| AD-2006018 | gsusguaaUfuAfAfCfcauaucuucuL96 | | asGfsaagAfuAfUfgguuAfaUfuacacsasu | | AUGUGUAAUUAACCAUAUCUUCU | |
| AD-2006035 | ususcuaaAfaCfAfUfgguuacuaauL96 | | asUfsuagUfaAfCfcaugUfuUfuagaasgsa | | UCUUCUAAAACAUGGUUACUAAA | |
| AD-2006046 | ususacuaAfaAfGfAfauauguaacuL96 | | asGfsuuaCfaUfAfuucuUfuUfaguaascsc | | GGUUACUAAAAGAAUAUGUAACA | |
| AD-2006056 | asusguaaCfaUfCfAfauauugaccuL96 | | asGfsgucAfaUfAfuugaUfgUfuacausasu | | AUAUGUAACAUCAAUAUUGACCU | |
| AD-2006119 | asusugacCfuUfGfGfuuucuuacauL96 | | asUfsguaAfgAfAfaccaAfgGfucaausasu | | AUAUUGACCUUGGUUUCUUACAC | |
| AD-2006132 | uscsuuacAfcAfAfGfuguugcuaauL96 | | asUfsuagCfaAfCfacuuGfuGfuaagasasa | | UUUCUUACACAAGUGUUGCUAAC | |
| AD-2006150 | asascucaAfuAfGfUfgaaggagacuL96 | | asGfsucuCfcUfUfcacuAfuUfgaguusasg | | CUAACUCAAUAGUGAAGGAGACA | |
| AD-2006164 | gsgsagacAfcUfAfUfuaaauuuucuL96 | | asGfsaaaAfuUfUfaauaGfuGfucuccsusu | | AAGGAGACACUAUUAAAUUUUCU | |
| AD-2006234 | gsasacccAfuGfAfGfagauacuaguL96 | | asCfsuagUfaUfCfucucAfuGfgguucsasg | | CUGAACCCAUGAGAGAUACUAGA | |
| AD-2006242 | asgsuggaAfaGfUfGfuuugguucauL96 | | asUfsgaaCfcAfAfacacUfuUfccacuscsc | | GGAGUGGAAAGUGUUUGGUUCAG | |
| AD-2006259 | uscsagggAfuAfUfCfugaagaacauL96 | | asUfsguuCfuUfCfagauAfuCfccugasasc | | GUUCAGGGAUAUCUGAAGAACAG | |
| AD-2006334 | gscsagagAfuUfUfCfuuaagugacuL96 | | asGfsucaCfuUfAfagaaAfuCfucugcscsc | | GGGCAGAGAUUUCUUAAGUGACG | |
| AD-2006347 | asasgugaCfgCfCfUfcaucuacaauL96 | | asUfsuguAfgAfUfgaggCfgUfcacuusasa | | UUAAGUGACGCCUCAUCUACAAG | |
| AD-2006362 | usascaagCfuGfGfAfaauuccuaauL96 | | asUfsuagGfaAfUfuuccAfgCfuuguasgsa | | UCUACAAGCUGGAAAUUCCUAAA | |
| AD-2006437 | asgsuagaAfaGfCfUfuauaaacaauL96 | | asUfsuguUfuAfUfaagcUfuUfcuacususg | | CAAGUAGAAAGCUUAUAAACAAC | |
| AD-2006451 | asasacaaCfaGfGfUfgauacacucuL96 | | asGfsaguGfuAfUfcaccUfgUfuguuusasu | | AUAAACAACAGGUGAUACACUCA | |
| AD-2006525 | csascuggUfuUfUfAfguaaauuacuL96 | | asGfsuaaUfuUfAfcuaaAfaCfcagugsasg | | CUCACUGGUUUUAGUAAAUUACC | |
| AD-2006548 | csasgaaaGfuAfUfCfccuagucuuuL96 | | asAfsagaCfuAfGfggauAfcUfuucugsusa | | UACAGAAAGUAUCCCUAGUCUUA | |
| AD-2006626 | gsusggaaAfaUfUfUfgaacugauuuL96 | | asAfsaucAfgUfUfcaaaUfuUfuccacsusu | | AAGUGGAAAAUUUGAACUGAUUA | |
| AD-2006640 | csusgauuAfgUfCfAfuauuccuuuuL96 | | asAfsaagGfaAfUfaugaCfuAfaucagsusu | | AACUGAUUAGUCAUAUUCCUUUG | |
| AD-2006658 | ususgauuAfcAfCfUfguuuguuacuL96 | | asGfsuaaCfaAfAfcaguGfuAfaucaasasg | | CUUUGAUUACACUGUUUGUUACA | |
| AD-2006731 | uscsucagUfaAfAfCfagaaauaacuL96 | | asGfsuuaUfuUfCfuguuUfaCfugagasasa | | UUUCUCAGUAAACAGAAAUAACU | |
| AD-2006743 | uscsuucaUfuCfUfUfugauagaaauL96 | | asUfsuucUfaUfCfaaagAfaUfgaagasasc | | GUUCUUCAUUCUUUGAUAGAAAU | |
| AD-2006751 | asasaucuUfaUfUfCfugugaggauuL96 | | asAfsuccUfcAfCfagaaUfaAfgauuususa | | UAAAAUCUUAUUCUGUGAGGAUU | |
| AD-2006764 | usgsaggaUfuAfCfAfgaauacuauuL96 | | asAfsuagUfaUfUfcuguAfaUfccucascsa | | UGUGAGGAUUACAGAAUACUAUA | |
| AD-2006839 | csusucauCfaUfAfAfaguguaaaguL96 | | asCfsuuuAfcAfCfuuuaUfgAfugaagsasa | | UUCUUCAUCAUAAAGUGUAAAGA | |
| AD-2006862 | asusgcucAfaAfUfAfuguucuacuuL96 | | asAfsguaGfaAfCfauauUfuGfagcaususu | | AAAUGCUCAAAUAUGUUCUACUA | |
| AD-2006926 | uscsuacuAfuAfGfAfauaaguucuuL96 | | asAfsgaaCfuUfAfuucuAfuAfguagasasc | | GUUCUACUAUAGAAUAAGUUCUU | |
| AD-2006935 | ususcuuaUfcUfUfAfauuuacagguL96 | | asCfscugUfaAfAfuuaaGfaUfaagaascsu | | AGUUCUUAUCUUAAUUUACAGGG | |
| AD-2006965 | ususaaugUfuGfCfCfuuuuauauuuL96 | | asAfsauaUfaAfAfaggcAfaCfauuaasgsc | | GCUUAAUGUUGCCUUUUAUAUUU | |
| AD-2007024 | asasaucaGfaAfUfCfacuauauuauL96 | | asUfsaauAfuAfGfugauUfcUfgauuusgsc | | GCAAAUCAGAAUCACUAUAUUAA | |

TABLE 41
Unmodified Sense and Antisense Strand Sequences of CAMK2D dsRNA Agents Comprising an Unsaturated
C22 Hydrocarbon Chain Conjugated to Position 6 on the Sense Strand, Counting from the 5′-end of
the Sense Strand

| | | SEQ | Range in | | | SEQ | Range in | |
|---|---|---|---|---|---|---|---|---|
| Duplex | Sense Strand Sequence | ID | NM_001321571.2 | | Antisense Strand Sequence | ID | NM_001321571.2 | |
| Name | 5′ to 3′ | NO. | Start | End | 5′ to 3′ | NO. | Start | End |
| AD-2011305 | UAGCACUUUUGAUCAUCUUCU | | 3221 | 3241 | AGAAGAUGAUCAAAAGUGCUAGU | | 3219 | 3241 |
| AD-2011306 | CCUCAUCAAUAAAAUGCUUAU | | 1411 | 1431 | AUAAGCAUUUUAUUGAUGAGGUC | | 1409 | 1431 |
| AD-2011307 | UCCAUCAUUGUUAUUUUAACU | | 3763 | 3783 | AGUUAAAAUAACAAUGAUGGAGA | | 3761 | 3783 |
| AD-2011308 | UCCACACUAUUAUUCUAAACU | | 2001 | 2021 | AGUUUAGAAUAAUAGUGUGGAUU | | 1999 | 2021 |
| AD-2011309 | CUCCUAAAAGCUUCUCUAAAU | | 4614 | 4634 | AUUUAGAGAAGCUUUUAGGAGUC | | 4612 | 4634 |
| AD-2011310 | GGUCUCAUUUAGUACAUAACU | | 4491 | 4511 | AGUUAUGUACUAAAUGAGACCAG | | 4489 | 4511 |
| AD-2011311 | CAGACUUAAGAACUAUUGUUU | | 2620 | 2640 | AAACAAUAGUUCUUAAGUCUGUU | | 2618 | 2640 |
| AD-2011312 | CAUCAAGUUUCUCUGUUAAUU | | 2588 | 2608 | AAUUAACAGAGAAACUUGAUGAA | | 2586 | 2608 |
| AD-2011313 | AUUCUCUAUAUUCUACUUGUU | | 1277 | 1297 | AACAAGUAGAAUAUAGAGAAUGA | | 1275 | 1297 |
| AD-2011314 | CUGCUUGAAGAAAUUUAAUGU | | 1537 | 1557 | ACAUUAAAUUUCUUCAAGCAGUC | | 1535 | 1557 |
| AD-2011315 | CACUGACAUUAAUAUUCCUAU | | 5434 | 5454 | AUAGGAAUAUUAAUGUCAGUGAU | | 5432 | 5454 |
| AD-2011316 | AACCACAUUGUUUUGAAAAUU | | 5033 | 5053 | AAUUUUCAAAACAAUGUGGUUGA | | 5031 | 5053 |
| AD-2011317 | UCAUCACUAGCUAGUUUUCUU | | 3492 | 3512 | AAGAAAACUAGCUAGUGAUGAUG | | 3490 | 3512 |
| AD-2011318 | UUACUUUUUCCUAAGAUUCAU | | 4912 | 4932 | AUGAAUCUUAGGAAAAAGUAAGA | | 4910 | 4932 |
| AD-2011319 | UUACCUAGAAUAAAUAGUCUU | | 4866 | 4886 | AAGACUAUUUAUUCUAGGUAAGA | | 4864 | 4886 |
| AD-2011320 | GGUCUUCUAAAUUUCAACAGU | | 2296 | 2316 | ACUGUUGAAAUUUAGAAGACCCA | | 2294 | 2316 |
| AD-2011321 | CCUAUACAUUGUUUACACUUU | | 2466 | 2486 | AAAGUGUAAACAAUGUAUAGGAA | | 2464 | 2486 |
| AD-2011322 | UCUCUUGAAUCAGAGUAUCAU | | 3594 | 3614 | AUGAUACUCUGAUUCAAGAGAGA | | 3592 | 3614 |
| AD-2011323 | GCUGAGAACAAUAUAGUUAAU | | 4585 | 4605 | AUUAACUAUAUUGUUCUCAGCAA | | 4583 | 4605 |
| AD-2011324 | CGUCCAUCUUUAUUUCUUCAU | | 4407 | 4427 | AUGAAGAAAUAAAGAUGGACGCA | | 4405 | 4427 |
| AD-2011325 | AGCUUCUUUUGAUUUGCUAAU | | 4792 | 4812 | AUUAGCAAAUCAAAAGAAGCUGA | | 4790 | 4812 |
| AD-2011326 | AUCUCUUUUCUUUAUCCUGUU | | 3028 | 3048 | AACAGGAUAAAGAAAAGAGAUCA | | 3026 | 3048 |
| AD-2011327 | CACAAAUGAAUUACACAUUUU | | 4261 | 4281 | AAAAUGUGUAAUUCAUUUGUGGU | | 4259 | 4281 |
| AD-2011328 | UUUGUCCAAAAGCAAUAAACU | | 1978 | 1998 | AGUUUAUUGCUUUUGGACAAAGC | | 1976 | 1998 |
| AD-2011329 | UCUGAGUUUAAUCACUUUAGU | | 2945 | 2965 | ACUAAAGUGAUUAAACUCAGAAA | | 2943 | 2965 |
| AD-2011330 | CCAAGUACAACUCUUCAUCAU | | 2573 | 2593 | AUGAUGAAGAGUUGUACUUGGAA | | 2571 | 2593 |
| AD-2011331 | UUCUACUUUGAAAAUGCUUUU | | 1961 | 1981 | AAAAGCAUUUUCAAAGUAGAAUC | | 1959 | 1981 |
| AD-2011332 | UAUCAUAUAUUCCUUCCUAUU | | 2451 | 2471 | AAUAGGAAGGAAUAUAUGAUAAG | | 2449 | 2471 |
| AD-2011333 | CCUCUCAAAACCUACAUAAUU | | 4990 | 5010 | AAUUAUGUAGGUUUUGAGAGGGA | | 4988 | 5010 |
| AD-2011334 | AGUUAAUUGUUCUCUGUGAGU | | 4535 | 4555 | ACUCACAGAGAACAAUUAACUUG | | 4533 | 4555 |
| AD-2011335 | UUCUAUCAUUUUGAAACACUU | | 4287 | 4307 | AAGUGUUUCAAAAUGAUAGAAGA | | 4285 | 4307 |
| AD-2011336 | CAGCUUGAACUUGAGCAUACU | | 4762 | 4782 | AGUAUGCUCAAGUUCAAGCUGGG | | 4760 | 4782 |
| AD-2011337 | UCACCCAAAUUUUGAAUUUUU | | 5678 | 5698 | AAAAAUUCAAAAUUUGGGUGAAG | | 5676 | 5698 |
| AD-2011338 | GCAGUAUAACUAUUCUGAUCU | | 5415 | 5435 | AGAUCAGAAUAGUUAUACUGCUA | | 5413 | 5435 |
| AD-2011339 | GGUUUUCUUUGUGCUUCUCAU | | 3672 | 3692 | AUGAGAAGCACAAAGAAAACCUA | | 3670 | 3692 |
| AD-2011340 | AUGAGAAUAAACAUACCAACU | | 5324 | 5344 | AGUUGGUAUGUUUAUUCUCAUUU | | 5322 | 5344 |
| AD-2011341 | GAGUGAUUUCAUUGAAUAAAU | | 5746 | 5766 | AUUUAUUCAAUGAAAUCACUCGA | | 5744 | 5766 |
| AD-2011342 | GUUUCCAUAUUUGUAAAAUGU | | 5307 | 5327 | ACAUUUUACAAAUAUGGAAACUG | | 5305 | 5327 |
| AD-2011343 | CAUGCUGUUUUUGGUCAAACU | | 2897 | 2917 | AGUUUGACCAAAAACAGCAUGGC | | 2895 | 2917 |
| AD-2011344 | CUCUACUGCAGUAUAAUGUCU | | 5072 | 5092 | AGACAUUAUACUGCAGUAGAGGA | | 5070 | 5092 |
| AD-2011345 | UUGGUGUUUGAUUUAGUUACU | | 932 | 952 | AGUAACUAAAUCAAACACCAAGU | | 930 | 952 |
| AD-2011346 | UCUUUCUCCAGAAGUUUUACU | | 1210 | 1230 | AGUAAAACUUCUGGAGAAAGAUA | | 1208 | 1230 |
| AD-2011347 | AGAGUUCAAAUACAACAAUUU | | 1767 | 1787 | AAAUUGUUGUAUUUGAACUCUCA | | 1765 | 1787 |
| AD-2011348 | UCUGUGUUGAUUACUAAUCAU | | 3885 | 3905 | AUGAUUAGUAAUCAACACAGAGA | | 3883 | 3905 |
| AD-2011349 | UGCUUUUUUAGCUUCUCUCUU | | 3579 | 3599 | AAGAGAGAAGCUAAAAAAGCACA | | 3577 | 3599 |
| AD-2011350 | CUGUGACUGCAACGUCUUACU | | 3371 | 3391 | AGUAAGACGUUGCAGUCACAGGA | | 3369 | 3391 |
| AD-2011351 | CACUUCUGCAUUCUCUGUUCU | | 2319 | 2339 | AGAACAGAGAAUGCAGAAGUGGC | | 2317 | 2339 |
| AD-2011352 | CUCAUUUUUCCUUUGUUGAUU | | 3688 | 3708 | AAUCAACAAAGGAAAAAUGAGAA | | 3686 | 3708 |
| AD-2011353 | CCUCAGAAGCCUAUUUUUAAU | | 3799 | 3819 | AUUAAAAAUAGGCUUCUGAGGAC | | 3797 | 3819 |
| AD-2011354 | AUAGUAAUUCACAGUCCUCAU | | 3784 | 3804 | AUGAGGACUGUGAAUUACUAUCG | | 3782 | 3804 |
| AD-2011355 | UUUCUCAAAUCUGUGAUCUCU | | 3013 | 3033 | AGAGAUCACAGAUUUGAGAAAGA | | 3011 | 3033 |
| AD-2011356 | GCUGCCAAAAUUAUCAACACU | | 791 | 811 | AGUGUUGAUAAUUUUGGCAGCAU | | 789 | 811 |
| AD-2011357 | GACUGAUUGAUAUAUUUAAGU | | 4355 | 4375 | ACUUAAAUAUAUCAAUCAGUCCU | | 4353 | 4375 |
| AD-2011358 | UACACUUUUAACUUAGCAUGU | | 4164 | 4184 | ACAUGCUAAGUUAAAAGUGUAAA | | 4162 | 4184 |
| AD-2011359 | UCUGAGCAAGUUACCUCUUCU | | 5274 | 5294 | AGAAGAGGUAACUUGCUCAGAGC | | 5272 | 5294 |
| AD-2011360 | GCCCUCUUUAUGUAGGUUUAU | | 5508 | 5528 | AUAAACCUACAUAAAGAGGGCAG | | 5506 | 5528 |
| AD-2011361 | CAAGGAAUAUUAAUCUUCACU | | 5662 | 5682 | AGUGAAGAUUAAUAUUCCUUGAA | | 5660 | 5682 |
| AD-2011362 | AUGUUGUUUAUGUUUCUUACU | | 4850 | 4870 | AGUAAGAAACAUAAACAACAUGA | | 4848 | 4870 |
| AD-2011363 | AUACUCGGACUCAAAUGUCUU | | 5545 | 5565 | AAGACAUUUGAGUCCGAGUAUAU | | 5543 | 5565 |
| AD-2011364 | UGAAGCUUUUCUGAUAAUUAU | | 3944 | 3964 | AUAAUUAUCAGAAAAGCUUCAAC | | 3942 | 3964 |
| AD-2011365 | GAACUCUUUGCUGUUAAUCUU | | 2678 | 2698 | AAGAUUAACAGCAAAGAGUUCUA | | 2676 | 2698 |
| AD-2011366 | UUGAAAGUUUUGCUGAUUAAU | | 2796 | 2816 | AUUAAUCAGCAAAACUUUCAAUG | | 2794 | 2816 |
| AD-2011367 | AUGCUAGAAGAAAACUAAAGU | | 1554 | 1574 | ACUUUAGUUUUCUUCUAGCAUUA | | 1552 | 1574 |
| AD-2011368 | CCUCUUCAUGCAUGUUUCUGU | | 2378 | 2398 | ACAGAAACAUGCAUGAAGAGGAG | | 2376 | 2398 |
| AD-2011369 | UACUGCUUUUGAACCUGAAGU | | 1903 | 1923 | ACUUCAGGUUCAAAAGCAGUAAG | | 1901 | 1923 |
| AD-2011370 | CAACGUUCUACUGUUGCUUCU | | 1490 | 1510 | AGAAGCAACAGUAGAACGUUGAC | | 1488 | 1510 |
| AD-2011371 | CAGAUAGUCCUUGUUUUAAUU | | 5225 | 5245 | AAUUAAAACAAGGACUAUCUGGG | | 5223 | 5245 |
| AD-2011372 | ACACUGUAUAUAUUUCUUGCU | | 4322 | 4342 | AGCAAGAAAUAUAUACAGUGUCC | | 4320 | 4342 |
| AD-2011373 | AACUUAAUAAAGAUAUUGUGU | | 5341 | 5361 | ACACAAUAUCUUUAUUAAGUUGG | | 5339 | 5361 |
| AD-2011374 | UUUCUAUUUGUGUCUCCUCUU | | 5056 | 5076 | AAGAGGAGACACAAAUAGAAAUA | | 5054 | 5076 |
| AD-2011375 | GCUUACAAAUGUUUGCCAUUU | | 3524 | 3544 | AAAUGGCAAACAUUUGUAAGCCU | | 3522 | 3544 |
| AD-2011376 | CUGUGAUUUUUGUUCUCUUCU | | 3715 | 3735 | AGAAGAGAACAAAAAUCACAGGA | | 3713 | 3735 |
| AD-2011377 | UGAUCGAAGCUAUCAACAAUU | | 1842 | 1862 | AAUUGUUGAUAGCUUCGAUCAGU | | 1840 | 1862 |
| AD-2011378 | AUGCCUGUUUAUGCUGUUCAU | | 4831 | 4851 | AUGAACAGCAUAAACAGGCAUGU | | 4829 | 4851 |
| AD-2011379 | AGAAUUUCCCAGUUUAAAACU | | 3634 | 3654 | AGUUUUAAACUGGGAAAUUCUAA | | 3632 | 3654 |
| AD-2011380 | GUAGAUCAAGUUUGUCUUCCU | | 4202 | 4222 | AGGAAGACAAACUUGAUCUACAG | | 4200 | 4222 |
| AD-2011381 | ACAACCCUUCCUCUUUUCUCU | | 3853 | 3873 | AGAGAAAAGAGGAAGGGUUGUUU | | 3851 | 3873 |
| AD-2011382 | AGUGAAAUUUGCAUAAUGAAU | | 3162 | 3182 | AUUCAUUAUGCAAAUUUCACUUC | | 3160 | 3182 |
| AD-2011383 | UUAGGAUAUAUUCACAUUGUU | | 2961 | 2981 | AACAAUGUGAAUAUAUCCUAAAG | | 2959 | 2981 |
| AD-2011384 | UCUUAGAUAUUAUUGCUAGUU | | 3905 | 3925 | AACUAGCAAUAAUAUCUAAGAUG | | 3903 | 3925 |
| AD-2011385 | CUGGUUAAAAUGGAUGAUUUU | | 2772 | 2792 | AAAAUCAUCCAUUUUAACCAGGA | | 2770 | 2792 |
| AD-2011386 | CUUCCCUGAAGUGCUUUACAU | | 3731 | 3751 | AUGUAAAGCACUUCAGGGAAGAG | | 3729 | 3751 |
| AD-2011387 | CACCACUGUCUUGAUGCUCUU | | 5257 | 5277 | AAGAGCAUCAAGACAGUGGUGAG | | 5255 | 5277 |
| AD-2011388 | ACAACUCUUGUUUUGCUGUUU | | 5483 | 5503 | AAACAGCAAAACAAGAGUUGUAU | | 5481 | 5503 |
| AD-2011389 | CCUUUCGUUUGCUUUCUUAUU | | 3057 | 3077 | AAUAAGAAAGCAAACGAAAGGAA | | 3055 | 3077 |
| AD-2011390 | GGGCAAAAUCACUUAUGAAAU | | 2838 | 2858 | AUUUCAUAAGUGAUUUUGCCCAC | | 2836 | 2858 |
| AD-2011391 | AACACUUUGAUGUUAUCAUUU | | 4703 | 4723 | AAAUGAUAACAUCAAAGUGUUAC | | 4701 | 4723 |
| AD-2011392 | UUCCUCCAAGGAGUUAGAAUU | | 3619 | 3639 | AAUUCUAACUCCUUGGAGGAAGA | | 3617 | 3639 |
| AD-2011393 | AAGUGUUUUGAACUUGAUCUU | | 3549 | 3569 | AAGAUCAAGUUCAAAACACUUAU | | 3547 | 3569 |
| AD-2011394 | UGGCCUCAUUUUUCUCUUUUU | | 4060 | 4080 | AAAAAGAGAAAAAUGAGGCCAUU | | 4058 | 4080 |
| AD-2011395 | AGGUAUUUGCAUUGUUUAAAU | | 3328 | 3348 | AUUUAAACAAUGCAAAUACCUCC | | 3326 | 3348 |
| AD-2011396 | CCUAAUAUUGUGCGACUUCAU | | 881 | 901 | AUGAAGUCGCACAAUAUUAGGGU | | 879 | 901 |
| AD-2011397 | ACACCAAAUGAAGUGGUCAUU | | 5582 | 5602 | AAUGACCACUUCAUUUGGUGUCU | | 5580 | 5602 |
| AD-2011398 | GUAGCAAAUCAGUAUAUUCUU | | 5631 | 5651 | AAGAAUAUACUGAUUUGCUACUA | | 5629 | 5651 |
| AD-2011399 | CGAAAGCAAGAGAUUAUCAAU | | 1808 | 1828 | AUUGAUAAUCUCUUGCUUUCGUG | | 1806 | 1828 |
| AD-2011400 | CCAACACAGACUCUAUCAGCU | | 1324 | 1344 | AGCUGAUAGAGUCUGUGUUGGUC | | 1322 | 1344 |
| AD-2011401 | GACUAGAAUGAUAUUUGAUAU | | 5381 | 5401 | AUAUCAAAUAUCAUUCUAGUCAC | | 5379 | 5401 |
| AD-2011402 | GCCUGAGAAUUUGCUUUUAGU | | 1084 | 1104 | ACUAAAAGCAAAUUCUCAGGCUU | | 1082 | 1104 |
| AD-2011403 | CAAAACAAUGGUCUCUUCUGU | | 4555 | 4575 | ACAGAAGAGACCAUUGUUUUGCU | | 4553 | 4575 |
| AD-2011404 | GAUAAACAACAAAGCCAACGU | | 1654 | 1674 | ACGUUGGCUUUGUUGUUUAUCUU | | 1652 | 1674 |
| AD-2011405 | UUUUUUAAUACGAACCUGUCU | | 4678 | 4698 | AGACAGGUUCGUAUUAAAAAAAA | | 4676 | 4698 |
| AD-2011406 | GGUUUAUAAUUCUGCUUAAGU | | 3273 | 3293 | ACUUAAGCAGAAUUAUAAACCUU | | 3271 | 3293 |
| AD-2011407 | AGGAUAAAAGCAAGUUCCUCU | | 3351 | 3371 | AGAGGAACUUGCUUUUAUCCUAA | | 3349 | 3371 |
| AD-2011408 | UGUAGAGUGUUUUUUUUACAU | | 4148 | 4168 | AUGUAAAAAAAACACUCUACAUC | | 4146 | 4168 |
| AD-2011409 | UUCUCUGAAUGACAGUUGUAU | | 2638 | 2658 | AUACAACUGUCAUUCAGAGAACA | | 2636 | 2658 |
| AD-2011410 | AUUGUAUUUACCUCUCCUGUU | | 5012 | 5032 | AACAGGAGAGGUAAAUACAAUCU | | 5010 | 5032 |
| AD-2011411 | UGGCAUACAUGUAUUUAAAGU | | 4115 | 4135 | ACUUUAAAUACAUGUAUGCCACG | | 4113 | 4135 |
| AD-2011412 | UUGUCAAGAUUUUAAAGGUUU | | 3257 | 3277 | AAACCUUUAAAAUCUUGACAACA | | 3255 | 3277 |
| AD-2011413 | UAUGAAAAUUCCUACUGGACU | | 763 | 783 | AGUCCAGUAGGAAUUUUCAUACA | | 761 | 783 |
| AD-2011414 | CCUCUUUGUGGCAAAACAUCU | | 2250 | 2270 | AGAUGUUUUGCCACAAAGAGGUG | | 2248 | 2270 |
| AD-2011415 | CAGUCAGAAGAGACUCGUGUU | | 2114 | 2134 | AACACGAGUCUCUUCUGACUGCA | | 2112 | 2134 |
| AD-2011416 | AAGUCACUGAACAACUGAUCU | | 1827 | 1847 | AGAUCAGUUGUUCAGUGACUUUG | | 1825 | 1847 |
| AD-2011417 | GGAGCUUAUGAUUUUCCAUCU | | 1355 | 1375 | AGAUGGAAAAUCAUAAGCUCCAG | | 1353 | 1375 |
| AD-2011418 | GUGCGGAUCGUUUCGCAACUU | | 278 | 298 | AAGUUGCGAAACGAUCCGCACUG | | 276 | 298 |
| AD-2011419 | AAUCUGCCGUCUUUUGAAGCU | | 859 | 879 | AGCUUCAAAAGACGGCAGAUUCU | | 857 | 879 |
| AD-2011420 | UACUUCUUUUCCCGACUUCUU | | 4893 | 4913 | AAGAAGUCGGGAAAAGAAGUAGG | | 4891 | 4913 |
| AD-2011421 | CUACUGUAAUCCACAACCCUU | | 1725 | 1745 | AAGGGUUGUGGAUUACAGUAGUU | | 1723 | 1745 |
| AD-2011422 | UUUGUUCCAUUCUUUUCUUAU | | 3083 | 3103 | AUAAGAAAAGAAUGGAACAAAAG | | 3081 | 3103 |
| AD-2011423 | GAAGAUGUGAAAGCACGAAAU | | 1793 | 1813 | AUUUCGUGCUUUCACAUCUUCAU | | 1791 | 1813 |
| AD-2011424 | CAGUAAAUAUAUUGAGCCAUU | | 4007 | 4027 | AAUGGCUCAAUAUAUUUACUGUA | | 4005 | 4027 |
| AD-2011425 | AAUGGGAAAGAAAACUUCUCU | | 2222 | 2242 | AGAGAAGUUUUCUUUCCCAUUUG | | 2220 | 2242 |
| AD-2011426 | GGCAGAAUGUUCAUUUUCAUU | | 2157 | 2177 | AAUGAAAAUGAACAUUCUGCCAC | | 2155 | 2177 |
| AD-2011427 | CGCCUGCAUAGCAUAUAUUAU | | 2050 | 2070 | AUAAUAUAUGCUAUGCAGGCGGC | | 2048 | 2070 |
| AD-2011428 | CUGGCUGUUUUUCCAUUUCCU | | 318 | 338 | AGGAAAUGGAAAAACAGCCAGGC | | 316 | 338 |
| AD-2011429 | AGGGAAAUAAAAGUCUUUUGU | | 3188 | 3208 | ACAAAAGACUUUUAUUUCCCUUU | | 3186 | 3208 |
| AD-2011430 | CCACCCUUUCUGGUCAUCUCU | | 481 | 501 | AGAGAUGACCAGAAAGGGUGGCG | | 479 | 501 |
| AD-2011431 | UGCCAUAAUGGUAAAGGACUU | | 4339 | 4359 | AAGUCCUUUACCAUUAUGGCAAG | | 4337 | 4359 |
| AD-2011432 | CAAACUGUGUAAACUGGAAAU | | 2912 | 2932 | AUUUCCAGUUUACACAGUUUGAC | | 2910 | 2932 |
| AD-2011433 | UAAACCCUCAUGUACAUCUGU | | 2016 | 2036 | ACAGAUGUACAUGAGGGUUUAGA | | 2014 | 2036 |
| AD-2011434 | UUUUGUGAAGCAGCUAUACGU | | 4095 | 4115 | ACGUAUAGCUGCUUCACAAAAGG | | 4093 | 4115 |
| AD-2011435 | AUGGAGAUAUCAUUGAUAAAU | | 5151 | 5171 | AUUUAUCAAUGAUAUCUCCAUUC | | 5149 | 5171 |
| AD-2011436 | GUGUAUUCAUCAUUGCAUUCU | | 5108 | 5128 | AGAAUGCAAUGAUGAAUACACAG | | 5106 | 5128 |
| AD-2011437 | GAAGCUUUGGGUAAUUUAGUU | | 1919 | 1939 | AACUAAAUUACCCAAAGCUUCAG | | 1917 | 1939 |
| AD-2011438 | GCUGUGAAAUUGGCAGACUUU | | 1124 | 1144 | AAAGUCUGCCAAUUUCACAGCUG | | 1122 | 1144 |
| AD-2011439 | UAACAAUUAAAGUGGGAUGAU | | 2813 | 2833 | AUCAUCCCACUUUAAUUGUUAAU | | 2811 | 2833 |
| AD-2011440 | GCUGGCUACAAGGAAUUUCUU | | 1594 | 1614 | AAGAAAUUCCUUGUAGCCAGCAU | | 1592 | 1614 |
| AD-2011441 | UGUUAUUAUUCUUUGCUCACU | | 5455 | 5475 | AGUGAGCAAAGAAUAAUAACAAU | | 5453 | 5475 |
| AD-2011442 | AUAACAAUUUGCACUUGGUGU | | 4506 | 4526 | ACACCAAGUGCAAAUUGUUAUGU | | 4504 | 4526 |
| AD-2011443 | CUUCCGCACUAAGAUGUGAGU | | 4217 | 4237 | ACUCACAUCUUAGUGCGGAAGAC | | 4215 | 4237 |
| AD-2011444 | CUCUCAUCUCACCUCUCUGUU | | 3870 | 3890 | AACAGAGAGGUGAGAUGAGAGAA | | 3868 | 3890 |
| AD-2011445 | CACCCAUGGAUCUGUCAACGU | | 1475 | 1495 | ACGUUGACAGAUCCAUGGGUGCU | | 1473 | 1495 |
| AD-2011446 | AUAAAAUUCUACUGACUUCUU | | 5761 | 5781 | AAGAAGUCAGUAGAAUUUUAUUC | | 5759 | 5781 |
| AD-2011447 | UGCCAUCUUGACAACUAUGCU | | 1576 | 1596 | AGCAUAGUUGUCAAGAUGGCACC | | 1574 | 1596 |
| AD-2011448 | ACUUAGGUAUCCUAACUAUGU | | 3304 | 3324 | ACAUAGUUAGGAUACCUAAGUCC | | 3302 | 3324 |
| AD-2011449 | CUGCUUCUACUCCUCCUGCUU | | 255 | 275 | AAGCAGGAGGAGUAGAAGCAGAG | | 253 | 275 |
| AD-2011450 | UGAGGAAAUUGUGAUUUGUUU | | 4233 | 4253 | AAACAAAUCACAAUUUCCUCACA | | 4231 | 4253 |
| AD-2011451 | GACGAGUAUCAGCUUUUCGAU | | 704 | 724 | AUCGAAAAGCUGAUACUCGUCCG | | 702 | 724 |
| AD-2011452 | UCCCUCUGACCCUCAGUUUCU | | 5292 | 5312 | AGAAACUGAGGGUCAGAGGGAAG | | 5290 | 5312 |
| AD-2011453 | GCCCUUUUGAUAACAGAAGCU | | 5192 | 5212 | AGCUUCUGUUAUCAAAAGGGCUU | | 5190 | 5212 |
| AD-2011454 | AGAAAUUAAAUGGUAGCAGUU | | 5400 | 5420 | AACUGCUACCAUUUAAUUUCUAU | | 5398 | 5420 |
| AD-2011455 | GAUGGAUUUUCACCGAUUCUU | | 1945 | 1965 | AAGAAUCGGUGAAAAUCCAUCCC | | 1943 | 1965 |
| AD-2011456 | ACAUCACACUUGCUCACAUGU | | 4814 | 4834 | ACAUGUGAGCAAGUGUGAUGUUU | | 4812 | 4834 |
| AD-2011457 | UCUAAGGCCUGAAAACCAUUU | | 2268 | 2288 | AAAUGGUUUUCAGGCCUUAGAUG | | 2266 | 2288 |
| AD-2011458 | UUACGUAAAGAUCCUUAUGGU | | 1226 | 1246 | ACCAUAAGGAUCUUUACGUAAAA | | 1224 | 1246 |
| AD-2011459 | UACAGUGAAGCUGAUGCCAGU | | 992 | 1012 | ACUGGCAUCAGCUUCACUGUAGU | | 990 | 1012 |
| AD-2011460 | AUUUGCUGAAUUGAAUUGUUU | | 2989 | 3009 | AAACAAUUCAAUUCAGCAAAUUC | | 2987 | 3009 |
| AD-2011461 | UAAGCUCUAGUUUGGACUUAU | | 3289 | 3309 | AUAAGUCCAAACUAGAGCUUAAG | | 3287 | 3309 |
| AD-2011462 | CUGGCUAGUAGUGUGUGAGAU | | 3132 | 3152 | AUCUCACACACUACUAGCCAGCC | | 3130 | 3152 |
| AD-2011463 | AUGUGAUGCAUCAUCUUAUCU | | 2435 | 2455 | AGAUAAGAUGAUGCAUCACAUAU | | 2433 | 2455 |
| AD-2011464 | CCUACAUGUAAUGCAUAUGUU | | 2419 | 2439 | AACAUAUGCAUUACAUGUAGGAC | | 2417 | 2439 |
| AD-2011465 | CAGCAGCCAAGAGUUUGUUGU | | 1614 | 1634 | ACAACAAACUCUUGGCUGCUGAG | | 1612 | 1634 |
| AD-2011466 | UCCAAUCCAGCCUUCACAUGU | | 4642 | 4662 | ACAUGUGAAGGCUGGAUUGGAGG | | 4640 | 4662 |
| AD-2011467 | ACCAACAGUACCCAUCAAGCU | | 2188 | 2208 | AGCUUGAUGGGUACUGUUGGUGA | | 2186 | 2208 |
| AD-2011468 | CUGCUAGGGAUCAUCAGAAAU | | 822 | 842 | AUUUCUGAUGAUCCCUAGCAGAA | | 820 | 842 |
| AD-2011469 | GGUUUGCUACCACAUAAAGCU | | 2877 | 2897 | AGCUUUAUGUGGUAGCAAACCAA | | 2875 | 2897 |
| AD-2011470 | AGUGUACAGCAUCAUGCUCCU | | 4931 | 4951 | AGGAGCAUGAUGCUGUACACUGA | | 4929 | 4951 |
| AD-2011471 | UGGCUUCGACCACAACCUGCU | | 672 | 692 | AGCAGGUUGUGGUCGAAGCCAUC | | 670 | 692 |
| AD-2011472 | CCAGUCAUUGCAUUCAGCAGU | | 1008 | 1028 | ACUGCUGAAUGCAAUGACUGGCA | | 1006 | 1028 |
| AD-2011473 | CUUCCAUGAUGCACAGACAGU | | 1506 | 1526 | ACUGUCUGUGCAUCAUGGAAGCA | | 1504 | 1526 |
| AD-2011474 | CAGGAGGAUACCAACUUGAUU | | 3407 | 3427 | AAUCAAGUUGGUAUCCUCCUGGC | | 3405 | 3427 |
| AD-2011475 | UGAUGGAAACAAGGAGUCAAU | | 1744 | 1764 | AUUGACUCCUUGUUUCCAUCAGG | | 1742 | 1764 |
| AD-2011476 | AAGCAAGAAUCAGUUGGUUUU | | 2862 | 2882 | AAAACCAACUGAUUCUUGCUUCU | | 2860 | 2882 |
| AD-2011477 | CAACUGCUUGCCACUCGUCCU | | 293 | 313 | AGGACGAGUGGCAAGCAGUUGCG | | 291 | 313 |
| AD-2011478 | GUGUUGAAGUAUUACUGUAGU | | 4186 | 4206 | ACUACAGUAAUACUUCAACACCA | | 4184 | 4206 |
| AD-2011479 | UUACUGUUGGCAAAACAAUAU | | 2517 | 2537 | AUAUUGUUUUGCCAACAGUAAUU | | 2515 | 2537 |
| AD-2011480 | CAGUGUGUCCUACUCUGGUCU | | 4475 | 4495 | AGACCAGAGUAGGACACACUGCC | | 4473 | 4495 |
| AD-2011481 | AGAAACCAGAUGGAGUAAAGU | | 1635 | 1655 | ACUUUACUCCAUCUGGUUUCUUC | | 1633 | 1655 |
| AD-2011482 | GCUCCACAGCAAACCUUCCUU | | 4946 | 4966 | AAGGAAGGUUUGCUGUGGAGCAU | | 4944 | 4966 |
| AD-2011483 | GACUUUGAAGCCUACACAAAU | | 1865 | 1885 | AUUUGUGUAGGCUUCAAAGUCCC | | 1863 | 1885 |

TABLE 42
Modified Sense and Antisense Strand Sequences of CAMK2D dsRNA Agents Comprising an Unsaturated C22 Hydrocarbon Chain
Conjugated to Position 6 on the Sense Strand, Counting from the 5′-end of the Sense Strand

| | | SEQ | | SEQ | | SEQ |
|---|---|---|---|---|---|---|
| Duplex | | ID | | ID | | ID |
| Name | Sense Strand Sequence 5′ to 3′ | NO. | Antisense Strand Sequence 5′ to 3′ | NO. | mRNA target sequence | NO. |
| AD-2011304 | cscsaca(Cda)AfaAfCfUfuaaauuacuuL96 | | asAfsguaAfuUfUfaaguUfuGfuguggsasa | | UUCCACACAAACUUAAAUUACUG | |
| AD-2011305 | usasgca(Cda)UfuUfUfGfaucaucuucuL96 | | asGfsaagAfuGfAfucaaAfaGfugcuasgsu | | ACUAGCACUUUUGAUCAUCUUCA | |
| AD-2011306 | cscsuca(Uda)CfaAfUfAfaaaugcuuauL96 | | asUfsaagCfaUfUfuuauUfgAfugaggsusc | | GACCUCAUCAAUAAAAUGCUUAC | |
| AD-2011307 | uscscau(Cda)AfuUfGfUfuauuuuaacuL96 | | asGfsuuaAfaAfUfaacaAfuGfauggasgsa | | UCUCCAUCAUUGUUAUUUUAACG | |
| AD-2011308 | uscscac(Ada)CfuAfUfUfauucuaaacuL96 | | asGfsuuuAfgAfAfuaauAfgUfguggasusu | | AAUCCACACUAUUAUUCUAAACC | |
| AD-2011309 | csusccu(Ada)AfaAfGfCfuucucuaaauL96 | | asUfsuuaGfaGfAfagcuUfuUfaggagsusc | | GACUCCUAAAAGCUUCUCUAAAC | |
| AD-2011310 | gsgsucu(Cda)AfuUfUfAfguacauaacuL96 | | asGfsuuaUfgUfAfcuaaAfuGfagaccsasg | | CUGGUCUCAUUUAGUACAUAACA | |
| AD-2011311 | csasgac(Uda)UfaAfGfAfacuauuguuuL96 | | asAfsacaAfuAfGfuucuUfaAfgucugsusu | | AACAGACUUAAGAACUAUUGUUC | |
| AD-2011312 | csasuca(Ada)GfuUfUfCfucuguuaauuL96 | | asAfsuuaAfcAfGfagaaAfcUfugaugsasa | | UUCAUCAAGUUUCUCUGUUAAUG | |
| AD-2011313 | asusucu(Cda)UfaUfAfUfucuacuuguuL96 | | asAfscaaGfuAfGfaauaUfaGfagaausgsa | | UCAUUCUCUAUAUUCUACUUGUG | |
| AD-2011314 | csusgcu(Uda)GfaAfGfAfaauuuaauguL96 | | asCfsauuAfaAfUfuucuUfcAfagcagsusc | | GACUGCUUGAAGAAAUUUAAUGC | |
| AD-2011315 | csascug(Ada)CfaUfUfAfauauuccuauL96 | | asUfsaggAfaUfAfuuaaUfgUfcagugsasu | | AUCACUGACAUUAAUAUUCCUAU | |
| AD-2011316 | asascca(Cda)AfuUfGfUfuuugaaaauuL96 | | asAfsuuuUfcAfAfaacaAfuGfugguusgsa | | UCAACCACAUUGUUUUGAAAAUA | |
| AD-2011317 | uscsauc(Ada)CfuAfGfCfuaguuuucuuL96 | | asAfsgaaAfaCfUfagcuAfgUfgaugasusg | | CAUCAUCACUAGCUAGUUUUCUA | |
| AD-2011318 | ususacu(Uda)UfuUfCfCfuaagauucauL96 | | asUfsgaaUfcUfUfaggaAfaAfaguaasgsa | | UCUUACUUUUUCCUAAGAUUCAG | |
| AD-2011319 | ususacc(Uda)AfgAfAfUfaaauagucuuL96 | | asAfsgacUfaUfUfuauuCfuAfgguaasgsa | | UCUUACCUAGAAUAAAUAGUCUC | |
| AD-2011320 | gsgsucu(Uda)CfuAfAfAfuuucaacaguL96 | | asCfsuguUfgAfAfauuuAfgAfagaccscsa | | UGGGUCUUCUAAAUUUCAACAGU | |
| AD-2011321 | cscsuau(Ada)CfaUfUfGfuuuacacuuuL96 | | asAfsaguGfuAfAfacaaUfgUfauaggsasa | | UUCCUAUACAUUGUUUACACUUC | |
| AD-2011322 | uscsucu(Uda)GfaAfUfCfagaguaucauL96 | | asUfsgauAfcUfCfugauUfcAfagagasgsa | | UCUCUCUUGAAUCAGAGUAUCAU | |
| AD-2011323 | gscsuga(Gda)AfaCfAfAfuauaguuaauL96 | | asUfsuaaCfuAfUfauugUfuCfucagcsasa | | UUGCUGAGAACAAUAUAGUUAAC | |
| AD-2011324 | csgsucc(Ada)UfcUfUfUfauuucuucauL96 | | asUfsgaaGfaAfAfuaaaGfaUfggacgscsa | | UGCGUCCAUCUUUAUUUCUUCAG | |
| AD-2011325 | asgscuu(Cda)UfuUfUfGfauuugcuaauL96 | | asUfsuagCfaAfAfucaaAfaGfaagcusgsa | | UCAGCUUCUUUUGAUUUGCUAAA | |
| AD-2011326 | asuscuc(Uda)UfuUfCfUfuuauccuguuL96 | | asAfscagGfaUfAfaagaAfaAfgagauscsa | | UGAUCUCUUUUCUUUAUCCUGUU | |
| AD-2011327 | csascaa(Ada)UfgAfAfUfuacacauuuuL96 | | asAfsaauGfuGfUfaauuCfaUfuugugsgsu | | ACCACAAAUGAAUUACACAUUUA | |
| AD-2011328 | ususugu(Cda)CfaAfAfAfgcaauaaacuL96 | | asGfsuuuAfuUfGfcuuuUfgGfacaaasgsc | | GCUUUGUCCAAAAGCAAUAAACC | |
| AD-2011329 | uscsuga(Gda)UfuUfAfAfucacuuuaguL96 | | asCfsuaaAfgUfGfauuaAfaCfucagasasa | | UUUCUGAGUUUAAUCACUUUAGG | |
| AD-2011330 | cscsaag(Uda)AfcAfAfCfucuucaucauL96 | | asUfsgauGfaAfGfaguuGfuAfcuuggsasa | | UUCCAAGUACAACUCUUCAUCAA | |
| AD-2011331 | ususcua(Cda)UfuUfGfAfaaaugcuuuuL96 | | asAfsaagCfaUfUfuucaAfaGfuagaasusc | | GAUUCUACUUUGAAAAUGCUUUG | |
| AD-2011332 | usasuca(Uda)AfuAfUfUfccuuccuauuL96 | | asAfsuagGfaAfGfgaauAfuAfugauasasg | | CUUAUCAUAUAUUCCUUCCUAUA | |
| AD-2011333 | cscsucu(Cda)AfaAfAfCfcuacauaauuL96 | | asAfsuuaUfgUfAfgguuUfuGfagaggsgsa | | UCCCUCUCAAAACCUACAUAAUA | |
| AD-2011334 | asgsuua(Ada)UfuGfUfUfcucugugaguL96 | | asCfsucaCfaGfAfgaacAfaUfuaacususg | | CAAGUUAAUUGUUCUCUGUGAGC | |
| AD-2011335 | ususcua(Uda)CfaUfUfUfugaaacacuuL96 | | asAfsgugUfuUfCfaaaaUfgAfuagaasgsa | | UCUUCUAUCAUUUUGAAACACUG | |
| AD-2011336 | csasgcu(Uda)GfaAfCfUfugagcauacuL96 | | asGfsuauGfcUfCfaaguUfcAfagcugsgsg | | CCCAGCUUGAACUUGAGCAUACA | |
| AD-2011337 | uscsacc(Cda)AfaAfUfUfuugaauuuuuL96 | | asAfsaaaUfuCfAfaaauUfuGfggugasasg | | CUUCACCCAAAUUUUGAAUUUUU | |
| AD-2011338 | gscsagu(Ada)UfaAfCfUfauucugaucuL96 | | asGfsaucAfgAfAfuaguUfaUfacugcsusa | | UAGCAGUAUAACUAUUCUGAUCA | |
| AD-2011339 | gsgsuuu(Uda)CfuUfUfGfugcuucucauL96 | | asUfsgagAfaGfCfacaaAfgAfaaaccsusa | | UAGGUUUUCUUUGUGCUUCUCAU | |
| AD-2011340 | asusgag(Ada)AfuAfAfAfcauaccaacuL96 | | asGfsuugGfuAfUfguuuAfuUfcucaususu | | AAAUGAGAAUAAACAUACCAACU | |
| AD-2011341 | gsasgug(Ada)UfuUfCfAfuugaauaaauL96 | | asUfsuuaUfuCfAfaugaAfaUfcacucsgsa | | UCGAGUGAUUUCAUUGAAUAAAA | |
| AD-2011342 | gsusuuc(Cda)AfuAfUfUfuguaaaauguL96 | | asCfsauuUfuAfCfaaauAfuGfgaaacsusg | | CAGUUUCCAUAUUUGUAAAAUGA | |
| AD-2011343 | csasugc(Uda)GfuUfUfUfuggucaaacuL96 | | asGfsuuuGfaCfCfaaaaAfcAfgcaugsgsc | | GCCAUGCUGUUUUUGGUCAAACU | |
| AD-2011344 | csuscua(Cda)UfgCfAfGfuauaaugucuL96 | | asGfsacaUfuAfUfacugCfaGfuagagsgsa | | UCCUCUACUGCAGUAUAAUGUCU | |
| AD-2011345 | ususggu(Gda)UfuUfGfAfuuuaguuacuL96 | | asGfsuaaCfuAfAfaucaAfaCfaccaasgsu | | ACUUGGUGUUUGAUUUAGUUACU | |
| AD-2011346 | uscsuuu(Cda)UfcCfAfGfaaguuuuacuL96 | | asGfsuaaAfaCfUfucugGfaGfaaagasusa | | UAUCUUUCUCCAGAAGUUUUACG | |
| AD-2011347 | asgsagu(Uda)CfaAfAfUfacaacaauuuL96 | | asAfsauuGfuUfGfuauuUfgAfacucuscsa | | UGAGAGUUCAAAUACAACAAUUG | |
| AD-2011348 | uscsugu(Gda)UfuGfAfUfuacuaaucauL96 | | asUfsgauUfaGfUfaaucAfaCfacagasgsa | | UCUCUGUGUUGAUUACUAAUCAU | |
| AD-2011349 | usgscuu(Uda)UfuUfAfGfcuucucucuuL96 | | asAfsgagAfgAfAfgcuaAfaAfaagcascsa | | UGUGCUUUUUUAGCUUCUCUCUU | |
| AD-2011350 | csusgug(Ada)CfuGfCfAfacgucuuacuL96 | | asGfsuaaGfaCfGfuugcAfgUfcacagsgsa | | UCCUGUGACUGCAACGUCUUACU | |
| AD-2011351 | csascuu(Cda)UfgCfAfUfucucuguucuL96 | | asGfsaacAfgAfGfaaugCfaGfaagugsgsc | | GCCACUUCUGCAUUCUCUGUUCU | |
| AD-2011352 | csuscau(Uda)UfuUfCfCfuuuguugauuL96 | | asAfsucaAfcAfAfaggaAfaAfaugagsasa | | UUCUCAUUUUUCCUUUGUUGAUU | |
| AD-2011353 | cscsuca(Gda)AfaGfCfCfuauuuuuaauL96 | | asUfsuaaAfaAfUfaggcUfuCfugaggsasc | | GUCCUCAGAAGCCUAUUUUUAAA | |
| AD-2011354 | asusagu(Ada)AfuUfCfAfcaguccucauL96 | | asUfsgagGfaCfUfgugaAfuUfacuauscsg | | CGAUAGUAAUUCACAGUCCUCAG | |
| AD-2011355 | ususucu(Cda)AfaAfUfCfugugaucucuL96 | | asGfsagaUfcAfCfagauUfuGfagaaasgsa | | UCUUUCUCAAAUCUGUGAUCUCU | |
| AD-2011356 | gscsugc(Cda)AfaAfAfUfuaucaacacuL96 | | asGfsuguUfgAfUfaauuUfuGfgcagcsasu | | AUGCUGCCAAAAUUAUCAACACC | |
| AD-2011357 | gsascug(Ada)UfuGfAfUfauauuuaaguL96 | | asCfsuuaAfaUfAfuaucAfaUfcagucscsu | | AGGACUGAUUGAUAUAUUUAAGA | |
| AD-2011358 | usascac(Uda)UfuUfAfAfcuuagcauguL96 | | asCfsaugCfuAfAfguuaAfaAfguguasasa | | UUUACACUUUUAACUUAGCAUGU | |
| AD-2011359 | uscsuga(Gda)CfaAfGfUfuaccucuucuL96 | | asGfsaagAfgGfUfaacuUfgCfucagasgsc | | GCUCUGAGCAAGUUACCUCUUCC | |
| AD-2011360 | gscsccu(Cda)UfuUfAfUfguagguuuauL96 | | asUfsaaaCfcUfAfcauaAfaGfagggcsasg | | CUGCCCUCUUUAUGUAGGUUUAC | |
| AD-2011361 | csasagg(Ada)AfuAfUfUfaaucuucacuL96 | | asGfsugaAfgAfUfuaauAfuUfccuugsasa | | UUCAAGGAAUAUUAAUCUUCACC | |
| AD-2011362 | asusguu(Gda)UfuUfAfUfguuucuuacuL96 | | asGfsuaaGfaAfAfcauaAfaCfaacausgsa | | UCAUGUUGUUUAUGUUUCUUACC | |
| AD-2011363 | asusacu(Cda)GfgAfCfUfcaaaugucuuL96 | | asAfsgacAfuUfUfgaguCfcGfaguausasu | | AUAUACUCGGACUCAAAUGUCUC | |
| AD-2011364 | usgsaag(Cda)UfuUfUfCfugauaauuauL96 | | asUfsaauUfaUfCfagaaAfaGfcuucasasc | | GUUGAAGCUUUUCUGAUAAUUAU | |
| AD-2011365 | gsasacu(Cda)UfuUfGfCfuguuaaucuuL96 | | asAfsgauUfaAfCfagcaAfaGfaguucsusa | | UAGAACUCUUUGCUGUUAAUCUG | |
| AD-2011366 | ususgaa(Ada)GfuUfUfUfgcugauuaauL96 | | asUfsuaaUfcAfGfcaaaAfcUfuucaasusg | | CAUUGAAAGUUUUGCUGAUUAAC | |
| AD-2011367 | asusgcu(Ada)GfaAfGfAfaaacuaaaguL96 | | asCfsuuuAfgUfUfuucuUfcUfagcaususa | | UAAUGCUAGAAGAAAACUAAAGG | |
| AD-2011368 | cscsucu(Uda)CfaUfGfCfauguuucuguL96 | | asCfsagaAfaCfAfugcaUfgAfagaggsasg | | CUCCUCUUCAUGCAUGUUUCUGA | |
| AD-2011369 | usascug(Cda)UfuUfUfGfaaccugaaguL96 | | asCfsuucAfgGfUfucaaAfaGfcaguasasg | | CUUACUGCUUUUGAACCUGAAGC | |
| AD-2011370 | csasacg(Uda)UfcUfAfCfuguugcuucuL96 | | asGfsaagCfaAfCfaguaGfaAfcguugsasc | | GUCAACGUUCUACUGUUGCUUCC | |
| AD-2011371 | csasgau(Ada)GfuCfCfUfuguuuuaauuL96 | | asAfsuuaAfaAfCfaaggAfcUfaucugsgsg | | CCCAGAUAGUCCUUGUUUUAAUG | |
| AD-2011372 | ascsacu(Gda)UfaUfAfUfauuucuugcuL96 | | asGfscaaGfaAfAfuauaUfaCfaguguscsc | | GGACACUGUAUAUAUUUCUUGCC | |
| AD-2011373 | asascuu(Ada)AfuAfAfAfgauauuguguL96 | | asCfsacaAfuAfUfcuuuAfuUfaaguusgsg | | CCAACUUAAUAAAGAUAUUGUGA | |
| AD-2011374 | ususucu(Ada)UfuUfGfUfgucuccucuuL96 | | asAfsgagGfaGfAfcacaAfaUfagaaasusa | | UAUUUCUAUUUGUGUCUCCUCUA | |
| AD-2011375 | gscsuua(Cda)AfaAfUfGfuuugccauuuL96 | | asAfsaugGfcAfAfacauUfuGfuaagcscsu | | AGGCUUACAAAUGUUUGCCAUUC | |
| AD-2011376 | csusgug(Ada)UfuUfUfUfguucucuucuL96 | | asGfsaagAfgAfAfcaaaAfaUfcacagsgsa | | UCCUGUGAUUUUUGUUCUCUUCC | |
| AD-2011377 | usgsauc(Gda)AfaGfCfUfaucaacaauuL96 | | asAfsuugUfuGfAfuagcUfuCfgaucasgsu | | ACUGAUCGAAGCUAUCAACAAUG | |
| AD-2011378 | asusgcc(Uda)GfuUfUfAfugcuguucauL96 | | asUfsgaaCfaGfCfauaaAfcAfggcausgsu | | ACAUGCCUGUUUAUGCUGUUCAU | |
| AD-2011379 | asgsaau(Uda)UfcCfCfAfguuuaaaacuL96 | | asGfsuuuUfaAfAfcuggGfaAfauucusasa | | UUAGAAUUUCCCAGUUUAAAACA | |
| AD-2011380 | gsusaga(Uda)CfaAfGfUfuugucuuccuL96 | | asGfsgaaGfaCfAfaacuUfgAfucuacsasg | | CUGUAGAUCAAGUUUGUCUUCCG | |
| AD-2011381 | ascsaac(Cda)CfuUfCfCfucuuuucucuL96 | | asGfsagaAfaAfGfaggaAfgGfguugususu | | AAACAACCCUUCCUCUUUUCUCU | |
| AD-2011382 | asgsuga(Ada)AfuUfUfGfcauaaugaauL96 | | asUfsucaUfuAfUfgcaaAfuUfucacususc | | GAAGUGAAAUUUGCAUAAUGAAU | |
| AD-2011383 | ususagg(Ada)UfaUfAfUfucacauuguuL96 | | asAfscaaUfgUfGfaauaUfaUfccuaasasg | | CUUUAGGAUAUAUUCACAUUGUU | |
| AD-2011384 | uscsuua(Gda)AfuAfUfUfauugcuaguuL96 | | asAfscuaGfcAfAfuaauAfuCfuaagasusg | | CAUCUUAGAUAUUAUUGCUAGUG | |
| AD-2011385 | csusggu(Uda)AfaAfAfUfggaugauuuuL96 | | asAfsaauCfaUfCfcauuUfuAfaccagsgsa | | UCCUGGUUAAAAUGGAUGAUUUU | |
| AD-2011386 | csusucc(Cda)UfgAfAfGfugcuuuacauL96 | | asUfsguaAfaGfCfacuuCfaGfggaagsasg | | CUCUUCCCUGAAGUGCUUUACAG | |
| AD-2011387 | csascca(Cda)UfgUfCfUfugaugcucuuL96 | | asAfsgagCfaUfCfaagaCfaGfuggugsasg | | CUCACCACUGUCUUGAUGCUCUG | |
| AD-2011388 | ascsaac(Uda)CfuUfGfUfuuugcuguuuL96 | | asAfsacaGfcAfAfaacaAfgAfguugusasu | | AUACAACUCUUGUUUUGCUGUUG | |
| AD-2011389 | cscsuuu(Cda)GfuUfUfGfcuuucuuauuL96 | | asAfsuaaGfaAfAfgcaaAfcGfaaaggsasa | | UUCCUUUCGUUUGCUUUCUUAUU | |
| AD-2011390 | gsgsgca(Ada)AfaUfCfAfcuuaugaaauL96 | | asUfsuucAfuAfAfgugaUfuUfugcccsasc | | GUGGGCAAAAUCACUUAUGAAAG | |
| AD-2011391 | asascac(Uda)UfuGfAfUfguuaucauuuL96 | | asAfsaugAfuAfAfcaucAfaAfguguusasc | | GUAACACUUUGAUGUUAUCAUUU | |
| AD-2011392 | ususccu(Cda)CfaAfGfGfaguuagaauuL96 | | asAfsuucUfaAfCfuccuUfgGfaggaasgsa | | UCUUCCUCCAAGGAGUUAGAAUU | |
| AD-2011393 | asasgug(Uda)UfuUfGfAfacuugaucuuL96 | | asAfsgauCfaAfGfuucaAfaAfcacuusasu | | AUAAGUGUUUUGAACUUGAUCUU | |
| AD-2011394 | usgsgcc(Uda)CfaUfUfUfuucucuuuuuL96 | | asAfsaaaGfaGfAfaaaaUfgAfggccasusu | | AAUGGCCUCAUUUUUCUCUUUUU | |
| AD-2011395 | asgsgua(Uda)UfuGfCfAfuuguuuaaauL96 | | asUfsuuaAfaCfAfaugcAfaAfuaccuscsc | | GGAGGUAUUUGCAUUGUUUAAAG | |
| AD-2011396 | cscsuaa(Uda)AfuUfGfUfgcgacuucauL96 | | asUfsgaaGfuCfGfcacaAfuAfuuaggsgsu | | ACCCUAAUAUUGUGCGACUUCAU | |
| AD-2011397 | ascsacc(Ada)AfaUfGfAfaguggucauuL96 | | asAfsugaCfcAfCfuucaUfuUfgguguscsu | | AGACACCAAAUGAAGUGGUCAUC | |
| AD-2011398 | gsusagc(Ada)AfaUfCfAfguauauucuuL96 | | asAfsgaaUfaUfAfcugaUfuUfgcuacsusa | | UAGUAGCAAAUCAGUAUAUUCUA | |
| AD-2011399 | csgsaaa(Gda)CfaAfGfAfgauuaucaauL96 | | asUfsugaUfaAfUfcucuUfgCfuuucgsusg | | CACGAAAGCAAGAGAUUAUCAAA | |
| AD-2011400 | cscsaac(Ada)CfaGfAfCfucuaucagcuL96 | | asGfscugAfuAfGfagucUfgUfguuggsusc | | GACCAACACAGACUCUAUCAGCA | |
| AD-2011401 | gsascua(Gda)AfaUfGfAfuauuugauauL96 | | asUfsaucAfaAfUfaucaUfuCfuagucsasc | | GUGACUAGAAUGAUAUUUGAUAG | |
| AD-2011402 | gscscug(Ada)GfaAfUfUfugcuuuuaguL96 | | asCfsuaaAfaGfCfaaauUfcUfcaggcsusu | | AAGCCUGAGAAUUUGCUUUUAGC | |
| AD-2011403 | csasaaa(Cda)AfaUfGfGfucucuucuguL96 | | asCfsagaAfgAfGfaccaUfuGfuuuugscsu | | AGCAAAACAAUGGUCUCUUCUGG | |
| AD-2011404 | gsasuaa(Ada)CfaAfCfAfaagccaacguL96 | | asCfsguuGfgCfUfuuguUfgUfuuaucsusu | | AAGAUAAACAACAAAGCCAACGU | |
| AD-2011405 | ususuuu(Uda)AfaUfAfCfgaaccugucuL96 | | asGfsacaGfgUfUfcguaUfuAfaaaaasasa | | UUUUUUUUAAUACGAACCUGUCC | |
| AD-2011406 | gsgsuuu(Ada)UfaAfUfUfcugcuuaaguL96 | | asCfsuuaAfgCfAfgaauUfaUfaaaccsusu | | AAGGUUUAUAAUUCUGCUUAAGC | |
| AD-2011407 | asgsgau(Ada)AfaAfGfCfaaguuccucuL96 | | asGfsaggAfaCfUfugcuUfuUfauccusasa | | UUAGGAUAAAAGCAAGUUCCUCC | |
| AD-2011408 | usgsuag(Ada)GfuGfUfUfuuuuuuacauL96 | | asUfsguaAfaAfAfaaacAfcUfcuacasusc | | GAUGUAGAGUGUUUUUUUUACAC | |
| AD-2011409 | ususcuc(Uda)GfaAfUfGfacaguuguauL96 | | asUfsacaAfcUfGfucauUfcAfgagaascsa | | UGUUCUCUGAAUGACAGUUGUAA | |
| AD-2011410 | asusugu(Ada)UfuUfAfCfcucuccuguuL96 | | asAfscagGfaGfAfgguaAfaUfacaauscsu | | AGAUUGUAUUUACCUCUCCUGUC | |
| AD-2011411 | usgsgca(Uda)AfcAfUfGfuauuuaaaguL96 | | asCfsuuuAfaAfUfacauGfuAfugccascsg | | CGUGGCAUACAUGUAUUUAAAGA | |
| AD-2011412 | ususguc(Ada)AfgAfUfUfuuaaagguuuL96 | | asAfsaccUfuUfAfaaauCfuUfgacaascsa | | UGUUGUCAAGAUUUUAAAGGUUU | |
| AD-2011413 | usasuga(Ada)AfaUfUfCfcuacuggacuL96 | | asGfsuccAfgUfAfggaaUfuUfucauascsa | | UGUAUGAAAAUUCCUACUGGACA | |
| AD-2011414 | cscsucu(Uda)UfgUfGfGfcaaaacaucuL96 | | asGfsaugUfuUfUfgccaCfaAfagaggsusg | | CACCUCUUUGUGGCAAAACAUCU | |
| AD-2011415 | csasguc(Ada)GfaAfGfAfgacucguguuL96 | | asAfscacGfaGfUfcucuUfcUfgacugscsa | | UGCAGUCAGAAGAGACUCGUGUG | |
| AD-2011416 | asasguc(Ada)CfuGfAfAfcaacugaucuL96 | | asGfsaucAfgUfUfguucAfgUfgacuususg | | CAAAGUCACUGAACAACUGAUCG | |
| AD-2011417 | gsgsagc(Uda)UfaUfGfAfuuuuccaucuL96 | | asGfsaugGfaAfAfaucaUfaAfgcuccsasg | | CUGGAGCUUAUGAUUUUCCAUCA | |
| AD-2011418 | gsusgcg(Gda)AfuCfGfUfuucgcaacuuL96 | | asAfsguuGfcGfAfaacgAfuCfcgcacsusg | | CAGUGCGGAUCGUUUCGCAACUG | |
| AD-2011419 | asasucu(Gda)CfcGfUfCfuuuugaagcuL96 | | asGfscuuCfaAfAfagacGfgCfagauuscsu | | AGAAUCUGCCGUCUUUUGAAGCA | |
| AD-2011420 | usascuu(Cda)UfuUfUfCfccgacuucuuL96 | | asAfsgaaGfuCfGfggaaAfaGfaaguasgsg | | CCUACUUCUUUUCCCGACUUCUU | |
| AD-2011421 | csusacu(Gda)UfaAfUfCfcacaacccuuL96 | | asAfsgggUfuGfUfggauUfaCfaguagsusu | | AACUACUGUAAUCCACAACCCUG | |
| AD-2011422 | ususugu(Uda)CfcAfUfUfcuuuucuuauL96 | | asUfsaagAfaAfAfgaauGfgAfacaaasasg | | CUUUUGUUCCAUUCUUUUCUUAC | |
| AD-2011423 | gsasaga(Uda)GfuGfAfAfagcacgaaauL96 | | asUfsuucGfuGfCfuuucAfcAfucuucsasu | | AUGAAGAUGUGAAAGCACGAAAG | |
| AD-2011424 | csasgua(Ada)AfuAfUfAfuugagccauuL96 | | asAfsuggCfuCfAfauauAfuUfuacugsusa | | UACAGUAAAUAUAUUGAGCCAUG | |
| AD-2011425 | asasugg(Gda)AfaAfGfAfaaacuucucuL96 | | asGfsagaAfgUfUfuucuUfuCfccauususg | | CAAAUGGGAAAGAAAACUUCUCA | |
| AD-2011426 | gsgscag(Ada)AfuGfUfUfcauuuucauuL96 | | asAfsugaAfaAfUfgaacAfuUfcugccsasc | | GUGGCAGAAUGUUCAUUUUCAUC | |
| AD-2011427 | csgsccu(Gda)CfaUfAfGfcauauauuauL96 | | asUfsaauAfuAfUfgcuaUfgCfaggcgsgsc | | GCCGCCUGCAUAGCAUAUAUUAG | |
| AD-2011428 | csusggc(Uda)GfuUfUfUfuccauuuccuL96 | | asGfsgaaAfuGfGfaaaaAfcAfgccagsgsc | | GCCUGGCUGUUUUUCCAUUUCCC | |
| AD-2011429 | asgsgga(Ada)AfuAfAfAfagucuuuuguL96 | | asCfsaaaAfgAfCfuuuuAfuUfucccususu | | AAAGGGAAAUAAAAGUCUUUUGA | |
| AD-2011430 | cscsacc(Cda)UfuUfCfUfggucaucucuL96 | | asGfsagaUfgAfCfcagaAfaGfgguggscsg | | CGCCACCCUUUCUGGUCAUCUCC | |
| AD-2011431 | usgscca(Uda)AfaUfGfGfuaaaggacuuL96 | | asAfsgucCfuUfUfaccaUfuAfuggcasasg | | CUUGCCAUAAUGGUAAAGGACUG | |
| AD-2011432 | csasaac(Uda)GfuGfUfAfaacuggaaauL96 | | asUfsuucCfaGfUfuuacAfcAfguuugsasc | | GUCAAACUGUGUAAACUGGAAAA | |
| AD-2011433 | usasaac(Cda)CfuCfAfUfguacaucuguL96 | | asCfsagaUfgUfAfcaugAfgGfguuuasgsa | | UCUAAACCCUCAUGUACAUCUGG | |
| AD-2011434 | ususuug(Uda)GfaAfGfCfagcuauacguL96 | | asCfsguaUfaGfCfugcuUfcAfcaaaasgsg | | CCUUUUGUGAAGCAGCUAUACGU | |
| AD-2011435 | asusgga(Gda)AfuAfUfCfauugauaaauL96 | | asUfsuuaUfcAfAfugauAfuCfuccaususc | | GAAUGGAGAUAUCAUUGAUAAAU | |
| AD-2011436 | gsusgua(Uda)UfcAfUfCfauugcauucuL96 | | asGfsaauGfcAfAfugauGfaAfuacacsasg | | CUGUGUAUUCAUCAUUGCAUUCC | |
| AD-2011437 | gsasagc(Uda)UfuGfGfGfuaauuuaguuL96 | | asAfscuaAfaUfUfacccAfaAfgcuucsasg | | CUGAAGCUUUGGGUAAUUUAGUG | |
| AD-2011438 | gscsugu(Gda)AfaAfUfUfggcagacuuuL96 | | asAfsaguCfuGfCfcaauUfuCfacagcsusg | | CAGCUGUGAAAUUGGCAGACUUU | |
| AD-2011439 | usasaca(Ada)UfuAfAfAfgugggaugauL96 | | asUfscauCfcCfAfcuuuAfaUfuguuasasu | | AUUAACAAUUAAAGUGGGAUGAU | |
| AD-2011440 | gscsugg(Cda)UfaCfAfAfggaauuucuuL96 | | asAfsgaaAfuUfCfcuugUfaGfccagcsasu | | AUGCUGGCUACAAGGAAUUUCUC | |
| AD-2011441 | usgsuua(Uda)UfaUfUfCfuuugcucacuL96 | | asGfsugaGfcAfAfagaaUfaAfuaacasasu | | AUUGUUAUUAUUCUUUGCUCACG | |
| AD-2011442 | asusaac(Ada)AfuUfUfGfcacuugguguL96 | | asCfsaccAfaGfUfgcaaAfuUfguuausgsu | | ACAUAACAAUUUGCACUUGGUGA | |
| AD-2011443 | csusucc(Gda)CfaCfUfAfagaugugaguL96 | | asCfsucaCfaUfCfuuagUfgCfggaagsasc | | GUCUUCCGCACUAAGAUGUGAGG | |
| AD-2011444 | csuscuc(Ada)UfcUfCfAfccucucuguuL96 | | asAfscagAfgAfGfgugaGfaUfgagagsasa | | UUCUCUCAUCUCACCUCUCUGUG | |
| AD-2011445 | csasccc(Ada)UfgGfAfUfcugucaacguL96 | | asCfsguuGfaCfAfgaucCfaUfgggugscsu | | AGCACCCAUGGAUCUGUCAACGU | |
| AD-2011446 | asusaaa(Ada)UfuCfUfAfcugacuucuuL96 | | asAfsgaaGfuCfAfguagAfaUfuuuaususc | | GAAUAAAAUUCUACUGACUUCUA | |
| AD-2011447 | usgscca(Uda)CfuUfGfAfcaacuaugcuL96 | | asGfscauAfgUfUfgucaAfgAfuggcascsc | | GGUGCCAUCUUGACAACUAUGCU | |
| AD-2011448 | ascsuua(Gda)GfuAfUfCfcuaacuauguL96 | | asCfsauaGfuUfAfggauAfcCfuaaguscsc | | GGACUUAGGUAUCCUAACUAUGU | |
| AD-2011449 | csusgcu(Uda)CfuAfCfUfccuccugcuuL96 | | asAfsgcaGfgAfGfgaguAfgAfagcagsasg | | CUCUGCUUCUACUCCUCCUGCUC | |
| AD-2011450 | usgsagg(Ada)AfaUfUfGfugauuuguuuL96 | | asAfsacaAfaUfCfacaaUfuUfccucascsa | | UGUGAGGAAAUUGUGAUUUGUUC | |
| AD-2011451 | gsascga(Gda)UfaUfCfAfgcuuuucgauL96 | | asUfscgaAfaAfGfcugaUfaCfucgucscsg | | CGGACGAGUAUCAGCUUUUCGAG | |
| AD-2011452 | uscsccu(Cda)UfgAfCfCfcucaguuucuL96 | | asGfsaaaCfuGfAfggguCfaGfagggasasg | | CUUCCCUCUGACCCUCAGUUUCC | |
| AD-2011453 | gscsccu(Uda)UfuGfAfUfaacagaagcuL96 | | asGfscuuCfuGfUfuaucAfaAfagggcsusu | | AAGCCCUUUUGAUAACAGAAGCC | |
| AD-2011454 | asgsaaa(Uda)UfaAfAfUfgguagcaguuL96 | | asAfscugCfuAfCfcauuUfaAfuuucusasu | | AUAGAAAUUAAAUGGUAGCAGUA | |
| AD-2011455 | gsasugg(Ada)UfuUfUfCfaccgauucuuL96 | | asAfsgaaUfcGfGfugaaAfaUfccaucscsc | | GGGAUGGAUUUUCACCGAUUCUA | |
| AD-2011456 | ascsauc(Ada)CfaCfUfUfgcucacauguL96 | | asCfsaugUfgAfGfcaagUfgUfgaugususu | | AAACAUCACACUUGCUCACAUGC | |
| AD-2011457 | uscsuaa(Gda)GfcCfUfGfaaaaccauuuL96 | | asAfsaugGfuUfUfucagGfcCfuuagasusg | | CAUCUAAGGCCUGAAAACCAUUC | |
| AD-2011458 | ususacg(Uda)AfaAfGfAfuccuuaugguL96 | | asCfscauAfaGfGfaucuUfuAfcguaasasa | | UUUUACGUAAAGAUCCUUAUGGA | |
| AD-2011459 | usascag(Uda)GfaAfGfCfugaugccaguL96 | | asCfsuggCfaUfCfagcuUfcAfcuguasgsu | | ACUACAGUGAAGCUGAUGCCAGU | |
| AD-2011460 | asusuug(Cda)UfgAfAfUfugaauuguuuL96 | | asAfsacaAfuUfCfaauuCfaGfcaaaususc | | GAAUUUGCUGAAUUGAAUUGUUU | |
| AD-2011461 | usasagc(Uda)CfuAfGfUfuuggacuuauL96 | | asUfsaagUfcCfAfaacuAfgAfgcuuasasg | | CUUAAGCUCUAGUUUGGACUUAG | |
| AD-2011462 | csusggc(Uda)AfgUfAfGfugugugagauL96 | | asUfscucAfcAfCfacuaCfuAfgccagscsc | | GGCUGGCUAGUAGUGUGUGAGAA | |
| AD-2011463 | asusgug(Ada)UfgCfAfUfcaucuuaucuL96 | | asGfsauaAfgAfUfgaugCfaUfcacausasu | | AUAUGUGAUGCAUCAUCUUAUCA | |
| AD-2011464 | cscsuac(Ada)UfgUfAfAfugcauauguuL96 | | asAfscauAfuGfCfauuaCfaUfguaggsasc | | GUCCUACAUGUAAUGCAUAUGUG | |
| AD-2011465 | csasgca(Gda)CfcAfAfGfaguuuguuguL96 | | asCfsaacAfaAfCfucuuGfgCfugcugsasg | | CUCAGCAGCCAAGAGUUUGUUGA | |
| AD-2011466 | uscscaa(Uda)CfcAfGfCfcuucacauguL96 | | asCfsaugUfgAfAfggcuGfgAfuuggasgsg | | CCUCCAAUCCAGCCUUCACAUGG | |
| AD-2011467 | ascscaa(Cda)AfgUfAfCfccaucaagcuL96 | | asGfscuuGfaUfGfgguaCfuGfuuggusgsa | | UCACCAACAGUACCCAUCAAGCC | |
| AD-2011468 | csusgcu(Ada)GfgGfAfUfcaucagaaauL96 | | asUfsuucUfgAfUfgaucCfcUfagcagsasa | | UUCUGCUAGGGAUCAUCAGAAAC | |
| AD-2011469 | gsgsuuu(Gda)CfuAfCfCfacauaaagcuL96 | | asGfscuuUfaUfGfugguAfgCfaaaccsasa | | UUGGUUUGCUACCACAUAAAGCC | |
| AD-2011470 | asgsugu(Ada)CfaGfCfAfucaugcuccuL96 | | asGfsgagCfaUfGfaugcUfgUfacacusgsa | | UCAGUGUACAGCAUCAUGCUCCA | |
| AD-2011471 | usgsgcu(Uda)CfgAfCfCfacaaccugcuL96 | | asGfscagGfuUfGfugguCfgAfagccasusc | | GAUGGCUUCGACCACAACCUGCA | |
| AD-2011472 | cscsagu(Cda)AfuUfGfCfauucagcaguL96 | | asCfsugcUfgAfAfugcaAfuGfacuggscsa | | UGCCAGUCAUUGCAUUCAGCAGA | |
| AD-2011473 | csusucc(Ada)UfgAfUfGfcacagacaguL96 | | asCfsuguCfuGfUfgcauCfaUfggaagscsa | | UGCUUCCAUGAUGCACAGACAGG | |
| AD-2011474 | csasgga(Gda)GfaUfAfCfcaacuugauuL96 | | asAfsucaAfgUfUfgguaUfcCfuccugsgsc | | GCCAGGAGGAUACCAACUUGAUA | |
| AD-2011475 | usgsaug(Gda)AfaAfCfAfaggagucaauL96 | | asUfsugaCfuCfCfuuguUfuCfcaucasgsg | | CCUGAUGGAAACAAGGAGUCAAC | |
| AD-2011476 | asasgca(Ada)GfaAfUfCfaguugguuuuL96 | | asAfsaacCfaAfCfugauUfcUfugcuuscsu | | AGAAGCAAGAAUCAGUUGGUUUG | |
| AD-2011477 | csasacu(Gda)CfuUfGfCfcacucguccuL96 | | asGfsgacGfaGfUfggcaAfgCfaguugscsg | | CGCAACUGCUUGCCACUCGUCCC | |
| AD-2011478 | gsusguu(Gda)AfaGfUfAfuuacuguaguL96 | | asCfsuacAfgUfAfauacUfuCfaacacscsa | | UGGUGUUGAAGUAUUACUGUAGA | |
| AD-2011479 | ususacu(Gda)UfuGfGfCfaaaacaauauL96 | | asUfsauuGfuUfUfugccAfaCfaguaasusu | | AAUUACUGUUGGCAAAACAAUAG | |
| AD-2011480 | csasgug(Uda)GfuCfCfUfacucuggucuL96 | | asGfsaccAfgAfGfuaggAfcAfcacugscsc | | GGCAGUGUGUCCUACUCUGGUCU | |
| AD-2011481 | asgsaaa(Cda)CfaGfAfUfggaguaaaguL96 | | asCfsuuuAfcUfCfcaucUfgGfuuucususc | | GAAGAAACCAGAUGGAGUAAAGA | |
| AD-2011482 | gscsucc(Ada)CfaGfCfAfaaccuuccuuL96 | | asAfsggaAfgGfUfuugcUfgUfggagcsasu | | AUGCUCCACAGCAAACCUUCCUA | |
| AD-2011483 | gsascuu(Uda)GfaAfGfCfcuacacaaauL96 | | asUfsuugUfgUfAfggcuUfcAfaagucscsc | | GGGACUUUGAAGCCUACACAAAA | |

TABLE 43
Unmodified Sense and Antisense Strand Sequences of CAMK2D dsRNA Agents Comprising a GalNAc Conjugate
Targeting Ligand

| | | SEQ | Range in | | | SEQ | Range in | |
|---|---|---|---|---|---|---|---|---|
| Duplex | Sense Strand Sequence | ID | NM_001321571.2 | | Antisense Strand Sequence | ID | NM_001321571.2 | |
| Name | 5′ to 3′ | NO. | Start | End | 5′ to 3′ | NO. | Start | End |
| AD-2005899 | GUGCGGAUCGUUUCGCAACUU | | 278 | 298 | AAGUUGCGAAACGAUCCGCACUG | | 276 | 298 |
| AD-2005914 | CAACUGCUUGCCACUCGUCCU | | 293 | 313 | AGGACGAGUGGCAAGCAGUUGCG | | 291 | 313 |
| AD-2005989 | CUGGCUGUUUUUCCAUUUCCU | | 318 | 338 | AGGAAAUGGAAAAACAGCCAGGC | | 316 | 338 |
| AD-2006091 | CCACCCUUUCUGGUCAUCUCU | | 481 | 501 | AGAGAUGACCAGAAAGGGUGGCG | | 479 | 501 |
| AD-2006275 | UGGCUUCGACCACAACCUGCU | | 672 | 692 | AGCAGGUUGUGGUCGAAGCCAUC | | 670 | 692 |
| AD-2006307 | GACGAGUAUCAGCUUUUCGAU | | 704 | 724 | AUCGAAAAGCUGAUACUCGUCCG | | 702 | 724 |
| AD-2006395 | UAUGAAAAUUCCUACUGGACU | | 763 | 783 | AGUCCAGUAGGAAUUUUCAUACA | | 761 | 783 |
| AD-2006473 | GCUGCCAAAAUUAUCAACACU | | 791 | 811 | AGUGUUGAUAAUUUUGGCAGCAU | | 789 | 811 |
| AD-2006501 | CUGCUAGGGAUCAUCAGAAAU | | 822 | 842 | AUUUCUGAUGAUCCCUAGCAGAA | | 820 | 842 |
| AD-2006588 | AAUCUGCCGUCUUUUGAAGCU | | 859 | 879 | AGCUUCAAAAGACGGCAGAUUCU | | 857 | 879 |
| AD-2006610 | CCUAAUAUUGUGCGACUUCAU | | 881 | 901 | AUGAAGUCGCACAAUAUUAGGGU | | 879 | 901 |
| AD-2006710 | UUGGUGUUUGAUUUAGUUACU | | 932 | 952 | AGUAACUAAAUCAAACACCAAGU | | 930 | 952 |
| AD-2006870 | UACAGUGAAGCUGAUGCCAGU | | 992 | 1012 | ACUGGCAUCAGCUUCACUGUAGU | | 990 | 1012 |
| AD-2006886 | CCAGUCAUUGCAUUCAGCAGU | | 1008 | 1028 | ACUGCUGAAUGCAAUGACUGGCA | | 1006 | 1028 |
| AD-2007012 | GCCUGAGAAUUUGCUUUUAGU | | 1084 | 1104 | ACUAAAAGCAAAUUCUCAGGCUU | | 1082 | 1104 |
| AD-2007079 | GCUGUGAAAUUGGCAGACUUU | | 1124 | 1144 | AAAGUCUGCCAAUUUCACAGCUG | | 1122 | 1144 |
| AD-2007144 | UCUUUCUCCAGAAGUUUUACU | | 1210 | 1230 | AGUAAAACUUCUGGAGAAAGAUA | | 1208 | 1230 |
| AD-2007160 | UUACGUAAAGAUCCUUAUGGU | | 1226 | 1246 | ACCAUAAGGAUCUUUACGUAAAA | | 1224 | 1246 |
| AD-2007211 | AUUCUCUAUAUUCUACUUGUU | | 1277 | 1297 | AACAAGUAGAAUAUAGAGAAUGA | | 1275 | 1297 |
| AD-2007238 | CCAACACAGACUCUAUCAGCU | | 1324 | 1344 | AGCUGAUAGAGUCUGUGUUGGUC | | 1322 | 1344 |
| AD-2007269 | GGAGCUUAUGAUUUUCCAUCU | | 1355 | 1375 | AGAUGGAAAAUCAUAAGCUCCAG | | 1353 | 1375 |
| AD-2007325 | CCUCAUCAAUAAAAUGCUUAU | | 1411 | 1431 | AUAAGCAUUUUAUUGAUGAGGUC | | 1409 | 1431 |
| AD-2007389 | CACCCAUGGAUCUGUCAACGU | | 1475 | 1495 | ACGUUGACAGAUCCAUGGGUGCU | | 1473 | 1495 |
| AD-2007404 | CAACGUUCUACUGUUGCUUCU | | 1490 | 1510 | AGAAGCAACAGUAGAACGUUGAC | | 1488 | 1510 |
| AD-2007420 | CUUCCAUGAUGCACAGACAGU | | 1506 | 1526 | ACUGUCUGUGCAUCAUGGAAGCA | | 1504 | 1526 |
| AD-2007451 | CUGCUUGAAGAAAUUUAAUGU | | 1537 | 1557 | ACAUUAAAUUUCUUCAAGCAGUC | | 1535 | 1557 |
| AD-2007468 | AUGCUAGAAGAAAACUAAAGU | | 1554 | 1574 | ACUUUAGUUUUCUUCUAGCAUUA | | 1552 | 1574 |
| AD-2007490 | UGCCAUCUUGACAACUAUGCU | | 1576 | 1596 | AGCAUAGUUGUCAAGAUGGCACC | | 1574 | 1596 |
| AD-2007508 | GCUGGCUACAAGGAAUUUCUU | | 1594 | 1614 | AAGAAAUUCCUUGUAGCCAGCAU | | 1592 | 1614 |
| AD-2007528 | CAGCAGCCAAGAGUUUGUUGU | | 1614 | 1634 | ACAACAAACUCUUGGCUGCUGAG | | 1612 | 1634 |
| AD-2007549 | AGAAACCAGAUGGAGUAAAGU | | 1635 | 1655 | ACUUUACUCCAUCUGGUUUCUUC | | 1633 | 1655 |
| AD-2007568 | GAUAAACAACAAAGCCAACGU | | 1654 | 1674 | ACGUUGGCUUUGUUGUUUAUCUU | | 1652 | 1674 |
| AD-2007585 | CUACUGUAAUCCACAACCCUU | | 1725 | 1745 | AAGGGUUGUGGAUUACAGUAGUU | | 1723 | 1745 |
| AD-2007604 | UGAUGGAAACAAGGAGUCAAU | | 1744 | 1764 | AUUGACUCCUUGUUUCCAUCAGG | | 1742 | 1764 |
| AD-2007627 | AGAGUUCAAAUACAACAAUUU | | 1767 | 1787 | AAAUUGUUGUAUUUGAACUCUCA | | 1765 | 1787 |
| AD-2007653 | GAAGAUGUGAAAGCACGAAAU | | 1793 | 1813 | AUUUCGUGCUUUCACAUCUUCAU | | 1791 | 1813 |
| AD-2007668 | CGAAAGCAAGAGAUUAUCAAU | | 1808 | 1828 | AUUGAUAAUCUCUUGCUUUCGUG | | 1806 | 1828 |
| AD-2007687 | AAGUCACUGAACAACUGAUCU | | 1827 | 1847 | AGAUCAGUUGUUCAGUGACUUUG | | 1825 | 1847 |
| AD-2007702 | UGAUCGAAGCUAUCAACAAUU | | 1842 | 1862 | AAUUGUUGAUAGCUUCGAUCAGU | | 1840 | 1862 |
| AD-2007705 | GACUUUGAAGCCUACACAAAU | | 1865 | 1885 | AUUUGUGUAGGCUUCAAAGUCCC | | 1863 | 1885 |
| AD-2007743 | UACUGCUUUUGAACCUGAAGU | | 1903 | 1923 | ACUUCAGGUUCAAAAGCAGUAAG | | 1901 | 1923 |
| AD-2007759 | GAAGCUUUGGGUAAUUUAGUU | | 1919 | 1939 | AACUAAAUUACCCAAAGCUUCAG | | 1917 | 1939 |
| AD-2007785 | GAUGGAUUUUCACCGAUUCUU | | 1945 | 1965 | AAGAAUCGGUGAAAAUCCAUCCC | | 1943 | 1965 |
| AD-2007801 | UUCUACUUUGAAAAUGCUUUU | | 1961 | 1981 | AAAAGCAUUUUCAAAGUAGAAUC | | 1959 | 1981 |
| AD-2007818 | UUUGUCCAAAAGCAAUAAACU | | 1978 | 1998 | AGUUUAUUGCUUUUGGACAAAGC | | 1976 | 1998 |
| AD-2007841 | UCCACACUAUUAUUCUAAACU | | 2001 | 2021 | AGUUUAGAAUAAUAGUGUGGAUU | | 1999 | 2021 |
| AD-2007856 | UAAACCCUCAUGUACAUCUGU | | 2016 | 2036 | ACAGAUGUACAUGAGGGUUUAGA | | 2014 | 2036 |
| AD-2007870 | CGCCUGCAUAGCAUAUAUUAU | | 2050 | 2070 | AUAAUAUAUGCUAUGCAGGCGGC | | 2048 | 2070 |
| AD-2007934 | CAGUCAGAAGAGACUCGUGUU | | 2114 | 2134 | AACACGAGUCUCUUCUGACUGCA | | 2112 | 2134 |
| AD-2007974 | GGCAGAAUGUUCAUUUUCAUU | | 2157 | 2177 | AAUGAAAAUGAACAUUCUGCCAC | | 2155 | 2177 |
| AD-2007985 | ACCAACAGUACCCAUCAAGCU | | 2188 | 2208 | AGCUUGAUGGGUACUGUUGGUGA | | 2186 | 2208 |
| AD-2008019 | AAUGGGAAAGAAAACUUCUCU | | 2222 | 2242 | AGAGAAGUUUUCUUUCCCAUUUG | | 2220 | 2242 |
| AD-2008047 | CCUCUUUGUGGCAAAACAUCU | | 2250 | 2270 | AGAUGUUUUGCCACAAAGAGGUG | | 2248 | 2270 |
| AD-2008065 | UCUAAGGCCUGAAAACCAUUU | | 2268 | 2288 | AAAUGGUUUUCAGGCCUUAGAUG | | 2266 | 2288 |
| AD-2008093 | GGUCUUCUAAAUUUCAACAGU | | 2296 | 2316 | ACUGUUGAAAUUUAGAAGACCCA | | 2294 | 2316 |
| AD-2008116 | CACUUCUGCAUUCUCUGUUCU | | 2319 | 2339 | AGAACAGAGAAUGCAGAAGUGGC | | 2317 | 2339 |
| AD-2008170 | CCUCUUCAUGCAUGUUUCUGU | | 2378 | 2398 | ACAGAAACAUGCAUGAAGAGGAG | | 2376 | 2398 |
| AD-2008211 | CCUACAUGUAAUGCAUAUGUU | | 2419 | 2439 | AACAUAUGCAUUACAUGUAGGAC | | 2417 | 2439 |
| AD-2008227 | AUGUGAUGCAUCAUCUUAUCU | | 2435 | 2455 | AGAUAAGAUGAUGCAUCACAUAU | | 2433 | 2455 |
| AD-2008242 | UAUCAUAUAUUCCUUCCUAUU | | 2451 | 2471 | AAUAGGAAGGAAUAUAUGAUAAG | | 2449 | 2471 |
| AD-2008257 | CCUAUACAUUGUUUACACUUU | | 2466 | 2486 | AAAGUGUAAACAAUGUAUAGGAA | | 2464 | 2486 |
| AD-2008273 | CCACACAAACUUAAAUUACUU | | 2502 | 2522 | AAGUAAUUUAAGUUUGUGUGGAA | | 2500 | 2522 |
| AD-2008288 | UUACUGUUGGCAAAACAAUAU | | 2517 | 2537 | AUAUUGUUUUGCCAACAGUAAUU | | 2515 | 2537 |
| AD-2008310 | CCAAGUACAACUCUUCAUCAU | | 2573 | 2593 | AUGAUGAAGAGUUGUACUUGGAA | | 2571 | 2593 |
| AD-2008325 | CAUCAAGUUUCUCUGUUAAUU | | 2588 | 2608 | AAUUAACAGAGAAACUUGAUGAA | | 2586 | 2608 |
| AD-2008357 | CAGACUUAAGAACUAUUGUUU | | 2620 | 2640 | AAACAAUAGUUCUUAAGUCUGUU | | 2618 | 2640 |
| AD-2008375 | UUCUCUGAAUGACAGUUGUAU | | 2638 | 2658 | AUACAACUGUCAUUCAGAGAACA | | 2636 | 2658 |
| AD-2008402 | GAACUCUUUGCUGUUAAUCUU | | 2678 | 2698 | AAGAUUAACAGCAAAGAGUUCUA | | 2676 | 2698 |
| AD-2008453 | CUGGUUAAAAUGGAUGAUUUU | | 2772 | 2792 | AAAAUCAUCCAUUUUAACCAGGA | | 2770 | 2792 |
| AD-2008474 | UUGAAAGUUUUGCUGAUUAAU | | 2796 | 2816 | AUUAAUCAGCAAAACUUUCAAUG | | 2794 | 2816 |
| AD-2008491 | UAACAAUUAAAGUGGGAUGAU | | 2813 | 2833 | AUCAUCCCACUUUAAUUGUUAAU | | 2811 | 2833 |
| AD-2008516 | GGGCAAAAUCACUUAUGAAAU | | 2838 | 2858 | AUUUCAUAAGUGAUUUUGCCCAC | | 2836 | 2858 |
| AD-2008540 | AAGCAAGAAUCAGUUGGUUUU | | 2862 | 2882 | AAAACCAACUGAUUCUUGCUUCU | | 2860 | 2882 |
| AD-2008555 | GGUUUGCUACCACAUAAAGCU | | 2877 | 2897 | AGCUUUAUGUGGUAGCAAACCAA | | 2875 | 2897 |
| AD-2008575 | CAUGCUGUUUUUGGUCAAACU | | 2897 | 2917 | AGUUUGACCAAAAACAGCAUGGC | | 2895 | 2917 |
| AD-2008590 | CAAACUGUGUAAACUGGAAAU | | 2912 | 2932 | AUUUCCAGUUUACACAGUUUGAC | | 2910 | 2932 |
| AD-2008623 | UCUGAGUUUAAUCACUUUAGU | | 2945 | 2965 | ACUAAAGUGAUUAAACUCAGAAA | | 2943 | 2965 |
| AD-2008639 | UUAGGAUAUAUUCACAUUGUU | | 2961 | 2981 | AACAAUGUGAAUAUAUCCUAAAG | | 2959 | 2981 |
| AD-2008666 | AUUUGCUGAAUUGAAUUGUUU | | 2989 | 3009 | AAACAAUUCAAUUCAGCAAAUUC | | 2987 | 3009 |
| AD-2008687 | UUUCUCAAAUCUGUGAUCUCU | | 3013 | 3033 | AGAGAUCACAGAUUUGAGAAAGA | | 3011 | 3033 |
| AD-2008701 | AUCUCUUUUCUUUAUCCUGUU | | 3028 | 3048 | AACAGGAUAAAGAAAAGAGAUCA | | 3026 | 3048 |
| AD-2008708 | CCUUUCGUUUGCUUUCUUAUU | | 3057 | 3077 | AAUAAGAAAGCAAACGAAAGGAA | | 3055 | 3077 |
| AD-2008714 | UUUGUUCCAUUCUUUUCUUAU | | 3083 | 3103 | AUAAGAAAAGAAUGGAACAAAAG | | 3081 | 3103 |
| AD-2008720 | CUGGCUAGUAGUGUGUGAGAU | | 3132 | 3152 | AUCUCACACACUACUAGCCAGCC | | 3130 | 3152 |
| AD-2008746 | AGUGAAAUUUGCAUAAUGAAU | | 3162 | 3182 | AUUCAUUAUGCAAAUUUCACUUC | | 3160 | 3182 |
| AD-2008767 | AGGGAAAUAAAAGUCUUUUGU | | 3188 | 3208 | ACAAAAGACUUUUAUUUCCCUUU | | 3186 | 3208 |
| AD-2008799 | UAGCACUUUUGAUCAUCUUCU | | 3221 | 3241 | AGAAGAUGAUCAAAAGUGCUAGU | | 3219 | 3241 |
| AD-2008835 | UUGUCAAGAUUUUAAAGGUUU | | 3257 | 3277 | AAACCUUUAAAAUCUUGACAACA | | 3255 | 3277 |
| AD-2008844 | GGUUUAUAAUUCUGCUUAAGU | | 3273 | 3293 | ACUUAAGCAGAAUUAUAAACCUU | | 3271 | 3293 |
| AD-2008860 | UAAGCUCUAGUUUGGACUUAU | | 3289 | 3309 | AUAAGUCCAAACUAGAGCUUAAG | | 3287 | 3309 |
| AD-2008875 | ACUUAGGUAUCCUAACUAUGU | | 3304 | 3324 | ACAUAGUUAGGAUACCUAAGUCC | | 3302 | 3324 |
| AD-2008899 | AGGUAUUUGCAUUGUUUAAAU | | 3328 | 3348 | AUUUAAACAAUGCAAAUACCUCC | | 3326 | 3348 |
| AD-2008921 | AGGAUAAAAGCAAGUUCCUCU | | 3351 | 3371 | AGAGGAACUUGCUUUUAUCCUAA | | 3349 | 3371 |
| AD-2008941 | CUGUGACUGCAACGUCUUACU | | 3371 | 3391 | AGUAAGACGUUGCAGUCACAGGA | | 3369 | 3391 |
| AD-2008977 | CAGGAGGAUACCAACUUGAUU | | 3407 | 3427 | AAUCAAGUUGGUAUCCUCCUGGC | | 3405 | 3427 |
| AD-2009021 | UCAUCACUAGCUAGUUUUCUU | | 3492 | 3512 | AAGAAAACUAGCUAGUGAUGAUG | | 3490 | 3512 |
| AD-2009053 | GCUUACAAAUGUUUGCCAUUU | | 3524 | 3544 | AAAUGGCAAACAUUUGUAAGCCU | | 3522 | 3544 |
| AD-2009078 | AAGUGUUUUGAACUUGAUCUU | | 3549 | 3569 | AAGAUCAAGUUCAAAACACUUAU | | 3547 | 3569 |
| AD-2009108 | UGCUUUUUUAGCUUCUCUCUU | | 3579 | 3599 | AAGAGAGAAGCUAAAAAAGCACA | | 3577 | 3599 |
| AD-2009122 | UCUCUUGAAUCAGAGUAUCAU | | 3594 | 3614 | AUGAUACUCUGAUUCAAGAGAGA | | 3592 | 3614 |
| AD-2009147 | UUCCUCCAAGGAGUUAGAAUU | | 3619 | 3639 | AAUUCUAACUCCUUGGAGGAAGA | | 3617 | 3639 |
| AD-2009162 | AGAAUUUCCCAGUUUAAAACU | | 3634 | 3654 | AGUUUUAAACUGGGAAAUUCUAA | | 3632 | 3654 |
| AD-2009197 | GGUUUUCUUUGUGCUUCUCAU | | 3672 | 3692 | AUGAGAAGCACAAAGAAAACCUA | | 3670 | 3692 |
| AD-2009211 | CUCAUUUUUCCUUUGUUGAUU | | 3688 | 3708 | AAUCAACAAAGGAAAAAUGAGAA | | 3686 | 3708 |
| AD-2009238 | CUGUGAUUUUUGUUCUCUUCU | | 3715 | 3735 | AGAAGAGAACAAAAAUCACAGGA | | 3713 | 3735 |
| AD-2009253 | CUUCCCUGAAGUGCUUUACAU | | 3731 | 3751 | AUGUAAAGCACUUCAGGGAAGAG | | 3729 | 3751 |
| AD-2009285 | UCCAUCAUUGUUAUUUUAACU | | 3763 | 3783 | AGUUAAAAUAACAAUGAUGGAGA | | 3761 | 3783 |
| AD-2009302 | AUAGUAAUUCACAGUCCUCAU | | 3784 | 3804 | AUGAGGACUGUGAAUUACUAUCG | | 3782 | 3804 |
| AD-2009317 | CCUCAGAAGCCUAUUUUUAAU | | 3799 | 3819 | AUUAAAAAUAGGCUUCUGAGGAC | | 3797 | 3819 |
| AD-2009344 | ACAACCCUUCCUCUUUUCUCU | | 3853 | 3873 | AGAGAAAAGAGGAAGGGUUGUUU | | 3851 | 3873 |
| AD-2009354 | CUCUCAUCUCACCUCUCUGUU | | 3870 | 3890 | AACAGAGAGGUGAGAUGAGAGAA | | 3868 | 3890 |
| AD-2009369 | UCUGUGUUGAUUACUAAUCAU | | 3885 | 3905 | AUGAUUAGUAAUCAACACAGAGA | | 3883 | 3905 |
| AD-2009386 | UCUUAGAUAUUAUUGCUAGUU | | 3905 | 3925 | AACUAGCAAUAAUAUCUAAGAUG | | 3903 | 3925 |
| AD-2009425 | UGAAGCUUUUCUGAUAAUUAU | | 3944 | 3964 | AUAAUUAUCAGAAAAGCUUCAAC | | 3942 | 3964 |
| AD-2009437 | CAGUAAAUAUAUUGAGCCAUU | | 4007 | 4027 | AAUGGCUCAAUAUAUUUACUGUA | | 4005 | 4027 |
| AD-2009490 | UGGCCUCAUUUUUCUCUUUUU | | 4060 | 4080 | AAAAAGAGAAAAAUGAGGCCAUU | | 4058 | 4080 |
| AD-2009505 | UUUUGUGAAGCAGCUAUACGU | | 4095 | 4115 | ACGUAUAGCUGCUUCACAAAAGG | | 4093 | 4115 |
| AD-2009525 | UGGCAUACAUGUAUUUAAAGU | | 4115 | 4135 | ACUUUAAAUACAUGUAUGCCACG | | 4113 | 4135 |
| AD-2009537 | UGUAGAGUGUUUUUUUUACAU | | 4148 | 4168 | AUGUAAAAAAAACACUCUACAUC | | 4146 | 4168 |
| AD-2009546 | UACACUUUUAACUUAGCAUGU | | 4164 | 4184 | ACAUGCUAAGUUAAAAGUGUAAA | | 4162 | 4184 |
| AD-2009568 | GUGUUGAAGUAUUACUGUAGU | | 4186 | 4206 | ACUACAGUAAUACUUCAACACCA | | 4184 | 4206 |
| AD-2009584 | GUAGAUCAAGUUUGUCUUCCU | | 4202 | 4222 | AGGAAGACAAACUUGAUCUACAG | | 4200 | 4222 |
| AD-2009599 | CUUCCGCACUAAGAUGUGAGU | | 4217 | 4237 | ACUCACAUCUUAGUGCGGAAGAC | | 4215 | 4237 |
| AD-2009615 | UGAGGAAAUUGUGAUUUGUUU | | 4233 | 4253 | AAACAAAUCACAAUUUCCUCACA | | 4231 | 4253 |
| AD-2009643 | CACAAAUGAAUUACACAUUUU | | 4261 | 4281 | AAAAUGUGUAAUUCAUUUGUGGU | | 4259 | 4281 |
| AD-2009655 | UUCUAUCAUUUUGAAACACUU | | 4287 | 4307 | AAGUGUUUCAAAAUGAUAGAAGA | | 4285 | 4307 |
| AD-2009690 | ACACUGUAUAUAUUUCUUGCU | | 4322 | 4342 | AGCAAGAAAUAUAUACAGUGUCC | | 4320 | 4342 |
| AD-2009707 | UGCCAUAAUGGUAAAGGACUU | | 4339 | 4359 | AAGUCCUUUACCAUUAUGGCAAG | | 4337 | 4359 |
| AD-2009722 | GACUGAUUGAUAUAUUUAAGU | | 4355 | 4375 | ACUUAAAUAUAUCAAUCAGUCCU | | 4353 | 4375 |
| AD-2009755 | CGUCCAUCUUUAUUUCUUCAU | | 4407 | 4427 | AUGAAGAAAUAAAGAUGGACGCA | | 4405 | 4427 |
| AD-2009823 | CAGUGUGUCCUACUCUGGUCU | | 4475 | 4495 | AGACCAGAGUAGGACACACUGCC | | 4473 | 4495 |
| AD-2009839 | GGUCUCAUUUAGUACAUAACU | | 4491 | 4511 | AGUUAUGUACUAAAUGAGACCAG | | 4489 | 4511 |
| AD-2009852 | AUAACAAUUUGCACUUGGUGU | | 4506 | 4526 | ACACCAAGUGCAAAUUGUUAUGU | | 4504 | 4526 |
| AD-2009881 | AGUUAAUUGUUCUCUGUGAGU | | 4535 | 4555 | ACUCACAGAGAACAAUUAACUUG | | 4533 | 4555 |
| AD-2009901 | CAAAACAAUGGUCUCUUCUGU | | 4555 | 4575 | ACAGAAGAGACCAUUGUUUUGCU | | 4553 | 4575 |
| AD-2009931 | GCUGAGAACAAUAUAGUUAAU | | 4585 | 4605 | AUUAACUAUAUUGUUCUCAGCAA | | 4583 | 4605 |
| AD-2009959 | CUCCUAAAAGCUUCUCUAAAU | | 4614 | 4634 | AUUUAGAGAAGCUUUUAGGAGUC | | 4612 | 4634 |
| AD-2009987 | UCCAAUCCAGCCUUCACAUGU | | 4642 | 4662 | ACAUGUGAAGGCUGGAUUGGAGG | | 4640 | 4662 |
| AD-2010007 | UUUUUUAAUACGAACCUGUCU | | 4678 | 4698 | AGACAGGUUCGUAUUAAAAAAAA | | 4676 | 4698 |
| AD-2010032 | AACACUUUGAUGUUAUCAUUU | | 4703 | 4723 | AAAUGAUAACAUCAAAGUGUUAC | | 4701 | 4723 |
| AD-2010054 | CAGCUUGAACUUGAGCAUACU | | 4762 | 4782 | AGUAUGCUCAAGUUCAAGCUGGG | | 4760 | 4782 |
| AD-2010084 | AGCUUCUUUUGAUUUGCUAAU | | 4792 | 4812 | AUUAGCAAAUCAAAAGAAGCUGA | | 4790 | 4812 |
| AD-2010106 | ACAUCACACUUGCUCACAUGU | | 4814 | 4834 | ACAUGUGAGCAAGUGUGAUGUUU | | 4812 | 4834 |
| AD-2010123 | AUGCCUGUUUAUGCUGUUCAU | | 4831 | 4851 | AUGAACAGCAUAAACAGGCAUGU | | 4829 | 4851 |
| AD-2010142 | AUGUUGUUUAUGUUUCUUACU | | 4850 | 4870 | AGUAAGAAACAUAAACAACAUGA | | 4848 | 4870 |
| AD-2010158 | UUACCUAGAAUAAAUAGUCUU | | 4866 | 4886 | AAGACUAUUUAUUCUAGGUAAGA | | 4864 | 4886 |
| AD-2010165 | UACUUCUUUUCCCGACUUCUU | | 4893 | 4913 | AAGAAGUCGGGAAAAGAAGUAGG | | 4891 | 4913 |
| AD-2010184 | UUACUUUUUCCUAAGAUUCAU | | 4912 | 4932 | AUGAAUCUUAGGAAAAAGUAAGA | | 4910 | 4932 |
| AD-2010203 | AGUGUACAGCAUCAUGCUCCU | | 4931 | 4951 | AGGAGCAUGAUGCUGUACACUGA | | 4929 | 4951 |
| AD-2010218 | GCUCCACAGCAAACCUUCCUU | | 4946 | 4966 | AAGGAAGGUUUGCUGUGGAGCAU | | 4944 | 4966 |
| AD-2010262 | CCUCUCAAAACCUACAUAAUU | | 4990 | 5010 | AAUUAUGUAGGUUUUGAGAGGGA | | 4988 | 5010 |
| AD-2010280 | AUUGUAUUUACCUCUCCUGUU | | 5012 | 5032 | AACAGGAGAGGUAAAUACAAUCU | | 5010 | 5032 |
| AD-2010301 | AACCACAUUGUUUUGAAAAUU | | 5033 | 5053 | AAUUUUCAAAACAAUGUGGUUGA | | 5031 | 5053 |
| AD-2010309 | UUUCUAUUUGUGUCUCCUCUU | | 5056 | 5076 | AAGAGGAGACACAAAUAGAAAUA | | 5054 | 5076 |
| AD-2010325 | CUCUACUGCAGUAUAAUGUCU | | 5072 | 5092 | AGACAUUAUACUGCAGUAGAGGA | | 5070 | 5092 |
| AD-2010361 | GUGUAUUCAUCAUUGCAUUCU | | 5108 | 5128 | AGAAUGCAAUGAUGAAUACACAG | | 5106 | 5128 |
| AD-2010404 | AUGGAGAUAUCAUUGAUAAAU | | 5151 | 5171 | AUUUAUCAAUGAUAUCUCCAUUC | | 5149 | 5171 |
| AD-2010441 | GCCCUUUUGAUAACAGAAGCU | | 5192 | 5212 | AGCUUCUGUUAUCAAAAGGGCUU | | 5190 | 5212 |
| AD-2010454 | CAGAUAGUCCUUGUUUUAAUU | | 5225 | 5245 | AAUUAAAACAAGGACUAUCUGGG | | 5223 | 5245 |
| AD-2010486 | CACCACUGUCUUGAUGCUCUU | | 5257 | 5277 | AAGAGCAUCAAGACAGUGGUGAG | | 5255 | 5277 |
| AD-2010503 | UCUGAGCAAGUUACCUCUUCU | | 5274 | 5294 | AGAAGAGGUAACUUGCUCAGAGC | | 5272 | 5294 |
| AD-2010521 | UCCCUCUGACCCUCAGUUUCU | | 5292 | 5312 | AGAAACUGAGGGUCAGAGGGAAG | | 5290 | 5312 |
| AD-2010535 | GUUUCCAUAUUUGUAAAAUGU | | 5307 | 5327 | ACAUUUUACAAAUAUGGAAACUG | | 5305 | 5327 |
| AD-2010545 | AUGAGAAUAAACAUACCAACU | | 5324 | 5344 | AGUUGGUAUGUUUAUUCUCAUUU | | 5322 | 5344 |
| AD-2010559 | AACUUAAUAAAGAUAUUGUGU | | 5341 | 5361 | ACACAAUAUCUUUAUUAAGUUGG | | 5339 | 5361 |
| AD-2010598 | GACUAGAAUGAUAUUUGAUAU | | 5381 | 5401 | AUAUCAAAUAUCAUUCUAGUCAC | | 5379 | 5401 |
| AD-2010609 | AGAAAUUAAAUGGUAGCAGUU | | 5400 | 5420 | AACUGCUACCAUUUAAUUUCUAU | | 5398 | 5420 |
| AD-2010624 | GCAGUAUAACUAUUCUGAUCU | | 5415 | 5435 | AGAUCAGAAUAGUUAUACUGCUA | | 5413 | 5435 |
| AD-2010643 | CACUGACAUUAAUAUUCCUAU | | 5434 | 5454 | AUAGGAAUAUUAAUGUCAGUGAU | | 5432 | 5454 |
| AD-2010655 | UGUUAUUAUUCUUUGCUCACU | | 5455 | 5475 | AGUGAGCAAAGAAUAAUAACAAU | | 5453 | 5475 |
| AD-2010683 | ACAACUCUUGUUUUGCUGUUU | | 5483 | 5503 | AAACAGCAAAACAAGAGUUGUAU | | 5481 | 5503 |
| AD-2010708 | GCCCUCUUUAUGUAGGUUUAU | | 5508 | 5528 | AUAAACCUACAUAAAGAGGGCAG | | 5506 | 5528 |
| AD-2010745 | AUACUCGGACUCAAAUGUCUU | | 5545 | 5565 | AAGACAUUUGAGUCCGAGUAUAU | | 5543 | 5565 |
| AD-2010782 | ACACCAAAUGAAGUGGUCAUU | | 5582 | 5602 | AAUGACCACUUCAUUUGGUGUCU | | 5580 | 5602 |
| AD-2010830 | GUAGCAAAUCAGUAUAUUCUU | | 5631 | 5651 | AAGAAUAUACUGAUUUGCUACUA | | 5629 | 5651 |
| AD-2010855 | CAAGGAAUAUUAAUCUUCACU | | 5662 | 5682 | AGUGAAGAUUAAUAUUCCUUGAA | | 5660 | 5682 |
| AD-2010871 | UCACCCAAAUUUUGAAUUUUU | | 5678 | 5698 | AAAAAUUCAAAAUUUGGGUGAAG | | 5676 | 5698 |
| AD-2010911 | GAGUGAUUUCAUUGAAUAAAU | | 5746 | 5766 | AUUUAUUCAAUGAAAUCACUCGA | | 5744 | 5766 |
| AD-2010919 | AUAAAAUUCUACUGACUUCUU | | 5761 | 5781 | AAGAAGUCAGUAGAAUUUUAUUC | | 5759 | 5781 |

TABLE 44
Modified Sense and Antisense Strand Sequences of CAMK2D dsRNA Agents Comprising a GalNAc Conjugate Targeting Ligand

| | | SEQ | | SEQ | | SEQ |
|---|---|---|---|---|---|---|
| Duplex | | ID | | ID | | ID |
| Name | Sense Strand Sequence 5′ to 3′ | NO. | Antisense Strand Sequence 5′ to 3′ | NO. | mRNA target sequence | NO. |
| AD-2005876 | csusgcuuCfuAfCfUfccuccugcuuL96 | | asAfsgcaGfgAfGfgaguAfgAfagcagsasg | | CUCUGCUUCUACUCCUCCUGCUC | |
| AD-2005899 | gsusgcggAfuCfGfUfuucgcaacuuL96 | | asAfsguuGfcGfAfaacgAfuCfcgcacsusg | | CAGUGCGGAUCGUUUCGCAACUG | |
| AD-2005914 | csasacugCfuUfGfCfcacucguccuL96 | | asGfsgacGfaGfUfggcaAfgCfaguugscsg | | CGCAACUGCUUGCCACUCGUCCC | |
| AD-2005989 | csusggcuGfuUfUfUfuccauuuccuL96 | | asGfsgaaAfuGfGfaaaaAfcAfgccagsgsc | | GCCUGGCUGUUUUUCCAUUUCCC | |
| AD-2006091 | cscsacccUfuUfCfUfggucaucucuL96 | | asGfsagaUfgAfCfcagaAfaGfgguggscsg | | CGCCACCCUUUCUGGUCAUCUCC | |
| AD-2006275 | usgsgcuuCfgAfCfCfacaaccugcuL96 | | asGfscagGfuUfGfugguCfgAfagccasusc | | GAUGGCUUCGACCACAACCUGCA | |
| AD-2006307 | gsascgagUfaUfCfAfgcuuuucgauL96 | | asUfscgaAfaAfGfcugaUfaCfucgucscsg | | CGGACGAGUAUCAGCUUUUCGAG | |
| AD-2006395 | usasugaaAfaUfUfCfcuacuggacuL96 | | asGfsuccAfgUfAfggaaUfuUfucauascsa | | UGUAUGAAAAUUCCUACUGGACA | |
| AD-2006473 | gscsugccAfaAfAfUfuaucaacacuL96 | | asGfsuguUfgAfUfaauuUfuGfgcagcsasu | | AUGCUGCCAAAAUUAUCAACACC | |
| AD-2006501 | csusgcuaGfgGfAfUfcaucagaaauL96 | | asUfsuucUfgAfUfgaucCfcUfagcagsasa | | UUCUGCUAGGGAUCAUCAGAAAC | |
| AD-2006588 | asasucugCfcGfUfCfuuuugaagcuL96 | | asGfscuuCfaAfAfagacGfgCfagauuscsu | | AGAAUCUGCCGUCUUUUGAAGCA | |
| AD-2006610 | cscsuaauAfuUfGfUfgcgacuucauL96 | | asUfsgaaGfuCfGfcacaAfuAfuuaggsgsu | | ACCCUAAUAUUGUGCGACUUCAU | |
| AD-2006710 | ususggugUfuUfGfAfuuuaguuacuL96 | | asGfsuaaCfuAfAfaucaAfaCfaccaasgsu | | ACUUGGUGUUUGAUUUAGUUACU | |
| AD-2006870 | usascaguGfaAfGfCfugaugccaguL96 | | asCfsuggCfaUfCfagcuUfcAfcuguasgsu | | ACUACAGUGAAGCUGAUGCCAGU | |
| AD-2006886 | cscsagucAfuUfGfCfauucagcaguL96 | | asCfsugcUfgAfAfugcaAfuGfacuggscsa | | UGCCAGUCAUUGCAUUCAGCAGA | |
| AD-2007012 | gscscugaGfaAfUfUfugcuuuuaguL96 | | asCfsuaaAfaGfCfaaauUfcUfcaggcsusu | | AAGCCUGAGAAUUUGCUUUUAGC | |
| AD-2007079 | gscsugugAfaAfUfUfggcagacuuuL96 | | asAfsaguCfuGfCfcaauUfuCfacagcsusg | | CAGCUGUGAAAUUGGCAGACUUU | |
| AD-2007144 | uscsuuucUfcCfAfGfaaguuuuacuL96 | | asGfsuaaAfaCfUfucugGfaGfaaagasusa | | UAUCUUUCUCCAGAAGUUUUACG | |
| AD-2007160 | ususacguAfaAfGfAfuccuuaugguL96 | | asCfscauAfaGfGfaucuUfuAfcguaasasa | | UUUUACGUAAAGAUCCUUAUGGA | |
| AD-2007211 | asusucucUfaUfAfUfucuacuuguuL96 | | asAfscaaGfuAfGfaauaUfaGfagaausgsa | | UCAUUCUCUAUAUUCUACUUGUG | |
| AD-2007238 | cscsaacaCfaGfAfCfucuaucagcuL96 | | asGfscugAfuAfGfagucUfgUfguuggsusc | | GACCAACACAGACUCUAUCAGCA | |
| AD-2007269 | gsgsagcuUfaUfGfAfuuuuccaucuL96 | | asGfsaugGfaAfAfaucaUfaAfgcuccsasg | | CUGGAGCUUAUGAUUUUCCAUCA | |
| AD-2007325 | cscsucauCfaAfUfAfaaaugcuuauL96 | | asUfsaagCfaUfUfuuauUfgAfugaggsusc | | GACCUCAUCAAUAAAAUGCUUAC | |
| AD-2007389 | csascccaUfgGfAfUfcugucaacguL96 | | asCfsguuGfaCfAfgaucCfaUfgggugscsu | | AGCACCCAUGGAUCUGUCAACGU | |
| AD-2007404 | csasacguUfcUfAfCfuguugcuucuL96 | | asGfsaagCfaAfCfaguaGfaAfcguugsasc | | GUCAACGUUCUACUGUUGCUUCC | |
| AD-2007420 | csusuccaUfgAfUfGfcacagacaguL96 | | asCfsuguCfuGfUfgcauCfaUfggaagscsa | | UGCUUCCAUGAUGCACAGACAGG | |
| AD-2007451 | csusgcuuGfaAfGfAfaauuuaauguL96 | | asCfsauuAfaAfUfuucuUfcAfagcagsusc | | GACUGCUUGAAGAAAUUUAAUGC | |
| AD-2007468 | asusgcuaGfaAfGfAfaaacuaaaguL96 | | asCfsuuuAfgUfUfuucuUfcUfagcaususa | | UAAUGCUAGAAGAAAACUAAAGG | |
| AD-2007490 | usgsccauCfuUfGfAfcaacuaugcuL96 | | asGfscauAfgUfUfgucaAfgAfuggcascsc | | GGUGCCAUCUUGACAACUAUGCU | |
| AD-2007508 | gscsuggcUfaCfAfAfggaauuucuuL96 | | asAfsgaaAfuUfCfcuugUfaGfccagcsasu | | AUGCUGGCUACAAGGAAUUUCUC | |
| AD-2007528 | csasgcagCfcAfAfGfaguuuguuguL96 | | asCfsaacAfaAfCfucuuGfgCfugcugsasg | | CUCAGCAGCCAAGAGUUUGUUGA | |
| AD-2007549 | asgsaaacCfaGfAfUfggaguaaaguL96 | | asCfsuuuAfcUfCfcaucUfgGfuuucususc | | GAAGAAACCAGAUGGAGUAAAGA | |
| AD-2007568 | gsasuaaaCfaAfCfAfaagccaacguL96 | | asCfsguuGfgCfUfuuguUfgUfuuaucsusu | | AAGAUAAACAACAAAGCCAACGU | |
| AD-2007585 | csusacugUfaAfUfCfcacaacccuuL96 | | asAfsgggUfuGfUfggauUfaCfaguagsusu | | AACUACUGUAAUCCACAACCCUG | |
| AD-2007604 | usgsauggAfaAfCfAfaggagucaauL96 | | asUfsugaCfuCfCfuuguUfuCfcaucasgsg | | CCUGAUGGAAACAAGGAGUCAAC | |
| AD-2007627 | asgsaguuCfaAfAfUfacaacaauuuL96 | | asAfsauuGfuUfGfuauuUfgAfacucuscsa | | UGAGAGUUCAAAUACAACAAUUG | |
| AD-2007653 | gsasagauGfuGfAfAfagcacgaaauL96 | | asUfsuucGfuGfCfuuucAfcAfucuucsasu | | AUGAAGAUGUGAAAGCACGAAAG | |
| AD-2007668 | csgsaaagCfaAfGfAfgauuaucaauL96 | | asUfsugaUfaAfUfcucuUfgCfuuucgsusg | | CACGAAAGCAAGAGAUUAUCAAA | |
| AD-2007687 | asasgucaCfuGfAfAfcaacugaucuL96 | | asGfsaucAfgUfUfguucAfgUfgacuususg | | CAAAGUCACUGAACAACUGAUCG | |
| AD-2007702 | usgsaucgAfaGfCfUfaucaacaauuL96 | | asAfsuugUfuGfAfuagcUfuCfgaucasgsu | | ACUGAUCGAAGCUAUCAACAAUG | |
| AD-2007705 | gsascuuuGfaAfGfCfcuacacaaauL96 | | asUfsuugUfgUfAfggcuUfcAfaagucscsc | | GGGACUUUGAAGCCUACACAAAA | |
| AD-2007743 | usascugcUfuUfUfGfaaccugaaguL96 | | asCfsuucAfgGfUfucaaAfaGfcaguasasg | | CUUACUGCUUUUGAACCUGAAGC | |
| AD-2007759 | gsasagcuUfuGfGfGfuaauuuaguuL96 | | asAfscuaAfaUfUfacccAfaAfgcuucsasg | | CUGAAGCUUUGGGUAAUUUAGUG | |
| AD-2007785 | gsasuggaUfuUfUfCfaccgauucuuL96 | | asAfsgaaUfcGfGfugaaAfaUfccaucscsc | | GGGAUGGAUUUUCACCGAUUCUA | |
| AD-2007801 | ususcuacUfuUfGfAfaaaugcuuuuL96 | | asAfsaagCfaUfUfuucaAfaGfuagaasusc | | GAUUCUACUUUGAAAAUGCUUUG | |
| AD-2007818 | ususugucCfaAfAfAfgcaauaaacuL96 | | asGfsuuuAfuUfGfcuuuUfgGfacaaasgsc | | GCUUUGUCCAAAAGCAAUAAACC | |
| AD-2007841 | uscscacaCfuAfUfUfauucuaaacuL96 | | asGfsuuuAfgAfAfuaauAfgUfguggasusu | | AAUCCACACUAUUAUUCUAAACC | |
| AD-2007856 | usasaaccCfuCfAfUfguacaucuguL96 | | asCfsagaUfgUfAfcaugAfgGfguuuasgsa | | UCUAAACCCUCAUGUACAUCUGG | |
| AD-2007870 | csgsccugCfaUfAfGfcauauauuauL96 | | asUfsaauAfuAfUfgcuaUfgCfaggcgsgsc | | GCCGCCUGCAUAGCAUAUAUUAG | |
| AD-2007934 | csasgucaGfaAfGfAfgacucguguuL96 | | asAfscacGfaGfUfcucuUfcUfgacugscsa | | UGCAGUCAGAAGAGACUCGUGUG | |
| AD-2007974 | gsgscagaAfuGfUfUfcauuuucauuL96 | | asAfsugaAfaAfUfgaacAfuUfcugccsasc | | GUGGCAGAAUGUUCAUUUUCAUC | |
| AD-2007985 | ascscaacAfgUfAfCfccaucaagcuL96 | | asGfscuuGfaUfGfgguaCfuGfuuggusgsa | | UCACCAACAGUACCCAUCAAGCC | |
| AD-2008019 | asasugggAfaAfGfAfaaacuucucuL96 | | asGfsagaAfgUfUfuucuUfuCfccauususg | | CAAAUGGGAAAGAAAACUUCUCA | |
| AD-2008047 | cscsucuuUfgUfGfGfcaaaacaucuL96 | | asGfsaugUfuUfUfgccaCfaAfagaggsusg | | CACCUCUUUGUGGCAAAACAUCU | |
| AD-2008065 | uscsuaagGfcCfUfGfaaaaccauuuL96 | | asAfsaugGfuUfUfucagGfcCfuuagasusg | | CAUCUAAGGCCUGAAAACCAUUC | |
| AD-2008093 | gsgsucuuCfuAfAfAfuuucaacaguL96 | | asCfsuguUfgAfAfauuuAfgAfagaccscsa | | UGGGUCUUCUAAAUUUCAACAGU | |
| AD-2008116 | csascuucUfgCfAfUfucucuguucuL96 | | asGfsaacAfgAfGfaaugCfaGfaagugsgsc | | GCCACUUCUGCAUUCUCUGUUCU | |
| AD-2008170 | cscsucuuCfaUfGfCfauguuucuguL96 | | asCfsagaAfaCfAfugcaUfgAfagaggsasg | | CUCCUCUUCAUGCAUGUUUCUGA | |
| AD-2008211 | cscsuacaUfgUfAfAfugcauauguuL96 | | asAfscauAfuGfCfauuaCfaUfguaggsasc | | GUCCUACAUGUAAUGCAUAUGUG | |
| AD-2008227 | asusgugaUfgCfAfUfcaucuuaucuL96 | | asGfsauaAfgAfUfgaugCfaUfcacausasu | | AUAUGUGAUGCAUCAUCUUAUCA | |
| AD-2008242 | usasucauAfuAfUfUfccuuccuauuL96 | | asAfsuagGfaAfGfgaauAfuAfugauasasg | | CUUAUCAUAUAUUCCUUCCUAUA | |
| AD-2008257 | cscsuauaCfaUfUfGfuuuacacuuuL96 | | asAfsaguGfuAfAfacaaUfgUfauaggsasa | | UUCCUAUACAUUGUUUACACUUC | |
| AD-2008273 | cscsacacAfaAfCfUfuaaauuacuuL96 | | asAfsguaAfuUfUfaaguUfuGfuguggsasa | | UUCCACACAAACUUAAAUUACUG | |
| AD-2008288 | ususacugUfuGfGfCfaaaacaauauL96 | | asUfsauuGfuUfUfugccAfaCfaguaasusu | | AAUUACUGUUGGCAAAACAAUAG | |
| AD-2008310 | cscsaaguAfcAfAfCfucuucaucauL96 | | asUfsgauGfaAfGfaguuGfuAfcuuggsasa | | UUCCAAGUACAACUCUUCAUCAA | |
| AD-2008325 | csasucaaGfuUfUfCfucuguuaauuL96 | | asAfsuuaAfcAfGfagaaAfcUfugaugsasa | | UUCAUCAAGUUUCUCUGUUAAUG | |
| AD-2008357 | csasgacuUfaAfGfAfacuauuguuuL96 | | asAfsacaAfuAfGfuucuUfaAfgucugsusu | | AACAGACUUAAGAACUAUUGUUC | |
| AD-2008375 | ususcucuGfaAfUfGfacaguuguauL96 | | asUfsacaAfcUfGfucauUfcAfgagaascsa | | UGUUCUCUGAAUGACAGUUGUAA | |
| AD-2008402 | gsasacucUfuUfGfCfuguuaaucuuL96 | | asAfsgauUfaAfCfagcaAfaGfaguucsusa | | UAGAACUCUUUGCUGUUAAUCUG | |
| AD-2008453 | csusgguuAfaAfAfUfggaugauuuuL96 | | asAfsaauCfaUfCfcauuUfuAfaccagsgsa | | UCCUGGUUAAAAUGGAUGAUUUU | |
| AD-2008474 | ususgaaaGfuUfUfUfgcugauuaauL96 | | asUfsuaaUfcAfGfcaaaAfcUfuucaasusg | | CAUUGAAAGUUUUGCUGAUUAAC | |
| AD-2008491 | usasacaaUfuAfAfAfgugggaugauL96 | | asUfscauCfcCfAfcuuuAfaUfuguuasasu | | AUUAACAAUUAAAGUGGGAUGAU | |
| AD-2008516 | gsgsgcaaAfaUfCfAfcuuaugaaauL96 | | asUfsuucAfuAfAfgugaUfuUfugcccsasc | | GUGGGCAAAAUCACUUAUGAAAG | |
| AD-2008540 | asasgcaaGfaAfUfCfaguugguuuuL96 | | asAfsaacCfaAfCfugauUfcUfugcuuscsu | | AGAAGCAAGAAUCAGUUGGUUUG | |
| AD-2008555 | gsgsuuugCfuAfCfCfacauaaagcuL96 | | asGfscuuUfaUfGfugguAfgCfaaaccsasa | | UUGGUUUGCUACCACAUAAAGCC | |
| AD-2008575 | csasugcuGfuUfUfUfuggucaaacuL96 | | asGfsuuuGfaCfCfaaaaAfcAfgcaugsgsc | | GCCAUGCUGUUUUUGGUCAAACU | |
| AD-2008590 | csasaacuGfuGfUfAfaacuggaaauL96 | | asUfsuucCfaGfUfuuacAfcAfguuugsasc | | GUCAAACUGUGUAAACUGGAAAA | |
| AD-2008623 | uscsugagUfuUfAfAfucacuuuaguL96 | | asCfsuaaAfgUfGfauuaAfaCfucagasasa | | UUUCUGAGUUUAAUCACUUUAGG | |
| AD-2008639 | ususaggaUfaUfAfUfucacauuguuL96 | | asAfscaaUfgUfGfaauaUfaUfccuaasasg | | CUUUAGGAUAUAUUCACAUUGUU | |
| AD-2008666 | asusuugcUfgAfAfUfugaauuguuuL96 | | asAfsacaAfuUfCfaauuCfaGfcaaaususc | | GAAUUUGCUGAAUUGAAUUGUUU | |
| AD-2008687 | ususucucAfaAfUfCfugugaucucuL96 | | asGfsagaUfcAfCfagauUfuGfagaaasgsa | | UCUUUCUCAAAUCUGUGAUCUCU | |
| AD-2008701 | asuscucuUfuUfCfUfuuauccuguuL96 | | asAfscagGfaUfAfaagaAfaAfgagauscsa | | UGAUCUCUUUUCUUUAUCCUGUU | |
| AD-2008708 | cscsuuucGfuUfUfGfcuuucuuauuL96 | | asAfsuaaGfaAfAfgcaaAfcGfaaaggsasa | | UUCCUUUCGUUUGCUUUCUUAUU | |
| AD-2008714 | ususuguuCfcAfUfUfcuuuucuuauL96 | | asUfsaagAfaAfAfgaauGfgAfacaaasasg | | CUUUUGUUCCAUUCUUUUCUUAC | |
| AD-2008720 | csusggcuAfgUfAfGfugugugagauL96 | | asUfscucAfcAfCfacuaCfuAfgccagscsc | | GGCUGGCUAGUAGUGUGUGAGAA | |
| AD-2008746 | asgsugaaAfuUfUfGfcauaaugaauL96 | | asUfsucaUfuAfUfgcaaAfuUfucacususc | | GAAGUGAAAUUUGCAUAAUGAAU | |
| AD-2008767 | asgsggaaAfuAfAfAfagucuuuuguL96 | | asCfsaaaAfgAfCfuuuuAfuUfucccususu | | AAAGGGAAAUAAAAGUCUUUUGA | |
| AD-2008799 | usasgcacUfuUfUfGfaucaucuucuL96 | | asGfsaagAfuGfAfucaaAfaGfugcuasgsu | | ACUAGCACUUUUGAUCAUCUUCA | |
| AD-2008835 | ususgucaAfgAfUfUfuuaaagguuuL96 | | asAfsaccUfuUfAfaaauCfuUfgacaascsa | | UGUUGUCAAGAUUUUAAAGGUUU | |
| AD-2008844 | gsgsuuuaUfaAfUfUfcugcuuaaguL96 | | asCfsuuaAfgCfAfgaauUfaUfaaaccsusu | | AAGGUUUAUAAUUCUGCUUAAGC | |
| AD-2008860 | usasagcuCfuAfGfUfuuggacuuauL96 | | asUfsaagUfcCfAfaacuAfgAfgcuuasasg | | CUUAAGCUCUAGUUUGGACUUAG | |
| AD-2008875 | ascsuuagGfuAfUfCfcuaacuauguL96 | | asCfsauaGfuUfAfggauAfcCfuaaguscsc | | GGACUUAGGUAUCCUAACUAUGU | |
| AD-2008899 | asgsguauUfuGfCfAfuuguuuaaauL96 | | asUfsuuaAfaCfAfaugcAfaAfuaccuscsc | | GGAGGUAUUUGCAUUGUUUAAAG | |
| AD-2008921 | asgsgauaAfaAfGfCfaaguuccucuL96 | | asGfsaggAfaCfUfugcuUfuUfauccusasa | | UUAGGAUAAAAGCAAGUUCCUCC | |
| AD-2008941 | csusgugaCfuGfCfAfacgucuuacuL96 | | asGfsuaaGfaCfGfuugcAfgUfcacagsgsa | | UCCUGUGACUGCAACGUCUUACU | |
| AD-2008977 | csasggagGfaUfAfCfcaacuugauuL96 | | asAfsucaAfgUfUfgguaUfcCfuccugsgsc | | GCCAGGAGGAUACCAACUUGAUA | |
| AD-2009021 | uscsaucaCfuAfGfCfuaguuuucuuL96 | | asAfsgaaAfaCfUfagcuAfgUfgaugasusg | | CAUCAUCACUAGCUAGUUUUCUA | |
| AD-2009053 | gscsuuacAfaAfUfGfuuugccauuuL96 | | asAfsaugGfcAfAfacauUfuGfuaagcscsu | | AGGCUUACAAAUGUUUGCCAUUC | |
| AD-2009078 | asasguguUfuUfGfAfacuugaucuuL96 | | asAfsgauCfaAfGfuucaAfaAfcacuusasu | | AUAAGUGUUUUGAACUUGAUCUU | |
| AD-2009108 | usgscuuuUfuUfAfGfcuucucucuuL96 | | asAfsgagAfgAfAfgcuaAfaAfaagcascsa | | UGUGCUUUUUUAGCUUCUCUCUU | |
| AD-2009122 | uscsucuuGfaAfUfCfagaguaucauL96 | | asUfsgauAfcUfCfugauUfcAfagagasgsa | | UCUCUCUUGAAUCAGAGUAUCAU | |
| AD-2009147 | ususccucCfaAfGfGfaguuagaauuL96 | | asAfsuucUfaAfCfuccuUfgGfaggaasgsa | | UCUUCCUCCAAGGAGUUAGAAUU | |
| AD-2009162 | asgsaauuUfcCfCfAfguuuaaaacuL96 | | asGfsuuuUfaAfAfcuggGfaAfauucusasa | | UUAGAAUUUCCCAGUUUAAAACA | |
| AD-2009197 | gsgsuuuuCfuUfUfGfugcuucucauL96 | | asUfsgagAfaGfCfacaaAfgAfaaaccsusa | | UAGGUUUUCUUUGUGCUUCUCAU | |
| AD-2009211 | csuscauuUfuUfCfCfuuuguugauuL96 | | asAfsucaAfcAfAfaggaAfaAfaugagsasa | | UUCUCAUUUUUCCUUUGUUGAUU | |
| AD-2009238 | csusgugaUfuUfUfUfguucucuucuL96 | | asGfsaagAfgAfAfcaaaAfaUfcacagsgsa | | UCCUGUGAUUUUUGUUCUCUUCC | |
| AD-2009253 | csusucccUfgAfAfGfugcuuuacauL96 | | asUfsguaAfaGfCfacuuCfaGfggaagsasg | | CUCUUCCCUGAAGUGCUUUACAG | |
| AD-2009285 | uscscaucAfuUfGfUfuauuuuaacuL96 | | asGfsuuaAfaAfUfaacaAfuGfauggasgsa | | UCUCCAUCAUUGUUAUUUUAACG | |
| AD-2009302 | asusaguaAfuUfCfAfcaguccucauL96 | | asUfsgagGfaCfUfgugaAfuUfacuauscsg | | CGAUAGUAAUUCACAGUCCUCAG | |
| AD-2009317 | cscsucagAfaGfCfCfuauuuuuaauL96 | | asUfsuaaAfaAfUfaggcUfuCfugaggsasc | | GUCCUCAGAAGCCUAUUUUUAAA | |
| AD-2009344 | ascsaaccCfuUfCfCfucuuuucucuL96 | | asGfsagaAfaAfGfaggaAfgGfguugususu | | AAACAACCCUUCCUCUUUUCUCU | |
| AD-2009354 | csuscucaUfcUfCfAfccucucuguuL96 | | asAfscagAfgAfGfgugaGfaUfgagagsasa | | UUCUCUCAUCUCACCUCUCUGUG | |
| AD-2009369 | uscsugugUfuGfAfUfuacuaaucauL96 | | asUfsgauUfaGfUfaaucAfaCfacagasgsa | | UCUCUGUGUUGAUUACUAAUCAU | |
| AD-2009386 | uscsuuagAfuAfUfUfauugcuaguuL96 | | asAfscuaGfcAfAfuaauAfuCfuaagasusg | | CAUCUUAGAUAUUAUUGCUAGUG | |
| AD-2009425 | usgsaagcUfuUfUfCfugauaauuauL96 | | asUfsaauUfaUfCfagaaAfaGfcuucasasc | | GUUGAAGCUUUUCUGAUAAUUAU | |
| AD-2009437 | csasguaaAfuAfUfAfuugagccauuL96 | | asAfsuggCfuCfAfauauAfuUfuacugsusa | | UACAGUAAAUAUAUUGAGCCAUG | |
| AD-2009490 | usgsgccuCfaUfUfUfuucucuuuuuL96 | | asAfsaaaGfaGfAfaaaaUfgAfggccasusu | | AAUGGCCUCAUUUUUCUCUUUUU | |
| AD-2009505 | ususuuguGfaAfGfCfagcuauacguL96 | | asCfsguaUfaGfCfugcuUfcAfcaaaasgsg | | CCUUUUGUGAAGCAGCUAUACGU | |
| AD-2009525 | usgsgcauAfcAfUfGfuauuuaaaguL96 | | asCfsuuuAfaAfUfacauGfuAfugccascsg | | CGUGGCAUACAUGUAUUUAAAGA | |
| AD-2009537 | usgsuagaGfuGfUfUfuuuuuuacauL96 | | asUfsguaAfaAfAfaaacAfcUfcuacasusc | | GAUGUAGAGUGUUUUUUUUACAC | |
| AD-2009546 | usascacuUfuUfAfAfcuuagcauguL96 | | asCfsaugCfuAfAfguuaAfaAfguguasasa | | UUUACACUUUUAACUUAGCAUGU | |
| AD-2009568 | gsusguugAfaGfUfAfuuacuguaguL96 | | asCfsuacAfgUfAfauacUfuCfaacacscsa | | UGGUGUUGAAGUAUUACUGUAGA | |
| AD-2009584 | gsusagauCfaAfGfUfuugucuuccuL96 | | asGfsgaaGfaCfAfaacuUfgAfucuacsasg | | CUGUAGAUCAAGUUUGUCUUCCG | |
| AD-2009599 | csusuccgCfaCfUfAfagaugugaguL96 | | asCfsucaCfaUfCfuuagUfgCfggaagsasc | | GUCUUCCGCACUAAGAUGUGAGG | |
| AD-2009615 | usgsaggaAfaUfUfGfugauuuguuuL96 | | asAfsacaAfaUfCfacaaUfuUfccucascsa | | UGUGAGGAAAUUGUGAUUUGUUC | |
| AD-2009643 | csascaaaUfgAfAfUfuacacauuuuL96 | | asAfsaauGfuGfUfaauuCfaUfuugugsgsu | | ACCACAAAUGAAUUACACAUUUA | |
| AD-2009655 | ususcuauCfaUfUfUfugaaacacuuL96 | | asAfsgugUfuUfCfaaaaUfgAfuagaasgsa | | UCUUCUAUCAUUUUGAAACACUG | |
| AD-2009690 | ascsacugUfaUfAfUfauuucuugcuL96 | | asGfscaaGfaAfAfuauaUfaCfaguguscsc | | GGACACUGUAUAUAUUUCUUGCC | |
| AD-2009707 | usgsccauAfaUfGfGfuaaaggacuuL96 | | asAfsgucCfuUfUfaccaUfuAfuggcasasg | | CUUGCCAUAAUGGUAAAGGACUG | |
| AD-2009722 | gsascugaUfuGfAfUfauauuuaaguL96 | | asCfsuuaAfaUfAfuaucAfaUfcagucscsu | | AGGACUGAUUGAUAUAUUUAAGA | |
| AD-2009755 | csgsuccaUfcUfUfUfauuucuucauL96 | | asUfsgaaGfaAfAfuaaaGfaUfggacgscsa | | UGCGUCCAUCUUUAUUUCUUCAG | |
| AD-2009823 | csasguguGfuCfCfUfacucuggucuL96 | | asGfsaccAfgAfGfuaggAfcAfcacugscsc | | GGCAGUGUGUCCUACUCUGGUCU | |
| AD-2009839 | gsgsucucAfuUfUfAfguacauaacuL96 | | asGfsuuaUfgUfAfcuaaAfuGfagaccsasg | | CUGGUCUCAUUUAGUACAUAACA | |
| AD-2009852 | asusaacaAfuUfUfGfcacuugguguL96 | | asCfsaccAfaGfUfgcaaAfuUfguuausgsu | | ACAUAACAAUUUGCACUUGGUGA | |
| AD-2009881 | asgsuuaaUfuGfUfUfcucugugaguL96 | | asCfsucaCfaGfAfgaacAfaUfuaacususg | | CAAGUUAAUUGUUCUCUGUGAGC | |
| AD-2009901 | csasaaacAfaUfGfGfucucuucuguL96 | | asCfsagaAfgAfGfaccaUfuGfuuuugscsu | | AGCAAAACAAUGGUCUCUUCUGG | |
| AD-2009931 | gscsugagAfaCfAfAfuauaguuaauL96 | | asUfsuaaCfuAfUfauugUfuCfucagcsasa | | UUGCUGAGAACAAUAUAGUUAAC | |
| AD-2009959 | csusccuaAfaAfGfCfuucucuaaauL96 | | asUfsuuaGfaGfAfagcuUfuUfaggagsusc | | GACUCCUAAAAGCUUCUCUAAAC | |
| AD-2009987 | uscscaauCfcAfGfCfcuucacauguL96 | | asCfsaugUfgAfAfggcuGfgAfuuggasgsg | | CCUCCAAUCCAGCCUUCACAUGG | |
| AD-2010007 | ususuuuuAfaUfAfCfgaaccugucuL96 | | asGfsacaGfgUfUfcguaUfuAfaaaaasasa | | UUUUUUUUAAUACGAACCUGUCC | |
| AD-2010032 | asascacuUfuGfAfUfguuaucauuuL96 | | asAfsaugAfuAfAfcaucAfaAfguguusasc | | GUAACACUUUGAUGUUAUCAUUU | |
| AD-2010054 | csasgcuuGfaAfCfUfugagcauacuL96 | | asGfsuauGfcUfCfaaguUfcAfagcugsgsg | | CCCAGCUUGAACUUGAGCAUACA | |
| AD-2010084 | asgscuucUfuUfUfGfauuugcuaauL96 | | asUfsuagCfaAfAfucaaAfaGfaagcusgsa | | UCAGCUUCUUUUGAUUUGCUAAA | |
| AD-2010106 | ascsaucaCfaCfUfUfgcucacauguL96 | | asCfsaugUfgAfGfcaagUfgUfgaugususu | | AAACAUCACACUUGCUCACAUGC | |
| AD-2010123 | asusgccuGfuUfUfAfugcuguucauL96 | | asUfsgaaCfaGfCfauaaAfcAfggcausgsu | | ACAUGCCUGUUUAUGCUGUUCAU | |
| AD-2010142 | asusguugUfuUfAfUfguuucuuacuL96 | | asGfsuaaGfaAfAfcauaAfaCfaacausgsa | | UCAUGUUGUUUAUGUUUCUUACC | |
| AD-2010158 | ususaccuAfgAfAfUfaaauagucuuL96 | | asAfsgacUfaUfUfuauuCfuAfgguaasgsa | | UCUUACCUAGAAUAAAUAGUCUC | |
| AD-2010165 | usascuucUfuUfUfCfccgacuucuuL96 | | asAfsgaaGfuCfGfggaaAfaGfaaguasgsg | | CCUACUUCUUUUCCCGACUUCUU | |
| AD-2010184 | ususacuuUfuUfCfCfuaagauucauL96 | | asUfsgaaUfcUfUfaggaAfaAfaguaasgsa | | UCUUACUUUUUCCUAAGAUUCAG | |
| AD-2010203 | asgsuguaCfaGfCfAfucaugcuccuL96 | | asGfsgagCfaUfGfaugcUfgUfacacusgsa | | UCAGUGUACAGCAUCAUGCUCCA | |
| AD-2010218 | gscsuccaCfaGfCfAfaaccuuccuuL96 | | asAfsggaAfgGfUfuugcUfgUfggagcsasu | | AUGCUCCACAGCAAACCUUCCUA | |
| AD-2010262 | cscsucucAfaAfAfCfcuacauaauuL96 | | asAfsuuaUfgUfAfgguuUfuGfagaggsgsa | | UCCCUCUCAAAACCUACAUAAUA | |
| AD-2010280 | asusuguaUfuUfAfCfcucuccuguuL96 | | asAfscagGfaGfAfgguaAfaUfacaauscsu | | AGAUUGUAUUUACCUCUCCUGUC | |
| AD-2010301 | asasccacAfuUfGfUfuuugaaaauuL96 | | asAfsuuuUfcAfAfaacaAfuGfugguusgsa | | UCAACCACAUUGUUUUGAAAAUA | |
| AD-2010309 | ususucuaUfuUfGfUfgucuccucuuL96 | | asAfsgagGfaGfAfcacaAfaUfagaaasusa | | UAUUUCUAUUUGUGUCUCCUCUA | |
| AD-2010325 | csuscuacUfgCfAfGfuauaaugucuL96 | | asGfsacaUfuAfUfacugCfaGfuagagsgsa | | UCCUCUACUGCAGUAUAAUGUCU | |
| AD-2010361 | gsusguauUfcAfUfCfauugcauucuL96 | | asGfsaauGfcAfAfugauGfaAfuacacsasg | | CUGUGUAUUCAUCAUUGCAUUCC | |
| AD-2010404 | asusggagAfuAfUfCfauugauaaauL96 | | asUfsuuaUfcAfAfugauAfuCfuccaususc | | GAAUGGAGAUAUCAUUGAUAAAU | |
| AD-2010441 | gscsccuuUfuGfAfUfaacagaagcuL96 | | asGfscuuCfuGfUfuaucAfaAfagggcsusu | | AAGCCCUUUUGAUAACAGAAGCC | |
| AD-2010454 | csasgauaGfuCfCfUfuguuuuaauuL96 | | asAfsuuaAfaAfCfaaggAfcUfaucugsgsg | | CCCAGAUAGUCCUUGUUUUAAUG | |
| AD-2010486 | csasccacUfgUfCfUfugaugcucuuL96 | | asAfsgagCfaUfCfaagaCfaGfuggugsasg | | CUCACCACUGUCUUGAUGCUCUG | |
| AD-2010503 | uscsugagCfaAfGfUfuaccucuucuL96 | | asGfsaagAfgGfUfaacuUfgCfucagasgsc | | GCUCUGAGCAAGUUACCUCUUCC | |
| AD-2010521 | uscsccucUfgAfCfCfcucaguuucuL96 | | asGfsaaaCfuGfAfggguCfaGfagggasasg | | CUUCCCUCUGACCCUCAGUUUCC | |
| AD-2010535 | gsusuuccAfuAfUfUfuguaaaauguL96 | | asCfsauuUfuAfCfaaauAfuGfgaaacsusg | | CAGUUUCCAUAUUUGUAAAAUGA | |
| AD-2010545 | asusgagaAfuAfAfAfcauaccaacuL96 | | asGfsuugGfuAfUfguuuAfuUfcucaususu | | AAAUGAGAAUAAACAUACCAACU | |
| AD-2010559 | asascuuaAfuAfAfAfgauauuguguL96 | | asCfsacaAfuAfUfcuuuAfuUfaaguusgsg | | CCAACUUAAUAAAGAUAUUGUGA | |
| AD-2010598 | gsascuagAfaUfGfAfuauuugauauL96 | | asUfsaucAfaAfUfaucaUfuCfuagucsasc | | GUGACUAGAAUGAUAUUUGAUAG | |
| AD-2010609 | asgsaaauUfaAfAfUfgguagcaguuL96 | | asAfscugCfuAfCfcauuUfaAfuuucusasu | | AUAGAAAUUAAAUGGUAGCAGUA | |
| AD-2010624 | gscsaguaUfaAfCfUfauucugaucuL96 | | asGfsaucAfgAfAfuaguUfaUfacugcsusa | | UAGCAGUAUAACUAUUCUGAUCA | |
| AD-2010643 | csascugaCfaUfUfAfauauuccuauL96 | | asUfsaggAfaUfAfuuaaUfgUfcagugsasu | | AUCACUGACAUUAAUAUUCCUAU | |
| AD-2010655 | usgsuuauUfaUfUfCfuuugcucacuL96 | | asGfsugaGfcAfAfagaaUfaAfuaacasasu | | AUUGUUAUUAUUCUUUGCUCACG | |
| AD-2010683 | ascsaacuCfuUfGfUfuuugcuguuuL96 | | asAfsacaGfcAfAfaacaAfgAfguugusasu | | AUACAACUCUUGUUUUGCUGUUG | |
| AD-2010708 | gscsccucUfuUfAfUfguagguuuauL96 | | asUfsaaaCfcUfAfcauaAfaGfagggcsasg | | CUGCCCUCUUUAUGUAGGUUUAC | |
| AD-2010745 | asusacucGfgAfCfUfcaaaugucuuL96 | | asAfsgacAfuUfUfgaguCfcGfaguausasu | | AUAUACUCGGACUCAAAUGUCUC | |
| AD-2010782 | ascsaccaAfaUfGfAfaguggucauuL96 | | asAfsugaCfcAfCfuucaUfuUfgguguscsu | | AGACACCAAAUGAAGUGGUCAUC | |
| AD-2010830 | gsusagcaAfaUfCfAfguauauucuuL96 | | asAfsgaaUfaUfAfcugaUfuUfgcuacsusa | | UAGUAGCAAAUCAGUAUAUUCUA | |
| AD-2010855 | csasaggaAfuAfUfUfaaucuucacuL96 | | asGfsugaAfgAfUfuaauAfuUfccuugsasa | | UUCAAGGAAUAUUAAUCUUCACC | |
| AD-2010871 | uscsacccAfaAfUfUfuugaauuuuuL96 | | asAfsaaaUfuCfAfaaauUfuGfggugasasg | | CUUCACCCAAAUUUUGAAUUUUU | |
| AD-2010911 | gsasgugaUfuUfCfAfuugaauaaauL96 | | asUfsuuaUfuCfAfaugaAfaUfcacucsgsa | | UCGAGUGAUUUCAUUGAAUAAAA | |
| AD-2010919 | asusaaaaUfuCfUfAfcugacuucuuL96 | | asAfsgaaGfuCfAfguagAfaUfuuuaususc | | GAAUAAAAUUCUACUGACUUCUA | |

### Example 17: In Vitro Screening of dsRNA Agents Targeting PLN and CAMK2D

### In Vitro Dual-Luciferase and Endogenous Screening Assays

Hepa1-6 cells cells were transfected by adding 50 μL of siRNA duplexes and 100 ng of a plasmid, comprising partial sequences of human CAMK2D (NM_001321571, e.g., nucleotides 1-2959, or nucleotides 2659-5613), or the sequence of human PLN (NM_002667.5), per well along with 100 μL of Opti-MEM plus 0.5 μL of Lipofectamine 2000 per well (Invitrogen, Carlsbad CA. cat #13778-150) and then incubated at room temperature for 15 minutes. Specifically, V217 plasmid comprises nucleotides 1-2959 of human CAMK2D (NM_001321571), and V218 plasmid comprises nucleotides 2969-5613 of human CAMK2D (NM_001321571). V216 plasmid comprises the sequence of human PLN (NM_002667.5). The mixture was then added to the cells which are re-suspended in 35 μL of fresh complete media. The transfected cells were incubated at 37° C. in an atmosphere of 5% CO2. Single-dose experiments were performed at 10 nM.

Twenty-four hours after the siRNAs and plasmid were transfected, Firefly (transfection control) and Renilla (fused to LEP target sequence) luciferase were measured. First, media was removed from cells. Then Firefly luciferase activity was measured by adding 75 μL of Dual-Glo® Luciferase Reagent equal to the culture medium volume to each well and mix. The mixture was incubated at room temperature for 30 minutes before luminescense (500 nm) was measured on a Spectramax (Molecular Devices) to detect the Firefly luciferase signal. Renilla luciferase activity was measured by adding 75 μL of room temperature of Dual-Glo® Stop & Glo® Reagent to each well and the plates were incubated for 10-15 minutes before luminescence was again measured to determine the Renilla luciferase signal. The Dual-Glo® Stop & Glo® Reagent quenches the firefly luciferase signal and sustained luminescence for the Renilla luciferase reaction. siRNA activity was determined by normalizing the Renilla (LEP) signal to the Firefly (control) signal within each well. The magnitude of siRNA activity was then assessed relative to cells that were transfected with the same vector but were not treated with siRNA or were treated with a non-targeting siRNA. All transfections were done with n=4.

Total RNA Isolation Using DYNABEADS mRNA Isolation Kit (Invitrogen™, Part #: 610-12)

Cells were lysed in 75 μl of Lysis/Binding Buffer containing 3 μL of beads per well and mixed for 10 minutes on an electrostatic shaker. The washing steps were automated on a Biotek EL406, using a magnetic plate support. Beads were washed (in 90 L) once in Buffer A, once in Buffer B, and twice in Buffer E, with aspiration steps in between. Following a final aspiration, complete 10 μL RT mixture was added to each well, as described below.

cDNA Synthesis Using ABI High Capacity cDNA Reverse Transcription Kit (Applied Biosystems, Foster City, CA, Cat #4368813)

A master mix of 1 μl 10× Buffer, 0.4 μL 25× dNTPs, 1 μl Random primers, 0.5 μl Reverse Transcriptase, 0.5 μl RNase inhibitor and 6.6 μl of H₂O per reaction were added per well. Plates were sealed, agitated for 10 minutes on an electrostatic shaker, and then incubated at 37 degrees C. for 2 hours. Following this, the plates were agitated at 80 degrees C. for 8 minutes.

### Real Time PCR

Two microlitre (μl) of cDNA were added to a master mix containing 0.5 μl of human GAPDH TaqMan Probe (4326317E), 0.5 μl human AGT, 2 μl nuclease-free water and 5 μl Lightcycler 480 probe master mix (Roche Cat #04887301001) per well in a 384 well plates (Roche cat #04887301001). Real time PCR was done in a LightCycler480 Real Time PCR system (Roche).

To calculate relative fold change, data were analyzed using the ΔΔCt method and normalized to assays performed with cells transfected with 10 nM AD-1955, or mock transfected cells. IC₅₀s were calculated using a 4 parameter fit model using XLFit and normalized to cells transfected with AD-1955 or mock-transfected. The sense and antisense sequences of AD-1955 are: sense: 5′-cuuAcGcuGAGuAcuucGAdTsdT-3′ and antisense 5′-UCGAAGuACUcAGCGuAAGdTsdT-3′.

The results of the dual-luciferase assays of the agents targeting PLN listed in Tables 39 and 40 are provided in Table 45.

The results of the dual-luciferase assays of the agents targeting CAMK2D listed in Tables 43 and 44 are provided in Table 46.

TABLE 45
Single Dose Reporter Screen for Human PLN in Hepa1-6 Cells

RLuc/FLuc
10 nM

| | % Message | |
|---|---|---|
| Duplex Name | remaining | SD |
| AD-2007034.1 | 74.471 | 1.336 |
| AD-2006965.1 | 79.415 | 2.654 |
| AD-2006935.1 | 98.367 | 7.093 |
| AD-2006926.1 | 73.665 | 2.308 |
| AD-2006862.1 | 71.965 | 3.478 |
| AD-2006839.1 | 72.055 | 3.068 |
| AD-2006764.1 | 80.783 | 2.256 |
| AD-2006751.1 | 94.187 | 1.833 |
| AD-2006743.1 | 78.258 | 2.789 |
| AD-2006731.1 | 75.200 | 2.414 |
| AD-2006658.1 | 63.797 | 3.636 |
| AD-2006640.1 | 79.573 | 3.340 |
| AD-2006626.1 | 89.767 | 1.977 |
| AD-2006548.1 | 79.254 | 2.656 |
| AD-2006525.1 | 97.983 | 1.930 |
| AD-2006451.1 | 71.323 | 1.207 |
| AD-2006437.1 | 78.733 | 3.978 |
| AD-2006362.1 | 68.489 | 1.876 |
| AD-2006347.1 | 57.744 | 1.454 |
| AD-2006334.1 | 90.091 | 3.499 |
| AD-2006259.1 | 80.739 | 2.113 |
| AD-2006242.1 | 69.201 | 4.156 |
| AD-2006234.1 | 69.430 | 2.572 |
| AD-2006164.1 | 85.985 | 2.877 |
| AD-2006150.1 | 78.944 | 2.370 |
| AD-2006132.1 | 65.844 | 3.148 |
| AD-2006119.1 | 67.891 | 2.715 |
| AD-2006056.1 | 79.256 | 3.796 |
| AD-2006046.1 | 72.080 | 2.814 |
| AD-2006035.1 | 74.951 | 1.204 |
| AD-2006018.1 | 69.416 | 1.116 |
| AD-2005954.1 | 71.976 | 1.319 |
| AD-2005942.1 | 73.672 | 3.299 |
| AD-2005923.1 | 84.576 | 1.308 |
| AD-2005862.1 | 52.157 | 0.938 |
| AD-2005849.1 | 88.045 | 2.389 |
| AD-2005826.1 | 112.005 | 0.917 |
| AD-2005760.1 | 111.182 | 2.680 |
| AD-2005737.1 | 81.084 | 2.698 |
| AD-2005720.1 | 67.458 | 0.088 |
| AD-2005704.1 | 66.982 | 2.264 |
| AD-2005687.1 | 90.730 | 1.279 |
| AD-2005678.1 | 73.201 | 2.059 |
| AD-2005658.1 | 81.316 | 2.647 |
| AD-2005638.1 | 76.024 | 1.690 |
| AD-2005617.1 | 69.415 | 0.770 |
| AD-2005604.1 | 62.339 | 1.020 |
| AD-2005588.1 | 73.296 | 3.091 |
| AD-2005575.1 | 73.973 | 2.813 |
| AD-2005557.1 | 86.620 | 2.652 |
| AD-2005530.1 | 87.155 | 2.522 |
| AD-2005527.1 | 66.437 | 3.464 |
| AD-2005485.1 | 60.734 | 1.125 |
| AD-2005471.1 | 74.209 | 2.816 |
| AD-2005447.1 | 55.255 | 1.527 |
| AD-2005429.1 | 61.881 | 2.504 |
| AD-2005409.1 | 55.910 | 1.679 |
| AD-2005396.1 | 70.051 | 1.457 |
| AD-2005384.1 | 59.529 | 1.880 |
| AD-2005365.1 | 49.761 | 2.254 |
| AD-2005350.1 | 49.862 | 1.778 |
| AD-2005325.1 | 49.062 | 1.550 |
| AD-2005300.1 | 72.649 | 3.503 |
| AD-2005287.1 | 66.711 | 1.750 |
| AD-2005274.1 | 57.620 | 2.185 |
| AD-2005250.1 | 42.331 | 0.718 |
| AD-2005234.1 | 36.497 | 1.216 |
| AD-2005221.1 | 39.891 | 0.782 |
| AD-2005193.1 | 47.444 | 1.638 |
| AD-2005179.1 | 31.061 | 0.664 |
| AD-2005164.1 | 38.766 | 1.143 |
| AD-2005146.1 | 42.489 | 1.460 |
| AD-2005129.1 | 35.148 | 1.370 |
| AD-2005108.1 | 45.362 | 1.516 |
| AD-2005095.1 | 44.466 | 0.712 |
| AD-2005082.1 | 44.276 | 0.629 |
| AD-2005067.1 | 35.862 | 0.419 |
| AD-2005051.1 | 36.352 | 0.285 |
| AD-2005033.1 | 35.934 | 1.159 |
| AD-2005020.1 | 52.416 | 1.682 |
| AD-2005002.1 | 43.338 | 1.433 |
| AD-2004989.1 | 33.202 | 0.616 |
| AD-2004970.1 | 39.309 | 0.534 |
| AD-2004950.1 | 36.412 | 1.258 |
| AD-2004935.1 | 39.050 | 0.834 |
| AD-2004922.1 | 55.716 | 2.384 |
| AD-2004909.1 | 37.126 | 0.682 |
| AD-2004896.1 | 31.389 | 0.294 |
| AD-2004885.1 | 43.271 | 1.438 |
| AD-2004872.1 | 36.214 | 2.665 |
| AD-2004853.1 | 39.615 | 1.714 |
| AD-2004837.1 | 32.899 | 1.054 |
| AD-2004825.1 | 29.761 | 1.078 |
| AD-2004806.1 | 32.790 | 1.262 |
| AD-2004790.1 | 29.823 | 1.134 |
| AD-2004777.1 | 33.226 | 1.762 |
| AD-2004763.1 | 30.181 | 1.410 |
| AD-2004747.1 | 28.097 | 0.601 |
| AD-2004718.1 | 47.433 | 3.268 |
| AD-2004689.1 | 29.233 | 0.737 |
| AD-2004673.1 | 35.458 | 0.769 |
| AD-2004660.1 | 29.217 | 0.864 |
| AD-2004646.1 | 42.910 | 1.006 |
| AD-2004632.1 | 23.375 | 0.587 |
| AD-2004608.1 | 84.486 | 2.159 |
| AD-2004594.1 | 10.840 | 0.844 |
| AD-2004575.1 | 6.793 | 0.323 |
| AD-2004555.1 | 6.065 | 0.193 |
| AD-2004539.1 | 4.753 | 0.229 |
| AD-2004518.1 | 4.717 | 0.315 |
| AD-2004495.1 | 4.114 | 0.253 |
| AD-2004481.1 | 64.414 | 0.700 |
| AD-2004468.1 | 5.628 | 0.493 |
| AD-2004458.1 | 7.073 | 0.385 |
| AD-2004428.1 | 8.520 | 0.514 |
| AD-2004400.1 | 25.806 | 0.707 |
| AD-2004386.1 | 3.955 | 0.105 |
| AD-2004373.1 | 7.681 | 0.321 |
| AD-2004360.1 | 5.221 | 0.397 |
| AD-2004345.1 | 9.997 | 0.707 |
| AD-2004329.1 | 5.616 | 0.469 |
| AD-2004319.1 | 4.875 | 0.267 |
| AD-2004301.1 | 10.902 | 0.526 |
| AD-2004284.1 | 6.918 | 0.616 |
| AD-2004260.1 | 13.058 | 0.327 |
| AD-2004238.1 | 7.854 | 0.197 |
| AD-2004202.1 | 7.385 | 0.540 |
| AD-2004189.1 | 10.326 | 0.405 |
| AD-2004176.1 | 12.764 | 0.435 |
| AD-2004156.1 | 11.300 | 1.182 |
| AD-2004142.1 | 12.730 | 0.159 |
| AD-2004113.1 | 8.975 | 0.279 |
| AD-2004095.1 | 2.922 | 0.151 |
| AD-2004080.1 | 59.209 | 1.282 |
| AD-2004072.1 | 13.064 | 0.967 |
| Postive control | 0.538 | 0.095 |

TABLE 46
Single Dose Reporter Screen for
Human CAMK2D in Hepa1-6 Cells

| | RLuc/Fluc V218 | | RLuc/Fluc V217 | |
|---|---|---|---|---|
| | 10 nM | | 10 nM | |
| | % Message | | % Message | |
| Duplex Name | Remaining | SD | Remaining | SD |
| AD-2010919.1 | 54.775 | 3.875 | | |
| AD-2010911.1 | 63.205 | 0.834 | | |
| AD-2010871.1 | 87.416 | 5.684 | | |
| AD-2010855.1 | 93.304 | 3.325 | | |
| AD-2010830.1 | 84.938 | 3.881 | | |
| AD-2010782.1 | 78.337 | 4.600 | | |
| AD-2010745.1 | 88.461 | 6.353 | | |
| AD-2010708.1 | 93.117 | 6.451 | | |
| AD-2010683.1 | 69.483 | 3.261 | | |
| AD-2010655.1 | 77.639 | 2.283 | | |
| AD-2010643.1 | 87.707 | 3.018 | | |
| AD-2010624.1 | 80.114 | 3.333 | | |
| AD-2010609.1 | 90.918 | 4.832 | | |
| AD-2010598.1 | 74.414 | 1.039 | | |
| AD-2010559.1 | 86.988 | 1.703 | | |
| AD-2010545.1 | 71.871 | 3.138 | | |
| AD-2010535.1 | 73.669 | 2.725 | | |
| AD-2010521.1 | 71.072 | 0.767 | | |
| AD-2010503.1 | 72.548 | 1.061 | | |
| AD-2010486.1 | 72.378 | 2.071 | | |
| AD-2010454.1 | 84.009 | 4.651 | | |
| AD-2010441.1 | 74.567 | 2.349 | | |
| AD-2010404.1 | 79.424 | 3.393 | | |
| AD-2010361.1 | 76.688 | 2.472 | | |
| AD-2010325.1 | 79.238 | 4.044 | | |
| AD-2010309.1 | 67.564 | 2.345 | | |
| AD-2010301.1 | 59.701 | 2.046 | | |
| AD-2010280.1 | 72.425 | 2.744 | | |
| AD-2010262.1 | 81.376 | 4.975 | | |
| AD-2010218.1 | 67.650 | 4.358 | | |
| AD-2010203.1 | 67.587 | 2.183 | | |
| AD-2010184.1 | 68.670 | 2.029 | | |
| AD-2010165.1 | 76.258 | 1.121 | | |
| AD-2010158.1 | 93.459 | 0.881 | | |
| AD-2010142.1 | 79.214 | 1.961 | | |
| AD-2010123.1 | 68.582 | 1.827 | | |
| AD-2010106.1 | 68.511 | 1.973 | | |
| AD-2010084.1 | 65.765 | 2.122 | | |
| AD-2010054.1 | 55.070 | 1.970 | | |
| AD-2010032.1 | 61.999 | 2.320 | | |
| AD-2010007.1 | 80.181 | 2.149 | | |
| AD-2009987.1 | 86.662 | 5.464 | | |
| AD-2009959.1 | 52.258 | 1.295 | | |
| AD-2009931.1 | 76.826 | 2.364 | | |
| AD-2009901.1 | 79.035 | 4.350 | | |
| AD-2009881.1 | 89.987 | 6.509 | | |
| AD-2009852.1 | 85.627 | 5.840 | | |
| AD-2009839.1 | 81.043 | 3.015 | | |
| AD-2009823.1 | 92.060 | 3.075 | | |
| AD-2009755.1 | 97.673 | 4.670 | | |
| AD-2009722.1 | 7.458 | 0.308 | | |
| AD-2009707.1 | 8.020 | 0.492 | | |
| AD-2009690.1 | 15.120 | 0.831 | | |
| AD-2009655.1 | 6.171 | 0.266 | | |
| AD-2009643.1 | 13.322 | 0.233 | | |
| AD-2009615.1 | 6.271 | 0.252 | | |
| AD-2009599.1 | 26.536 | 0.816 | | |
| AD-2009584.1 | 8.085 | 0.842 | | |
| AD-2009568.1 | 6.779 | 0.301 | | |
| AD-2009546.1 | 12.216 | 0.260 | | |
| AD-2009537.1 | 7.405 | 0.140 | | |
| AD-2009525.1 | 8.702 | 0.322 | | |
| AD-2009505.1 | 15.844 | 0.558 | | |
| AD-2009490.1 | 7.311 | 0.324 | | |
| AD-2009437.1 | 10.483 | 0.217 | | |
| AD-2009425.1 | 3.552 | 0.159 | | |
| AD-2009386.1 | 7.732 | 0.356 | | |
| AD-2009369.1 | 4.180 | 0.202 | | |
| AD-2009354.1 | 12.511 | 0.483 | | |
| AD-2009344.1 | 19.310 | 0.115 | | |
| AD-2009317.1 | 7.006 | 0.297 | | |
| AD-2009302.1 | 3.416 | 0.110 | | |
| AD-2009285.1 | 8.156 | 0.520 | | |
| AD-2009253.1 | 4.172 | 0.270 | | |
| AD-2009238.1 | 3.640 | 0.161 | | |
| AD-2009211.1 | 12.916 | 0.346 | | |
| AD-2009197.1 | 2.605 | 0.169 | | |
| AD-2009162.1 | 9.455 | 0.439 | | |
| AD-2009147.1 | 8.493 | 0.118 | | |
| AD-2009122.1 | 3.398 | 0.118 | | |
| AD-2009108.1 | 5.204 | 0.207 | | |
| AD-2009078.1 | 2.664 | 0.213 | | |
| AD-2009053.1 | 6.560 | 0.119 | | |
| AD-2009021.1 | 9.111 | 0.335 | | |
| AD-2008977.1 | 30.950 | 1.078 | | |
| AD-2008941.1 | 24.568 | 0.808 | | |
| AD-2008921.1 | 37.944 | 1.475 | | |
| AD-2008899.1 | 4.925 | 0.148 | | |
| AD-2008875.1 | 28.692 | 0.747 | | |
| AD-2008860.1 | 7.795 | 0.347 | | |
| AD-2008844.1 | 7.289 | 0.395 | | |
| AD-2008835.1 | 3.943 | 0.103 | | |
| AD-2008799.1 | 4.375 | 0.238 | | |
| AD-2008767.1 | 5.444 | 0.182 | | |
| AD-2008746.1 | 2.286 | 0.105 | | |
| AD-2008720.1 | 12.460 | 1.301 | | |
| AD-2008714.1 | 28.171 | 0.547 | 45.65886104 | 1.1035907 |
| AD-2008708.1 | 33.037 | 0.902 | 61.79586966 | 1.5307934 |
| AD-2008701.1 | 16.450 | 0.554 | 44.17280687 | 1.9294042 |
| AD-2008687.1 | 2.892 | 0.059 | 16.20778676 | 0.3737978 |
| AD-2008666.1 | 1.191 | 0.085 | 11.77972984 | 0.3063456 |
| AD-2008639.1 | 1.494 | 0.085 | 16.08336566 | 0.8901049 |
| AD-2008623.1 | 2.640 | 0.112 | 17.62525171 | 0.4686597 |
| AD-2008590.1 | 1.714 | 0.101 | 14.38598257 | 0.6394634 |
| AD-2008575.1 | 3.631 | 0.077 | 19.99313108 | 1.1687672 |
| AD-2008555.1 | 11.072 | 1.039 | 31.50514332 | 1.917178 |
| AD-2008540.1 | 2.454 | 0.076 | 20.53179586 | 0.2485112 |
| AD-2008516.1 | 1.405 | 0.050 | 21.48519022 | 0.3653849 |
| AD-2008474.1 | | | 24.02537918 | 0.6280613 |
| AD-2008453.1 | | | 13.73531117 | 0.3217469 |
| AD-2008402.1 | | | 16.87679869 | 0.6515435 |
| AD-2008375.1 | | | 16.98560683 | 0.498232 |
| AD-2008357.1 | | | 14.21209621 | 0.3004723 |
| AD-2008325.1 | | | 16.73471701 | 0.7296915 |
| AD-2008310.1 | | | 19.32436409 | 0.8544198 |
| AD-2008288.1 | | | 14.23682319 | 0.3745798 |
| AD-2008273.1 | | | 71.50461253 | 3.2380485 |
| AD-2008257.1 | | | 27.49900574 | 1.3455372 |
| AD-2008242.1 | | | 10.76358 | 0.5053741 |
| AD-2008227.1 | | | 15.28452231 | 0.4654998 |
| AD-2008211.1 | | | 13.60926615 | 0.1644877 |
| AD-2008170.1 | | | 25.66489061 | 0.7888178 |
| AD-2008116.1 | | | 51.30787029 | 3.2940949 |
| AD-2008093.1 | | | 27.98984317 | 0.9882612 |
| AD-2008065.1 | | | 21.13859066 | 0.4752274 |
| AD-2008047.1 | | | 48.37926908 | 0.6189311 |
| AD-2008019.1 | | | 43.49619485 | 1.3081996 |
| AD-2007985.1 | | | 19.09148666 | 0.2746135 |
| AD-2007974.1 | | | 51.99112086 | 2.5656624 |
| AD-2007934.1 | | | 33.97294662 | 1.0110332 |
| AD-2007870.1 | | | 36.17811695 | 2.5952914 |
| AD-2007856.1 | | | 32.58840548 | 0.8877862 |
| AD-2007841.1 | | | 26.72922 | 1.5211521 |
| AD-2007818.1 | | | 24.81585399 | 0.7996233 |
| AD-2007801.1 | | | 10.70960323 | 0.5182464 |
| AD-2007785.1 | | | 12.3854364 | 0.1588502 |
| AD-2007759.1 | | | 17.54481777 | 0.8691434 |
| AD-2007743.1 | | | 18.65843633 | 0.8691339 |
| AD-2007705.1 | | | 20.97710268 | 2.0623631 |
| AD-2007702.1 | | | 22.23837144 | 1.2792613 |
| AD-2007687.1 | | | 14.28983278 | 0.5877183 |
| AD-2007668.1 | | | 12.55123358 | 0.5519322 |
| AD-2007653.1 | | | 16.03158735 | 1.0494897 |
| AD-2007627.1 | | | 8.778782361 | 0.5807514 |
| AD-2007604.1 | | | 11.3277006 | 0.5801774 |
| AD-2007585.1 | | | 12.12409743 | 0.3406555 |
| AD-2007568.1 | | | 10.91668988 | 0.9796381 |
| AD-2007549.1 | | | 28.5985825 | 1.8602411 |
| AD-2007528.1 | | | 26.24339901 | 3.0900046 |
| AD-2007508.1 | | | 24.20127046 | 2.0456574 |
| AD-2007490.1 | | | 32.98630016 | 1.863752 |
| AD-2007468.1 | | | 52.37705677 | 2.902928 |
| AD-2007451.1 | | | 14.12308396 | 0.8174496 |
| AD-2007420.1 | | | 12.9988447 | 1.0455565 |
| AD-2007404.1 | | | 73.82535862 | 3.2250002 |
| AD-2007389.1 | | | 23.12555471 | 0.7699604 |
| AD-2007325.1 | | | 40.76464069 | 2.3558281 |
| AD-2007269.1 | | | 13.45774901 | 0.9639825 |
| AD-2007238.1 | | | 26.19603597 | 0.5648448 |
| AD-2007211.1 | | | 48.54309211 | 2.5350914 |
| AD-2007160.1 | | | 10.07170671 | 0.3209378 |
| AD-2007144.1 | | | 12.95174443 | 0.6263839 |
| AD-2007079.1 | | | 11.53212195 | 0.5785039 |
| AD-2007012.1 | | | 19.95795834 | 0.7006737 |
| AD-2006886.1 | | | 31.6192243 | 0.2459024 |
| AD-2006870.1 | | | 27.09699643 | 1.5404928 |
| AD-2006710.1 | | | 19.12964642 | 1.8183614 |
| AD-2006610.1 | | | 10.23483771 | 0.9179456 |
| AD-2006588.1 | | | 14.25744683 | 0.8293527 |
| AD-2006501.1 | | | 36.6319817 | 3.3363464 |
| AD-2006473.1 | | | 20.91053622 | 1.3427681 |
| AD-2006395.1 | | | 15.13240856 | 1.3434645 |
| AD-2006307.1 | | | 11.78307666 | 0.4056504 |
| AD-2006275.1 | | | 18.57181389 | 1.7504964 |
| AD-2006091.1 | | | 58.62441069 | 4.1443315 |
| AD-2005989.1 | | | 33.17064508 | 1.9442207 |
| AD-2005914.1 | | | 27.87696239 | 0.6755636 |
| AD-2005899.1 | | | 36.94068638 | 0.6020162 |
| AD-2005876.1 | | | 40.88704238 | 2.1567636 |
| | | | 37.2476383 | 0.6411107 |
| Positive control | 0.455 | 0.108 | 0.781 | 0.124 |

1. A double stranded ribonucleic acid (dsRNA) agent for inhibiting expression of a target gene, comprising
an antisense strand which is complementary to the target gene;
a sense strand which is complementary to the antisense strand and forms a double stranded region with the antisense strand; and
one or more C₂₂ hydrocarbon chains conjugated to one or more internal positions on at least one strand.

2. The dsRNA agent of claim 1, wherein the C₂₂ hydrocarbon chain is saturated or unsaturated.

3. The dsRNA agent of claim 1, wherein the C₂₂ hydrocarbon chain is linear or branched

4.-8. (canceled)

9. The dsRNA agent of claim 1, wherein the one or more C₂₂ hydrocarbon chains are conjugated to one or more of the following internal positions: positions 4-8 and 13-18 on the sense strand, and positions 6-10 and 15-18 on the antisense strand, counting from the 5′ end of each strand.

10. (canceled)

11. The dsRNA agent of claim 9, wherein the one or more C₂₂ hydrocarbon chains are conjugated to position 6 on the sense strand, counting from the 5′-end of the sense strand.

12. The dsRNA agent of claim 1, wherein the sense the antisense strands are each independently 15-30 nucleotides in length; 19 to 25 nucleotides in length; or 21 to 23 nucleotides in length.

13. The dsRNA agent of claim 1, wherein the one or more C₂₂ hydrocarbon chains is an aliphatic, alicyclic, or polyalicyclic compound.

14. (canceled)

15. The dsRNA agent of claim 1, wherein the one or more C₂₂ hydrocarbon chains is a C₂₂ acid, a C₂₂ alcohol, or a C₂₂ amide.

16.-20. (canceled)

21. The dsRNA agent of claim 1, wherein the one or more C₂₂ hydrocarbon chains is conjugated via a carrier that replaces one or more nucleotide(s) in the internal position(s).

22. (canceled)

23. The dsRNA agent of claim 1, wherein the one or more C₂₂ hydrocarbon chains is conjugated to the dsRNA agent via a linker containing an ether, thioether, urea, carbonate, amine, amide, maleimide-thioether, disulfide, phosphodiester, sulfonamide linkage, a product of a click reaction, or carbamate.

24. The dsRNA agent of claim 1, wherein the one or more C₂₂ hydrocarbon chains is conjugated to the dsRNA agent via a linker or a carrier or via internucleotide phosphate linkage.

25. The dsRNA agent of claim 1, wherein the dsRNA agent comprises at least one single-stranded overhang.

26. (canceled)

27. The dsRNA agent of claim 1, wherein at least one end of the dsRNA agent is blunt-ended.

28. The dsRNA agent of claim 1, wherein the one or more C22 hydrocarbon chains is conjugated to a nucleobase, sugar moiety, or internucleosidic phosphate linkage.

29. (canceled)

30. (canceled)

31. The dsRNA agent of claim 1, further comprising a modified phosphate at the 5′-end of the antisense strand.

32. The dsRNA agent of claim 1, wherein the one or more C₂₂ hydrocarbon chains or targeting ligand is conjugated via a biocleavable linker selected from the group consisting of a DNA linker, an RNA linker, a disulfide linker, an amide linker, a protease cleavable peptide linker, a functionalized monosaccharide linker, a galactosamine oligosaccharide linker, a glucosamine linker, a glucose linker, a galactose linker, a mannose linker, and combinations thereof.

33. The dsRNA agent of claim 1, wherein the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

34. The dsRNA agent of claim 1, wherein the target gene is selected from the group consisting of adrenoceptor beta 1 (ADRB1); calcium voltage-gated channel subunit alpha1 C (CACNA1C); calcium voltage-gated channel subunit alpha1 G (CACNA1G) (T type calcium channel); angiotensin II receptor type 1(AGTR1); Sodium Voltage-Gated Channel Alpha Subunit 2 (SCN2A); Hyperpolarization Activated Cyclic Nucleotide Gated Potassium Channel 1 (HCN1); Hyperpolarization Activated Cyclic Nucleotide Gated Potassium Channel 4 (HCN4); Hyperpolarization Activated Cyclic Nucleotide Gated Potassium Channel 3 (HCN3); Potassium Voltage-Gated Channel Subfamily A Member 5 (KCNA5); Potassium Inwardly Rectifying Channel Subfamily J Member 3 (KCNJ3); Potassium Inwardly Rectifying Channel Subfamily J Member 4 (KCNJ4); phospholamban (PLN); calcium/calmodulin dependent protein kinase II delta (CAMK2D); Phosphodiesterase 1 (PDE1), Delta 4-Desaturase, Sphingolipid 1 (DEGS1): leptin (LEP); folliculin (FLCN): Zinc Finger Protein 423 (ZFP423); Cyclin Dependent Kinase 6 (CDK6); Regulatory Associated Protein Of MTOR Complex 1 (RPTOR); Mechanistic Target Of Rapamycin Kinase, (mTOR): Forkhead Box P1 (FOXP1); Phosphodiesterase 3B (PDE3B); Activin A Receptor Type 1C (ACVR1C), myostatin (MSTN); Cholinergic Receptor Nicotinic Alpha 1 Subunit (CHRNA1): Cholinergic Receptor Nicotinic Beta 1 Subunit (CHRNB11): Cholinergic Receptor Nicotinic Delta Subunit (CHRND): Cholinergic Receptor Nicotinic Epsilon Subunit (CHRNE); Cholinergic Receptor Nicotinic Gamma Subunit (CHRNG): Collagen Type XIII Alpha 1 Chain (COL13A1): Docking Protein 7 (DOK7): LDL Receptor Related Protein 4 (LRP4): Muscle Associated Receptor Tyrosine Kinase (MUSK): Receptor Associated Protein Of The Synapse (RAPSN): Sodium Voltage-Gated Channel Alpha Subunit 4 (SCN4A); and Double Homeobox 4 (DUX4).

35. (canceled)

36. (canceled)

37. A cell containing the dsRNA agent of claim 1.

38. A pharmaceutical composition for inhibiting expression of the target gene, comprising the dsRNA agent of claim 1.

39. A method of inhibiting expression of a target gene in a skeletal muscle cell, a cardiac muscle cell, or an adipocyte, comprising contacting the cell with a dsRNA agent that inhibits expression of a target gene, wherein the dsRNA agent comprises
an antisense strand which is complementary to the target gene;
a sense strand which is complementary to the antisense strand and forms a double stranded region with the antisense strand; and
one or more C₂₂ hydrocarbon chains conjugated to one or more internal positions on at least one strand.

40.-75. (canceled)

76. A method of treating a subject having a skeletal muscle disorder, a cardiac muscle disorder, or an adipose tissue disorder, comprising:
administering to the subject a therapeutically effective amount of the dsRNA agent of claim 1, thereby treating the subject.

77.-84. (canceled)

85. The method of claim 76, further comprising administering to the subject an additional agent or a therapy suitable for treatment or prevention of a cardiac muscle disorder, or an adipose tissue disorder.

86. (canceled)

87. (canceled)

88. A method of synthesizing a nucleoside monomer having the structure of Formula (I): 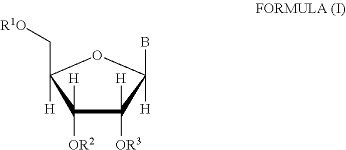
wherein:
B is a modified or unmodified nucleobase;
R¹ is a hydroxyl protecting group;
R² is H or phosphoramidite;
R³ is C₂₂H₄₅
wherein the compound of formula (I) is free or substantially free of a compound of Formula (II) 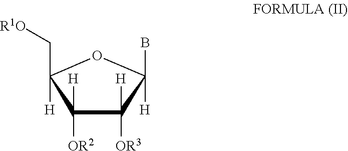
wherein:
B is a modified or unmodified nucleobase;
R¹ is a hydroxyl protecting group;
R² is C₂₂H₄₅
R³ is H

89. (canceled)
